(19) United States
(12) Patent Application Publication
(10) Pub. No.: US 2013/0012485 A1
(43) Pub. Date: Jan. 10, 2013

(54) ORGANIC COMPOUNDS

(76) Inventors: Daniel Kaspar Bäschlin, Arlesheim (CH); Richard Sedrani, Basel (CH); Stefanie Flohr, Reinach (CH); Kenji Namoto, Basel (CH); Nils Ostermann, Binzen (DE); Finton Sirockin, Blotzheim (FR); François Gessier, Altkirch (FR); Garry Fenton, Harlow (GB); Mandy Christine Beswick, Harlow (GB); David Edward Clark, Harlow (GB); Bohdan Waszkowycz, Harlow (GB)

(21) Appl. No.: 12/520,323

(22) PCT Filed: Dec. 20, 2007
(86) PCT No.: PCT/EP2007/011304

§ 371 Date: Jul. 10, 2009

(30) Foreign Application Priority Data
Dec. 22, 2006 (EP) 06127001.3

(51) Int. Cl. A61K 31/135; A61K 31/568; A61K 31/565; C07C 217/52; C07D 487/04; A61K 31/4985; C07D 211/08; A61K 31/451; C07D 295/135; A61K 31/495; C07D 401/04; A61K 31/4545; A61K 31/454; C07D 243/08; A61K 31/551; C07D 223/04; A61K 31/55; A61K 31/501; C07D 237/14; A61K 31/50; A61P 3/10; A61P 19/02; A61P 3/04; A61P 19/10; A61P 9/04; A61P 25/28; A61P 13/12; A61P 3/06; A61P 9/10; A61P 29/00; A61P 25/00; A61P 35/04; A61P 9/12; A61P 25/22; A61P 5/00; C07C 215/42

(52) U.S. Cl. 514/171; 564/442; 514/646; 564/443; 544/350; 514/249; 546/229; 514/331; 544/402; 514/255.03; 546/188; 514/316; 546/208; 514/326; 540/492; 514/218; 540/484; 514/212.01; 544/238; 514/252.03; 544/239; 514/247

(57) ABSTRACT

Compounds of the formula (I) are provided:

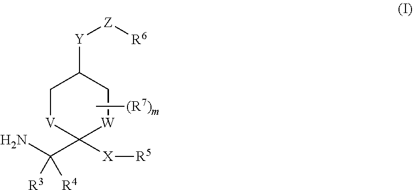

wherein V, W, X, Y, Z, R³, R⁴, R⁵, R⁶, R⁷ and m are as defined in the specification; and pharmaceutically acceptable salts and prodrugs thereof. The compounds may be useful in the treatment or prevention of various diseases and conditions in which dipeptidylpeptidase-IV (DPP-IV) is implicated.

### FIELD OF THE INVENTION

The present invention relates to compounds and their use in therapy.

### BACKGROUND TO THE INVENTION

Dipeptidylpeptidase-IV (DPP-IV) is a serine protease which cleaves N-terminal dipeptides from a peptide chain containing, in general, a praline residue in the penultimate position. DPP-IV is widely expressed in mammalian tissue as a type II integral membrane protein. The protease is expressed on the surface of differentiated epithelial cells of the intestine, liver, kidney proximal tubules, prostate, corpus luteum, and on leukocyte subsets such as lymphocytes and macrophages. A soluble form of the enzyme is found in serum that has structure and function identical to the membrane-bound form of the enzyme but lacks the hydrophobic transmembrane domain.

DPP-IV has many physiologically relevant substrates including chemokines (e.g. eotaxin and macrophage-derived chemokine), neuropeptides (e.g. neuropeptide Y and substance P), vasoactive peptides, and incretins (e.g. GLP-1 and GIP). GLP-1 (glucagon-like peptide-1) is a hormone produced in the L cells of the distal small intestine in response to ingested nutrients. GLP-1 receptor binding on various tissues stimulates insulin gene expression, biosynthesis and glucose-dependent insulin secretion, inhibits glucagon secretion, promotes satiety, slows gastric emptying and promotes growth of pancreatic beta cells.

Although the biological role of DPP-IV in mammalian systems has not been completely established, it is believed to play an important role in neuropeptide metabolism, T-cell activation, attachment of cancer cells to the endothelium and the entry of HIV into lymphoid cells. It has also been discovered that DPP-IV is responsible for inactivating glucagon-like peptide-1 (GLP-1). Since GLP-1 is a major stimulator of pancreatic insulin secretion and has direct beneficial effects on glucose disposal, DPP-IV inhibition appears to represent an attractive approach for treating, for example, non-insulin-dependent diabetes mellitus (NIDDM).

DPP-IV has also been shown to play a part in the immune response. Expressed by T-CD4+ lymphocytes, where it is synonymous with the antigen CD26, DPP-IV plays an important part in the mechanism of transplant rejection (Transplantation 1997, 63 (10), 1495-500). By allowing more selective suppression of the immune response, inhibition of DPP-IV accordingly represents an extremely promising approach in the prevention of transplant rejection in transplant patients.

Inhibitors of DPP-IV are described inter alia in WO-A-03/000180, WO-A-000181, WO-A-004498, WO-A-03/082817, WO-A-04/032836, WO-A-04/007468 and WO-A-05/121089.

WO 03/063797 discloses the following compounds as intermediates for the synthesis of inhibitors of potassium ion channel function:

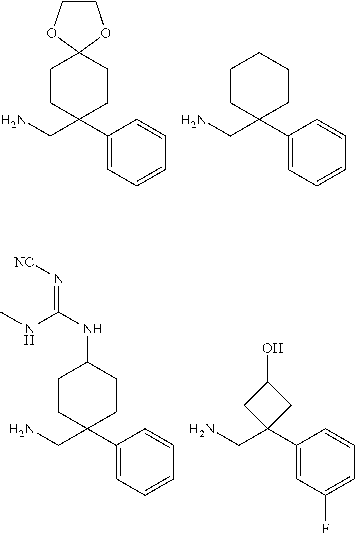

In addition, WO 2005/105096 discloses the following compounds as intermediates for the synthesis of inhibitors of potassium ion channel function:

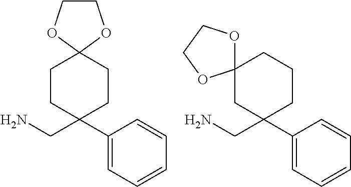

WO 03/000676 describes the following compound as being useful in the treatment of malaria:

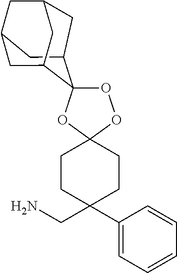 [SMILES: NCC1(c2ccccc2)CCC2(CC1)OOC1(O2)C2CC3CC(C2)CC1C3]

### SUMMARY OF THE INVENTION

According to the invention there is provided a compound of the Formula (I):

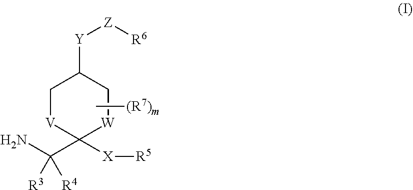

wherein
one of V and W is selected from a bond, —(CH₂)ₙ—, —O—, —NH— and —N(R⁸)—; and the other is selected from a bond, —(CH₂)ₙ— and —O—;
X is a bond or a linker having 1 to 5 in-chain atoms and comprising one or more linkages selected from —O—, —C(O)—, —S(O)ₗ—, —N(R⁸)— and hydrocarbylene optionally substituted with 1, 2, 3, 4 or 5 R¹⁰; with the proviso that, when at least one of V and W is —O—, —NH— or —N(R⁸)—, X is a bond;
Y is a bond; or Y and an R⁷ moiety taken together with the atom(s) to which they are attached form a carbocycle or a heterocycle, either of which is optionally substituted with 1, 2, 3, 4 or 5 R¹⁰, and may be saturated or unsaturated;
Z is a bond or a linker having 1 to 12 in-chain atoms and comprising one or more linkages selected from —O—, —C(O)—, —S(O)ₗ—, —N(R⁸)—, hydrocarbylene optionally substituted with 1, 2, 3, 4 or 5 R¹⁰, and heterocyclylene optionally substituted with 1, 2, 3, 4 or 5 R¹⁰;
R³ and R⁴ are each independently hydrogen or R¹⁰; or R³ and R⁴ taken together with the carbon atom to which they are attached form carbocyclyl or heterocyclyl, either of which is optionally substituted with 1, 2, 3, 4 or 5 R¹⁰;
R⁵ is selected from hydrogen, except when X is a bond; hydrocarbyl optionally substituted with 1, 2, 3, 4 or 5 R¹⁰; and —(CH₂)ₖ-heterocyclyl optionally substituted with 1, 2, 3, 4 or 5 R¹⁰;
R⁶ is selected from hydrogen, except when Y and Z are each a bond; hydrocarbyl optionally substituted with 1, 2, 3, 4 or 5 R¹⁰; and —(CH₂)ₖ-heterocyclyl optionally substituted with 1, 2, 3, 4 or 5 R¹⁰;
R⁷ is independently selected from R¹⁰;
or two R⁷ moieties taken together may form a bridge between the atoms to which they are attached, wherein the bridge is a hydrocarbylene or —(CH₂)ᵢ—O—(CH₂)ⱼ— bridge, wherein i and j are each independently 0, 1 or 2;
R⁸ is selected from R⁹, —OR⁹, —C(O)R⁹, —C(O)OR⁹ and —S(O)ₗR⁹;
R⁹ is selected from hydrogen; hydrocarbyl optionally substituted with 1, 2, 3, 4 or 5 R¹⁰; and —(CH₂)ₖ-heterocyclyl optionally substituted with 1, 2, 3, 4 or 5 R¹⁰;
each R¹⁰ is independently selected from halogen, trifluoromethyl, cyano, nitro, oxo, ═NR¹¹, —OR¹¹, —C(O)R¹¹, —C(O)OR¹¹, —OC(O)R¹¹, —S(O)ₗR¹¹, —N(R¹¹)R¹², —C(O)N(R¹¹)R¹², —S(O)ₗN(R¹¹)R¹² and R¹³;
R¹¹ and R¹² are each independently hydrogen or R¹³;
R¹³ is selected from hydrocarbyl and —(CH₂)ₖ-heterocyclyl, either of which is optionally substituted with 1, 2, 3, 4 or 5 substituents independently selected from halogen, cyano, amino, hydroxy, C₁₋₆ alkyl and C₁₋₆ alkoxy;
k is 0, 1, 2, 3, 4, 5 or 6;
l is 0, 1 or 2;
m is 0, 1, 2, 3, 4, 5 or 6; and
n is 1 or 2;
   or a pharmaceutically acceptable salt or prodrug thereof.

Also provided are pharmaceutical formulations comprising a compound of the invention and, optionally, a pharmaceutically acceptable diluent or carrier.

The invention also provides a product comprising a compound of the invention and a therapeutic agent; as a combined preparation for simultaneous, separate or sequential use in therapy.

Compounds of the invention may be useful in the treatment or prevention of a disease or condition selected from non-insulin-dependent diabetes mellitus, arthritis, obesity, allograft transplantation, calcitonin-osteoporosis, heart failure, impaired glucose metabolism or impaired glucose tolerance, neurodegenerative diseases, cardiovascular or renal diseases, and neurodegenerative or cognitive disorders. Compounds of the invention may also be useful for producing a sedative or anxiolytic effect, attenuating post-surgical catabolic changes or hormonal responses to stress, reducing mortality and morbidity after myocardial infarction, modulating hyperlipidemia or associated conditions, or lowering VLDL, LDL or Lp(a) levels. Accordingly, other aspects of the invention concern the use of the present compounds in such therapies and the use of the compounds for the manufacture of a medicament for use in such therapies. Therapeutic methods comprising administering a therapeutically effective amount of a compound of the invention to a patient are also provided.

The compounds of the invention can exist in different forms, such as free acids, free bases, esters and other prodrugs, salts and tautomers, for example, and the disclosure includes all variant forms of the compounds.

The extent of protection includes counterfeit or fraudulent products which contain or purport to contain a compound of the invention irrespective of whether they do in fact contain such a compound and irrespective of whether any such compound is contained in a therapeutically effective amount.

Included in the scope of protection are packages which include a description or instructions which indicate that the package contains a species or pharmaceutical formulation of the invention and a product which is or comprises, or purports to be or comprise, such a formulation or species. Such packages may be, but are not necessarily, counterfeit or fraudulent.

Features, integers, characteristics, compounds, chemical moieties or groups described in conjunction with a particular aspect, embodiment or example of the invention are to be understood to be applicable to any other aspect, embodiment or example described herein unless incompatible therewith.

### DESCRIPTION OF VARIOUS EMBODIMENTS

### Hydrocarbyl and Hydrocarbylene

The terms “hydrocarbyl” and “hydrocarbylene” as used herein include reference to moieties consisting exclusively of hydrogen and carbon atoms; such a moiety may comprise an aliphatic and/or an aromatic moiety. The moiety may comprise 1, 2, 3, 4, 5, 6, 7, 8, 9, 10, 11, 12, 13, 14, 15, 16, 17, 18, 19 or 20 carbon atoms. Examples of hydrocarbyl groups include C₁₋₆ alkyl (e.g. C₁, C₂, C₃ or C₄ alkyl, for example methyl, ethyl, propyl, isopropyl, n-butyl, sec-butyl or tert-butyl); C₁₋₆ alkyl substituted by aryl (e.g. benzyl) or by cycloalkyl (e.g cyclopropylmethyl); cycloalkyl (e.g. cyclopropyl, cyclobutyl, cyclopentyl or cyclohexyl); alkenyl (e.g. 2-butenyl); alkynyl (e.g. 2-butynyl); aryl (e.g. phenyl, naphthyl or fluorenyl) and the like.

### Alkyl

The terms “alkyl” and “C₁₋₆ alkyl” as used herein include reference to a straight or branched chain alkyl moiety having 1, 2, 3, 4, 5 or 6 carbon atoms. This term includes reference to groups such as methyl, ethyl, propyl (n-propyl or isopropyl), butyl (n-butyl, sec-butyl or tert-butyl), pentyl, hexyl and the like. In particular, alkyl may have 1, 2, 3 or 4 carbon atoms.

### Alkenyl

The terms “alkenyl” and “C₂₋₆ alkenyl” as used herein include reference to a straight or branched chain alkyl moiety having 2, 3, 4, 5 or 6 carbon atoms and having, in addition, at least one double bond, of either E or Z stereochemistry where applicable. This term includes reference to groups such as ethenyl, 2-propenyl, 1-butenyl, 2-butenyl, 3-butenyl, 1-pentenyl, 2-pentenyl, 3-pentenyl, 1-hexenyl, 2-hexenyl and 3-hexenyl and the like.

### Alkynyl

The terms “alkynyl” and “C₂₋₆ alkynyl” as used herein include reference to a straight or branched chain alkyl moiety having 2, 3, 4, 5 or 6 carbon atoms and having, in addition, at least one triple bond. This term includes reference to groups such as ethynyl, 1-propynyl, 2-propynyl, 1-butynyl, 2-butynyl, 3-butynyl, 1-pentynyl, 2-pentynyl, 3-pentynyl, 1-hexynyl, 2-hexynyl and 3-hexynyl and the like.

### Alkoxy

The terms “alkoxy” and “C₁₋₆ alkoxy” as used herein include reference to —O-alkyl, wherein alkyl is straight or branched chain and comprises 1, 2, 3, 4, 5 or 6 carbon atoms. In one class of embodiments, alkoxy has 1, 2, 3 or 4 carbon atoms. This term includes reference to groups such as methoxy, ethoxy, propoxy, isopropoxy, butoxy, tert-butoxy, pentoxy, hexoxy and the like.

### Cycloalkyl

The term “cycloalkyl” as used herein includes reference to an alicyclic moiety having 3, 4, 5, 6, 7 or 8 carbon atoms. The group may be a bridged or polycyclic ring system. More often cycloalkyl groups are monocyclic. This term includes reference to groups such as cyclopropyl, cyclobutyl, cyclopentyl, cyclohexyl, norbornyl, bicyclo[2.2.2]octyl and the like.

### Aryl

The term “aryl” as used herein includes reference to an aromatic ring system comprising 6, 7, 8, 9, 10, 11, 12, 13, 14, 15 or 16 ring carbon atoms. Aryl is often phenyl but may be a polycyclic ring system, having two or more rings, at least one of which is aromatic. This term includes reference to groups such as phenyl, naphthyl, fluorenyl, azulenyl, indenyl, anthryl and the like.

### Carbocyclyl

The term “carbocyclyl” as used herein includes reference to a saturated (e.g. cycloalkyl) or unsaturated (e.g. aryl) ring moiety having 3, 4, 5, 6, 7, 8, 9, 10, 11, 12, 13, 14, 15 or 16 carbon ring atoms. In particular, carbocyclyl includes a 3- to 10-membered ring or ring system and, in particular, a 5- or 6-membered ring, which may be saturated or unsaturated. A carbocyclic moiety is, for example, selected from cyclopropyl, cyclobutyl, cyclopentyl, cyclohexyl, norbornyl, bicyclo[2.2.2]octyl, phenyl, naphthyl, fluorenyl, azulenyl, indenyl, anthryl and the like.

### Heterocyclyl

The term “heterocyclyl” as used herein includes reference to a saturated (e.g. heterocycloalkyl) or unsaturated (e.g. heteroaryl) heterocyclic ring moiety having from 3, 4, 5, 6, 7, 8, 9, 10, 11, 12, 13, 14, 15 or 16 ring atoms, at least one of which is selected from nitrogen, oxygen, phosphorus, silicon and sulphur. In particular, heterocyclyl includes a 3- to 10-membered ring or ring system and more particularly a 5- or 6-membered ring, which may be saturated or unsaturated.

A heterocyclic moiety is, for example, selected from oxiranyl, azirinyl, 1,2-oxathiolanyl, imidazolyl, thienyl, furyl, tetrahydrofuryl, pyranyl, thiopyranyl, thianthrenyl, isobenzofuranyl, benzofuranyl, chromenyl, 2H-pyrrolyl, pyrrolyl, pyrrolinyl, pyrrolidinyl, imidazolyl, imidazolidinyl, benzimidazolyl, pyrazolyl, pyrazinyl, pyrazolidinyl, thiazolyl, isothiazolyl, dithiazolyl, oxazolyl, isoxazolyl, pyridyl, pyrazinyl, pyrimidinyl, piperidyl, piperazinyl, pyridazinyl, morpholinyl, thiomorpholinyl, especially thiomorpholino, indolizinyl, isoindolyl, 3H-indolyl, indolyl, benzimidazolyl, cumaryl, indazolyl, triazolyl, tetrazolyl, purinyl, 4H-quinolizinyl, isoquinolyl, quinolyl, tetrahydroquinolyl, tetrahydroisoquinolyl, decahydroquinolyl, octahydroisoquinolyl, benzofuranyl, dibenzofuranyl, benzothiophenyl, dibenzothiophenyl, phthalazinyl, naphthyridinyl, quinoxalyl, quinazolinyl, quinazolinyl, cinnolinyl, pteridinyl, carbazolyl, p-carbolinyl, phenanthridinyl, acridinyl, perimidinyl, phenanthrolinyl, furazanyl, phenazinyl, phenothiazinyl, phenoxazinyl, chromenyl, isochromanyl, chromanyl and the like.

### Heterocycloalkyl

The term “heterocycloalkyl” as used herein includes reference to a saturated heterocyclic moiety having 3, 4, 5, 6 or 7 ring carbon atoms and 1, 2, 3, 4 or 5 ring heteroatoms selected from nitrogen, oxygen, phosphorus and sulphur. The group may be a polycyclic ring system but more often is monocyclic. This term includes reference to groups such as azetidinyl, pyrrolidinyl, tetrahydrofuranyl, piperidinyl, oxiranyl, pyrazolidinyl, imidazolyl, indolizidinyl, piperazinyl, thiazolidinyl, morpholinyl, thiomorpholinyl, quinolizidinyl and the like.

### Heteroaryl

The term “heteroaryl” as used herein includes reference to an aromatic heterocyclic ring system having 5, 6, 7, 8, 9, 10, 11, 12, 13, 14, 15 or 16 ring atoms, at least one of which is selected from nitrogen, oxygen and sulphur. The group may be a polycyclic ring system, having two or more rings, at least one of which is aromatic, but is more often monocyclic. This term includes reference to groups such as pyrimidinyl, furanyl, benzo[b]thiophenyl, thiophenyl, pyrrolyl, imidazolyl, pyrrolidinyl, pyridinyl, benzo[b]furanyl, pyrazinyl, purinyl, indolyl, benzimidazolyl, quinolinyl, phenothiazinyl, triazinyl, phthalazinyl, 2H-chromenyl, oxazolyl, isoxazolyl, thiazolyl, isoindolyl, indazdyl, purinyl, isoquinolinyl, quinazolinyl, pteridinyl and the like.

### Halogen

The term “halogen” as used herein includes reference to F, Cl, Br or I. In a particular, halogen may be F or Cl, of which F is more common.

### Substituted

The term “substituted” as used herein in reference to a moiety means that one or more, especially up to 5, more especially 1, 2 or 3, of the hydrogen atoms in said moiety are replaced independently of each other by the corresponding number of the described substituents. The term “optionally substituted” as used herein means substituted or unsubstituted.

It will, of course, be understood that substituents are only at positions where they are chemically possible, the person skilled in the art being able to decide (either experimentally or theoretically) without inappropriate effort whether a particular substitution is possible. For example, amino or hydroxy groups with free hydrogen may be unstable if bound to carbon atoms with unsaturated (e.g. olefinic) bonds. Additionally, it will of course be understood that the substituents described herein may themselves be substituted by any substituent, subject to the aforementioned restriction to appropriate substitutions as recognised by the skilled man.

### Pharmaceutically Acceptable

The term “pharmaceutically acceptable” as used herein includes reference to those compounds, materials, compositions, and/or dosage forms which are, within the scope of sound medical judgment, suitable for use in contact with the tissues of human beings or animals without excessive toxicity, irritation, allergic response, or other problem or complication, commensurate with a reasonable benefit/risk ratio. This term includes acceptability for both human and veterinary purposes.

### Independently

Where two or more moieties are described as being “each independently” selected from a list of atoms or groups, this means that the moieties may be the same or different. The identity of each moiety is therefore independent of the identities of the one or more other moieties.

### Compounds

The invention provides compounds of the Formula (I):

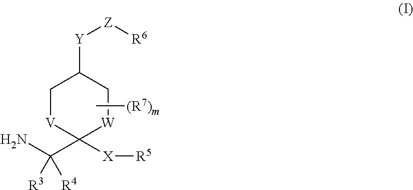

wherein V, W, X, Y, Z, R¹, R², R³, R⁴, R⁵, R⁶, R⁷ and m are as defined herein;
or a pharmaceutically acceptable salt or prodrug thereof.

In embodiments, the compound is not one of the following compounds:

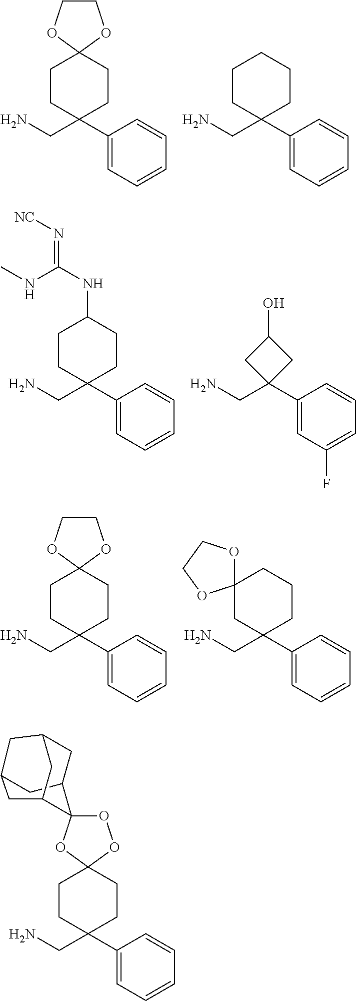

Further embodiments of the invention are described below. It will be appreciated that the features specified in each embodiment may be combined with other specified features, to provide further embodiments.

### V & W

In Formula (I), one of V and W is selected from a bond, —(CH₂)ₙ—, —O—, —NH— and —N(R⁸)—; and the other is selected from a bond, —(CH₂)ₙ— and —O—; wherein n is 1 or 2. Usually, n is 1. It will be appreciated that any —NH— or —CH₂— group present may be unsubstituted or substituted with one or more R⁷. Also, as mentioned above, when at least one of V and W is —O—, —NH— or —N(R⁸)—, X is a bond.

The invention includes compounds in which the ring shown in Formula (I) is a 5-membered ring, e.g. compounds of the following Formulae:

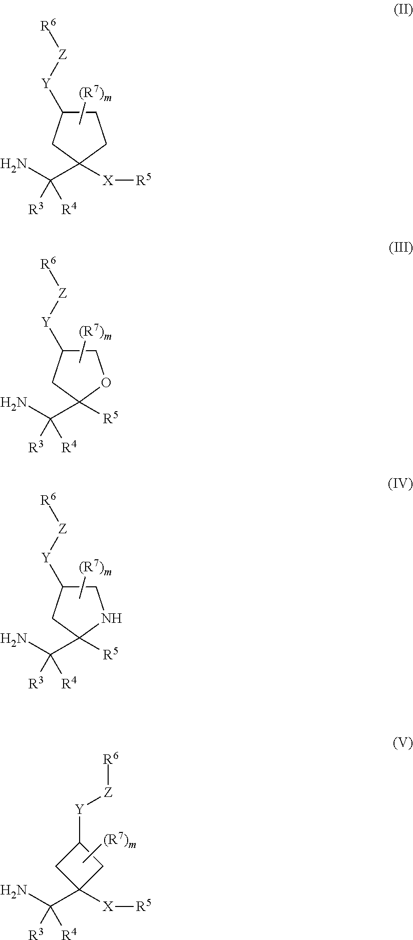

or, in each case, a pharmaceutically acceptable salt or prodrug thereof.

Of particular mention are compounds of the formula (II)) and pharmaceutically acceptable salts or prodrugs thereof.

The invention also includes compounds in which the ring shown in Formula (I) is a 6-membered ring, e.g. compounds of the following Formulae:

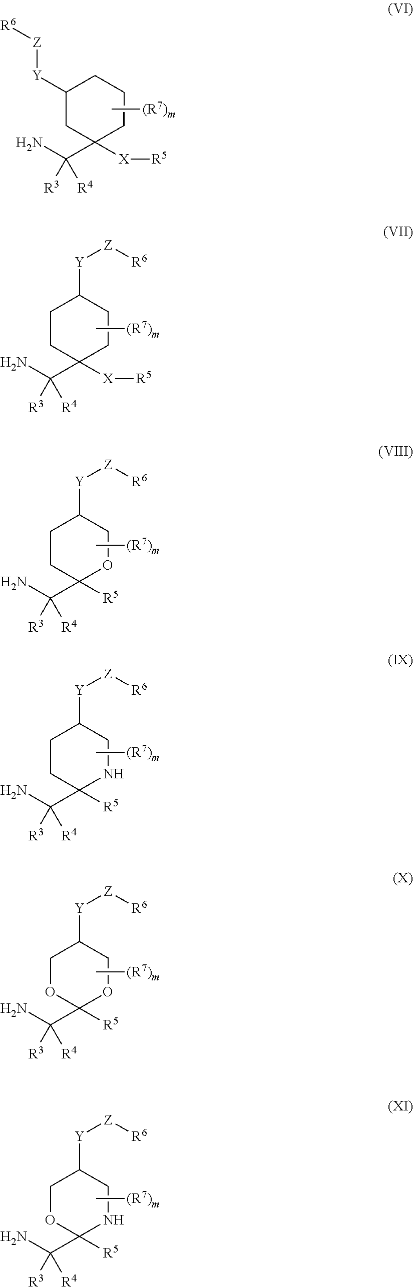

or, in each case, a pharmaceutically acceptable salt or prodrug thereof.

The invention also includes compounds in which the ring shown in Formula (I) is a 7- or 8-membered ring, e.g. compounds of the following Formulae:

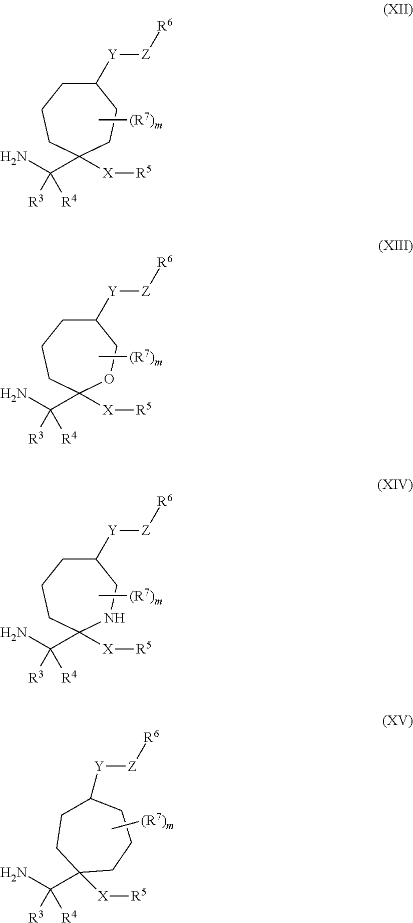

or, in each case, a pharmaceutically acceptable salt or prodrug thereof.

Of particular mention are compounds of the Formula (VII) and pharmaceutically acceptable salts or prodrugs thereof.

In other embodiments, —NH— ring moieties shown in the above Formulae are replaced by —N(R⁸)—, wherein R⁸ is other than hydrogen.

### R³ & R⁴

R³ and R⁴ are each independently hydrogen or R¹⁰; or R³ and R⁴ taken together with the carbon atom to which they are attached form carbocyclyl or heterocyclyl, either of which is optionally substituted with 1, 2, 3, 4 or 5 R¹⁰.

In one embodiment, R³ and R⁴ are each independently hydrogen; C₁, C₂, C₃ or C₄ alkyl, for example methyl, ethyl, propyl, isopropyl, n-butyl, sec-butyl or tert-butyl, any of which is optionally substituted with 1, 2, 3 or 4 halogen (e.g. fluorine or chlorine) atoms, an example being trifluoromethyl; or C₁, C₂, C₃ or C₄ alkoxy, for example methoxy, ethoxy, propoxy, isopropoxy, butoxy, tert-butoxy, any of which is optionally substituted with 1, 2, 3 or 4 halogen (e.g. fluorine or chlorine) atoms.

In another embodiment, R³ is hydrogen; C₁, C₂, C₃ or C₄ alkyl, for example methyl, ethyl, propyl, isopropyl, n-butyl, sec-butyl or tert-butyl, any of which is optionally substituted with 1, 2, 3 or 4 halogen (e.g. fluorine or chlorine) atoms, an example being trifluoromethyl; or C₁, C₂, C₃ or C₄ alkoxy, for example methoxy, ethoxy, propoxy, isopropoxy, butoxy or tert-butoxy, any of which is optionally substituted with 1, 2, 3 or 4 halogen (e.g. fluorine or chlorine) atoms; and R⁴ is typically hydrogen.

In a further embodiment, R³ is hydrogen or C₁₋₆ alkyl; and R⁴ is hydrogen.

In a further embodiment, R³ is hydrogen or methyl; and R⁴ is hydrogen.

In a further embodiment, R³ and R⁴ taken together with the carbon atom to which they are attached form cycloalkyl or heterocycloalkyl, either of which is optionally substituted with 1, 2, 3, 4 or 5 R¹⁰. Examples of cycloalkyl groups include cyclopropyl, cyclobutyl, cyclopentyl or cyclohexyl, any of which is optionally substituted with 1, 2, 3, 4 or 5 R¹⁰. Examples of heterocycloalkyl groups include azetidinyl, pyrrolidinyl, piperidinyl, piperazinyl or morpholinyl, any of which is optionally substituted with 1, 2, 3, 4 or 5 R¹⁰. The or each R¹⁰ may be, for example, hydroxy, halogen (for example, chlorine or fluorine); C₁, C₂, C₃ or C₄ alkyl, for example methyl, ethyl, propyl, isopropyl, n-butyl, sec-butyl or tert-butyl, any of which is optionally substituted with 1, 2, 3 or 4 halogen (e.g. fluorine or chlorine) atoms, an example being trifluoromethyl; or C₁, C₂, C₃ or C₄ alkoxy, for example methoxy, ethoxy, propoxy, isopropoxy, butoxy or tert-butoxy, any of which is optionally substituted with 1, 2, 3 or 4 halogen (e.g. fluorine or chlorine) atoms.

In a further embodiment, R³ and R⁴ are each hydrogen. The invention therefore includes compounds of the following Formula:

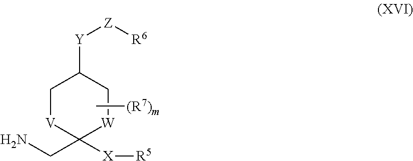

or a pharmaceutically acceptable salt or prodrug thereof.

### —X—R⁵

X is a bond or a linker having 1 to 5 in-chain atoms and comprising one or more linkages selected from —O—, —C(O)—, —S(O)ₗ)—, —N(R⁸)— and hydrocarbylene optionally substituted with 1, 2, 3, 4 or 5 R¹⁰; wherein R⁸ is selected from R⁹, —OR⁹, —C(O)R⁹, —C(O)OR⁹ and —S(O)ₗR⁹; and wherein R⁹ is selected from hydrogen; hydrocarbyl optionally substituted with 1, 2, 3, 4 or 5 R¹⁰; and —(CH₂)ₖ-heterocyclyl optionally substituted with 1, 2, 3, 4 or 5 R¹⁰. R⁸ is often hydrogen or C₁₋₆ alkyl optionally substituted with 1, 2, 3, 4 or 5 R¹⁰. Also, when at least one of V and W is —O—, —NH— or —N(R⁸)—, X is a bond.

In one embodiment, X is selected from the following linkers:
—X¹—;
—X¹—X²—;
—X¹—X²—X³—;
—X¹—X²—X³—X⁴—; and
—X¹—X²—X³—X⁴—X⁵—;
   wherein X₁, X², X³, X⁴ and X⁵ are each independently selected from —O—, —C(O)—, —S(O)ₗ—, —N(R⁸)— and hydrocarbylene (e.g. C₁₋₅ alkylene) optionally substituted with 1, 2, 3, 4 or 5 R¹⁰. More usually, X is —X¹— or —X¹—X²—.

In another embodiment, X is a bond or a linker comprising 1, 2 or 3 linkages selected from selected from —O—, —C(O)—, —S(O)ₗ—, —N(R⁸)— and —CH₂—. The linker typically comprises 1, 2 or 3 in-chain atoms. Thus, X may be selected from a bond, —O—, —C(O)—, —S(O)ₗ—, —N(R⁸)—, —CH₂—, —CH₂CH₂—, —OCH₂—, —OCH₂CH₂—, —CH₂O—, —CH₂CH₂O— and —CH₂OCH₂—. In certain compounds, X is selected from a bond, —CH₂— and —O—.

R⁵ is selected from hydrogen, except when X is a bond; hydrocarbyl optionally substituted with 1, 2, 3, 4 or 5 R¹⁰; and —(CH₂)ₖ-heterocyclyl optionally substituted with 1, 2, 3, 4 or 5 R¹⁰.

In one embodiment, R⁵ is hydrogen and X is other than a bond.

In another embodiment, R⁵ is hydrocarbyl optionally substituted with 1, 2, 3, 4 or 5 R¹⁰. In this case, R⁵ is often selected from C₁₋₆ alkyl (e.g. C₁, C₂, C₃ or C₄ alkyl) or —(CH₂)ₖ— carbocyclyl (e.g. —(CH₂)ₖ-cycloalkyl or —(CH₂)ₖ-aryl), either of which is optionally substituted with 1, 2, 3, 4 or 5 R¹⁰. In particular, R⁵ may be C₁₋₆ alkyl (e.g. C₁, C₂, C₃ or C₄ alkyl), —(CH₂)ₖ-cycloalkyl (e.g. cyclopropyl or cyclopropylmethyl) or —(CH₂)ₖ-aryl (e.g. phenyl or benzyl), any of which is optionally substituted with 1, 2, 3, 4 or 5 R₁₀.

In a further embodiment, R⁵ is —(CH₂)ₖ-heterocyclyl optionally substituted with 1, 2, 3, 4 or 5 R¹⁰. Typically, k is 0 or 1, more usually 0. The heterocyclyl group may be heterocycloalkyl or heteroaryl, either of which is optionally substituted with 1, 2, 3, 4 or 5 R¹⁰. The heterocyclyl group may be monocyclic or bicyclic, usually monocyclic. Exemplary heterocyclyl groups include oxiranyl, azirinyl, 1,2-oxathiolanyl, imidazolyl, thienyl, furyl, tetrahydrofuryl, pyranyl, thiopyranyl, thianthrenyl, isobenzofuranyl, benzofuranyl, chromenyl, 2H-pyrrolyl, pyrrolyl, pyrrolinyl, pyrrolidinyl, imidazolyl, imidazolidinyl, benzimidazolyl, pyrazolyl, pyrazinyl, pyrazolidinyl, thiazolyl, isothiazolyl, dithiazolyl, oxazolyl, isoxazolyl, pyridyl, pyrazinyl, pyrimidinyl, piperidyl, piperazinyl, pyridazinyl, morpholinyl, thiomorpholinyl, especially thiomorpholino, indolizinyl, isoindolyl, 3H-indolyl, indolyl, benzimidazolyl, cumaryl, indazolyl, triazolyl, tetrazolyl, purinyl, 4H-quinolizinyl, isoquinolyl, quinolyl, tetrahydroquinolyl, tetrahydroisoquinolyl, decahydroquinolyl, octahydroisoquinolyl, benzofuranyl, dibenzofuranyl, benzothiophenyl, dibenzothiophenyl, phthalazinyl, naphthyridinyl, quinoxalyl, quinazolinyl, quinazolinyl, cinnolinyl, pteridinyl, carbazolyl, p-carbolinyl, phenanthridinyl, acridinyl, perimidinyl, phenanthrolinyl, furazanyl, phenazinyl, phenothiazinyl, phenoxazinyl, chromenyl, isochromanyl and chromanyl, any of which is optionally substituted with 1, 2, 3, 4 or 5 R¹⁰.

In a further embodiment, R⁵ is carbocyclyl or heterocyclyl, either of which is optionally substituted with 1, 2, 3, 4 or 5 R¹⁰.

In a further embodiment, R⁵ is aryl or heteroaryl, either of which is optionally substituted with 1, 2, 3, 4 or 5 R¹⁰.

In a further embodiment, R⁵ is aryl, in particular phenyl or naphthyl, either of which is optionally substituted with 1, 2, 3, 4 or 5 R¹⁰. In embodiments, R⁵ is phenyl optionally substituted with 1, 2, 3, 4 or 5 R¹⁰, wherein the or each R¹⁰ is, for example, hydroxy, halogen (for example, chlorine or fluorine); C₁, C₂, C₃ or C₄ alkyl, for example methyl, ethyl, propyl, isopropyl, n-butyl, sec-butyl or tert-butyl, any of which is optionally substituted with 1, 2, 3 or 4 halogen (e.g. fluorine or chlorine) atoms, an example being trifluoromethyl; or C₁, C₂, C₃ or C₄ alkoxy, for example methoxy, ethoxy, propoxy, isopropoxy, butoxy, tert-butoxy, any of which is optionally substituted with 1, 2, 3 or 4 halogen (e.g. fluorine or chlorine) atoms. For example, R⁵ may be phenyl optionally substituted with 1, 2, 3, 4 or 5 halogen (e.g. fluorine or chlorine) atoms.

In a further embodiment, R⁵ is heteroaryl (often monocyclic), for example, thienyl or benzothiophenyl, and is optionally substituted with 1, 2, 3, 4 or 5 R¹⁰, wherein the or each R¹⁰ is, for example, hydroxy, halogen (for example, chlorine or fluorine); C₁, C₂, C₃ or C₄ alkyl, for example methyl, ethyl, propyl, isopropyl, n-butyl, sec-butyl or tert-butyl, any of which is optionally substituted with 1, 2, 3 or 4 halogen (e.g. fluorine or chlorine) atoms, an example being trifluoromethyl; or C₁, C₂, C₃ or C₄ alkoxy, for example methoxy, ethoxy, propoxy, isopropoxy, butoxy, tert-butoxy, any of which is optionally substituted with 1, 2, 3 or 4 halogen (e.g. fluorine or chlorine) atoms.

In further embodiment, X is a bond or a linker comprising 1, 2 or 3 linkages selected from selected from —O—, —C(O)—, —S(O)ₗ—, —N(R⁸)— and —CH₂—; and R⁵ is selected from C₁₋₆ alkyl, cycloalkyl, aryl (e.g. phenyl) and heterocyclyl (e.g. pyridinyl or pyrrolidinone, in particular pyrrolidin-2-one), any of which is optionally substituted with 1, 2, 3, 4 or 5 R¹⁰. In particular, X may be selected from a bond, —CH₂— and —O—.

The invention includes a compound of the following Formula:

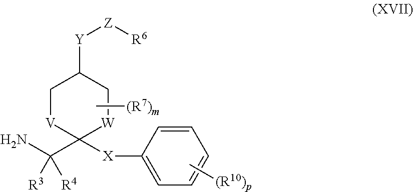

wherein p is 0, 1, 2, 3, 4 or 5;
or a pharmaceutically acceptable salt or prodrug thereof.

With regard to Formula (XVIII), X is often a bond or a linker comprising 1, 2 or 3 linkages selected from —O—, —C(O)—, —S(O)ₗ—, —N(R⁸)— and —CH₂—. For example, X may be selected from a bond, —CH₂— and —O—.

In particular, the invention includes compounds of the following Formula:

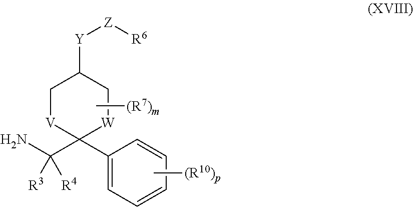

or a pharmaceutically acceptable salt or prodrug thereof.

Also of mention are compounds of the following Formula:

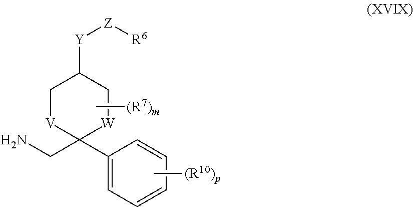

or a pharmaceutically acceptable salt or prodrug thereof.

In embodiments of the above formulae, when p is 1, 2, 3, 4 or 5, at least one R¹⁰ is halogen or C₁₋₆ alkyl. In particular embodiments, the or each R¹⁰ is independently halogen or C₁₋₆ alkyl.

In other embodiments, when p is 1, 2, 3, 4 or 5, at least one R¹⁰ is halogen. In particular embodiments, the or each R¹⁰ is halogen.

In further embodiments, when p is 1, 2, 3, 4 or 5, at least one R¹⁰ is fluorine or chlorine. In particular embodiments, the or each R¹⁰ is independently fluorine or chlorine. Of particular mention are compounds in which —X—R⁵ is 2-chlorophenyl.

In further embodiments, p is 0, 1, 2 or 3. In particular embodiments, p is 0, 1 or 2.

### Y

Y is a bond; or Y and an R⁷ moiety taken together with the atom(s) to which they are attached form a carbocycle or a heterocycle, either of which is optionally substituted with 1, 2, 3, 4 or 5 R¹⁰, and may be saturated or unsaturated.

In one embodiment, Y is a bond. The invention therefore includes compounds of the following Formula:

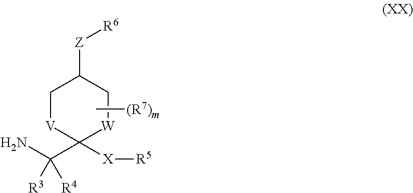

or a pharmaceutically acceptable salt or prodrug thereof.

In another embodiment, Y and an R⁷ moiety are attached to adjacent ring carbon atoms and taken together with those atoms form a carbocycle or a heterocycle, either of which is optionally substituted with 1, 2, 3, 4 or 5 R¹⁰.

The invention therefore includes compounds of the following Formula:

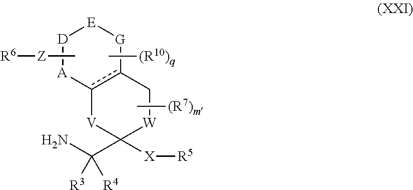

wherein
A, D and G are each independently selected from —C(O)—, —(CH₂)ₙ—, ═CH—, —NH—, ═N—, —O—, and —S(O)ₗ—;
E is selected from a bond, —C(O)—, —(CH₂)ₙ—, ═CH—, —NH—, ═N—, —O—, and —S(O)ₗ—;
m′ is 0, 1, 2, 3, 4 or 5;
q is 0, 1, 2, 3, 4 or 5; and
---- represents an optional second bond;
or a pharmaceutically acceptable salt or prodrug thereof.

It will be appreciated that any —CH₂—, ═CH— or —NH— group present may be unsubstituted or substituted with one or more substituents selected from —Z—R⁶ (when other than hydrogen) and R¹⁰ moieties.

In certain compounds, A is selected from —C(O)—, —O—, —S— and —CH₂—; D and G are each independently selected from —CH₂—, ═CH—, —NH— and ═N—; and E is selected from a bond, —CH₂— and CH.

The invention includes compounds of the following Formulae:

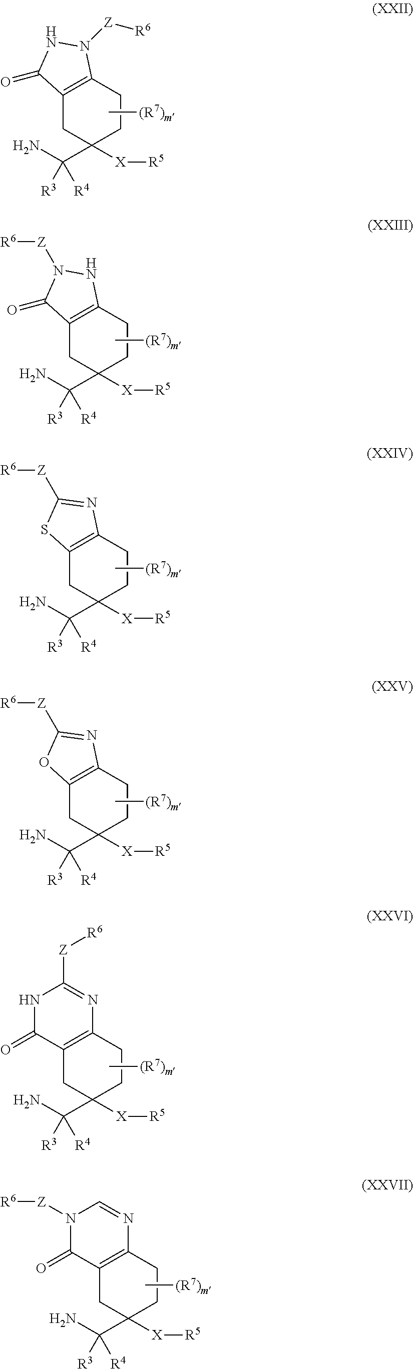

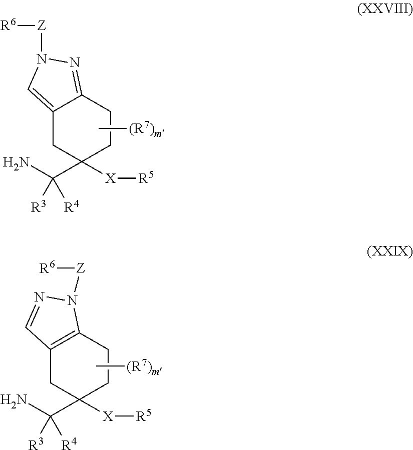

or, in each case, a pharmaceutically acceptable salt or prodrug thereof.

In another embodiment, Y and an R⁷ moiety are attached to the same carbon atom and taken together with that atom form a carbocycle or a heterocycle, either of which is optionally substituted with 1, 2, 3, 4 or 5 R¹⁰, and may be saturated or unsaturated.

The invention therefore includes compounds of the following Formula:

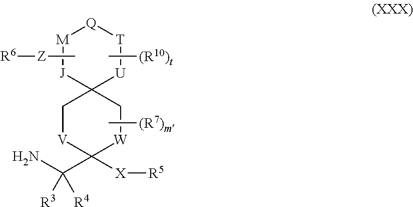

wherein
J, M, T and U are each independently selected from —C(O)—, —(CH₂)ₙ—, —NH—, —O— and —S(O)ₗ—;
Q is selected from a bond, —C(O)—, —(CH₂)ₙ—, —O—, —NH— and —S(O)ₗ—;
m′ is 0, 1, 2, 3, 4 or 5; and
t is 0, 1, 2, 3, 4 or 5;
or a pharmaceutically acceptable salt or prodrug thereof.

It will be appreciated that any —CH₂— or —NH— group present may be unsubstituted or substituted with one or more substituents selected from —Z—R⁶ (when other than hydrogen) and R¹⁰ moieties.

In certain compounds, J, M, T and U are each independently selected from —CH₂— and —NH—; and Q is selected from a bond, —CH₂— and —NH—.

The invention also includes compounds of the following Formulae:

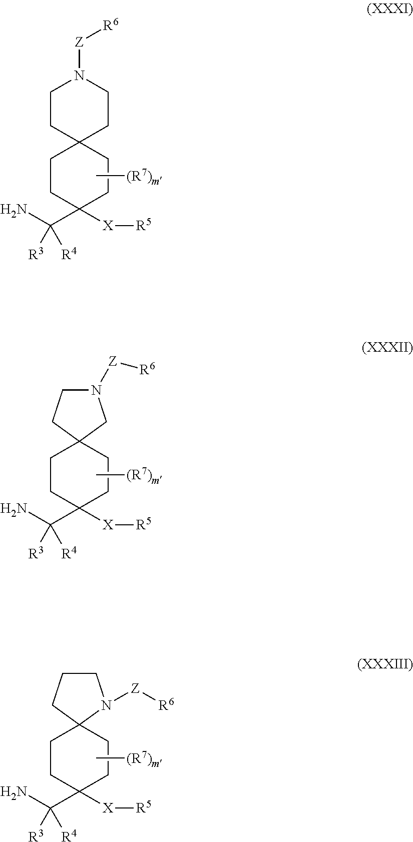

or, in each case, a pharmaceutically acceptable salt or prodrug thereof.

### —Z—R⁶

Z is a bond or a linker having 1 to 12 in-chain atoms and comprising one or more linkages selected from —O—, —C(O)—, —S(O)ₗ—, —N(R⁸)—, hydrocarbylene optionally substituted with 1, 2, 3, 4 or 5 R¹⁰, and heterocyclylene optionally substituted with 1, 2, 3, 4 or 5 R¹⁰; wherein R⁸ is selected from R⁹, —OR⁹, —C(O)R⁹, —C(O)OR⁹ and —S(O)ₗR⁹; and wherein R⁹ is selected from hydrogen; hydrocarbyl optionally substituted with 1, 2, 3, 4 or 5 R¹⁰; and —(CH₂)ₖ-heterocyclyl optionally substituted with 1, 2, 3, 4 or 5 R¹⁰.

In one embodiment, Z is a bond or is selected from the following linkers:
—Z¹—;
—Z¹—Z²—Z³—;
—Z¹—Z²—Z³—Z⁴—;
—Z¹—Z²—Z³—Z⁴—Z⁵—;
—Z¹—Z²—Z³—Z⁴—Z⁵—Z⁶—;
—Z¹—Z²—Z³—Z⁴—Z⁵—Z⁶—Z⁷—; and
—Z¹—Z²—Z³—Z⁴—Z⁵—Z⁶—Z⁷—Z⁸—;
   wherein Z¹, Z², Z³, Z⁴, Z⁵, Z⁶, Z⁷ and Z⁸ are each independently selected from —O—, —C(O)—, —S(O)ₗ—, —N(R⁸)—, hydrocarbylene (e.g. C₁₋₆ alkylene or C₂₋₆ alkenylene) optionally substituted with 1, 2, 3, 4 or 5 R¹⁰, and heterocyclylene optionally substituted with 1, 2, 3, 4 or 5 R¹⁰. More usually, Z is —Z¹—, —Z¹—Z²— or —Z¹—Z²—Z³—. Z¹ is often —N(R⁸)—, —C(O), —O— or heterocyclylene optionally substituted with 1, 2, 3, 4 or 5 R¹⁰.

In another embodiment, Z is a bond or a linker comprising 1, 2, 3 or 4 linkages selected from selected from —O—, —C(O)—, —S(O)ₗ—, —N(R⁸)—, —CH₂— and —CH═CH—. The linker typically comprises 1, 2 or 3 in-chain atoms. Thus, Z may be selected from —O—, —C(O)—, —N(R⁸)—, —CH₂—, —N(R⁸)C(O)ₗ—, —N(R⁸)S(O)ₗ—, —C(O)N(R⁶)—, —S(O)ₗN(R⁸)—, —N(R⁸)S(O)ₗN(R⁸)—, —CH₂CH₂—, —CH₂O—, —CH₂CH═CH— and —OCH₂CH═CH—. R⁸ is often hydrogen or C₁₋₆ alkyl optionally substituted with 1, 2, 3, 4 or 5 R¹⁰.

In a further embodiment, Z comprises at least one moiety selected from —N(R⁸)—, —C(O)— and —S(O)ₗ—. Of mention are compounds comprising two or more of said moieties.

In a further embodiment, Z comprises at least one carbocyclylene or heterocyclylene moiety, either of which is optionally substituted with 1, 2, 3, 4 or 5 R¹⁰. Of mention are compounds in which Z comprises at least one heterocyclylene moiety. In certain compounds, —Z—R⁶ is attached to the remainder of the compound via said carbocyclylene or heterocyclylene moiety.

In a further embodiment, Z is attached to the ring shown in formula (I) via a nitrogen atom. Thus, included in the invention are compounds in which Z is attached to said ring via an —N(R⁸)— moiety or via a nitrogen atom present in a heterocyclic moiety.

In a further embodiment, Z comprises an —N(R⁸)C(O)— moiety. In certain compounds, the group —Z—R⁶ is attached to the remainder of the compound via the nitrogen atom of said moiety.

In a further embodiment, Z is a linker selected from —N(R⁸)—, —N(R⁸)C(O)—, —N(R⁸)—C₁₋₆ alkylene- and —N(R⁸)C(O)—C₁₋₆ alkylene-, wherein —Z—R⁶ is attached to the remainder of the compound via the nitrogen atom of said linker and wherein any C₁₋₆ alkylene group is optionally substituted with 1, 2, 3, 4 or 5 R¹⁰. Typically, R⁸ is selected from hydrogen, hydrocarbyl optionally substituted with 1, 2, 3, 4 or 5 R¹⁰, and —(CH₂)ₖ-heterocyclyl optionally substituted with 1, 2, 3, 4 or 5 R¹⁰. By way of example, R⁸ may be selected from hydrogen, C₁₋₆ alkyl (e.g. C₁, C₂, C₃ or C₄ alkyl) optionally substituted with 1, 2, 3, 4 or 5 R¹⁰, —(CH₂)ₖ— carbocyclyl (e.g. cyclopropyl, cyclopropylmethyl or benzyl) optionally substituted with 1, 2, 3, 4 or 5 R¹⁰, and —(CH₂)ₖ-heterocyclyl optionally substituted with 1, 2, 3, 4 or 5 R¹⁰.

In a further embodiment, Z is —N(R⁸)C(O)—, wherein —Z—R⁶ is attached to the remainder of the compound via the nitrogen atom of said linker. Typically, R⁸ is selected from hydrogen, hydrocarbyl optionally substituted with 1, 2, 3, 4 or 5 R¹⁰; and —(CH₂)ₖ-heterocyclyl optionally substituted with 1, 2, 3, 4 or 5 R¹⁰. By way of example, R⁸ may be selected from hydrogen, C₁₋₆ alkyl (e.g. C₁, C₂, C₃ or C₄ alkyl) optionally substituted with 1, 2, 3, 4 or 5 R¹⁰, —(CH₂)ₖ— carbocyclyl (e.g. cyclopropyl, cyclopropylmethyl or benzyl) optionally substituted with 1, 2, 3, 4 or 5 R¹⁰, and —(CH₂)ₖ-heterocyclyl optionally substituted with 1, 2, 3, 4 or 5 R¹⁰.

In a further embodiment, Z is carbocyclylene or heterocyclylene, either of which is optionally substituted with 1, 2, 3, 4 or 5 R¹⁰.

In a further embodiment, Z is heterocyclylene optionally substituted with 1, 2, 3, 4 or 5 R¹⁰. Of mention are compounds in which the heterocyclylene group comprises one or more (e.g. 1, 2, 3 or 4) ring nitrogen atoms and optionally one or more ring —C(O)— moieties.

In a further embodiment, Z comprises (e.g. is) a moiety selected from piperidinylene; pyrrolidin-2-onyl[1,3]oxazinan-2-onylene; tetrahydro-pyrimidin-2-onylene; 5,6,7,8-tetrahydro-naphthalenylene; piperazine-2,5-dionylene; isoindole-1,3-dionylene; 1,4-dihydro-2H-isoquinolin-3-onylene; 2,3-dihydro-isoindol-2-onylene; 3,4-dihydro-2H-isoquinolin-1-onylene; 2H-pyridazin-3-onylene; oxazolidin-2-onylene; imidazolidin-2-onylene; hexahydro-pyrido[1,2-a]pyrazine-1,4-dionylene; hexahydro-pyrrolo-[1,2-a]pyrazin-1,4-dionylene; 5,6,7,8-tetrahydro-pyrido[4,3-d]pyrimidinylene; 5,6-dihydro-8H-[1,2,4]triazolo[4,3-a]pyrazinylene; 5,6-dihydro-8H-[1,2,4]triazolo[1,5-a]pyrazinylene; 6,7-dihydro-5H-[1,2,4]triazolo[4,3-a]pyrazin-8-onylene; 6,7-dihydro-5H-[1,2,4]triazolo[1,5-a]pyrazin-8-onylene; 6,7-dihydro-5H-pyrido[3,4-d]pyrimidin-8-onylene; 6,7-dihydro-4H-oxazolo[5,4-c]pyridinylene; 7,8-dihydro-6H-pyrido[4,3-d]pyrimidin-5-onylene; 6H-pyrido[4,3-d]pyrimidin-5-onylene; 5,8-dihydro-6H-pyrido[3,4-d]pyrimidinylene; 7,8-dihydro-[1,2,4]triazolo[4,3-c]pyrimidinylene; and 7,8-dihydro-[1,2,4]triazolo[4,3-a]pyrazin-6-onylene; any of which is optionally substituted with 1, 2, 3, 4 or 5 R¹⁰.

In a further embodiment, Z comprises (e.g. is) a moiety selected from 2H-pyridazin-3-onylene; oxazolidin-2-onylene; imidazolidin-2-onylene; 5,6-dihydro-8H-[1,2,4]triazolo[4,3-a]pyrazinylene; and 6,7-dihydro-5H-[1,2,4]triazolo[4,3-a]pyrazin-8-onylene; any of which is optionally substituted with 1, 2, 3, 4 or 5 R¹⁰.

In a further embodiment, Z comprises (e.g. is) a moiety selected from imidazolidin-2-onylene and pyridazin-3-onylene, either of which is optionally substituted with 1, 2, 3, 4 or 5 R¹⁰.

R⁶ is selected from hydrogen, except when Y and Z are each a bond; hydrocarbyl optionally substituted with 1, 2, 3, 4 or 5 R¹⁰; and —(CH₂)ₖ-heterocyclyl optionally substituted with 1, 2, 3, 4 or 5 R¹⁰.

In one embodiment, R⁶ is hydrogen.

In another embodiment, R⁶ is hydrocarbyl optionally substituted with 1, 2, 3, 4 or 5 R¹⁰. In this case, R⁶ is often selected from C₁₋₆ alkyl (e.g. C₁, C₂, C₃ or C₄ alkyl) or —(CH₂)ₖ— carbocyclyl (e.g. —(CH₂)ₖ-cycloalkyl or —(CH₂)ₖ-aryl), either of which is optionally substituted with 1, 2, 3, 4 or 5 R¹⁰. In particular, R⁶ may be C₁₋₆ alkyl (e.g. C₁, C₂, C₃ or C₄ alkyl), —(CH₂)ₖ-cycloalkyl (e.g. cyclopropyl or cyclopropylmethyl) or —(CH₂)ₖ-aryl (e.g. phenyl or benzyl), any of which is optionally substituted with 1, 2, 3, 4 or 5 R¹⁰.

In a further embodiment, R⁶ is —(CH₂)ₖ-heterocyclyl optionally substituted with 1, 2, 3, 4 or 5 R¹⁰. Typically, k is 0 or 1, more usually 0. The heterocyclyl group may be heterocycloalkyl or heteroaryl, either of which is optionally substituted with 1, 2, 3, 4 or 5 R¹⁰. The heterocyclyl group may be monocyclic or bicyclic, usually monocyclic. Exemplary heterocyclyl groups include oxiranyl, azirinyl, 1,2-oxathiolanyl, imidazolyl, thienyl, furyl, tetrahydrofuryl, pyranyl, thiopyranyl, thianthrenyl, isobenzofuranyl, benzofuranyl, chromenyl, 2H-pyrrolyl, pyrrolyl, pyrrolinyl, pyrrolidinyl, imidazolyl, imidazolidinyl, benzimidazolyl, pyrazolyl, pyrazinyl, pyrazolidinyl, thiazolyl, isothiazolyl, dithiazolyl, oxamlyl, isoxazolyl, pyridyl, pyrazinyl, pyrimidinyl, piperidyl, piperazinyl, pyridazinyl, morpholinyl, thiomorpholinyl, especially thiomorpholino, indolizinyl, isoindolyl, 3H-indolyl, indolyl, benzimidazolyl, cumaryl, indazolyl, triazolyl, tetrazolyl, purinyl, 4H-quinolizinyl, isoquinolyl, quinolyl; tetrahydroquinolyl, tetrahydroisoquinolyl, decahydroquinolyl, octahydroisoquinolyl, benzofuranyl, dibenzofuranyl, benzothiophenyl, dibenzothiophenyl, phthalazinyl, naphthyridinyl, quinoxalyl, quinazolinyl, quinazolinyl, cinnolinyl, pteridinyl, carbazolyl, β-carbolinyl, phenanthridinyl, acridinyl, perimidinyl, phenanthrolinyl, furazanyl, phenazinyl, phenothiazinyl, phenoxazinyl, chromenyl, isochromanyl and chromanyl, any of which is optionally substituted with 1, 2, 3, 4 or 5 R¹⁰.

In a further embodiment, R⁶ is 5,6-dihydro-8H-[1,2,4]triazolo[4,3-a]pyrazin-7-yl, which may be substituted at the 3-position by, for example, trifluoromethyl.

In a further embodiment, R⁶ is carbocyclyl or heterocyclyl, either of which is optionally substituted with 1, 2, 3, 4 or 5 R¹⁰.

In a further embodiment, R⁶ is aryl or heteroaryl, either of which is optionally substituted with 1, 2, 3, 4 or 5 R¹⁰.

In a further embodiment, R⁶ is aryl, in particular phenyl or naphthyl, either of which is optionally substituted with 1, 2, 3, 4 or 5 R¹⁰. In embodiments, R⁶ is phenyl optionally substituted with 1, 2, 3, 4 or 5 R¹⁰, wherein the or each R¹⁰ is, for example, hydroxy, halogen (for example, chlorine or fluorine); C₁, C₂, C₃ or C₄ alkyl, for example methyl, ethyl, propyl, isopropyl, n-butyl, sec-butyl or tert-butyl, any of which is optionally substituted with 1, 2, 3 or 4 halogen (e.g. fluorine or chlorine) atoms, an example being trifluoromethyl; or C₁, C₂, C₃ or C₄ alkoxy, for example methoxy, ethoxy, propoxy, isopropoxy, butoxy, tert-butoxy, any of which is optionally substituted with 1, 2, 3 or 4 halogen (e.g. fluorine or chlorine) atoms. For example, R⁵ may be phenyl optionally substituted with 1, 2, 3, 4 or 5 halogen (e.g. fluorine) atoms.

In a further embodiment, R⁶ is heteroaryl (often monocyclic), for example, thienyl or benzothiophenyl, and is optionally substituted with 1, 2, 3, 4 or 5 R¹⁰, wherein the or each R¹⁰ is, for example, hydroxy, halogen (for example, chlorine or fluorine); C₁, C₂, C₃ or C₄ alkyl, for example methyl, ethyl, propyl, isopropyl, n-butyl, sec-butyl or tert-butyl, any of which is optionally substituted with 1, 2, 3 or 4 halogen (e.g. fluorine or chlorine) atoms, an example being trifluoromethyl; or C₁, C₂, C₃ or C₄ alkoxy, for example methoxy, ethoxy, propoxy, isopropoxy, butoxy, tert-butoxy, any of which is optionally substituted with 1, 2, 3 or 4 halogen (e.g. fluorine or chlorine) atoms.

In further embodiment, Z is a bond or a linker comprising 1, 2, 3 or 4 linkages selected from selected from —O—, —C(O)—, —S(O)ₗ—, —N(R⁸)—, —CH₂— and —CH═CH—; and R⁶ is hydrogen or is selected from C₁₋₆ alkyl, cycloalkyl, aryl (e.g. phenyl) and heterocyclyl, any of which is optionally substituted with 1, 2, 3, 4 or 5 R¹⁰.

In a further embodiment, Z is selected from —O—, —O—C₁₋₆ alkylene- and —O—C₁₋₆ alkenylene-; and R⁶ is hydrogen or is selected from C₁₋₆ alkyl, cycloalkyl, aryl (e.g. phenyl) and heterocyclyl, any of which is optionally substituted with 1, 2, 3, 4 or 5 R¹⁰.

In a further embodiment, —Z—R⁶ is selected from R¹⁴, —OR¹⁴, —C(O)R¹⁴, —C(O)OR¹⁴, —C(O)N(R¹⁵)R¹⁶, —N(R¹⁵)R¹⁶, —N(R¹⁵)C(O)R¹⁴, —N(R¹⁵)S(O)ₗR¹⁵, —S(O)ₗR¹⁵ and —S(O)ₗN(R¹⁵)R¹⁶; wherein R¹⁴ is hydrogen or is selected from hydrocarbyl and —(CH₂)ₖ-heterocyclyl, either of which is optionally substituted with 1, 2, 3, 4 or 5 R¹⁰; and wherein R¹⁵ and R¹⁶ are each independently selected from R⁹, —OR⁹, —C(O)R⁹, —C(O)OR⁹ and —S(O)ₗR⁹; or R¹⁵ and R¹⁶ taken together with a nitrogen atom to which they are attached form heterocyclyl optionally substituted with 1, 2, 3, 4 or 5 R¹⁰.

In a further embodiment, R¹⁴, R¹⁵ and R¹⁶ are each independently selected from hydrogen; C₁₋₆ (e.g. C₁, C₂, C₃ or C₄) alkyl optionally substituted with 1, 2, 3, 4 or 5 R¹⁰; and —(CH₂)ₖ-aryl (e.g. phenyl or benzyl) optionally substituted with 1, 2, 3, 4 or 5 R¹⁰.

In a further embodiment, —Z—R⁶ is hydroxy or aliphatic hydrocarbyloxy (e.g. C₁₋₆ alkoxy or C₂₋₆ alkenyloxy). In a particular embodiment, Z is —OCH₂CH═CH— and R⁶ is a 3- to 10- (e.g. 5- or 6-) membered saturated or unsaturated cyclic group, in particular aryl (e.g. phenyl or napthyl), which is optionally substituted with 1, 2, 3, 4 or 5 R¹⁰.

In a further embodiment, —Z—R⁶ comprises at least one carbocyclic or heterocyclic moiety, either of which is optionally substituted with 1, 2, 3, 4 or 5 R¹⁰. In particular embodiments, —Z—R⁶ comprises at least two such moieties, which may be the same or different. By way of example, the or each moiety may be independently selected from cycloalkyl (e.g. cyclopropyl, cyclobutyl, cyclopentyl or cyclohexyl), aryl (e.g. phenyl or naphthyl) and heterocyclyl (e.g. [1, 2,4]triazolo[4,3-a]pyrazinyl, piperidinyl, piperazinyl, pyrrolidinyl, furyl, pyrimidinyl, pyrazinyl, benzimidazolyl, 3,4-dihydroisoquinolinyl, azepanyl, diazepanyl, triazolyl, morpholinyl, pyrazolyl, pyradizinyl, benzofuryl, pyridinyl, isoxazolyl, thiadiazolyl, thiophenyl, imidazo[2,1-b][1,3]thiazolyl, 3,4,6,7-tetrahydro-5H-imida[4,5-c]pyridin-5-yl), any of which is optionally substituted with 1, 2, 3, 4 or 5 R¹⁰.

In a further embodiment, Z is a bond and R⁶ is carbocyclyl or heterocyclyl, either of which is optionally substituted with 1, 2, 3, 4 or 5 R¹⁰. In a particular embodiment, Z is a bond and R⁶ is heterocyclyl optionally substituted with 1, 2, 3, 4 or 5 R¹⁰. Of mention are compounds in which R⁶ comprises one or more (e.g. 1, 2, 3 or 4) ring nitrogen atoms and optionally one or more ring —C(O)— moieties. In certain compounds, R⁶ is attached to the remainder of the compound via a ring nitrogen atom.

In a further embodiment, Z is a bond and R⁶ is selected from piperidinyl; pyrrolidin-2-onyl[1,3]oxazinan-2-onyl; tetrahydro-pyrimidin-2-onyl; 5,6,7,8-tetrahydro-naphthalenyl; piperazine-2,5-dionyl; isoindole-1,3-dionyl; 1,4-dihydro-2H-isoquinolin-3-onyl; 2,3-dihydro-isoindol-2-onyl; 3,4-dihydro-2H-isoquinolin-1-onyl; 2H-pyridazin-3-onyl; oxazolidin-2-onyl; imidazolidin-2-onyl; hexahydro-pyrido[1,2-a]pyrazine-1,4-dionyl; hexahydro-pyrrolo-[1,2-a]pyrazin-1,4-dionyl; 5,6,7,8-tetrahydro-pyrido[4,3-d]pyrimidinyl; 5,6-dihydro-8H-[1,2,4]triazolo[4,3-a]pyrazinyl; 5,6-dihydro-8H-[1,2,4]triazolo[1,5-a]pyrazinyl; 6,7-dihydro-5H-[1,2,4]triazolo[4,3-a]pyrazin-8-onyl; 6,7-dihydro-5H-[1,2,4]triazolo[1,5-a]pyrazin-8-onyl; 6,7-dihydro-5H-pyrido[3,4-d]pyrimidin-8-onyl; 6,7-dihydro-4H-oxazolo[5,4-c]pyridinyl; 7,8-dihydro-6H-pyrido[4,3-d]pyrimidin-5-onyl; 6H-pyrido[4,3-d]pyrimidin-5-onyl; 5,8-dihydro-6H-pyrido[3,4-d]pyrimidinyl; 7,8-dihydro-[1,2,4]triazolo[4,3-c]pyrimidinyl; and 7,8-dihydro-[1,2,4]triazolo[4,3-a]pyrazin-6-onyl; any of which is optionally substituted with 1, 2, 3, 4 or 5 R¹⁰.

In a further embodiment, Z is a bond and R⁶ is selected from 2H-pyridazin-3-onyl; oxazolidin-2-onyl; imidazolidin-2-onyl; 5,6-dihydro-8H-[1,2,4]triazolo[4,3-a]pyrazinyl; and 6,7-dihydro-5H-[1,2,4]triazolo[4,3-a]pyrazin-8-onyl; any of which is optionally substituted with 1, 2, 3, 4 or 5 R¹⁰.

In a further embodiment, Z is a bond and R⁶ is imidazolidin-2-onyl or pyridazin-3-onyl, either of which is optionally substituted with 1, 2, 3, 4 or 5 R¹⁰.

In a further embodiment, Z is a linker selected from —N(R⁸)—, —N(R⁸)C(O)—, —N(R⁸)—C₁₋₆ alkylene- and —N(R⁸)C(O)—C₁₋₆ alkylene-, wherein —Z—R⁶ is attached to the remainder of the compound via the nitrogen atom of said linker and wherein any C₁₋₆ alkylene group is optionally substituted with 1, 2, 3, 4 or 5 R¹⁰; and R⁶ is carbocyclyl or heterocyclyl, either of which is optionally substituted with 1, 2, 3, 4 or 5 R¹⁰. Of mention are compounds in which R⁶ is aryl (e.g. phenyl) or heterocyclyl (e.g. pyridinyl, benzimidazolyl, benzotriazolyl, indazolyl, pyridazinyl or pyrimidinyl), either of which is optionally substituted with 1, 2, 3, 4 or 5 R¹⁰. In particular compounds, R⁶ phenyl or pyridinyl, either of which is optionally substituted with 1, 2, 3, 4 or 5 R¹⁰. In other compounds, R⁶ is substituted by 1, 2, 3, 4 or 5 R¹⁰, at least one of which is carbocyclyl or heterocyclyl, either of which is optionally substituted with 1, 2, 3, 4 or 5 substituents selected from halogen, cyano, amino, hydroxy, C₁₋₆ alkyl and C₁₋₆ alkoxy. By way of example, said at least one R¹⁰ may be selected from cycloalkyl (e.g. cyclopropyl), aryl (e.g. phenyl), heterocycloalkyl (e.g. piperidinyl) and heteroaryl (e.g. pyridinyl), any of which is optionally substituted with 1, 2, 3, 4 or 5 substituents selected from halogen, cyano, amino, hydroxy, C₁₋₆ alkyl and C₁₋₆ alkoxy.

In a further embodiment, Z is —N(R⁸)C(O)—, wherein the group —Z—R⁶ is attached to the remainder of the compound via the nitrogen atom of said linker; and R⁶ is carbocyclyl or heterocyclyl, either of which is optionally substituted with 1, 2, 3, 4 or 5 R¹⁰.

In a further embodiment, Z and R⁶ each independently comprise a carbocyclic or heterocyclic group, and are each optionally substituted with 1, 2, 3, 4 or 5 R¹⁰. Included are compounds of this type in which Z comprises (e.g. is) a heterocyclylene moiety optionally substituted with 1, 2, 3, 4 or 5 R¹⁰; and R⁶ is carbocyclyl or heterocyclyl, either of which is optionally substituted with 1, 2, 3, 4 or 5 R¹⁰. Of mention are compounds in which Z comprises (e.g. is) a moiety selected from 2H-pyridazin-3-onylene, oxazolidin-2-onylene, imidazolidin-2-onylene, 5,6-dihydro-8H-[1,2,4]triazolo[4,3-a]pyrazinylene and 6,7-dihydro-5H-[1,2,4]triazolo[4,3-a]pyrazin-8-onylene, any of which is optionally substituted with 1, 2, 3, 4 or 5 R¹⁰. Exemplary R⁶ groups include aryl (e.g. phenyl) and heteroaryl (e.g. pyridyl, pyrimidinyl, indolyl, quinolinyl, pyrazolyl, triazolyl or thiophenyl) groups, either of which are optionally substituted with 1, 2, 3, 4 or 5 R¹⁰.

### R⁷

R⁷ is present when m is 1, 2, 3, 4, 5 or 6 and may be an R¹⁶ moiety, wherein R¹⁶ is independently selected from halogen, trifluoromethyl, cyano, nitro, oxo, ═NR¹¹, —OR¹¹, —C(O)R¹¹, —C(O)OR¹¹, —OC(O)R¹¹, —S(O)ₗR¹¹, —N(R¹¹)R¹², —C(O)N(R¹¹)R¹², —S(O)ₗN(R¹¹)R¹² and R¹³; wherein R¹¹ and R¹² are each independently hydrogen or R¹³; and R¹³ is selected from hydrocarbyl and —(CH₂)ₖ-heterocyclyl, either of which is optionally substituted with 1, 2, 3, 4 or 5 substituents independently selected from halogen, cyano, amino, hydroxy, C₁₋₆ alkyl and C₁₋₆ alkoxy. Alternatively, an R⁷ moiety and Y taken together with the atom(s) to which they are attached may form carbocyclyl or heterocyclyl, either of which is optionally substituted with 1, 2, 3, 4 or 5 R¹⁰; or two R⁷ moieties taken together may form a bridge between the atoms to which they are attached, wherein the bridge is a hydrocarbylene or —(CH₂)ᵢ—O—(CH₂)ⱼ— bridge, and wherein i and j are each independently 0, 1 or 2.

R⁷ may be attached to a ring carbon or nitrogen atom of the ring shown in Formula (I). When R⁷ is attached to a ring nitrogen atom, it is usually selected from —C(O)R¹¹, —C(O)OR¹¹, —S(O)ₗR¹¹, —C(O)N(R¹¹)R¹², —S(O)ₗN(R¹¹)R¹² and R¹³.

In one embodiment, R⁷ is independently selected from hydrogen, halogen (e.g. fluorine, chlorine or bromine), hydroxy, cyano, amino, —C(O)OH, C₁₋₆ alkyl, C₁₋₆ alkoxy (e.g. C₁, C₂, C₃ or C₄ alkoxy), —C(O)—C₁₋₆ alkyl, —C(O)O—C₁₋₆ alkyl, —S(O)ₗ—C₁₋₆ alkyl, —NH(C₁₋₆ alkyl) and —N(C₁₋₆ alkyl)₂, wherein any C₁₋₆ alkyl group present is optionally substituted with 1, 2, 3, 4 or 5 substituents independently selected from halogen, cyano, amino, hydroxy and C₁₋₆ alkoxy.

In another embodiment, R⁷ is independently selected from halogen (e.g. fluorine or chlorine), cyano, amino, hydroxy, C₁₋₆ alkyl (e.g. C₁, C₂, C₃ or C₄ alkyl) and C₁₋₆ alkoxy (e.g. C₁, C₂, C₃ or C₄ alkoxy), any C₁₋₆ alkyl group present is optionally substituted with 1, 2, 3, 4 or 5 substituents independently selected from halogen, cyano, amino, hydroxy and C₁₋₆ alkoxy.

In a further embodiment, m is 0, 1 or 2.

In a further embodiment, m is 0 or 1.

In a further embodiment, m is 1.

In a further embodiment, m is 0.

### R¹⁰

Each R¹⁰ is independently selected from halogen, trifluoromethyl, cyano, nitro, oxo, ═NR¹¹, —OR¹¹, —C(O)R¹¹, —C(O)OR¹¹, —OC(O)R¹¹, —S(O)ₗR¹¹, —N(R¹¹)R¹², C(O)N(R¹¹)R¹², —S(O)ₗN(R¹¹)R¹² and R¹¹; wherein R¹¹ and R¹² are each independently hydrogen or R¹³; and R¹³ is selected from hydrocarbyl and —(CH₂)ₖ-heterocyclyl, either of which is optionally substituted with 1, 2, 3, 4 or 5 substituents independently selected from halogen, cyano, amino, hydroxy, C₁₋₆ alkyl and C₁₋₆ alkoxy.

Typically, each R¹⁰ is independently selected from halogen (e.g. fluorine, chlorine or bromine), hydroxy, cyano, amino, —C(O)OH, C₁₋₆ alkyl, C₁₋₆ alkoxy (e.g. C₁, C₂, C₃ or C₄ alkoxy), —C(O)—C₁₋₆ alkyl, —C(O)O—C₁₋₆ alkyl, —S(O)ₗ—C₁₋₆ alkyl, —NH(C₁₋₆ alkyl) and —N(C₁₋₆ alkyl)₂, wherein any C₁₋₆ alkyl group present is optionally substituted with 1, 2, 3, 4 or 5 substituents independently selected from halogen, cyano, amino, hydroxy and C₁₋₆ alkoxy.

For the avoidance of doubt, where a group is substituted with more than one R¹⁰, each R¹⁰ is independently selected from the range of substituents specified. The same applies to compounds of the invention comprising more than one R¹⁰ substituent; each R¹⁰ is selected independently of any other R¹⁰ substituent present in the compound. As previously indicated, where R¹⁰ is halo, particularly fluoro, any number of hydrogens may in principle be replaced.

Of mention is a compound of the following Formula:

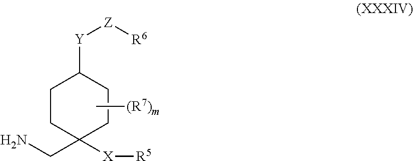

or a pharmaceutically acceptable salt or prodrug thereof.

Also of mention is a compound of the following Formula:

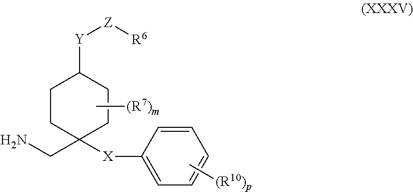

wherein p is as defined elsewhere herein;
or a pharmaceutically acceptable salt or prodrug thereof.

Also of mention is a compound of the following Formula:

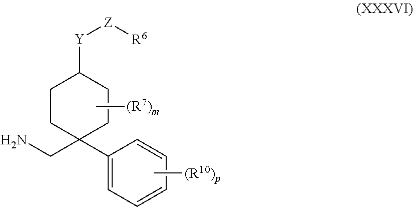

or a pharmaceutically acceptable salt or prodrug thereof.

Of particular mention is a compound of the following Formula:

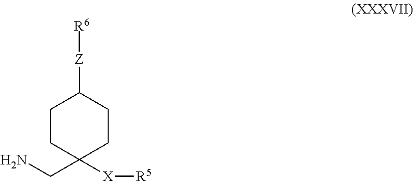

or a pharmaceutically acceptable salt or prodrug thereof.

Also of mention is a compound of the following Formula:

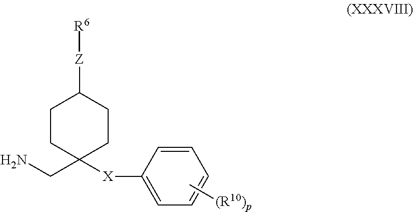

or a pharmaceutically acceptable salt or prodrug thereof.

Also of mention is a compound of the following Formula:

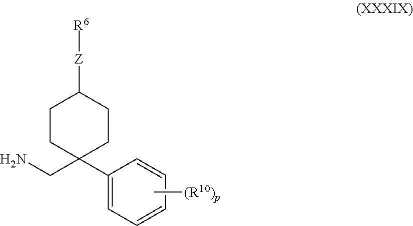

or a pharmaceutically acceptable salt or prodrug thereof.

Also of mention is a compound of the following Formula:

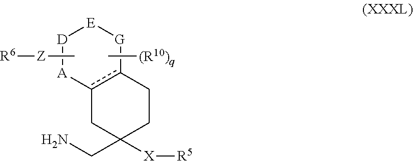

wherein A, D, E, G and q are as defined elsewhere herein;
or a pharmaceutically acceptable salt or prodrug thereof.

The invention therefore includes compounds of the following Formulae:

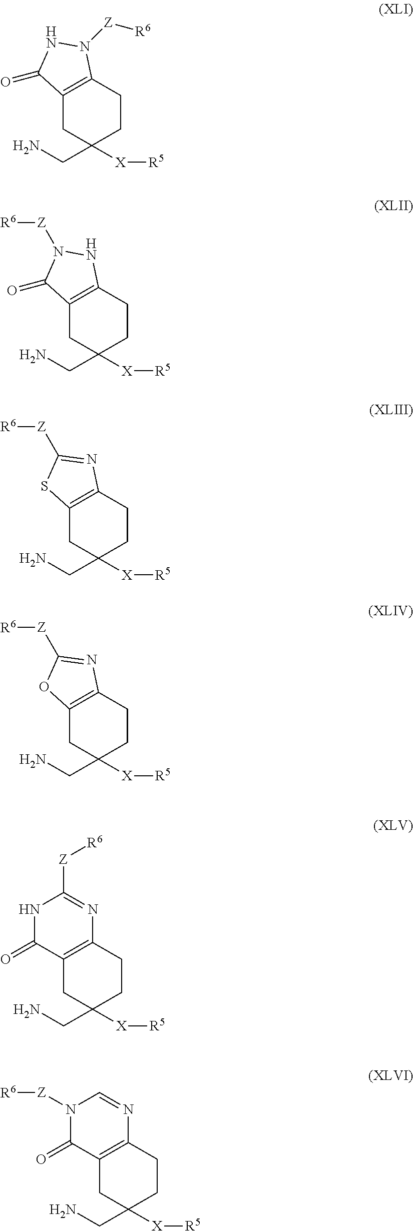

or, in each case, a pharmaceutically acceptable salt or prodrug thereof.

Also of mention is a compound of the following Formula:

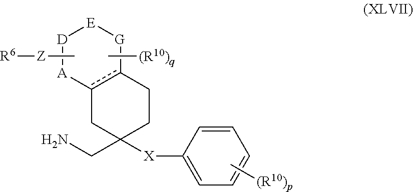

wherein p is as defined elsewhere herein;
   or a pharmaceutically acceptable salt or prodrug thereof.

The invention therefore includes compounds of the following Formulae:

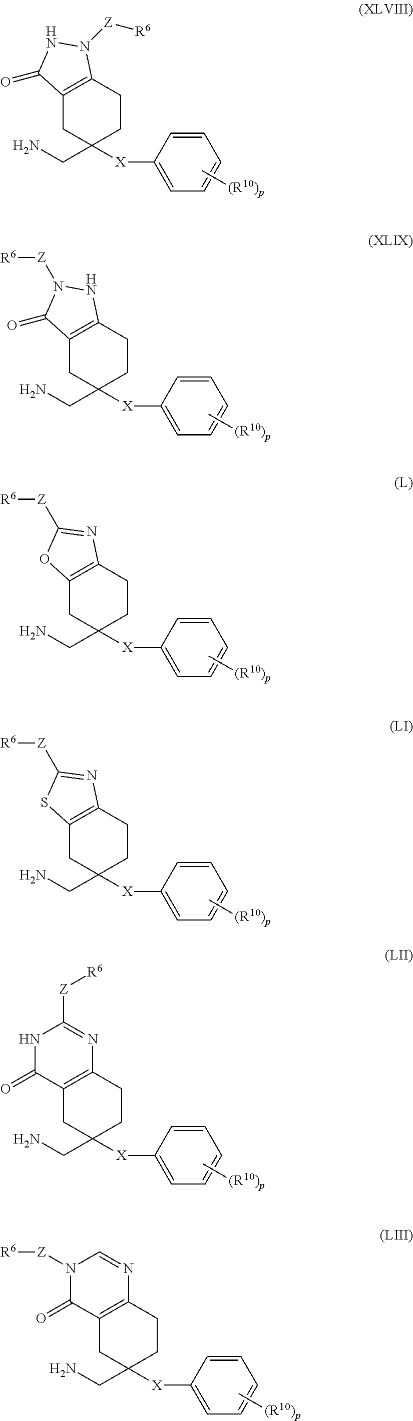

or, in each case, a pharmaceutically acceptable salt or prodrug thereof.

Also of mention is a compound of the following Formula:

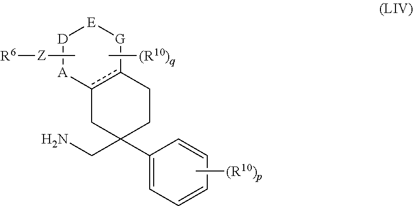

or a pharmaceutically acceptable salt or prodrug thereof.

The invention therefore includes compounds of the following Formulae:

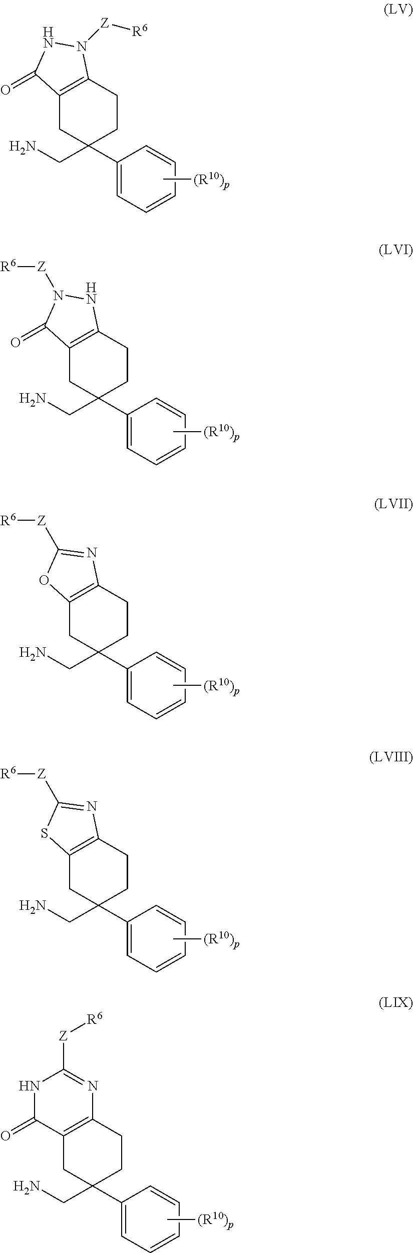

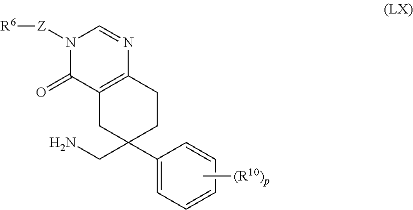

or, in each case, a pharmaceutically acceptable salt or prodrug thereof.

Also of mention is a compound of the following Formula:

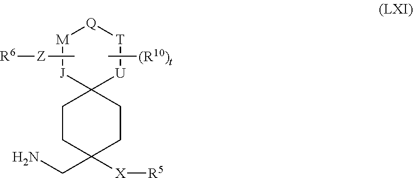

wherein J, M, Q, T, U and t are as defined elsewhere herein;
or a pharmaceutically acceptable salt or prodrug thereof.

The invention therefore includes compounds of the following Formulae:

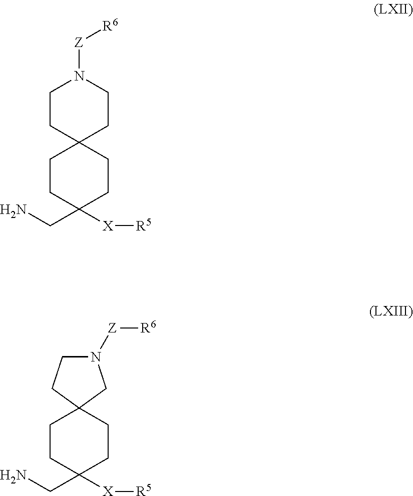

or, in each case, a pharmaceutically acceptable salt or prodrug thereof.

Also of mention is a compound of the following Formula:

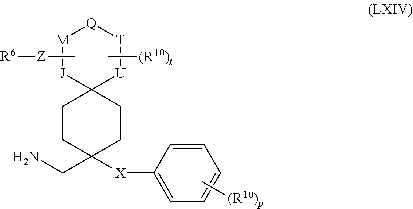

wherein p is as defined elsewhere herein;
or a pharmaceutically acceptable salt or prodrug thereof.

The invention therefore includes compounds of the following Formulae:

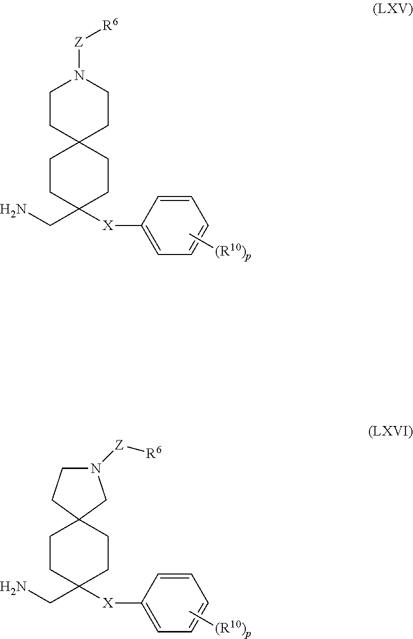

or, in each case, a pharmaceutically acceptable salt or prodrug thereof.

Also of mention is a compound of the following Formula:

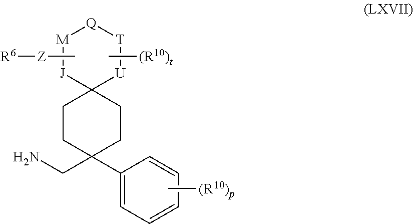

or a pharmaceutically acceptable salt or prodrug thereof.

The invention therefore includes compounds of the following Formulae:

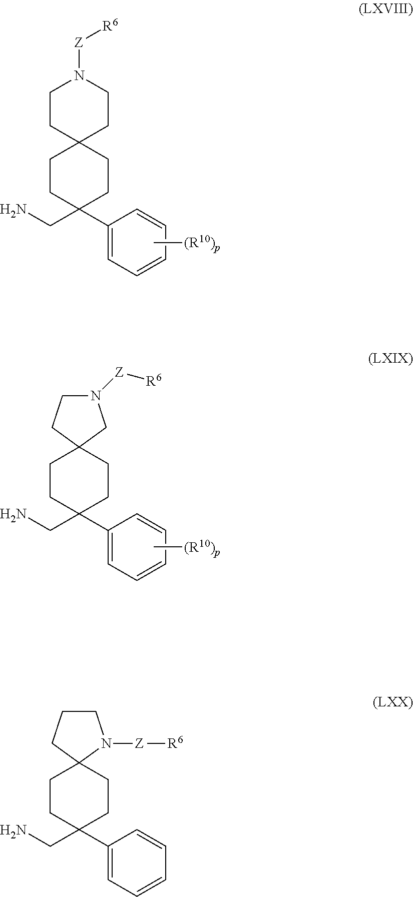

or, in each case, a pharmaceutically acceptable salt or prodrug thereof.

With regard to Formulae (XXXI) to (LXX), Z may be a bond or a linker comprising 1 to 12 in-chain atoms. For example, Z may comprise 1, 2, 3 or 4 linkages selected from selected from —O—, —C(O)—, —S(O)ₗ—, —N(R⁸)—, —CH₂— and —CH═CH—; and R⁶ may be hydrogen or selected from C₁₋₆ alkyl, cycloalkyl, aryl (e.g. phenyl) and heterocyclyl, any of which is optionally substituted with 1, 2, 3, 4 or 5 R¹⁰.

In further embodiments of said formulae, Z is selected from —O—, —O—C₁₋₆ alkylene- and 6 alkenylene-; and R⁶ is hydrogen or is selected from C₁₋₆ alkyl, cycloalkyl, aryl (e.g. phenyl) and heterocyclyl, any of which is optionally substituted with 1, 2, 3, 4 or 5 R¹⁰.

In further embodiments, Z comprises at least one moiety selected from —N(R⁸)—, —C(O)— and —S(O)ₗ—. Of mention are compounds comprising two or more of said moieties.

In further embodiments, Z comprises at least one carbocyclylene or heterocyclylene moiety, either of which is optionally substituted with 1, 2, 3, 4 or 5 R¹⁰. Of mention are compounds in which Z comprises at least one heterocyclylene moiety. In certain compounds, —Z—R⁶ is attached to the remainder of the compound via said carbocyclylene or heterocyclylene moiety.

In further embodiments, Z is attached to the ring shown in formula (I) via a nitrogen atom. Thus, included in the invention are compounds in which Z is attached to said ring via an —N(R⁸)— moiety or via a nitrogen atom present in a heterocyclic moiety.

In further embodiments, Z comprises an —N(R⁸)C(O)— moiety. In certain compounds, the group —Z—R⁸ is attached to the remainder of the compound via the nitrogen atom of said moiety.

In further embodiments, Z is a linker selected from —N(R⁸)—, —N(R⁸)C(O)—, —N(R⁸)—C₁₋₆ alkylene- and —N(R⁸)C(O)—C₁₋₆ alkylene-, wherein —Z—R⁸ is attached to the remainder of the compound via the nitrogen atom of said linker and wherein any C₁₋₆ alkylene group is optionally substituted with 1, 2, 3, 4 or 5 R¹⁰. Typically, R⁸ is selected from hydrogen, hydrocarbyl optionally substituted with 1, 2, 3, 4 or 5 R¹⁰, and —(CH₂)ₖ-heterocyclyl optionally substituted with 1, 2, 3, 4 or 5 R¹⁰. By way of example, R⁸ may be selected from hydrogen, C₁₋₆ alkyl (e.g. C₁, C₂, C₃ or C₄ alkyl) optionally substituted with 1, 2, 3, 4 or 5 R¹⁰, —(CH₂)ₖ-carbocyclyl (e.g. cyclopropyl, cyclopropylmethyl or benzyl) optionally substituted with 1, 2, 3, 4 or 5 R¹⁰, and —(CH₂)ₖ-heterocyclyl optionally substituted with 1, 2, 3, 4 or 5 R¹⁰.

In further embodiments, Z is —N(R⁸)C(O)—, wherein —Z—R⁸ is attached to the remainder of the compound via the nitrogen atom of said linker. Typically, R⁸ is selected from hydrogen, hydrocarbyl optionally substituted with 1, 2, 3, 4 or 5 R¹⁰; and —(CH₂)ₖ-heterocyclyl optionally substituted with 1, 2, 3, 4 or 5 R¹⁰. By way of example, R⁸ may be selected from hydrogen, C₁₋₆ alkyl (e.g. C₁, C₂, C₃ or C₄ alkyl) optionally substituted with 1, 2, 3, 4 or 5 R¹⁰, —(CH₂)ₖ-carbocyclyl (e.g. cyclopropyl, cyclopropylmethyl or benzyl) optionally substituted with 1, 2, 3, 4 or 5 R¹⁰, and —(CH₂)ₖ-heterocyclyl optionally substituted with 1, 2, 3, 4 or 5 R¹⁰.

In further embodiments, Z is carbocyclylene or heterocyclylene, either of which is optionally substituted with 1, 2, 3, 4 or 5 R¹⁰.

In further embodiments, Z is heterocyclylene optionally substituted with 1, 2, 3, 4 or 5 R¹⁰. Of mention are compounds in which the heterocyclylene group comprises one or more (e.g. 1, 2, 3 or 4) ring nitrogen atoms and optionally one or more ring —C(O)— moieties.

In further embodiments, Z comprises (e.g. is) a moiety selected from piperidinylene; pyrrolidin-2-onyl[1,3]oxazinan-2-onylene; tetrahydro-pyrimidin-2-onylene; 5,6,7,8-tetrahydro-naphthalenylene; piperazine-2,5-dionylene; isoindole-1,3-dionylene; 1,4-dihydro-2H-isoquinolin-3-onylene; 2,3-dihydro-isoindol-2-onylene; 3,4-dihydro-2H-isoquinolin-1-onylene; 2H-pyridazin-3-onylene; oxazolidin-2-onylene; imidazolidin-2-onylene; hexahydro-pyrido[1,2-a]pyrazine-1,4-dionylene; hexahydro-pyrrolo-[1,2-a]pyrazin-1,4-dionylene; 5,6,7,8-tetrahydro-pyrido[4,3-d]pyrimidinylene; 5,6-dihydro-8H-[1,2,4]triazolo[4,3-a]pyrazinylene; 5,6-dihydro-8H-[1,2,4]triazolo[1,5-a]pyrazinylene; 6,7-dihydro-5H-[1,2,4]triazolo[4,3-a]pyrazin-8-onylene; 6,7-dihydro-5H-[1,2,4]triazolo[1,5-a]pyrazin-8-onylene; 6,7-dihydro-5H-pyrido[3,4-d]pyrimidin-8-onylene; 6,7-dihydro-4H-oxazolo[5,4-c]pyridinylene; 7,8-dihydro-6H-pyrido[4,3-d]pyrimidin-5-onylene; 6H-pyrido[4,3-d]pyrimidin-5-onylene; 5,8-dihydro-6H-pyrido[3,4-d]pyrimidinylene; 7,8-dihydro-[1,2,4]triazolo[4,3-c]pyrimidinylene; and 7,8-dihydro-[1,2,4]triazolo[4,3-a]pyrazin-6-onylene; any of which is optionally substituted with 1, 2, 3, 4 or 5 R¹⁰.

In further embodiments, Z comprises (e.g. is) a moiety selected from 2H-pyridazin-3-onylene; oxazolidin-2-onylene; imidazolidin-2-onylene; 5,6-dihydro-8H-[1,2,4]triazolo[4,3-a]pyrazinylene; and 6,7-dihydro-5H-[1,2,4]triazolo[4,3-a]pyrazin-8-onylene; any of which is optionally substituted with 1, 2, 3, 4 or 5 R¹⁰.

In further embodiments, Z comprises (e.g. is) a moiety selected from imidazolidin-2-onylene and pyridazin-3-onylene, either of which is optionally substituted with 1, 2, 3, 4 or 5 R¹⁰.

In further embodiments, —Z—R⁶ is selected from R¹⁴, —OR¹⁴, —C(O)R¹⁴, —C(O)OR¹⁴, —C(O)N(R¹⁵)R¹⁶, —N(R¹⁵)R¹⁶, —N(R¹⁵)C(O)R¹⁴, —N(R¹⁵)S(O)ₗR¹⁵, —S(O)ₗR¹⁵ and —S(O)ₗN(R¹⁵)R¹⁶; wherein R¹⁴ is hydrogen or is selected from hydrocarbyl and —(CH₂)ₖ-heterocyclyl, either of which is optionally substituted with 1, 2, 3, 4 or 5 R¹⁰; and wherein R¹⁵ and R¹⁶ are each independently selected from R⁹, —OR⁹, —C(O)R⁹, —C(O)OR⁹ and —S(O)ₗR⁹; or R¹⁵ and R¹⁶ taken together with a nitrogen atom to which they are attached form heterocyclyl optionally substituted with 1, 2, 3, 4 or 5 R¹⁰.

In further embodiments, R¹⁴, R¹⁵ and R¹⁶ are each independently selected from hydrogen; C₁₋₆ (e.g. C₁, C₂, C₃ or C₄) alkyl optionally substituted with 1, 2, 3, 4 or 5 R¹⁰; and —(CH₂)ₖ-aryl (e.g. phenyl or benzyl) optionally substituted with 1, 2, 3, 4 or 5 R¹⁰.

In further embodiments, —Z—R⁶ is hydroxy or aliphatic hydrocarbyloxy (e.g. alkoxy or C₂₋₆ alkenyloxy). In a particular embodiment, Z is —OCH₂CH═CH— and R⁶ is a 3- to 10- (e.g. 5- or 6-) membered saturated or unsaturated cyclic group, in particular aryl (e.g. phenyl or napthyl), which is optionally substituted with 1, 2, 3, 4 or 5 R¹⁰.

In further embodiments, —Z—R⁶ comprises at least one carbocyclic or heterocyclic moiety, either of which is optionally substituted with 1, 2, 3, 4 or 5 R¹⁰. In particular embodiments, —Z—R⁶ comprises at least two such moieties, which may be the same or different. By way of example, the or each moiety may be independently selected from cycloalkyl (e.g. cyclopropyl, cyclobutyl, cyclopentyl or cyclohexyl), aryl (e.g. phenyl or naphthyl) and heterocyclyl (e.g. [1,2,4]triazolo[4,3-a]pyrazinyl, piperidinyl, piperazinyl, pyrrolidinyl, furyl, pyrimidinyl, pyrazinyl, benzimidazolyl, 3,4-dihydroisoquinolinyl, azepanyl, diazepanyl, triazolyl, morpholinyl, pyrazolyl, pyradizinyl, benzofuryl, pyridinyl, isoxazolyl, thiadiazolyl, thiophenyl, imidazo[2,1-b][1,3]thiazolyl, 3,4,6,7-tetrahydro-5H-imida[4,5-c]pyridin-5-yl), any of which is optionally substituted with 1, 2, 3, 4 or 5 R¹⁰.

In further embodiments, Z is a bond and R⁶ is carbocyclyl or heterocyclyl, either of which is optionally substituted with 1, 2, 3, 4 or 5 R¹⁰. In a particular embodiment, Z is a bond and R⁶ is heterocyclyl optionally substituted with 1, 2, 3, 4 or 5 R¹⁰. Of mention are compounds in which R⁶ comprises one or more (e.g. 1, 2, 3 or 4) ring nitrogen atoms and optionally one or more ring —C(O)— moieties. In certain compounds, R⁶ is attached to the remainder of the compound via a ring nitrogen atom.

In further embodiments, Z is a bond and R⁶ is selected from piperidinyl; pyrrolidin-2-onyl[1,3]oxazinan-2-onyl; tetrahydro-pyrimidin-2-onyl; 5,6,7,8-tetrahydro-naphthalenyl; piperazine-2,5-dionyl; isoindole-1,3-dionyl; 1,4-dihydro-2H-isoquinolin-3-onyl; 2,3-dihydro-isoindol-2-onyl; 3,4-dihydro-2H-isoquinolin-1-onyl; 2H-pyridazin-3-onyl; oxazolidin-2-onyl; imidazolidin-2-onyl; hexahydro-pyrido[1,2-a]pyrazine-1,4-dionyl; hexahydro-pyrrolo-[1,2-a]pyrazin-1,4-dionyl; 5,6,7,8-tetrahydro-pyrido[4,3-d]pyrimidinyl; 5,6-dihydro-8H-[1,2,4]triazolo[4,3-a]pyrazinyl; 5,6-dihydro-8H-[1,2,4]triazolo[1,5-a]pyrazinyl; 6,7-dihydro-5H-[1,2,4]triazolo[4,3-a]pyrazin-8-onyl; 6,7-dihydro-5H-[1,2,4]triazolo[1,5-a]pyrazin-8-onyl; 6,7-dihydro-5H-pyrido[3,4-d]pyrimidin-8-onyl; 6,7-dihydro-4H-oxazolo[5,4-c]pyridinyl; 7,8-dihydro-6H-pyrido[4,3-d]pyrimidin-5-onyl; 6H-pyrido[4,3-d]pyrimidin-5-onyl; 5,8-dihydro-6H-pyrido[3,4-d]pyrimidinyl; 7,8-dihydro-[1,2,4]triazolo[4,3-c]pyrimidinyl; and 7,8-dihydro-[1,2,4]triazolo[4,3-a]pyrazin-6-onyl; any of which is optionally substituted with 1, 2, 3, 4 or 5 R¹⁰.

In further embodiments, Z is a bond and R⁶ is selected from 2H-pyridazin-3-onyl; oxazolidin-2-onyl; imidazolidin-2-onyl; 5,6-dihydro-8H-[1,2,4]triazolo[4,3-a]pyrazinyl; and 6,7-dihydro-5H-[1,2,4]triazolo[4,3-a]pyrazin-8-onyl; any of which is optionally substituted with 1, 2, 3, 4 or 5 R¹⁰.

In further embodiments, Z is a bond and R⁶ is imidazolidin-2-onyl or pyridazin-3-onyl, either of which is optionally substituted with 1, 2, 3, 4 or 5 R¹⁰.

In further embodiments, Z is a linker selected from —N(R⁸)—, —N(R⁸)C(O)—, —N(R⁸)—C₁₋₆ alkylene- and —N(R⁸)C(O)—C₁₋₆ alkylene-, wherein —Z—R⁶ is attached to the remainder of the compound via the nitrogen atom of said linker and wherein any C₁₋₆ alkylene group is optionally substituted with 1, 2, 3, 4 or 5 R¹⁰; and R⁶ is carbocyclyl or heterocyclyl, either of which is optionally substituted with 1, 2, 3, 4 or 5 R¹⁰. Of mention are compounds in which R⁶ is aryl (e.g. phenyl) or heterocyclyl (e.g. pyridinyl, benzimidazolyl, benzotriazolyl, indazolyl, pyridazinyl or pyrimidinyl), either of which is optionally substituted with 1, 2, 3, 4 or 5 R¹⁰. In particular compounds, R⁶ phenyl or pyridinyl, either of which is optionally substituted with 1, 2, 3, 4 or 5 R¹⁰. In other compounds, R⁶ is substituted by 1, 2, 3, 4 or 5 R¹⁰, at least one of which is carbocyclyl or heterocyclyl, either of which is optionally substituted with 1, 2, 3, 4 or 5 substituents selected from halogen, cyano, amino, hydroxy, C₁₋₆ alkyl and C₁₋₆ alkoxy. By way of example, said at least one R¹⁰ may be selected from cycloalkyl (e.g. cyclopropyl), aryl (e.g. phenyl), heterocycloalkyl (e.g. piperidinyl) and heteroaryl (e.g. pyridinyl), any of which is optionally substituted with 1, 2, 3, 4 or 5 substituents selected from halogen, cyano, amino, hydroxy, C₁₋₆ alkyl and C₁₋₆ alkoxy.

In further embodiments, Z is —N(R⁸)C(O)—, wherein the group —Z—R⁶ is attached to the remainder of the compound via the nitrogen atom of said linker; and R⁶ is carbocyclyl or heterocyclyl, either of which is optionally substituted with 1, 2, 3, 4 or 5 R¹⁰.

In further embodiments, Z and R⁶ each independently comprise a carbocyclic or heterocyclic group, and are each optionally substituted with 1, 2, 3, 4 or 5 R¹⁰. Included are compounds of this type in which Z comprises (e.g. is) a heterocyclylene moiety optionally substituted with 1, 2, 3, 4 or 5 R¹⁰; and R⁶ is carbocyclyl or heterocyclyl, either of which is optionally substituted with 1, 2, 3, 4 or 5 R¹⁰. Of mention are compounds in which Z comprises (e.g. is) a moiety selected from 2H-pyridazin-3-onylene, oxazolidin-2-onylene, imidazolidin-2-onylene, 5,6-dihydro-8H-[1,2,4]triazolo[4,3-a]pyrazinylene and 6,7-dihydro-5H-[1,2,4]triazolo[4,3-a]pyrazin-8-onylene, any of which is optionally substituted with 1, 2, 3, 4 or 5 R¹⁰. Exemplary R⁶ groups include aryl (e.g. phenyl) and heteroaryl (e.g. pyridyl, pyrimidinyl, indolyl, quinolinyl, pyrazolyl, triazolyl or thiophenyl) groups, either of which are optionally substituted with 1, 2, 3, 4 or 5 R¹⁰.

In further embodiments of the above formulae, when p is 1, 2, 3, 4 or 5, at least one R¹⁰ is halogen or C₁₋₆ alkyl. In particular embodiments, the or each R¹⁰ is independently halogen or C₁₋₆ alkyl.

In further embodiments, when p is 1, 2, 3, 4 or 5, at least one R¹⁰ is halogen. In particular embodiments, the or each R¹⁰ is halogen.

In further embodiments, when p is 1, 2, 3, 4 or 5, at least one R¹⁰ is fluorine or chlorine. In particular embodiments, the or each R¹⁰ is independently fluorine or chlorine.

In further embodiments, p is 0, 1, 2 or 3. In particular embodiments, p is 0, 1 or 2.

Examples of compounds of the invention include those shown below. It will of course be appreciated that, where appropriate, each compound may be in the form of the free compound, an acid or base addition salt, or a prodrug. Where a nitrogen atom forming only two bonds is shown, this represents NH.

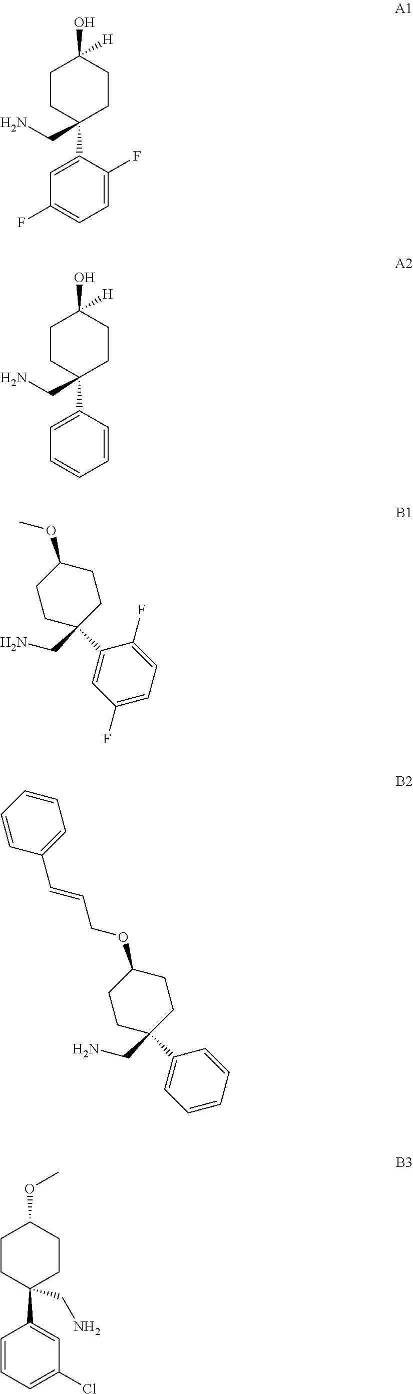 [SMILES: NC[C@]1(c2cc(F)ccc2F)CC[C@H](O)CC1 ; NC[C@]1(c2ccccc2)CC[C@H](O)CC1 ; CO[C@H]1CC[C@](CN)(c2cc(F)ccc2F)CC1 ; NC[C@]1(c2ccccc2)CC[C@H](OC/C=C/c2ccccc2)CC1 ; CO[C@H]1CC[C@](CN)(c2cccc(Cl)c2)CC1]

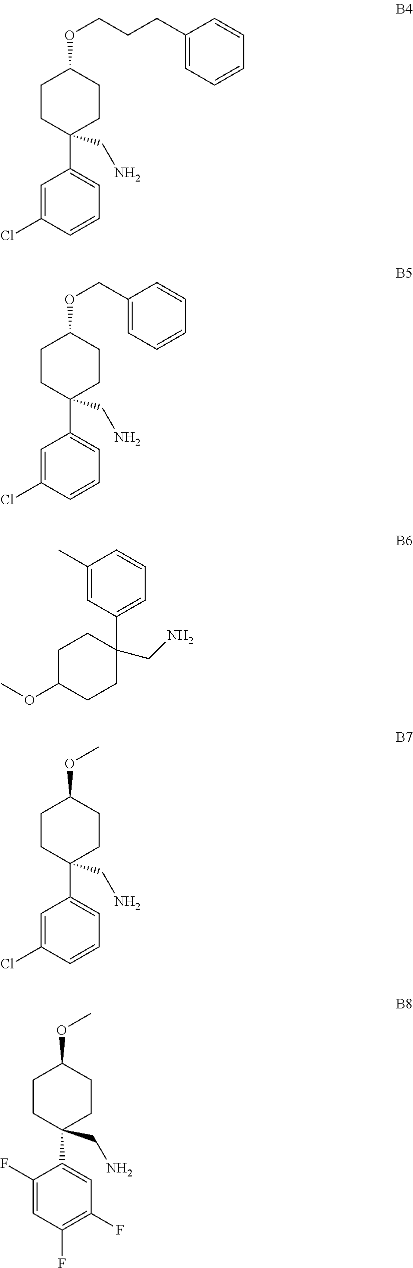 [SMILES: NC[C@]1(c2cccc(Cl)c2)CC[C@H](OCCCc2ccccc2)CC1 ; NC[C@]1(c2cccc(Cl)c2)CC[C@H](OCc2ccccc2)CC1 ; COC1CCC(CN)(c2cccc(C)c2)CC1 ; CO[C@H]1CC[C@@](CN)(c2cccc(Cl)c2)CC1 ; CO[C@H]1CC[C@](CN)(c2cc(F)c(F)cc2F)CC1]

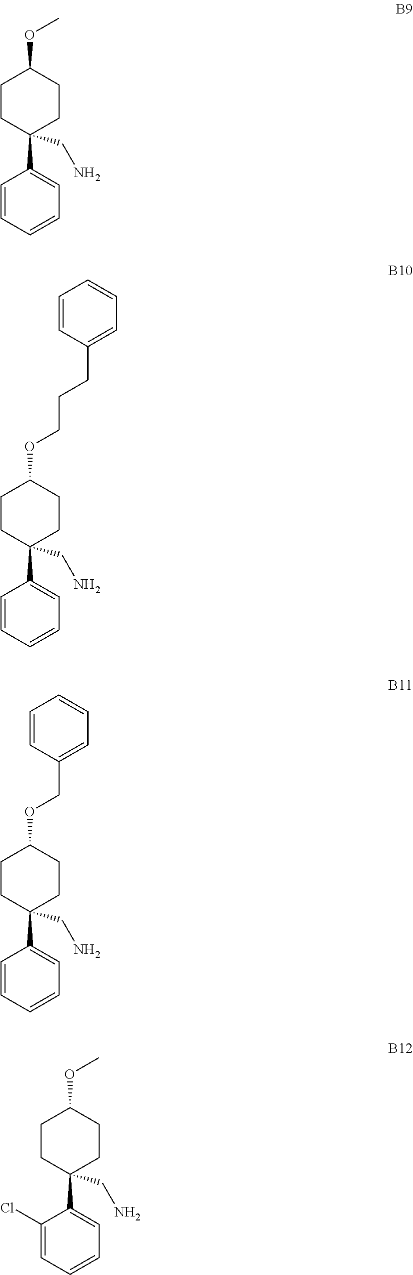 [SMILES: CO[C@H]1CC[C@@](CN)(c2ccccc2)CC1 ; NC[C@]1(c2ccccc2)CC[C@H](OCCCc2ccccc2)CC1 ; NC[C@]1(c2ccccc2)CC[C@H](OCc2ccccc2)CC1 ; CO[C@H]1CC[C@](CN)(c2ccccc2Cl)CC1]

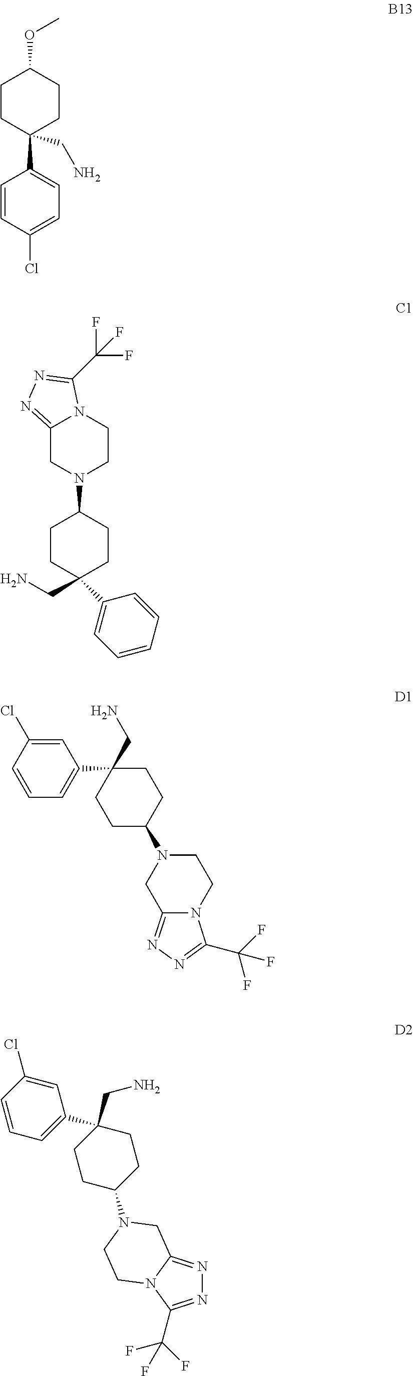 [SMILES: CO[C@H]1CC[C@](CN)(c2ccc(Cl)cc2)CC1 ; NC[C@]1(c2ccccc2)CC[C@H](N2CCn3c(nnc3C(F)(F)F)C2)CC1 ; NC[C@]1(c2cccc(Cl)c2)CC[C@H](N2CCn3c(nnc3C(F)(F)F)C2)CC1 ; NC[C@]1(c2cccc(Cl)c2)CC[C@@H](N2CCn3c(nnc3C(F)(F)F)C2)CC1]

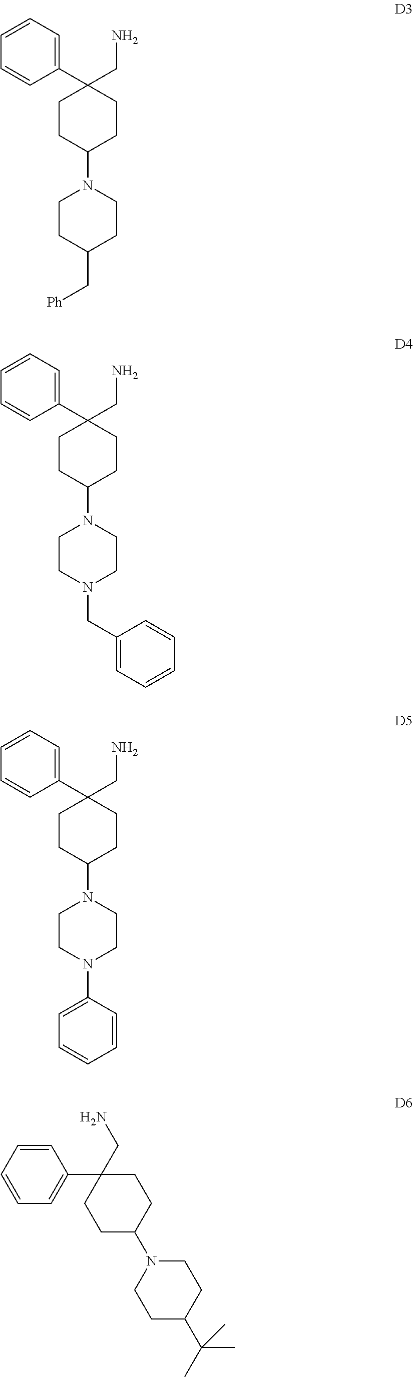 [SMILES: NCC1(c2ccccc2)CCC(N2CCC(Cc3ccccc3)CC2)CC1 ; NCC1(c2ccccc2)CCC(N2CCN(Cc3ccccc3)CC2)CC1 ; NCC1(c2ccccc2)CCC(N2CCN(c3ccccc3)CC2)CC1 ; CC(C)(C)C1CCN(C2CCC(CN)(c3ccccc3)CC2)CC1]

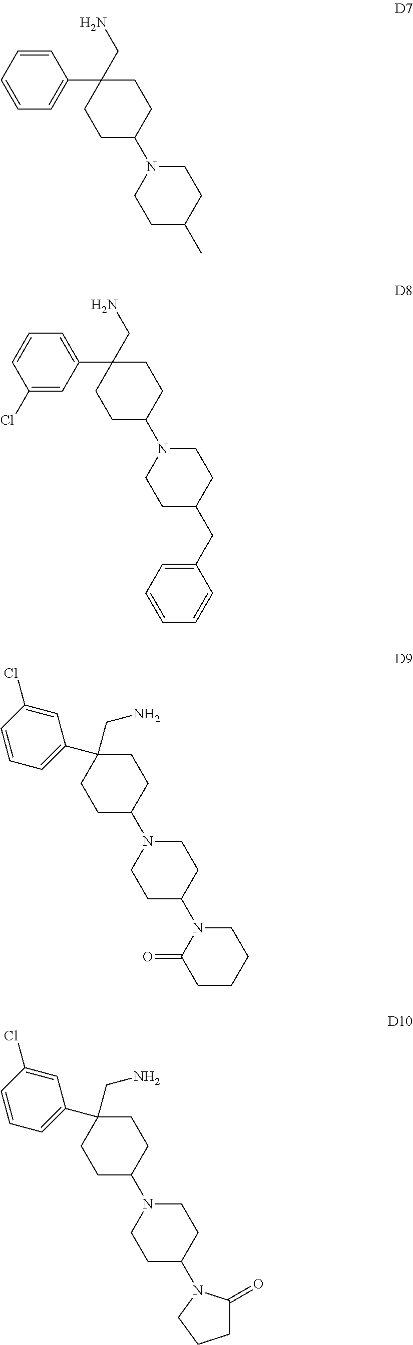 [SMILES: CC1CCN(C2CCC(CN)(c3ccccc3)CC2)CC1 ; NCC1(c2cccc(Cl)c2)CCC(N2CCC(Cc3ccccc3)CC2)CC1 ; NCC1(c2cccc(Cl)c2)CCC(N2CCC(N3CCCCC3=O)CC2)CC1 ; NCC1(c2cccc(Cl)c2)CCC(N2CCC(N3CCCC3=O)CC2)CC1]

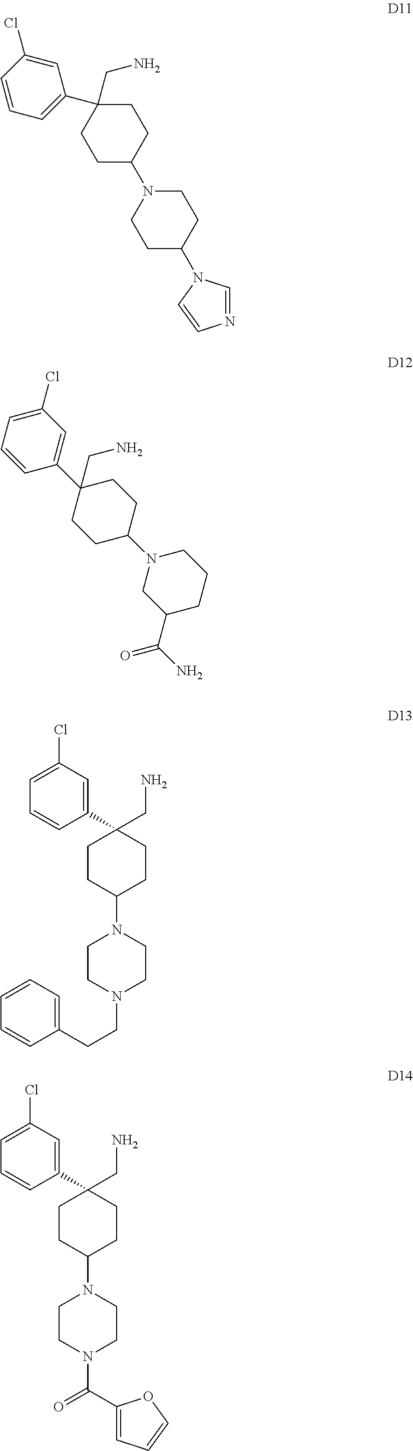

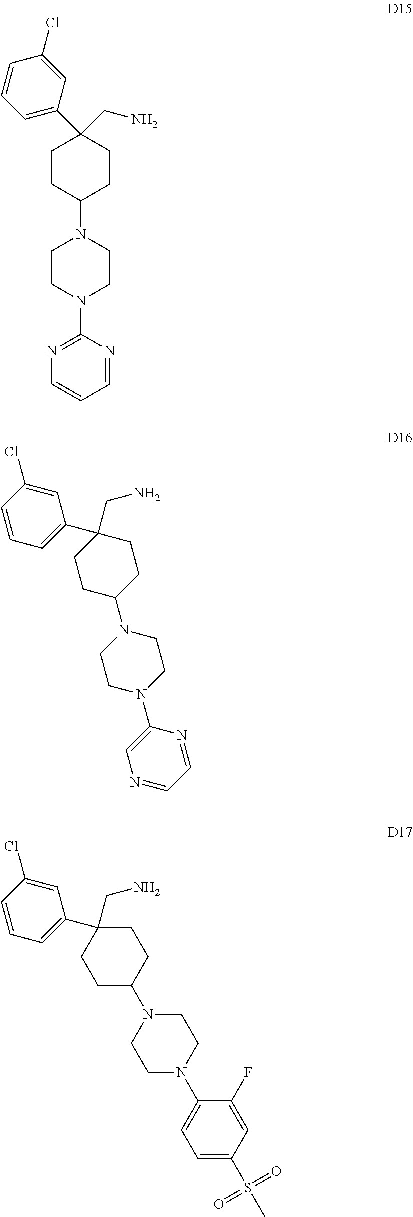 [SMILES: NCC1(c2cccc(Cl)c2)CCC(N2CCN(c3ncccn3)CC2)CC1 ; NCC1(c2cccc(Cl)c2)CCC(N2CCN(c3cnccn3)CC2)CC1 ; CS(=O)(=O)c1ccc(N2CCN(C3CCC(CN)(c4cccc(Cl)c4)CC3)CC2)c(F)c1]

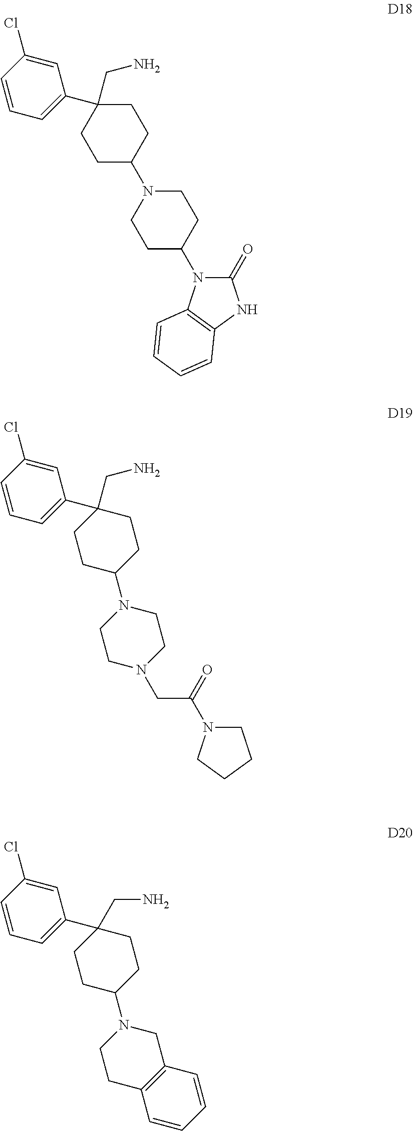 [SMILES: NCC1(c2cccc(Cl)c2)CCC(N2CCC(n3c(=O)[nH]c4ccccc43)CC2)CC1 ; NCC1(c2cccc(Cl)c2)CCC(N2CCN(CC(=O)N3CCCC3)CC2)CC1 ; NCC1(c2cccc(Cl)c2)CCC(N2CCc3ccccc3C2)CC1]

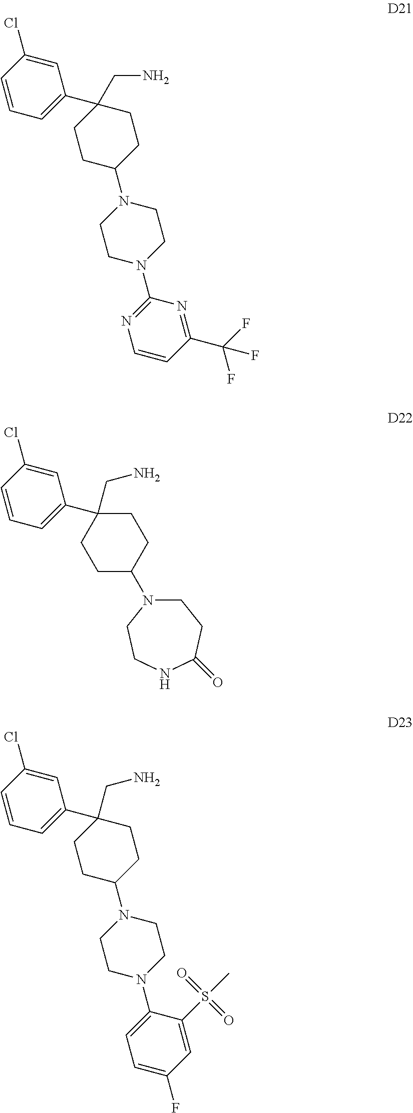 [SMILES: NCC1(c2cccc(Cl)c2)CCC(N2CCN(c3nccc(C(F)(F)F)n3)CC2)CC1 ; NCC1(c2cccc(Cl)c2)CCC(N2CCNC(=O)CC2)CC1 ; CS(=O)(=O)c1cc(F)ccc1N1CCN(C2CCC(CN)(c3cccc(Cl)c3)CC2)CC1]

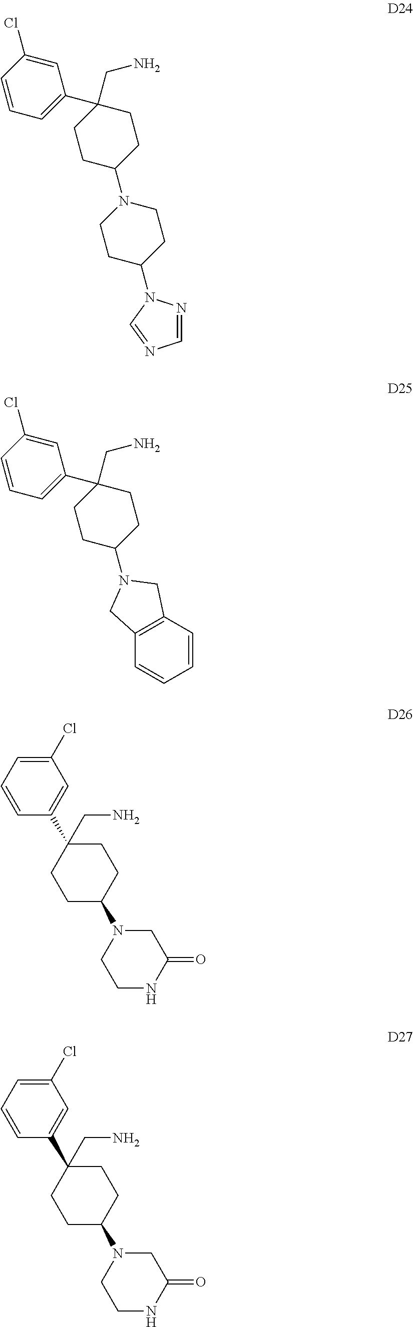 [SMILES: NCC1(c2cccc(Cl)c2)CCC(N2CCC(n3cncn3)CC2)CC1 ; NCC1(c2cccc(Cl)c2)CCC(N2Cc3ccccc3C2)CC1 ; NC[C@]1(c2cccc(Cl)c2)CC[C@H](N2CCNC(=O)C2)CC1 ; NC[C@]1(c2cccc(Cl)c2)CC[C@@H](N2CCNC(=O)C2)CC1]

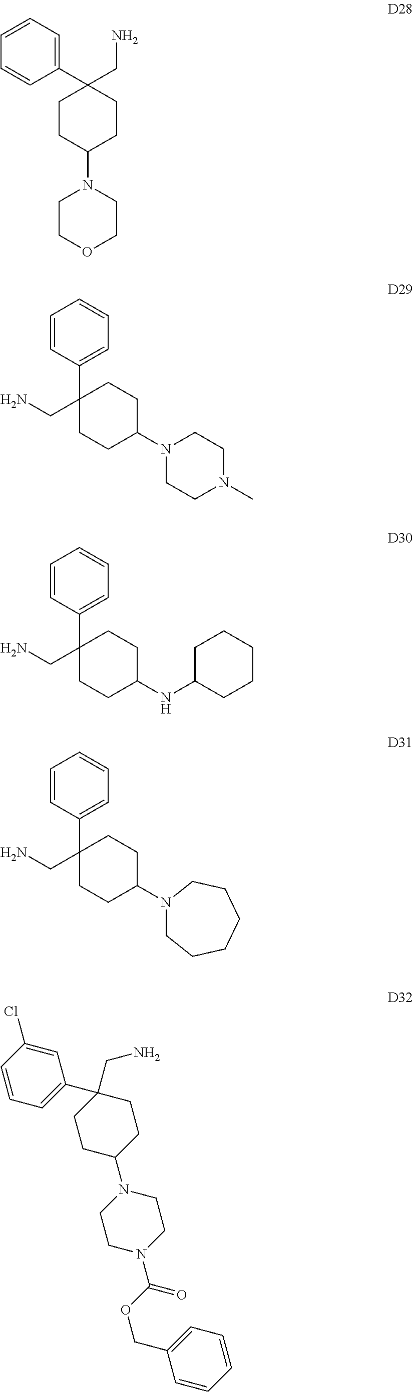 [SMILES: NCC1(c2ccccc2)CCC(N2CCOCC2)CC1 ; CN1CCN(C2CCC(CN)(c3ccccc3)CC2)CC1 ; NCC1(c2ccccc2)CCC(NC2CCCCC2)CC1 ; NCC1(c2ccccc2)CCC(N2CCCCCC2)CC1 ; NCC1(c2cccc(Cl)c2)CCC(N2CCN(C(=O)OCc3ccccc3)CC2)CC1]

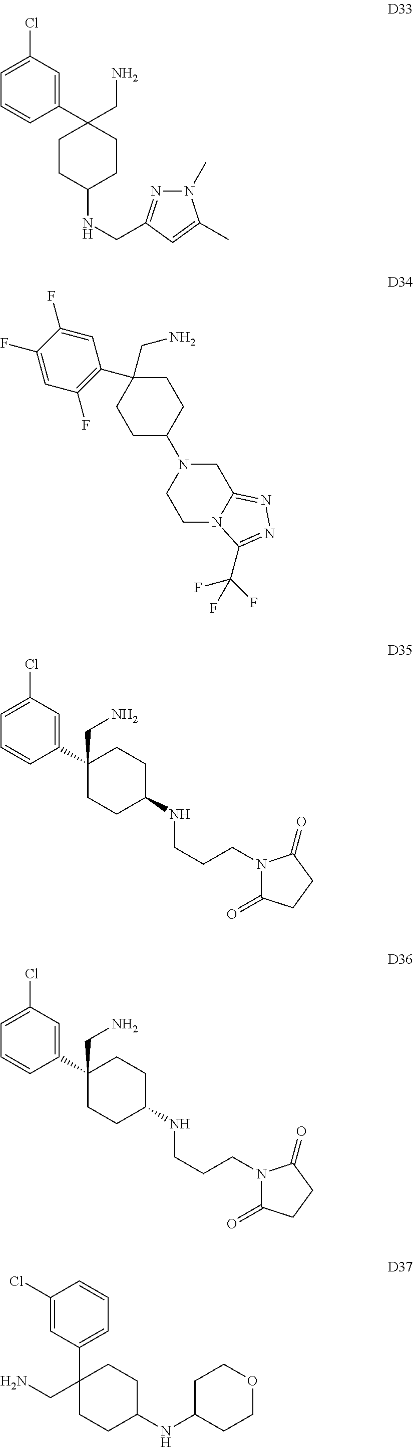 [SMILES: Cc1cc(CNC2CCC(CN)(c3cccc(Cl)c3)CC2)nn1C ; NCC1(c2cc(F)c(F)cc2F)CCC(N2CCn3c(nnc3C(F)(F)F)C2)CC1 ; NC[C@]1(c2cccc(Cl)c2)CC[C@H](NCCCN2C(=O)CCC2=O)CC1 ; NC[C@]1(c2cccc(Cl)c2)CC[C@@H](NCCCN2C(=O)CCC2=O)CC1 ; NCC1(c2cccc(Cl)c2)CCC(NC2CCOCC2)CC1]

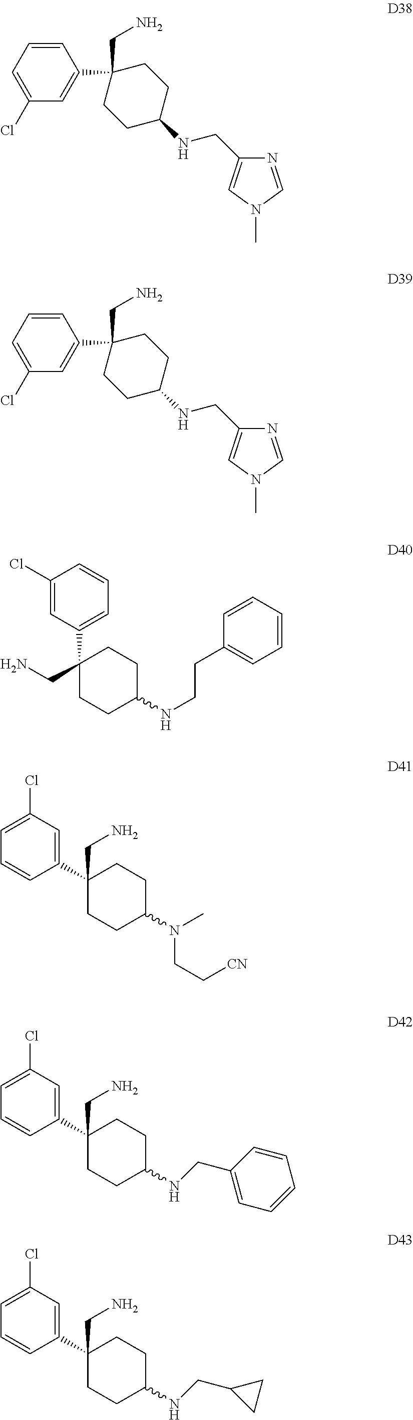

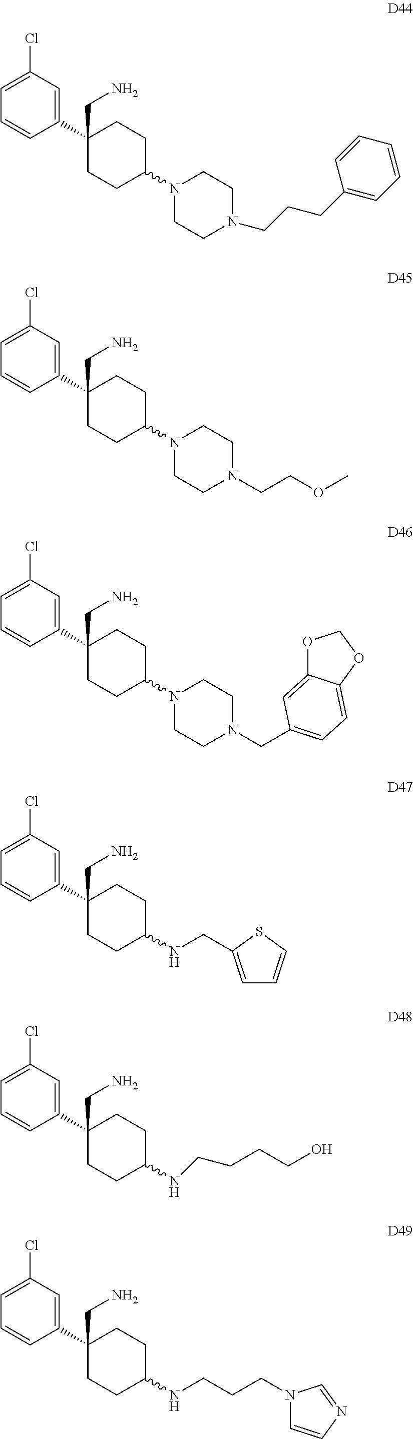

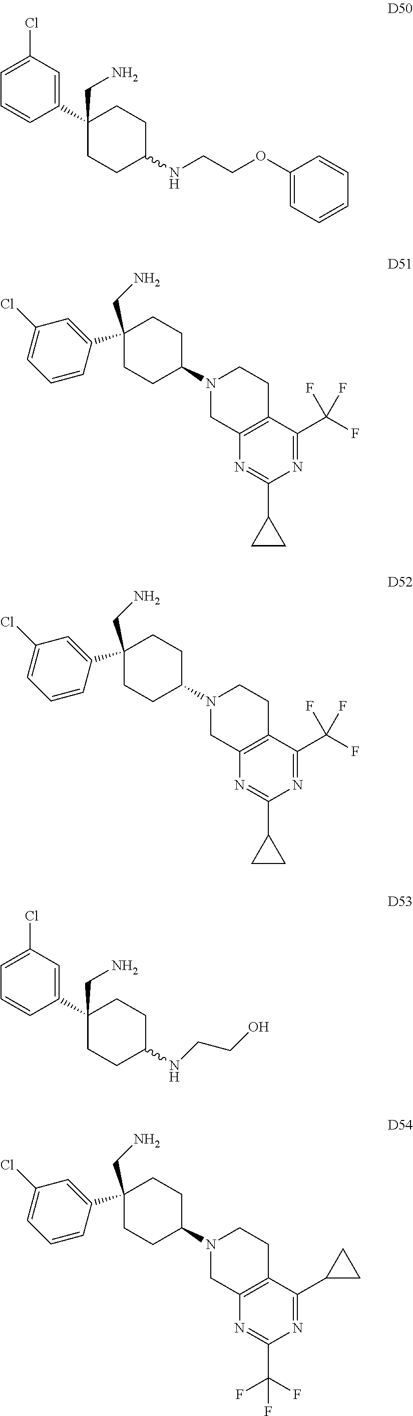

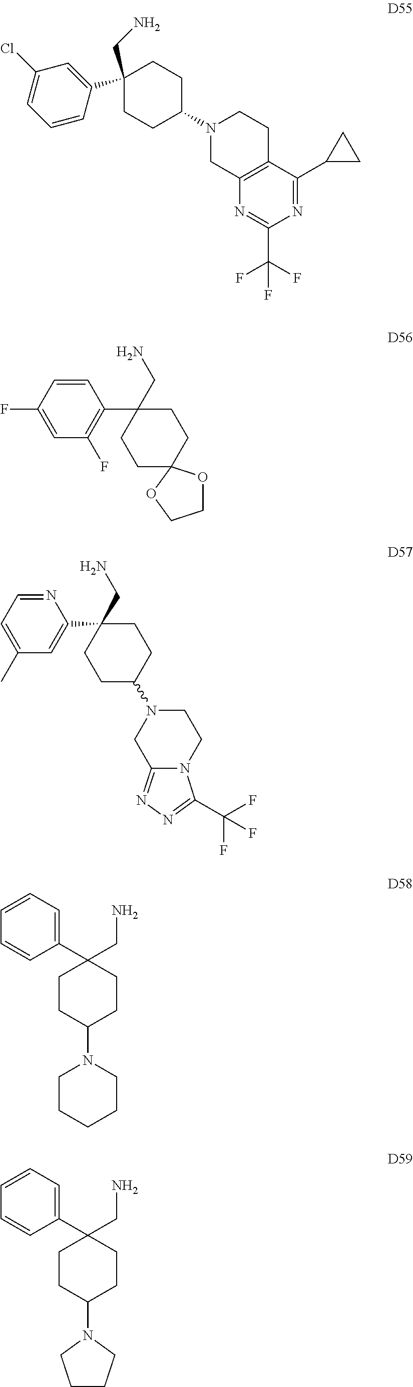

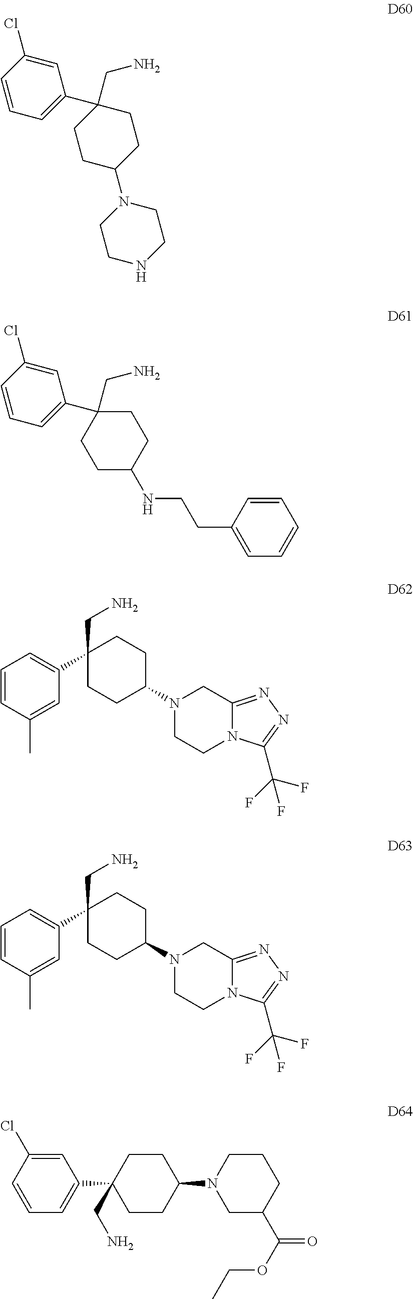 [SMILES: NCC1(c2cccc(Cl)c2)CCC(N2CCNCC2)CC1 ; NCC1(c2cccc(Cl)c2)CCC(NCCc2ccccc2)CC1 ; Cc1cccc([C@]2(CN)CC[C@H](N3CCn4c(nnc4C(F)(F)F)C3)CC2)c1 ; Cc1cccc([C@]2(CN)CC[C@@H](N3CCn4c(nnc4C(F)(F)F)C3)CC2)c1 ; CCOC(=O)C1CCCN([C@H]2CC[C@](CN)(c3cccc(Cl)c3)CC2)C1]

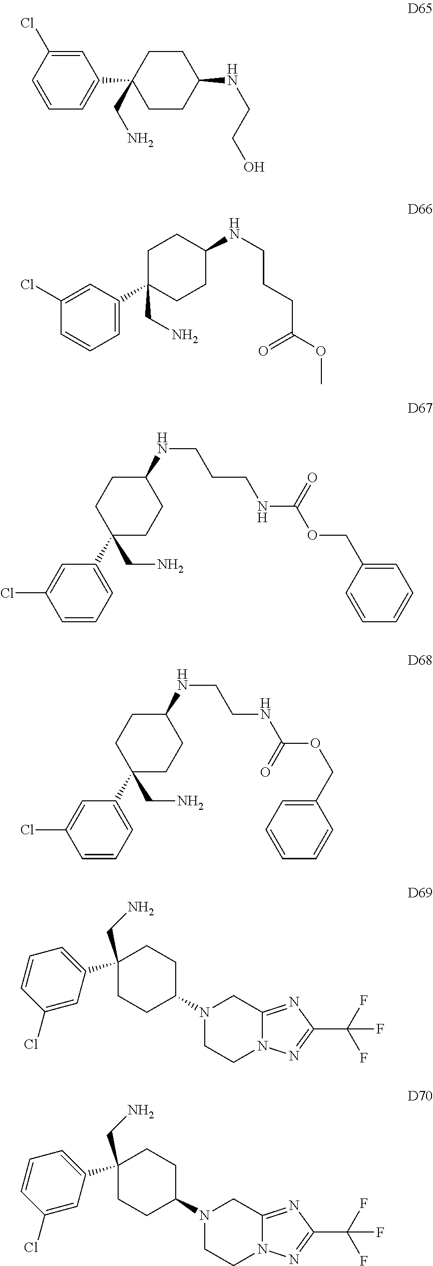 [SMILES: NC[C@]1(c2cccc(Cl)c2)CC[C@H](NCCO)CC1 ; COC(=O)CCCN[C@H]1CC[C@](CN)(c2cccc(Cl)c2)CC1 ; NC[C@]1(c2cccc(Cl)c2)CC[C@H](NCCCNC(=O)OCc2ccccc2)CC1 ; NC[C@]1(c2cccc(Cl)c2)CC[C@H](NCCNC(=O)OCc2ccccc2)CC1 ; NC[C@]1(c2cccc(Cl)c2)CC[C@@H](N2CCn3nc(C(F)(F)F)nc3C2)CC1 ; NC[C@]1(c2cccc(Cl)c2)CC[C@H](N2CCn3nc(C(F)(F)F)nc3C2)CC1]

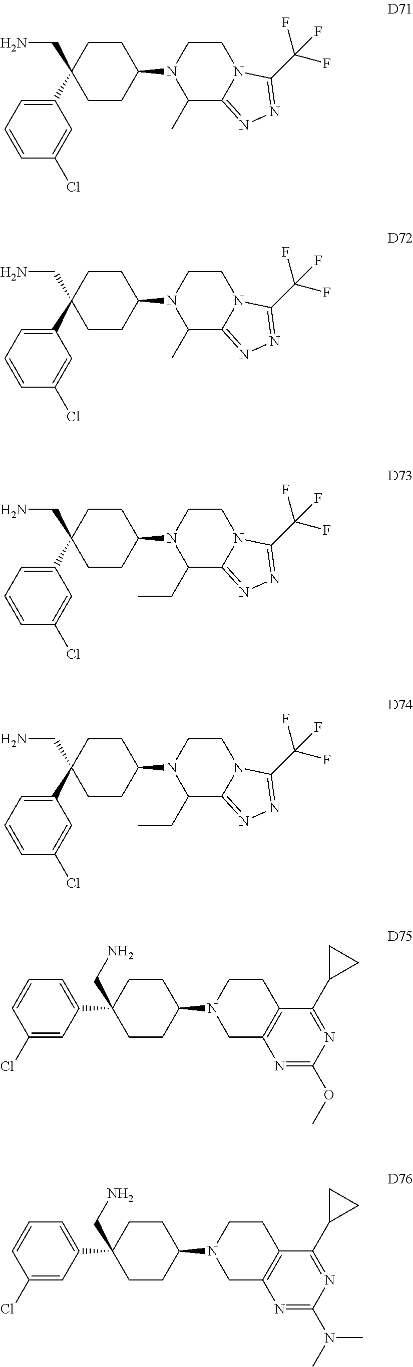 [SMILES: CC1c2nnc(C(F)(F)F)n2CCN1[C@H]1CC[C@](CN)(c2cccc(Cl)c2)CC1 ; CC1c2nnc(C(F)(F)F)n2CCN1[C@H]1CC[C@@](CN)(c2cccc(Cl)c2)CC1 ; CCC1c2nnc(C(F)(F)F)n2CCN1[C@H]1CC[C@](CN)(c2cccc(Cl)c2)CC1 ; CCC1c2nnc(C(F)(F)F)n2CCN1[C@H]1CC[C@@](CN)(c2cccc(Cl)c2)CC1 ; COc1nc2c(c(C3CC3)n1)CCN([C@H]1CC[C@](CN)(c3cccc(Cl)c3)CC1)C2 ; CN(C)c1nc2c(c(C3CC3)n1)CCN([C@H]1CC[C@](CN)(c3cccc(Cl)c3)CC1)C2]

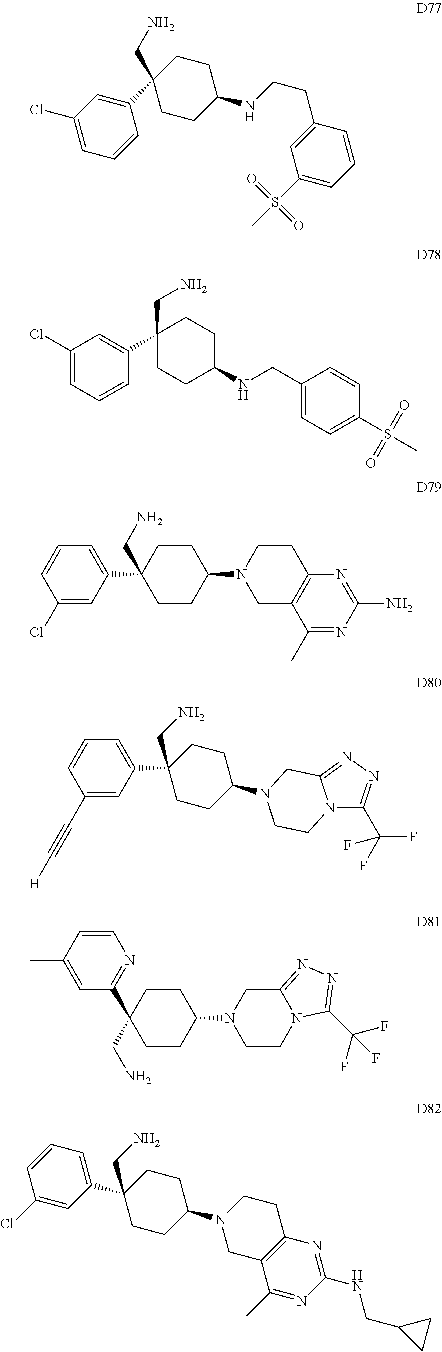

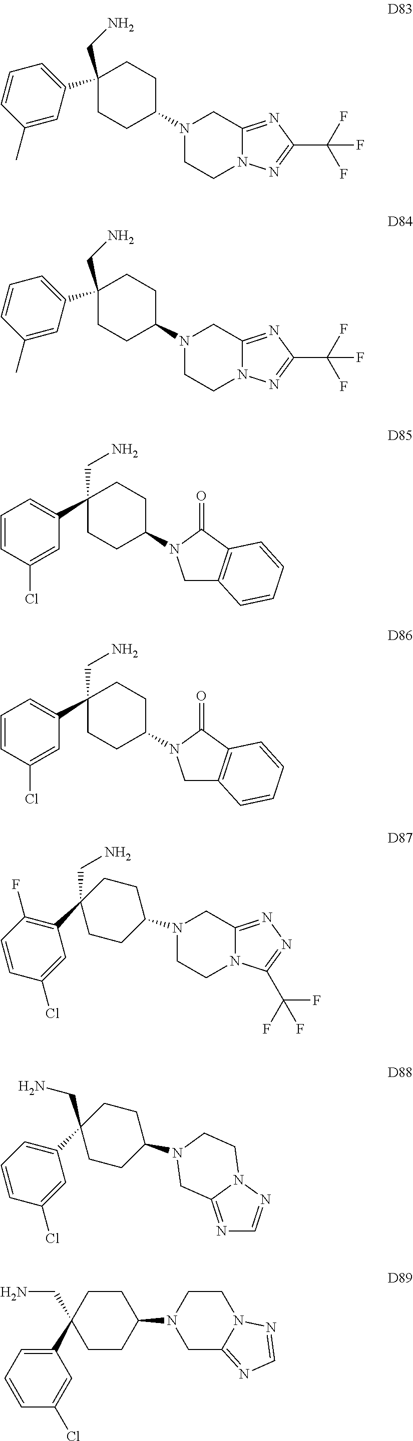

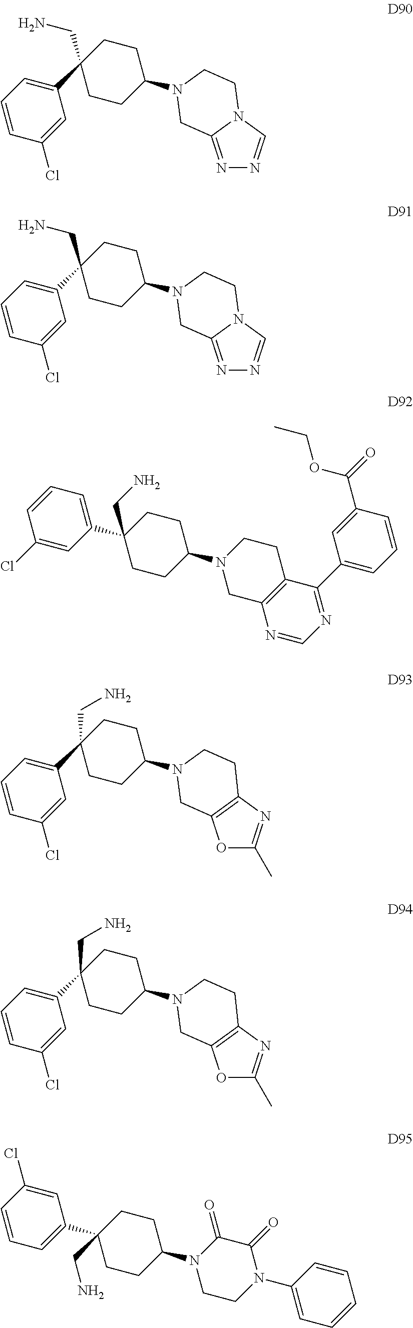 [SMILES: NC[C@]1(c2cccc(Cl)c2)CC[C@@H](N2CCn3cnnc3C2)CC1 ; NC[C@]1(c2cccc(Cl)c2)CC[C@H](N2CCn3cnnc3C2)CC1 ; CCOC(=O)c1cccc(-c2ncnc3c2CCN([C@H]2CC[C@](CN)(c4cccc(Cl)c4)CC2)C3)c1 ; Cc1nc2c(o1)CN([C@H]1CC[C@@](CN)(c3cccc(Cl)c3)CC1)CC2 ; Cc1nc2c(o1)CN([C@H]1CC[C@](CN)(c3cccc(Cl)c3)CC1)CC2 ; NC[C@]1(c2cccc(Cl)c2)CC[C@H](N2CCN(c3ccccc3)C(=O)C2=O)CC1]

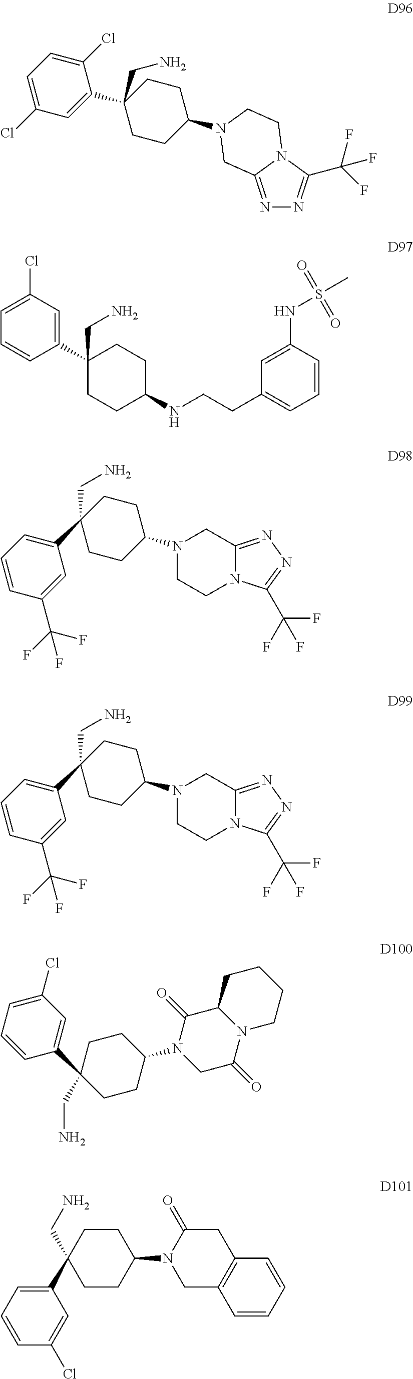 [SMILES: NC[C@]1(c2cc(Cl)ccc2Cl)CC[C@H](N2CCn3c(nnc3C(F)(F)F)C2)CC1 ; CS(=O)(=O)Nc1cccc(CCN[C@H]2CC[C@](CN)(c3cccc(Cl)c3)CC2)c1 ; NC[C@]1(c2cccc(C(F)(F)F)c2)CC[C@H](N2CCn3c(nnc3C(F)(F)F)C2)CC1 ; NC[C@]1(c2cccc(C(F)(F)F)c2)CC[C@@H](N2CCn3c(nnc3C(F)(F)F)C2)CC1 ; NC[C@]1(c2cccc(Cl)c2)CC[C@H](N2CC(=O)N3CCCC[C@@H]3C2=O)CC1 ; NC[C@]1(c2cccc(Cl)c2)CC[C@@H](N2Cc3ccccc3CC2=O)CC1]

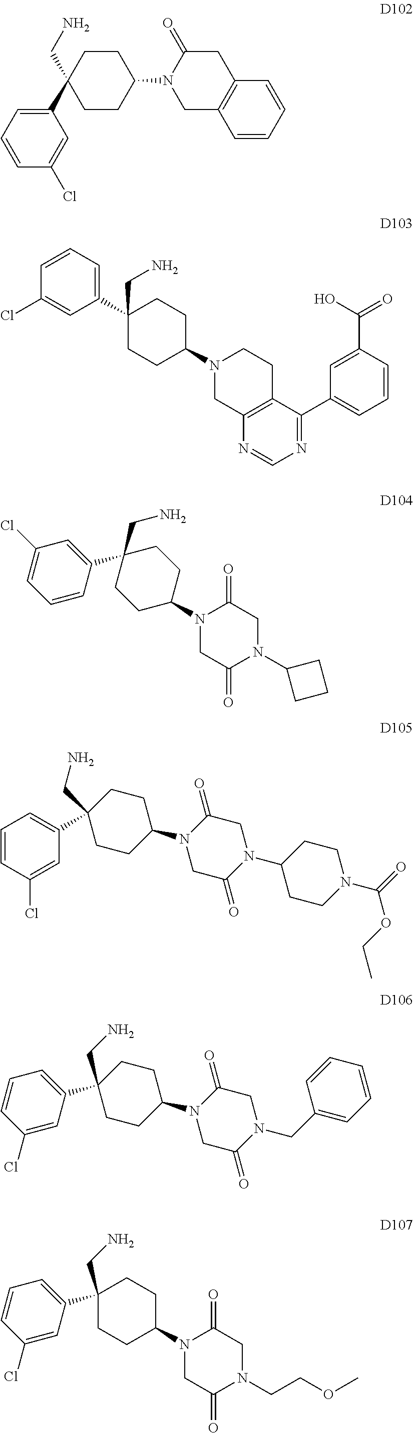 [SMILES: NC[C@]1(c2cccc(Cl)c2)CC[C@H](N2Cc3ccccc3CC2=O)CC1 ; NC[C@]1(c2cccc(Cl)c2)CC[C@H](N2CCc3c(ncnc3-c3cccc(C(=O)O)c3)C2)CC1 ; NC[C@]1(c2cccc(Cl)c2)CC[C@H](N2CC(=O)N(C3CCC3)CC2=O)CC1 ; CCOC(=O)N1CCC(N2CC(=O)N([C@H]3CC[C@](CN)(c4cccc(Cl)c4)CC3)CC2=O)CC1 ; NC[C@]1(c2cccc(Cl)c2)CC[C@H](N2CC(=O)N(Cc3ccccc3)CC2=O)CC1 ; COCCN1CC(=O)N([C@H]2CC[C@](CN)(c3cccc(Cl)c3)CC2)CC1=O]

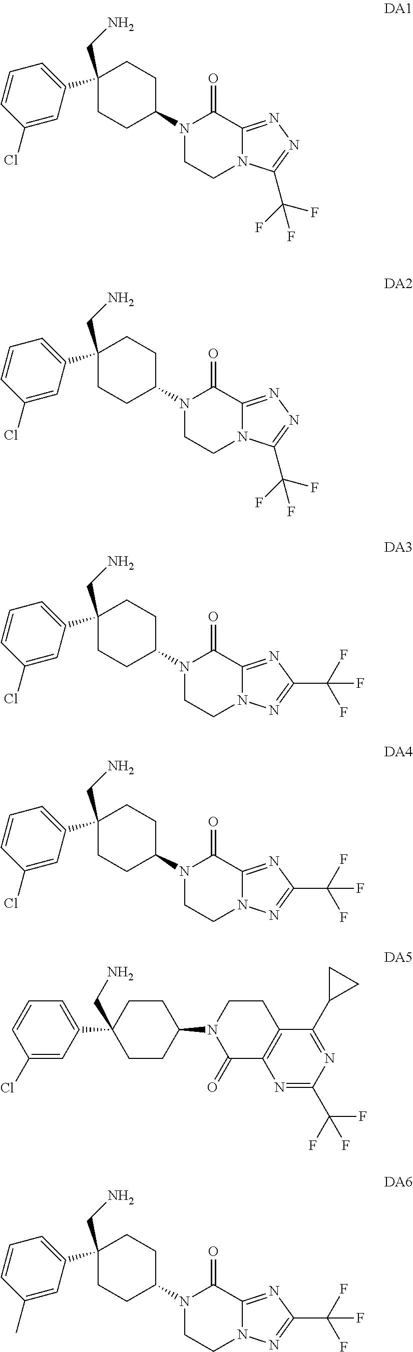 [SMILES: NC[C@]1(c2cccc(Cl)c2)CC[C@H](N2CCn3c(nnc3C(F)(F)F)C2=O)CC1 ; NC[C@]1(c2cccc(Cl)c2)CC[C@@H](N2CCn3c(nnc3C(F)(F)F)C2=O)CC1 ; NC[C@]1(c2cccc(Cl)c2)CC[C@@H](N2CCn3nc(C(F)(F)F)nc3C2=O)CC1 ; NC[C@]1(c2cccc(Cl)c2)CC[C@H](N2CCn3nc(C(F)(F)F)nc3C2=O)CC1 ; NC[C@]1(c2cccc(Cl)c2)CC[C@H](N2CCc3c(nc(C(F)(F)F)nc3C3CC3)C2=O)CC1 ; Cc1cccc([C@]2(CN)CC[C@H](N3CCn4nc(C(F)(F)F)nc4C3=O)CC2)c1]

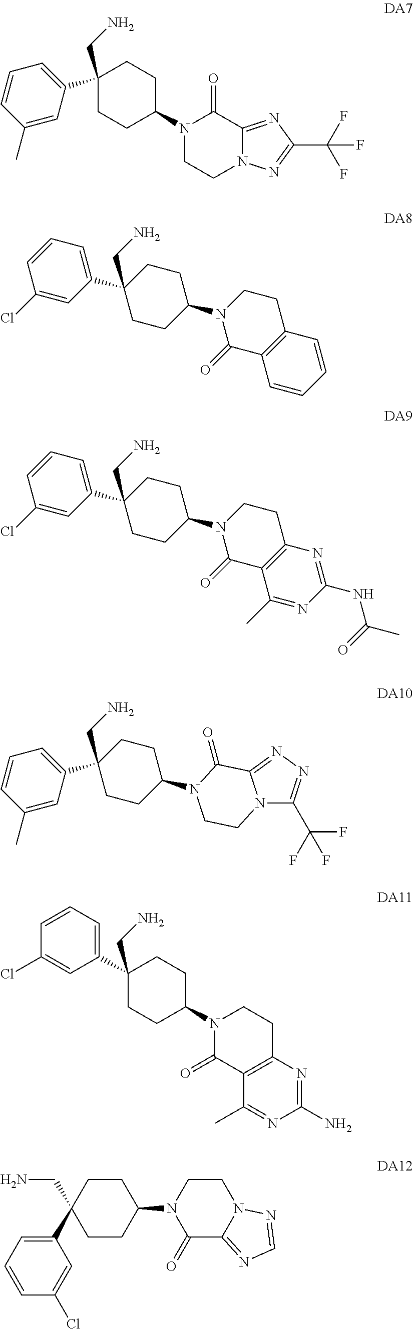 [SMILES: Cc1cccc([C@]2(CN)CC[C@@H](N3CCn4nc(C(F)(F)F)nc4C3=O)CC2)c1 ; NC[C@]1(c2cccc(Cl)c2)CC[C@H](N2CCc3ccccc3C2=O)CC1 ; CC(=O)Nc1nc(C)c2c(n1)CCN([C@H]1CC[C@](CN)(c3cccc(Cl)c3)CC1)C2=O ; Cc1cccc([C@]2(CN)CC[C@@H](N3CCn4c(nnc4C(F)(F)F)C3=O)CC2)c1 ; Cc1nc(N)nc2c1C(=O)N([C@H]1CC[C@](CN)(c3cccc(Cl)c3)CC1)CC2 ; NC[C@]1(c2cccc(Cl)c2)CC[C@@H](N2CCn3ncnc3C2=O)CC1]

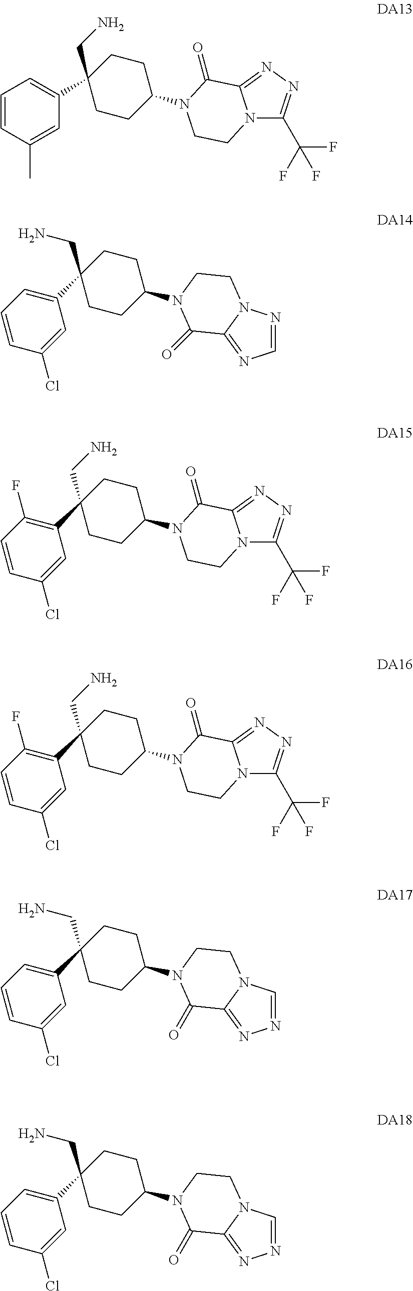 [SMILES: Cc1cccc([C@]2(CN)CC[C@H](N3CCn4c(nnc4C(F)(F)F)C3=O)CC2)c1 ; NC[C@]1(c2cccc(Cl)c2)CC[C@H](N2CCn3ncnc3C2=O)CC1 ; NC[C@]1(c2cc(Cl)ccc2F)CC[C@@H](N2CCn3c(nnc3C(F)(F)F)C2=O)CC1 ; NC[C@]1(c2cc(Cl)ccc2F)CC[C@H](N2CCn3c(nnc3C(F)(F)F)C2=O)CC1 ; NC[C@]1(c2cccc(Cl)c2)CC[C@@H](N2CCn3cnnc3C2=O)CC1 ; NC[C@]1(c2cccc(Cl)c2)CC[C@H](N2CCn3cnnc3C2=O)CC1]

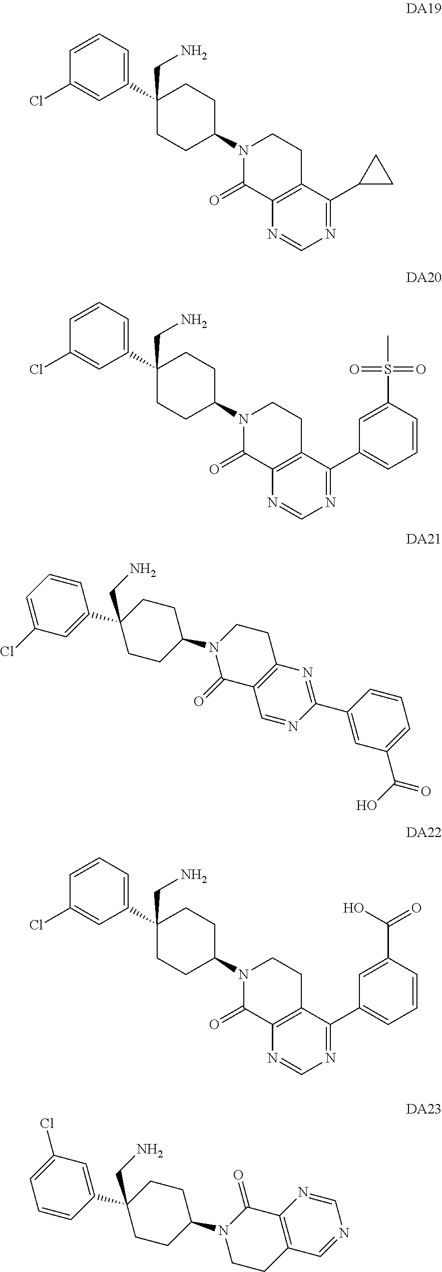 [SMILES: NC[C@]1(c2cccc(Cl)c2)CC[C@H](N2CCc3c(ncnc3C3CC3)C2=O)CC1 ; CS(=O)(=O)c1cccc(-c2ncnc3c2CCN([C@H]2CC[C@](CN)(c4cccc(Cl)c4)CC2)C3=O)c1 ; NC[C@]1(c2cccc(Cl)c2)CC[C@H](N2CCc3nc(-c4cccc(C(=O)O)c4)ncc3C2=O)CC1 ; NC[C@]1(c2cccc(Cl)c2)CC[C@H](N2CCc3c(ncnc3-c3cccc(C(=O)O)c3)C2=O)CC1 ; NC[C@]1(c2cccc(Cl)c2)CC[C@H](N2CCc3cncnc3C2=O)CC1]

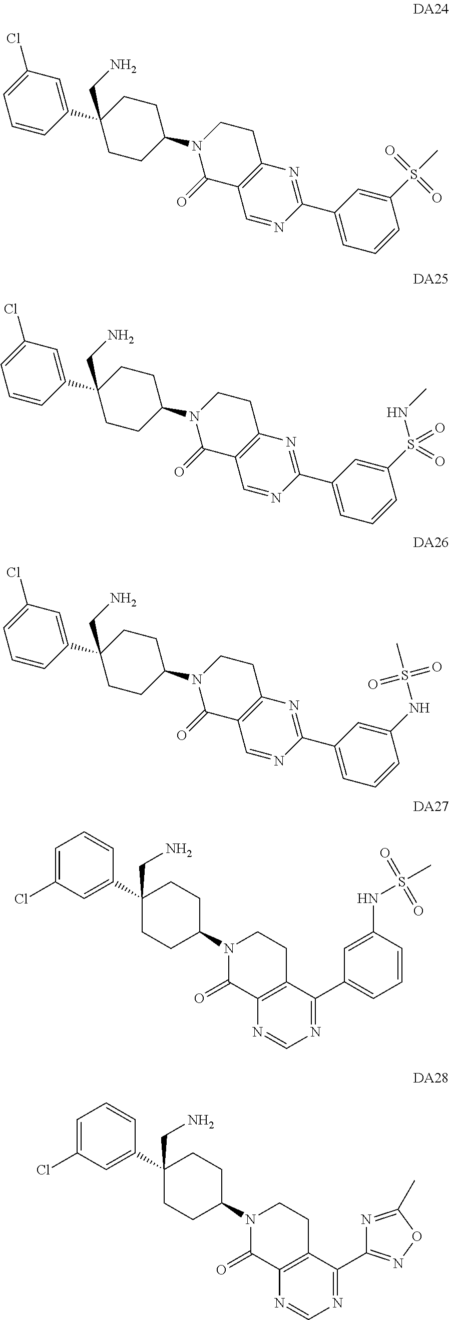 [SMILES: CS(=O)(=O)c1cccc(-c2ncc3c(n2)CCN([C@H]2CC[C@](CN)(c4cccc(Cl)c4)CC2)C3=O)c1 ; CNS(=O)(=O)c1cccc(-c2ncc3c(n2)CCN([C@H]2CC[C@](CN)(c4cccc(Cl)c4)CC2)C3=O)c1 ; CS(=O)(=O)Nc1cccc(-c2ncc3c(n2)CCN([C@H]2CC[C@](CN)(c4cccc(Cl)c4)CC2)C3=O)c1 ; CS(=O)(=O)Nc1cccc(-c2ncnc3c2CCN([C@H]2CC[C@](CN)(c4cccc(Cl)c4)CC2)C3=O)c1 ; Cc1nc(-c2ncnc3c2CCN([C@H]2CC[C@](CN)(c4cccc(Cl)c4)CC2)C3=O)no1]

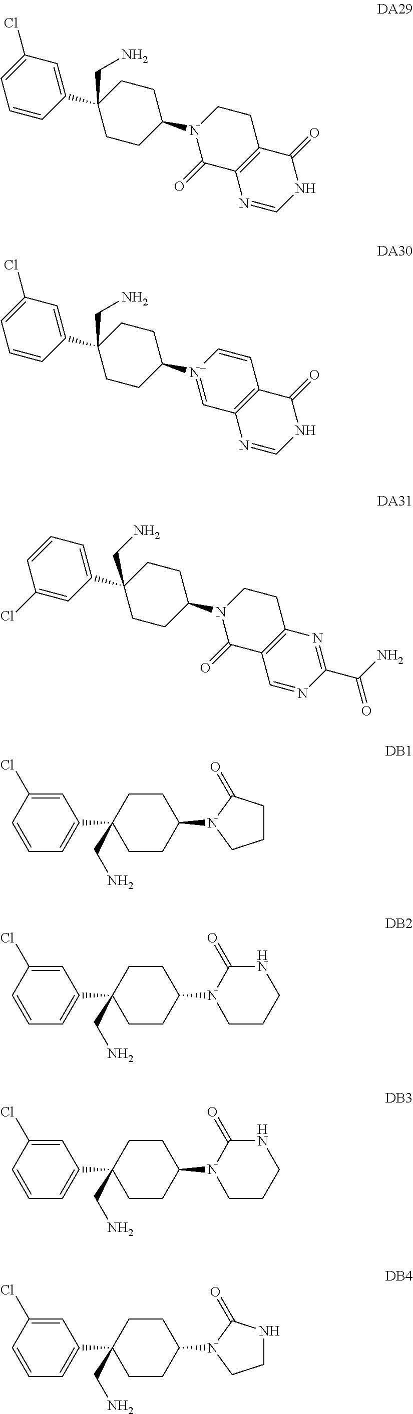

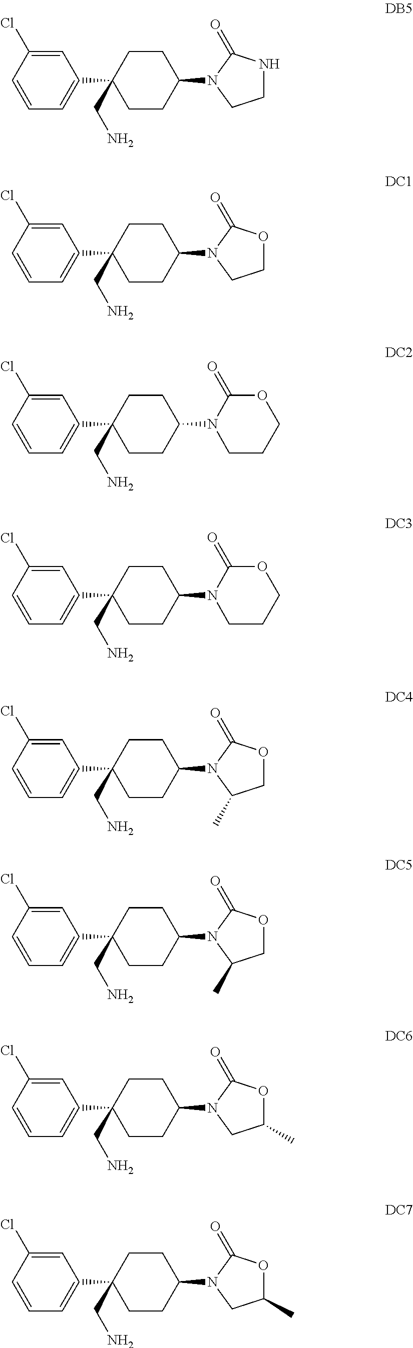

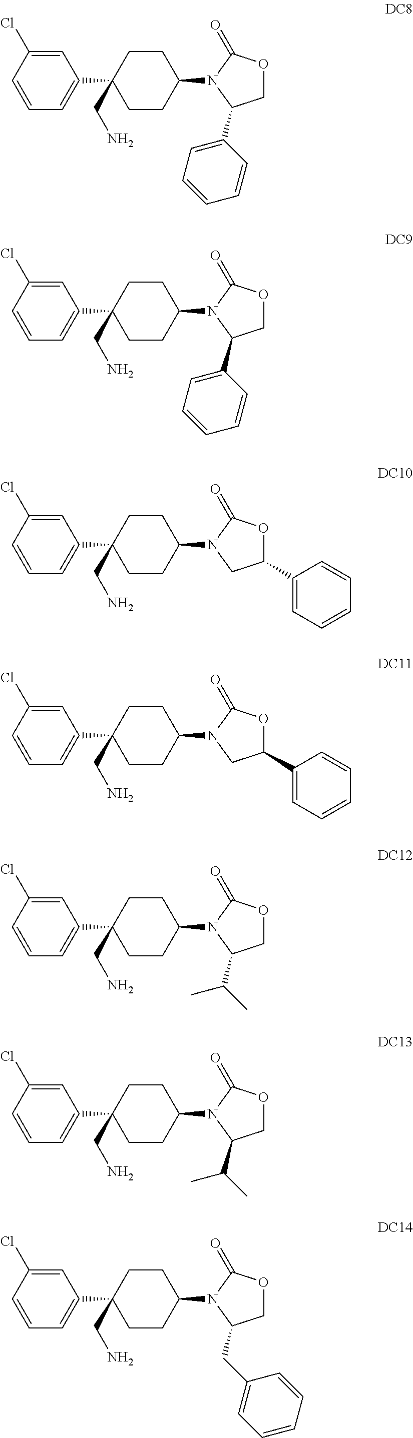

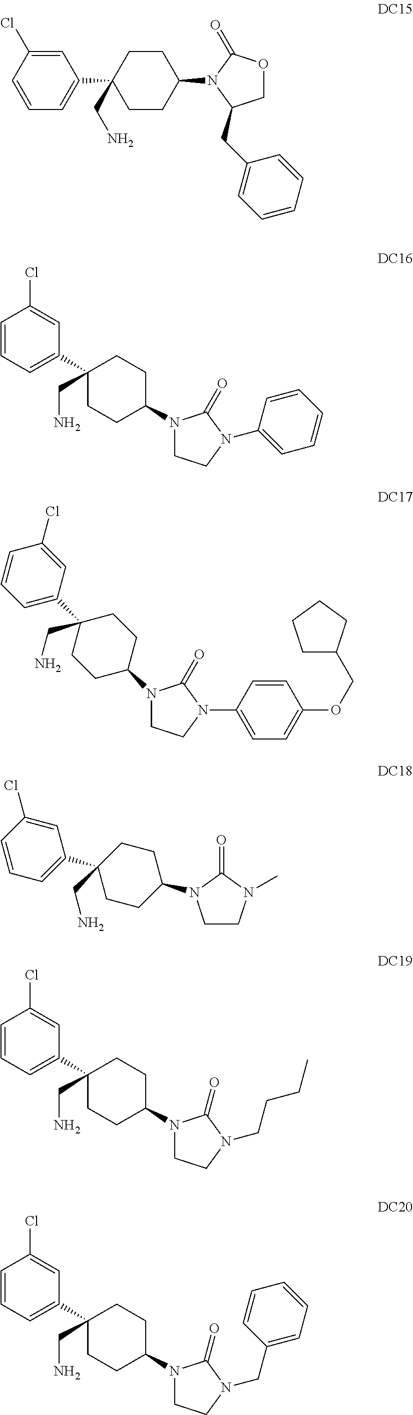 [SMILES: NC[C@]1(c2cccc(Cl)c2)CC[C@H](N2C(=O)OC[C@H]2Cc2ccccc2)CC1 ; NC[C@]1(c2cccc(Cl)c2)CC[C@H](N2CCN(c3ccccc3)C2=O)CC1 ; NC[C@]1(c2cccc(Cl)c2)CC[C@H](N2CCN(c3ccc(OCC4CCCC4)cc3)C2=O)CC1 ; CN1CCN([C@H]2CC[C@](CN)(c3cccc(Cl)c3)CC2)C1=O ; CCCCN1CCN([C@H]2CC[C@](CN)(c3cccc(Cl)c3)CC2)C1=O ; NC[C@]1(c2cccc(Cl)c2)CC[C@H](N2CCN(Cc3ccccc3)C2=O)CC1]

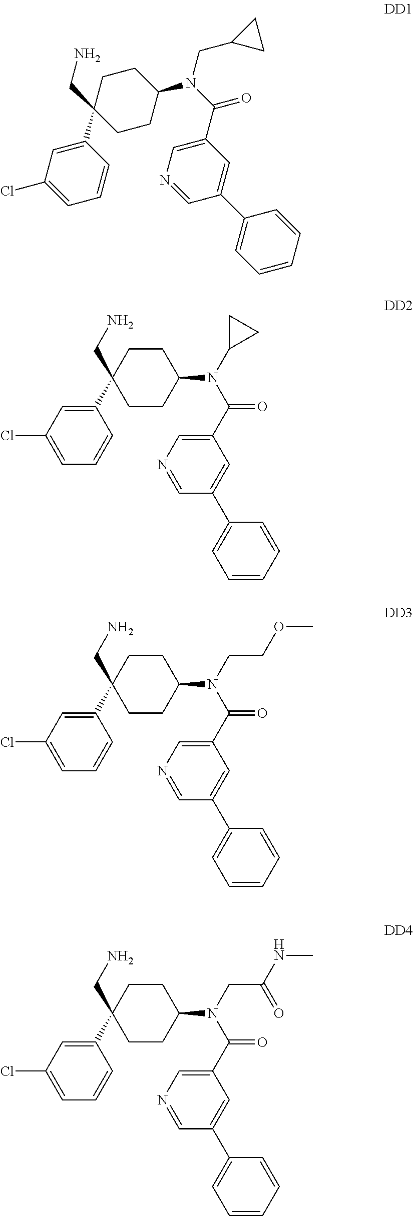 [SMILES: NC[C@]1(c2cccc(Cl)c2)CC[C@H](N(CC2CC2)C(=O)c2cncc(-c3ccccc3)c2)CC1 ; NC[C@]1(c2cccc(Cl)c2)CC[C@H](N(C(=O)c2cncc(-c3ccccc3)c2)C2CC2)CC1 ; COCCN(C(=O)c1cncc(-c2ccccc2)c1)[C@H]1CC[C@](CN)(c2cccc(Cl)c2)CC1 ; CNC(=O)CN(C(=O)c1cncc(-c2ccccc2)c1)[C@H]1CC[C@](CN)(c2cccc(Cl)c2)CC1]

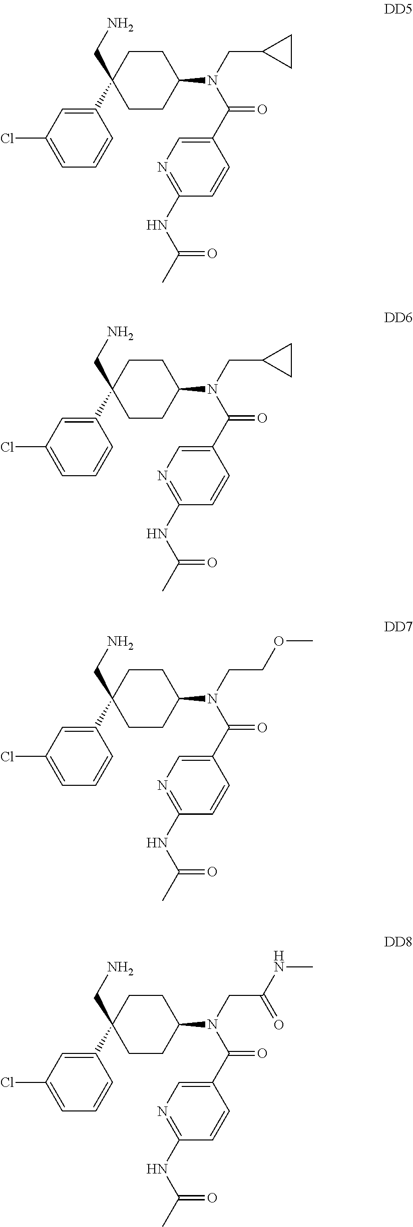 [SMILES: CC(=O)Nc1ccc(C(=O)N(CC2CC2)[C@H]2CC[C@](CN)(c3cccc(Cl)c3)CC2)cn1 ; CC(=O)Nc1ccc(C(=O)N(CC2CC2)[C@H]2CC[C@](CN)(c3cccc(Cl)c3)CC2)cn1 ; COCCN(C(=O)c1ccc(NC(C)=O)nc1)[C@H]1CC[C@](CN)(c2cccc(Cl)c2)CC1 ; CNC(=O)CN(C(=O)c1ccc(NC(C)=O)nc1)[C@H]1CC[C@](CN)(c2cccc(Cl)c2)CC1]

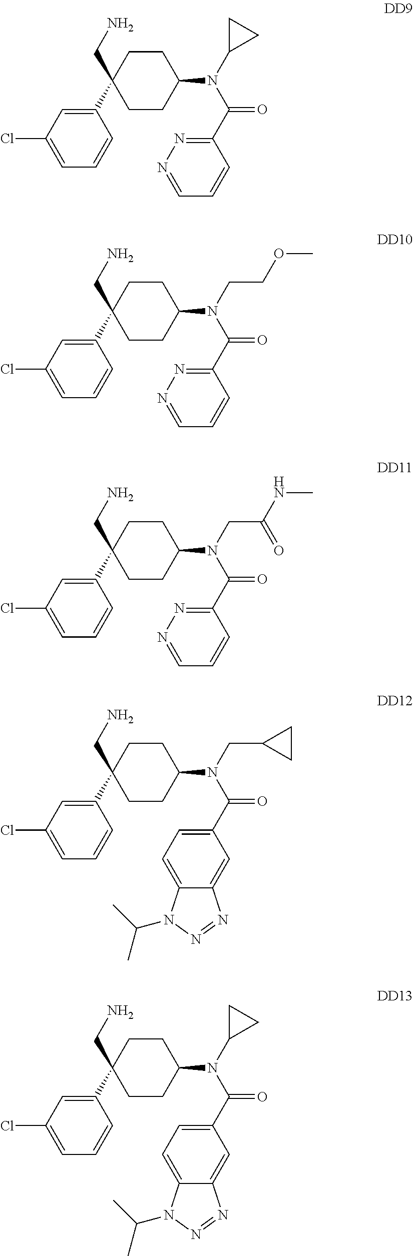 [SMILES: NC[C@]1(c2cccc(Cl)c2)CC[C@H](N(C(=O)c2cccnn2)C2CC2)CC1 ; COCCN(C(=O)c1cccnn1)[C@H]1CC[C@](CN)(c2cccc(Cl)c2)CC1 ; CNC(=O)CN(C(=O)c1cccnn1)[C@H]1CC[C@](CN)(c2cccc(Cl)c2)CC1 ; CC(C)n1nnc2cc(C(=O)N(CC3CC3)[C@H]3CC[C@](CN)(c4cccc(Cl)c4)CC3)ccc21 ; CC(C)n1nnc2cc(C(=O)N(C3CC3)[C@H]3CC[C@](CN)(c4cccc(Cl)c4)CC3)ccc21]

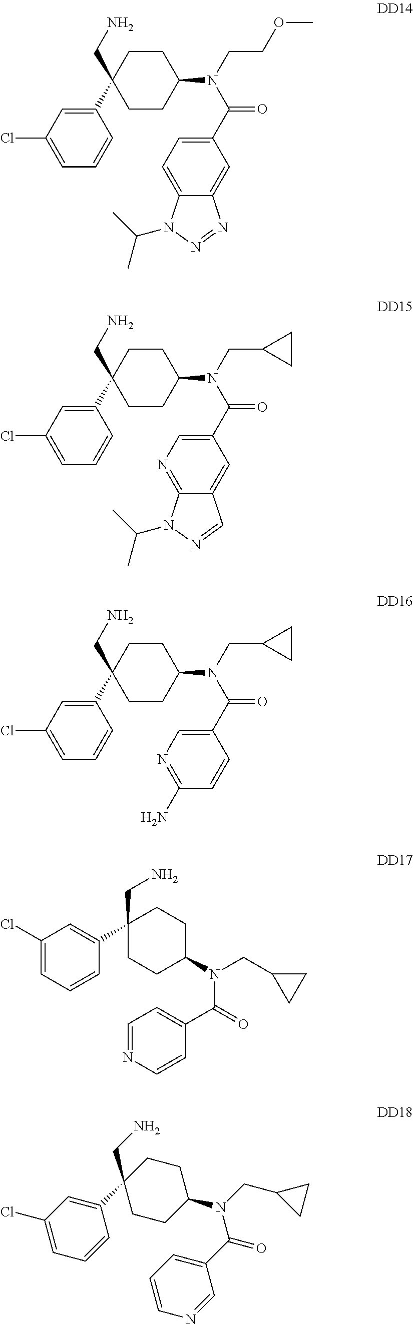 [SMILES: COCCN(C(=O)c1ccc2c(c1)nnn2C(C)C)[C@H]1CC[C@](CN)(c2cccc(Cl)c2)CC1 ; CC(C)n1ncc2cc(C(=O)N(CC3CC3)[C@H]3CC[C@](CN)(c4cccc(Cl)c4)CC3)cnc21 ; NC[C@]1(c2cccc(Cl)c2)CC[C@H](N(CC2CC2)C(=O)c2ccc(N)nc2)CC1 ; NC[C@]1(c2cccc(Cl)c2)CC[C@H](N(CC2CC2)C(=O)c2ccncc2)CC1 ; NC[C@]1(c2cccc(Cl)c2)CC[C@H](N(CC2CC2)C(=O)c2cccnc2)CC1]

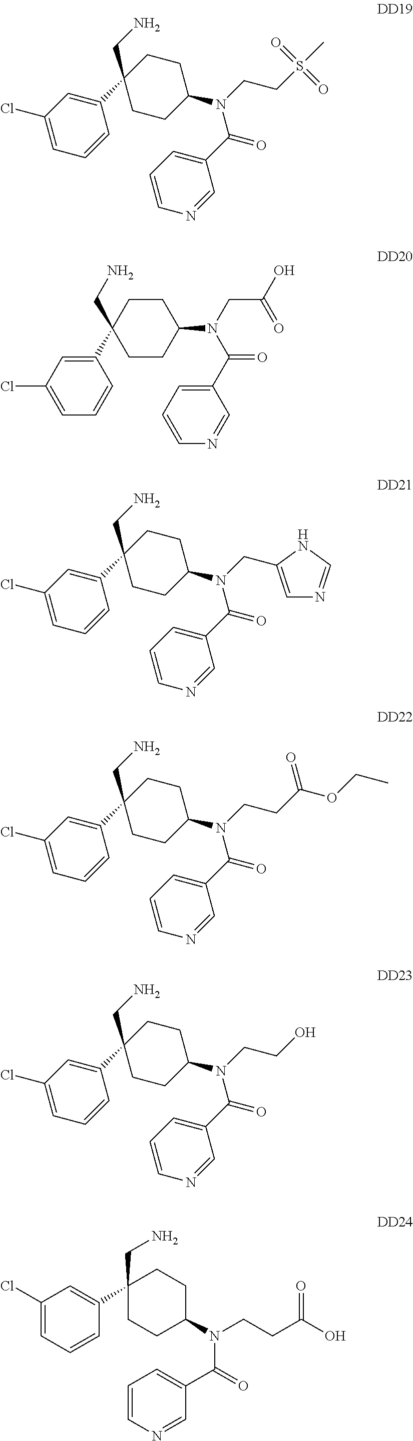 [SMILES: CS(=O)(=O)CCN(C(=O)c1cccnc1)[C@H]1CC[C@](CN)(c2cccc(Cl)c2)CC1 ; NC[C@]1(c2cccc(Cl)c2)CC[C@H](N(CC(=O)O)C(=O)c2cccnc2)CC1 ; NC[C@]1(c2cccc(Cl)c2)CC[C@H](N(Cc2cnc[nH]2)C(=O)c2cccnc2)CC1 ; CCOC(=O)CCN(C(=O)c1cccnc1)[C@H]1CC[C@](CN)(c2cccc(Cl)c2)CC1 ; NC[C@]1(c2cccc(Cl)c2)CC[C@H](N(CCO)C(=O)c2cccnc2)CC1 ; NC[C@]1(c2cccc(Cl)c2)CC[C@H](N(CCC(=O)O)C(=O)c2cccnc2)CC1]

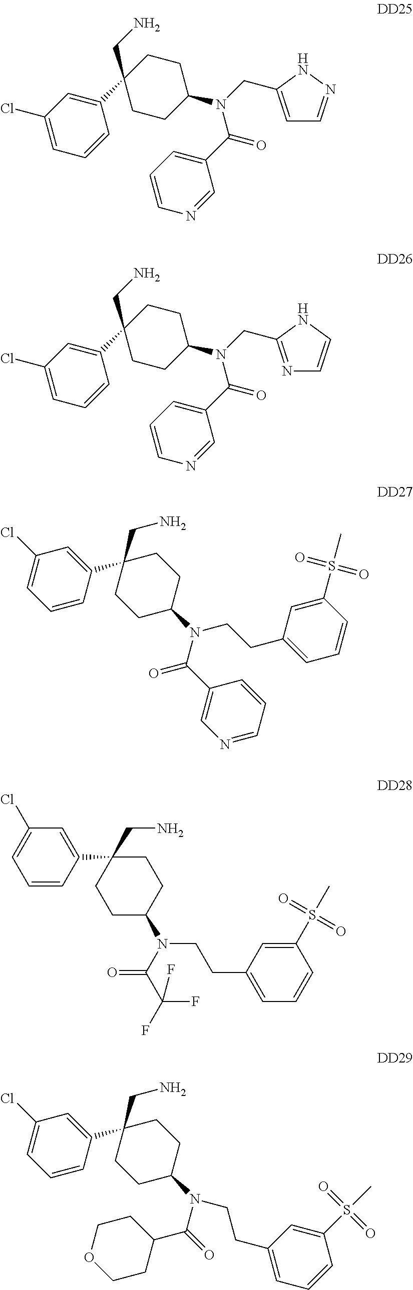 [SMILES: NC[C@]1(c2cccc(Cl)c2)CC[C@H](N(Cc2ccn[nH]2)C(=O)c2cccnc2)CC1 ; NC[C@]1(c2cccc(Cl)c2)CC[C@H](N(Cc2ncc[nH]2)C(=O)c2cccnc2)CC1 ; CS(=O)(=O)c1cccc(CCN(C(=O)c2cccnc2)[C@H]2CC[C@](CN)(c3cccc(Cl)c3)CC2)c1 ; CS(=O)(=O)c1cccc(CCN(C(=O)C(F)(F)F)[C@H]2CC[C@](CN)(c3cccc(Cl)c3)CC2)c1 ; CS(=O)(=O)c1cccc(CCN(C(=O)C2CCOCC2)[C@H]2CC[C@](CN)(c3cccc(Cl)c3)CC2)c1]

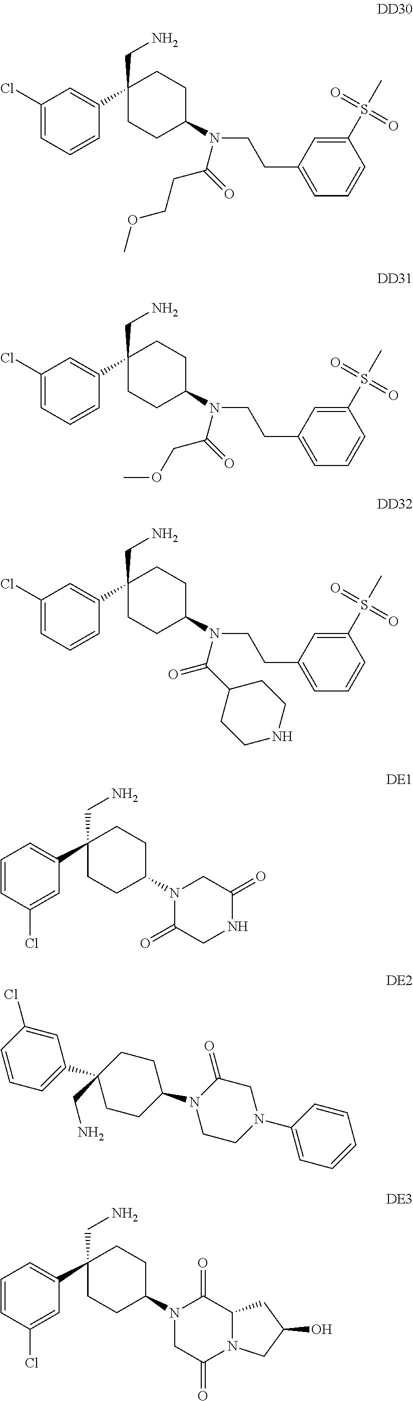 [SMILES: COCCC(=O)N(CCc1cccc(S(C)(=O)=O)c1)[C@H]1CC[C@](CN)(c2cccc(Cl)c2)CC1 ; COCC(=O)N(CCc1cccc(S(C)(=O)=O)c1)[C@H]1CC[C@](CN)(c2cccc(Cl)c2)CC1 ; CS(=O)(=O)c1cccc(CCN(C(=O)C2CCNCC2)[C@H]2CC[C@](CN)(c3cccc(Cl)c3)CC2)c1 ; NC[C@]1(c2cccc(Cl)c2)CC[C@H](N2CC(=O)NCC2=O)CC1 ; NC[C@]1(c2cccc(Cl)c2)CC[C@H](N2CCN(c3ccccc3)CC2=O)CC1 ; NC[C@]1(c2cccc(Cl)c2)CC[C@@H](N2CC(=O)N3C[C@H](O)C[C@H]3C2=O)CC1]

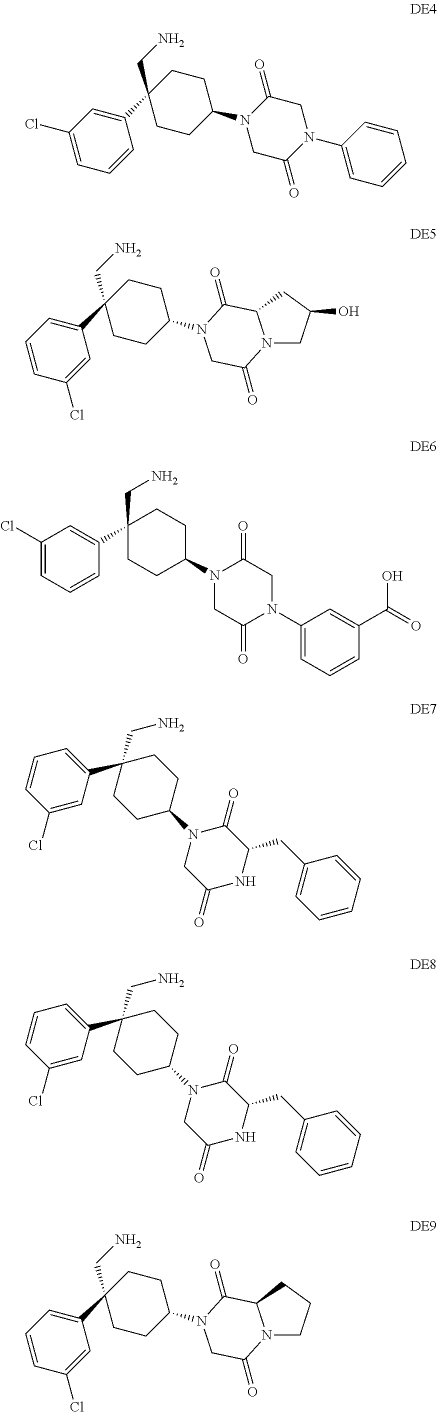 [SMILES: NC[C@]1(c2cccc(Cl)c2)CC[C@H](N2CC(=O)N(c3ccccc3)CC2=O)CC1 ; NC[C@]1(c2cccc(Cl)c2)CC[C@H](N2CC(=O)N3C[C@H](O)C[C@H]3C2=O)CC1 ; NC[C@]1(c2cccc(Cl)c2)CC[C@H](N2CC(=O)N(c3cccc(C(=O)O)c3)CC2=O)CC1 ; NC[C@]1(c2cccc(Cl)c2)CC[C@@H](N2CC(=O)N[C@@H](Cc3ccccc3)C2=O)CC1 ; NC[C@]1(c2cccc(Cl)c2)CC[C@H](N2CC(=O)N[C@@H](Cc3ccccc3)C2=O)CC1 ; NC[C@]1(c2cccc(Cl)c2)CC[C@H](N2CC(=O)N3CCC[C@@H]3C2=O)CC1]

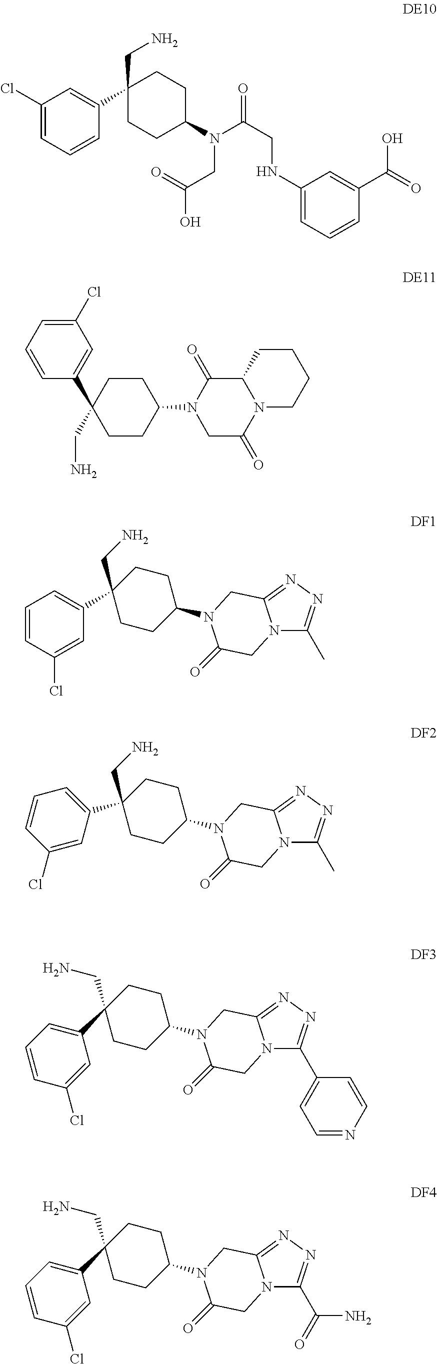 [SMILES: NC[C@]1(c2cccc(Cl)c2)CC[C@H](N(CC(=O)O)C(=O)CNc2cccc(C(=O)O)c2)CC1 ; NC[C@]1(c2cccc(Cl)c2)CC[C@H](N2CC(=O)N3CCCC[C@H]3C2=O)CC1 ; Cc1nnc2n1CC(=O)N([C@H]1CC[C@](CN)(c3cccc(Cl)c3)CC1)C2 ; Cc1nnc2n1CC(=O)N([C@H]1CC[C@@](CN)(c3cccc(Cl)c3)CC1)C2 ; NC[C@]1(c2cccc(Cl)c2)CC[C@H](N2Cc3nnc(-c4ccncc4)n3CC2=O)CC1 ; NC[C@]1(c2cccc(Cl)c2)CC[C@H](N2Cc3nnc(C(N)=O)n3CC2=O)CC1]

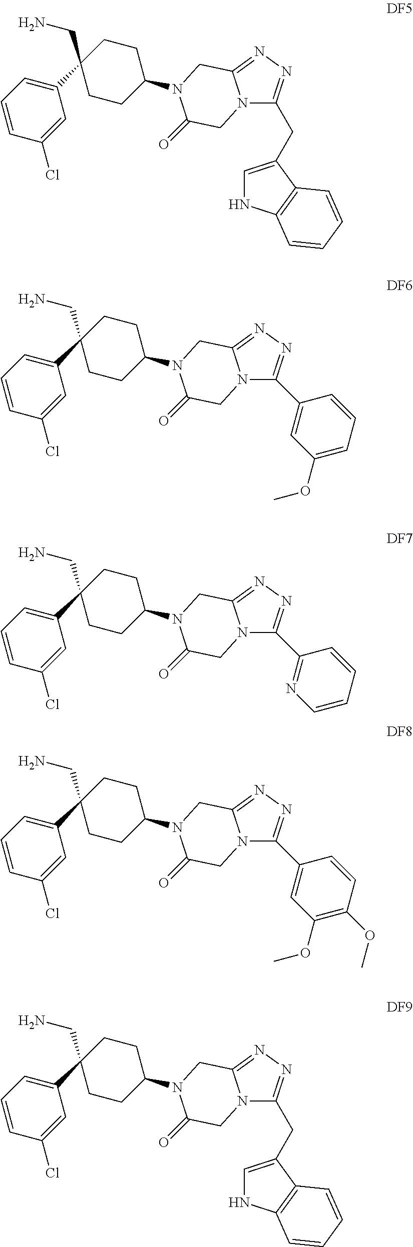 [SMILES: NC[C@]1(c2cccc(Cl)c2)CC[C@H](N2Cc3nnc(Cc4c[nH]c5ccccc45)n3CC2=O)CC1 ; COc1cccc(-c2nnc3n2CC(=O)N([C@H]2CC[C@@](CN)(c4cccc(Cl)c4)CC2)C3)c1 ; NC[C@]1(c2cccc(Cl)c2)CC[C@@H](N2Cc3nnc(-c4ccccn4)n3CC2=O)CC1 ; COc1ccc(-c2nnc3n2CC(=O)N([C@H]2CC[C@@](CN)(c4cccc(Cl)c4)CC2)C3)cc1OC ; NC[C@]1(c2cccc(Cl)c2)CC[C@@H](N2Cc3nnc(Cc4c[nH]c5ccccc45)n3CC2=O)CC1]

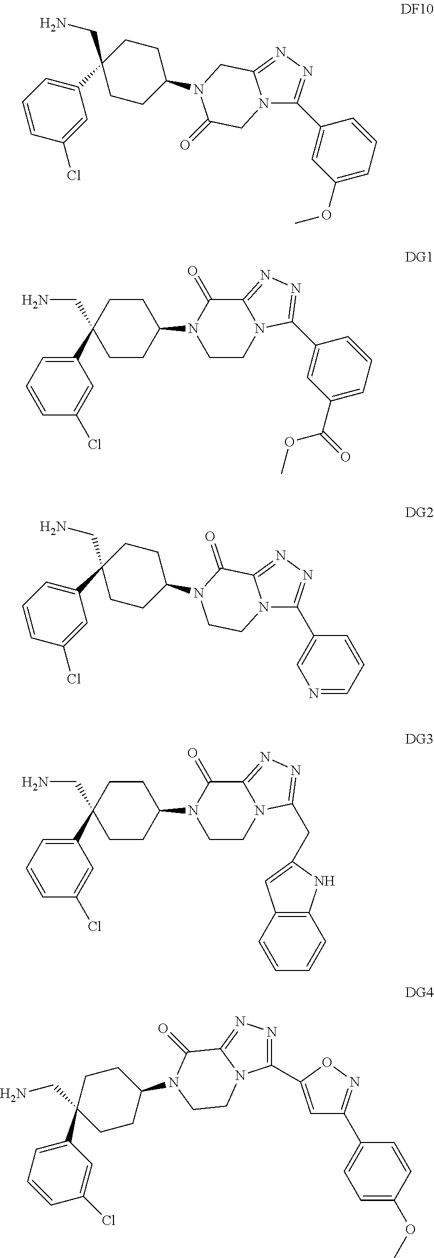 [SMILES: COc1cccc(-c2nnc3n2CC(=O)N([C@H]2CC[C@](CN)(c4cccc(Cl)c4)CC2)C3)c1 ; COC(=O)c1cccc(-c2nnc3n2CCN([C@H]2CC[C@@](CN)(c4cccc(Cl)c4)CC2)C3=O)c1 ; NC[C@]1(c2cccc(Cl)c2)CC[C@@H](N2CCn3c(nnc3-c3cccnc3)C2=O)CC1 ; NC[C@]1(c2cccc(Cl)c2)CC[C@@H](N2CCn3c(Cc4cc5ccccc5[nH]4)nnc3C2=O)CC1 ; COc1ccc(-c2cc(-c3nnc4n3CCN([C@H]3CC[C@@](CN)(c5cccc(Cl)c5)CC3)C4=O)on2)cc1]

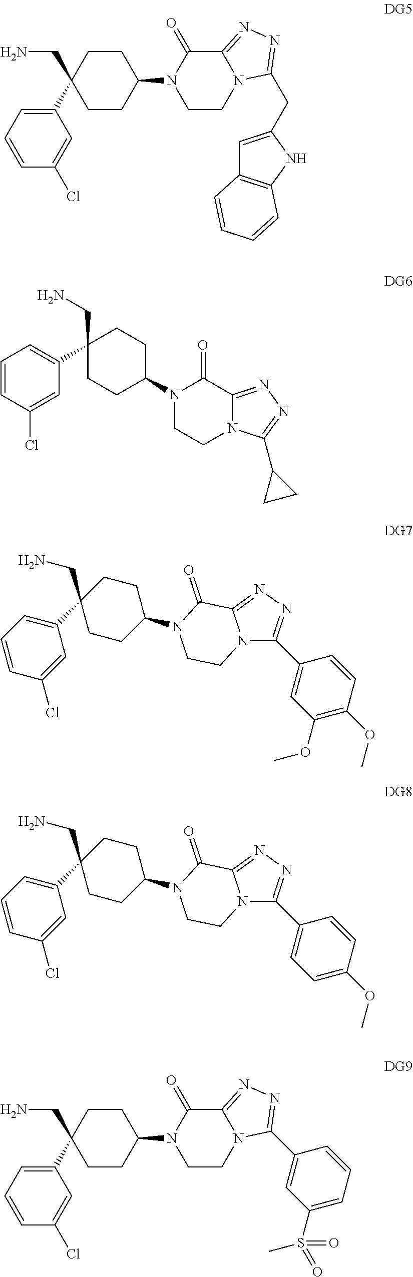 [SMILES: NC[C@]1(c2cccc(Cl)c2)CC[C@H](N2CCn3c(Cc4cc5ccccc5[nH]4)nnc3C2=O)CC1 ; NC[C@]1(c2cccc(Cl)c2)CC[C@H](N2CCn3c(nnc3C3CC3)C2=O)CC1 ; COc1ccc(-c2nnc3n2CCN([C@H]2CC[C@](CN)(c4cccc(Cl)c4)CC2)C3=O)cc1OC ; COc1ccc(-c2nnc3n2CCN([C@H]2CC[C@](CN)(c4cccc(Cl)c4)CC2)C3=O)cc1 ; CS(=O)(=O)c1cccc(-c2nnc3n2CCN([C@H]2CC[C@](CN)(c4cccc(Cl)c4)CC2)C3=O)c1]

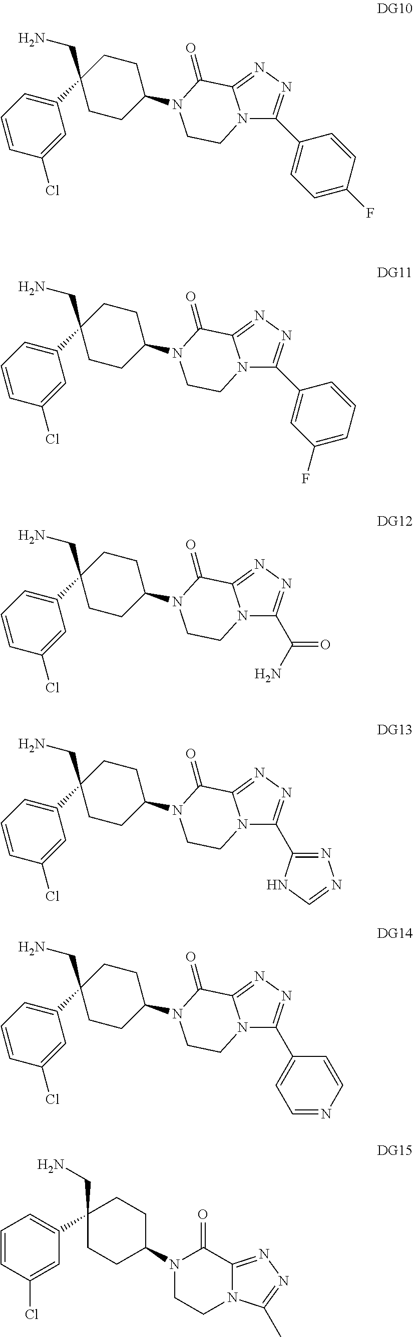 [SMILES: NC[C@]1(c2cccc(Cl)c2)CC[C@H](N2CCn3c(nnc3-c3ccc(F)cc3)C2=O)CC1 ; NC[C@]1(c2cccc(Cl)c2)CC[C@H](N2CCn3c(nnc3-c3cccc(F)c3)C2=O)CC1 ; NC[C@]1(c2cccc(Cl)c2)CC[C@H](N2CCn3c(C(N)=O)nnc3C2=O)CC1 ; NC[C@]1(c2cccc(Cl)c2)CC[C@H](N2CCn3c(nnc3-c3nnc[nH]3)C2=O)CC1 ; NC[C@]1(c2cccc(Cl)c2)CC[C@H](N2CCn3c(nnc3-c3ccncc3)C2=O)CC1 ; Cc1nnc2n1CCN([C@H]1CC[C@](CN)(c3cccc(Cl)c3)CC1)C2=O]

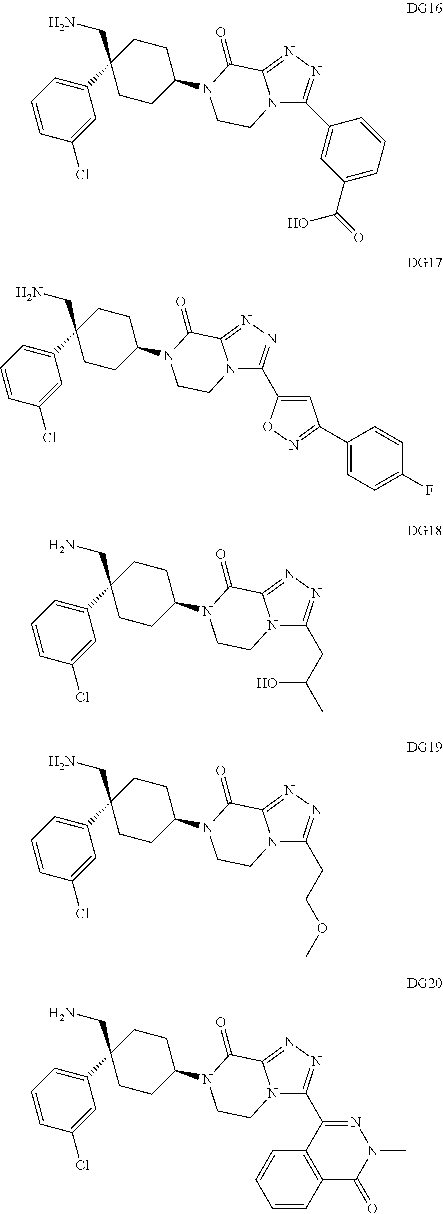 [SMILES: NC[C@]1(c2cccc(Cl)c2)CC[C@H](N2CCn3c(nnc3-c3cccc(C(=O)O)c3)C2=O)CC1 ; NC[C@]1(c2cccc(Cl)c2)CC[C@H](N2CCn3c(nnc3-c3cc(-c4ccc(F)cc4)no3)C2=O)CC1 ; CC(O)Cc1nnc2n1CCN([C@H]1CC[C@](CN)(c3cccc(Cl)c3)CC1)C2=O ; COCCc1nnc2n1CCN([C@H]1CC[C@](CN)(c3cccc(Cl)c3)CC1)C2=O ; Cn1nc(-c2nnc3n2CCN([C@H]2CC[C@](CN)(c4cccc(Cl)c4)CC2)C3=O)c2ccccc2c1=O]

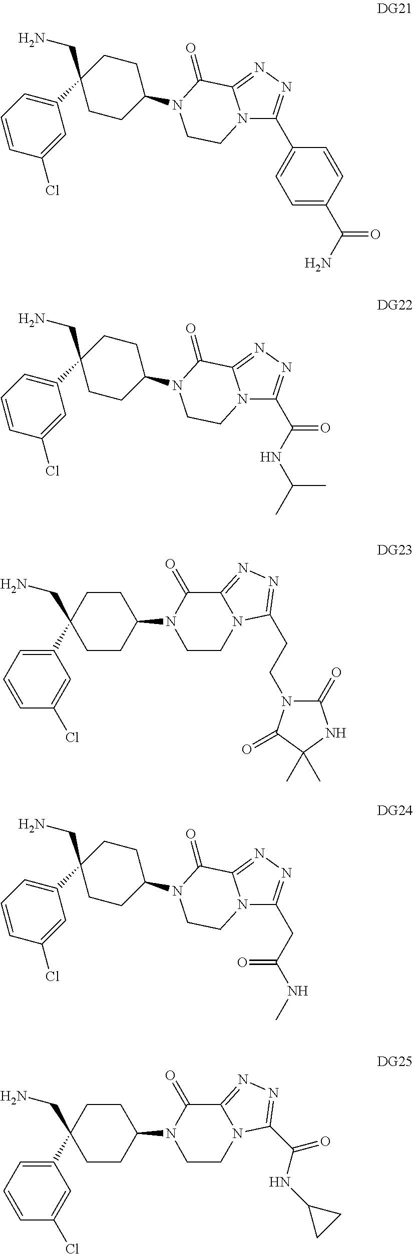 [SMILES: NC[C@]1(c2cccc(Cl)c2)CC[C@H](N2CCn3c(nnc3-c3ccc(C(N)=O)cc3)C2=O)CC1 ; CC(C)NC(=O)c1nnc2n1CCN([C@H]1CC[C@](CN)(c3cccc(Cl)c3)CC1)C2=O ; CC1(C)NC(=O)N(CCc2nnc3n2CCN([C@H]2CC[C@](CN)(c4cccc(Cl)c4)CC2)C3=O)C1=O ; CNC(=O)Cc1nnc2n1CCN([C@H]1CC[C@](CN)(c3cccc(Cl)c3)CC1)C2=O ; NC[C@]1(c2cccc(Cl)c2)CC[C@H](N2CCn3c(C(=O)NC4CC4)nnc3C2=O)CC1]

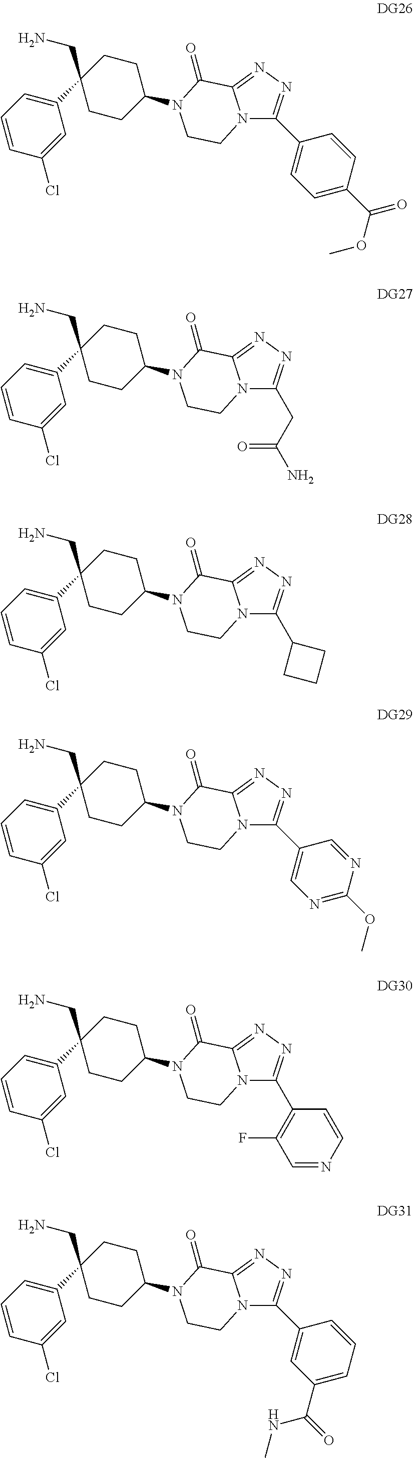 [SMILES: COC(=O)c1ccc(-c2nnc3n2CCN([C@H]2CC[C@](CN)(c4cccc(Cl)c4)CC2)C3=O)cc1 ; NC[C@]1(c2cccc(Cl)c2)CC[C@H](N2CCn3c(CC(N)=O)nnc3C2=O)CC1 ; NC[C@]1(c2cccc(Cl)c2)CC[C@H](N2CCn3c(nnc3C3CCC3)C2=O)CC1 ; COc1ncc(-c2nnc3n2CCN([C@H]2CC[C@](CN)(c4cccc(Cl)c4)CC2)C3=O)cn1 ; NC[C@]1(c2cccc(Cl)c2)CC[C@H](N2CCn3c(nnc3-c3ccncc3F)C2=O)CC1 ; CNC(=O)c1cccc(-c2nnc3n2CCN([C@H]2CC[C@](CN)(c4cccc(Cl)c4)CC2)C3=O)c1]

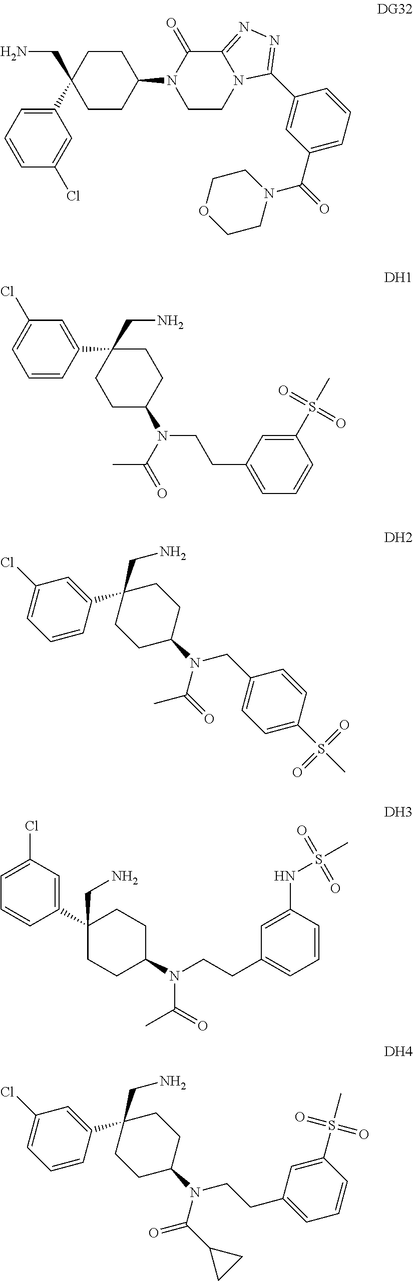 [SMILES: NC[C@]1(c2cccc(Cl)c2)CC[C@H](N2CCn3c(nnc3-c3cccc(C(=O)N4CCOCC4)c3)C2=O)CC1 ; CC(=O)N(CCc1cccc(S(C)(=O)=O)c1)[C@H]1CC[C@](CN)(c2cccc(Cl)c2)CC1 ; CC(=O)N(Cc1ccc(S(C)(=O)=O)cc1)[C@H]1CC[C@](CN)(c2cccc(Cl)c2)CC1 ; CC(=O)N(CCc1cccc(NS(C)(=O)=O)c1)[C@H]1CC[C@](CN)(c2cccc(Cl)c2)CC1 ; CS(=O)(=O)c1cccc(CCN(C(=O)C2CC2)[C@H]2CC[C@](CN)(c3cccc(Cl)c3)CC2)c1]

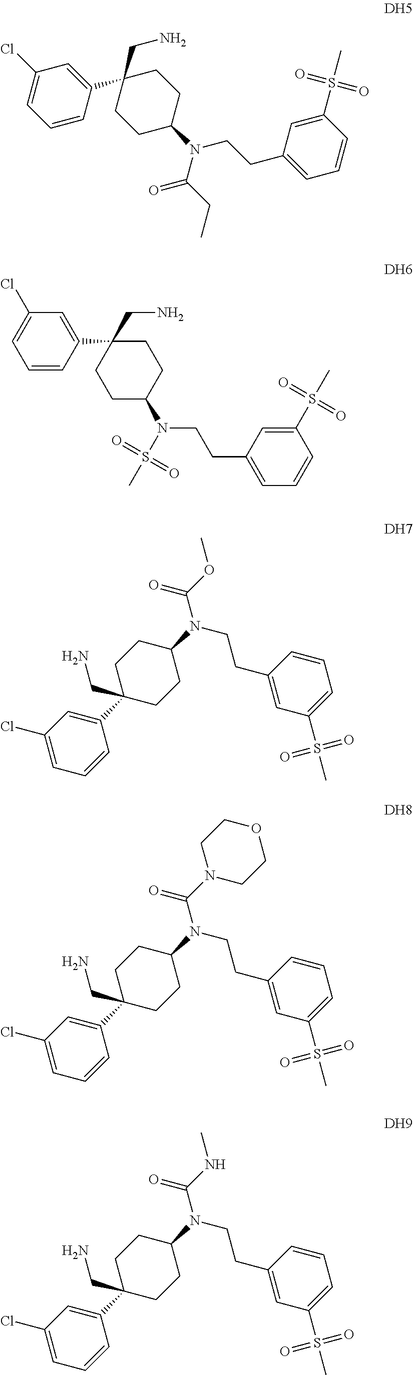 [SMILES: CCC(=O)N(CCc1cccc(S(C)(=O)=O)c1)[C@H]1CC[C@](CN)(c2cccc(Cl)c2)CC1 ; CS(=O)(=O)c1cccc(CCN([C@H]2CC[C@](CN)(c3cccc(Cl)c3)CC2)S(C)(=O)=O)c1 ; COC(=O)N(CCc1cccc(S(C)(=O)=O)c1)[C@H]1CC[C@](CN)(c2cccc(Cl)c2)CC1 ; CS(=O)(=O)c1cccc(CCN(C(=O)N2CCOCC2)[C@H]2CC[C@](CN)(c3cccc(Cl)c3)CC2)c1 ; CNC(=O)N(CCc1cccc(S(C)(=O)=O)c1)[C@H]1CC[C@](CN)(c2cccc(Cl)c2)CC1]

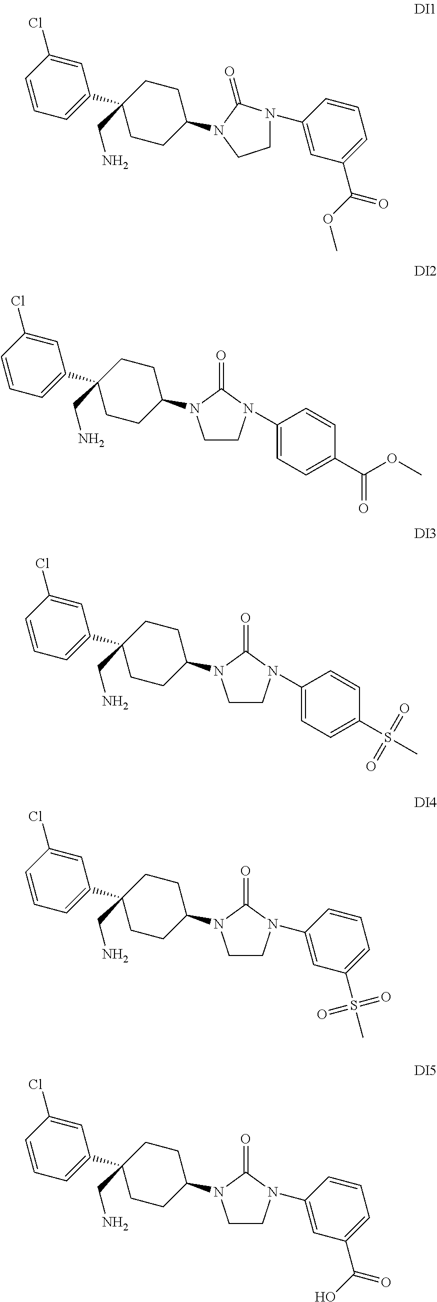 [SMILES: COC(=O)c1cccc(N2CCN([C@H]3CC[C@](CN)(c4cccc(Cl)c4)CC3)C2=O)c1 ; COC(=O)c1ccc(N2CCN([C@H]3CC[C@](CN)(c4cccc(Cl)c4)CC3)C2=O)cc1 ; CS(=O)(=O)c1ccc(N2CCN([C@H]3CC[C@](CN)(c4cccc(Cl)c4)CC3)C2=O)cc1 ; CS(=O)(=O)c1cccc(N2CCN([C@H]3CC[C@](CN)(c4cccc(Cl)c4)CC3)C2=O)c1 ; NC[C@]1(c2cccc(Cl)c2)CC[C@H](N2CCN(c3cccc(C(=O)O)c3)C2=O)CC1]

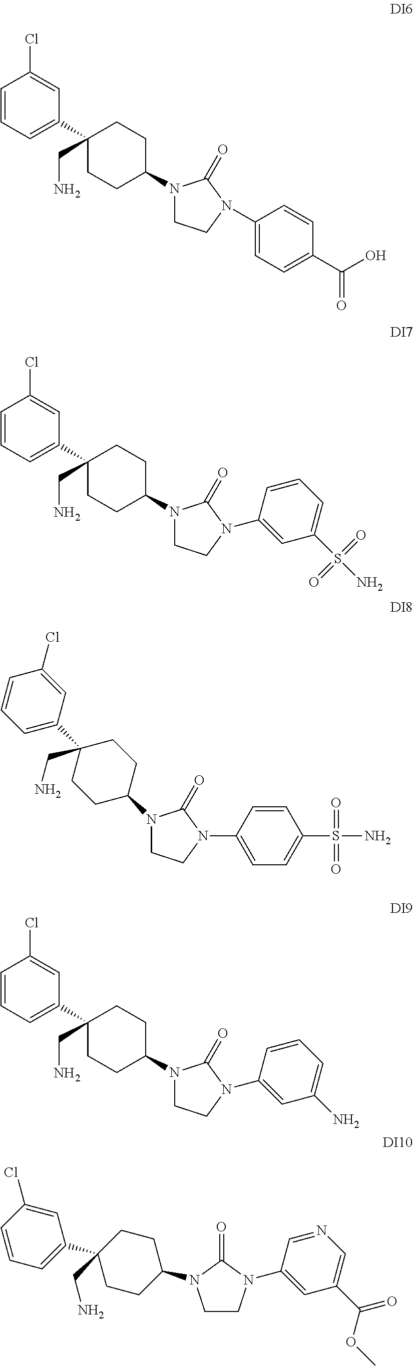 [SMILES: NC[C@]1(c2cccc(Cl)c2)CC[C@H](N2CCN(c3ccc(C(=O)O)cc3)C2=O)CC1 ; NC[C@]1(c2cccc(Cl)c2)CC[C@H](N2CCN(c3cccc(S(N)(=O)=O)c3)C2=O)CC1 ; NC[C@]1(c2cccc(Cl)c2)CC[C@H](N2CCN(c3ccc(S(N)(=O)=O)cc3)C2=O)CC1 ; NC[C@]1(c2cccc(Cl)c2)CC[C@H](N2CCN(c3cccc(N)c3)C2=O)CC1 ; COC(=O)c1cncc(N2CCN([C@H]3CC[C@](CN)(c4cccc(Cl)c4)CC3)C2=O)c1]

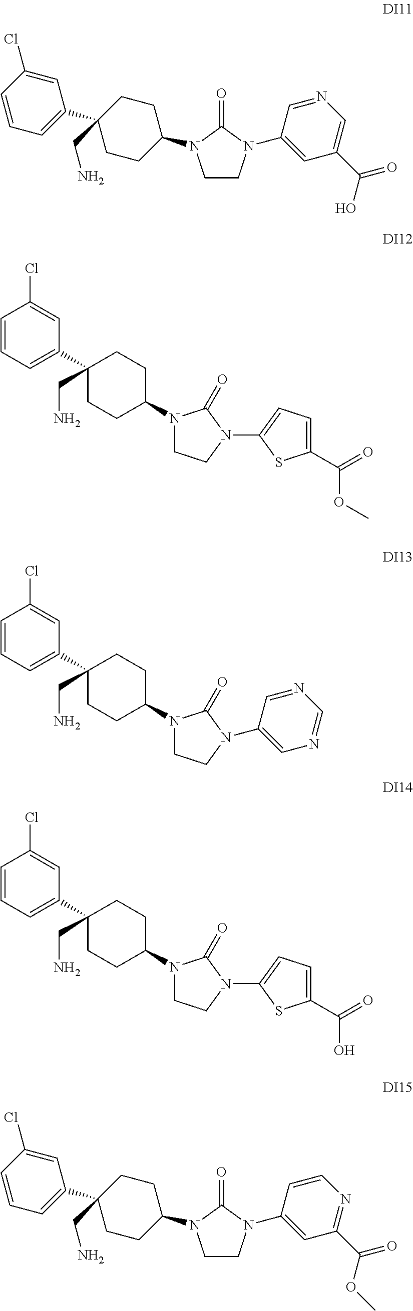 [SMILES: NC[C@]1(c2cccc(Cl)c2)CC[C@H](N2CCN(c3cncc(C(=O)O)c3)C2=O)CC1 ; COC(=O)c1ccc(N2CCN([C@H]3CC[C@](CN)(c4cccc(Cl)c4)CC3)C2=O)s1 ; NC[C@]1(c2cccc(Cl)c2)CC[C@H](N2CCN(c3cncnc3)C2=O)CC1 ; NC[C@]1(c2cccc(Cl)c2)CC[C@H](N2CCN(c3ccc(C(=O)O)s3)C2=O)CC1 ; COC(=O)c1cc(N2CCN([C@H]3CC[C@](CN)(c4cccc(Cl)c4)CC3)C2=O)ccn1]

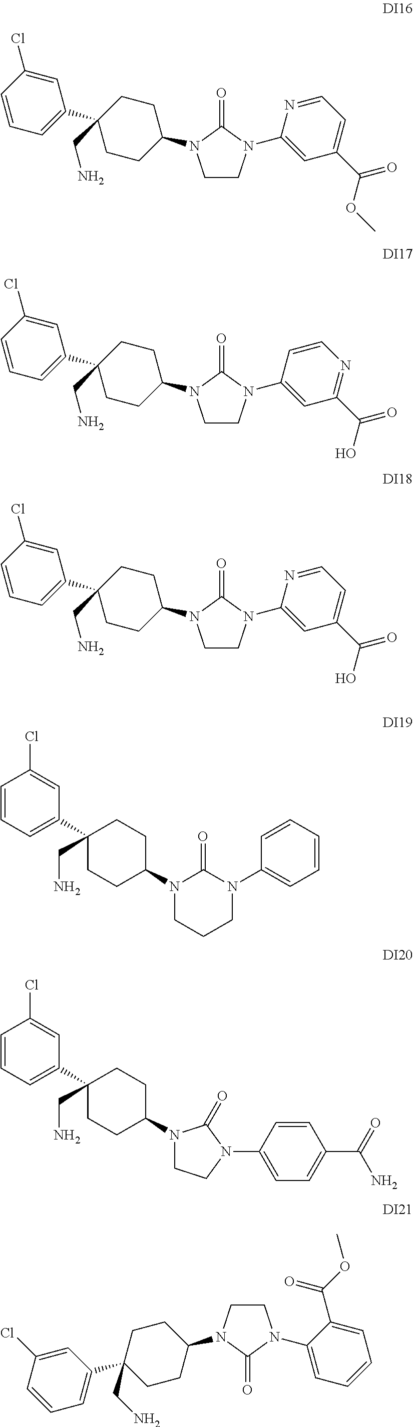 [SMILES: COC(=O)c1ccnc(N2CCN([C@H]3CC[C@](CN)(c4cccc(Cl)c4)CC3)C2=O)c1 ; NC[C@]1(c2cccc(Cl)c2)CC[C@H](N2CCN(c3ccnc(C(=O)O)c3)C2=O)CC1 ; NC[C@]1(c2cccc(Cl)c2)CC[C@H](N2CCN(c3cc(C(=O)O)ccn3)C2=O)CC1 ; NC[C@]1(c2cccc(Cl)c2)CC[C@H](N2CCCN(c3ccccc3)C2=O)CC1 ; NC[C@]1(c2cccc(Cl)c2)CC[C@H](N2CCN(c3ccc(C(N)=O)cc3)C2=O)CC1 ; COC(=O)c1ccccc1N1CCN([C@H]2CC[C@](CN)(c3cccc(Cl)c3)CC2)C1=O]

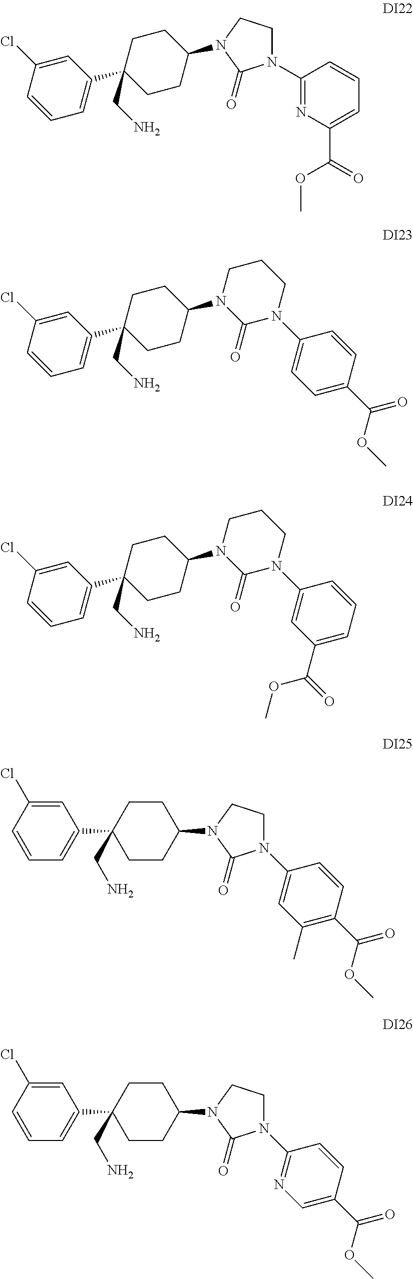 [SMILES: COC(=O)c1cccc(N2CCN([C@H]3CC[C@](CN)(c4cccc(Cl)c4)CC3)C2=O)n1 ; COC(=O)c1ccc(N2CCCN([C@H]3CC[C@](CN)(c4cccc(Cl)c4)CC3)C2=O)cc1 ; COC(=O)c1cccc(N2CCCN([C@H]3CC[C@](CN)(c4cccc(Cl)c4)CC3)C2=O)c1 ; COC(=O)c1ccc(N2CCN([C@H]3CC[C@](CN)(c4cccc(Cl)c4)CC3)C2=O)cc1C ; COC(=O)c1ccc(N2CCN([C@H]3CC[C@](CN)(c4cccc(Cl)c4)CC3)C2=O)nc1]

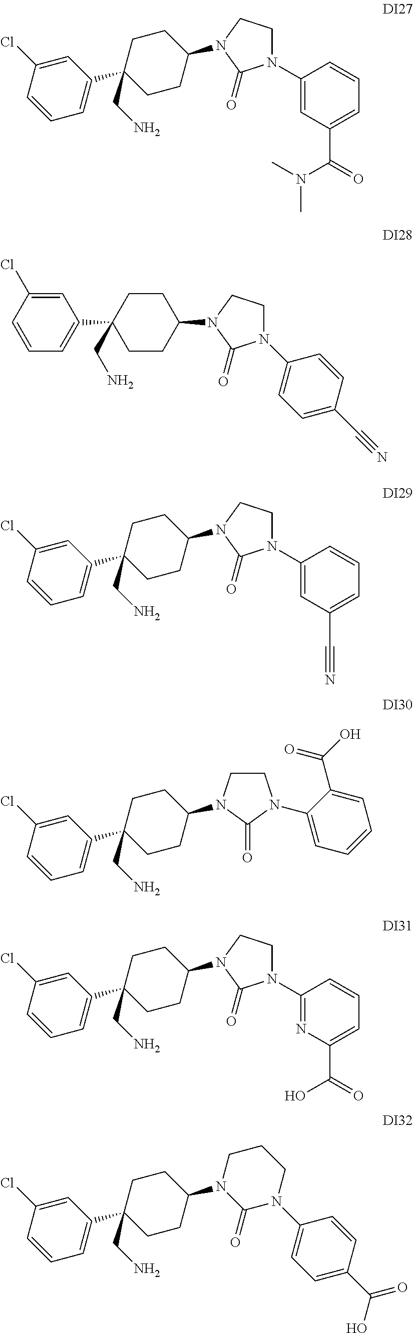 [SMILES: CN(C)C(=O)c1cccc(N2CCN([C@H]3CC[C@](CN)(c4cccc(Cl)c4)CC3)C2=O)c1 ; N#Cc1ccc(N2CCN([C@H]3CC[C@](CN)(c4cccc(Cl)c4)CC3)C2=O)cc1 ; N#Cc1cccc(N2CCN([C@H]3CC[C@](CN)(c4cccc(Cl)c4)CC3)C2=O)c1 ; NC[C@]1(c2cccc(Cl)c2)CC[C@H](N2CCN(c3ccccc3C(=O)O)C2=O)CC1 ; NC[C@]1(c2cccc(Cl)c2)CC[C@H](N2CCN(c3cccc(C(=O)O)n3)C2=O)CC1 ; NC[C@]1(c2cccc(Cl)c2)CC[C@H](N2CCCN(c3ccc(C(=O)O)cc3)C2=O)CC1]

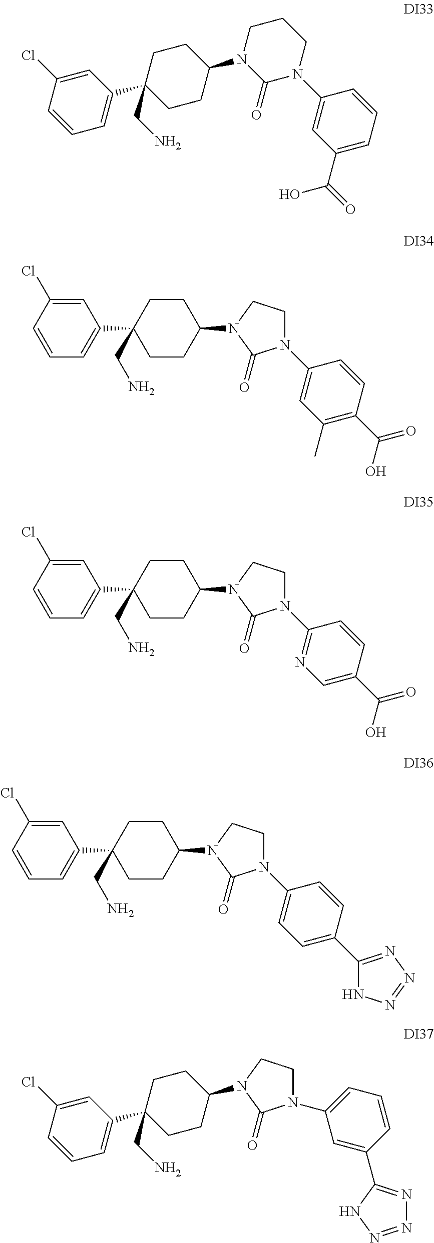 [SMILES: NC[C@]1(c2cccc(Cl)c2)CC[C@H](N2CCCN(c3cccc(C(=O)O)c3)C2=O)CC1 ; Cc1cc(N2CCN([C@H]3CC[C@](CN)(c4cccc(Cl)c4)CC3)C2=O)ccc1C(=O)O ; NC[C@]1(c2cccc(Cl)c2)CC[C@H](N2CCN(c3ccc(C(=O)O)cn3)C2=O)CC1 ; NC[C@]1(c2cccc(Cl)c2)CC[C@H](N2CCN(c3ccc(-c4nnn[nH]4)cc3)C2=O)CC1 ; NC[C@]1(c2cccc(Cl)c2)CC[C@H](N2CCN(c3cccc(-c4nnn[nH]4)c3)C2=O)CC1]

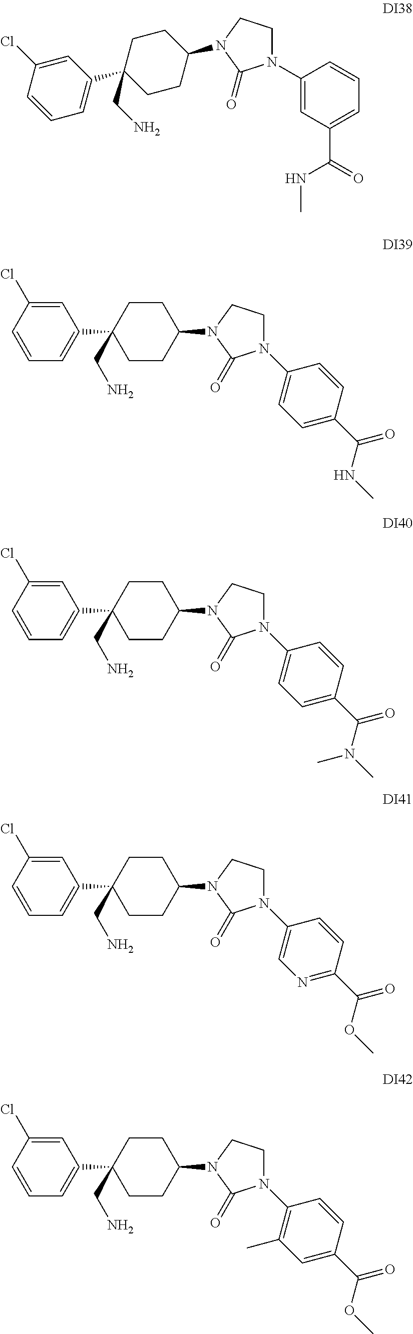 [SMILES: CNC(=O)c1cccc(N2CCN([C@H]3CC[C@](CN)(c4cccc(Cl)c4)CC3)C2=O)c1 ; CNC(=O)c1ccc(N2CCN([C@H]3CC[C@](CN)(c4cccc(Cl)c4)CC3)C2=O)cc1 ; CN(C)C(=O)c1ccc(N2CCN([C@H]3CC[C@](CN)(c4cccc(Cl)c4)CC3)C2=O)cc1 ; COC(=O)c1ccc(N2CCN([C@H]3CC[C@](CN)(c4cccc(Cl)c4)CC3)C2=O)cn1 ; COC(=O)c1ccc(N2CCN([C@H]3CC[C@](CN)(c4cccc(Cl)c4)CC3)C2=O)c(C)c1]

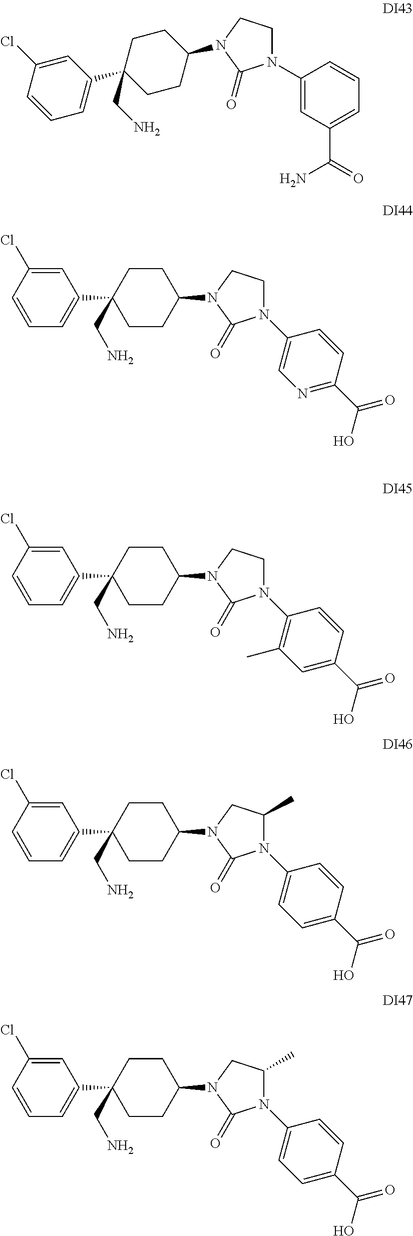 [SMILES: NC[C@]1(c2cccc(Cl)c2)CC[C@H](N2CCN(c3cccc(C(N)=O)c3)C2=O)CC1 ; NC[C@]1(c2cccc(Cl)c2)CC[C@H](N2CCN(c3ccc(C(=O)O)nc3)C2=O)CC1 ; Cc1cc(C(=O)O)ccc1N1CCN([C@H]2CC[C@](CN)(c3cccc(Cl)c3)CC2)C1=O ; C[C@@H]1CN([C@H]2CC[C@](CN)(c3cccc(Cl)c3)CC2)C(=O)N1c1ccc(C(=O)O)cc1 ; C[C@H]1CN([C@H]2CC[C@](CN)(c3cccc(Cl)c3)CC2)C(=O)N1c1ccc(C(=O)O)cc1]

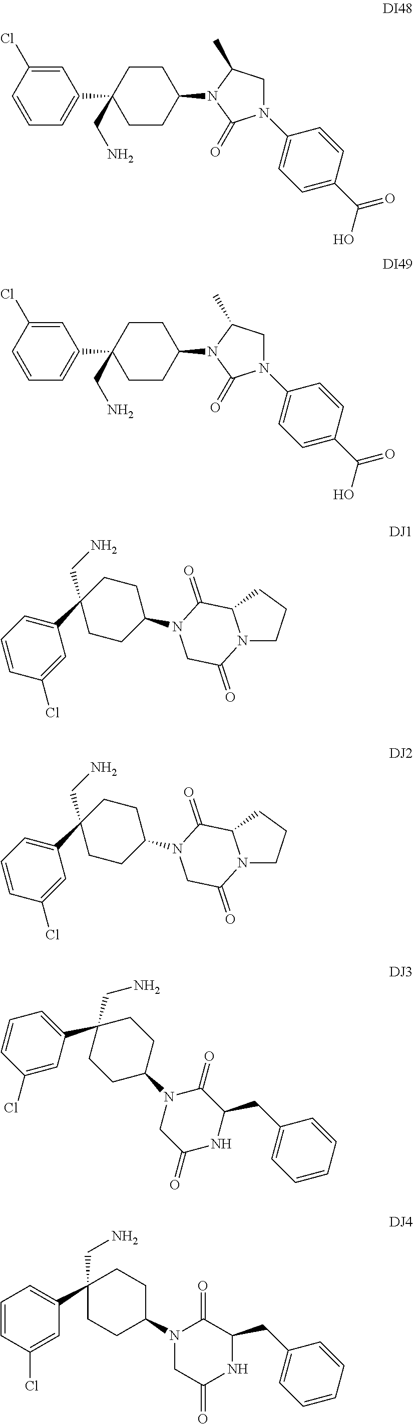 [SMILES: C[C@H]1CN(c2ccc(C(=O)O)cc2)C(=O)N1[C@H]1CC[C@](CN)(c2cccc(Cl)c2)CC1 ; C[C@@H]1CN(c2ccc(C(=O)O)cc2)C(=O)N1[C@H]1CC[C@](CN)(c2cccc(Cl)c2)CC1 ; NC[C@]1(c2cccc(Cl)c2)CC[C@@H](N2CC(=O)N3CCC[C@H]3C2=O)CC1 ; NC[C@]1(c2cccc(Cl)c2)CC[C@H](N2CC(=O)N3CCC[C@H]3C2=O)CC1 ; NC[C@]1(c2cccc(Cl)c2)CC[C@@H](N2CC(=O)N[C@H](Cc3ccccc3)C2=O)CC1 ; NC[C@]1(c2cccc(Cl)c2)CC[C@@H](N2CC(=O)N[C@H](Cc3ccccc3)C2=O)CC1]

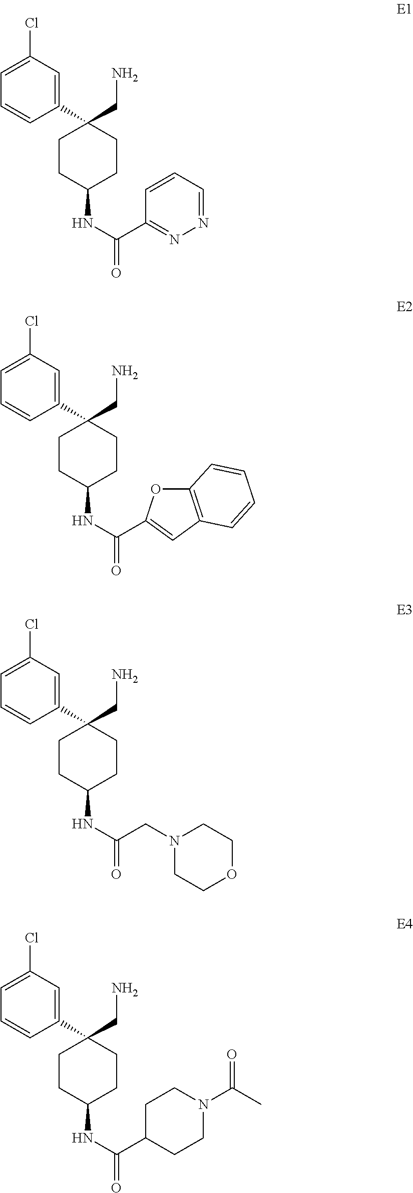 [SMILES: NC[C@]1(c2cccc(Cl)c2)CC[C@H](NC(=O)c2cccnn2)CC1 ; NC[C@]1(c2cccc(Cl)c2)CC[C@H](NC(=O)c2cc3ccccc3o2)CC1 ; NC[C@]1(c2cccc(Cl)c2)CC[C@H](NC(=O)CN2CCOCC2)CC1 ; CC(=O)N1CCC(C(=O)N[C@H]2CC[C@](CN)(c3cccc(Cl)c3)CC2)CC1]

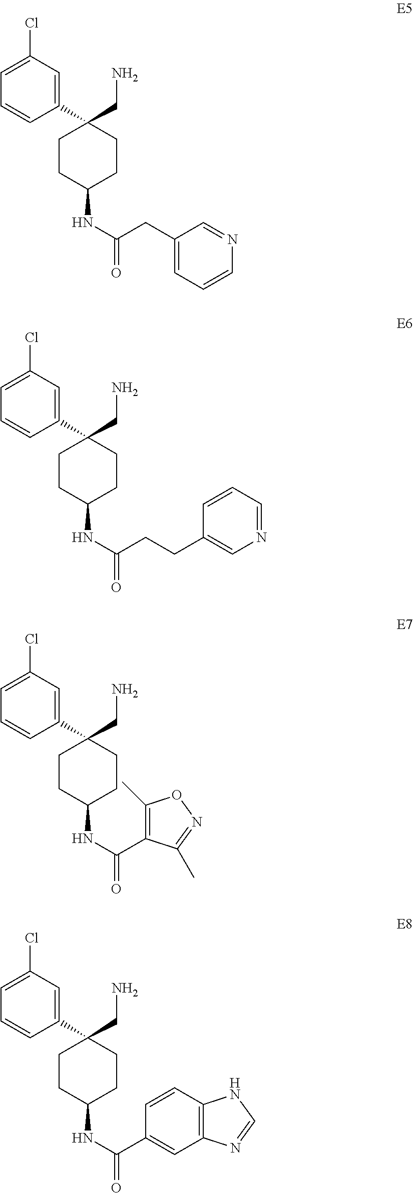 [SMILES: NC[C@]1(c2cccc(Cl)c2)CC[C@H](NC(=O)Cc2cccnc2)CC1 ; NC[C@]1(c2cccc(Cl)c2)CC[C@H](NC(=O)CCc2cccnc2)CC1 ; Cc1noc(C)c1C(=O)N[C@H]1CC[C@](CN)(c2cccc(Cl)c2)CC1 ; NC[C@]1(c2cccc(Cl)c2)CC[C@H](NC(=O)c2ccc3[nH]cnc3c2)CC1]

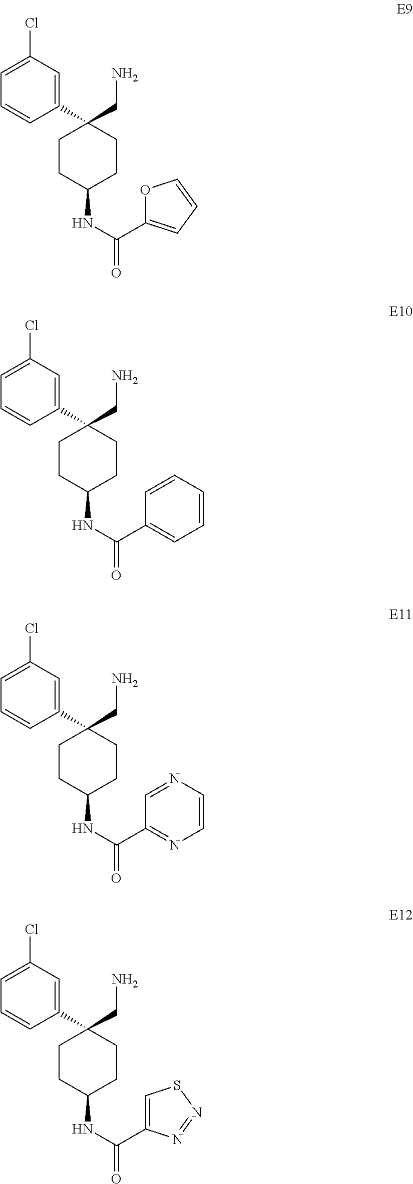 [SMILES: NC[C@]1(c2cccc(Cl)c2)CC[C@H](NC(=O)c2ccco2)CC1 ; NC[C@]1(c2cccc(Cl)c2)CC[C@H](NC(=O)c2ccccc2)CC1 ; NC[C@]1(c2cccc(Cl)c2)CC[C@H](NC(=O)c2cnccn2)CC1 ; NC[C@]1(c2cccc(Cl)c2)CC[C@H](NC(=O)c2csnn2)CC1]

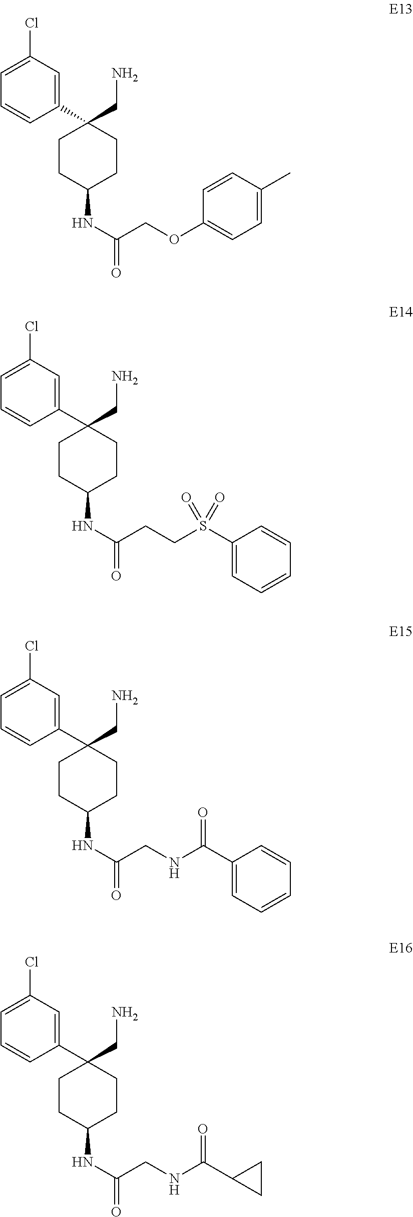 [SMILES: Cc1ccc(OCC(=O)N[C@H]2CC[C@](CN)(c3cccc(Cl)c3)CC2)cc1 ; NC[C@]1(c2cccc(Cl)c2)CC[C@H](NC(=O)CCS(=O)(=O)c2ccccc2)CC1 ; NC[C@]1(c2cccc(Cl)c2)CC[C@H](NC(=O)CNC(=O)c2ccccc2)CC1 ; NC[C@]1(c2cccc(Cl)c2)CC[C@H](NC(=O)CNC(=O)C2CC2)CC1]

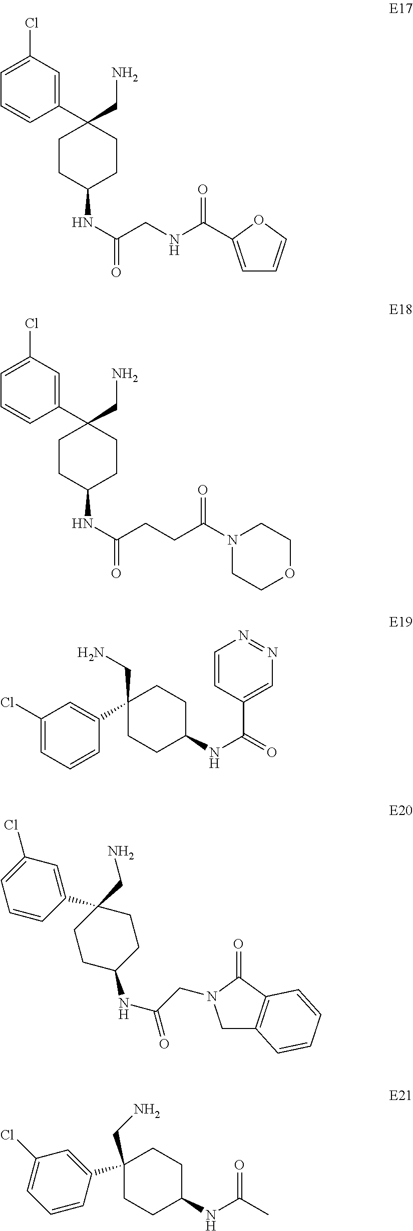 [SMILES: NC[C@]1(c2cccc(Cl)c2)CC[C@H](NC(=O)CNC(=O)c2ccco2)CC1 ; NC[C@]1(c2cccc(Cl)c2)CC[C@H](NC(=O)CCC(=O)N2CCOCC2)CC1 ; NC[C@]1(c2cccc(Cl)c2)CC[C@H](NC(=O)c2ccnnc2)CC1 ; NC[C@]1(c2cccc(Cl)c2)CC[C@H](NC(=O)CN2Cc3ccccc3C2=O)CC1 ; CC(=O)N[C@H]1CC[C@](CN)(c2cccc(Cl)c2)CC1]

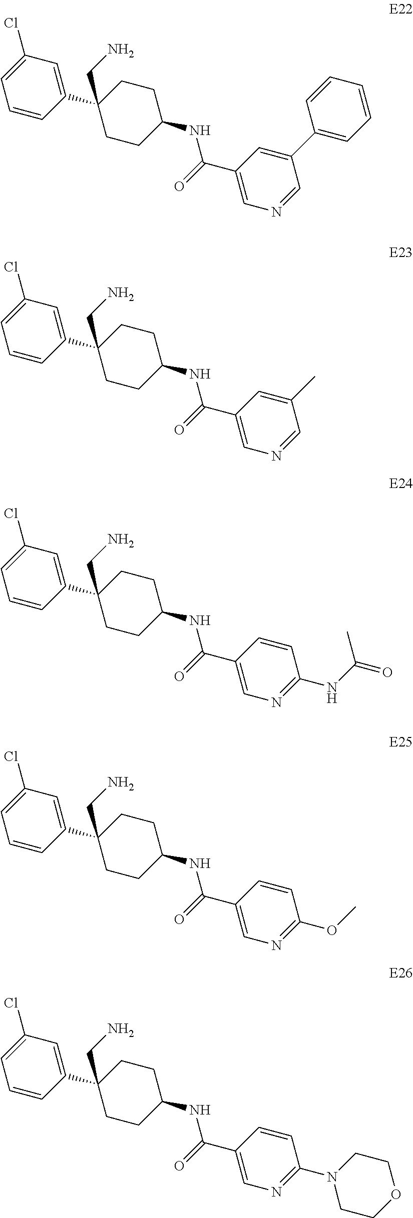 [SMILES: NC[C@]1(c2cccc(Cl)c2)CC[C@H](NC(=O)c2cncc(-c3ccccc3)c2)CC1 ; Cc1cncc(C(=O)N[C@H]2CC[C@](CN)(c3cccc(Cl)c3)CC2)c1 ; CC(=O)Nc1ccc(C(=O)N[C@H]2CC[C@](CN)(c3cccc(Cl)c3)CC2)cn1 ; COc1ccc(C(=O)N[C@H]2CC[C@](CN)(c3cccc(Cl)c3)CC2)cn1 ; NC[C@]1(c2cccc(Cl)c2)CC[C@H](NC(=O)c2ccc(N3CCOCC3)nc2)CC1]

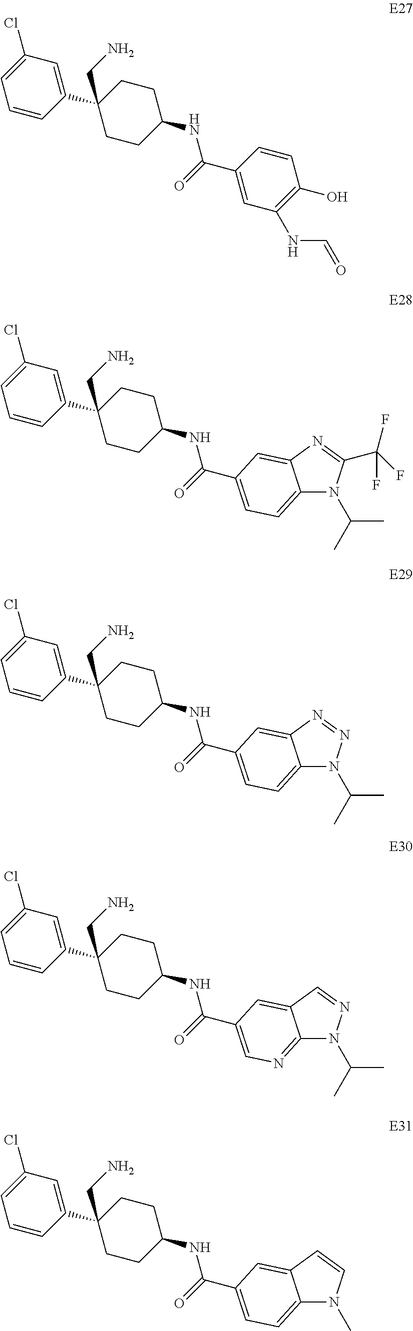 [SMILES: NC[C@]1(c2cccc(Cl)c2)CC[C@H](NC(=O)c2ccc(O)c(NC=O)c2)CC1 ; CC(C)n1c(C(F)(F)F)nc2cc(C(=O)N[C@H]3CC[C@](CN)(c4cccc(Cl)c4)CC3)ccc21 ; CC(C)n1nnc2cc(C(=O)N[C@H]3CC[C@](CN)(c4cccc(Cl)c4)CC3)ccc21 ; CC(C)n1ncc2cc(C(=O)N[C@H]3CC[C@](CN)(c4cccc(Cl)c4)CC3)cnc21 ; Cn1ccc2cc(C(=O)N[C@H]3CC[C@](CN)(c4cccc(Cl)c4)CC3)ccc21]

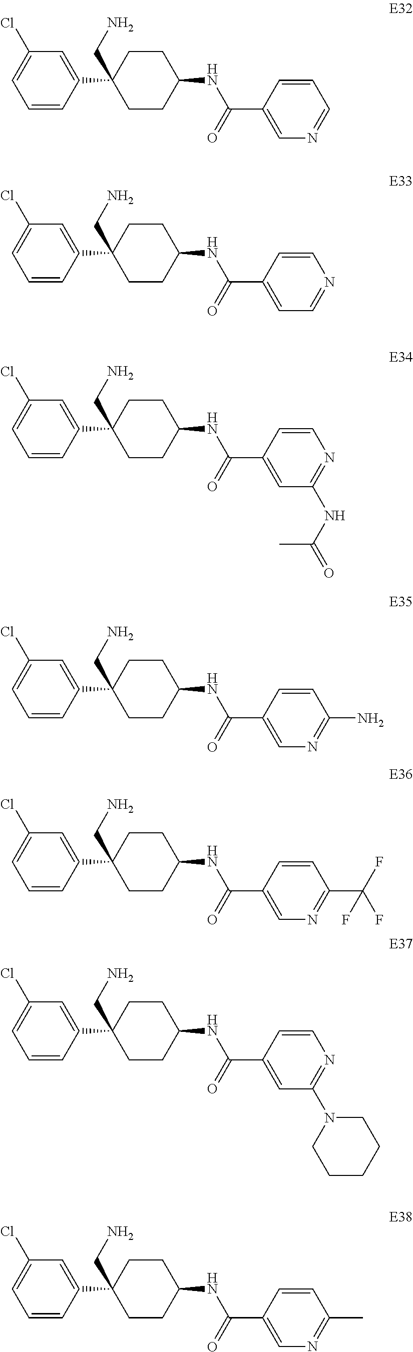

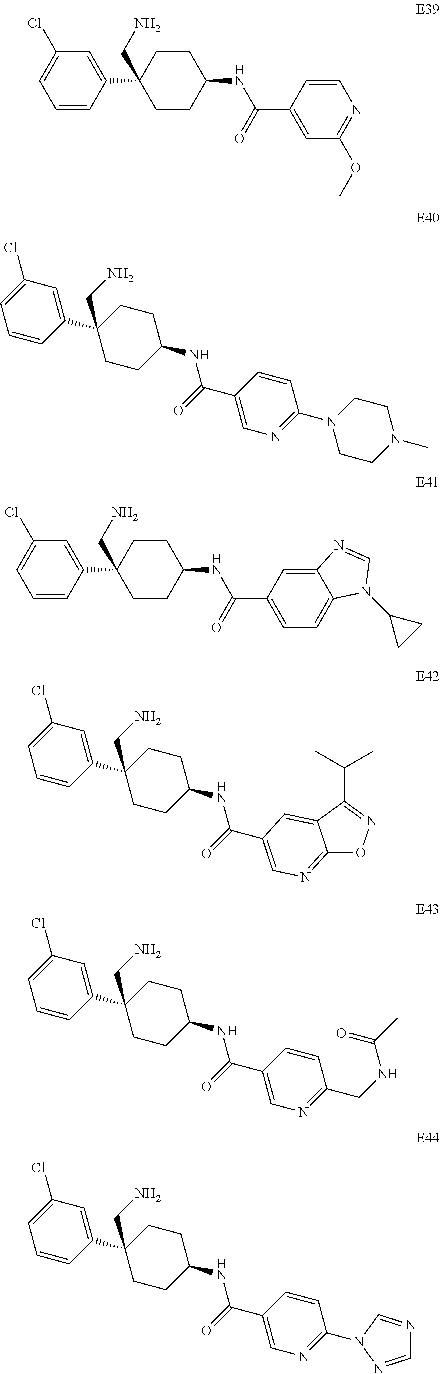 [SMILES: COc1cc(C(=O)N[C@H]2CC[C@](CN)(c3cccc(Cl)c3)CC2)ccn1 ; CN1CCN(c2ccc(C(=O)N[C@H]3CC[C@](CN)(c4cccc(Cl)c4)CC3)cn2)CC1 ; NC[C@]1(c2cccc(Cl)c2)CC[C@H](NC(=O)c2ccc3c(c2)ncn3C2CC2)CC1 ; CC(C)c1noc2ncc(C(=O)N[C@H]3CC[C@](CN)(c4cccc(Cl)c4)CC3)cc12 ; CC(=O)NCc1ccc(C(=O)N[C@H]2CC[C@](CN)(c3cccc(Cl)c3)CC2)cn1 ; NC[C@]1(c2cccc(Cl)c2)CC[C@H](NC(=O)c2ccc(-n3cncn3)nc2)CC1]

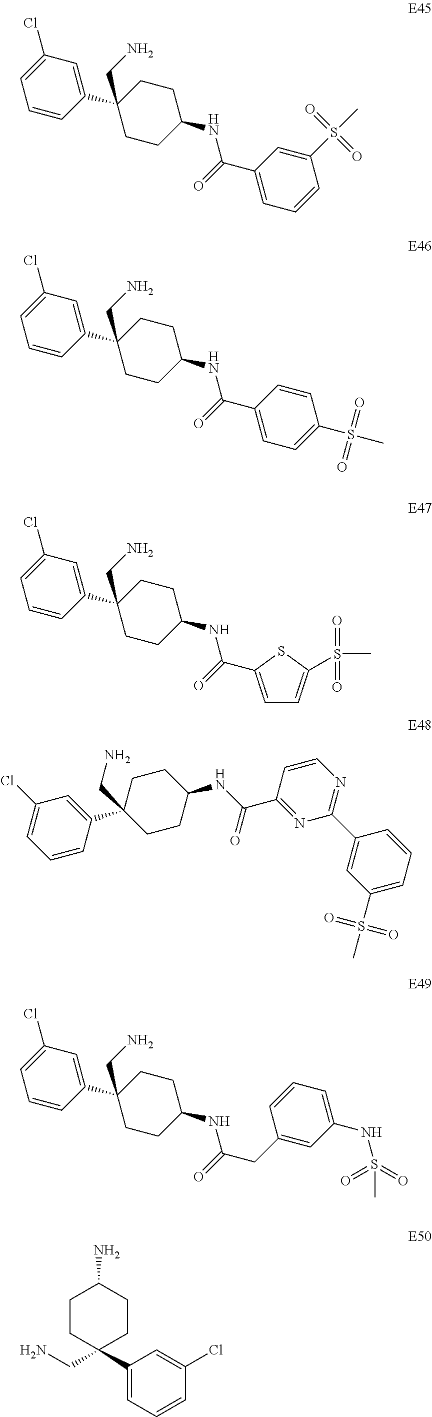 [SMILES: CS(=O)(=O)c1cccc(C(=O)N[C@H]2CC[C@](CN)(c3cccc(Cl)c3)CC2)c1 ; CS(=O)(=O)c1ccc(C(=O)N[C@H]2CC[C@](CN)(c3cccc(Cl)c3)CC2)cc1 ; CS(=O)(=O)c1ccc(C(=O)N[C@H]2CC[C@](CN)(c3cccc(Cl)c3)CC2)s1 ; CS(=O)(=O)c1cccc(-c2nccc(C(=O)N[C@H]3CC[C@](CN)(c4cccc(Cl)c4)CC3)n2)c1 ; CS(=O)(=O)Nc1cccc(CC(=O)N[C@H]2CC[C@](CN)(c3cccc(Cl)c3)CC2)c1 ; NC[C@]1(c2cccc(Cl)c2)CC[C@H](N)CC1]

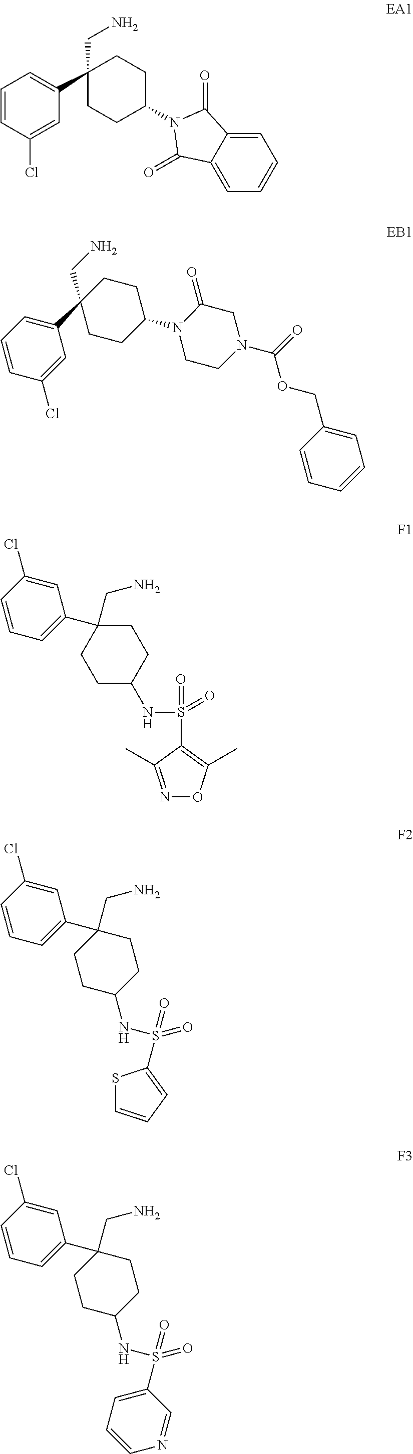 [SMILES: NC[C@]1(c2cccc(Cl)c2)CC[C@H](N2C(=O)c3ccccc3C2=O)CC1 ; NC[C@]1(c2cccc(Cl)c2)CC[C@H](N2CCN(C(=O)OCc3ccccc3)CC2=O)CC1 ; Cc1noc(C)c1S(=O)(=O)NC1CCC(CN)(c2cccc(Cl)c2)CC1 ; NCC1(c2cccc(Cl)c2)CCC(NS(=O)(=O)c2cccs2)CC1 ; NCC1(c2cccc(Cl)c2)CCC(NS(=O)(=O)c2cccnc2)CC1]

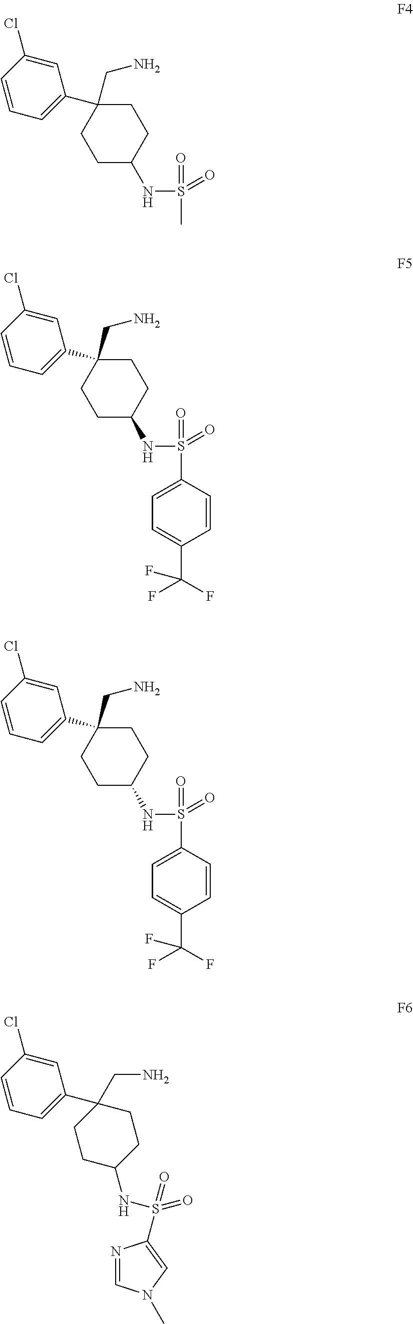 [SMILES: CS(=O)(=O)NC1CCC(CN)(c2cccc(Cl)c2)CC1 ; NC[C@]1(c2cccc(Cl)c2)CC[C@H](NS(=O)(=O)c2ccc(C(F)(F)F)cc2)CC1 ; NC[C@]1(c2cccc(Cl)c2)CC[C@@H](NS(=O)(=O)c2ccc(C(F)(F)F)cc2)CC1 ; Cn1cnc(S(=O)(=O)NC2CCC(CN)(c3cccc(Cl)c3)CC2)c1]

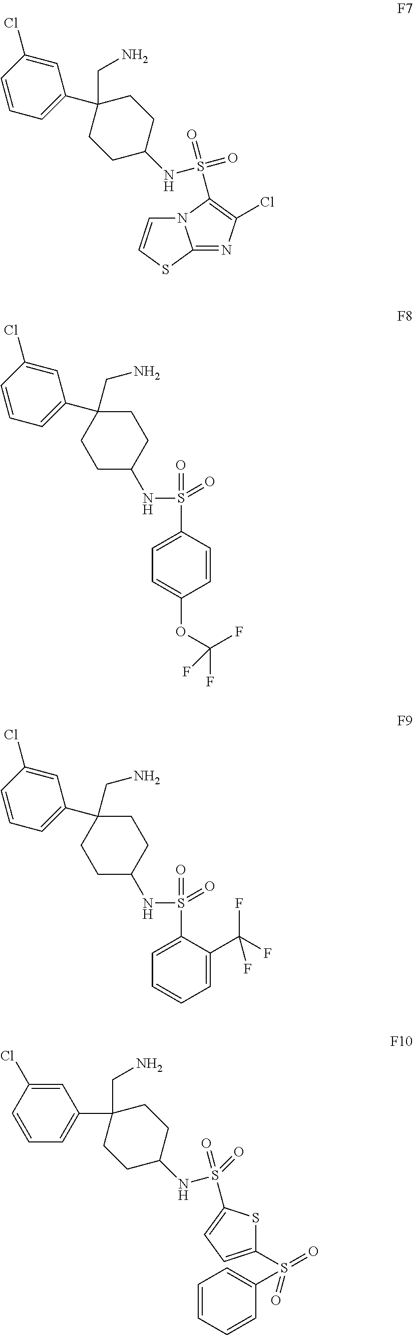 [SMILES: NCC1(c2cccc(Cl)c2)CCC(NS(=O)(=O)c2c(Cl)nc3sccn23)CC1 ; NCC1(c2cccc(Cl)c2)CCC(NS(=O)(=O)c2ccc(OC(F)(F)F)cc2)CC1 ; NCC1(c2cccc(Cl)c2)CCC(NS(=O)(=O)c2ccccc2C(F)(F)F)CC1 ; NCC1(c2cccc(Cl)c2)CCC(NS(=O)(=O)c2ccc(S(=O)(=O)c3ccccc3)s2)CC1]

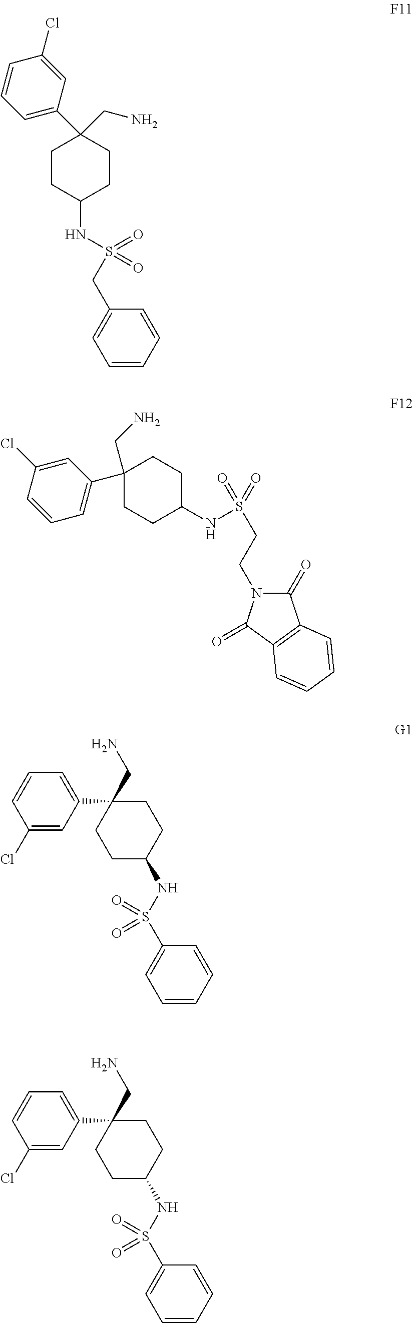 [SMILES: NCC1(c2cccc(Cl)c2)CCC(NS(=O)(=O)Cc2ccccc2)CC1 ; NCC1(c2cccc(Cl)c2)CCC(NS(=O)(=O)CCN2C(=O)c3ccccc3C2=O)CC1 ; NC[C@]1(c2cccc(Cl)c2)CC[C@H](NS(=O)(=O)c2ccccc2)CC1 ; NC[C@]1(c2cccc(Cl)c2)CC[C@@H](NS(=O)(=O)c2ccccc2)CC1]

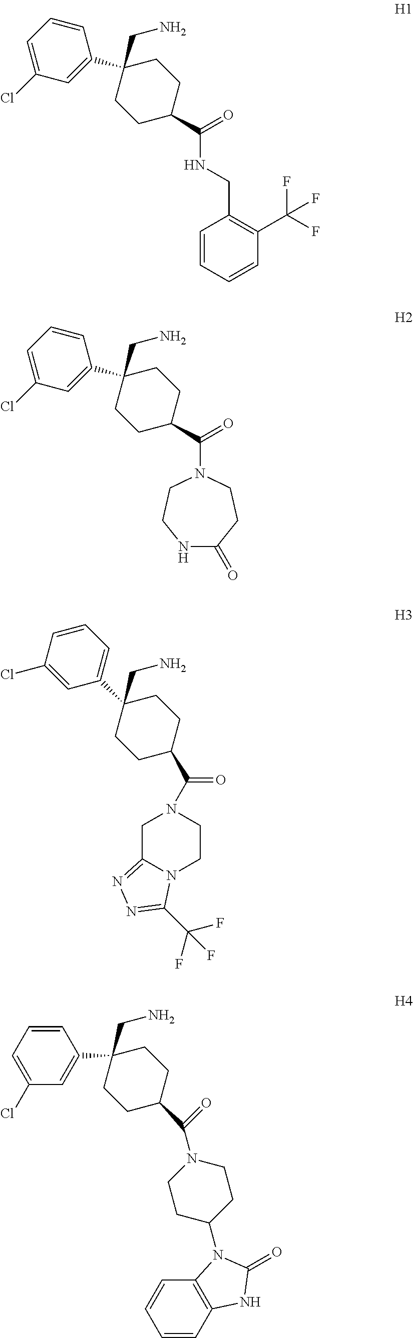 [SMILES: NC[C@]1(c2cccc(Cl)c2)CC[C@H](C(=O)NCc2ccccc2C(F)(F)F)CC1 ; NC[C@]1(c2cccc(Cl)c2)CC[C@H](C(=O)N2CCNC(=O)CC2)CC1 ; NC[C@]1(c2cccc(Cl)c2)CC[C@H](C(=O)N2CCn3c(nnc3C(F)(F)F)C2)CC1 ; NC[C@]1(c2cccc(Cl)c2)CC[C@H](C(=O)N2CCC(n3c(=O)[nH]c4ccccc43)CC2)CC1]

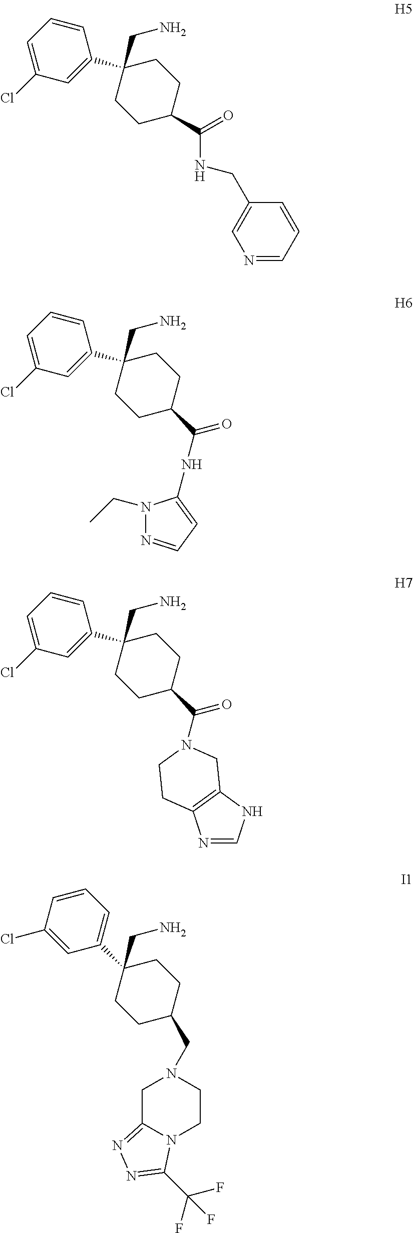 [SMILES: NC[C@]1(c2cccc(Cl)c2)CC[C@H](C(=O)NCc2cccnc2)CC1 ; CCn1nccc1NC(=O)[C@H]1CC[C@](CN)(c2cccc(Cl)c2)CC1 ; NC[C@]1(c2cccc(Cl)c2)CC[C@H](C(=O)N2CCc3nc[nH]c3C2)CC1 ; NC[C@]1(c2cccc(Cl)c2)CC[C@H](CN2CCn3c(nnc3C(F)(F)F)C2)CC1]

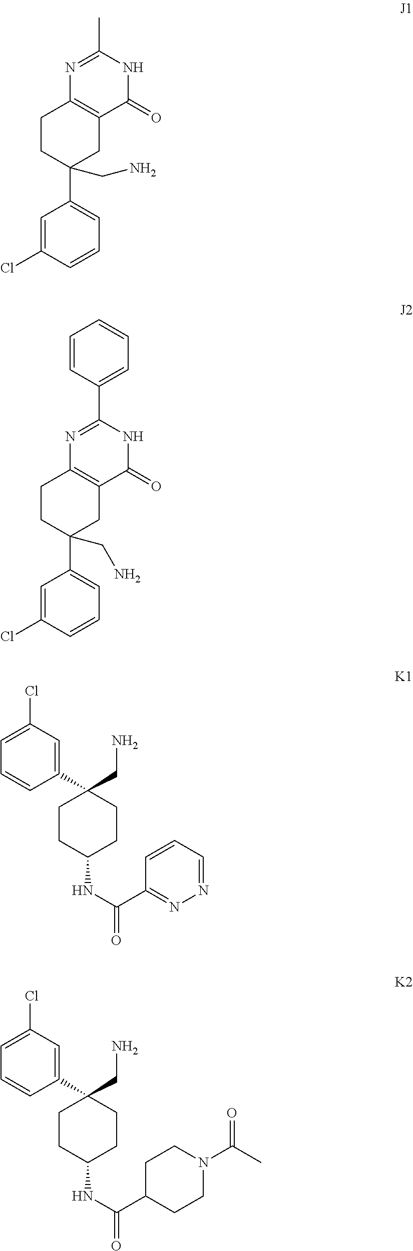 [SMILES: Cc1nc2c(c(=O)[nH]1)CC(CN)(c1cccc(Cl)c1)CC2 ; NCC1(c2cccc(Cl)c2)CCc2nc(-c3ccccc3)[nH]c(=O)c2C1 ; NC[C@]1(c2cccc(Cl)c2)CC[C@@H](NC(=O)c2cccnn2)CC1 ; CC(=O)N1CCC(C(=O)N[C@H]2CC[C@@](CN)(c3cccc(Cl)c3)CC2)CC1]

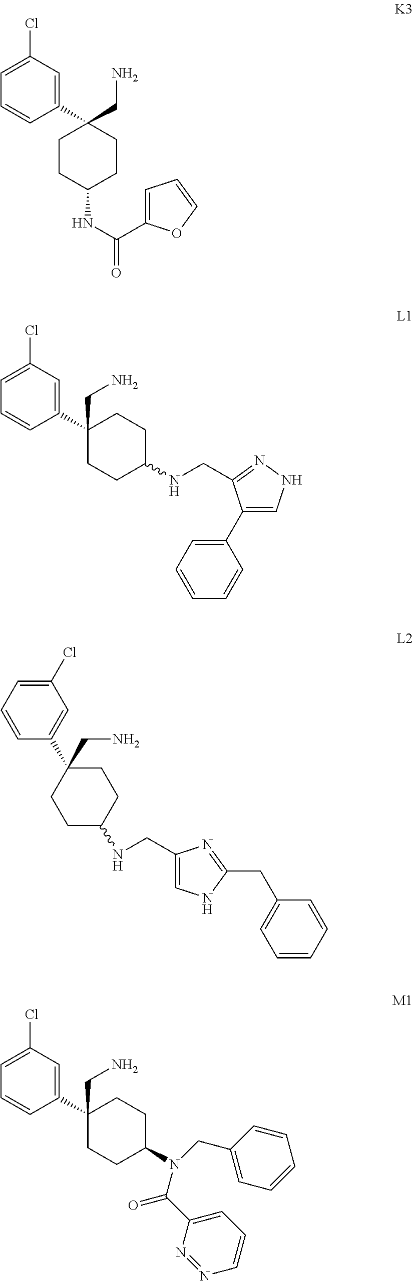

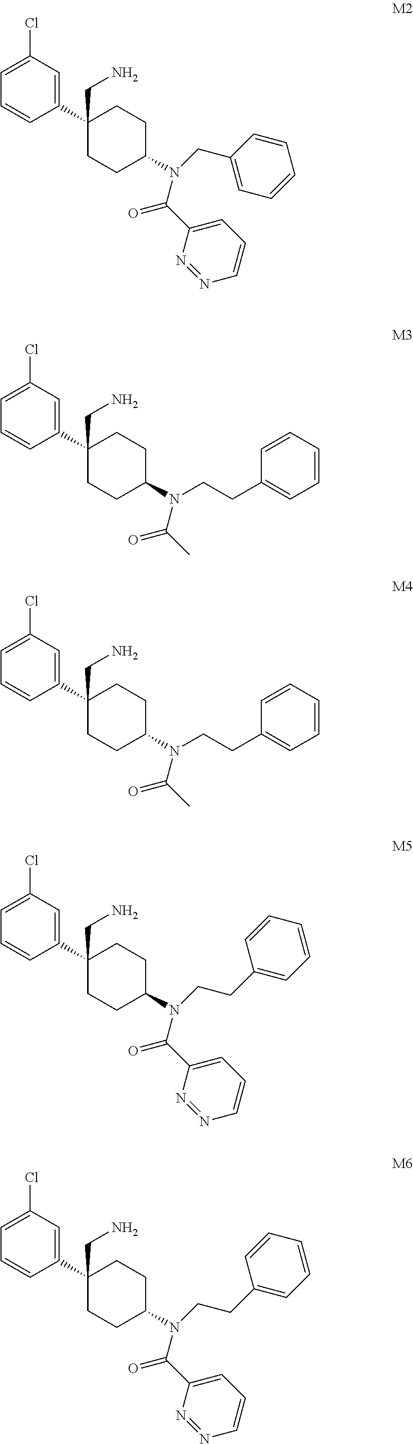 [SMILES: NC[C@]1(c2cccc(Cl)c2)CC[C@@H](N(Cc2ccccc2)C(=O)c2cccnn2)CC1 ; CC(=O)N(CCc1ccccc1)[C@H]1CC[C@](CN)(c2cccc(Cl)c2)CC1 ; CC(=O)N(CCc1ccccc1)[C@H]1CC[C@@](CN)(c2cccc(Cl)c2)CC1 ; NC[C@]1(c2cccc(Cl)c2)CC[C@H](N(CCc2ccccc2)C(=O)c2cccnn2)CC1 ; NC[C@]1(c2cccc(Cl)c2)CC[C@@H](N(CCc2ccccc2)C(=O)c2cccnn2)CC1]

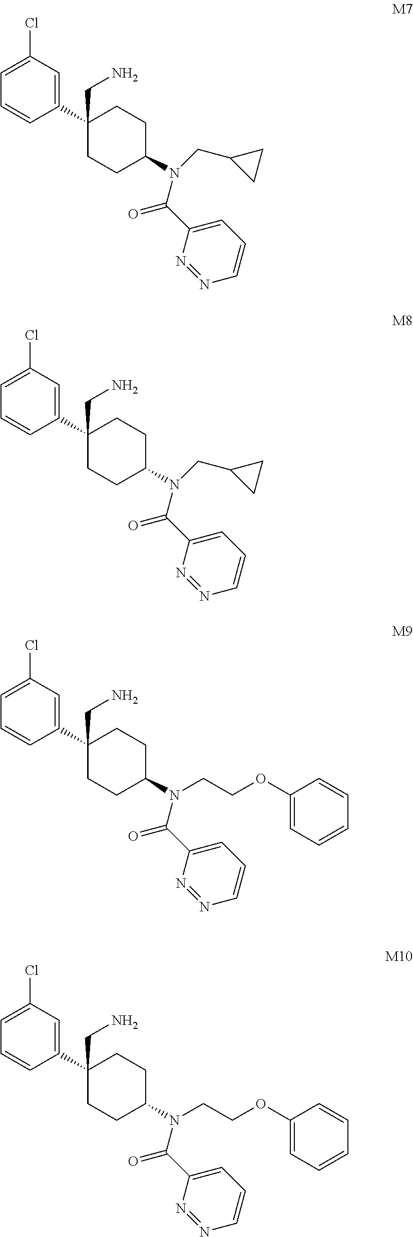 [SMILES: NC[C@]1(c2cccc(Cl)c2)CC[C@H](N(CC2CC2)C(=O)c2cccnn2)CC1 ; NC[C@]1(c2cccc(Cl)c2)CC[C@@H](N(CC2CC2)C(=O)c2cccnn2)CC1 ; NC[C@]1(c2cccc(Cl)c2)CC[C@H](N(CCOc2ccccc2)C(=O)c2cccnn2)CC1 ; NC[C@]1(c2cccc(Cl)c2)CC[C@@H](N(CCOc2ccccc2)C(=O)c2cccnn2)CC1]

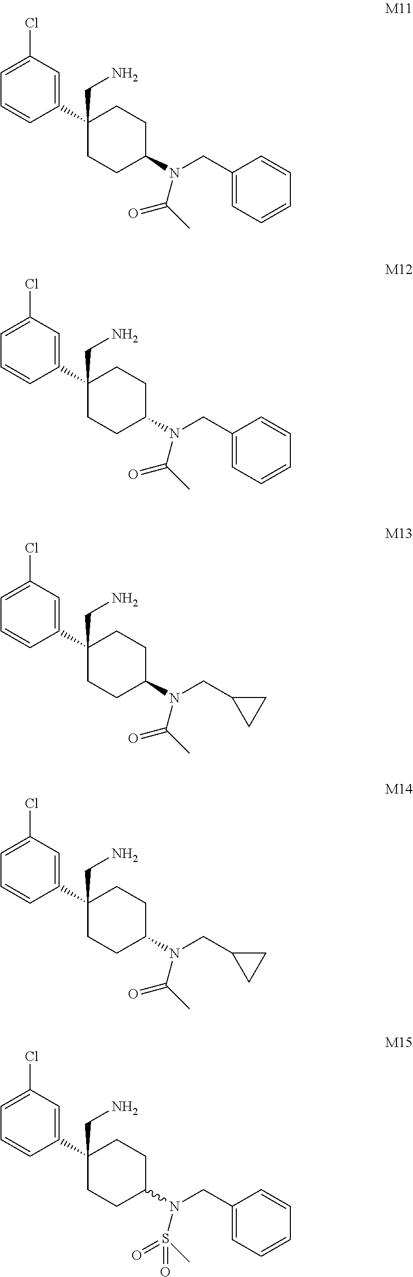

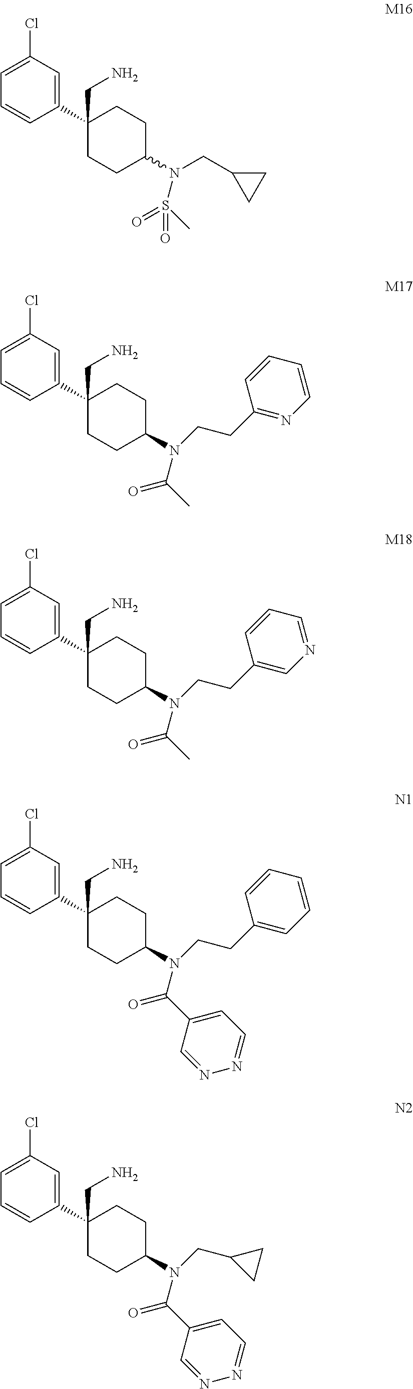

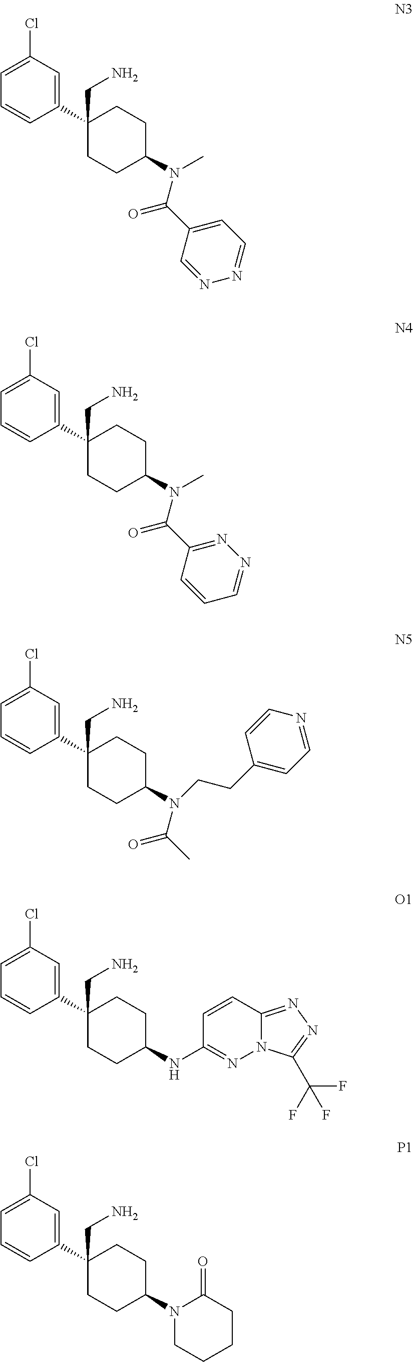 [SMILES: CN(C(=O)c1ccnnc1)[C@H]1CC[C@](CN)(c2cccc(Cl)c2)CC1 ; CN(C(=O)c1cccnn1)[C@H]1CC[C@](CN)(c2cccc(Cl)c2)CC1 ; CC(=O)N(CCc1ccncc1)[C@H]1CC[C@](CN)(c2cccc(Cl)c2)CC1 ; NC[C@]1(c2cccc(Cl)c2)CC[C@H](Nc2ccc3nnc(C(F)(F)F)n3n2)CC1 ; NC[C@]1(c2cccc(Cl)c2)CC[C@H](N2CCCCC2=O)CC1]

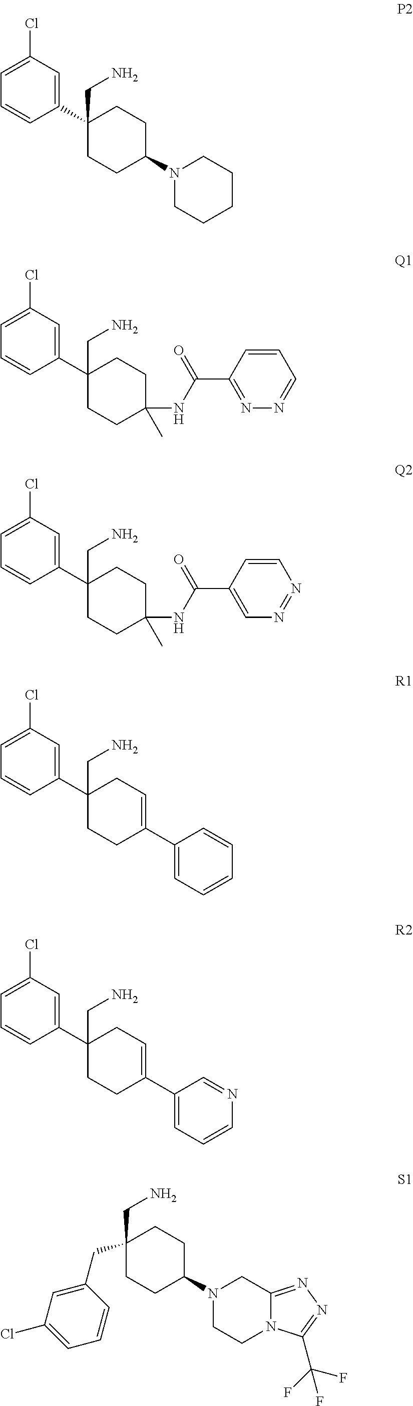 [SMILES: NC[C@]1(c2cccc(Cl)c2)CC[C@H](N2CCCCC2)CC1 ; CC1(NC(=O)c2cccnn2)CCC(CN)(c2cccc(Cl)c2)CC1 ; CC1(NC(=O)c2ccnnc2)CCC(CN)(c2cccc(Cl)c2)CC1 ; NCC1(c2cccc(Cl)c2)CC=C(c2ccccc2)CC1 ; NCC1(c2cccc(Cl)c2)CC=C(c2cccnc2)CC1 ; NC[C@]1(Cc2cccc(Cl)c2)CC[C@H](N2CCn3c(nnc3C(F)(F)F)C2)CC1]

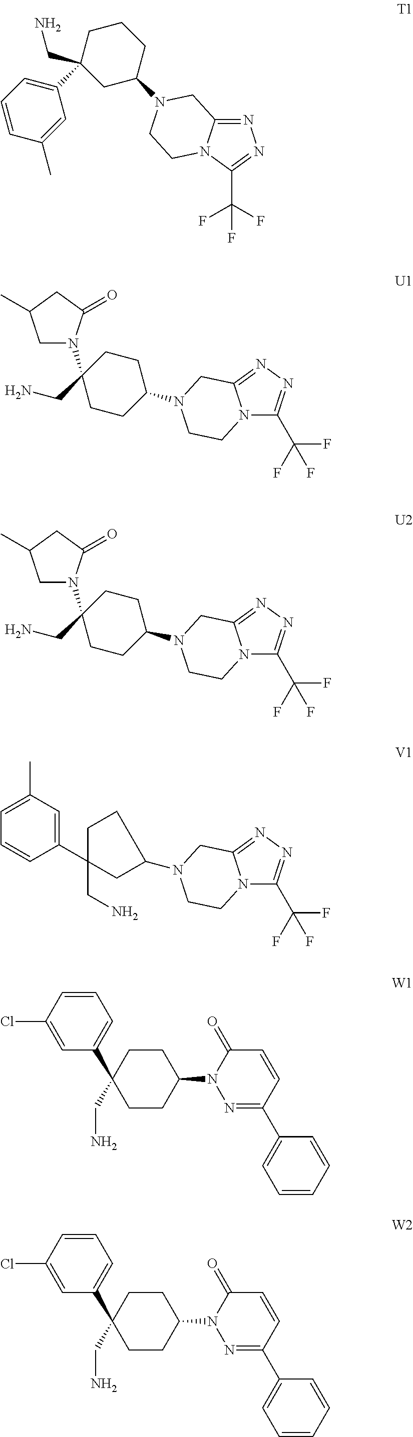 [SMILES: Cc1cccc([C@]2(CN)CCC[C@@H](N3CCn4c(nnc4C(F)(F)F)C3)C2)c1 ; CC1CC(=O)N([C@]2(CN)CC[C@H](N3CCn4c(nnc4C(F)(F)F)C3)CC2)C1 ; CC1CC(=O)N([C@]2(CN)CC[C@@H](N3CCn4c(nnc4C(F)(F)F)C3)CC2)C1 ; Cc1cccc(C2(CN)CCC(N3CCn4c(nnc4C(F)(F)F)C3)C2)c1 ; NC[C@]1(c2cccc(Cl)c2)CC[C@@H](n2nc(-c3ccccc3)ccc2=O)CC1 ; NC[C@]1(c2cccc(Cl)c2)CC[C@H](n2nc(-c3ccccc3)ccc2=O)CC1]

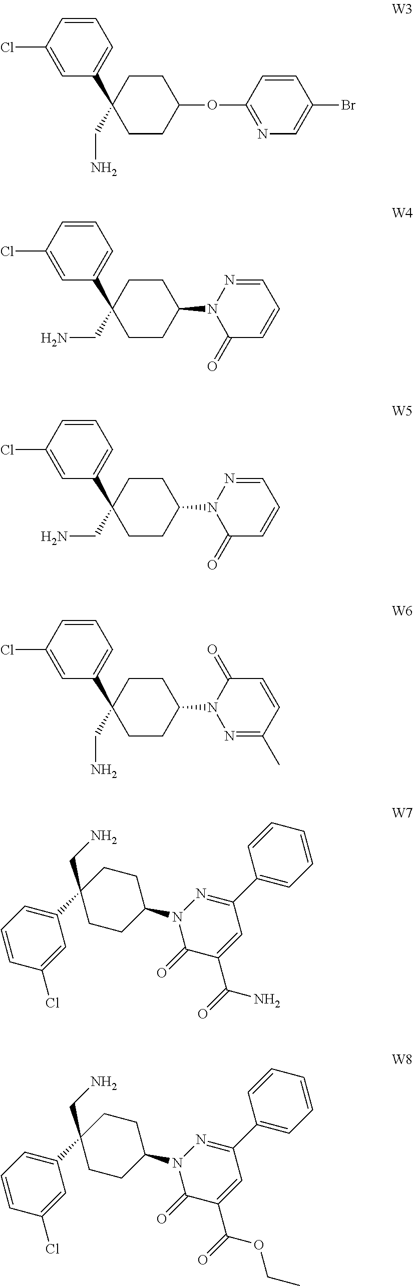

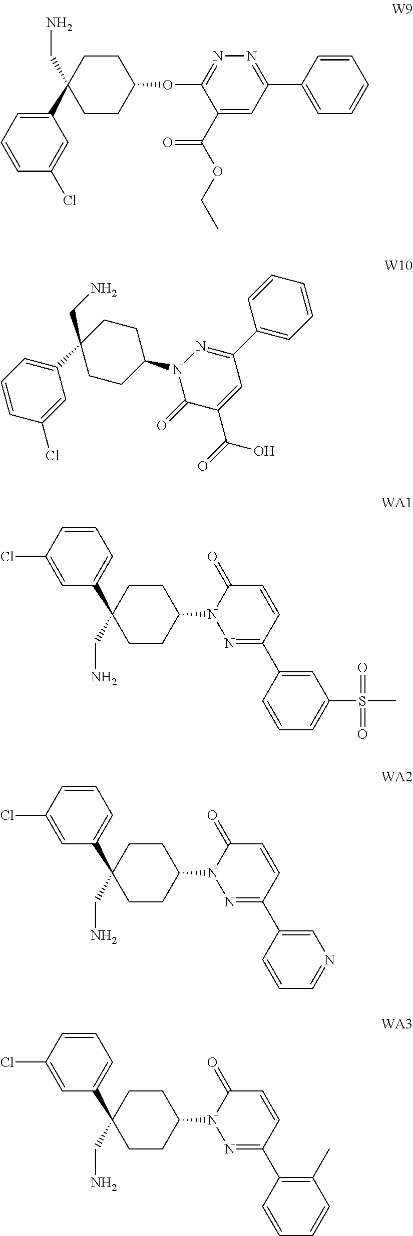 [SMILES: CCOC(=O)c1cc(-c2ccccc2)nnc1O[C@H]1CC[C@](CN)(c2cccc(Cl)c2)CC1 ; NC[C@]1(c2cccc(Cl)c2)CC[C@H](n2nc(-c3ccccc3)cc(C(=O)O)c2=O)CC1 ; CS(=O)(=O)c1cccc(-c2ccc(=O)n([C@H]3CC[C@](CN)(c4cccc(Cl)c4)CC3)n2)c1 ; NC[C@]1(c2cccc(Cl)c2)CC[C@H](n2nc(-c3cccnc3)ccc2=O)CC1 ; Cc1ccccc1-c1ccc(=O)n([C@H]2CC[C@](CN)(c3cccc(Cl)c3)CC2)n1]

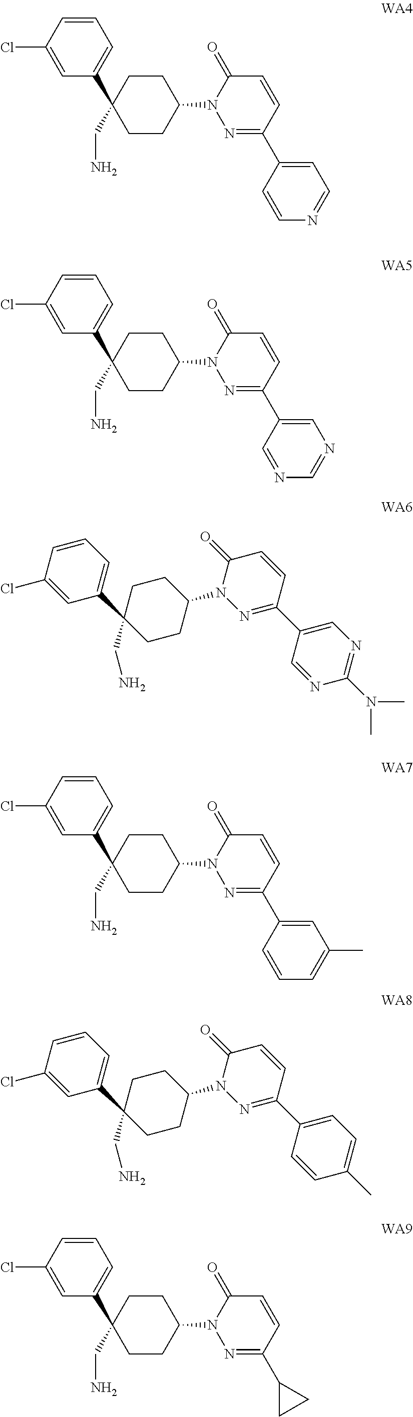 [SMILES: NC[C@]1(c2cccc(Cl)c2)CC[C@H](n2nc(-c3ccncc3)ccc2=O)CC1 ; NC[C@]1(c2cccc(Cl)c2)CC[C@H](n2nc(-c3cncnc3)ccc2=O)CC1 ; CN(C)c1ncc(-c2ccc(=O)n([C@H]3CC[C@](CN)(c4cccc(Cl)c4)CC3)n2)cn1 ; Cc1cccc(-c2ccc(=O)n([C@H]3CC[C@](CN)(c4cccc(Cl)c4)CC3)n2)c1 ; Cc1ccc(-c2ccc(=O)n([C@H]3CC[C@](CN)(c4cccc(Cl)c4)CC3)n2)cc1 ; NC[C@]1(c2cccc(Cl)c2)CC[C@H](n2nc(C3CC3)ccc2=O)CC1]

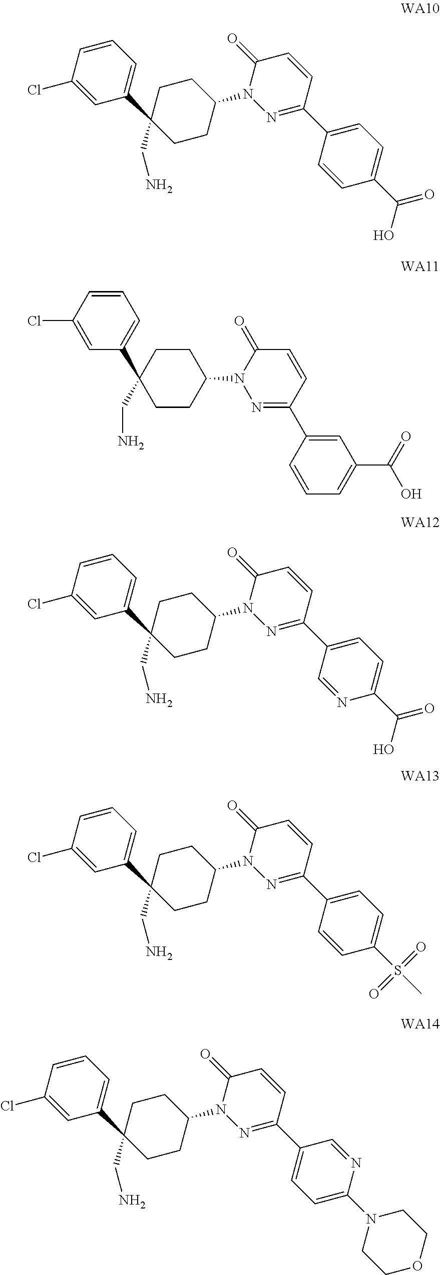 [SMILES: NC[C@]1(c2cccc(Cl)c2)CC[C@H](n2nc(-c3ccc(C(=O)O)cc3)ccc2=O)CC1 ; NC[C@]1(c2cccc(Cl)c2)CC[C@H](n2nc(-c3cccc(C(=O)O)c3)ccc2=O)CC1 ; NC[C@]1(c2cccc(Cl)c2)CC[C@H](n2nc(-c3ccc(C(=O)O)nc3)ccc2=O)CC1 ; CS(=O)(=O)c1ccc(-c2ccc(=O)n([C@H]3CC[C@](CN)(c4cccc(Cl)c4)CC3)n2)cc1 ; NC[C@]1(c2cccc(Cl)c2)CC[C@H](n2nc(-c3ccc(N4CCOCC4)nc3)ccc2=O)CC1]

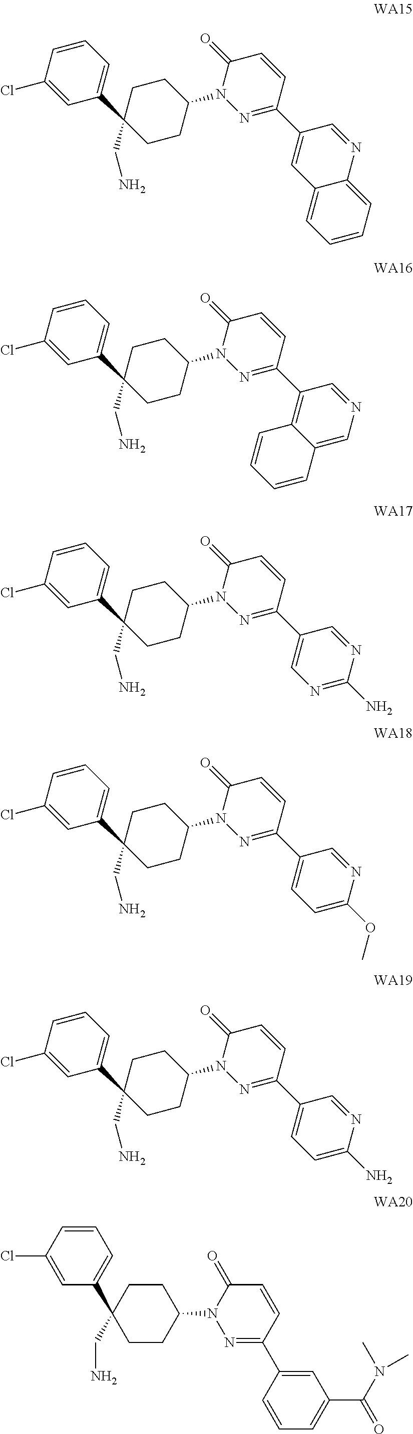 [SMILES: NC[C@]1(c2cccc(Cl)c2)CC[C@H](n2nc(-c3cnc4ccccc4c3)ccc2=O)CC1 ; NC[C@]1(c2cccc(Cl)c2)CC[C@H](n2nc(-c3cncc4ccccc34)ccc2=O)CC1 ; NC[C@]1(c2cccc(Cl)c2)CC[C@H](n2nc(-c3cnc(N)nc3)ccc2=O)CC1 ; COc1ccc(-c2ccc(=O)n([C@H]3CC[C@](CN)(c4cccc(Cl)c4)CC3)n2)cn1 ; NC[C@]1(c2cccc(Cl)c2)CC[C@H](n2nc(-c3ccc(N)nc3)ccc2=O)CC1 ; CN(C)C(=O)c1cccc(-c2ccc(=O)n([C@H]3CC[C@](CN)(c4cccc(Cl)c4)CC3)n2)c1]

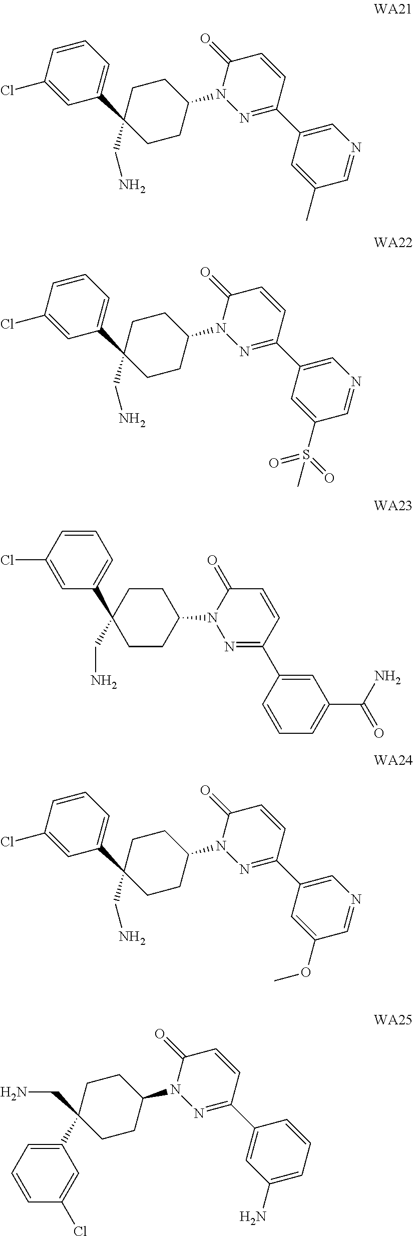 [SMILES: Cc1cncc(-c2ccc(=O)n([C@H]3CC[C@](CN)(c4cccc(Cl)c4)CC3)n2)c1 ; CS(=O)(=O)c1cncc(-c2ccc(=O)n([C@H]3CC[C@](CN)(c4cccc(Cl)c4)CC3)n2)c1 ; NC[C@]1(c2cccc(Cl)c2)CC[C@H](n2nc(-c3cccc(C(N)=O)c3)ccc2=O)CC1 ; COc1cncc(-c2ccc(=O)n([C@H]3CC[C@](CN)(c4cccc(Cl)c4)CC3)n2)c1 ; NC[C@]1(c2cccc(Cl)c2)CC[C@H](n2nc(-c3cccc(N)c3)ccc2=O)CC1]

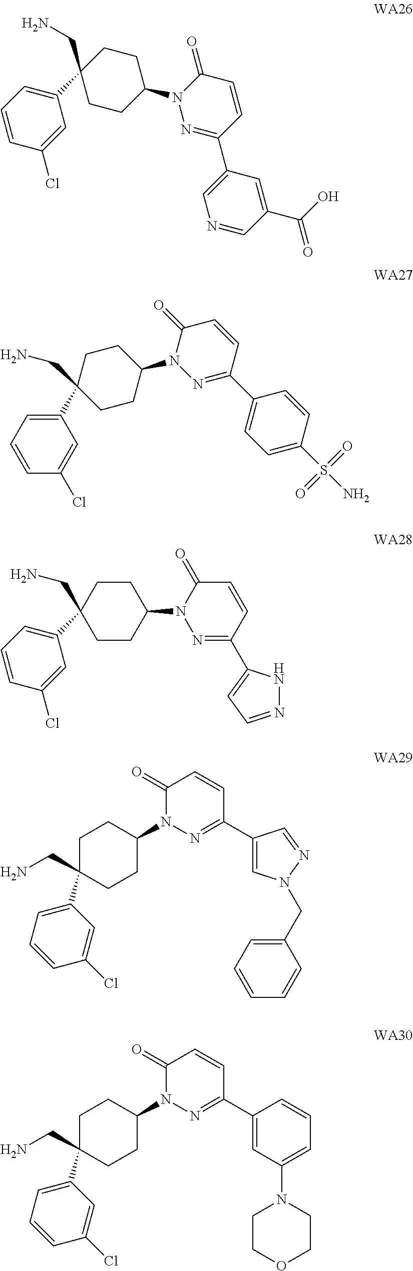 [SMILES: NC[C@]1(c2cccc(Cl)c2)CC[C@H](n2nc(-c3cncc(C(=O)O)c3)ccc2=O)CC1 ; NC[C@]1(c2cccc(Cl)c2)CC[C@H](n2nc(-c3ccc(S(N)(=O)=O)cc3)ccc2=O)CC1 ; NC[C@]1(c2cccc(Cl)c2)CC[C@H](n2nc(-c3ccn[nH]3)ccc2=O)CC1 ; NC[C@]1(c2cccc(Cl)c2)CC[C@H](n2nc(-c3cnn(Cc4ccccc4)c3)ccc2=O)CC1 ; NC[C@]1(c2cccc(Cl)c2)CC[C@H](n2nc(-c3cccc(N4CCOCC4)c3)ccc2=O)CC1]

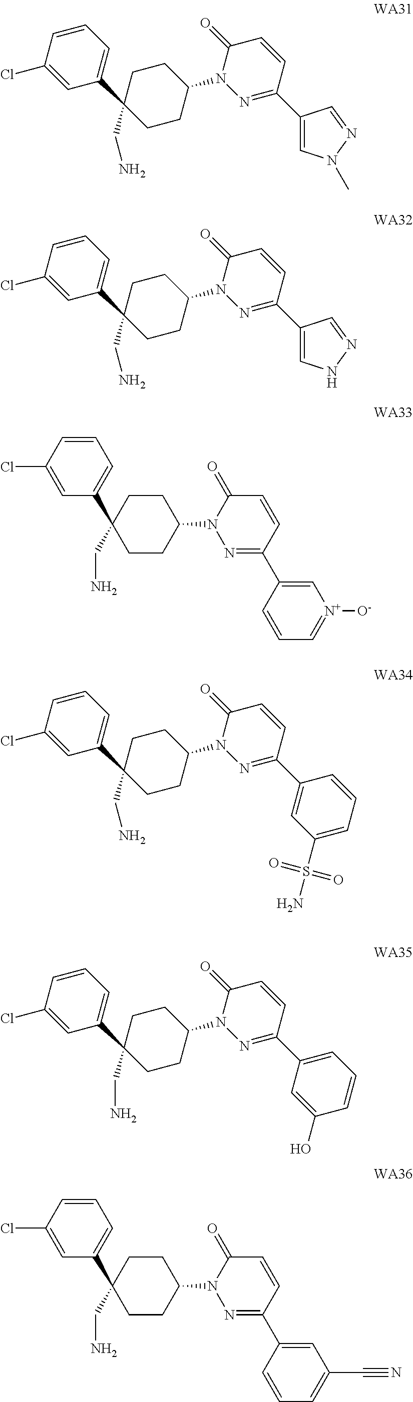 [SMILES: Cn1cc(-c2ccc(=O)n([C@H]3CC[C@](CN)(c4cccc(Cl)c4)CC3)n2)cn1 ; NC[C@]1(c2cccc(Cl)c2)CC[C@H](n2nc(-c3cn[nH]c3)ccc2=O)CC1 ; NC[C@]1(c2cccc(Cl)c2)CC[C@H](n2nc(-c3ccc[n+]([O-])c3)ccc2=O)CC1 ; NC[C@]1(c2cccc(Cl)c2)CC[C@H](n2nc(-c3cccc(S(N)(=O)=O)c3)ccc2=O)CC1 ; NC[C@]1(c2cccc(Cl)c2)CC[C@H](n2nc(-c3cccc(O)c3)ccc2=O)CC1 ; N#Cc1cccc(-c2ccc(=O)n([C@H]3CC[C@](CN)(c4cccc(Cl)c4)CC3)n2)c1]

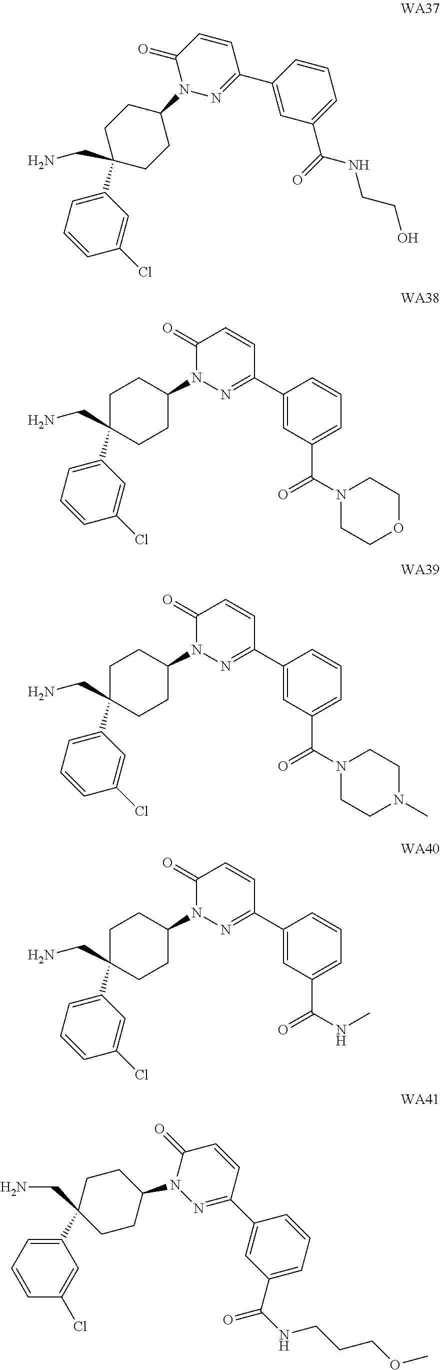 [SMILES: NC[C@]1(c2cccc(Cl)c2)CC[C@H](n2nc(-c3cccc(C(=O)NCCO)c3)ccc2=O)CC1 ; NC[C@]1(c2cccc(Cl)c2)CC[C@H](n2nc(-c3cccc(C(=O)N4CCOCC4)c3)ccc2=O)CC1 ; CN1CCN(C(=O)c2cccc(-c3ccc(=O)n([C@H]4CC[C@](CN)(c5cccc(Cl)c5)CC4)n3)c2)CC1 ; CNC(=O)c1cccc(-c2ccc(=O)n([C@H]3CC[C@](CN)(c4cccc(Cl)c4)CC3)n2)c1 ; COCCCNC(=O)c1cccc(-c2ccc(=O)n([C@H]3CC[C@](CN)(c4cccc(Cl)c4)CC3)n2)c1]

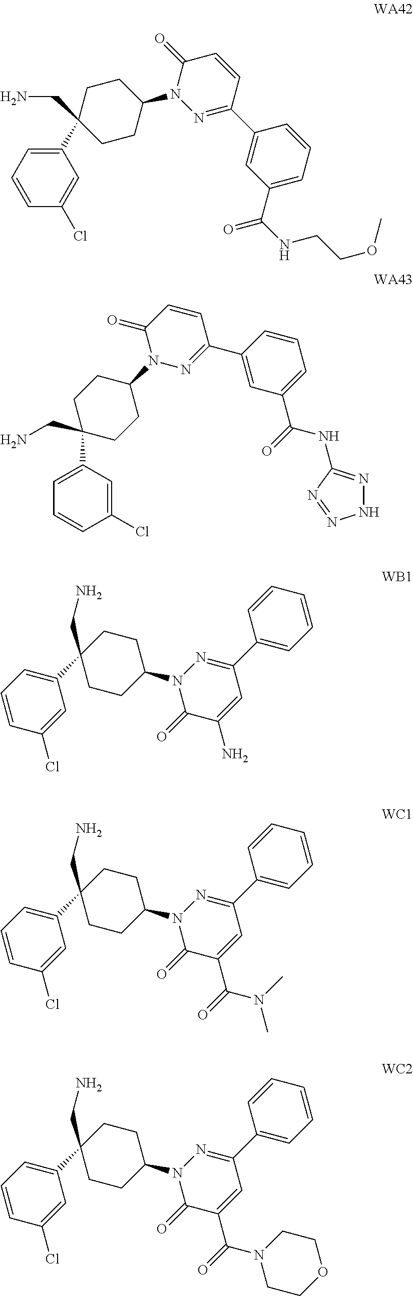 [SMILES: COCCNC(=O)c1cccc(-c2ccc(=O)n([C@H]3CC[C@](CN)(c4cccc(Cl)c4)CC3)n2)c1 ; NC[C@]1(c2cccc(Cl)c2)CC[C@H](n2nc(-c3cccc(C(=O)Nc4nn[nH]n4)c3)ccc2=O)CC1 ; NC[C@]1(c2cccc(Cl)c2)CC[C@H](n2nc(-c3ccccc3)cc(N)c2=O)CC1 ; CN(C)C(=O)c1cc(-c2ccccc2)nn([C@H]2CC[C@](CN)(c3cccc(Cl)c3)CC2)c1=O ; NC[C@]1(c2cccc(Cl)c2)CC[C@H](n2nc(-c3ccccc3)cc(C(=O)N3CCOCC3)c2=O)CC1]

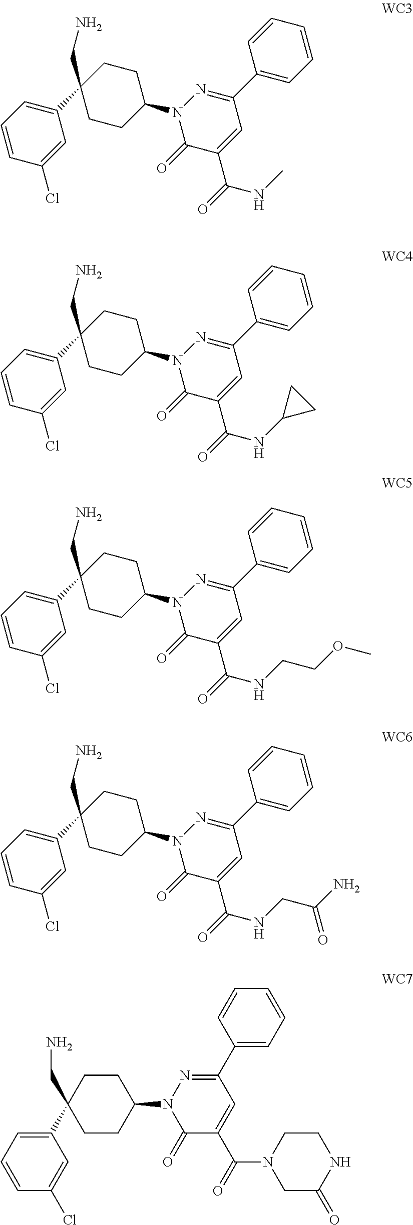 [SMILES: CNC(=O)c1cc(-c2ccccc2)nn([C@H]2CC[C@](CN)(c3cccc(Cl)c3)CC2)c1=O ; NC[C@]1(c2cccc(Cl)c2)CC[C@H](n2nc(-c3ccccc3)cc(C(=O)NC3CC3)c2=O)CC1 ; COCCNC(=O)c1cc(-c2ccccc2)nn([C@H]2CC[C@](CN)(c3cccc(Cl)c3)CC2)c1=O ; NC[C@]1(c2cccc(Cl)c2)CC[C@H](n2nc(-c3ccccc3)cc(C(=O)NCC(N)=O)c2=O)CC1 ; NC[C@]1(c2cccc(Cl)c2)CC[C@H](n2nc(-c3ccccc3)cc(C(=O)N3CCNC(=O)C3)c2=O)CC1]

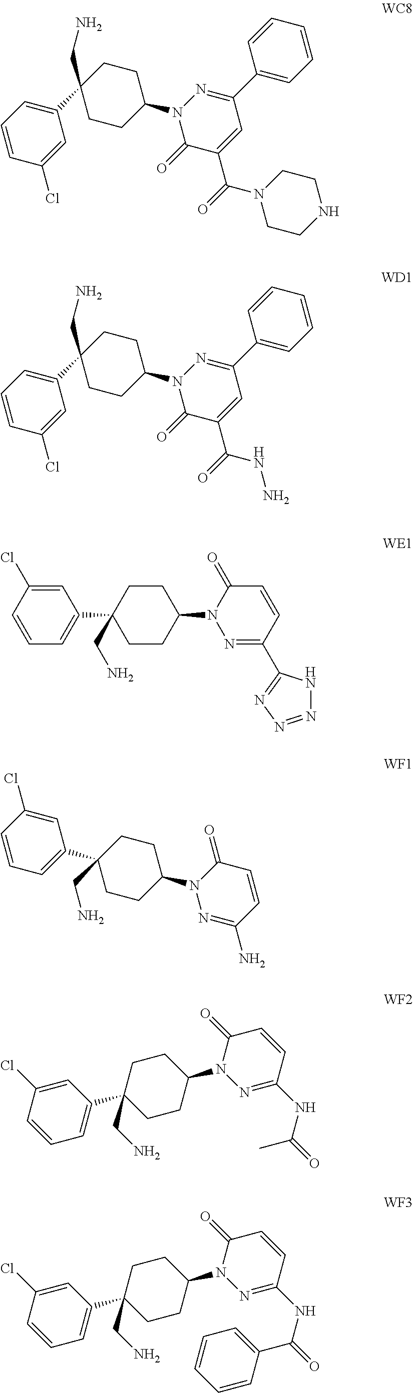 [SMILES: NC[C@]1(c2cccc(Cl)c2)CC[C@H](n2nc(-c3ccccc3)cc(C(=O)N3CCNCC3)c2=O)CC1 ; NC[C@]1(c2cccc(Cl)c2)CC[C@H](n2nc(-c3ccccc3)cc(C(=O)NN)c2=O)CC1 ; NC[C@]1(c2cccc(Cl)c2)CC[C@H](n2nc(-c3nnn[nH]3)ccc2=O)CC1 ; NC[C@]1(c2cccc(Cl)c2)CC[C@H](n2nc(N)ccc2=O)CC1 ; CC(=O)Nc1ccc(=O)n([C@H]2CC[C@](CN)(c3cccc(Cl)c3)CC2)n1 ; NC[C@]1(c2cccc(Cl)c2)CC[C@H](n2nc(NC(=O)c3ccccc3)ccc2=O)CC1]

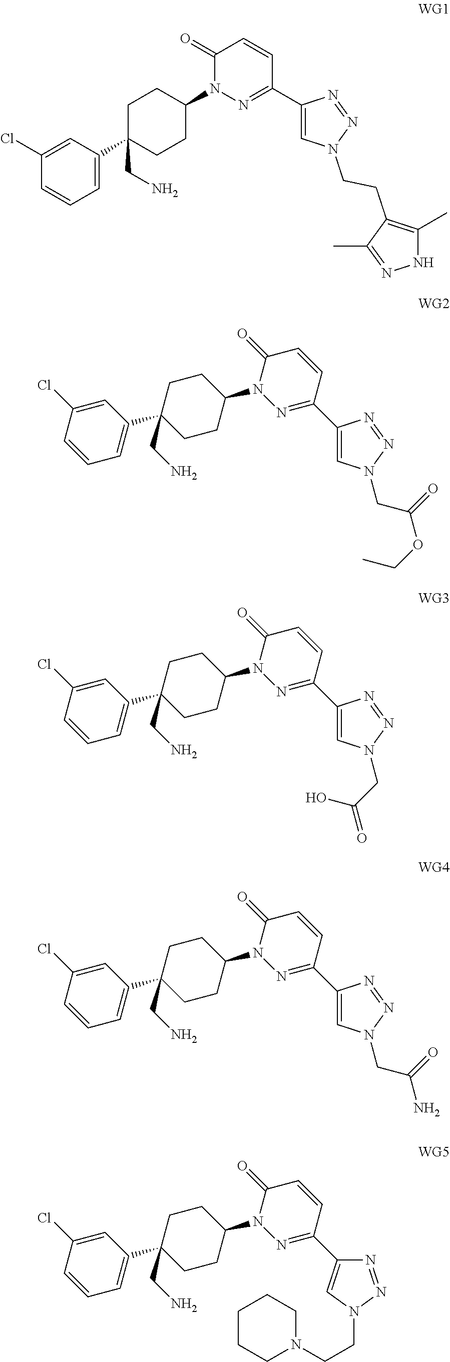 [SMILES: Cc1n[nH]c(C)c1CCn1cc(-c2ccc(=O)n([C@H]3CC[C@](CN)(c4cccc(Cl)c4)CC3)n2)nn1 ; CCOC(=O)Cn1cc(-c2ccc(=O)n([C@H]3CC[C@](CN)(c4cccc(Cl)c4)CC3)n2)nn1 ; NC[C@]1(c2cccc(Cl)c2)CC[C@H](n2nc(-c3cn(CC(=O)O)nn3)ccc2=O)CC1 ; NC[C@]1(c2cccc(Cl)c2)CC[C@H](n2nc(-c3cn(CC(N)=O)nn3)ccc2=O)CC1 ; NC[C@]1(c2cccc(Cl)c2)CC[C@H](n2nc(-c3cn(CCN4CCCCC4)nn3)ccc2=O)CC1]

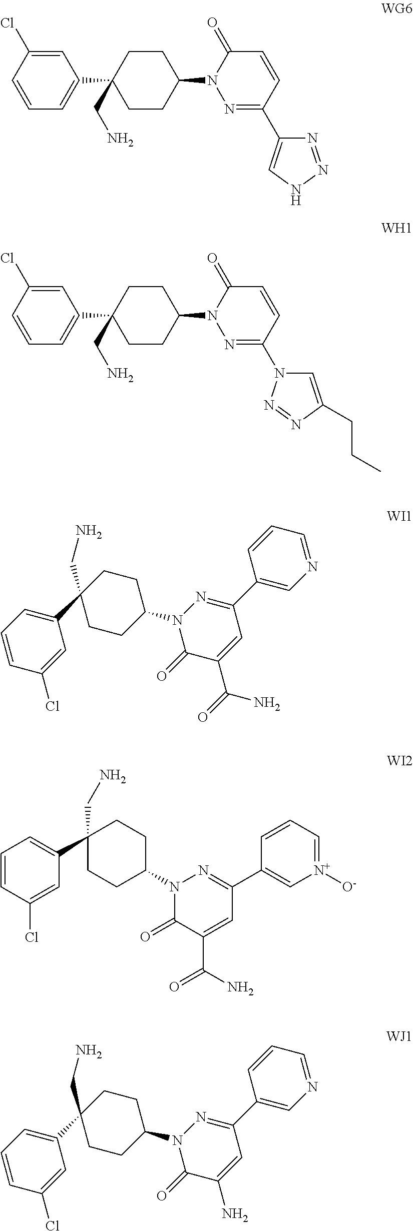 [SMILES: NC[C@]1(c2cccc(Cl)c2)CC[C@H](n2nc(-c3c[nH]nn3)ccc2=O)CC1 ; CCCc1cn(-c2ccc(=O)n([C@H]3CC[C@](CN)(c4cccc(Cl)c4)CC3)n2)nn1 ; NC[C@]1(c2cccc(Cl)c2)CC[C@H](n2nc(-c3cccnc3)cc(C(N)=O)c2=O)CC1 ; NC[C@]1(c2cccc(Cl)c2)CC[C@H](n2nc(-c3ccc[n+]([O-])c3)cc(C(N)=O)c2=O)CC1 ; NC[C@]1(c2cccc(Cl)c2)CC[C@H](n2nc(-c3cccnc3)cc(N)c2=O)CC1]

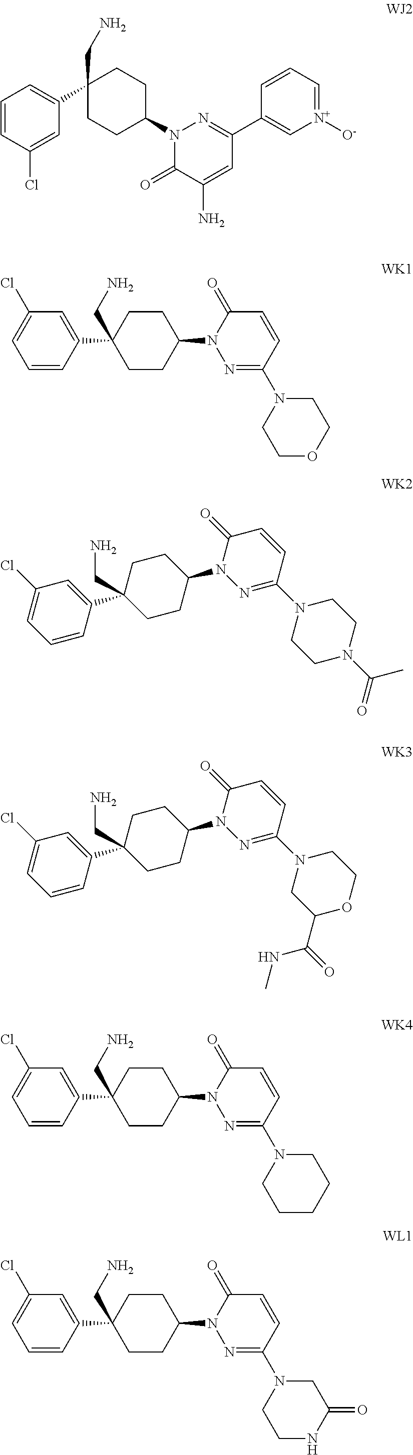 [SMILES: NC[C@]1(c2cccc(Cl)c2)CC[C@H](n2nc(-c3ccc[n+]([O-])c3)cc(N)c2=O)CC1 ; NC[C@]1(c2cccc(Cl)c2)CC[C@H](n2nc(N3CCOCC3)ccc2=O)CC1 ; CC(=O)N1CCN(c2ccc(=O)n([C@H]3CC[C@](CN)(c4cccc(Cl)c4)CC3)n2)CC1 ; CNC(=O)C1CN(c2ccc(=O)n([C@H]3CC[C@](CN)(c4cccc(Cl)c4)CC3)n2)CCO1 ; NC[C@]1(c2cccc(Cl)c2)CC[C@H](n2nc(N3CCCCC3)ccc2=O)CC1 ; NC[C@]1(c2cccc(Cl)c2)CC[C@H](n2nc(N3CCNC(=O)C3)ccc2=O)CC1]

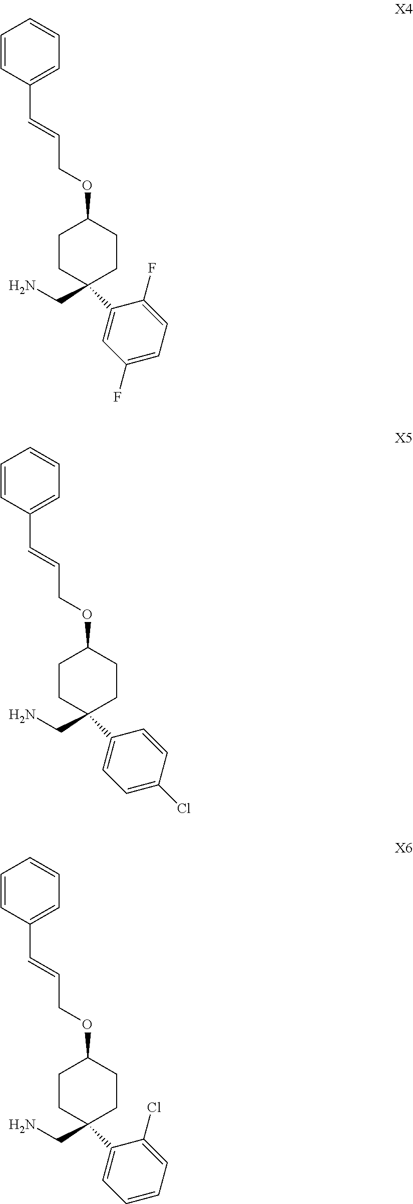 [SMILES: NC[C@]1(c2cc(F)ccc2F)CC[C@H](OC/C=C/c2ccccc2)CC1 ; NC[C@]1(c2ccc(Cl)cc2)CC[C@H](OC/C=C/c2ccccc2)CC1 ; NC[C@]1(c2ccccc2Cl)CC[C@H](OC/C=C/c2ccccc2)CC1]

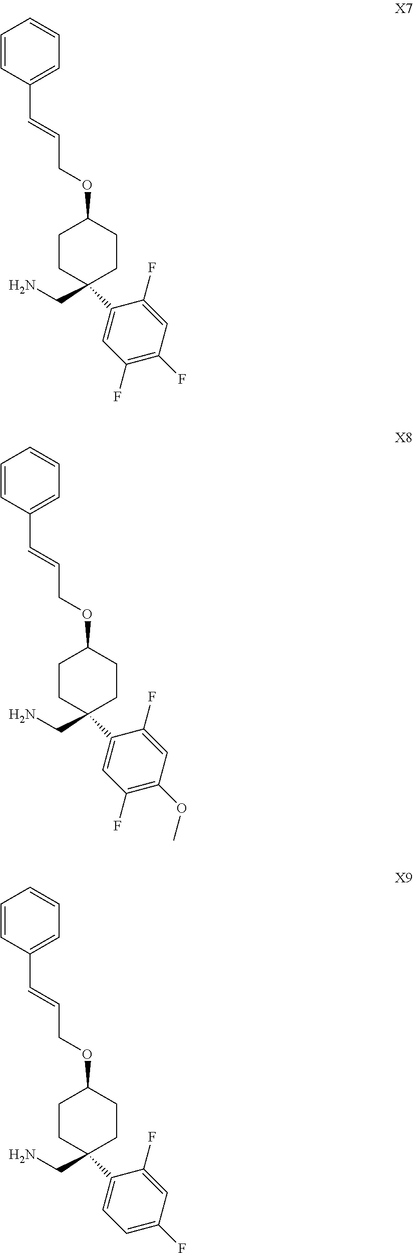 [SMILES: NC[C@]1(c2cc(F)c(F)cc2F)CC[C@H](OC/C=C/c2ccccc2)CC1 ; COc1cc(F)c([C@]2(CN)CC[C@@H](OC/C=C/c3ccccc3)CC2)cc1F ; NC[C@]1(c2ccc(F)cc2F)CC[C@H](OC/C=C/c2ccccc2)CC1]

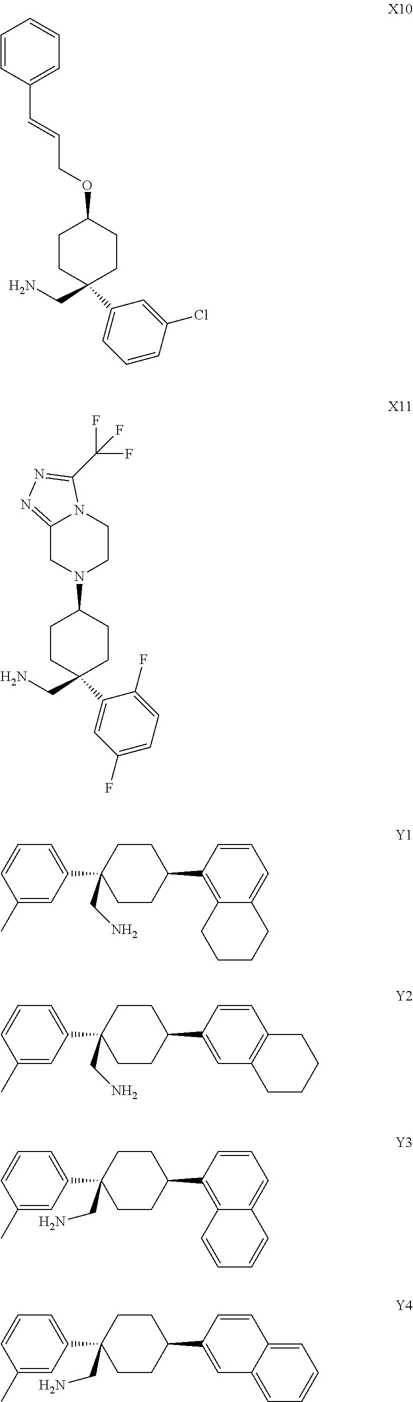

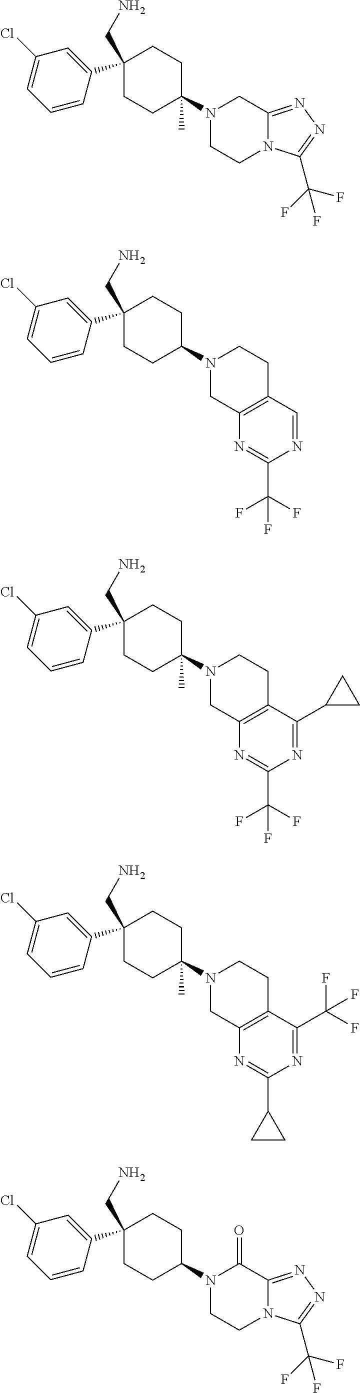 [SMILES: C[C@]1(N2CCn3c(nnc3C(F)(F)F)C2)CC[C@](CN)(c2cccc(Cl)c2)CC1 ; NC[C@]1(c2cccc(Cl)c2)CC[C@H](N2CCc3cnc(C(F)(F)F)nc3C2)CC1 ; C[C@]1(N2CCc3c(nc(C(F)(F)F)nc3C3CC3)C2)CC[C@](CN)(c2cccc(Cl)c2)CC1 ; C[C@]1(N2CCc3c(nc(C4CC4)nc3C(F)(F)F)C2)CC[C@](CN)(c2cccc(Cl)c2)CC1 ; NC[C@]1(c2cccc(Cl)c2)CC[C@H](N2CCn3c(nnc3C(F)(F)F)C2=O)CC1]

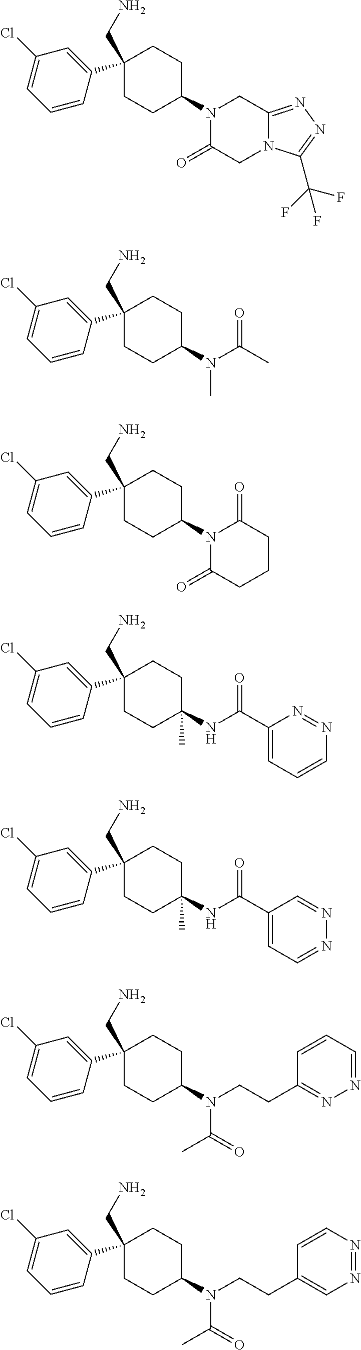

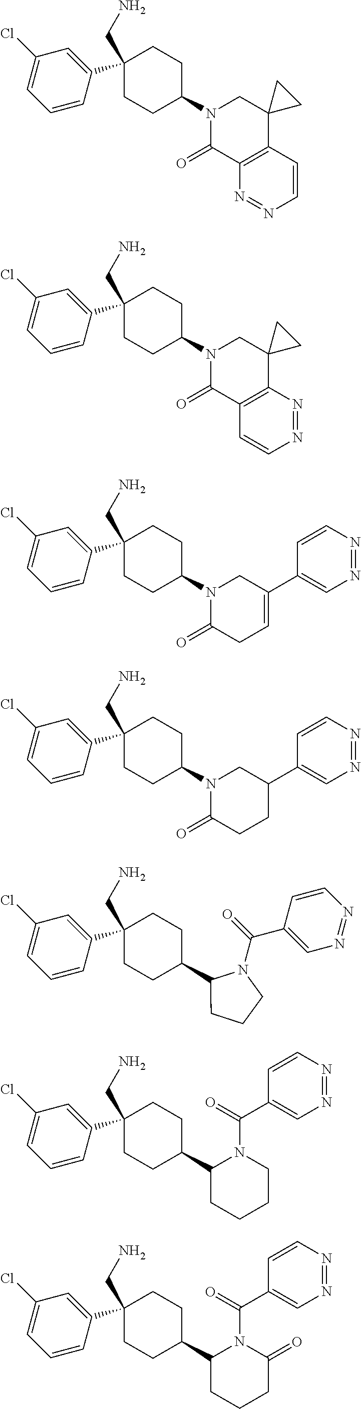

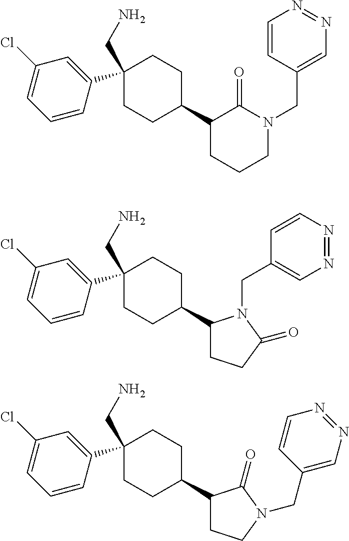 [SMILES: NC[C@]1(c2cccc(Cl)c2)CC[C@H](C2CCCN(Cc3ccnnc3)C2=O)CC1 ; NC[C@]1(c2cccc(Cl)c2)CC[C@H](C2CCC(=O)N2Cc2ccnnc2)CC1 ; NC[C@]1(c2cccc(Cl)c2)CC[C@H](C2CCN(Cc3ccnnc3)C2=O)CC1]

Compounds of the invention may be in the form of pharmaceutically acceptable salts. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., US, 1985, p. 1418, the disclosure of which is hereby incorporated by reference; see also Stahl et al, Eds, “Handbook of Pharmaceutical Salts Properties Selection and Use”, Verlag Helvetica Chimica Acta and Wiley-VCH, 2002.

The disclosure thus includes pharmaceutically-acceptable salts of the disclosed compounds wherein the parent compound is modified by making acid or base salts thereof. for example the conventional non-toxic salts or the quaternary ammonium salts which are formed, e.g. from inorganic or organic acids or bases. Examples of such acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine, lysine, and so forth. Also, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others.

The invention includes prodrugs for the active pharmaceutical species of the invention, for example in which one or more functional groups are protected or derivatised but can be converted in vivo to the functional group, as in the case of esters of carboxylic acids convertible in vivo to the free acid, or in the case of protected amines, to the free amino group. The term “prodrug,” as used herein, represents in particular compounds which are rapidly transformed in vivo to the parent compound, for example, by hydrolysis in blood. A thorough discussion is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987; H Bundgaard, ed, Design of Prodrugs, Elsevier, 1985; and Judkins, et al. Synthetic Communications, 26(23), 4351-4367 (1996), each of which is incorporated herein by reference.

Prodrugs therefore include drugs having a functional group which has been transformed into a reversible derivative thereof. Typically, such prodrugs are transformed to the active drug by hydrolysis. As examples may be mentioned the following:

| Functional Group | Reversible derivative |
|---|---|
| Carboxylic acid | Esters, including e.g. acyloxyalkyl esters, amides |
| Alcohol | Esters, including e.g. sulfates and phosphates as |
| | well as carboxylic acid esters |
| Amine | Amides, carbamates, imines, enamines, |
| Carbonyl (aldehyde, | Imines, oximes, acetals/ketals, enol esters, |
| ketone) | oxazolidines and thiazoxolidines |

Prodrugs also include compounds convertible to the active drug by an oxidative or reductive reaction. As examples may be mentioned:
Oxidative activation
   N- and O-dealkylation
   Oxidative deamination
   N-oxidation
   Epoxidation
Reductive activation
   Azo reduction
   Sulfoxide reduction
   Disulfide reduction
   Bioreductive alkylation
   Nitro reduction.

Also to be mentioned as metabolic activations of prodrugs are nucleotide activation, phosphorylation activation and decarboxylation activation. For additional information, see “The Organic Chemistry of Drug Design and Drug Action”, R B Silverman (particularly Chapter 8, pages 497 to 546), incorporated herein by reference.

The use of protecting groups is fully described in ‘Protective Groups in Organic Chemistry’, edited by J W F McOmie, Plenum Press (1973), and ‘Protective Groups in Organic Synthesis’, 2nd edition, T W Greene & P G M Wutz, Wiley-Interscience (1991).

Thus, it will be appreciated by those skilled in the art that, although protected derivatives of compounds of the disclosure may not possess pharmacological activity as such, they may be administered, for example parenterally or orally, and thereafter metabolised in the body to form compounds of the invention which are pharmacologically active. Such derivatives are therefore examples of “prodrugs”. All prodrugs of the described compounds are included within the scope of the disclosure.

Some groups mentioned herein (especially those containing heteroatoms and conjugated bonds) may exist in tautomeric forms and all these tautomers are included in the scope of the disclosure. More generally, many species may exist in equilibrium, as for example in the case of organic acids and their counterpart anions; a reference herein to a species accordingly includes reference to all equilibrium forms thereof.

The compounds of the disclosure may also contain one or more asymmetric carbon atoms and may therefore exhibit optical and/or diastereoisomerism. All diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation, or by derivatisation, for example with a homochiral acid followed by separation of the diastereomeric derivatives by conventional means (e.g. HPLC, chromatography over silica). All stereoisomers are included within the scope of the disclosure. Where a single enantiomer or diasteromer is disclosed, the disclosure also covers the other enantiomers or diastereomers, and also racemates; in this regard, particular reference is made to the specific compounds listed herein.

Geometric isomers may also exist in the compounds of the present disclosure. The present disclosure contemplates the various geometric isomers and mixtures thereof resulting from the arrangement of substituents around a carbon-carbon double bond and designates such isomers as of the Z or E configuration, wherein the term “Z” represents substituents on the same side of the carbon—carbon double bond and the term “E” represents substituents on opposite sides of the carbon—carbon double bond.

The disclosure therefore includes all variant forms of the defined compounds, for example any tautomer or any pharmaceutically acceptable salt, ester, acid or other variant of the defined compounds and their tautomers as well as substances which, upon administration, are capable of providing directly or indirectly a compound as defined above or providing a species which is capable of existing in equilibrium with such a compound.

### Synthesis

By way of illustration, a compound of the invention may be prepared according to any of the following general reaction schemes:

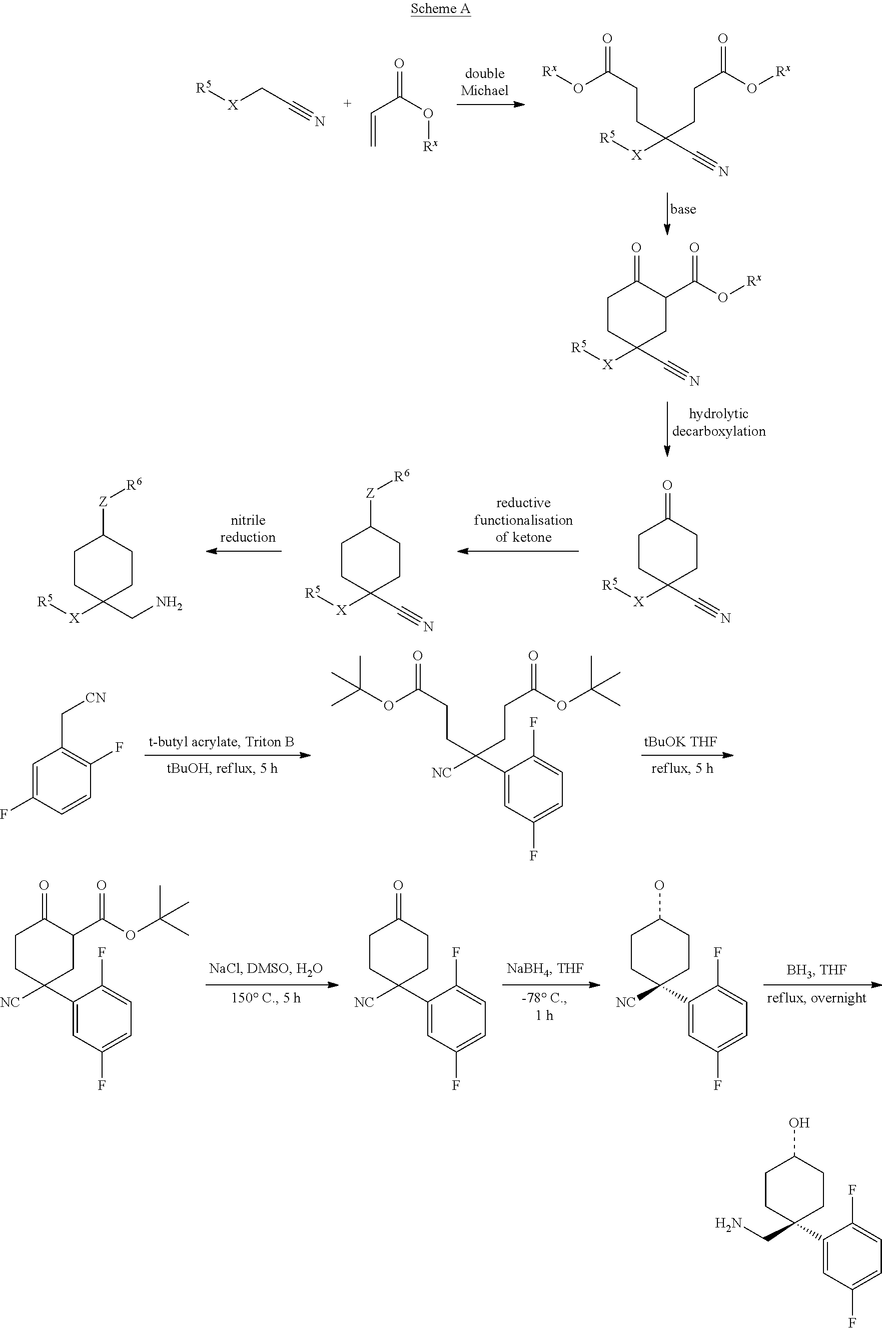

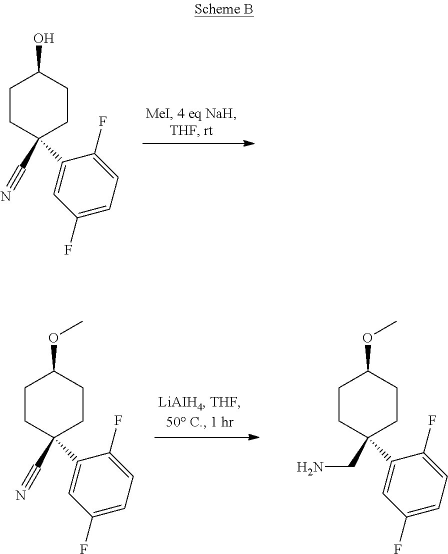 [SMILES: N#C[C@]1(c2cc(F)ccc2F)CC[C@H](O)CC1 ; CO[C@H]1CC[C@](C#N)(c2cc(F)ccc2F)CC1 ; CO[C@H]1CC[C@](CN)(c2cc(F)ccc2F)CC1]

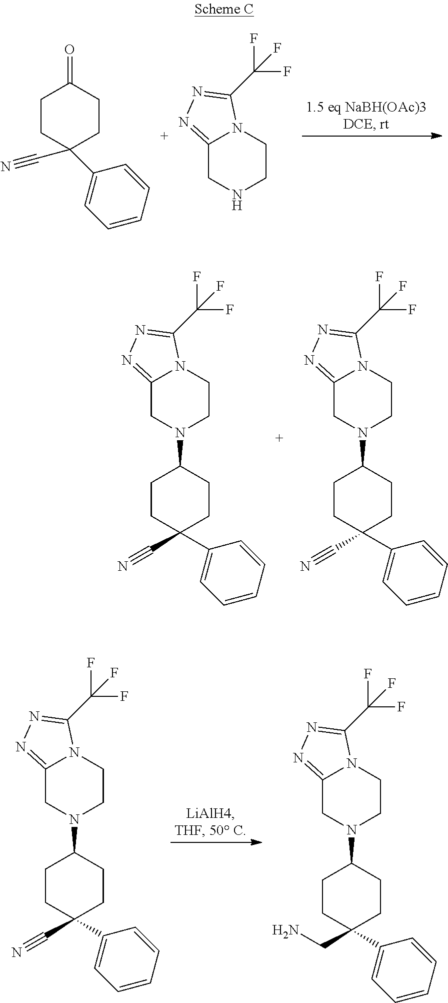

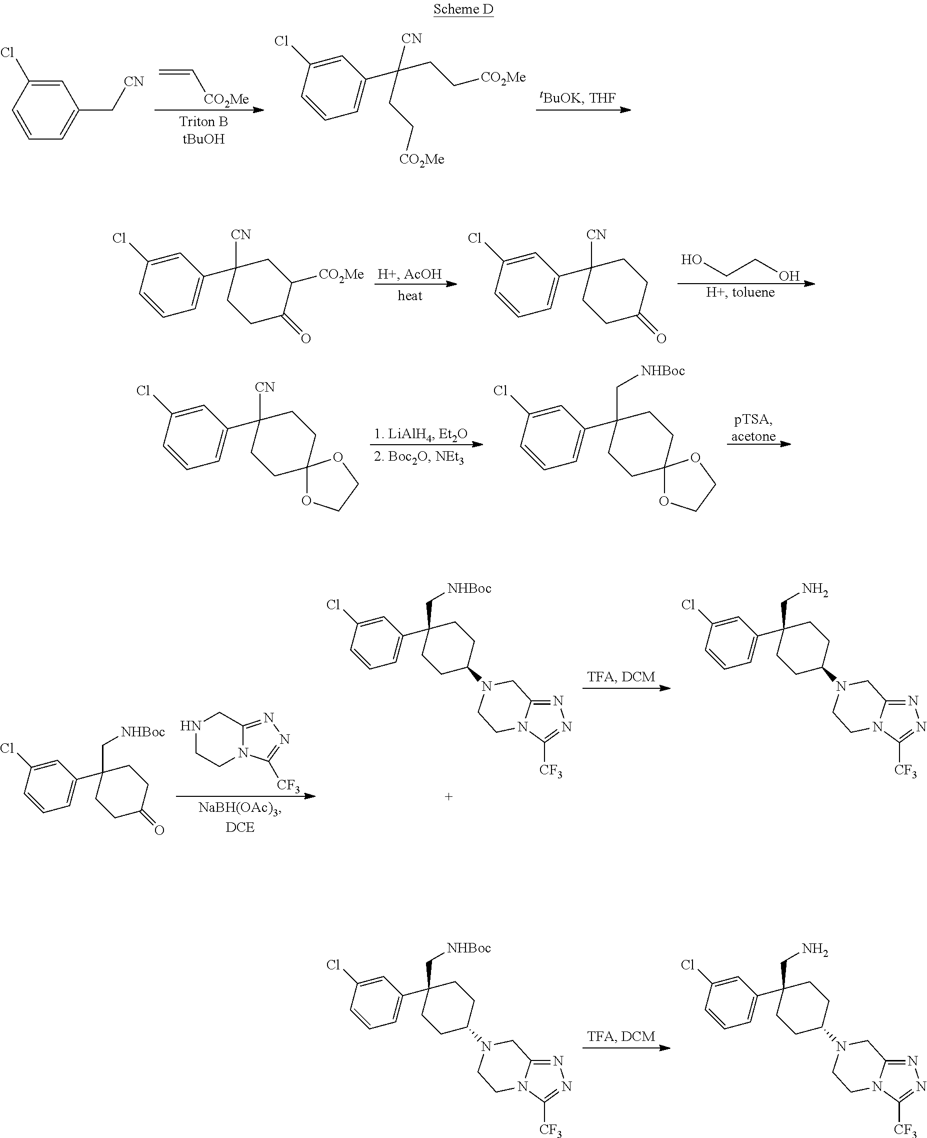

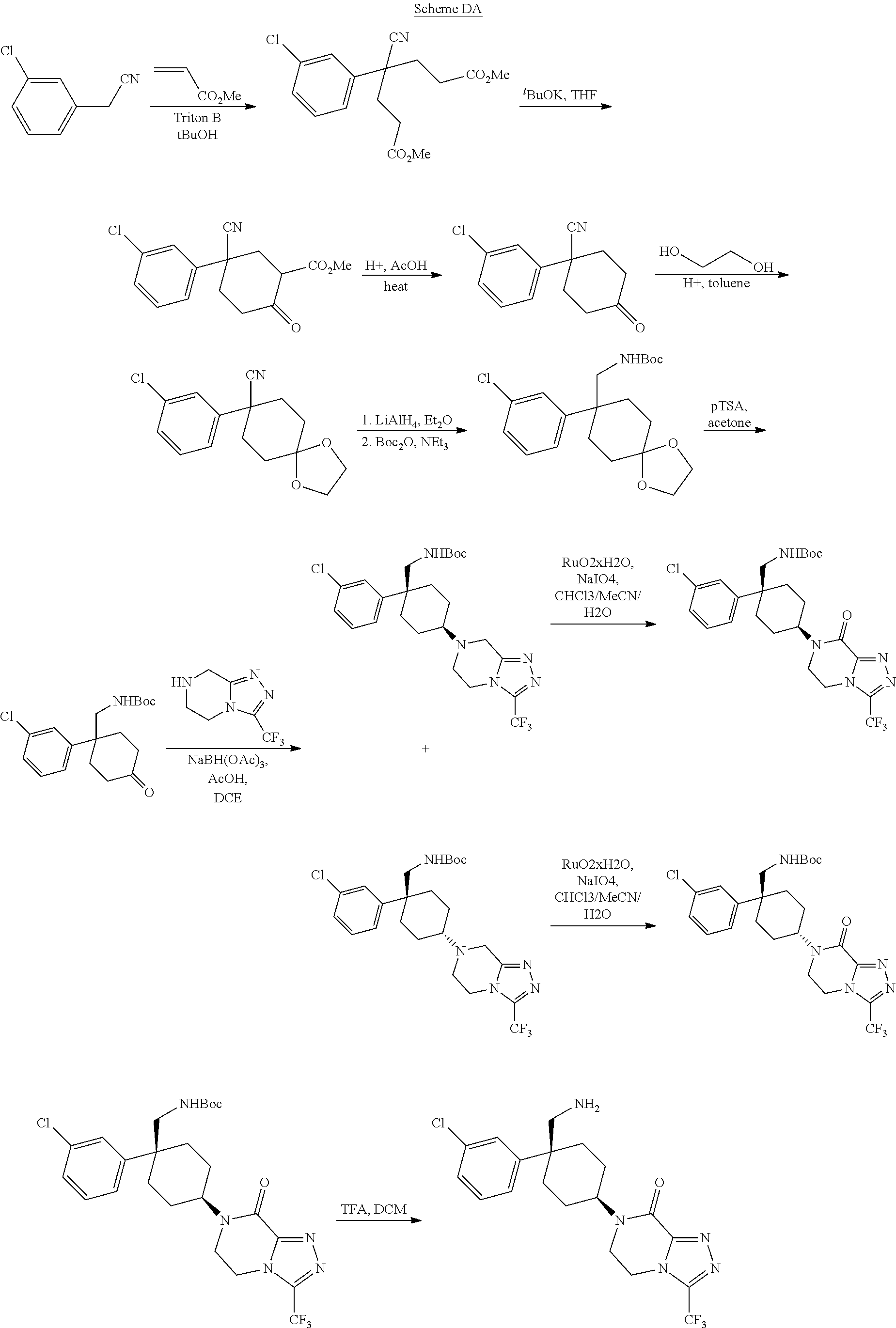

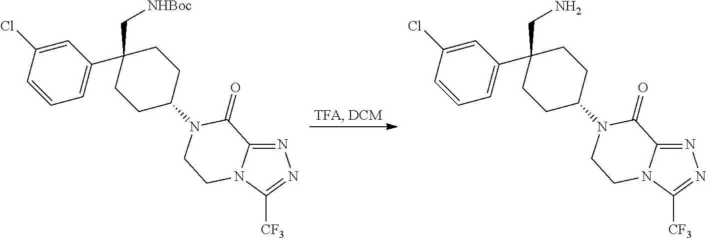 [SMILES: CC(C)(C)OC(=O)NC[C@]1(c2cccc(Cl)c2)CC[C@@H](N2CCn3c(nnc3C(F)(F)F)C2=O)CC1 ; NC[C@]1(c2cccc(Cl)c2)CC[C@@H](N2CCn3c(nnc3C(F)(F)F)C2=O)CC1]

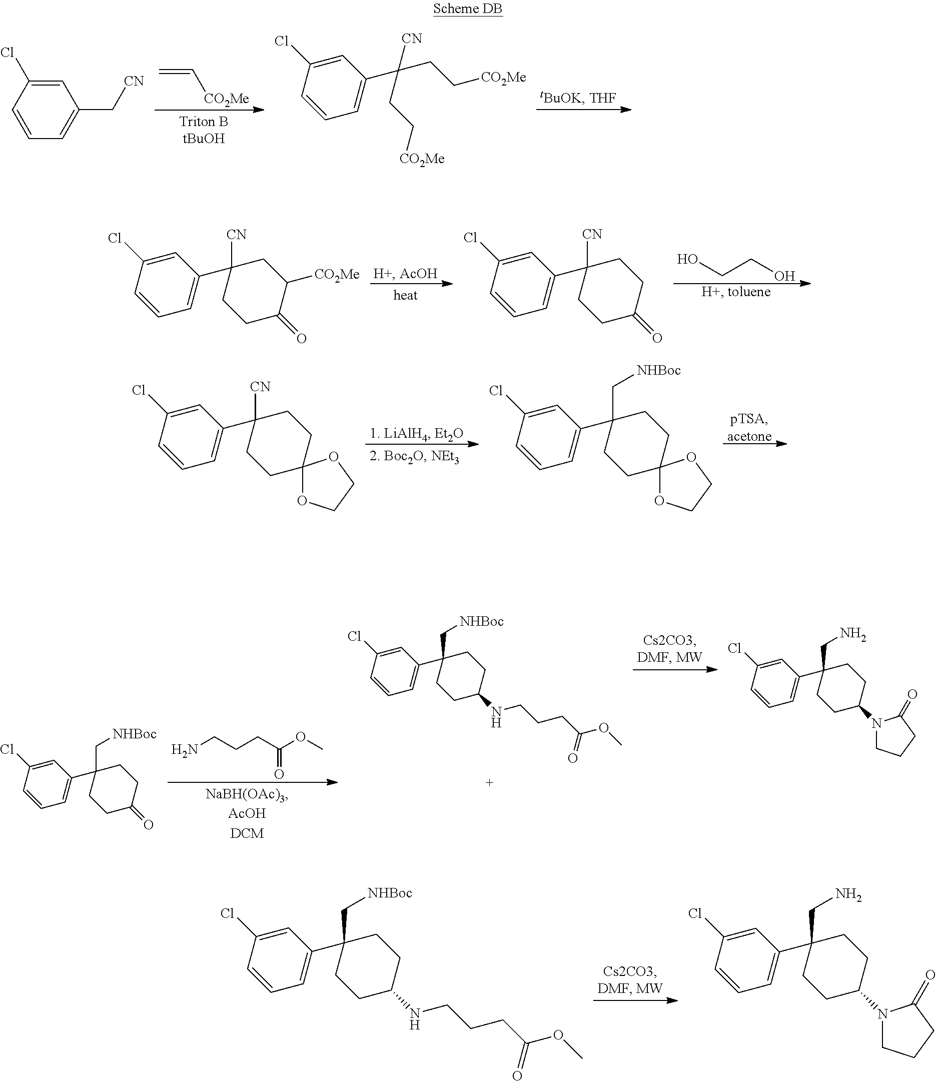

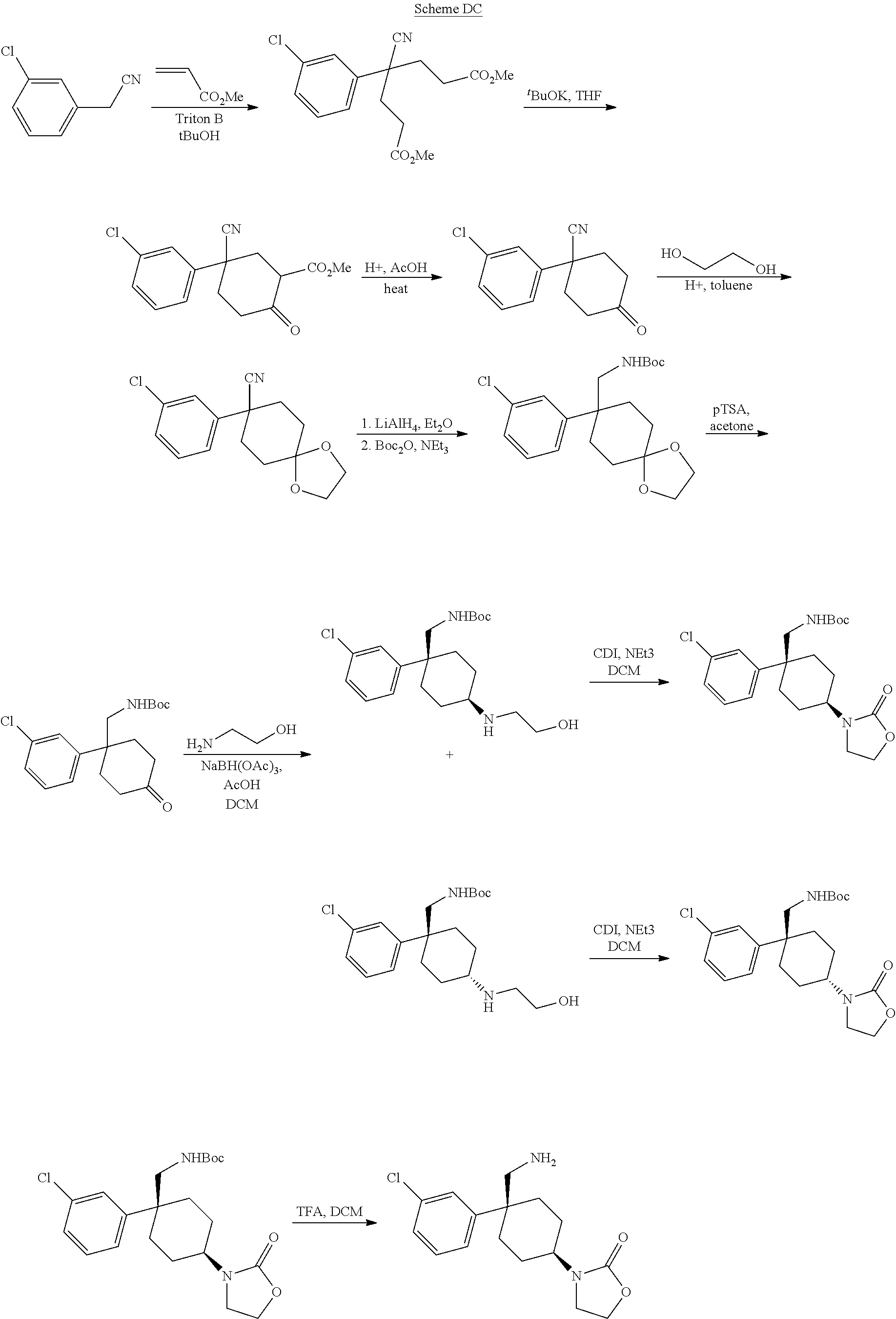

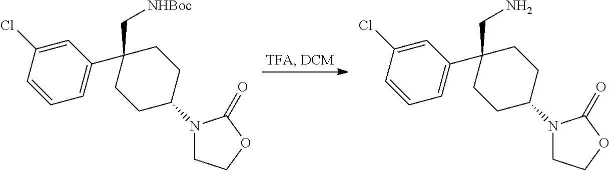 [SMILES: CC(C)(C)OC(=O)NC[C@]1(c2cccc(Cl)c2)CC[C@@H](N2CCOC2=O)CC1 ; NC[C@]1(c2cccc(Cl)c2)CC[C@@H](N2CCOC2=O)CC1]

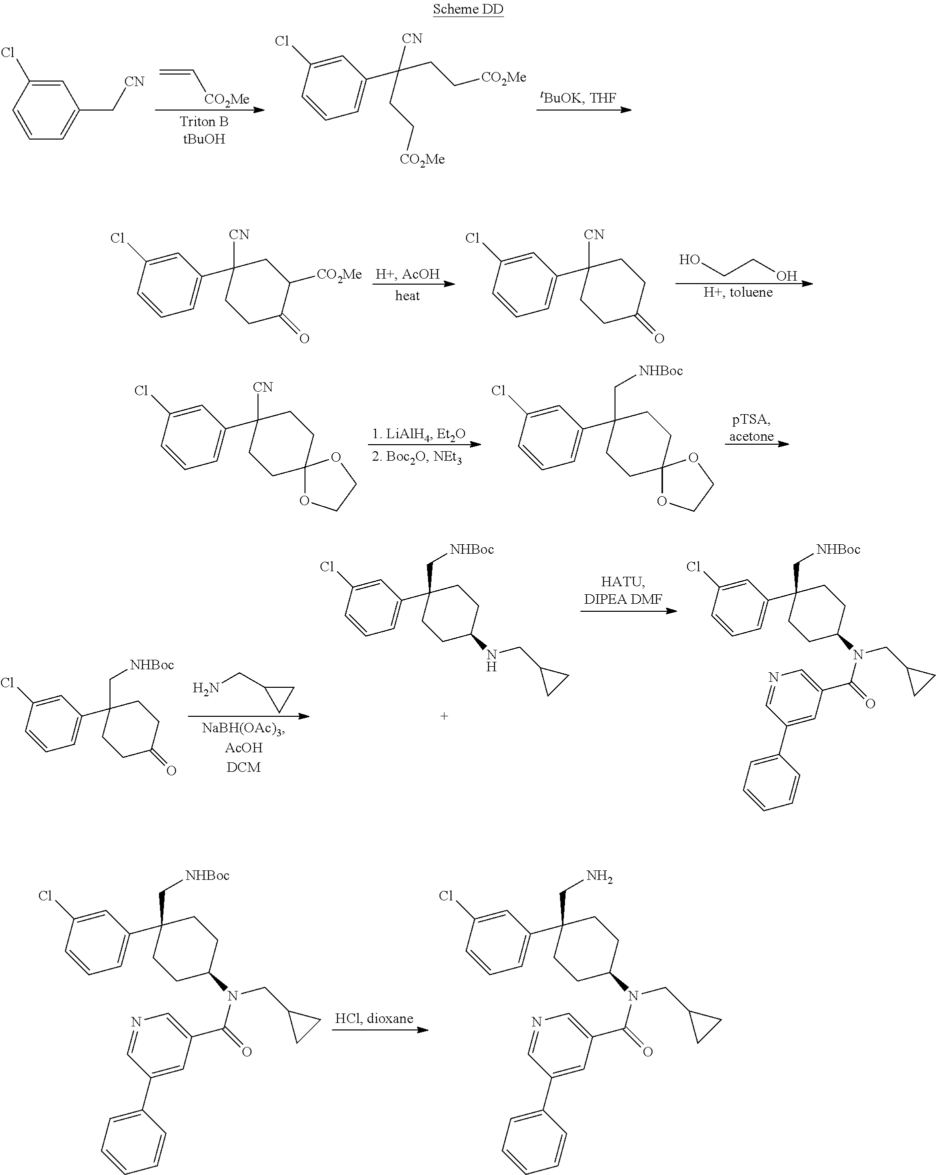

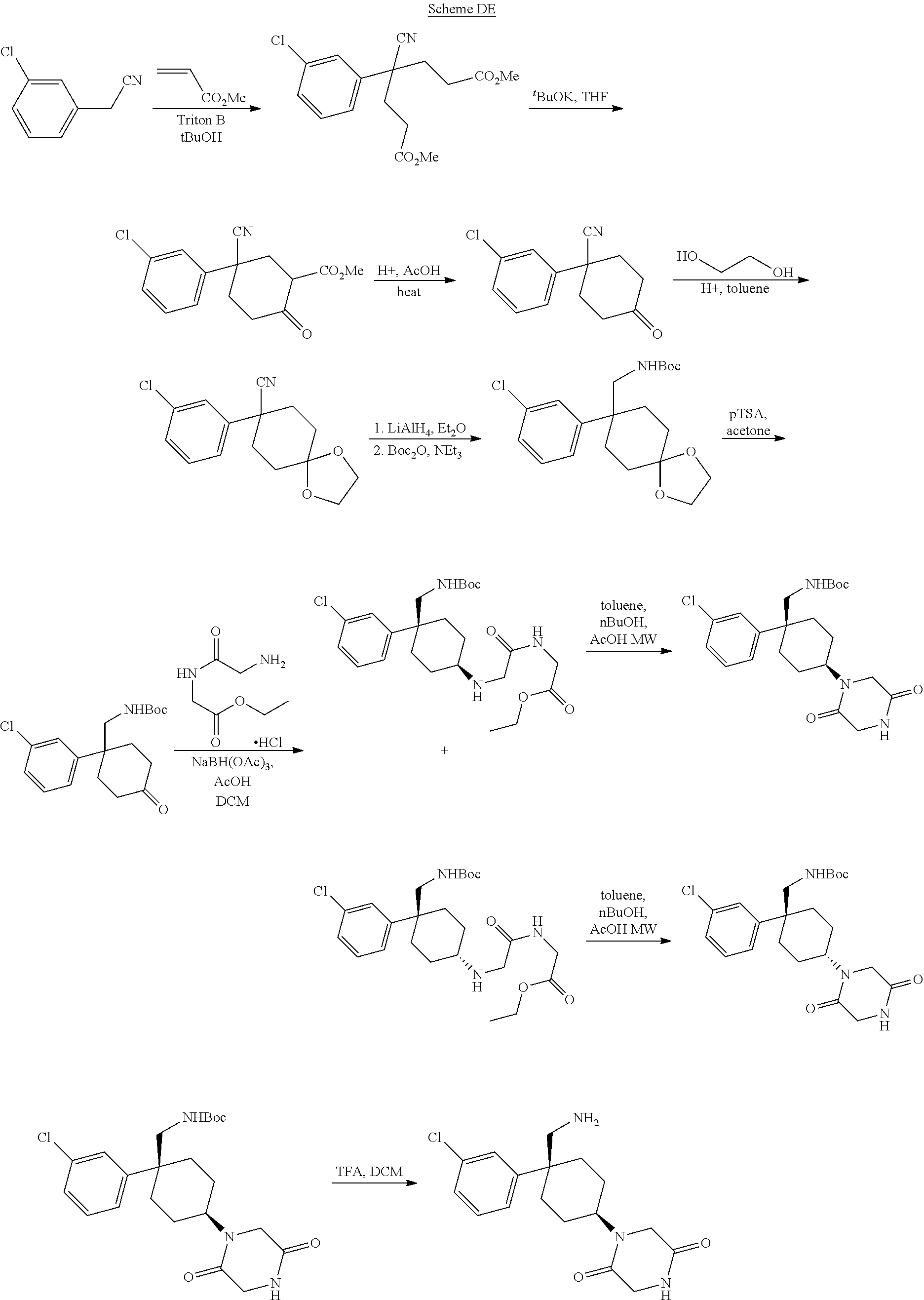

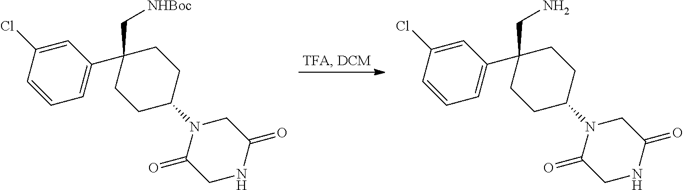 [SMILES: CC(C)(C)OC(=O)NC[C@]1(c2cccc(Cl)c2)CC[C@@H](N2CC(=O)NCC2=O)CC1 ; NC[C@]1(c2cccc(Cl)c2)CC[C@@H](N2CC(=O)NCC2=O)CC1]

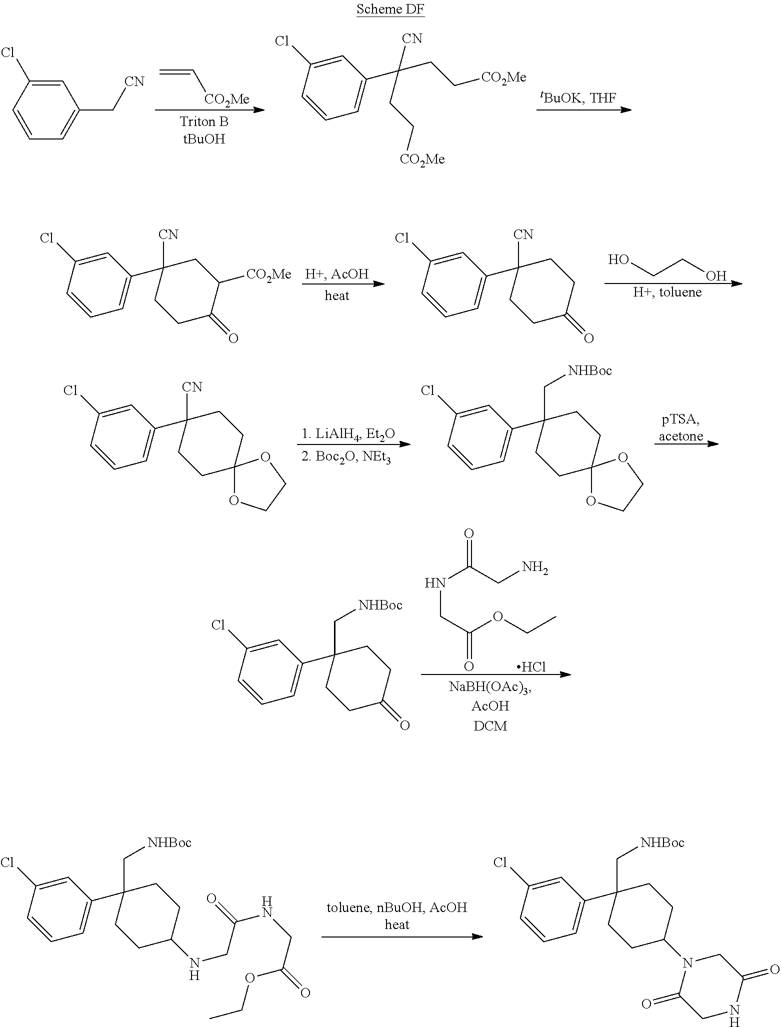

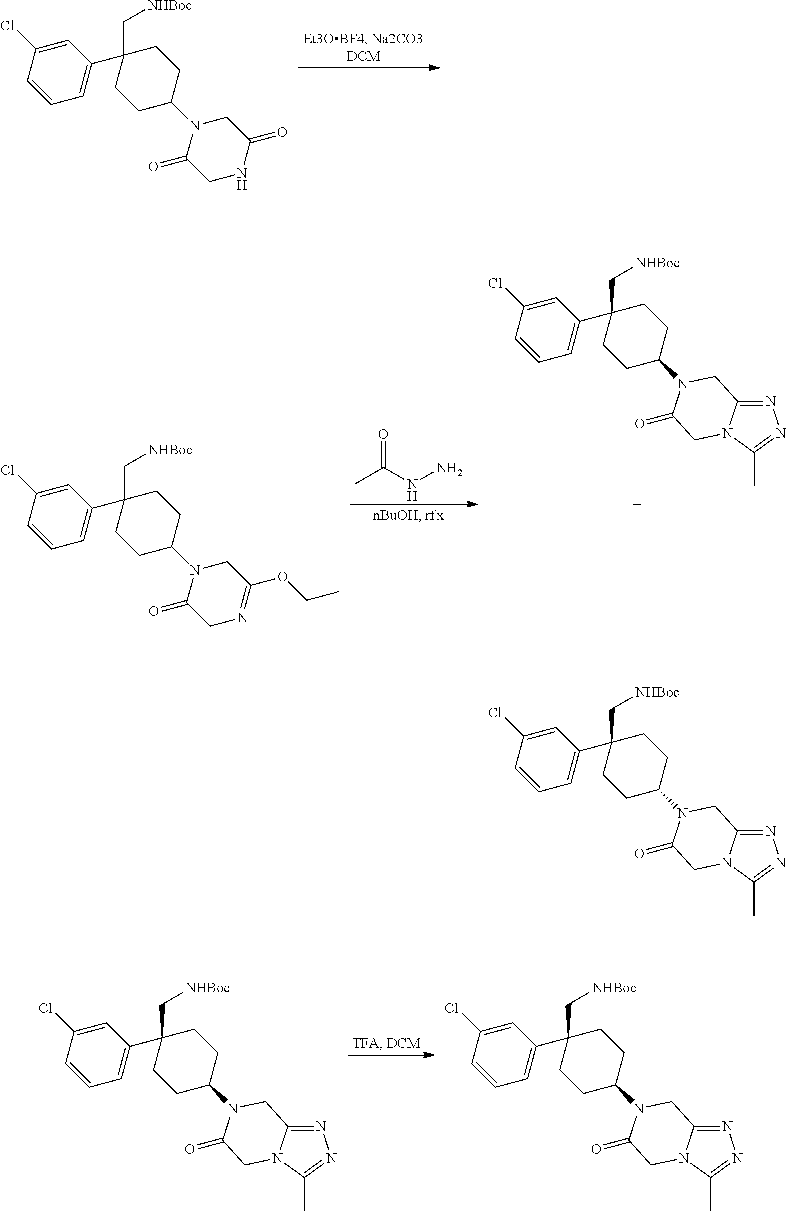

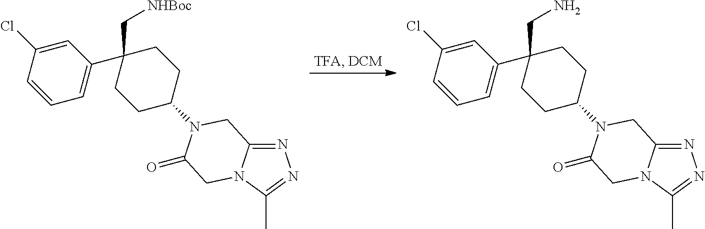 [SMILES: Cc1nnc2n1CC(=O)N([C@H]1CC[C@@](CNC(=O)OC(C)(C)C)(c3cccc(Cl)c3)CC1)C2 ; Cc1nnc2n1CC(=O)N([C@H]1CC[C@@](CN)(c3cccc(Cl)c3)CC1)C2]

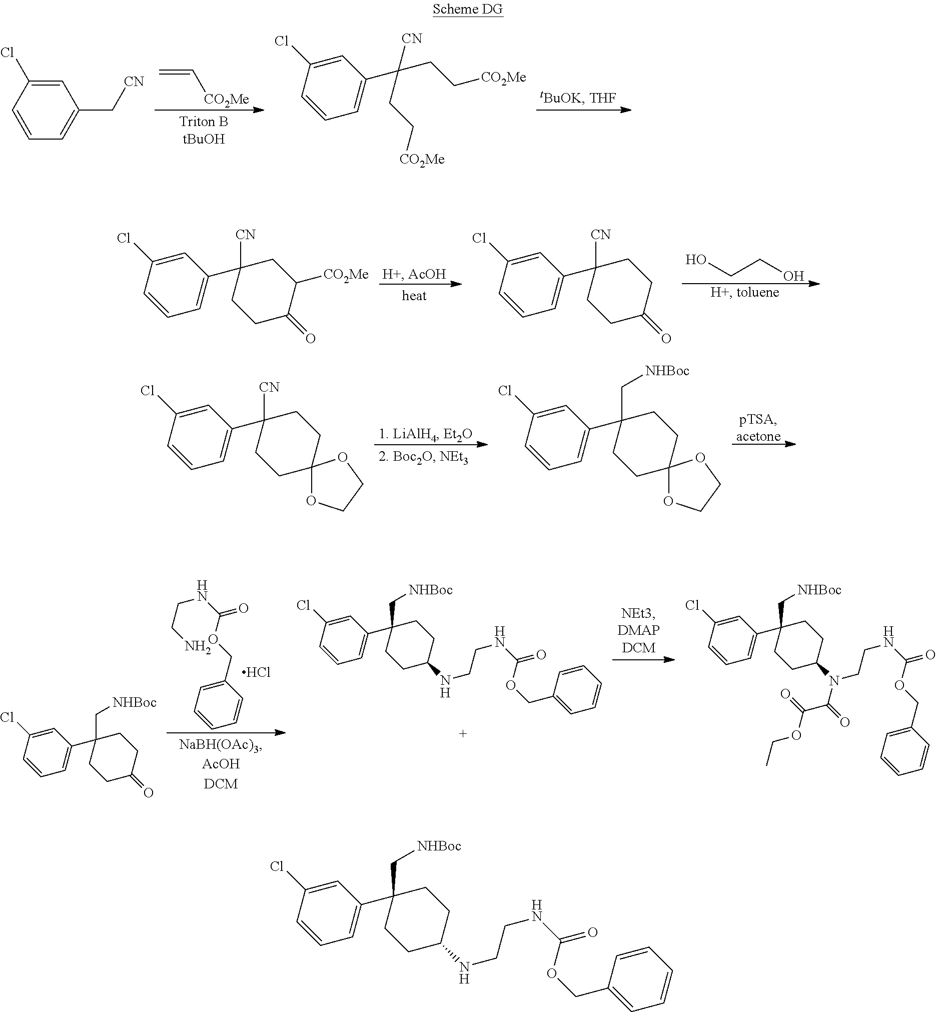

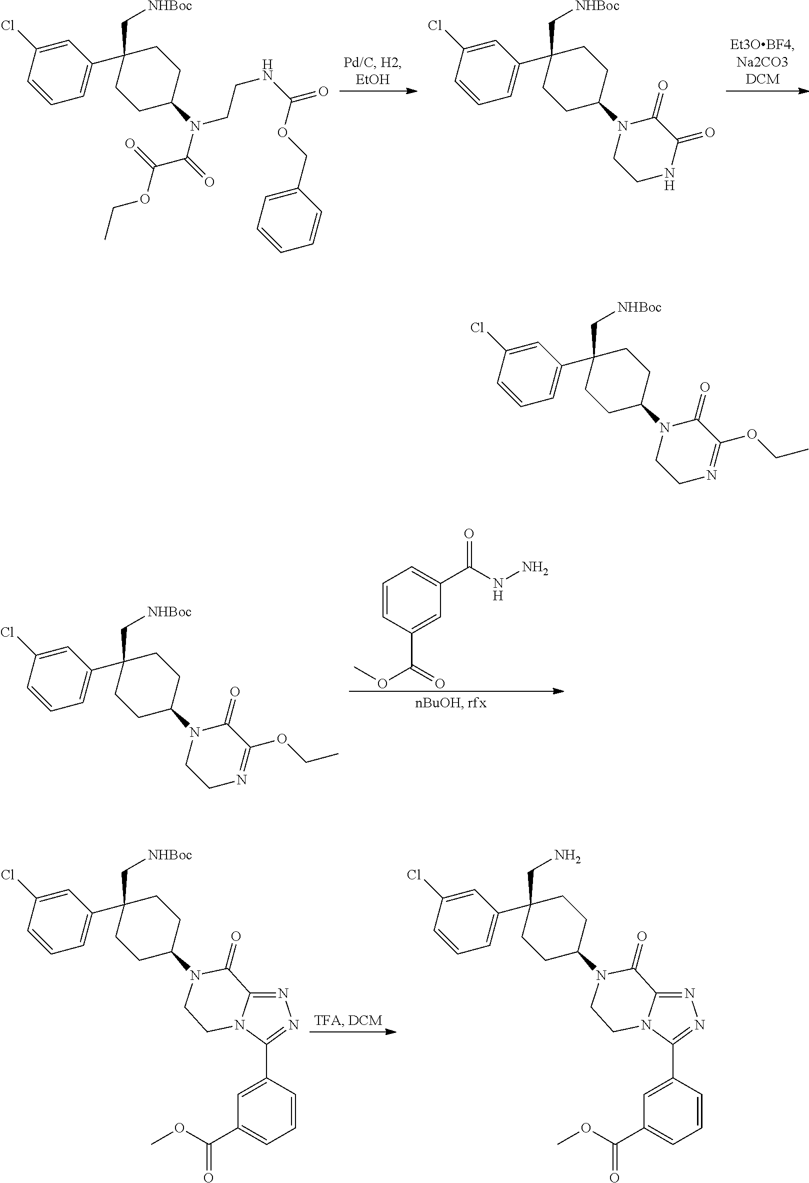

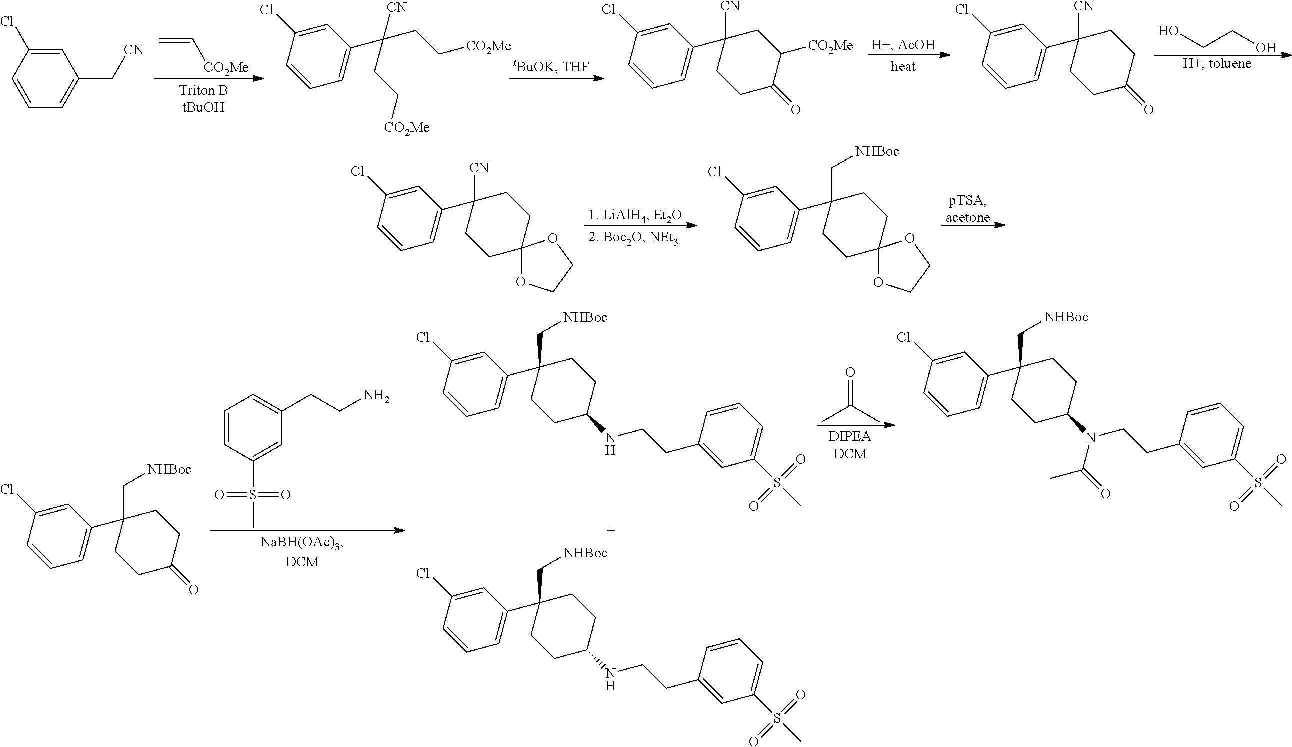

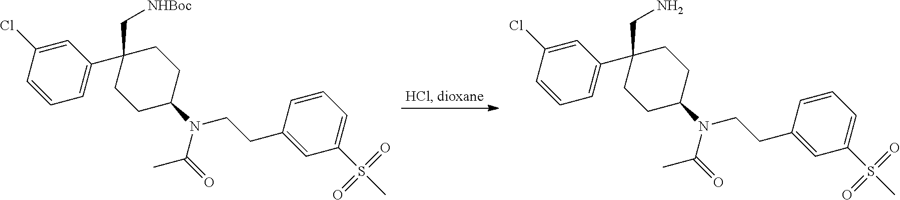 [SMILES: CC(=O)N(CCc1cccc(S(C)(=O)=O)c1)[C@H]1CC[C@](CNC(=O)OC(C)(C)C)(c2cccc(Cl)c2)CC1 ; CC(=O)N(CCc1cccc(S(C)(=O)=O)c1)[C@H]1CC[C@](CN)(c2cccc(Cl)c2)CC1]

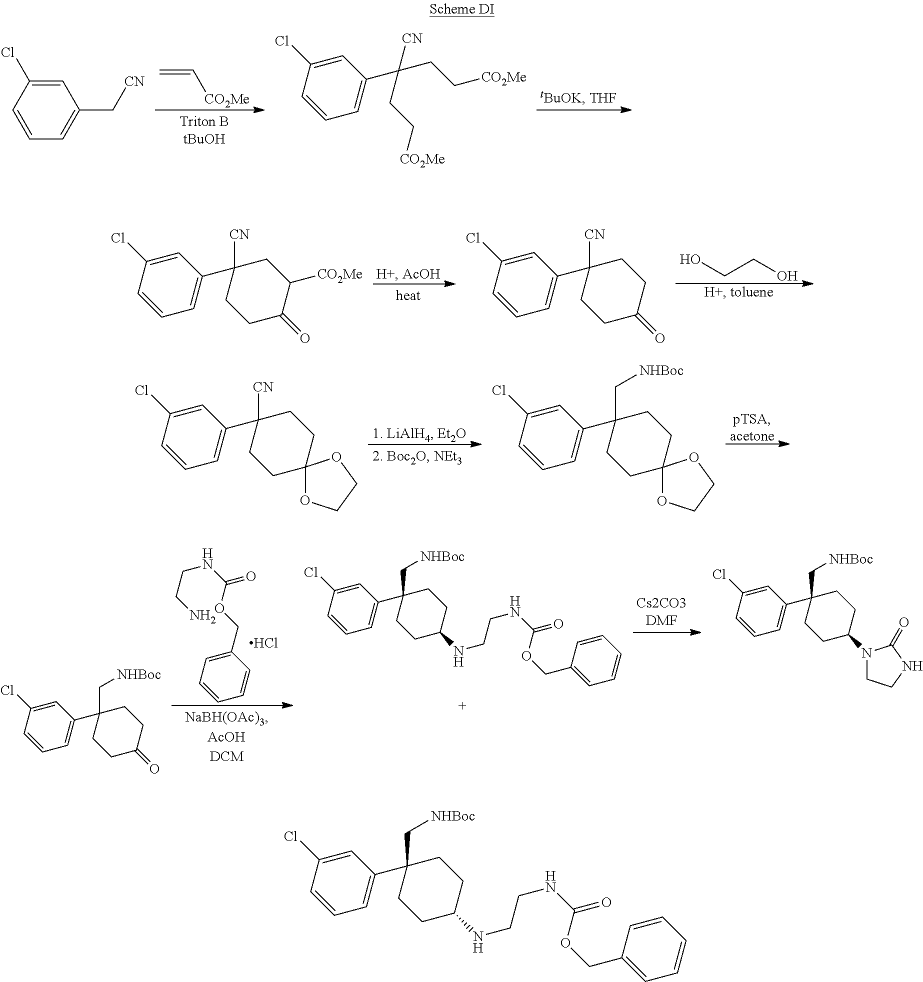

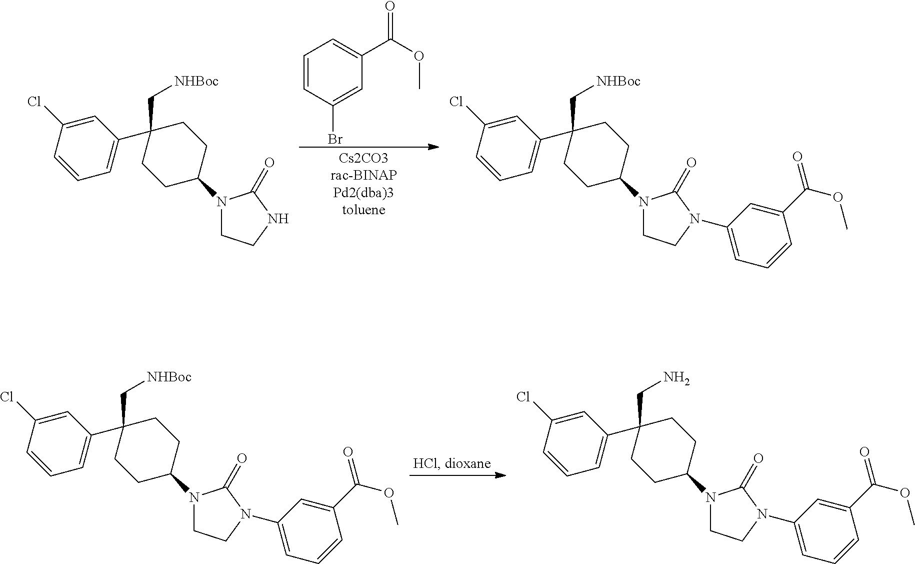

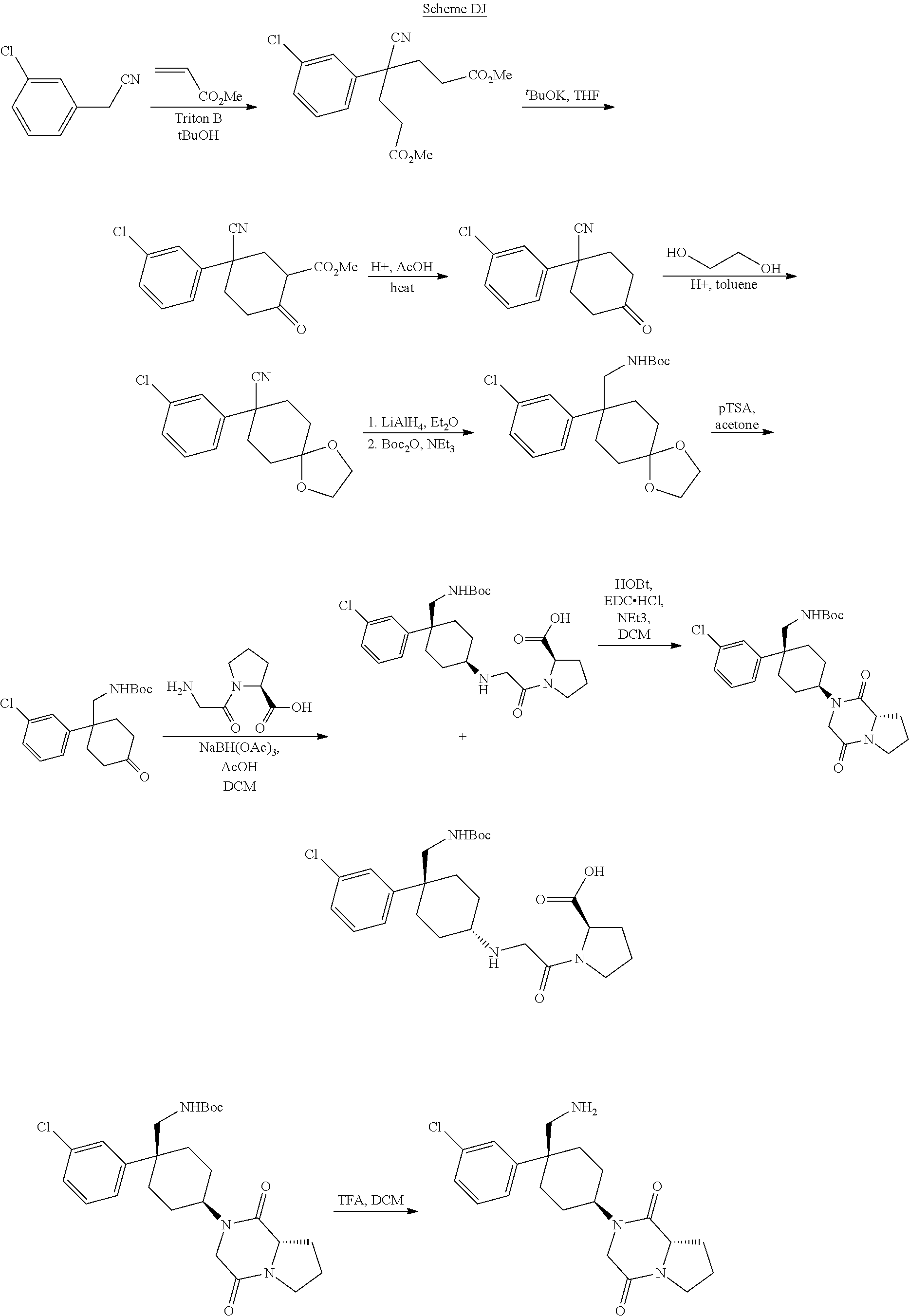

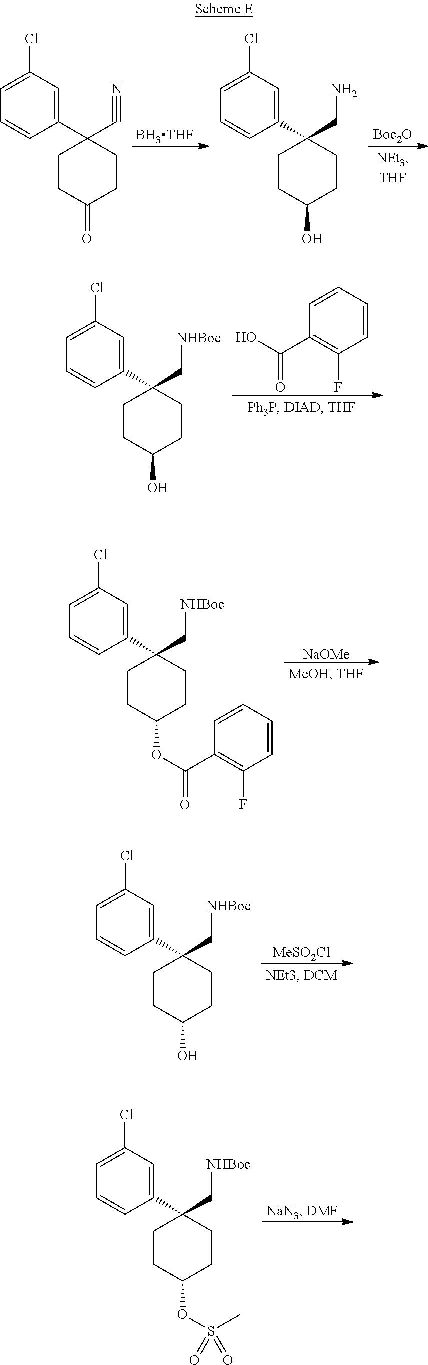

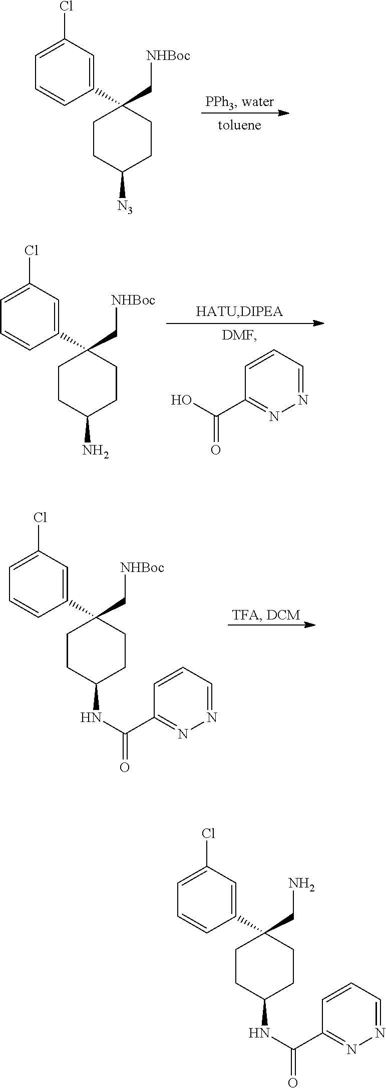

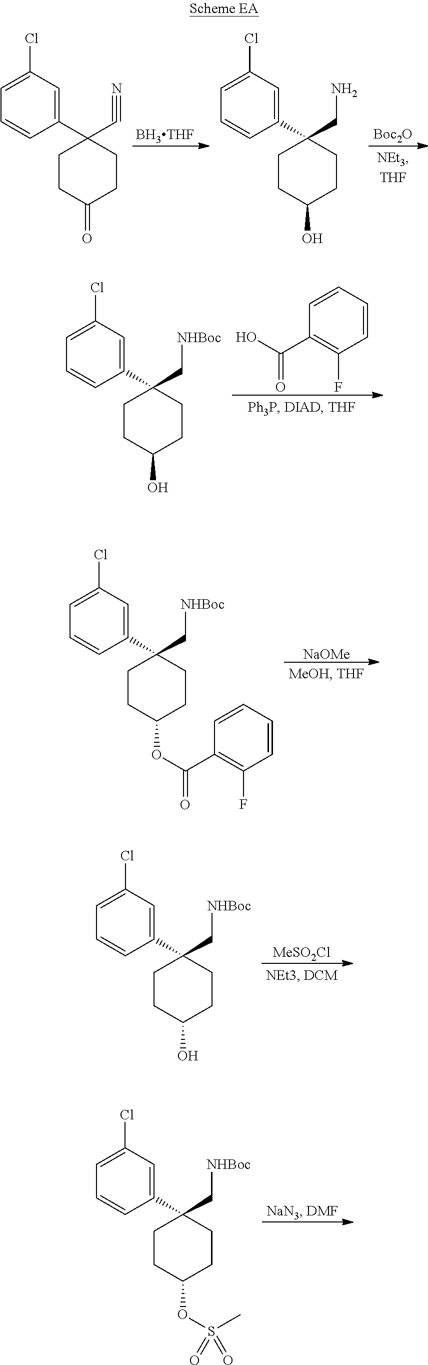

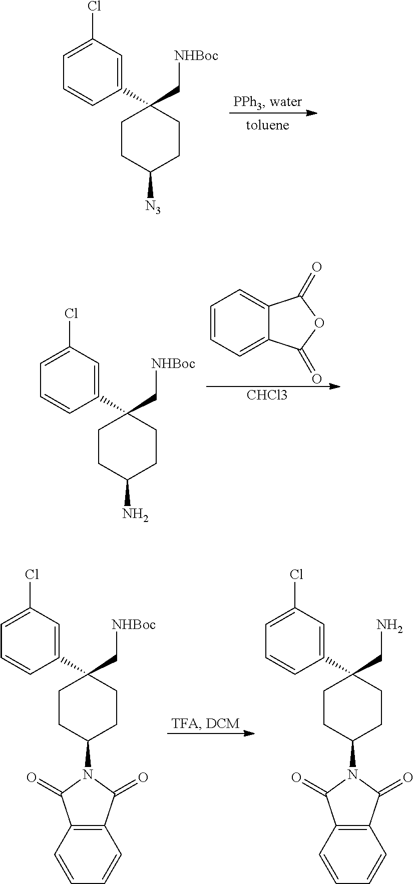

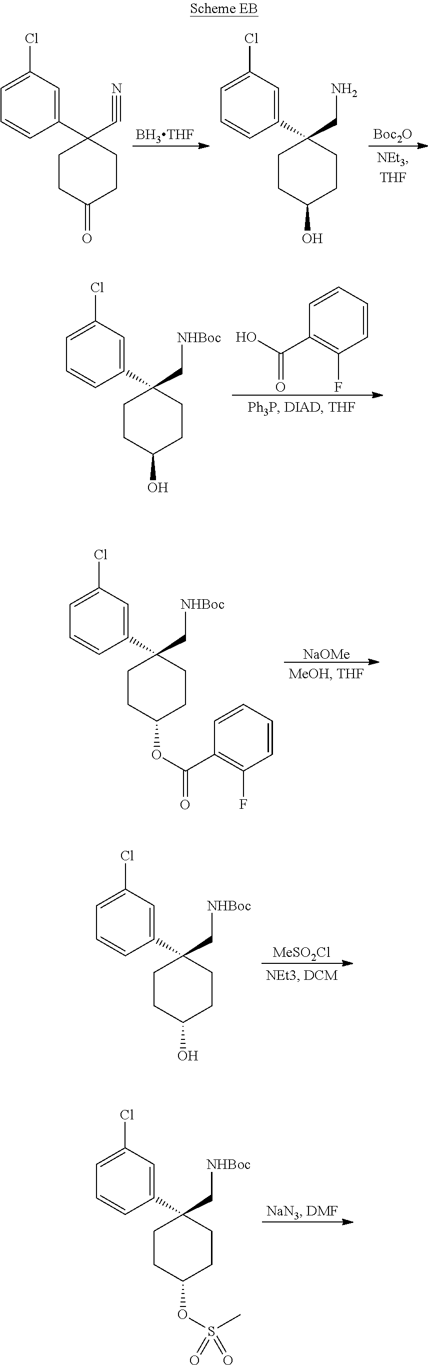

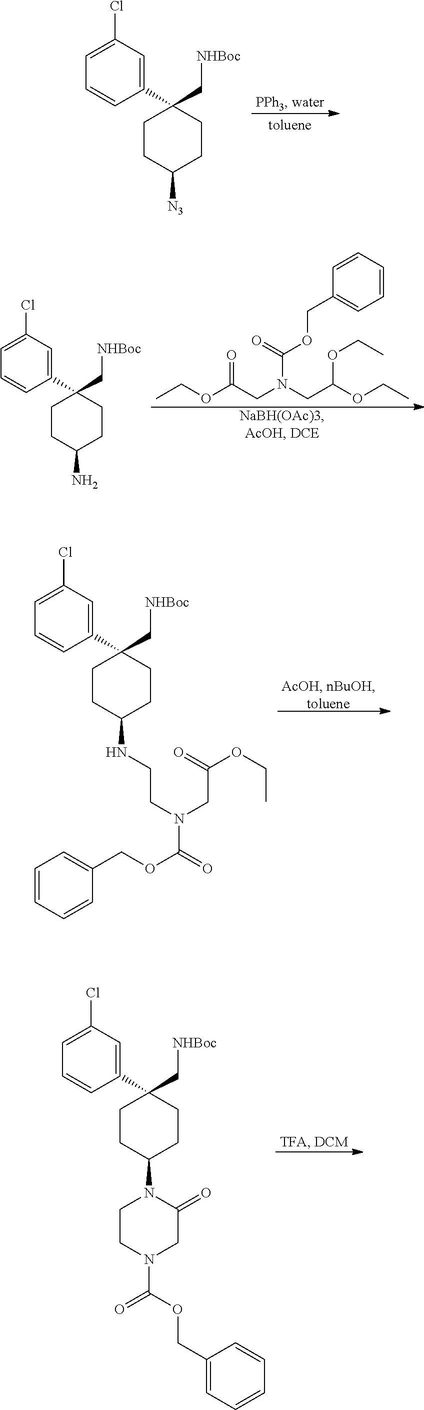

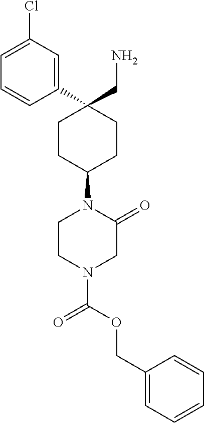 [SMILES: NC[C@]1(c2cccc(Cl)c2)CC[C@H](N2CCN(C(=O)OCc3ccccc3)CC2=O)CC1]

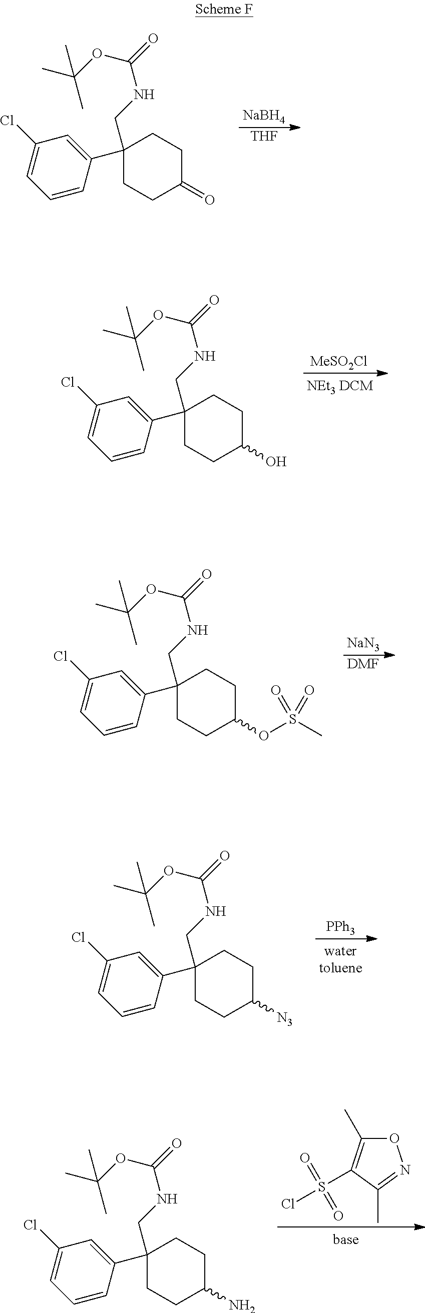

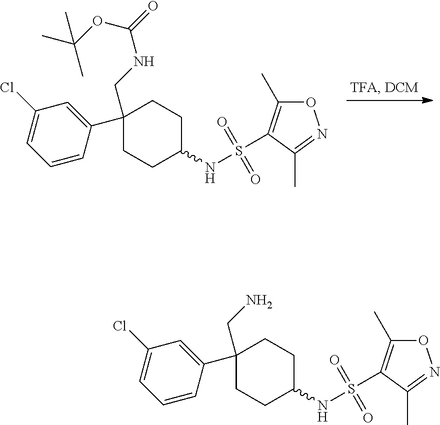 [SMILES: Cc1noc(C)c1S(=O)(=O)NC1CCC(CNC(=O)OC(C)(C)C)(c2cccc(Cl)c2)CC1 ; Cc1noc(C)c1S(=O)(=O)NC1CCC(CN)(c2cccc(Cl)c2)CC1]

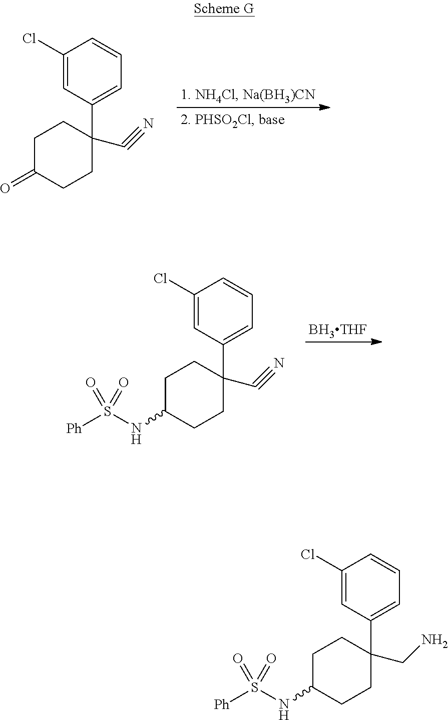 [SMILES: N#CC1(c2cccc(Cl)c2)CCC(=O)CC1 ; N#CC1(c2cccc(Cl)c2)CCC(NS(=O)(=O)c2ccccc2)CC1 ; NCC1(c2cccc(Cl)c2)CCC(NS(=O)(=O)c2ccccc2)CC1 ; "BH3·THF"]

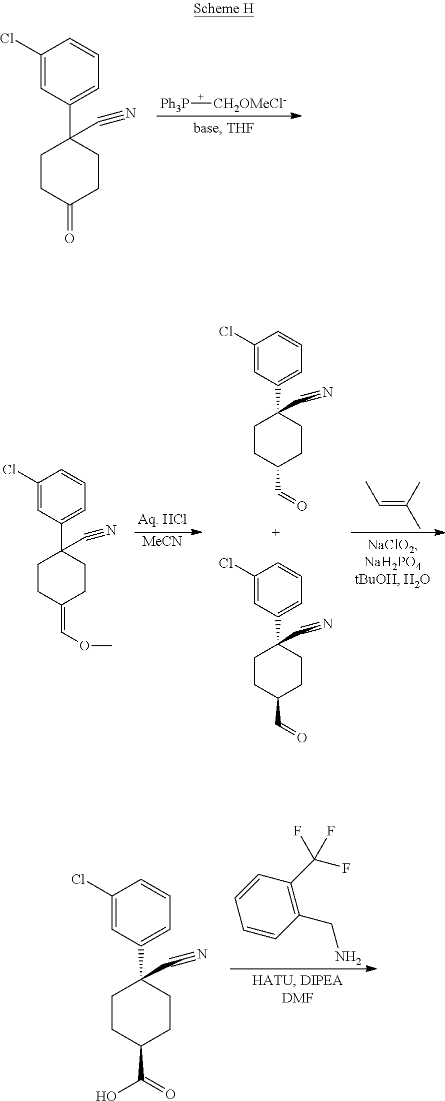

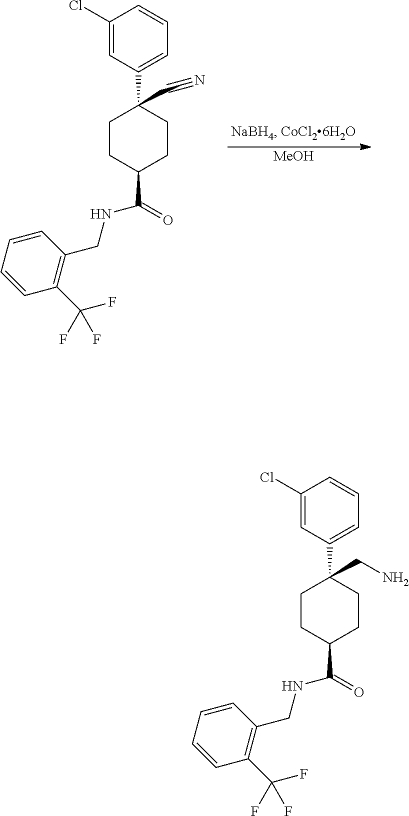 [SMILES: "MeOH" ; N#C[C@]1(c2cccc(Cl)c2)CC[C@H](C(=O)NCc2ccccc2C(F)(F)F)CC1 ; NC[C@]1(c2cccc(Cl)c2)CC[C@H](C(=O)NCc2ccccc2C(F)(F)F)CC1]

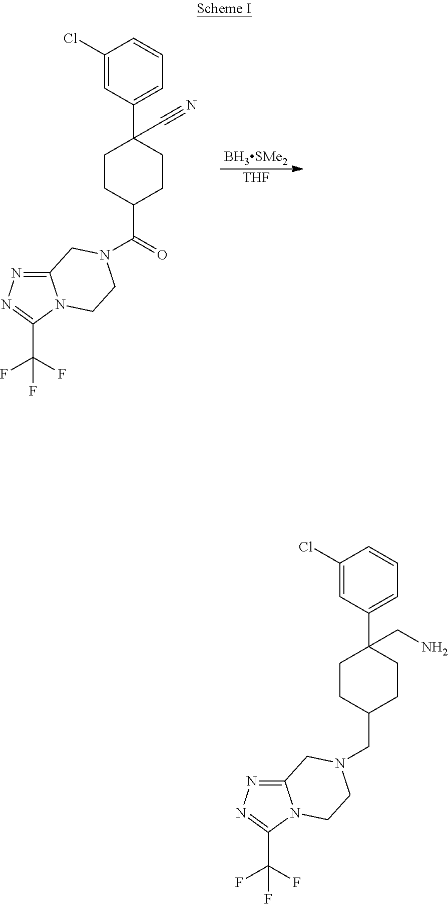 [SMILES: N#CC1(c2cccc(Cl)c2)CCC(C(=O)N2CCn3c(nnc3C(F)(F)F)C2)CC1 ; "BH3·SMe2" ; NCC1(c2cccc(Cl)c2)CCC(CN2CCn3c(nnc3C(F)(F)F)C2)CC1 ; "THF"]

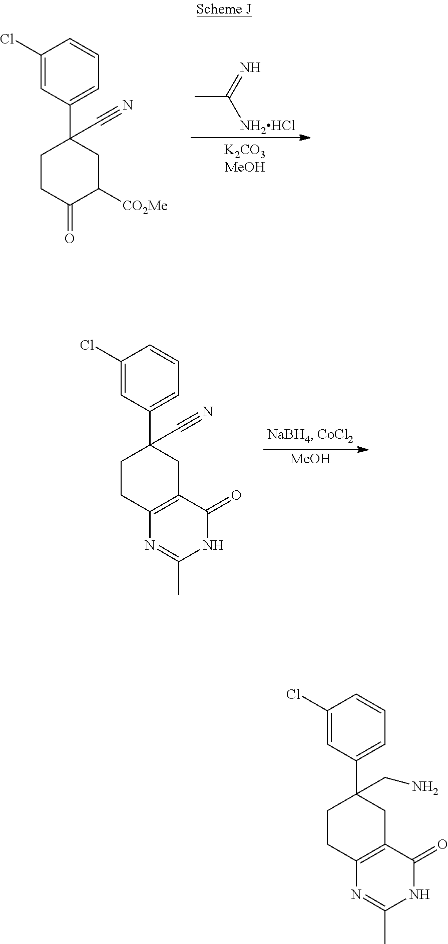

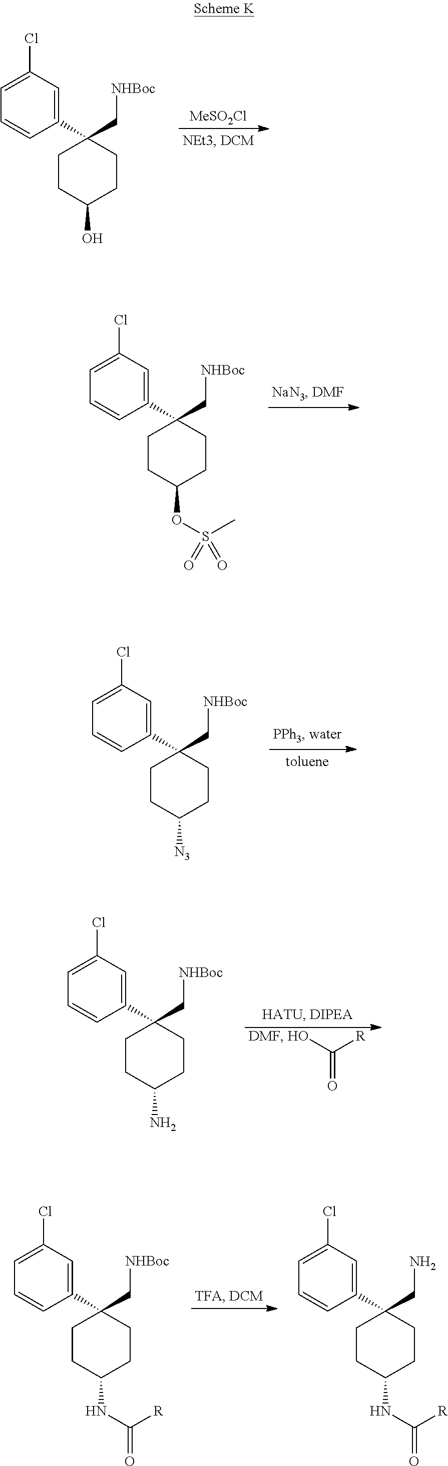

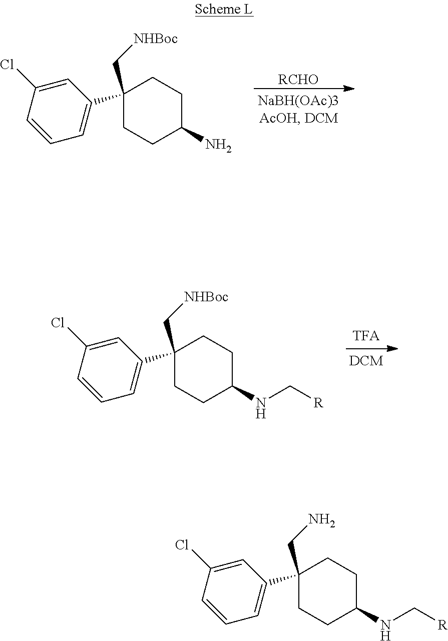

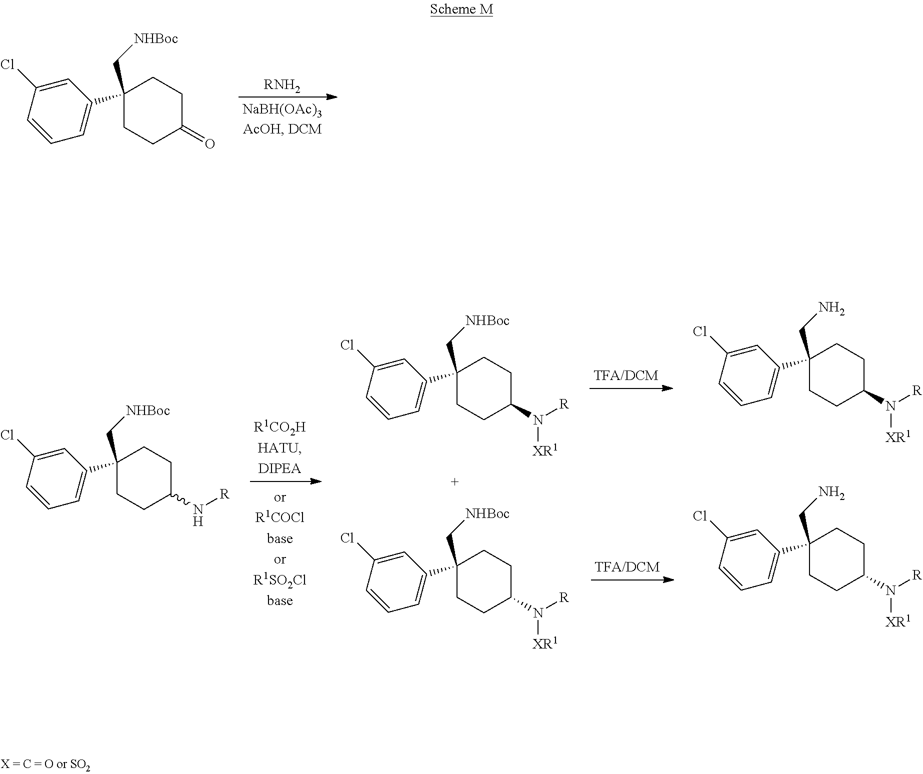

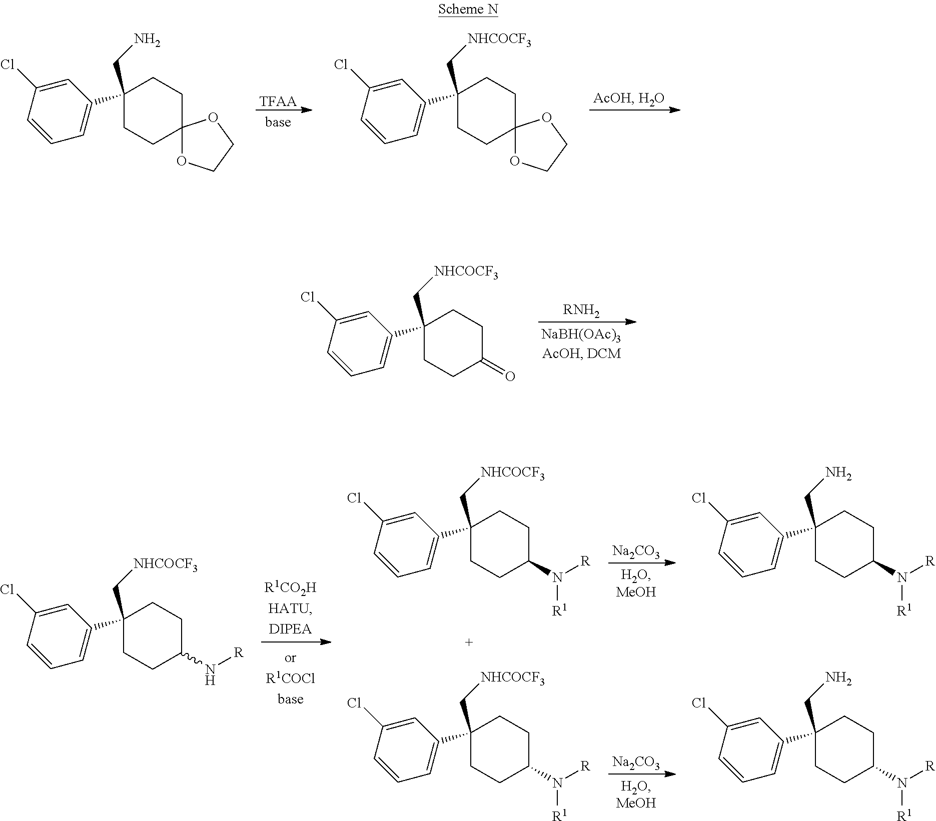

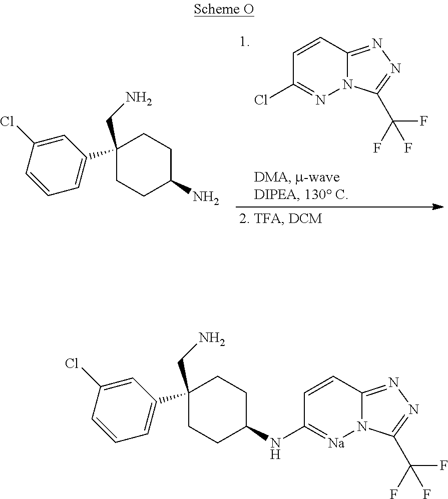

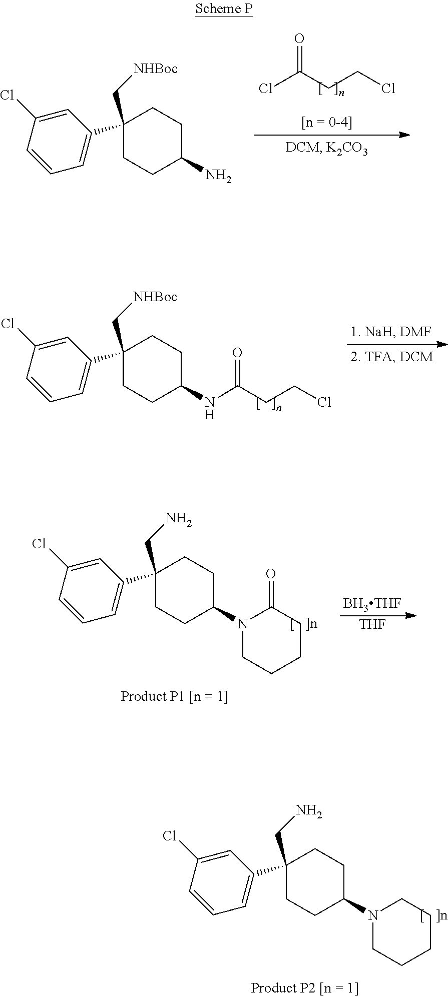

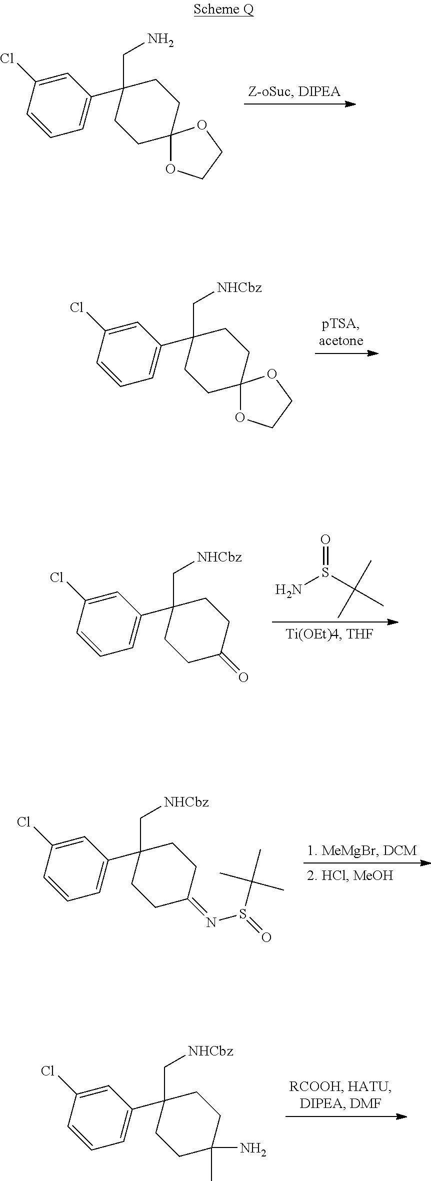

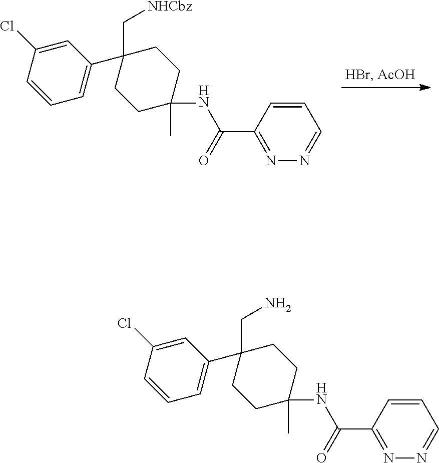 [SMILES: CC1(NC(=O)c2cccnn2)CCC(CNC(=O)OCc2ccccc2)(c2cccc(Cl)c2)CC1 ; CC1(NC(=O)c2cccnn2)CCC(CN)(c2cccc(Cl)c2)CC1]

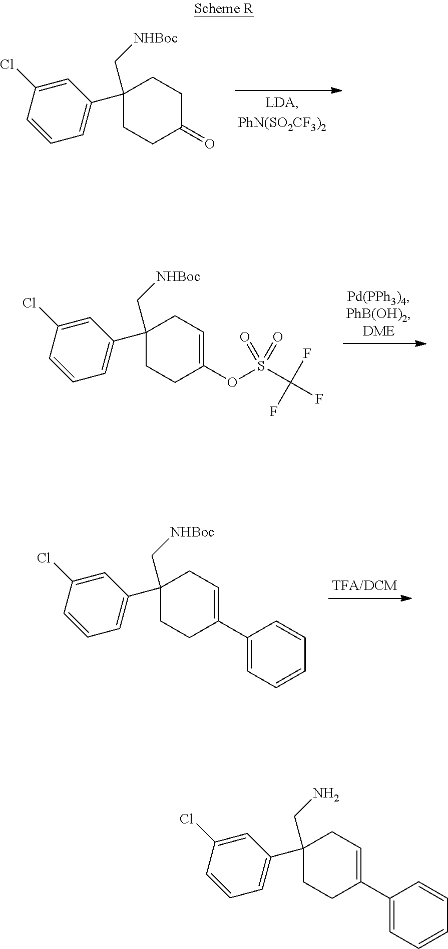 [SMILES: CC(C)(C)OC(=O)NCC1(c2cccc(Cl)c2)CCC(=O)CC1 ; CC(C)(C)OC(=O)NCC1(c2cccc(Cl)c2)CC=C(OS(=O)(=O)C(F)(F)F)CC1 ; CC(C)(C)OC(=O)NCC1(c2cccc(Cl)c2)CC=C(c2ccccc2)CC1 ; NCC1(c2cccc(Cl)c2)CC=C(c2ccccc2)CC1]

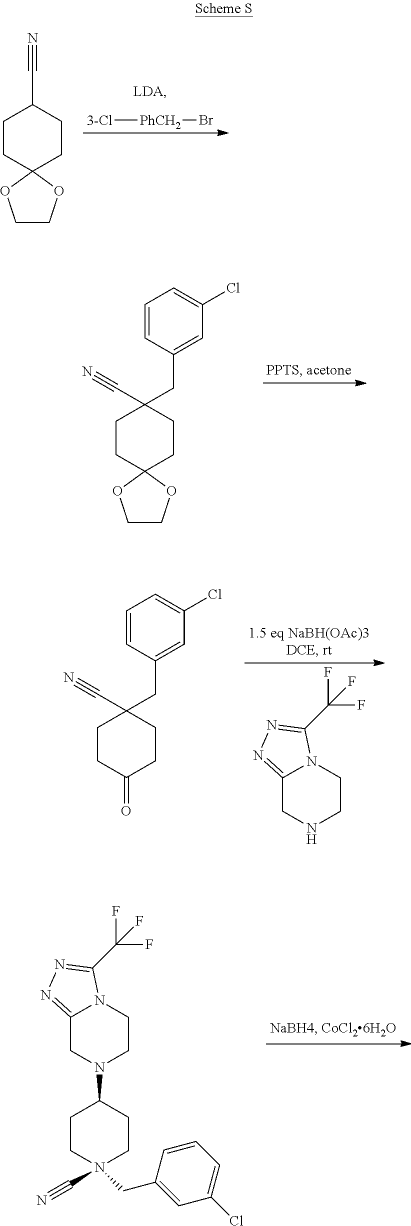

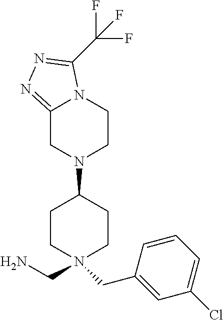

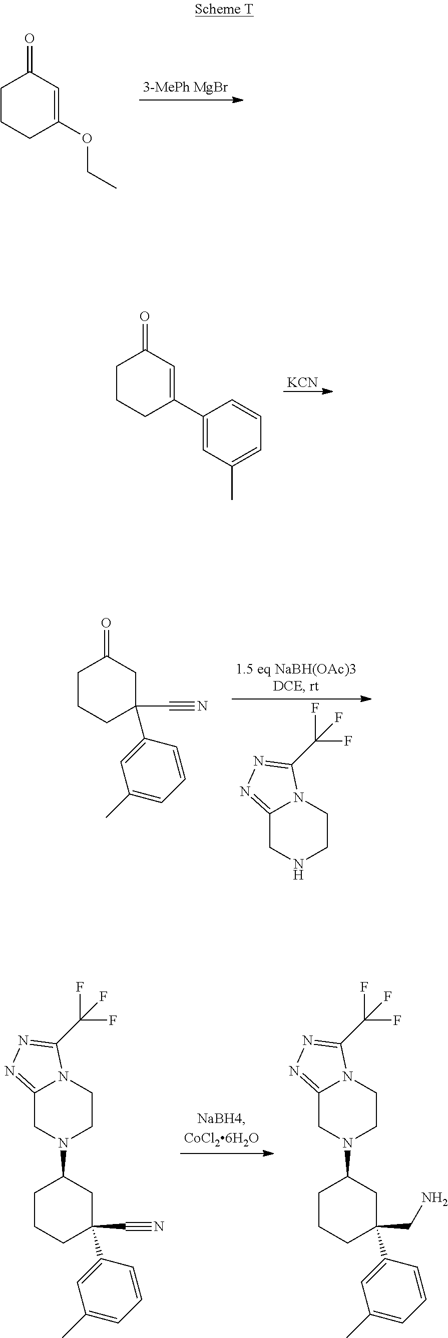

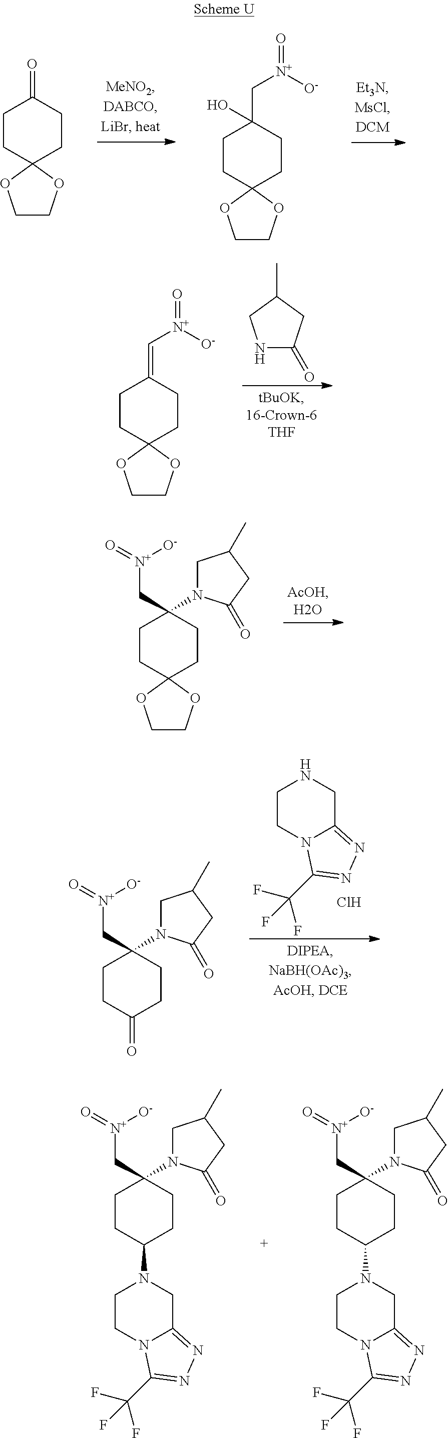

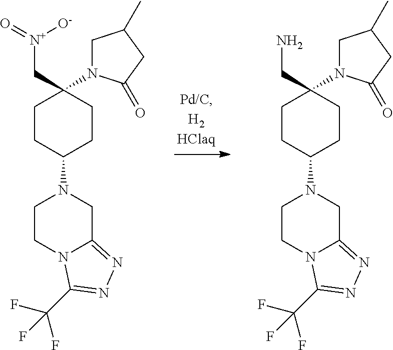 [SMILES: CC1CC(=O)N([C@]2(C[N+](=O)[O-])CC[C@H](N3CCn4c(nnc4C(F)(F)F)C3)CC2)C1 ; CC1CC(=O)N([C@]2(CN)CC[C@H](N3CCn4c(nnc4C(F)(F)F)C3)CC2)C1]

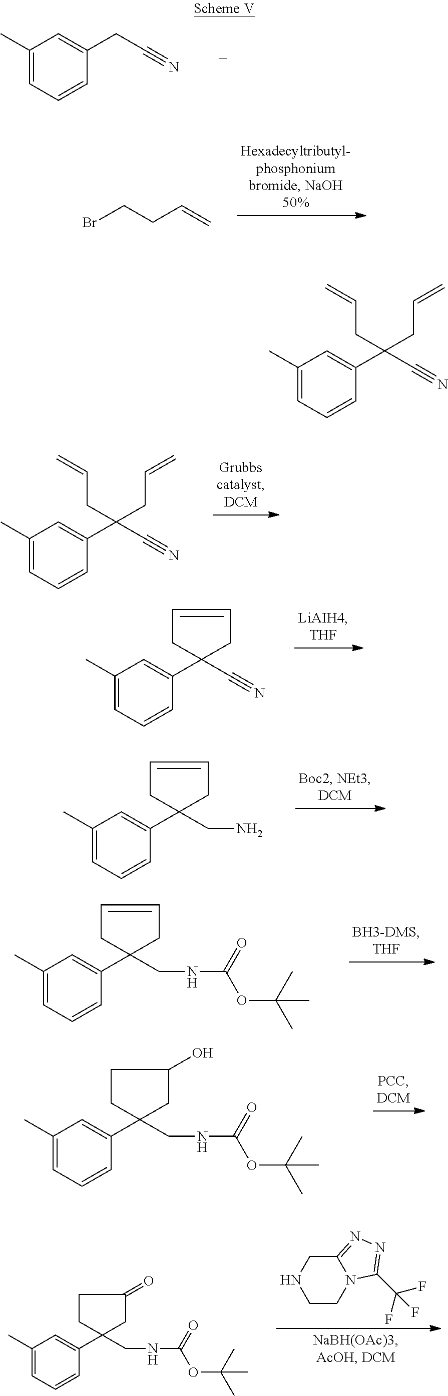

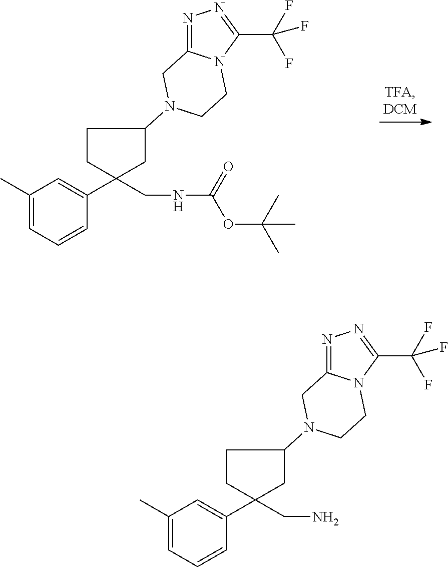 [SMILES: Cc1cccc(C2(CNC(=O)OC(C)(C)C)CCC(N3CCn4c(nnc4C(F)(F)F)C3)C2)c1 ; Cc1cccc(C2(CN)CCC(N3CCn4c(nnc4C(F)(F)F)C3)C2)c1]

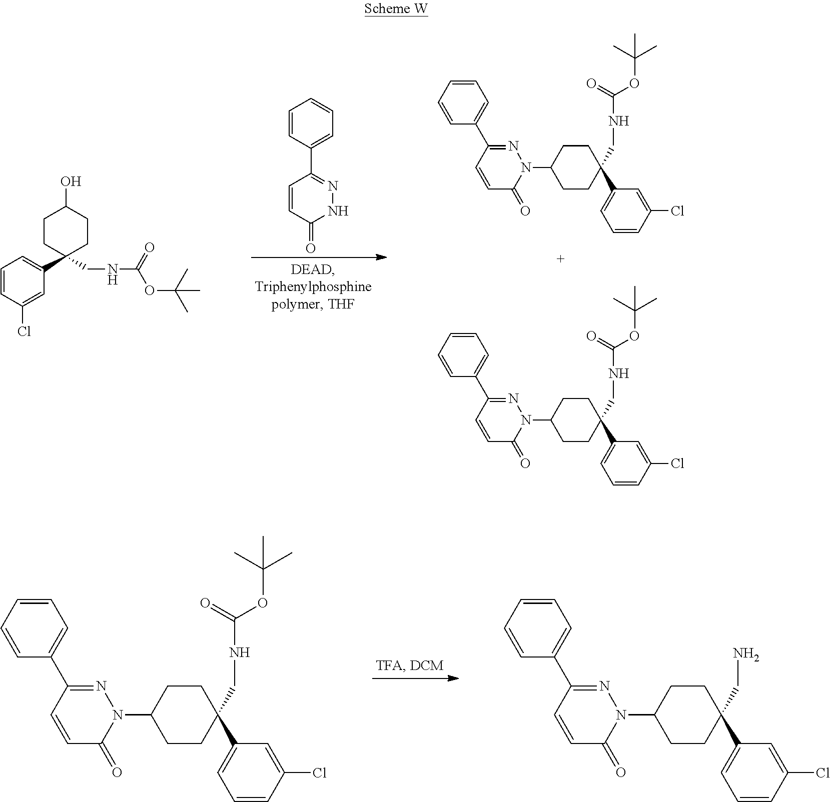

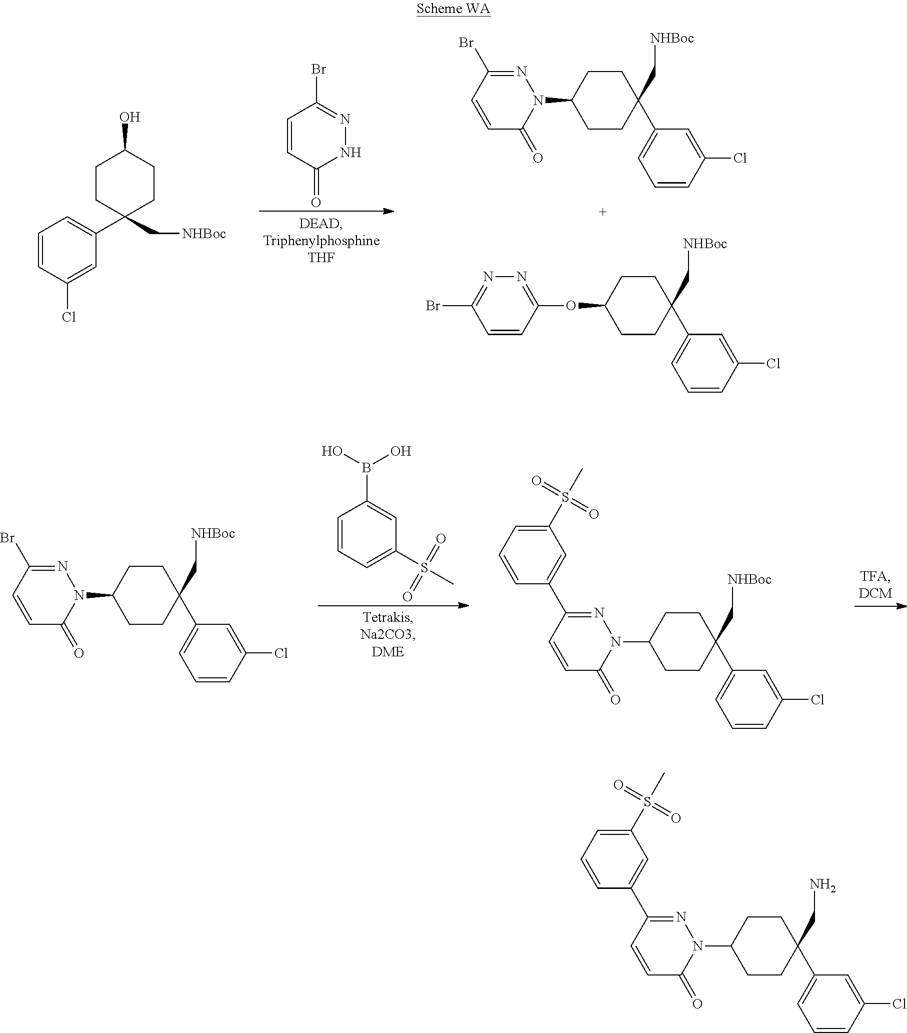

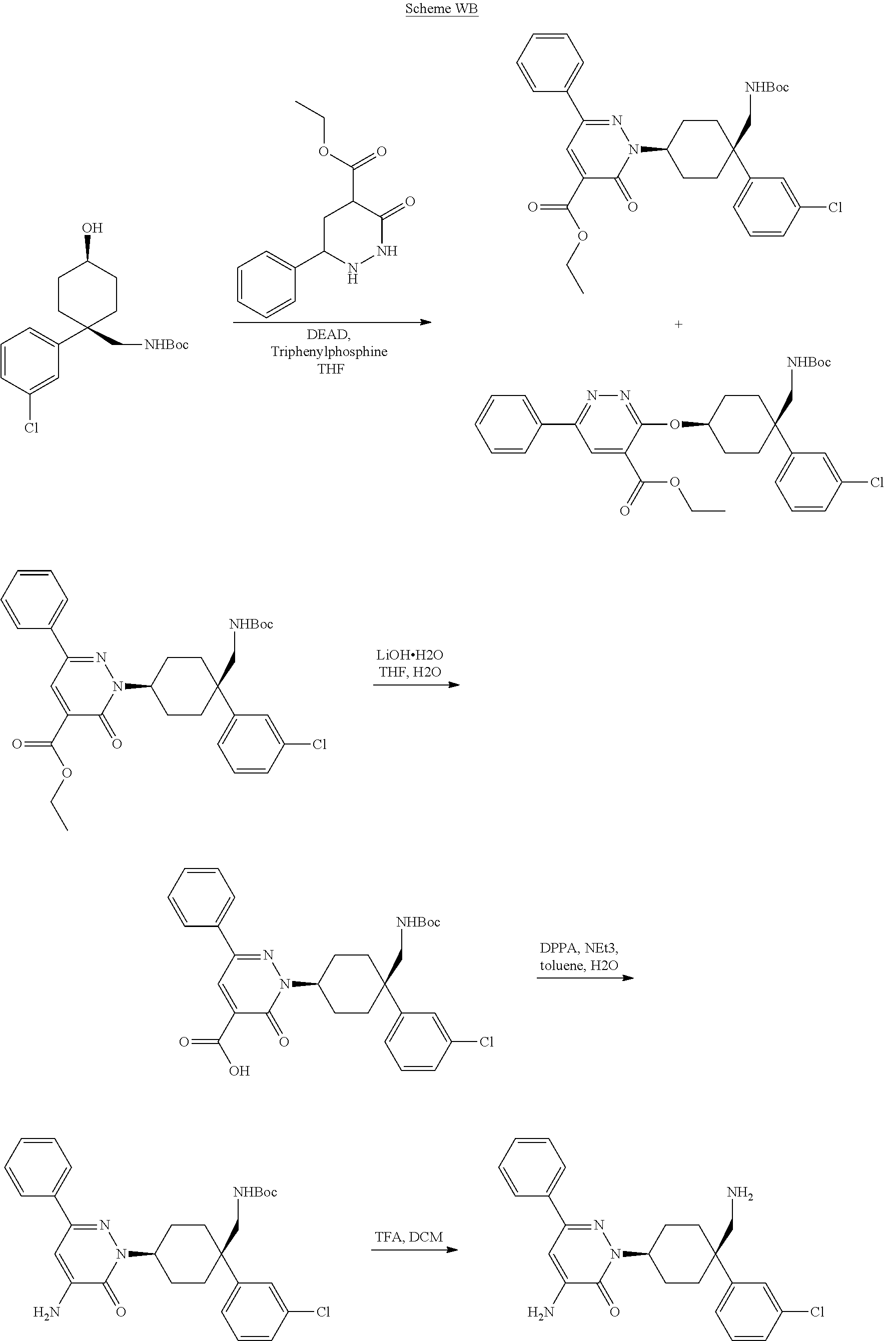

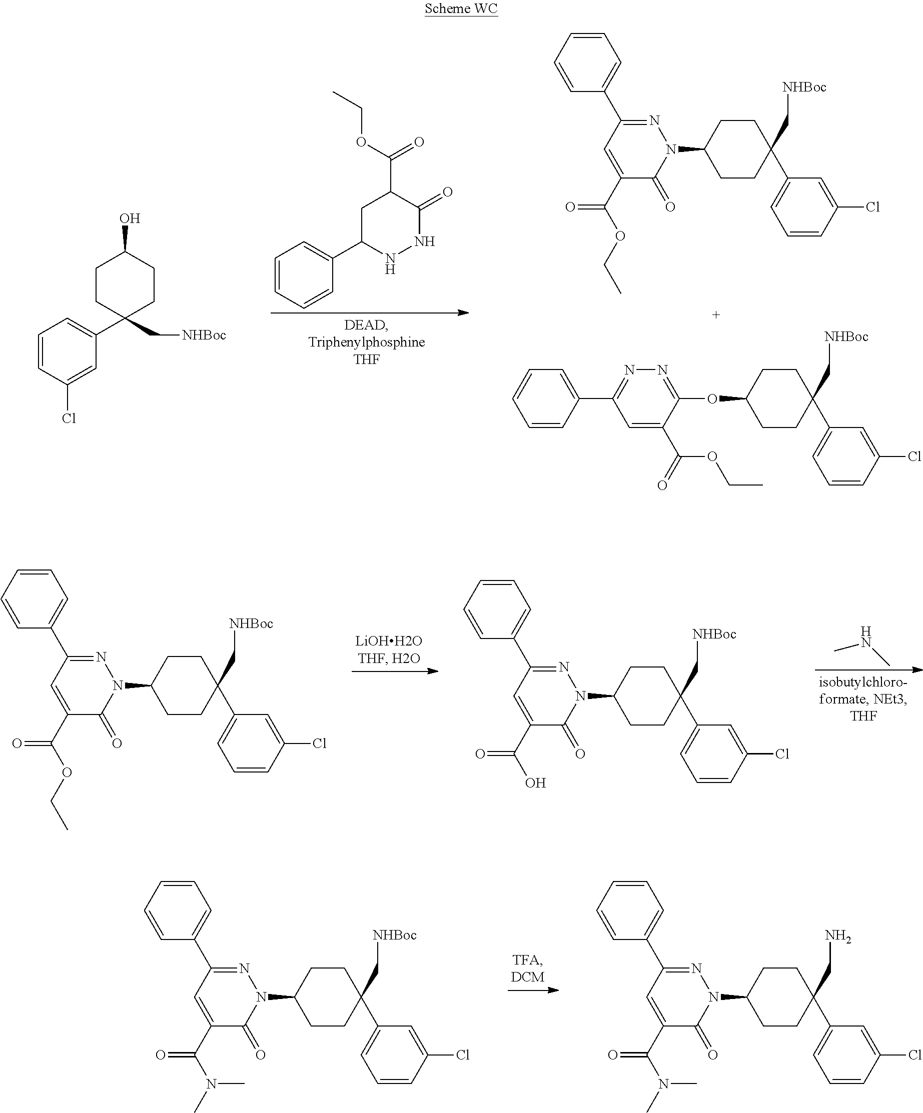

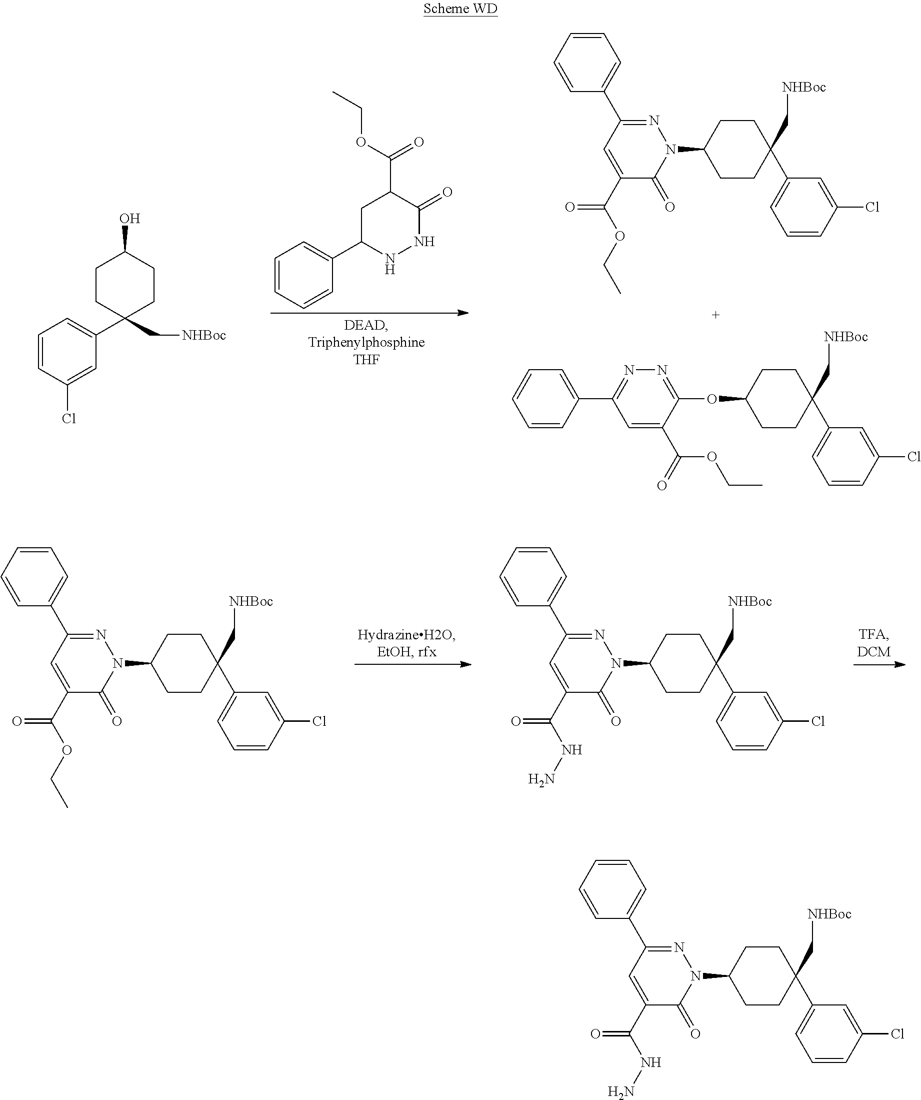

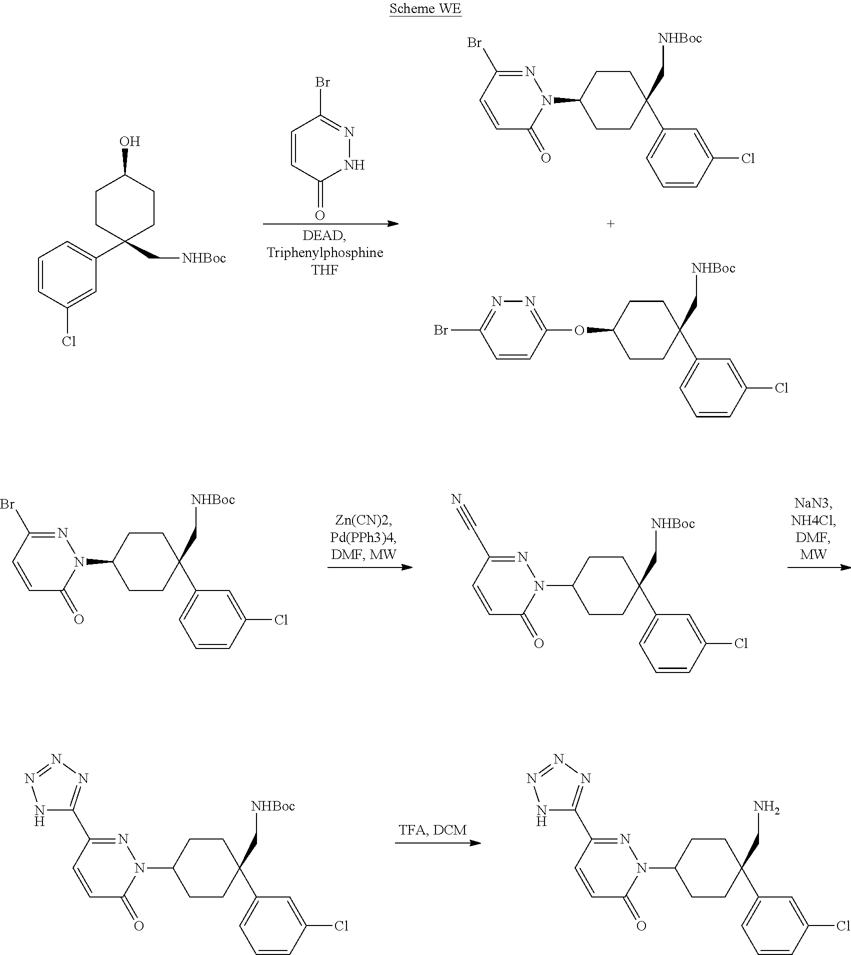

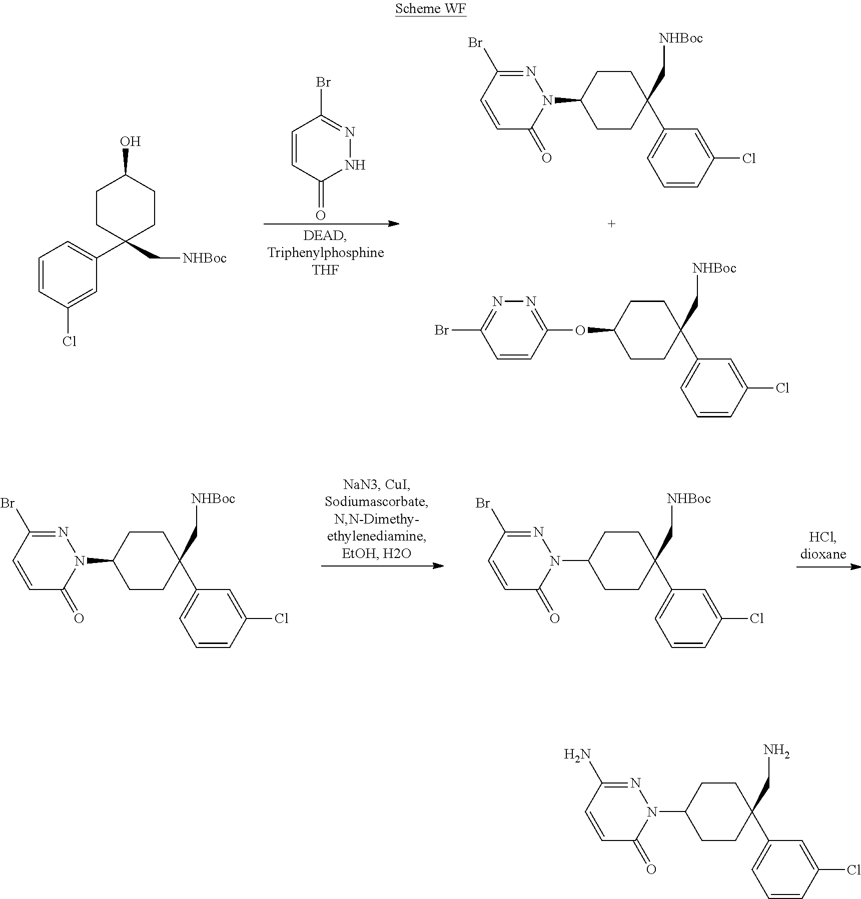

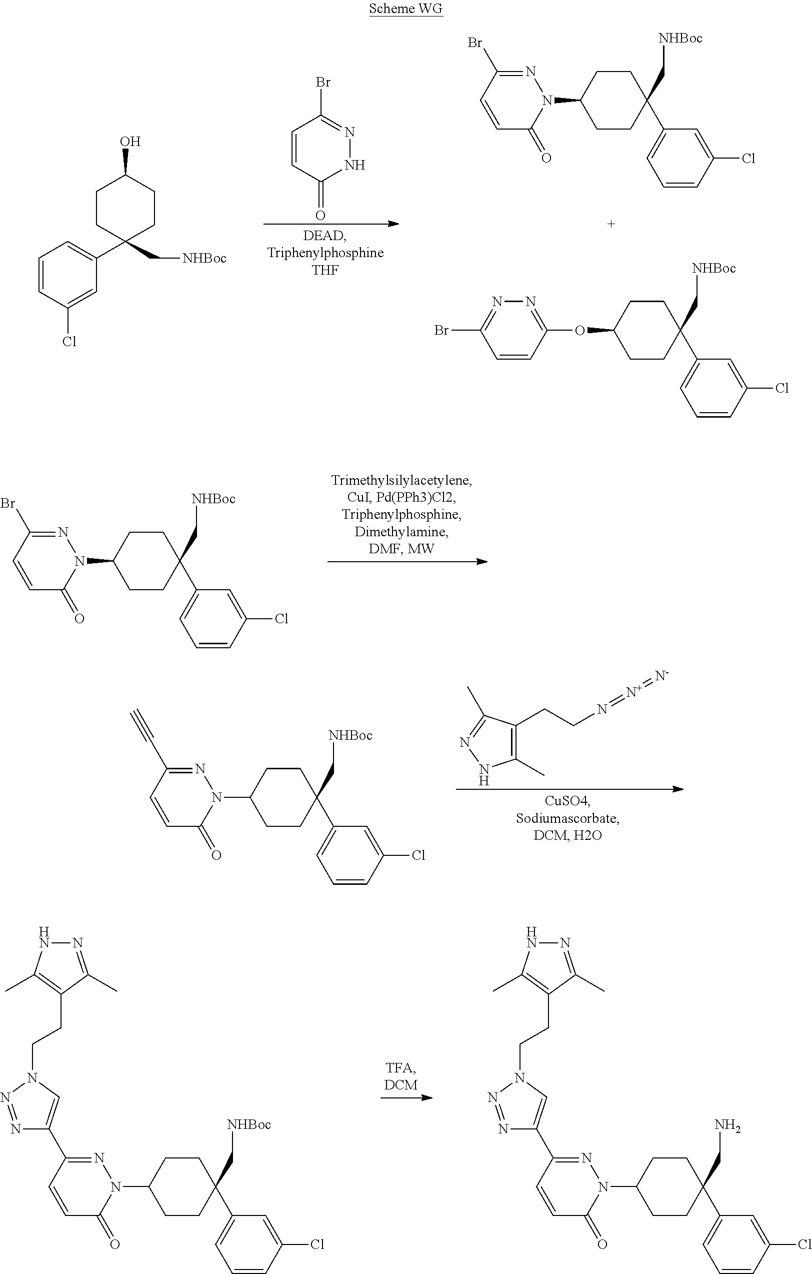

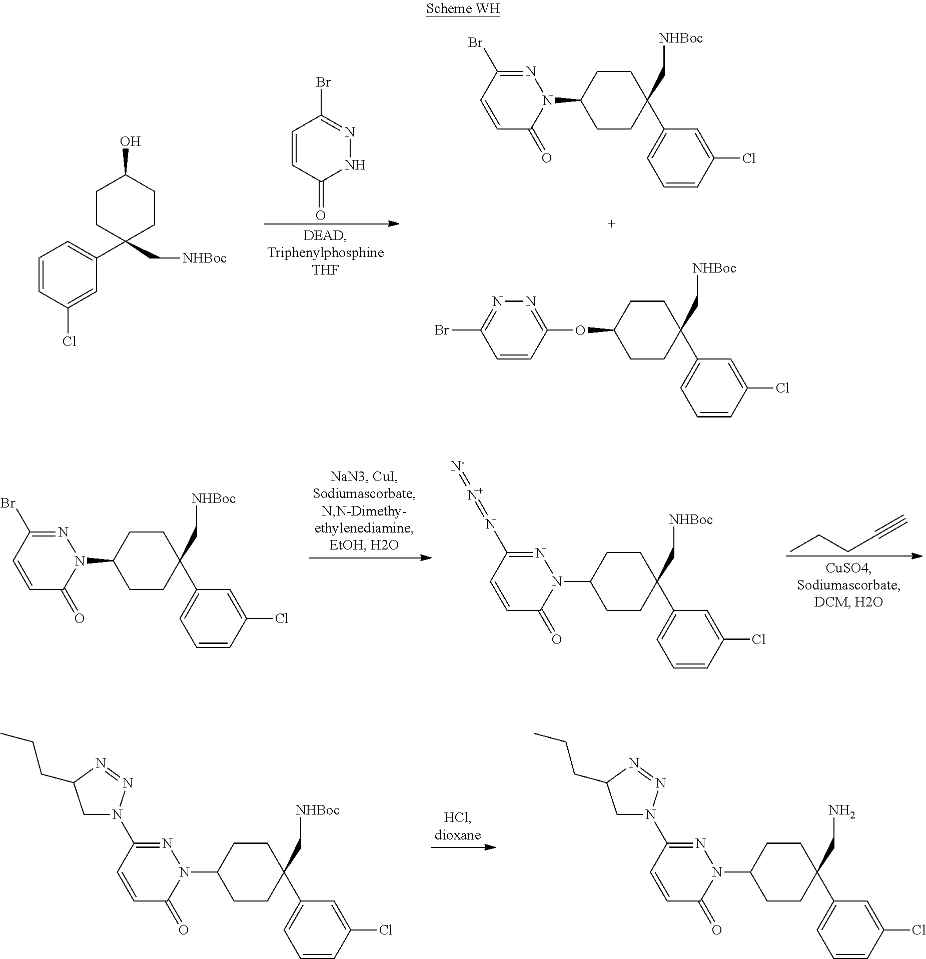

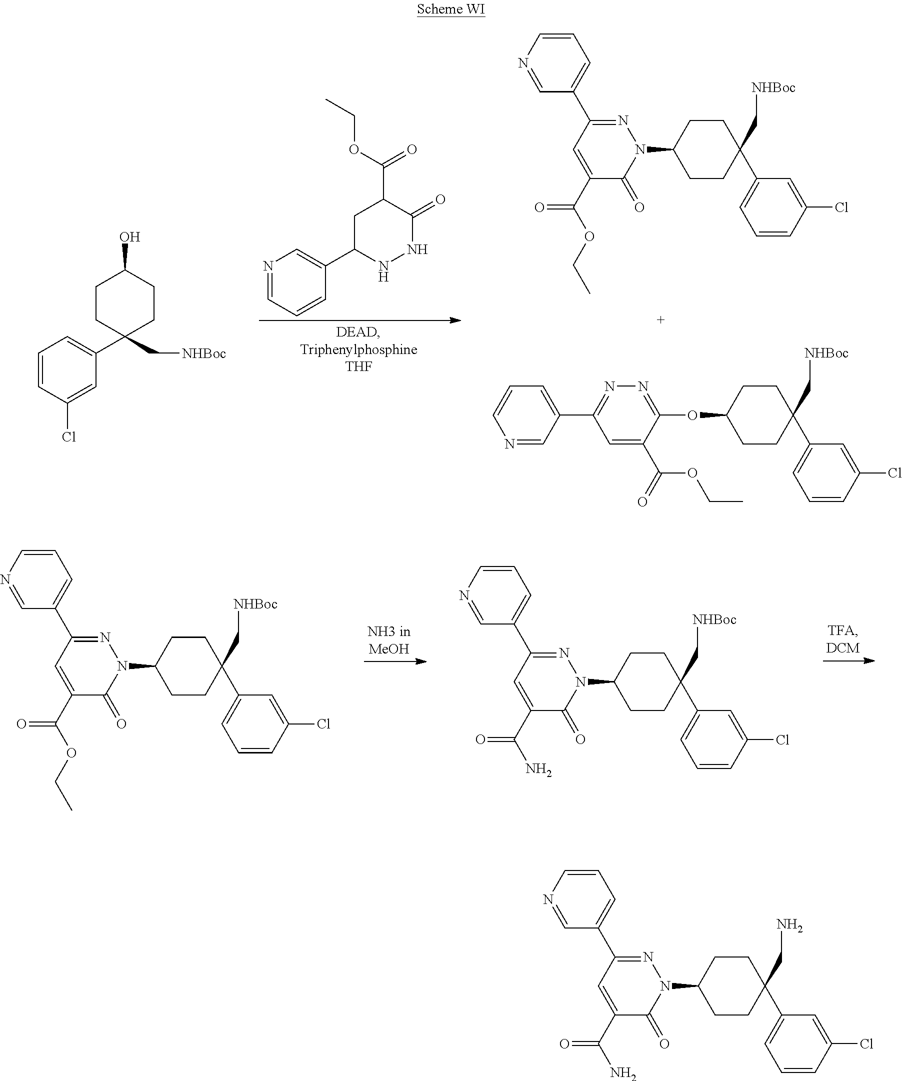

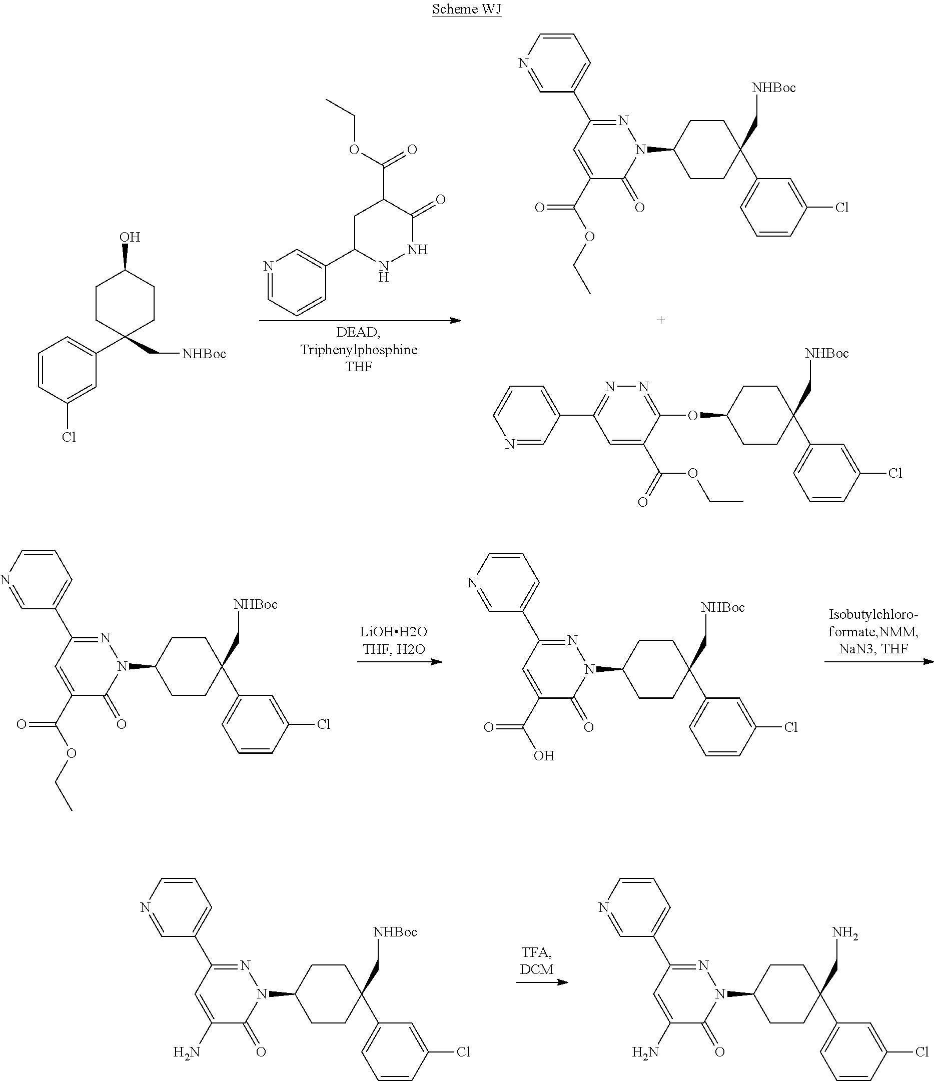

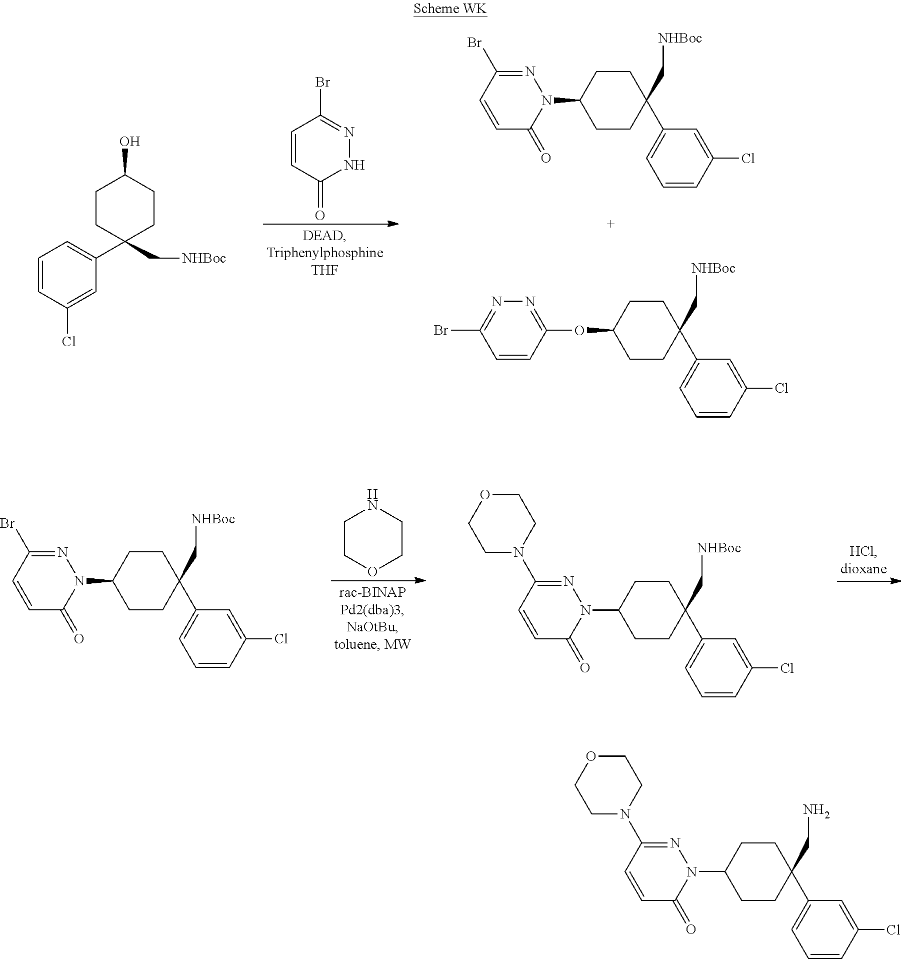

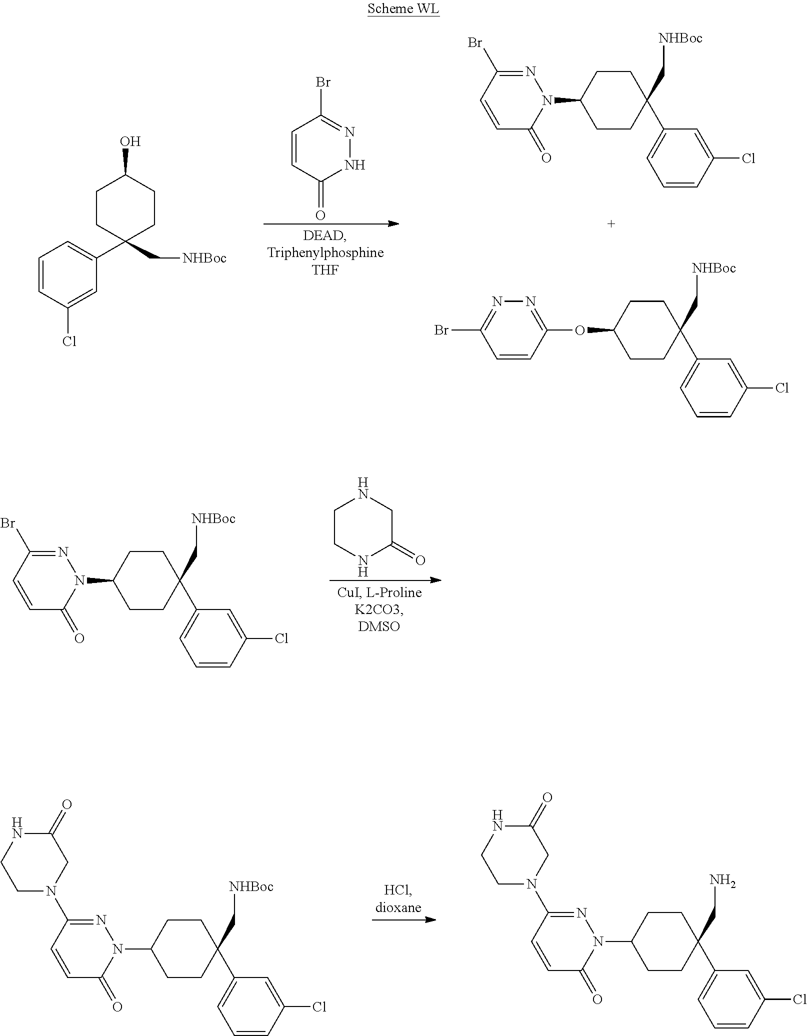

It will be understood that the processes detailed above and elsewhere herein are solely for the purpose of illustrating the invention and should not be construed as limiting. A process utilising similar or analogous reagents and/or conditions known to one skilled in the art may also be used to obtain a compound of the invention.

Any mixtures of final products or intermediates obtained can be separated on the basis of the physico-chemical differences of the constituents, in a known manner, into the pure final products or intermediates, for example by chromatography, distillation, fractional crystallisation, or by the formation of a salt if appropriate or possible under the circumstances.

### Administration & Pharmaceutical Formulations

The compounds of the invention will normally be administered orally, intravenously, subcutaneously, buccally, rectally, dermally, nasally, tracheally, bronchially, by any other parenteral route, as an oral or nasal spray or via inhalation, The compounds may be administered in the form of pharmaceutical preparations comprising prodrug or active compound either as a free compound or, for example, a pharmaceutically acceptable non-toxic organic or inorganic acid or base addition salt, in a pharmaceutically acceptable dosage form. Depending upon the disorder and patient to be treated and the route of administration, the compositions may be administered at varying doses.

Typically, therefore, the pharmaceutical compounds of the invention may be administered orally or parenterally (“parenterally” as used herein, refers to modes of administration which include intravenous, intramuscular, intraperitoneal, intrasternal, subcutaneous and intraarticular injection and infusion) to a host to obtain an protease-inhibitory effect. In the case of larger animals, such as humans, the compounds may be administered alone or as compositions in combination with pharmaceutically acceptable diluents, excipients or carriers.

Actual dosage levels of active ingredients in the pharmaceutical compositions of this invention may be varied so as to obtain an amount of the active compound(s) that is effective to achieve the desired therapeutic response for a particular patient, compositions, and mode of administration. The selected dosage level will depend upon the activity of the particular compound, the route of administration, the severity of the condition being treated and the condition and prior medical history of the patient being treated. However, it is within the skill of the art to start doses of the compound at levels lower than required for to achieve the desired therapeutic effect and to gradually increase the dosage until the desired effect is achieved.

In the treatment, prevention, control, amelioration, or reduction of risk of conditions which require inhibition of DPP-IV enzyme activity, an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0 and 1000.0 milligrams of the active ingredient for the symptomatic adjustment of the dosage to the patient to be treated. The compounds may be administered on a regimen of 1 to 4 times per day, preferably once or twice per day. The dosage regimen may be adjusted to provide the optimal therapeutic response.

According to a further aspect of the invention there is thus provided a pharmaceutical composition including a compound of the invention, in admixture with a pharmaceutically acceptable adjuvant, diluent or carrier.

Pharmaceutical compositions of this invention for parenteral injection suitably comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants.

These compositions may also contain adjuvants such as preservative, wetting agents, emulsifying agents and dispersing agents. Prevention of the action of microorganisms may be ensured by the inclusion of various antibacterial and antifungal agents, for example, paraben, chlorobutanol or phenol sorbic acid. It may also be desirable to include isotonic agents such as sugars or sodium chloride, for example. Prolonged absorption of the injectable pharmaceutical form may be brought about by the inclusion of agents (for example aluminum monostearate and gelatin) which delay absorption.

In some cases, in order to prolong the effect of the drug, it is desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are suitably made by forming microencapsule matrices of the drug in biodegradable polymers, for example polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations may also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use.

Solid dosage forms for oral administration include capsules, tablets, pills, powders and granules. In such solid dosage forms, the active compound is typically mixed with at least one inert, pharmaceutically acceptable excipient or carrier such as sodium citrate or dicalcium phosphate and/or one or more: a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycol, for example.

Suitably, oral formulations contain a dissolution aid. The dissolution aid is not limited as to its identity so long as it is pharmaceutically acceptable. Examples include nonionic surface active agents, such as sucrose fatty acid esters, glycerol fatty acid esters, sorbitan fatty acid esters (e.g. sorbitan trioleate), polyethylene glycol, polyoxyethylene hydrogenated castor oil, polyoxyethylene sorbitan fatty acid esters, polyoxyethylene alkyl ethers, methoxypolyoxyethylene alkyl ethers, polyoxyethylene alkylphenyl ethers, polyethylene glycol fatty acid esters, polyoxyethylene alkylamines, polyoxyethylene alkyl thioethers, polyoxyethylene polyoxypropylene copolymers, polyoxyethylene glycerol fatty acid esters, pentaerythritol fatty acid esters, propylene glycol monofatty acid esters, polyoxyethylene propylene glycol monofatty acid esters, polyoxyethylene sorbitol fatty acid esters, fatty acid alkylolamides, and alkylamine oxides; bile acid and salts thereof (e.g. chenodeoxycholic acid, cholic acid, deoxycholic acid, dehydrocholic acid and salts thereof, and glycine or taurine conjugate thereof); ionic surface active agents, such as sodium laurylsulfate, fatty acid soaps, alkylsulfonates, alkylphosphates, ether phosphates, fatty acid salts of basic amino acids; triethanolamine soap, and alkyl quaternary ammonium salts; and amphoteric surface active agents, such as betaines and aminocarboxylic acid salts.

The solid dosage forms of tablets, dragees, capsules, pills, and granules can be prepared with coatings and shells such as enteric coatings and other coatings well known in the pharmaceutical formulating art. They may optionally contain opacifying agents and may also be of a composition such that they release the active ingredient(s) only, or preferentially, in a certain part of the intestinal tract, and/or in delayed fashion. Examples of embedding compositions include polymeric substances and waxes.

The active compounds may also be in micro-encapsulated form, if appropriate, with one or more of the above-mentioned excipients.

The active compounds may be in finely divided form, for example it may be micronised.

Liquid dosage forms for oral administration include pharmaceutically acceptable emulsions, solutions, suspensions, syrups and elixirs. In addition to the active compounds, the liquid dosage forms may contain inert diluents commonly used in the art such as water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan and mixtures thereof. Besides inert diluents, the oral compositions may also include adjuvants such as wetting agents, emulsifying and suspending agents, sweetening, flavoring and perfuming agents. Suspensions, in addition to the active compounds, may contain suspending agents such as ethoxylated isostearyl alcohols, polyoxyethylene sorbitol and sorbitan esters, microcrystalline cellulose, aluminum metahydroxide, bentonite, agar-agar, and tragacanth and mixtures thereof.

Compositions for rectal or vaginal administration are preferably suppositories which can be prepared by mixing the compounds of this invention with suitable non-irritating excipients or carriers such as cocoa butter, polyethylene glycol or a suppository wax which are solid at room temperature but liquid at body temperature and therefore melt in the rectum or vaginal cavity and release the active compound.

Compounds of the present invention can also be administered in the form of liposomes. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolisable lipid capable of forming liposomes can be used. The present compositions in liposome form can contain, in addition to a compound of the present invention, stabilisers, preservatives, excipients and the like. The preferred lipids are the phospholipids and the phosphatidyl cholines (lecithins), both natural and synthetic. Methods to form liposomes are known in the art, for example, Prescott, Ed., Methods in Cell Biology, Volume XIV, Academic Press, New York, N.Y. (1976), p 33 et seq.

Dosage forms for topical administration of a compound of this invention include powders, sprays, ointments and inhalants. The active compound is mixed under sterile conditions with a pharmaceutically acceptable carrier and any needed preservatives, buffers or propellants which may be required. Ophthalmic formulations, eye ointments, powders and solutions are also contemplated as being within the scope of this invention.

Advantageously, the compounds of the invention may be orally active, have rapid onset of activity and low toxicity.

The compounds of the invention may have the advantage that they are more efficacious, less toxic, longer acting, have a broader range of activity, more potent, produce fewer side effects, more easily absorbed than, or have other useful pharmacological properties over, compounds known in the prior art.

### Combination Therapies

Compounds of the invention may be administered in combination with one or more additional therapeutic agents. Accordingly, the invention provides a pharmaceutical composition comprising an additional agent. The invention also provides a product comprising a compound of the invention and an agent; as a combined preparation for simultaneous, separate or sequential use in therapy.

In particular, a composition or product of the invention may further comprise a therapeutic agent selected from anti-diabetic agents, hypolipidemic agents, anti-obesity or appetite-regulating agents, anti-hypertensive agents, HDL-increasing agents, cholesterol absorption modulators, Apo-A1 analogues and mimetics, thrombin inhibitors, aldosterone inhibitors, inhibitors of platelet aggregation, estrogen, testosterone, selective estrogen receptor modulators, selective androgen receptor modulators, chemotherapeutic agents, and 5-HT₃ or 5-HT₄ receptor modulators; or pharmaceutically acceptable salts or prodrugs thereof.

Examples of anti-diabetic agents include insulin, insulin derivatives and mimetics; insulin secretagogues, for example sulfonylureas (e.g. glipizide, glyburide or amaryl); insulinotropic sulfonylurea receptor ligands, for example meglitinides (e.g. nateglinide or repaglinide); insulin sensitisers, for example protein tyrosine phosphatase-1B (PTP-1B) inhibitors (e.g. PTP-112); GSK3 (glycogen synthase kinase-3) inhibitors, for example SB-517955, SB-4195052, SB-216763, N,N-57-05441 or N,N-57-05445; RXR ligands, for example GW-0791 or AGN-194204; sodium-dependent glucose cotransporter inhibitors, for example T-1095; glycogen phosphorylase A inhibitors, for example BAY R3401; biguanides, for example metformin; alpha-glucosidase inhibitors, for example acarbose; GLP-1 (glucagon like peptide-1), GLP-1 analogues and mimetics, for example exendin-4; DPPIV (dipeptidyl peptidase IV) inhibitors, for example DPP728, LAF237 (vildagliptin), MK-0431, saxagliptin or GSK23A; AGE breakers; and thiazolidone derivatives, for example glitazone, pioglitazone, rosiglitazone or (R)-1-{4-[5-methyl-2-(4-trifluoromethyl-phenyl)-oxazol-4-ylmethoxy]-benzenesulfonyl}-2,3-dihydro-1H-indole-2-carboxylic acid (compound 4 of Example 19 of WO 03/043985) or a non-glitazone type PPAR-agonist (e.g. GI-262570); or pharmaceutically acceptable salts or prodrugs thereof.

Examples of hypolipidemic agents include 3-hydroxy-3-methyl-glutaryl coenzyme A (HMG-CoA) reductase inhibitors, for example lovastatin, pitavastatin, simvastatin, pravastatin, cerivastatin, mevastatin, velostatin, fluvastatin, dalvastatin, atorvastatin, rosuvastatin or rivastatin; squalene synthase inhibitors; FXR (farnesoid X receptor) ligands; LXR (liver X receptor) ligands; cholestyramine; fibrates; nicotinic acid; and aspirin; or pharmaceutically acceptable salts or prodrugs thereof.

Examples of anti-obesity/appetite-regulating agents include phentermine, leptin, bromocriptine, dexamphetamine, amphetamine, fenfluramine, dexfenfluramine, sibutramine, orlistat, dexfenfluramine, mazindol, phentermine, phendimetrazine, diethylpropion, fluoxetine, bupropion, topiramate, diethylpropion, benzphetamine, phenylpropanolamine or ecopipam, ephedrine, pseudoephedrine and cannabinoid receptor antagonists; or pharmaceutically acceptable salts or prodrugs thereof.

Examples of anti-hypertensive agents include loop diuretics, for example ethacrynic acid, furosemide or torsemide; diuretics, for example thiazide derivatives, chlorithiazide, hydrochlorothiazide or amiloride; angiotensin converting enzyme (ACE) inhibitors, for example benazepril, captopril, enalapril, fosinopril, lisinopril, moexipril, perinodopril, quinapril, ramipril or trandolapril; Na—K-ATPase membrane pump inhibitors, for example digoxin; neutralendopeptidase (NEP) inhibitors, for example thiorphan, terteo-thiorphan or SQ29072; ECE inhibitors, for example SLV306; dual ACE/NEP inhibitors, for example omapatrilat, sampatrilat or fasidotril; angiotensin II antagonists, for example candesartan, eprosartan, irbesartan, losartan, telmisartan or valsartan; renin inhibitors, for example aliskiren, terlakiren, ditekiren, RO-66-1132 or RO-66-1168; b-adrenergic receptor blockers, for example acebutolol, atenolol, betaxolol, bisoprolol, metoprolol, nadolol, propranolol, sotalol or timolol; inotropic agents, for example digoxin, dobutamine or milrinone; calcium channel blockers, for example amlodipine, bepnaii, dimazem, felodipine, nicardipine, nimodipine, nifedipine, nisoldipine or verapamil; aldosterone receptor antagonists; and aldosterone synthase inhibitors; or pharmaceutically acceptable salts or prodrugs thereof.

Examples of cholesterol absorption modulators include Zetia® and KT6-971, or pharmaceutically acceptable salts or prodrugs thereof.

Examples of aldosterone inhibitors include anastrazole, fadrazole and eplerenone, or pharmaceutically acceptable salts or prodrugs thereof.

Examples of inhibitors of platelet aggregation include aspirin or clopidogrel bisulfate, or pharmaceutically acceptable salts or prodrugs thereof.

Examples of chemotherapeutic agents include compounds decreasing the protein kinase activity, for example PDGF receptor tyrosine kinase inhibitors (e.g. imatinib or 4-methyl-N-[3-(4-methyl-imidazol-1-yl)-5-trifluoromethyl-phenyl]-3-(4-pyridin-3-yl-pyrimidin-2-ylamino)-benzamide), or pharmaceutically acceptable salts or prodrugs thereof.

Examples of 5-HT₃ or 5-HT₄ receptor modulators include tegaserod, tegaserod hydrogen maleate, cisapride or cilansetron, or pharmaceutically acceptable salts or prodrugs thereof.

The weight ratio of the compound of the present invention to the further active ingredient(s) may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, preferably about 200:1 to about 1:200.

Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

### Use

Compounds of the invention may be useful in the therapy of a variety of diseases and conditions.

In particular, compounds of the invention may be useful in the treatment or prevention of a disease or condition selected from non-insulin-dependent diabetes mellitus, arthritis, obesity, allograft transplantation, osteoporosis, heart failure, impaired glucose metabolism or impaired glucose tolerance, neurodegenerative diseases (for example Alzheimer's disease or Parkinson disease), cardiovascular or renal diseases (for example diabetic cardiomyopathy, left or right ventricular hypertrophy, hypertrophic medial thickening in arteries and/or in large vessels, mesenteric vasculature hypertrophy or mesanglial hypertrophy), neurodegenerative or cognitive disorders, hyperglycemia, insulin resistance, lipid disorders, dyslipidemia, hyperlipidemia, hypertriglyceridemia, hypercholesterolemia, low HDL levels, high LDL levels, atherosclerosis, vascular restenosis, irritable bowel syndrome, inflammatory bowel disease (e.g. Crohn's disease or ulcerative colitis), pancreatitis, retinopathy, nephropathy, neuropathy, syndrome X, ovarian hyperandrogenism (polycystic ovarian syndrome), type 2 diabetes, growth hormone deficiency, neutropenia, neuronal disorders, tumor metastasis, benign prostatic hypertrophy, gingivitis, hypertension and osteoporosis.

The compounds may also be useful in producing a sedative or anxiolytic effect, attenuating post-surgical catabolic changes or hormonal responses to stress, reducing mortality and morbidity after myocardial infarction, modulating hyperlipidemia or associated conditions; and lowering VLDL, LDL or Lp(a) levels.

### EXAMPLES

The following Examples illustrate the invention.

### Example A1

### 4-Aminomethyl-4-(2,5-difluoro-phenyl)-cyclohexanol

This compound was prepared according to Scheme A:

### A) 4-(2,5-Difluoro-phenyl)-4-cyano-heptanedioic acid di-tert-butyl ester

A solution of 2,5-difluorobenzyl cyanide (2.00 g, 13.06 mmol) and tert-butyl acrylate (9.86 ml, 67.92 mmol) in t-BuOH (20 ml) was heated at 60° C. The heat was quickly removed and a solution of Triton B (1.98 ml of 40% MeOH solution diluted with 10 ml of tBuOH, 4.4 mmol) was added in one portion. The mixture was stirred at reflux for 5 h then cooled to RT. The mixture was diluted with Et₂O (300 ml) and washed successively with 2M aqueous HCl solution (150 ml) and brine (150 ml). The organic layer was dried over Na₂SO₄, filtered, then evaporated. The crude material was purified by silica gel chromatography (gradient elution, hexane/TBME 95:5 to 3:7) to provide the title compound (3.44 g).

MS: 427.6 [M+H₂O]⁺

HPLC (SunFire TM (4.6×20 mm) C18, 3.5 μm, 3 ml/min, linear gradient MeCN in H₂O (0.1% TFA) 5 to 100% in 4 min then 0.5 min 100%): Rt=3.18 min

### B) 5-(2,5-difluoro-phenyl)-5-cyano-2-oxo-cyclohexanecarboxylic acid tert-butyl ester

A solution of 4-(2,5-difluoro-phenyl)-4-cyano-heptanedioic acid di-tert-butyl ester (3.13 g, 7.49 mmol) in THF (60 ml) was treated with t-BuOK (1.73 g 15.0 mmol) at RT then the mixture was refluxed for 5 h. The reaction was then cooled to 0° C. in ice-bath, acidified by addition of AcOH—H₂O (2.14 ml in 20 ml) and diluted with Et₂O (150 ml). The organic layer was separated then washed successively with 1M Na₂CO₃ aqueous solution (2×50 ml), water (2×50 ml), and brine (50 ml). The organic layer was dried over Na₂SO₄, filtered, and evaporated to obtain the title compound as a crude (2.91 g).

MS: 336.2 [M+H]⁺, 353.2 [M+H₂O]⁺

HPLC (SunFire TM (4.6×20 mm) C18, 3.5 μm, 3 ml/min, linear gradient MeCN in H₂O (0.1% TFA) 5 to 100% in 4 min then 0.5 min 100%): Rt=2.95 min

### C) 1-(2,5-Difluoro-phenyl)-4-oxo-cyclohexanecarbonitrile

A mixture of 5-(2,5-difluoro-phenyl)-5-cyano-2-oxo-cyclohexanecarboxylic acid tert-butyl ester (crude 2.91 g, ca. 7.49 mmol) and NaCl (2.63 g, 44.9 mmol) in DMSO (60 ml) and water (4 ml) was heated at 150° C. for 5 h. The reaction was then cooled to RT, diluted with Et₂O (500 ml) and washed with 1N aqueous HCl(2×200 ml) and brine (50 ml). The organic layer was dried over Na₂SO₄, filtered and evaporated. The remaining oil was purified with silica gel chromatography (gradient elution, hexane:TBME 95:5 to 1:1) to provide a mixture containing the title compound. This mixture was sublimed in a Kugelrohr apparatus (140° C., 0.017 mbar) to yield the pure title compound as a colorless solid (554 mg).

HPLC (SunFire TM (4.6×20 mm) C18, 3.5 μm, 3 ml/min, linear gradient MeCN in H₂O (0.1% TFA) 5 to 100% in 4 min then 0.5 min 100%): Rt=1.74 min

### D) 1-(2,5-Difluoro-phenyl)-4-hydroxy-cyclohexanecarbonitrile

To a solution of 1-(2,5-difluoro-phenyl)-4-oxo-cyclohexanecarbonitrile (150 mg, 0.625 mmol) in dry THF (2 ml) was added at −78° C. NaBH₄ (49 mg, 1.25 mmol), and the reaction was stirred at −78° C. for 1 hr before carefully quenched by MeOH. EtOAc was added and the phases are separated. The aqueous phase was further extracted twice with EtOAc. The combined organic phase was washed once with brine, dried over Na₂SO₄, filtered and evaporated. The crude product was purified with silica gel chromatography (gradient elution, hexane-CH₂Cl₂ (1:1)/TBME 95/5 to 6/4) to yield the title compound (114 mg, 0.48 mmol).

MS: 256.26 [M+H₂O]⁺

TLC (silica gel, hexane:CH₂Cl₂:TBME 1:1:2): Rf=0.35

### E) 4-Aminomethyl-4-(2,5-difluoro-phenyl)-cyclohexanol

To a solution of 1-(2,5-difluoro-phenyl)-4-hydroxy-cyclohexanecarbonitrile (50 mg, 0.211 mmol) in dry THF (1 ml) was added BH₃ (1M solution in THF, 2.1 ml, 2.1 mmol), and the reaction flask was sealed and heated at 70° C. for 20 h. After cooled to RT, the reaction was carefully quenched by addition of MeOH then evaporated. The crude product was purified by preparative HPLC to yield the title compound as a TFA salt (19.4 mg, 0.055 mmol).

MS: 242.3 [M+H]+

HPLC (SunFire TM (4.6×20 mm) C18, 3.5 μm, 3 ml/min, linear gradient MeCN in H₂O (0.1% TFA) 5 to 100% in 4 min then 0.5 min 100%): Rt=0.87 min

### Example A2

### 4-Aminomethyl-4-phenyl-cyclohexanol

The title compound was prepared analogously as described in example A1 using Benzylcyanide instead of 2,5-difluorobenzyl cyanide.

MS: 206 [M+H]⁺

### Example B1

### C-[1-(2,5-Difluoro-phenyl)-4-methoxy-cyclohexyl]-methylamine

This compound was prepared according to Scheme B:

### A) 1-(2,5-Difluoro-phenyl)-4-methoxy-cyclohexanecarbonitrile

To NaH (67 mg, 60% in mineral oil, 1.68 mmol, washed with hexane, suspended in dry THF 1 ml) were added 1-(2,5-difluoro-phenyl)-4-hydroxy-cyclohexanecarbonitrile (100 mg, 0.421 mmol) in dry THF (1 ml) and MeI (0.105 ml, 1.68 mmol). The reaction was stirred at it for 2 hrs then carefully quenched with sat. NH₄Cl aq., and extracted twice with ethyl acetate. The combined organic phase was washed with brine, dried over Na₂SO₄ and evaporated in vacuo to give 87 mg of the title compound as a pale yellow solid.

TLC (silicagel, cyclohexane:acetone 3:2): Rf=0.57.

### B) C-[1-(2,5-Difluoro-phenyl)-4-methoxy-cyclohexyl]-methylamine

To a solution of 1-(2,5-difluoro-phenyl)-4-methoxy-cyclohexanecarbonitrile (87 mg, 0.346 mmol) in dry THF (1 ml) was added LiAlH₄ (22.6 mg, 0.578 mmol), and the reaction was stirred at 50° C. for 1 hr. After careful quench with sat. NH₄Cl aq., the mixture was extracted three times with ethyl acetate, and the combined organic phase was washed with brine, dried over Na₂SO₄ and evaporated. The residual oil was taken up in MeOH-MeCN (1:1) and loaded over 6 ml SCX column filled with benzenesulfonic acid (500 mg), eluted with ethyl acetate and methanol. Finally the amine was washed off with 2M ammonia in methanol. Evaporation of the amine solution in vacuo gives a white solid which was further purified by preparative HPLC to afford pure title compound as a white solid (10 mg).

MS: 256.1 [M+H]⁺

HPLC(WATERS Symmetry C18, linear gradient MeCN in H₂O (0.1% formic acid) 20% (0-1 min), 20-100% (1-6 min), 100% (6-8.5 min)): Rt=3.42 min

### Example B2

### C-[1-Phenyl-4-((E)-3-phenyl-allyloxy)-cyclohexyl]-methylamine

The title compound was prepared analogously as described in example B2 step A) using commercially available 4-cyano-4-phenyl-cyclohexanone and ((E)-3-bromo-propenyl)-benzene instead of 1-(2,5-difluoro-phenyl)-4-hydroxy-cyclohexanecarbonitrile and MeI, respectively.

MS: 322.15 [M+H]⁺

### Example B3

### 1-[cis-1-(3-Chlorophenyl)-4-methoxycyclohexyl]methanamine hydrochloride

This compound was prepared by adaptation of the route shown in Scheme B.

### A) cis-1-(3-Chlorophenyl)-4-hydroxy-cyclohexanecarbonitrile

1-(3-Chlorophenyl)-4-oxo-cyclohexanecarbonitrile (530 mg, 2.3 mmol) was dissolved in tetrahydrofuran (7 mL) and cooled to −78° C. under an atmosphere of nitrogen. Sodium borohydride (170 mg, 4.5 mmol) was added and the reaction mixture was stirred at −78° C. for 1.5 hours. The reaction was quenched by the addition of methanol (10 mL) and diluted with ethyl acetate (20 mL). The layers were separated and the aqueous layer was extracted with a more ethyl acetate (20 mL). The combined organic phases were washed with water (2×20 mL) and brine (2×20 mL), dried (MgSO₄), and concentrated to a yellow gum. The gum was purified by flash chromatography (Silica, eluting with 20% ethyl acetate in cyclohexane) to afford the title compound as a white sticky solid.

MS (ES⁺): 236 [M+H]⁺.

T_{R} [HPLC, Phenomenex Luna 3 micron C18; 5-95% CH₃CN+0.1% Formic acid/H₂O+0.1%

Formic acid for 5 min, flow 2.0 ml/min]: 3.04 min.

### B) cis-1-(3-Chlorophenyl)-4-methoxy-cyclohexanecarbonitrile

Sodium hydride (50 mg of a 60% dispersion in mineral oil, 1.25 mmol) was suspended in tetrahydrofuran (5 ml) and cooled to 0° C. under an atmosphere of nitrogen. A solution of cis-1-(3-chlorophenyl)-4-hydroxy-cyclohexanecarbonitrile (140 mg, 0.60 mmol) in tetrahydrofuran (2 mL) was added. The mixture was stirred at 0-5° C. for 45 mins. Iodomethane (130 μL, 2.0 mmol) in tetrahydrofuran (1 mL) was then added and the reaction mixture was stirred at room temperature for 2 hours. Further quantities of sodium hydride (50 mg of a 60% dispersion in mineral oil, 1.25 mmol) and iodomethane (130 μL, 2.0 mmol) were added and the reaction stirred for a 1 hour. Water (20 mL) was added cautiously and the reaction mixture was extracted with ethyl acetate (3×15 ml). The combined extracts were dried (Na₂SO₄) and concentrated in vacuo to leave a yellow gum. The gum was purified by flash chromatography (Silica, eluting sequentially with pentane, pentane:diethyl ether 6:1, then 2:1, then 1:1, and finally diethyl ether) to afford the title compound as a colourless oil. ¹Hnmr [400 MHz, CDCl₃, tetramethylsilane as internal standard], δ 1.74-1.88 (4H, m), 2.17-2.29 (4H, m), 3.23 (1H, m), 3.41 (3H, s), 7.28-7.36 (2H, m), 7.40 (1H, m), and 7.46 (1H, br.s).

### C) 1-[cis-1-(3-Chlorophenyl)-4-methoxycyclohexyl]methanamine hydrochloride

A solution of borane-tetrahydrofuran complex (1.4 mL), 1.4 mmol of a 1M solution in tetrahydrofuran) was added to a solution of 1-(3-chlorophenyl)-4-methoxyoxy-cyclohexanecarbonitrile (99 mg, 0.35 mmol) in tetrahydrofuran (5 mL) and the resulting mixture was heated at reflux under a nitrogen atmosphere for 5 hours. The mixture was treated with 6N aq. Hydrochloric acid (5 mL) and methanol (2 mL) and refluxed for 2 hours. The cooled reaction mixture was basified with 1M aq. sodium hydroxide and extracted with dichloromethane (3×10 ml). The combined organic phases were dried (Na₂SO₄) and concentrated in vacuo to leave a colourless oil. The oil was purified on anion-exchange column (SCX cartridge (5 g) eluting sequentially with dichloromethane, dichloromethane:methanol 1:1, dichloromethane:methanol 1:1 with 5% ammonia). Evaporation of the appropriate fractions gave a gum which was further purified by flash chromatography (silica (10 g), eluting with dichloromethane:ethanol:ammonia, 200:8:1 then 100:8:1) to give a colourless oil. The oil was dissolved in methanol (2 mL), treated with 1M hydrochloric acid (2 mL) and concentrated in vacuo to afford the title compound as a white solid.

MS (ES⁺): 254, 256 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 5.97 min.

### Example B4

### 1-[cis-1-(3-Chlorophenyl)-4-(3-phenylpropoxy)cyclohexyl]methanamine hydrochloride

The title compound was prepared analogously as described in Example B3 using (3-bromopropyl)-benzene and sodium iodide instead of iodomethane.

MS (ES⁺): 358, 360 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 8.70 min.

### Example B5

### 1-[cis-4-(Benzyloxy)-1-(3-chlorophenyl)cyclohexyl]methanamine hydrochloride

The title compound was prepared analogously as described in Example B3 using benzyl bromide instead of iodomethane.

MS (ES⁺): 330, 332 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 7.64 min.

### Example B6

### A mixture of 1-[cis-4-methoxy-1-(3-methylphenyl)cyclohexyl]methanamine hydrochloride and 1-[trans-4-methoxy-1-(3-methylphenyl)cyclohexyl]methanamine hydrochloride

This compound was prepared by adaptation of the routes shown in Schemes A and B. The title compounds were prepared analogously as described in Examples A1 and B3 using (meta-tolyl)-acetonitrile instead of 2,5-difluorobenzyl cyanide. The title compounds were obtained as a mixture of diastereoisomers.

MS (ES⁺): 234 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 4.50 and 5.45 min.

### Example B7

### 1-[trans-1-(3-Chlorophenyl)-4-methoxycyclohexyl]methanamine hydrochloride

The title compound was prepared by adaptation of the route depicted in Scheme B.

### A) Isonicotinic acid [trans-4-(3-chlorophenyl)-4-cyano-cyclohexyl]ester

Diethylazodicarboxylate (270 μL) was added to a stirred suspension of cis-1-(3-chlorophenyl)-4-hydroxy-cyclohexanecarbonitrile (400 mg, 1.70 mmol), isonicotinic acid (935 mg, 7.59 mmol) and triphenylphosphine (2.2 g, 8.37 mmol) in toluene (15 mL) under nitrogen and stirring was continued for 18 hours. The reaction mixture was partitioned between sodium bicarbonate (8%, 20 mL) and ethyl acetate (3×10 mL). The combined organic phases were washed with sodium bicarbonate (8%, 20 ml) and water, dried (Na₂SO₄) and concentrated in vacuo to leave a colourless oil. The oil was purified by ion exchange chromatography (SCX cartridge (50 g) eluting sequentially with dichloromethane, dichloromethane:methanol 1:1, and dichloromethane:methanol 1:1 with 5% ammonia) and then by flash chromatography (silica, (20 g) eluting with dichloromethane:ethanol:ammonia, 400:8:1 to 200:8:1) to give an oil. Final purification (silica (10 g) eluting sequentially with pentane, pentane:diethyl ether 9:1, pentane:diethyl ether 4:1 and pentane:diethyl ether 1:1) gave the title compound as a colourless oil.

MS (ES⁺): 341 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 3.70 min.

### B) trans-1-(3-Chlorophenyl)-4-hydroxy-cyclohexanecarbonitrile

A mixture of isonicotinic acid [trans-4-(3-chlorophenyl)-4-cyano-cyclohexyl]ester (254 mg, 0.70 mmol) and 1M aq. lithium hydroxide (3 mL) in tetrahydrofuran (3 mL) was stirred at room temperature for 18 hours. The reaction mixture was diluted with water (20 mL), extracted with ethyl acetate (2×20 mL) and the extracts were washed with 2M aq. sodium carbonate (20 mL) and brine (10 ml). After drying (Na₂SO₄) and concentrating in vacuo, the title compound was obtained as a colourless oil.

MS (ES⁺): 236 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 3.17 min.

### C) 1-[trans-1-(3-Chlorophenyl)-4-methoxy-cyclohexyl]methanamine hydrochloride

The title compound was prepared analogously as described in Example B3 using trans-1-(3-chlorophenyl)-4-hydroxy-cyclohexanecarbonitrile instead of cis-1-(3-chlorophenyl)-4-hydroxy-cyclohexanecarbonitrile.

MS (ES⁺): 254, 256 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 5.12 min.

### Example B8

### 1-[cis-4-Methoxy-1-(2,4,5-trifluorophenyl)cyclohexyl]methanamine hydrochloride

The title compound was prepared by adaptation of the route depicted in Scheme B.

### A) 4-Cyano-4-(2,4,5-trifluorophenyl)-heiptanedioic acid dimethyl ester

A solution of Triton B (2.7 mL, 5.9 mmol of a 40% solution in methanol) in t-butanol (2 mL) was added in one portion to a heated (80° C.) solution of the 2,4,5-trifluorophenyl-acetonitrile (3.0 g, 17.54 mmol) and methyl acrylate (6.3 mL, 70.0 mmol) in t-butanol (6 mL) and the resulting mixture was heated at reflux for 5 h. The reaction mixture was partitioned between 1N hydrochloric acid (40 mL) and diethyl ether (2×30 ml) and the organic phases were washed with brine (20 mL) and blown down. The residue was purified by flash chromatography (silica (50 g), eluting sequentially with pentane, pentane:diethyl ether 9:1, pentane:diethyl ether 3:1 and pentane:diethyl ether 1:1) to give the title compound as a colourless oil.

T_{R} [HPLC, Phenomenex Luna 3 micron C18; 5-95% CH₃CN+0.1% Formic acid/H₂O+0.1% Formic acid for 5 min, flow 2.0 ml/min]: 3.46 min.

### B) 5-Cyano-2-oxo-5-(2,4,5-trifluorophenyl)-cyclohexanecarboxylic acid methyl ester

4-Cyano-4-(2,4,5-trifluorophenyl)-heptanedioic acid dimethyl ester (2.65 g, 7.7 mmol), potassium tert butoxide (1.73 g, 15.4 mmol) and 1,2,4,5-tetrafluorobenzene (1.72 mL, 15.4 mmol) were suspended in dry tetrahydrofuran (50 mL) and the mixture was heated at reflux overnight under an atmosphere of nitrogen. After cooling to room temperature, glacial acetic acid (2.21 mL) in water (30 mL) was added to the reaction mixture which was extracted with diethyl ether (2×30 mL). The organic phases were washed with 1M aq. sodium carbonate (2×30 mL), water (2×30 mL) and brine (2×30 mL), dried (MgSO₄), and concentrated to give an amber coloured gum. The gum was purified by chromatography (silica (50 g), eluting with 5% ethyl acetate in cyclohexane) to give the title compound as a white solid.

MS (ES⁺): 312 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 3.74 min.

### C) 4-Oxo-1-(2,4,5-trifluorophenyl)-cyclohexanecarbonitrile

A mixture of 5-cyano-2-oxo-5-(2,4,5-trifluorophenyl)=cyclohexanecarboxylic acid methyl ester (950 mg, 3.1 mmol), 10% aq. sulphuric acid (10 mL) and glacial acetic acid (22 mL) was heated at 110° C. overnight. After cooling to room temperature, the reaction mixture was diluted with water (20 mL) and extracted into ethyl acetate (20 mL). The organic layer was washed with water (2×20 mL), sat. aq. sodium bicarbonate (20 mL) and brine (20 mL), and dried (MgSO₄). Concentration in vacuo afforded the title as a pale yellow solid.

T_{R} [HPLC, Phenomenex Luna 3 micron C18; 5-95% CH₃CN+0.1% Formic acid/H₂O+0.1% Formic acid for 5 min, flow 2.0 ml/min]: 3.17 min.

### D) 1-[cis-4-Methoxy-1-(2,4,5-trifluorophenyl)cyclohexyl]methanamine hydrochloride

The title compound was prepared analogously as described in Example B3 using 4-oxo-1-(2,4,5-trifluorophenyl)-cyclohexanecarbonitrile instead of 1-(3-chlorophenyl)-4-oxo-cyclohexanecarbonitrile.

MS (ES⁺): 274 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 5.90 min.

### Example B9

### C-(4-Methoxy-1-phenyl-cyclohexyl)-methylamine

The title compound was prepared analogously as described in Example B1 using 1-phenyl-4-hydroxy-cyclohexanecarbonitrile instead of 1-(2,5-Difluoro-phenyl)-4-hydroxy-cyclohexanecarbonitrile.

MS (ES⁺): 220 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 4.32 min.

### Example B10

### C-[1-Phenyl-4-(3-phenyl-propoxy)-cyclohexyl]-methylamine

The title compound was prepared analogously as described in Example B9 using (3-Bromo-propyl)-benzene instead of methyliodide.

MS (ES⁺): 324 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 7.15-7.40 min.

### Example B11

### C-(4-Benzyloxy-1-phenyl-cyclohexyl)-methylamine

The title compound was prepared analogously as described in Example B9 Using benzylbromide instead of methyliodide.

MS (ES⁺): 296 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 6.32 min.

### Example B12

### C-[1-(2-Chloro-phenyl)-4-methoxy-cyclohexyl]-methylamine

The title compound was prepared analogously as described in Example A1 and B1 using 2-chlorobenzyl cyanide instead of 2,5-difluorobenzyl cyanide.

MS (ES⁺): 254 [M+H]⁺.

HPLC (YMC, 10 min method, gradient water/ACN 0-100%): 3.95 min.

### Example B13

### C-[1-(4-Chloro-phenyl)-4-methoxy-cyclohexyl]-methylamine

The title compound was prepared analogously as described in Example A1 and B1 using 4-chlorobenzyl cyanide instead of 2,5-difluorobenzyl cyanide.

MS (ES⁺): 254 [M+H]⁺.

HPLC (YMC, 10 min method, gradient water/ACN 0-100%): 3.57 min.

### Example C1

### C-[1,4-trans-1-Phenyl-4-(3-trifluoromethyl-5,6-dihydro-8H-[1,2,4]triazolo[4,3-a]pyrazin-7-yl)-cyclohexyl]-methylamine

This compound was prepared according to Scheme C:

### A) 1,4-trans-1-Phenyl-4-(3-trifluoromethyl-5,6-dihydro-8H-[1,2,4]triazolo[4,3-a]pyrazin-7-yl)-cyclohexanecarbonitrile

To a solution of 4-oxo-1-phenyl-cyclohexanecarbonitrile (100 mg, 0.50 mmol) in 1,2-dichloroethane (1 ml) were successively added 3-trifluoromethyl-5,6,7,8-tetrahydro-[1,2,4]triazolo[4,3-a]pyrazine (106 mg, 0.55 mmol), sodium triacetoxyborohydride (168 mg, 0.75 mmol), and acetic acid (29 μl, 0.50 mmol). The reaction was stirred at RT for 2 hrs before diluted with EtOAc and quenched with water. The resulting mixture was extracted twice with EtOAc, and the combined organic phase was washed once with brine, dried over Na₂SO₄, and evaporated to provide pale yellow solid. Purification by preparative HPLC yielded the title compound (100 mg) along with its stereoisomer 1,4-cis-1-Phenyl-4-(3-trifluoromethyl-5,6-dihydro-8H-[1,2,4]triazolo[4,3-a]pyrazin-7-yl)-cyclohexanecarbonitrile (18 mg), both as white solids.

MS: 376.0 [M+H]⁺

### B) C-[1,4-trans-1-Phenyl-4-(3-trifluoromethyl-5,6-dihydro-8H-[1,2,4]triazolo[4,3-a]pyrazin-7-yl)-cyclohexyl]-methylamine

To a solution of 1,4-trans-1-Phenyl-4-(3-trifluoromethyl-5,6-dihydro-8H-[1,2,4]triazolo[4,3-a]pyrazin-7-yl)-cyclohexanecarbonitrile (45 mg, 0.12 mmol) in dry THF (1 ml) was added LiAlH₄ (9.4 mg, 0.24 mmol), and the reaction was stirred at 50° C. for 3 h. Another 10 mg of LiAlH₄ was added and the stirring continues further at 60° C. for 2 h. After careful quench with sat. aqueous NH₄Cl solution, the mixture was extracted twice with EtOAc, and the combined organic phase was washed with brine, dried over Na₂SO₄, and concentrated to give the title compound (24 mg) as an yellow solid.

MS: 380.2 [M+H]⁺

### Example D1

### 1-{cis-1-(3-Chlorophenyl)-4-[3-(trifluoromethyl)-5,6-dihydro[1,2,4]triazolo[4,3-a]pyrazin-7(8H)-yl]cyclohexyl}methanamine dihydrochloride

This compound was prepared according to Scheme D:

### A) 4-(3-Chloro-phenyl)-4-cyano-heptanedioic acid dimethyl ester

A solution of Triton B (10 mL of a 40% solution in methanol) in t-butanol (10 mL) was added portion to a heated (80° C.) solution of the 3-chlorophenylacetonitrile (11.65 g, 0.077 mol) and methyl acrylate (19 mL, 0.21 mol) in t-butanol (20 ml) at a rate to maintain a controllable reflux. When the addition wa complete, the reaction mixture was heated at reflux for 2 h. After cooling, the reaction mixture was partitioned between 1N hydrochloric acid (70 mL) and diethyl ether (3×30 mL) and then the organic phases were washed with brine (20 mL) and concentrated. The residue was recrystallised from dieth ether:pentane 1:1 to give the title compound as a white solid, m.p. 78.5-80° C.

T_{R} [HPLC, Phenomenex Luna 3 micron C18; 5-95% CH₃CN+0.1% Formic acid/H₂O+0.1%

Formic acid for 5 min, flow 2.0 ml/min]: 3.57 min.

¹Hnmr [400 MHz, CDCl₃, tetramethylsilane as internal standard], δ 2.13 (2H, m) 2.28 (2H, m), 2.38 (2H, m), 2.51 (2H, m), 3.63 (6H, s), 7.28-7.42 (4H, m).

### B) 5-(3-Chlorophenol)-5-cyano-2-oxo-cyclohexanecarboxylic acid methyl ester

Potassium tert-butoxide (4.8 g, 43.0 mmol) was added in one portion to a stirred solution of 4-(3-chloro-phenyl)-4-cyano-heptanedioic acid dimethyl ester (6.23 g, 19.3 mmol) in anhydrous tetrahydrofuran (80 mL). The resulting mixture was stirred at reflux for 5 h. The reaction mixture was cooled (0° C.) and treated with a solution of acetic acid (4.5 mL) in water (30 mL). The mixture was extracted with diethyl ether (70 mL) and the organic phase was washed with aqueous sodium carbonate solution (2N, 80 mL), water (2×40 mL) and brine (20 mL) and then dried (Na₂SO₄). After concentration in vacuo, the title product was obtained as a white solid.

MS (ES⁻): 290 and 292 [M−H]⁻.

T_{R} [HPLC, Phenomenex Luna 3 micron C18; 5-95% CH₃CN+0.1% Formic acid/H₂O+0.1% Formic acid for 5 min, flow 2.0 ml/min]: 3.95 min.

### C) 1-(3-Chlorophenol)-4-oxo-cyclohexanecarbonitrile

A mixture of 5-(3-chlorophenyl)-5-cyano-2-oxo-cyclohexanecarboxylic acid methyl ester (8.0 g, 27.4 mmol) and 10% aqueous sulphuric acid (40 mL) in acetic acid (80 mL) was heated overnight at 110° C. After cooling to room temperature, the reaction mixture was diluted with water (200 mL) and extracted into EtOAc (70 mL×3) The combined organic phases were washed with sodium bicarbonate solution (8%, 3×50 mL), water (2×50 mL) and brine (20 mL), and then dried (Na₂SO₄). After concentration the title compound was obtained as an orange oil.

MS (ES⁺): 234 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 3.32 min.

### D) 8-(3-Chlorophenol)-1,4-dioxa-spiro[4.5]decane-8-carbonitrile

Para-Toluenesulphonic acid (0.37 g, 1.95 mmol) and ethylene glycol (48 mL) were added to a solution of 1-(3-chlorophenyl)-4-oxo-cyclohexanecarbonitrile (22.3 g, 95.4 mmol) in toluene (250 mL) and the mixture was heated at 140-143° C. for 6 hours using a Dean and Stark apparatus to remove excluded water. After cooling to room temperature, the toluene was removed by evaporation to give a pale yellow oil. The oil was dissolved in diethyl ether (300 mL) and the solution washed with water (2×150 mL). The aqueous layers were combined and back extracted with diethyl ether (200 mL). The combined organics were washed with brine (100 mL), dried (MgSO4), and evaporated to give the title product as a pale yellow oil, which solidified on standing to give a colourless wax.

T_{R} [HPLC, Phenomenex Luna 3 micron C18; 5-95% CH₃CN+0.1% Formic acid/H₂O+0.1% Formic acid for 5 min, flow 2.0 ml/min]: 3.78 min.

¹Hnmr [400 MHz, CDCl₃, tetramethylsilane as internal standard], δ 1.87 (2H, m), 2.05-2.20 (6H, m), 3.99 (4H, m), 7.28-7.36 (2H, m), 7.42 (1H, m), and 7.49 (1H, br.s).

### E) C-[8-(3-Chlorophenyl)-1,4-dioxa-spiro[4.5]dec-8-yl]-methylamine

A solution of the 8-(3-chlorophenyl)-1,4-dioxa-spiro[4.5]decane-8-carbonitrile (6.0 g, 21.6 mmol) in tetrahydrofuran (15 mL) was added dropwise to a stirred suspension of lithium aluminium hydride (2.0 g, 52.7 mmol) in tetrahydrofuran (5 mL). The reaction was stirred at room temperature for 1 hour then cautiously quenched with saturated aqueous Rochelle's salt (30 mL) and extracted into ethyl acetate (3×40 mL). The combined organics were washed with water and brine, dried (Na₂SO₄) and concentrated. The residue was purified by flash chromatography (Silica cartridge (25 g) using gradient elution with dichloromethane:ethanol:ammonia from 400:8:1 to 100:8:1) to give a colourless oil. The oil was further purified (SCX cartridge (25 g) eluting with dichloromethane then dichloromethane:methanol 1:1, then dichloromethane:methanol 1:1 with 5% ammonia) to give the title compound as a cream solid.

MS (ES⁺): 282 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 1.93 min.

### F) [8-(3-Chlorophenyl)-1,4-dioxa-spiro[4.5]dec-8-ylmethyl]-carbamic acid tert-butyl ester

Tert-Butyloxycarbonyl anhydride (3.6 g, 16.5 mmol) was added to a stirred solution of C-[8-(3-chlorophenyl)-1,4-dioxa-spiro[4.5]dec-8-yl]-methylamine (3.9 g, 13.8 mmol) and triethylamine (7 mL) in tetrahydrofuran (40 mL) and the mixture was stirred for 18 h. The mixture was partitioned between 1N hydrochloric acid (20 mL) and extracted with ethyl acetate (3×10 mL).

The combined organic phases were washed with water (20 mL) and brine (10 mL), dried (Na₂SO₄), and concentrated in vacuo to give a brown oil. The oil was purified by flash chromatography (silica cartridge (50 g) eluting sequentially with pentane, pentane:diethylether (4:1), pentane:diethylether (1:1) and diethyl ether) to give the title compound as a yellow oil.

MS (ES⁺): 382 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 3.96 min.

### G) [1-(3-Chlorophenyl)-4-oxo-cyclohexylmethyl]-carbamic acid tert-butyl ester

Pyridinium para-toluene sulphonate (1.16 g, 4.62 mmol) was added to a stirred solution of the [8-(3-chlorophenyl)-1,4-dioxa-spiro[4.5]dec-8-ylmethyl]-carbamic acid tert-butyl ester (11.0 g, 23.0 mmol) in a mixture of acetone (120 mL) and water (12 mL). The resulting solution was then heated to gentle reflux for 16 h. A further aliquot of pyridinium para-toluene sulphonate (1.16 g, 4.62 mmol) was added and the mixture was heated for an additional 20 h. After cooling, the volatiles were evaporated to give a yellow solid, which was purified by column chromatography (Silica cartridge (330 g), using gradient elution with 10-30% ethyl acetate in cyclohexane) to give the title compound as a white solid.

MS (ES⁺): 338 and 340[M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 3.62 min.

### H) ({cis-1-(3-Chlorophenyl)-4-[3-(trifluoromethyl)-5,6-dihydro[1,2,4]triazolo[4,3-a]pyrazin-7(8H)-yl]cyclohexyl}methyl)-carbamic acid tert-butyl ester and ({trans-1-(3-chlorophenyl)-4-[3-(trifluoromethyl)-5,6-dihydro[1,2,4]triazolo[4,3-a]pyrazin-7(8H)-yl]cyclohexyl}methyl)-carbamic acid tert-butyl ester

Sodium triacetoxyborohydride (316 mg, 1.49 mmol) was added to a solution of [1-(3-chlorophenyl)-4-oxo-cyclohexylmethyl]-carbamic acid tert-butyl ester (360 mg, 1.07 mmol) and 3-trifluoromethyl-5,6,7,8-tetrahydro-[1,2,4]triazolo[4,3-a]pyrazine (286.4 mg, 1.49 mmol) in 1,2-dichloroethane and the mixture was stirred at room temperature for 24 h. The reaction was quenched with water and the product was extracted with ethyl acetate. The organic extracts were washed with brine, dried and concentrated in vacuo to give a yellow oil. The oil was purified by flash chromatography (silica, eluting with 1:33:66 2M ammonia in methanol:ethyl acetate:cyclohexane) to afford the individual title compounds as white solids.

Cis diastereoisomer:

MS (ES⁺): 514 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 3.70 min.

Trans diastereoisomer:

MS (ES⁺): 514 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 3.57 min.

### I) 1-{cis-1-[3-chlorophenyl)-4-[3-(trifluoromethyl)-5,6-dihydro[1,2,4]triazolo[4,3-a]pyrazin-7(8H)-yl]cyclohexyl}methanamine dihydrochloride

Trifluoroacetic acid (1 mL) was added to a solution of ({cis-1-(3-chlorophenyl)-4-[3-(trifluoromethyl)-5,6-dihydro[1,2,4]triazolo[4,3-a]pyrazin-7(8H)-yl]cyclohexyl}methyl)-carbamic acid tert-butyl ester (93 mg, 0.181 mmol) in dichloromethane (10 mL) and the reaction stirred at room temperature for 90 mins. The reaction mixture was concentrated in vacuo and the residue was purified (SCX cartridge eluting sequentially with dichloromethane, methanol and 0.5M ammonia in methanol). Fractions containing the product were concentrated in vacuo to give the free base of the title compound, which was dissolved in dichloromethane and treated with excess 1M hydrogen chloride in methanol. Removal of the volatiles gave the title compound as a white solid.

MS (ES⁺): 414 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 5.96 min.

### Example D2

### 1-{trans-1-(3-Chlorophenyl)-4-[3-trifluoromethyl)-5,6-dihydro[1,2,4]triazolo[4,3-a]pyrazin-7(8H)-yl]cyclohexyl}methanamine dihydrochloride

The title compound was prepared analogously as described in Example D1, step I from ({trans-1-(3-chlorophenyl)-4-[3-(trifluoromethyl)-5,6-dihydro[1,2,4]triazolo[4,3-a]pyrazin-7(8H)-yl]cyclohexyl}methyl)-carbamic acid tert-butyl ester.

MS (ES⁺): 414 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 5.36 min.

### Example D3

### 1-{cis-[4-(4-benzylpiperidin-1-yl)-1-phenylcyclohexyl]}methanamine dihydrochloride and 1-{trans-[4-(4-benzylpiperidin-1-yl)-1-phenylcyclohexyl]}methanamine dihydrochloride

The title compounds were prepared analogously as described in Example D1 using phenylacetonitrile instead of 3-chlorophenylacetonitrile and 4-benzylpiperidine instead of 3-trifluoromethyl-5,6,7,8-tetrahydro-[1,2,4]triazolo[4,3-a]pyrazine, and were isolated as a mixture of diastereoisomers.

MS (ES⁺): 363 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 5.12 min.

### Example D4

### 1-{cis-[4-(4-Benzylpiperazin-1-yl)-1-phenylcyclohexyl]}methanamine dihydrochloride and 1-{trans-[4-(4-benzylpiperazin-1-yl)-1-phenylcyclohexyl]}methanamine dihydrochloride

The title compounds were prepared analogously as described in Example D1 using phenylacetonitrile instead of 3-chlorophenylacetonitrile and 1-benzylpiperazine instead of 3-trifluoromethyl-5,6,7,8-tetrahydro-[1,2,4]triazolo[4,3-a]pyrazine, and were isolated as a mixture of diastereoisomers.

MS (ES⁺): 364 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 3.59 min.

### Example D5

### 1-{cis-[1-Phenyl-4-(4-phenylpiperazin-1-yl)cyclohexyl]}methanamine dihydrochloride and 1-{trans-[1-phenyl-4-(4-phenylpiperazin-1-yl)cyclohexyl]}methanamine dihydrochloride

The title compounds were prepared analogously as described in Example D1 using phenylacetonitrile instead of 3-chlorophenylacetonitrile and 1-phenylpiperazine instead of 3-trifluoromethyl-5,6,7,8-tetrahydro-[1,2,4]triazolo[4,3-a]pyrazine, and were isolated as a mixture of diastereoisomers.

MS (ES⁺): 350 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 4.32 and 4.43 min.

### Example D6

### 1-{cis-[4-(4-tert-Butylpiperidin-1-yl)-1-phenylcyclohexyl]}methanamine and 1-{trans-[4-(4-tert-butylpiperidin-1-yl)-1-phenylcyclohexyl]}methanamine

The title compounds were prepared analogously as described in Example D1 using phenylacetonitrile instead of 3-chlorophenylacetonitrile and 4-tert-butylpiperidine instead of 3-trifluoromethyl-5,6,7,8-tetrahydro-[1,2,4]triazolo[4,3-a]pyrazine, and were isolated as a mixture of diastereoisomers.

MS (ES⁺): 329 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 4.95 and 5.10 min.

### Example D7

### 1-{cis-[4-(4-Methylpiperidin-1-yl)-1-phenylcyclohexyl]}ethanamine dihydrochloride and 1-{trans-[4-(4-methylpiperidin-1-yl)-1-phenylcyclohexyl]}methanamine dihydrochloride

The title compounds were prepared analogously as described in Example D1 using phenylacetonitrile instead of 3-chlorophenylacetonitrile and 4-tert-butylpiperidine instead of 3-trifluoromethyl-5,6,7,8-tetrahydro-[1,2,4]triazolo[4,3-a]pyrazine, and were isolated as a mixture of diastereoisomers.

MS (ES⁺): 287 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 1.25 and 3.57 min.

### Example D8

### 1-{cis-[4-(4-Benzylpiperidin-1-yl)-1-(3-chlorophenyl)cyclohexyl]}methanamine dihydrochloride and 1-{trans-[4-(4-benzylpiperidin-1-yl)-1-(3-chlorophenyl)cyclohexyl]}methanamine dihydrochloride

The title compounds were prepared analogously as described in Example D1 using 4-benzylpiperidine instead of 3-trifluoromethyl-5,6,7,8-tetrahydro-[1,2,4]triazolo[4,3-a]pyrazine, and were isolated as a mixture of diastereoisomers.

MS (ES⁺): 397, 399 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 4.75 and 4.87 min.

### Example 09

### 1′-{cis-[4-(Aminomethyl)-4-(3-chlorophenyl)cyclohexyl]}-1,4% bipiperidin-2-one dihydrochloride and 1′-{trans-[4-(aminomethyl)-4-(3-chlorophenyl)cyclohexyl]}-1,4′-bipiperidin-2-one dihydrochloride

The title compounds were prepared analogously as described in Example D1 using [1,4′]bipiperidinyl-2-one instead of 3-trifluoromethyl-5,6,7,8-tetrahydro-[1,2,4]triazolo[4,3-a]pyrazine, and were isolated as a mixture of diastereoisomers.

MS (ES⁺): 404, 406 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 3.31 min.

### Example D10

### 1-{1-[cis-[4-(Aminomethyl)-4-(3-chlorophenyl)cyclohexyl]piperidin-4-yl]}pyrrolidin-2-one dihydrochloride and 1-{1-[trans-[4-(aminomethyl)-4-(3-chlorophenyl)cyclohexyl]piperidin-4-yl]}pyrrolidin-2-one dihydrochloride

The title compounds were prepared analogously as described in Example D1 using 1-piperidin-4-yl-pyrrolidin-2-one instead of 3-trifluoromethyl-5,6,7,8-tetrahydro-[1,2,4]triazolo[4,3-a]pyrazine, and were isolated as a mixture of diastereoisomers.

MS (ES⁺): 390, 392 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 3.24 min.

### Example D11

### 1-[cis-{1-(3-Chlorophenyl)-4-[4(1H-imidazol-1-yl)piperidin-1-yl]cyclohexyl}]methanamine dihydrochloride and 1-[trans-{1-(3-chlorophenyl)-4-[4-(1H-imidazol-1-yl)piperidin-1-yl]cyclohexyl}]methanamine dihydrochloride

The title compounds were prepared analogously as described in Example D1 using 4-imidazol-1-yl-piperidine instead of 3-trifluoromethyl-5,6,7,8-tetrahydro-[1,2,4]triazolo[4,3-a]pyrazine, and were isolated as a mixture of diastereoisomers.

MS (ES⁺): 373, 375 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 0.68 min.

### Example D12

### 1-{cis-[4-(Aminomethyl)-4-(3-chlorophenyl)cyclohexyl]}piperidine-3-carboxamide dihydrochloride and 1-{trans-[4-(aminomethyl)-4-(3-chlorophenyl)cyclohexyl]}piperidine-3-carboxamide dihydrochloride

The title compounds were prepared analogously as described in Example D1 using piperidine-3-carboxamide instead of 3-trifluoromethyl-5,6,7,8-tetrahydro-[1,2,4]triazolo[4,3-a]pyrazine, and were isolated as a mixture of diastereoisomers.

MS (ES⁺): 350, 352 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 1.17 min.

### Example D13

### 1-{cis-[1-(3-Chlorophenyl)-4-[4-(2-phenylethyl)piperazin-1-yl]cyclohexyl]}methanamine hydrochloride and 1-{trans-[1-(3-chlorophenyl)-4-[4-(2-phenylethyl)piperazin-1-yl]cyclohexyl]}methanamine hydrochloride

The title compounds were prepared analogously as described in Example D1 using 1-phenethyl-piperazine instead of 3-trifluoromethyl-5,6,7,8-tetrahydro-[1,2,4]triazolo[4,3-a]pyrazine, and were isolated as a mixture of diastereoisomers.

MS (ES⁺): 412, 414 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 3.91 and 4.41 min.

### Example D14

### 1-{cis-[1-(3-Chlorophenyl)-4-[4-(2-furoyl)piperazin-1-yl]cyclohexyl]}methanamine hydrochloride and 1-{trans-[1-(3-chlorophenyl)-4-[4-(2-furoyl)piperazin-1-yl]cyclohexyl]}methanamine hydrochloride

The title compounds were prepared analogously as described in Example D1 using 1-(2-furoyl)-piperazine instead of 3-trifluoromethyl-5,6,7,8-tetrahydro-[1,2,4]triazolo[4,3-a]pyrazine, and were isolated as a mixture of diastereoisomers.

MS (ES⁺): 402, 404 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 2.88 and 3.53 min.

### Example D15

### 1-{cis-[1-(3-Chlorophenyl)-4-(4-pyrimidin-2-ylpiperazin-1-yl)cyclohexyl]}methanamine dihydrochloride and 1-{trans-[1-(3-chlorophenyl)-4-(4-pyrimidin-2-ylpiperazin-1-yl)cyclohexyl]}methanamine dihydrochloride

The title compounds were prepared analogously as described in Example D1 using 2-piperazin-1-yl-pyrimidine instead of 3-trifluoromethyl-5,6,7,8-tetrahydro-[1,2,4]triazolo[4,3-a]pyrazine, and were isolated as a mixture of diastereoisomers.

MS (ES⁺): 386, 388 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 3.60 and 3.84 min.

### Example D16

### 1-{cis-[1-(3-Chlorophenyl)-4-(4-pyrazin-2-ylpiperazin-1-yl)cyclohexyl]}methanamine dihydrochloride and 1-{trans-[1-(3-chlorophenyl)-4-(4-pyrazin-2-ylpiperazin-1-yl)cyclohexyl]}methanamine dihydrochloride

The title compounds were prepared analogously as described in Example D1 using 3,4,5,6-tetrahydro-2H-[1,2]bipyrazine instead of 3-trifluoromethyl-5,6,7,8-tetrahydro-[1,2,4]triazolo[4,3-a]pyrazine, and were isolated as a mixture of diastereoisomers.

MS (ES⁺): 386, 388 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 3.28 and 3.71 min.

### Example D17

### 1-{cis-(1-(3-Chlorophenyl)-4-{4-[2-fluoro-4-(methylsulfonyl)phenyl]piperazin-1-yl}cyclohexyl)}methanamine dihydrochloride and 1-{trans-(1-(3-chlorophenyl)-4-{4-[2-fluoro-4-(methylsulfonyl)phenyl]piperazin-1-yl}cyclohexyl)}methanamine dihydrochloride

The title compounds were prepared analogously as described in Example D1 using 1-(2-fluoro-4-methanesulphonyl-phenyl)-piperazine instead of 3-trifluoromethyl-5,6,7,8-tetrahydro-[1,2,4]triazolo[4,3-a]pyrazine, and were isolated as a mixture of diastereoisomers.

MS (ES⁺): 480, 482 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 4.19 and 4.46 min.

### Example D18

### 1-{cis-(1-[4-(Aminomethyl)-4-(3-chlorophenyl)cyclohexyl]piperidin-4-yl)}-1,3-dihydro-2H-benzimidazol-2-one dihydrochloride and 1-{trans-(1-[4-(aminomethyl)-4-(3-chlorophenyl)cyclohexyl]piperidin-4-yl)}-1,3-dihydro-2H-benzimidazol-2-one dihydrochloride

The title compounds were prepared analogously as described in Example D1 using 1-piperidin-4-yl-1,3-dihydro-benzoimidazol-2-one instead of 3-trifluoromethyl-5,6,7,8-tetrahydro-[1,2,4]triazolo[4,3-a]pyrazine, and were isolated as a mixture of diastereoisomers.

MS (ES⁺): 439, 441 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 4.26 min.

### Example D19

### 1-{cis-[1-(3-Chlorophenyl)-4-[4-(2-oxo-2-pyrrolidin-1-ylethyl)piperazin-1-yl]cyclohexyl]}methanamine dihydrochloride and 1-{trans-[1-(3-chlorophenyl)-4-[4-(2-oxo-2-pyrrolidin-1-ylethyl)piperazin-1-yl]cyclohexyl]}methanamine dihydrochloride

The title compounds were prepared analogously as described in Example D1 using 2-piperazin-1-yl-1-pyrrolidin-1-yl-ethanone instead of 3-trifluoromethyl-5,6,7,8-tetrahydro-[1,2,4]triazolo[4,3-a]pyrazine, and were isolated as a mixture of diastereoisomers.

MS (ES⁺): 418, 420 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 3.14 and 3.47 min.

### Example D20

### 1-{cis-[1-(3-Chlorophenyl)-4-(3,4-dihydroisoquinolin-2(1H)-yl)}cyclohexyl]methanamine hydrochloride and 1-{trans-[1-(3-chlorophenyl)-4-(3,4-dihydroisoquinolin-2(1H)-yl)}cyclohexyl]methanamine hydrochloride

The title compounds were prepared analogously as described in Example D1 using 1,2,3,4-tetrahydroisoquinoline instead of 3-trifluoromethyl-5,6,7,8-tetrahydro-[1,2,4]triazolo[4,3-a]pyrazine, and were isolated as a mixture of diastereoisomers.

MS (ES⁺): 355, 357 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 3.89 and 4.07 min.

### Example D21

### 1-{cis-(1-(3-Chlorophenyl)-4-{4-[4-(trifluoromethyl)pyrimidin-2-yl]piperazin-1-yl}cyclohexyl)}methanamine hydrochloride and 1-{trans-(1-(3-chlorophenyl)-4-{4-[4-(trifluoromethyl)pyrimidin-2-yl]piperazin-1-yl}cyclohexyl)}methanamine hydrochloride

The title compounds were prepared analogously as described in Example D1 using 2-piperazin-1-yl-4-trifluoromethyl-piperidine instead of 3-trifluoromethyl-5,6,7,8-tetrahydro-[1,2,4]triazolo[4,3-a]pyrazine, and were isolated as a mixture of diastereoisomers.

MS (ES⁺): 454, 456 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 5.46 min.

### Example D22

### 1-{cis-[4-(Aminomethyl)-4-(3-chlorophenyl)cyclohexyl]}-1,4-diazepan-5-one hydrochloride and 1-{trans-[4-(aminomethyl)-4-(3-chlorophenyl)cyclohexyl]}-1,4-diazepan-5-one hydrochloride

The title compounds were prepared analogously as described in Example D1 using [1,4]diazepan-5-one instead of 3-trifluoromethyl-5,6,7,8-tetrahydro-[1,2,4]triazolo[4,3-a]pyrazine, and were isolated as a mixture of diastereoisomers.

MS (ES⁺): 336, 338 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 1.11 and 1.16 min.

### Example D23

### 1-{cis-(1-(3-Chlorophenyl)-4-{4-[4-fluoro-2-(methylsulfonyl)phenyl]piperazin-1-yl}cyclohexyl)}methanamine hydrochloride and 1-{trans-(1-(3-chlorophenyl)-4-{4-[4-fluoro-2-(methylsulfonyl)phenyl]piperazin-1-yl}cyclohexyl)}methanamine hydrochloride

The title compounds were prepared analogously as described in Example D1 using 1-(4-fluoro-2-methanesulphonyl-phenyl)-piperazine instead of 3-trifluoromethyl-5,6,7,8-tetrahydro-[1,2,4]triazolo[4,3-a]pyrazine, and were isolated as a mixture of diastereoisomers.

MS (ES⁺): 480, 482 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 4.57 and 4.75 min.

### Example D24

### 1-{cis-[1-(3-Chlorophenyl)-4-[4-(1H-1,2,4-triazol-1-yl)piperidin-1-yl]cyclohexyl]}methanamine dihydrochloride and 1-{trans-[1-(3-chlorophenyl)-4-[4-(1H-1,2,4-triazol-1-yl)piperidin-1-yl]cyclohexyl]}methanamine dihydrochloride

The title compounds were prepared analogously as described in Example D1 using 4-[1,2,4]triazol-1-yl-piperidine instead of 3-trifluoromethyl-5,6,7,8-tetrahydro-[1,2,4]triazolo[4,3-a]pyrazine, and were isolated as a mixture of diastereoisomers.

MS (ES⁺): 374, 376 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 2.81 min.

### Example D25

### 1-{cis-[1-(3-Chlorophenyl)-4-(1,3-dihydro-2H-isoindol-2-yl)cyclohexyl]}methanamine dihydrochloride and 1-{trans-[1-(3-chlorophenyl)-4-(1,3-dihydro-2H-isoindol-2-yl)cyclohexyl]}methanamine dihydrochloride

The title compounds were prepared analogously as described in Example D1 using 2,3-dihydro-1H-isoindole instead of 3-trifluoromethyl-5,6,7,8-tetrahydro-[1,2,4]triazolo[4,3-a]pyrazine, and were isolated as a mixture of diastereoisomers.

MS (ES⁺): 341, 343 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 3.86 min.

### Example D26

### 4-{cis-[4-(Aminomethyl)-4-(3-chlorophenyl)cyclohexyl]}piperazin-2-one

The title compound was prepared analogously as described in Example D1 using piperazine-2-one instead of 3-trifluoromethyl-5,6,7,8-tetrahydro-[1,2,4]triazolo[4,3-a]pyrazine.

MS (ES⁺): 322, 324 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 1.13 min.

### Example D27

### 4-{trans-[4-(Aminomethyl)-4-(3-chlorophenyl)cyclohexyl]}piperazin-2-one

The title compound was prepared analogously as described in Example D1 using piperazine-2-one instead of 3-trifluoromethyl-5,6,7,8-tetrahydro-[1,2,4]triazolo[4,3-a]pyrazine.

MS (ES⁺): 322, 324 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 1.18 min.

### Example D28

### 1-(cis-4-Morpholin-4-yl-1-phenylcyclohexyl)methanamine dihydrochloride and 1-(trans-4-morpholin-4-yl-1-phenylcyclohexyl)methanamine dihydrochloride

The title compound was prepared analogously as described in Example D1 using 1-phenyl-4-oxo-cyclohexanecarbonitrile instead of 1-(3-chlorophenyl)-4-oxo-cyclohexanecarbonitrile and morpholine instead of 3-trifluoromethyl-5,6,7,8-tetrahydro-[1,2,4]triazolo[4,3-a]pyrazine, and were isolated as a mixture of diastereoisomers.

MS (ES⁺): 275 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 1.13 min.

### Example D29

### 1-[cis-4-(4-Methylpiperazin-1-yl)-1-phenylcyclohexyl]methanamine dihydrochloride and 1-[trans-4-(4-methylpiperazin-1-yl)-1-phenylcyclohexyl]methanamine dihydrochloride

The title compound was prepared analogously as described in Example D1 using 1-phenyl-4-oxo-cyclohexanecarbonitrile instead of 1-(3-chlorophenyl)-4-oxo-cyclohexanecarbonitrile and 1-methyl-piperazine instead of 3-trifluoromethyl-5,6,7,8-tetrahydro-[1,2,4]triazolo[4,3-a]pyrazine, and were isolated as a mixture of diastereoisomers.

MS (ES⁺): 288 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 1.14 and 1.36 min.

### Example D30

### cis-4-(Aminomethyl)-N-cyclohexyl-4-phenylcyclohexanamine dihydrochloride and trans-4-(aminomethyl)-N-cyclohexyl-4-phenylcyclohexanamine dihydrochloride

The title compound was prepared analogously as described in Example D1 using 1-phenyl-4-oxo-cyclohexanecarbonitrile instead of 1-(3-chlorophenyl)-4-oxo-cyclohexanecarbonitrile and cyclohexylamine instead of 3-trifluoromethyl-5,6,7,8-tetrahydro-[1,2,4]triazolo[4,3-a]pyrazine, and were isolated as a mixture of diastereoisomers.

MS (ES⁺): 287 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 2.99 and 4.39 min.

### Example D31

### 1-(cis-4-Azepan-1-yl-1-phenylcyclohexyl)methanamine dihydrochloride and 1-(trans-4-azepan-1-yl-1-phenylcyclohexyl)methanamine dihydrochloride

The title compound was prepared analogously as described in Example D1 using 1-phenyl-4-oxo-cyclohexanecarbonitrile instead of 1-(3-chlorophenyl)-4-oxo-cyclohexanecarbonitrile and azepane instead of 3-trifluoromethyl-5,6,7,8-tetrahydro-[1,2,4]triazolo[4,3-a]pyrazine, and were isolated as a mixture of diastereoisomers.

MS (ES⁺): 287 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 3.36 min.

### Example D32

### Benzyl 4-[cis-4-(aminomethyl)-4-(3-chlorophenyl)cyclohexyl]piperazine-1-carboxylate hydrochloride and benzyl 4-[trans-4-(aminomethyl)-4-(3-chlorophenyl)cyclohexyl]piperazine-1-carboxylate hydrochloride

The title compound was prepared analogously as described in Example D1 using piperazine-1-carboxylic acid benzyl ester instead of 3-trifluoromethyl-5,6,7,8-tetrahydro-[1,2,4]triazolo[4,3-a]pyrazine, and were isolated as a mixture of diastereoisomers.

MS (ES⁺): 442, 444 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 5.40 min.

### Example D33

### cis-4-(Aminomethyl)-4-(3-chlorophenol)-N-[(1,5-dimethyl-1H-pyrazol-3-yl)methyl]cyclohexanamine dihydrochloride and trans-4-(aminomethyl)-4-(3-chlorophenyl)-N-[(1,5-dimethyl-1H-pyrazol-3-yl)methyl]cyclohexanamine dihydrochloride

The title compound was prepared analogously as described in Example D1 using C-(1,5-dimethyl-1H-pyrazol-3-yl)-methylamine instead of 3-trifluoromethyl-5,6,7,8-tetrahydro-[1,2,4]triazolo[4,3-a]pyrazine, and were isolated as a mixture of diastereoisomers.

MS (ES⁺): 347, 349 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 1.18 min.

### Example D34

### 1-[cis-4-[3-(Trifluoromethyl)-5,6-dihydro[1,2,4]triazolo[4,3-a]pyrazin-7(8H)-yl]-1-(2,4,5-trifluorophenyl)cyclohexyl]methanamine hydrochloride and 1-[trans-4-[3-(trifluoromethyl)-5,6-dihydro[1,2,4]triazolo[4,3-a]pyrazin-7(8H)-yl]-1-(2,4,5-trifluorophenyl)cyclohexyl]methanamine hydrochloride

The title compound was prepared analogously as described in Example D1 using 4-oxo-1-(2,4,5-trifluorophenyl)-cyclohexanecarbonitrile instead of 1-(3-chlorophenyl)-4-oxo-cyclohexanecarbonitrile, and were isolated as a mixture of diastereoisomers.

MS (ES⁺): 434, 436 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 5.40 and 5.93 min.

### Example D35

### 1-(3-{[cis-4-(Aminomethyl)-4-(3-chlorophenyl)cyclohexyl]amino}propyl)pyrrolidine-2,5-dione hydrochloride

The title compound was prepared analogously as described in Example D1 using 1-(3-amino-propyl)-pyrrolidine-2,5-dione instead of 3-trifluoromethyl-5,6,7,8-tetrahydro-[1,2,4]triazolo[4,3-a]pyrazine.

MS (ES⁺): 323, 325 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 3.11 min.

### Example D36

### 1-(3-{[trans-4-(Aminomethyl)-4-(3-chlorophenyl)cyclohexyl]amino}propyl)pyrrolidine-2,5-dione hydrochloride

The title compound was prepared analogously as described in Example D1 and D2 using 1-(3-amino-propyl)-pyrrolidine-2,5-dione instead of 3-trifluoromethyl-5,6,7,8-tetrahydro-[1,2,4]triazolo[4,3-a]pyrazine.

MS (ES⁺): 378, 380 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 3.89 min.

### Example D37

### N-[cis-4-(Aminomethyl)-4-(3-chlorophenyl)cyclohexyl]tetrahydro-2H-pyran-4-amine hydrochloride and N-[trans-4-(aminomethyl)-4-(3-chlorophenyl)cyclohexyl]tetrahydro-2H-pyran-4-amine hydrochloride

The title compounds were prepared analogously as described in Example D1 using tetrahydropyran-4-ylamine instead of 3-trifluoromethyl-5,6,7,8-tetrahydro-[1,2,4]triazolo[4,3-a]pyrazine and were isolated as a mixture of diastereoisomers.

MS (ES⁺): 378, 380 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 3.89 min.

### Example D38

### cis-4-(Aminomethyl)-4-(3-chlorophenol)-N-[(1-methyl-1H-imidazol-4-yl)methyl]cyclohexanamine hydrochloride

The title compound was prepared analogously as described in Example D1 using C-(1-methyl-1H-imidazol-4-yl)-methylamine instead of 3-trifluoromethyl-5,6,7,8-tetrahydro-[1,2,4]triazolo[4,3-a]pyrazine.

MS (ES⁺): 333, 335 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 1.19 min.

### Example D39

### trans-4-(Aminomethyl)-4-(3-chlorophenyl)-N-[(1-methyl-1H-imidazol-4-yl)methyl]cyclohexanamine hydrochloride

The title compound was prepared analogously as described in Example D1 and D2 using C-(1-methyl-1H-imidazol-4-yl)-methylamine instead of 3-trifluoromethyl-5,6,7,8-tetrahydro-[1,2,4]triazolo[4,3-a]pyrazine.

MS (ES⁺): 333, 335 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 1.02 min.

### Example D40

### cis-4-(Aminomethyl)-4-(3-chlorophenyl)-N-(2-phenylethyl)cyclohexanamine hydrochloride and trans-4-(aminomethyl)-4-(3-chlorophenyl)-N-(2-phenylethyncyclohexanamine hydrochloride

The title compounds were prepared analogously as described in Example D1 using 2-phenylethylamine instead of 3-trifluoromethyl-5,6,7,8-tetrahydro-[1,2,4]triazolo[4,3-a]pyrazine and were isolated as a mixture of diastereoisomers.

MS (ES⁺): 343, 345 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 4.22, 4.88 min.

### Example D41

### 3-[cis-[4-(Aminomethyl)-4-(3-chlorophenyl)cyclohexyl](methyl)amino]propanenitrile hydrochloride and 3-[trans-[4-(aminomethyl)-4-(3-chlorophenyl)cyclohexyl](methyl)amino]propanenitrile hydrochloride

The title compounds were prepared analogously as described in Example D1 using 3-methylamino-propionitrile instead of 3-trifluoromethyl-5,6,7,8-tetrahydro-[1,2,4]triazolo[4,3-a]pyrazine and were isolated as a mixture of diastereoisomers.

MS (ES⁺): 306, 308 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 1.13 min.

### Example D42

### cis-4-(Aminomethyl)-N-benzyl-4(3-chlorophenyl)cyclohexanamine hydrochloride and trans-4-(aminomethyl)-N-benzyl-4-(3-chlorophenyl)cyclohexanamine hydrochloride

The title compounds were prepared analogously as described in Example D1 using benzylamine instead of 3-trifluoromethyl-5,6,7,8-tetrahydro-[1,2,4]triazolo[4,3-a]pyrazine and were isolated as a mixture of diastereoisomers.

MS (ES⁺): 329, 331 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 3.54, 3.61 min.

### Example D43

### cis-4-(Aminomethyl)-4-(3-chlorophenyl)-N-(cyclopropylmethyl)cyclohexanamine hydrochloride and trans-4-(aminomethyl)-4-(3-chlorophenyl)-N-(cyclopropylmethyl)cyclohexanamine hydrochloride

The title compounds were prepared analogously as described in Example D1 using C-cyclopropyl-methylamine instead of 3-trifluoromethyl-5,6,7,8-tetrahydro-[1,2,4]triazolo[4,3-a]pyrazine and were isolated as a mixture of diastereoisomers.

MS (ES⁺): 293, 295 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 3.85 min.

### Example D44

### 1-{cis-[1-(3-Chlorophenyl)-4-[4-(3-phenylpropyl)piperazin-1-yl]cyclohexyl]}methanamine hydrochloride and 1-{trans-[1-(3-chlorophenyl)-4-[4-(3-phenylpropyl)piperazin-1-yl]cyclohexyl]}methanamine hydrochloride

The title compounds were prepared analogously as described in Example D1 using 1-(3-phenyl-propyl)-piperazine instead of 3-trifluoromethyl-5,6,7,8-tetrahydro-[1,2,4]triazolo[4,3-a]pyrazine and were isolated as a mixture of diastereoisomers.

MS (ES⁺): 426, 428 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 4.29, 4.45 min.

### Example D45

### 1-{cis-[1-(3-Chlorophenyl)-4-[4-(2-methoxyethyl)piperazin-1-yl]cyclohexyl]}methanamine hydrochloride and 1-{trans-[1-(3-chlorophenyl)-4-[4-(2-methoxyethyl)piperazin-1-yl]cyclohexyl]}methanamine hydrochloride

The title compounds were prepared analogously as described in Example D1 using 1-(2-methoxyethyl)-piperazine instead of 3-trifluoromethyl-5,6,7,8-tetrahydro-[1,2,4]triazolo[4,3-a]pyrazine and were isolated as a mixture of diastereoisomers.

MS (ES⁺): 366, 368 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 5.57 min.

### Example D46

### 1-[cis-{4-[4-(1,3-Benzodioxol-5-ylmethyl)piperazin-1-yl]-1-(3-chlorophenyl)cyclohexyl}]methanamine hydrochloride and 1-[trans-{4-[4-(1,3-benzodioxol-5-ylmethyl)piperazin-1-yl]-1-(3-chlorophenyl)cyclohexyl}]methanamine hydrochloride

The title compounds were prepared analogously as described in Example D1 using C-cyclopropyl-methylamine instead of 3-trifluoromethyl-5,6,7,8-tetrahydro-[1,2,4]triazolo[4,3-a]pyrazine and were isolated as a mixture of diastereoisomers.

MS (ES⁺): 442, 444 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 4.17, 4.53 min.

### Example D47

### cis-4-(Aminomethyl)-4-(3-chlorophenyl)-N-(2-thienylmethyl)cyclohexanamine hydrochloride and trans-4-(aminomethyl)-4-(3-chlorophenyl)-N-(2-thienylmethyl)cyclohexanamine hydrochloride

The title compounds were prepared analogously as described in Example D1 using C-thiophen-2-yl-methylamine instead of 3-trifluoromethyl-5,6,7,8-tetrahydro-[1,2,4]triazolo[4,3-a]pyrazine and were isolated as a mixture of diastereoisomers.

MS (ES⁺): 335, 337 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 3.40, 4.47 min.

### Example D48

### 4-{cis-[4-(Aminomethyl)-4-(3-chlorophenyl)cyclohexyl]amino}butan-1-ol hydrochloride and 4-{trans-[4-(aminomethyl)-4-(3-chlorophenyl)cyclohexyl]amino}butan-1-ol hydrochloride

The title compounds were prepared analogously as described in Example D1 using 4-aminobutan-1-ol instead of 3-trifluoromethyl-5,6,7,8-tetrahydro-[1,2,4]triazolo[4,3-a]pyrazine and were isolated as a mixture of diastereoisomers.

MS (ES⁺): 311, 313 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 3.54 min.

### Example D49

### cis-4-(Aminomethyl)-4(3-chlorophenyl)-N-[3-(1H-imidazol-1-yl)propyl]cyclohexanamine hydrochloride and trans-4-(aminomethyl)-4-(3-chlorophenyl)-N-[3-(1H-imidazol-1-yl)propyl]cyclohexanamine hydrochloride

The title compounds were prepared analogously as described in Example D1 using 3-imidazol-1-yl-propylamine instead of 3-trifluoromethyl-5,6,7,8-tetrahydro-[1,2,4]triazolo[4,3-a]pyrazine and were isolated as a mixture of diastereoisomers.

MS (ES⁺): 347, 349 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 1.09, 1.31 min.

### Example D50

### cis-4-(Aminomethyl)-4-(3-chlorophenyl)-N-(2-phenoxyethyl)cyclohexanamine hydrochloride and trans-4-(aminomethyl)-4-(3-chlorophenyl)-N-(2-phenoxyethyl)cyclohexanamine hydrochloride

The title compounds were prepared analogously as described in Example D1 using 2-phenoxy-ethylamine instead of 3-trifluoromethyl-5,6,7,8-tetrahydro-[1,2,4]triazolo[4,3-a]pyrazine and were isolated as a mixture of diastereoisomers.

MS (ES⁺): 359, 361 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 4.06, 4.71 min.

### Example D51

### 1-{cis-(3-Chlorophenyl)-4-[2-cyclopropyl-4-(trifluoromethyl)-5,8-dihydropyrido[3,4-d]pyrimidin-7(6H)-yl]cyclohexyl}methanamine hydrochloride

The title compound was prepared analogously as described in Example D1 using 2-cyclopropyl-4-trifluoromethyl-5,6,7,8-tetrahydro-pyrido[3,4-d]pyrimidine instead of 3-trifluoromethyl-5,6,7,8-tetrahydro-[1,2,4]triazolo[4,3-a]pyrazine.

MS (ES⁺): 465 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 5.75 min.

### Example D52

### 1-{trans-1-(3-Chlorophenyl)-4-[2-cyclopropyl-4-(trifluoromethyl)-5,8-dihydropyrido[3,4-d]pyrimidin-7(6H)-yl]cyclohexyl}methanamine hydrochloride

The title compound was prepared analogously as described in Examples D1 and D2 using 2-cyclopropyl-4-trifluoromethyl-5,6,7,8-tetrahydro-pyrido[3,4-d]pyrimidine instead of 3-trifluoromethyl-5,6,7,8-tetrahydro-[1,2,4]triazolo[4,3-a]pyrazine.

MS (ES⁺): 465 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 5.64 min.

### Example D53

### 2-{[cis-4-(Aminomethyl)-4-(3-chlorophenyl)cyclohexyl]amino}ethanol hydrochloride and 2-{[trans-4-(aminomethyl)-4-(3-chlorophenyl)cyclohexyl]amino}ethanol hydrochloride

The title compound was prepared according to Scheme D.

### A) A mixture of [cis-4-[2-(tert-butyl-dimethyl-silanyloxy)-ethylamino]-1-(3-chloro-phenyl)-cyclohexylmethyl]-carbamic acid tert-butyl ester and [trans-4-[2-(tert-butyl-dimethyl-silanyloxy)-ethylamino]-1-(3-chloro-phenyl)-cyclohexylmethyl]-carbamic acid tert-butyl ester

The title compounds were prepared analogously as described in Example D1 using 2-(tert-butyl-dimethyl-silanyloxy)-ethylamine instead of 3-trifluoromethyl-5,6,7,8-tetrahydro-[1,2,4]triazolo[4,3-a]pyrazine and were obtained as a mixture of diastereoisomers.

MS (ET): 497 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 2.99, 3.09 min.

### B) A mixture of [cis-1-(3-chloro-phenyl)-4-(2-hydroxy-ethylamino)-cyclohexylmethyl]-carbamic acid tert-butyl ester and [trans-1-(3-chloro-phenyl)-4-(2-hydroxy-ethylamino)-cyclohexylmethyl]-carbamic acid tert-butyl ester

A mixture of [cis-4-[2-(tert-butyl-dimethyl-silanyloxy)-ethylamino]-1-(3-chloro-phenyl)-cyclohexylmethyl]-carbamic acid tert-butyl ester and [trans-4-[2-(tert-butyl-dimethyl-silanyloxy)-ethylamino]-1-(3-chloro-phenyl)-cyclohexylmethyl]-carbamic acid tert-butyl ester (60 mg, 0.121 mmol) in tetrahydrofuran (3 mL) was treated with a 1M solution of tetrabutyl ammonium fluoride in tetrahydrofuran (240 μL) and the mixture was stirred at room temperature for 2 hours. The mixture was quenched with ammonium chloride (aq) and extracted into dichloromethane (2×30 ml). The combined extracts were washed with water and brine, dried (MgSO₄) and concentrated. The residue was purified by automated flash chromatography (Silica (4 g), eluting 0%-20% methanol in dichloromethane) to give a mixture of the title compounds as a colourless oil.

MS (ET): 327 [M+H-tBu]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 2.31, 2.41 min.

### C) 2-{[cis-4-(Aminomethyl)-4-(3-chlorophenyl)cyclohexyl]amino}ethanol hydrochloride and 2-{[trans-4-(aminomethyl)-4-(3-chlorophenyl)cyclohexyl]amino}ethanol hydrochloride

A mixture of [cis-1-(3-chloro-phenyl)-4-(2-hydroxy-ethylamino)-cyclohexylmethyl]-carbamic acid tert-butyl ester and [trans-1-(3-chloro-phenyl)-4-(2-hydroxy-ethylamino)-cyclohexylmethyl]-carbamic acid tert-butyl ester (49 mg, 0.128 mmol) in trifluoroacetic acid (1 mL) and dichloromethane (3 mL) was stirred at room temperature for 2 hours. The reaction mixture was applied to an SCX-2 ion exchange column and eluted sequentially with dichloromethane, methanol and a 2M solution of ammonia in methanol. Final purification was achieved using preparative reversed phase HPLC (acetonitrile/water containing 0.1% trifluoroacetic acid) and after treatment with excess hydrogen chloride in methanol the title compounds were obtained as a mixture of diastereoisomers.

MS (ES⁺): 283 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 1.17 min.

### Example D54

### 1-{cis-1-(3-Chlorophenyl)-4-[4-cyclopropyl-2-(trifluoromethyl)-5,8-dihydropyrido[3,4-d]pyrimidin-7(6H)-yl]cyclohexyl}methanamine hydrochloride

The title compound was prepared analogously as described in Example D1 using 4-cyclopropyl-2-trifluoromethyl-5,6,7,8-tetrahydro-pyrido[3,4-d]pyrimidine instead of 3-trifluoromethyl-5,6,7,8-tetrahydro-[1,2,4]triazolo[4,3-a]pyrazine.

MS (ES⁺): 465 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 5.80 min.

### Example D55

### 1-{trans-1-(3-Chlorophenyl)-4-[4-cyclopropyl-2-(trifluoromethyl)-5,8-dihydropyrido[3,4-d]pyrimidin-7(6H)-yl]cyclohexyl}methanamine hydrochloride

The title compound was prepared analogously as described in Example D1 using 4-cyclopropyl-2-trifluoromethyl-5,6,7,8-tetrahydro-pyrido[3,4-d]pyrimidine instead of 3-trifluoromethyl-5,6,7,8-tetrahydro-[1,2,4]triazolo[4,3-a]pyrazine.

MS (ES⁺): 465 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 5.80 min.

### Example D56

C-[8-(2,4-Difluoro-phenyl)-1,4-dioxa-spiro[4.5]dec-8-yl]-methylamine

The title compound was prepared analogously as described in Example D1 step A to step E using 2,5-difluorophenylacetonitrile instead of 3-chlorophenylacetonitrile.

MS (ES⁺): 282 [M+H]⁺.

HPLC (Zorbax SB C18, 2 min method (0-0.8 min 10-95% ACN, 0.8-1.5 min 95% ACN, 1.5-1.6 min 95-10% ACN, 1.6-2 min 10% ACN): 1.113 min.

### Example D57

C-[1-(4-Methyl-pyridin-2-yl)-4-(3-trifluoromethyl-5,6-dihydro-8H-[1,2,4]triazolo[4,3-a]pyrazin-7-yl)-cyclohexyl]-methylamine

The title compound was prepared analogously as described in Example D1 using (4-Methyl-pyridin-2-yl)-acetonitrile instead of 3-chlorophenylacetonitrile.

MS (ES⁺): 395 [M+H]⁺.

HPLC (Nucleosil, 6 min method (0-3 min 5-95% ACN, 3.5-5.5 min 95% ACN, 5.5-5.55 min 95-5% ACN, 5.55-6 min 5% ACN): 3.39 min.

### Example D58

### C-(1-Phenyl-4-piperidin-1-yl-cyclohexyl)-methylamine

The title compounds were prepared analogously as described in Example D3 using piperidine instead of 4-benzylpiperidine and were isolated as a mixture of diastereoisomers.

MS (ES⁺): 273 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 1.27-3.24 min.

### Example D59

### C-(1-Phenyl-4-pyrrolidin-1-yl-cyclohexyl)-methylamine

The title compounds were prepared analogously as described in Example D3 using pyrrolidine instead of 4-benzylpiperidine and were isolated as a mixture of diastereoisomers.

MS (ES⁺): 259 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 1.15 min.

### Example D60

### C-[1-(3-Chloro-phenyl)-4-piperazin-1-yl-cyclohexyl]-methylamine

To a solution of 4-[4-Aminomethyl-4-(3-chloro-phenyl)-cyclohexyl]-piperazine-1-carboxylic acid benzyl ester (Example 32, 37 mg, 0.083 mmol) in acetic acid (1 mL) is added a 33% hydrogen bromide solution in acetic acid (0.1 mL) before stirring at rt for 1.5 hours. The solution is passed through an SCX-2 column and eluted with DCM, methanol and 2M ammonia in methanol before evaporation and purification by preparative reversed phase HPLC (acetonitrile/water containing 0.1% trifluoroacetic acid) to give a mixture of the two isomers.

MS (ES⁺): 308 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 1.17 min.

### Example D61

### [4-Aminomethyl-4-(3-chloro-phenyl)-cyclohexyl]-phenethyl-amine

The title compounds were prepared analogously as described in Example D3 using phenylethylamine instead of 4-benzylpiperidine and were isolated as a mixture of diastereoisomers.

MS (ES⁺): 343-345 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 4.22-4.88 min.

### Example D62

### 1-{trans-1-(3-Methylphenyl)-4-[3-(trifluoromethyl)-5,6-dihydro[1,2,4]triazolo[4,3-a]pyrazin-7(8H)-yl]cyclohexyl}methanamine dihydrochloride

The title compound was prepared analogously as described in Example D1 and D2 using 3-Methyl-phenylacetonitrile instead of 3-Chlorophenylacetonitrile.

MS (ES⁺): 394 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.46 min.

### Example D63

### 1-{cis-1-(3-Methylphenyl)-4-[3-(trifluoromethyl)-5,6-dihydro[1,2,4]triazolo[4,3-a]pyrazin-7(8H)-yl]cyclohexyl}methanamine dihydrochloride

The title compound was prepared analogously as described in Example D1 using 3-Methyl-phenylacetonitrile instead of 3-Chlorophenylacetonitrile.

MS (ES⁺): 394 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.60 min.

### Example D64

### 1-[cis-4-Aminomethyl-4-(3-chloro-phenyl)-cyclohexyl]-piperidine-3-carboxylic acid ethyl ester dihydrochloride

The title compound was prepared analogously as described in Example D1 using Ethyl nipecotate instead of 3-trifluoromethyl-5,6,7,8-tetrahydro-[1,2,4]triazolo[4,3-a]pyrazine.

MS (ES⁺): 379 [M+H]⁺.

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 4.94 min.

### Example D65

### 2-{[cis-4-(Aminomethyl)-4-(3-chlorophenyl)cyclohexyl]amino}ethanol dihydrochloride

The title compound was prepared analogously as described in Example D1 using 1-Amino-2-ethanol instead of 3-trifluoromethyl-5,6,7,8-tetrahydro-[1,2,4]triazolo[4,3-a]pyrazine.

MS (ES⁺): 283 [M+H]⁺.

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 4.57 min.

### Example D66

### 4-[cis-4-Aminomethyl-4-(3-chloro-phenyl)-cyclohexylamino]-butyric acid methyl ester dihydrochloride

The title compound was prepared analogously as described in Example D1 using Methyl-4-amino butyrate hydrochloride instead of 3-trifluoromethyl-5,6,7,8-tetrahydro-[1,2,4]triazolo[4,3-a]pyrazine.

MS (ES⁺): 339 [M+H]⁺.

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 4.80 min.

### Example D67

### {3-[cis-4-Aminomethyl-4-(3-chloro-phenyl)-cyclohexylamino]-propyl}-carbamic acid benzyl ester hydrochloride

The title compound was prepared analogously as described in Example D1 using N—CBZ-1,3-diamino propane instead of 3-trifluoromethyl-5,6,7,8-tetrahydro-[1,2,4]triazolo[4,3-a]pyrazine.

MS (ES⁺): 430 [M+H]⁺.

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 5.29 min.

### Example D68

### {2-[cis-4-Aminomethyl-4-(3-chloro-phenyl)-cyclohexylamino]-ethyl}-carbamic acid benzyl ester hydrochloride

The title compound was prepared analogously as described in Example D1 using N—CBZ-1,3-diamino ethane instead of 3-trifluoromethyl-5,6,7,8-tetrahydro-[1,2,4]triazolo[4,3-a]pyrazine.

MS (ES⁺): 416 [M+H]⁺.

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 5.25 min.

### Example D69

### 1-{trans-1-(3-Chloro-phenyl)-4-[2-trifluoromethyl-5,6-dihydro-8H-[1,2,4]triazolo[1,5-a]pyrazin-7-yl]-cyclohexyl}-methylamine dihydrochloride

The title compound was prepared analogously as described in Example D1 and D2 using 2-trifluoromethyl-5,6,7,8-tetrahydro-[1,2,4]triazolo[1,5-a]pyrazine instead of 3-trifluoromethyl-5,6,7,8-tetrahydro-[1,2,4]triazolo[4,3-a]pyrazine.

MS (ES⁺): 414 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.75 min.

### Example D70

### 1-{cis-1-(3-Chloro-phenyl)-4-[2-trifluoromethyl-5,6-dihydro-8H-[1,2,4]triazolo[1,5-a]pyrazin-7-yl]-cyclohexyl}-methylamine dihydrochloride

The title compound was prepared analogously as described in Example D1 using 2-trifluoromethyl-5,6,7,8-tetrahydro-[1,2,4]triazolo[1,5-a]pyrazine instead of 3-trifluoromethyl-5,6,7,8-tetrahydro-[1,2,4]triazolo[4,3-a]pyrazine.

MS (ES⁺): 414 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.85 min.

### Example D71

### 1-[cis-1-(3-Chloro-phenyl)-4-(7-methyl-3-trifluoromethyl-7,8-dihydro-[1,2,4]triazolo[4,3c]pyrimidin-6-yl)-cyclohexyl]-methylamine dihydrochloride

The title compound was prepared analogously as described in Example D1 using 7-Methyl-3-trifluoromethyl-5,6,7,8-tetrahydro-[1,2,4]triazolo[4,3-c]pyrimidine instead of 3-trifluoromethyl-5,6,7,8-tetrahydro-[1,2,4]triazolo[4,3-a]pyrazine.

MS (ES⁺): 428 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.88 min.

### Example D72

### 1-[trans-1-(3-Chloro-phenyl)-4-(7-methyl-3-trifluoromethyl-7,8-dihydro-[1,2,4]triazolo[4,3-c]pyrimidin-6-yl)-cyclohexyl]-methylamine dihydrochloride

The title compound was prepared analogously as described in Example D1 and D2 using 7-Methyl-3-trifluoromethyl-5,6,7,8-tetrahydro-[1,2,4]triazolo[4,3-c]pyrimidine instead of 3-trifluoromethyl-5,6,7,8-tetrahydro-[1,2,4]triazolo[4,3-a]pyrazine.

MS (ES⁺): 428 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.80 min.

### Example D73

### 1-[cis-1-(3-Chloro-phenyl)-4-(7-ethyl-3-trifluoromethyl-7,8-dihydro-[1,2,4]triazolo[4,3-c]pyrimidin-6-yl)-cyclohexyl]-methylamine dihydrochloride

The title compound was prepared analogously as described in Example D1 using 7-Ethyl-3-trifluoromethyl-5,6,7,8-tetrahydro-[1,2,4]triazolo[4,3-c]pyrimidine instead of 3-trifluoromethyl-5,6,7,8-tetrahydro-[1,2,4]triazolo[4,3-a]pyrazine.

MS (ES⁺): 442 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.10 min.

### Example D74

### 1-[trans-1-(3-Chloro-phenyl)-4-(7-ethyl-3-trifluoromethyl-7,8-dihydro-[1,2,4]triazolo[4,3-c]pyrimidin-6-yl)-cyclohexyl]-methylamine dihydrochloride

The title compound was prepared analogously as described in Example D1 and D2 using 7-Ethyl-3-trifluoromethyl-5,6,7,8-tetrahydro-[1,2,4]triazolo[4,3-c]pyrimidine instead of 3-trifluoromethyl-5,6,7,8-tetrahydro-[1,2,4]triazolo[4,3-a]pyrazine.

MS (ES⁺): 442 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.01 min.

### Example D75

### 1-[cis-1-(3-Chloro-phenyl)-4-(4-cyclopropyl-2-methoxy-5,8-dihydro-6H-pyrido[3,4-d]pyrimidin-7-yl)-cyclohexyl]-methylamine dihydrochloride

The title compound was prepared analogously as described in Example D1 using 4-Cyclopropyl-2-methoxy-5,6,7,8-tetrahydro-pyrido[3,4-d]pyrimidine instead of 3-trifluoromethyl-5,6,7,8-tetrahydro-[1,2,4]triazolo[4,3-a]pyrazine.

MS (ES⁺): 427 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.70 min.

### Example D76

### {-7-[cis-4-Aminomethyl-4-(3-chloro-phenyl)-cyclohexyl]-4-cyclopropyl-5,6,7,8-tetrahydro-pyrido[3,4-d]pyrimidin-2-yl}-dimethylamine dihydrochloride

The title compound was prepared analogously as described in Example D1 using (4-Cyclopropyl-5,6,7,8-tetrahydro-pyrido[3,4-d]pyrimidin-2-yl)-dimethyl-amine instead of 3-trifluoromethyl-5,6,7,8-tetrahydro-[1,2,4]triazolo[4,3-a]pyrazine.

MS (ES⁺): 440 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.93 min.

### Example D77

### [cis-4-Aminomethyl-4-(3-chloro-phenyl)-cyclohexyl]-[2-(3-methanesulfonyl-phenyl)-ethyl]-amine dihydrochloride

The title compound was prepared analogously as described in Example D1 using 2-(3-Methanesulfonyl-phenyl)-ethylamine instead of 3-trifluoromethyl-5,6,7,8-tetrahydro-[1,2,4]triazolo[4,3-a]pyrazine.

MS (ES⁺): 421 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.50 min.

### Example D78

### [cis-4-Aminomethyl-4-(3-chloro-phenyl)-cyclohexyl]-(4-methanesulfonyl-benzyl)-amine dihydrochloride

The title compound was prepared analogously as described in Example D1 using 4-Methanesulfonyl-benzylamine instead of 3-trifluoromethyl-5,6,7,8-tetrahydro-[1,2,4]triazolo[4,3-a]pyrazine.

MS (ES⁺): 407 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.33 min.

### Example D79

### 6-[cis-4-Aminomethyl-4-(3-chloro-phenyl)-cyclohexyl]-4-methyl-5,6,7,8-tetrahydro-pyrido[4,3-d]pyrimidin-2-ylamine dihydrochloride

The title compound was prepared analogously as described in Example D1 using 4-Methyl-5,6,7,8-tetrahydro-pyrido[4,3-d]pyrimidin-2-ylamine instead of 3-trifluoromethyl-5,6,7,8-tetrahydro-[1,2,4]triazolo[4,3-a]pyrazine.

MS (ES⁺): 386, 388 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 2.90 min.

### Example D80

### 1-[cis-1-(3-Ethynyl-phenyl)-4-(3-trifluoromethyl-5,6-dihydro-8H-[1,2,4]-triazolo[4,3-a]pyrazin-7-yl)-cyclohexyl]-methylamine dihydrochloride

The title compound was prepared analogously as described in Example D1 using 3-Ethynyl-phenylacetonitrile instead of 3-Chlorophenylacetonitrile.

MS (ES⁺): 404 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.44 min.

### Example D81

### 1-[cis-1-(4-Methyl-pyridin-2-yl)-4-(3-trifluoromethyl-5,6-dihydro-8H-[1,2,4]triazolo[4,3-a]pyrazin-7-yl)-cyclohexyl]-methylamine dihydrochloride

The title compound was prepared analogously as described in Example D1 using (4-Methyl-pyridin-2-yl)-acetonitrile instead of 3-Chlorophenylacetonitrile.

MS (ES⁺): 395 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 0.89 min.

### Example D82

### {6-[cis-4-Aminomethyl-4-(3-chloro-phenyl)-cyclohexyl]-4-methyl-5,6,7,8-tetrahydro-pyrido[4,3-d]pyrimidin-2-yl}-cyclopropylmethyl-amine dihydrochloride

The title compound was prepared analogously as described in Example D1 using Cyclopropylmethyl-(4-methyl-5,6,7,8-tetrahydro-pyrido[4,3-d]pyrimidin-2-yl)-amine instead of 3-trifluoromethyl-5,6,7,8-tetrahydro-[1,2,4]triazolo[4,3-a]pyrazine.

MS (ES⁺): 440 [M−H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.36 min.

### Example D83

### 1-{trans-1-(3-Methylphenyl)-4-[3-(trifluoromethyl)-5,6-dihydro[1,2,4]triazolo[4,3-a]pyrazin-7(8H)-yl]cyclohexyl}methanamine dihydrochloride

The title compound was prepared analogously as described in Example D1 and D2 using 3-Methyl-phenylacetonitrile instead of 3-Chlorophenylacetonitrile and using 2-trifluoromethyl-5,6,7,8-tetrahydro-[1,2,4]triazolo[1,5-a]pyrazine instead of 3-trifluoromethyl-5,6,7,8-tetrahydro-[1,2,4]triazolo[4,3-a]pyrazine.

MS (ES⁺): 394 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.72 min.

### Example D84

### 1-{cis-1-(3-Methylphenyl)-4-[3-(trifluoromethyl)-5,6-dihydro[1,2,4]triazolo[4,3-a]pyrazin-7(8H)-yl]cyclohexyl}methanamine dihydrochloride

The title compound was prepared analogously as described in Example D1 using 3-Methyl-phenylacetonitrile instead of 3-Chlorophenylacetonitrile and using 2-trifluoromethyl-5,6,7,8-tetrahydro-[1,2,4]triazolo[1,5-a]pyrazine instead of 3-trifluoromethyl-5,6,7,8-tetrahydro-[1,2,4]triazolo[4,3-a]pyrazine.

MS (ES⁺): 394 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.82 min.

### Example D85

### 2-[trans-4-Aminomethyl-4-(3-chloro-phenyl)-cyclohexyl]-2,3-dihydro-isoindol-1-one dihydrochloride

The title compound was prepared analogously as described in Example D1 and D2 using 2-carbomethoxybenzylamine hydrochloride instead of 3-trifluoromethyl-5,6,7,8-tetrahydro-[1,2,4]triazolo[4,3-a]pyrazine.

MS (ES⁺): 355 [M+H]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 2.57 min.

### Example D86

### 2-[cis-4-Aminomethyl-4-(3-chloro-phenyl)-cyclohexyl]-2,3-dihydro-isoindol-1-one dihydrochloride

The title compound was prepared analogously as described in Example D1 using 2-carbomethoxybenzylamine hydrochloride instead of 3-trifluoromethyl-5,6,7,8-tetrahydro-[1,2,4]triazolo[4,3-a]pyrazine.

MS (ES⁺): 355 [M+H]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 2.44 min.

### Example D87

### 1-[cis-1-(5-Chloro-2-fluoro-phenyl)-4-(2-trifluoromethyl-5,6-dihydro-8H-[1,2,4]triazolo[1,5-a]pyrazin-7-yl)-cyclohexyl]-methylamine dihydrochloride

The title compound was prepared analogously as described in Example D1 using 5-Chloro-2-fluorophenylacetonitrile instead of 3-Chlorophenylacetonitrile.

MS (ES⁺): 432 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.79 min.

### Example D88

### 1-[cis-1-(3-Chloro-phenyl)-4-(5,6-dihydro-8H-[1,2,4]triazolo[1,5-a]pyrazin-7-yl)-cyclohexyl]-methylamine dihydrochloride

The title compound was prepared analogously as described in Example D1 using 5,6,7,8-Tetrahydro-[1,2,4]triazolo[1,5-a]pyrazine instead of 3-trifluoromethyl-5,6,7,8-tetrahydro-[1,2,4]triazolo[4,3-a]pyrazine.

MS (ES⁺): 346 [M+H]⁺.

HPLC (Zorbax SB C18, 2 min method (0-0.8 min 10-95% ACN, 0.8-1.5 min 95% ACN, 1.5-1.6 min 95-10% ACN, 1.6-2 min 10% ACN): 0.3 min.

### Example D89

### 1-[trans-1-(3-Chloro-phenyl)-4-(5,6-dihydro-8H-[1,2,4]triazolo[1,5-a]pyrazin-7-yl)-cyclohexyl]-methylamine dihydrochloride

The title compound was prepared analogously as described in Example D2 using 5,6,7,8-Tetrahydro-[1,2,4]triazolo[1,5-a]pyrazine instead of 3-trifluoromethyl-5,6,7,8-tetrahydro-[1,2,4]triazolo[4,3-a]pyrazine.

MS (ES⁺): 346 [M+H]⁺.

HPLC (Zorbax SB C18, 2 min method (0-0.8 min 10-95% ACN, 0.8-1.5 min 95% ACN, 1.5-1.6 min 95-10% ACN, 1.6-2 min 10% ACN): 0.25 min.

### Example D90

### 1-[trans-1-(3-Chloro-phenyl)-4(5,6-dihydro-8H-[1,2,4]triazolo[4,3-a]pyrazin-7-yl)-cyclohexyl]-methylamine dihydrochloride

The title compound was prepared analogously as described in Example D2 using 5,6,7,8-Tetrahydro-[1,2,4]triazolo[4,3-a]pyrazine instead of 3-trifluoromethyl-5,6,7,8-tetrahydro-[1,2,4]triazolo[4,3-a]pyrazine.

MS (ES⁺): 346, 348 [M+H]⁺.

HPLC (Nucleosil, 6 min method (0-3 min 5-95% ACN, 3.5-5.5 min 95% ACN, 5.5-5.55 min 95-5% ACN, 5.55-6 min 5% ACN): 3.09 min.

### Example D91

### 1-[cis-1-(3-Chloro-phenyl)-4-(5,6-dihydro-8H-[1,2,4]triazolo[4,3-a]pyrazin-7-yl)-cyclohexyl]-methylamine dihydrochloride

The title compound was prepared analogously as described in Example D1 using 5,6,7,8-Tetrahydro-[1,2,4]triazolo[4,3-a]pyrazine instead of 3-trifluoromethyl-5,6,7,8-tetrahydro-[1,2,4]triazolo[4,3-a]pyrazine.

MS (ES⁺): 346, 348 [M+H]⁺.

HPLC (Nucleosil, 6 min method (0-3 min 5-95% ACN, 3.5-5.5 min 95% ACN, 5.5-5.55 min 95-5% ACN, 5.55-6 min 5% ACN): 3.57 min.

### Example D92

### 3-{7-[4-Aminomethyl-4-(3-chloro-phenyl)-cyclohexyl]-5,6,7,8-tetrahydro-pyrido[3,4-d]pyrimidin-4-yl}-benzoic acid ethyl ester

The title compound was prepared analogously as described in Example D1 using 3-(5,6,7,8-Tetrahydro-pyrido[3,4-d]pyrimidin-4-yl)-benzoic acid ethyl ester instead of 3-trifluoromethyl-5,6,7,8-tetrahydro-[1,2,4]triazolo[4,3-a]pyrazine.

MS (ES⁺): 505 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 4.06 min.

### Example D93

### 1-[trans-1-(3-Chloro-phenyl)-4-(2-methyl-6,7-dihydro-4H-oxazolo[5,4-c]pyridin-5-yl)-cyclohexyl]-methylamine dihydrochloride

The title compound was prepared analogously as described in Example D2 using 2-Methyl-4,5,6,7-tetrahydro-oxazolo[5,4-c]pyridine instead of 3-trifluoromethyl-5,6,7,8-tetrahydro-[1,2,4]triazolo[4,3-a]pyrazine.

MS (ES⁺): 361 [M+H]⁺.

HPLC (Nucleosil, 6 min method (0-3 min 5-95% ACN, 3.5-5.5 min 95% ACN, 5.5-5.55 min 95-5% ACN, 5.55-6 min 5% ACN): 3.51 min.

### Example D94

### 1-[cis-1-(3-Chloro-phenyl)-4-(2-methyl-6,7-dihydro-4H-oxazolo[5,4-c]pyridin-5-yl)-cyclohexyl]-methylamine dihydrochloride

The title compound was prepared analogously as described in Example D1 using 2-Methyl-4,5,6,7-tetrahydro-oxazolo[5,4-c]pyridine instead of 3-trifluoromethyl-5,6,7,8-tetrahydro-[1,2,4]triazolo[4,3-a]pyrazine.

MS (ES⁺): 362 [M+H]⁺.

HPLC (Nucleosil, 6 min method (0-3 min 5-95% ACN, 3.5-5.5 min 95% ACN, 5.5-5.55 min 95-5% ACN, 5.55-6 min 5% ACN): 3.67 min.

### Example D95

### 1-[cis-4-Aminomethyl-4-(3-chloro-phenyl)-cyclohexyl]-4-phenyl-piperazine-2,3-dione dihydrochloride

The title compound was prepared analogously as described in Example D1 using N-(2-Amino-ethyl)-N-phenyl-oxalamic acid ethyl ester instead of 3-trifluoromethyl-5,6,7,8-tetrahydro-[1,2,4]triazolo[4,3-a]pyrazine. The open ring after reductive amination step closed itself during workup.

MS (ES⁺): 412 [M+H]⁺.

HPLC (Agilent Eclipse XDB-C18, 1.8 μm 4.6×50 mm, 8 min method (0-6 min 20-100% ACN, 6.0-7.5 min 100% ACN, 7.5-8.0 min 100-20% ACN): 2.62 min.

### Example D96

### 1-[cis-1-(2,5-Dichloro-phenyl)-4-(3-trifluoromethyl-5,6-dihydro-8H-[1,2,4]triazolo[4,3-a]pyrazin-7-yl)-cyclohexyl]-methylamine dihydrochloride

The title compound was prepared analogously as described in Example D1 using 2,5-Dichlorophenylacetonitrile instead of 3-Chlorophenylacetonitrile.

MS (ES⁺): 448 [M+H]⁺.

### Example D97

### N-(cis-3-{2-[4-Aminomethyl-4-(3-chloro-phenyl)-cyclohexylamino]-ethyl}-phenyl)-methanesulfonamide dihydrochloride

The title compound was prepared analogously as described in Example D1 using N-[3-(2-Amino-ethyl)-phenyl]-methanesulfonamide instead of 3-trifluoromethyl-5,6,7,8-tetrahydro-[1,2,4]triazolo[4,3-a]pyrazine.

### Example D98

### 1-[cis-4-(3-Trifluoromethyl-5,6-dihydro-8H-[1,2,4]triazolo[4,3-a]pyrazin-7-yl)-1-(3-trifluoromethyl-phenyl)-cyclohexyl]-methylamine ditrifluoroacetate

The title compound was prepared analogously as described in Example D1 using 3-(Trifluoromethyl)-phenylacetonitrile instead of 3-Chlorophenylacetonitrile.

MS (ES⁺): 448 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.90 min.

### Example D99

### 1-[trans-4-(3-Trifluoromethyl-5,6-dihydro-8H-[1,2,4]triazolo[4,3-a]pyrazin-7-yl)-1-(3-trifluoromethyl-phenyl)-cyclohexyl]-methylamine

The title compound was prepared analogously as described in Example D2 using 3-(Trifluoromethyl)-phenylacetonitrile instead of 3-Chlorophenylacetonitrile.

MS (ES⁺): 448 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.81 min.

### Example D100

### (S)-2-[cis-4-Aminomethyl-4-(3-chloro-phenyl)-cyclohexyl]-hexahydro-pyrido[1,2-a]pyrazine-1,4-dione dihydrochloride

The title compound was prepared analogously as described in Example D1 using (S)-1-(2-Amino-acetyl)-piperidine-2-carboxylic acid methyl ester instead of 3-trifluoromethyl-5,6,7,8-tetrahydro-[1,2,4]triazolo[4,3-a]pyrazine. The open ring closed itself during reductive amination step.

MS (ES⁺): 390 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.21 min.

### Example D101

### 2-[trans-4-Aminomethyl-4-(3-chloro-phenyl)-cyclohexyl]-1,4-dihydro-2H-isoquinolin-3-one hydrochloride

The title compound was prepared analogously as described in Example D2 using Methyl-2-aminoethylphenylacetate hydrochloride instead of 3-trifluoromethyl-5,6,7,8-tetrahydro-[1,2,4]triazolo[4,3-a]pyrazine. The open ring closed itself during reductive amination step.

MS (ES⁺): 369 [M+H]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 2.53 min.

### Example D102

### 2-[cis-4-Aminomethyl-4-(3-chloro-phenyl)-cyclohexyl]-1,4-dihydro-2H-isoquinolin-3-one hydrochloride

The title compound was prepared analogously as described in Example D1 using Methyl-2-aminoethylphenylacetate hydrochloride instead of 3-trifluoromethyl-5,6,7,8-tetrahydro-[1,2,4]triazolo[4,3-a]pyrazine. The open ring closed itself during reductive amination step.

MS (ES⁺): 369 [M+H]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 2.44 min.

### Example D103

### 3-{7-[cis-4-Aminomethyl-4-(3-chloro-phenyl)-cyclohexyl]-5,6,7,8-tetrahydro-pyrido[3,4-d]pyrimidin-4-yl}-benzoic acid trifluoroacetate

The title compound was prepared analogously as described in Example D1 step A to H using 3-(5,6,7,8-Tetrahydro-pyrido[3,4-d]pyrimidin-4-yl)-benzoic acid ethyl ester instead of 3-trifluoromethyl-5,6,7,8-tetrahydro-[1,2,4]triazolo[4,3-a]pyrazine to afford 3-{7-[4-(tert-Butoxycarbonylamino-methyl)-4-(cis-3-chloro-phenyl)-cyclohexyl]-5,6,7,8-tetrahydro-pyrido[3,4-d]pyrimidin-4-yl}-benzoic acid ethyl ester and 3-{7-[4-(tert-Butoxycarbonylamino-methyl)-4-(trans-3-chloro-phenyl)-cyclohexyl]-5,6,7,8-tetrahydro-pyrido[3,4-d]pyrimidin-4-yl}benzoic acid ethyl ester followed by step

### I) 3-{7-[4-(tert-Butoxycarbonylamino-methyl)-4-(cis-3-chloro-phenyl)-cyclohexyl]-5,6,7,8-tetrahydro-pyrido[3,4-d]pyrimidin-4-yl}-benzoic acid

To a solution of 3-{7-[4-(tert-Butoxycarbonylamino-methyl)-4-(cis-3-chloro-phenyl)-cyclohexyl]-5,6,7,8-tetrahydro-pyrido[3,4-d]pyrimidin-4-yl}-benzoic acid ethyl ester (45 mg, 0.067 mmol) in tetrahydrofurane (0.7 ml) and water (0.3 ml) was added Lithium hydroxide (9 mg, 0.213 mmol). The mixture was stirred at room temperature for 16 h. The reaction mixture was directly purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were lyophilized in vacuo to give the title compound as a pale yellow powder.

MS (ES⁺): 577 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 4.77 min.

### J) 3-{7-[cis-4-Aminomethyl-4-(3-chloro-phenyl)-cyclohexyl]-5,6,7,8-tetrahydro-pyrido[3,4-d]pyrimidin-4-yl}-benzoic acid trifluoroacetate

To 3-{7-[4-(tert-Butoxycarbonylamino-methyl)-4-(cis-3-chloro-phenyl)-cyclohexyl]-5,6,7,8-tetrahydro-pyrido[3,4-d]pyrimidin-4-yl}-benzoic acid (32 mg, 0.045 mmol) in dioxane (0.3 ml) was added 4N hydrogen chloride solution in dioxane (223 μl). The reaction mixture stirred at room temperature for 2 h, then the dioxane solution was removed with a pipette. The residue was treated with diethyl ether in ultrasonic bath. The etheric phase was removed with a pipette. The residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were lyophilized in vacuo to give the title compound.

MS (ES⁺): 477 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.38 min.

### Example D104

### 1-[cis-4-Aminomethyl-4-(3-chloro-phenyl)-cyclohexyl]-4-cyclobutyl-piperazine-2,5-dione

The title compound was prepared analogously as described in Example D1 using [(2-Amino-acetyl)cyclobutyl-amino]-acetic acid ethyl ester hydrochloride instead of 3-trifluoromethyl-5,6,7,8-tetrahydro-[1,2,4]triazolo[4,3-a]pyrazine. The open ring closed itself during reductive amination step.

MS (ES⁺): 390 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.85 min.

### Example D105

### 4-{4-[4-Aminomethyl-4-(3-chloro-phenyl)-cyclohexyl]-2,5-dioxo-piperazin-1-yl}-piperidine-1-carboxylic acid ethyl ester

The title compound was prepared analogously as described in Example D1 using 4-[(2-Amino-acetyl)-ethoxycarbonylmethyl-amino]-piperidine-1-carboxylic acid ethyl ester hydrochloride instead of 3-trifluoromethyl-5,6,7,8-tetrahydro-[1,2,4]triazolo[4,3-a]pyrazine. The open ring closed itself during reductive amination step.

MS (ES⁺): 491 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.91 min.

### Example D106

### 1-[cis-4-Aminomethyl-4-(3-chloro-phenyl)-cyclohexyl]-4-benzyl-piperazine-2,5-dione

The title compound was prepared analogously as described in Example D1 using [(2-Amino-acetyl)-benzyl-amino]-acetic acid ethyl ester instead of 3-trifluoromethyl-5,6,7,8-tetrahydro-[1,2,4]triazolo[4,3-a]pyrazine. The open ring closed itself during reductive amination step.

MS (ES⁺): 426 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.17 min.

### Example D107

### 1-[cis-4-Aminomethyl-4-(3-chloro-phenyl)-cyclohexyl]-4-(2-methoxy-ethyl)-piperazine-2,5-dione

The title compound was prepared analogously as described in Example D1 using [(2-Amino-acetyl)-(2-methoxy-ethyl)-amino]-acetic acid ethyl ester instead of 3-trifluoromethyl-5,6,7,8-tetrahydro-[1,2,4]triazolo[4,3-a]pyrazine. The open ring closed itself during reductive amination step.

MS (ES⁺): 394 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.84 min.

### Example DA1

### 7-[cis-4-Aminomethyl-4-(3-chloro-phenyl)-cyclohexyl]-3-trifluoromethyl-6,7-dihydro-5H-[1,2,4]triazolo[4,3-a]pyrazin-8-one dihydrochloride

The title compound was prepared analogously as described in Example D1 step A to H followed by step

### I) [1-(cis-3-Chloro-phenyl)-4-(8-oxo-3-trifluoromethyl-5,6-dihydro-8H-[1,2,4]triazolo[4,3-a]pyrazin-7-yl)-cyclohexylmethyl]-carbamic acid tert-butyl ester

To a solution of [1-(cis-3-Chloro-phenyl)-4-(3-trifluoromethyl-5,6-dihydro-8H-[1,2,4]triazolo[4,3-a]pyrazin-7-yl)-cyclohexylmethyl]-carbamic acid tert-butyl ester (60 mg, 0.117 mmol) in acetonitrile (1 ml) and chloroform (1 ml) was added a solution of sodium periodate (103 mg, 0.48 mmol) in water (1.5 ml) and rutheniumdioxide hydrate (1 mg, 0.008 mmol). The mixture was stirred vigorously for 40 mins at room temperature, then cautiously quenched with diethylether (10 ml) and diluted with water (10 ml). The product was extracted into ethyl acetate. The combined organic extracts were dried over sodium sulfate and filtered over Celite. The filtrate was concentrated in vacuo to give the title ompound as a pale yellow solid.

MS (ES⁺): 528 [M+H]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 3.65 min.

### J) 7-[cis-4-Aminomethyl-4-(3-chloro-phenyl)-cyclohexyl]-3-trifluoromethyl-6,7-dihydro-5H-[1,2,4]triazolo[4,3-a]pyrazin-8-one dihydrochloride

Trifluoroacetic acid (1 mL) was added to a solution of [1-(cis-3-Chloro-phenyl)-4-(8-oxo-3-trifluoromethyl-5,6-dihydro-8H-[1,2,4]triazolo[4,3-a]pyrazin-7-yl)-cyclohexylmethyl]-carbamic acid tert-butyl ester (55 mg, 0.104 mmol) in dichloromethane (1 mL) and the reaction stirred at room temperature for 1 h. The reaction mixture was concentrated in vacuo and the residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were lyophilized in vacuo to give the formate salt of the title compound, which was dissolved in methanol and treated with an excess of 2M hydrogen chloride in methanol. Removal of the volatiles gave the title compound as a white solid.

MS (ES⁺): 428 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.81 min.

### Example DA2

### 7-[trans-4-Aminomethyl-4-(3-chloro-phenyl)-cyclohexyl]-3-trifluoromethyl-6,7-dihydro-5H-[1,2,4]triazolo[4,3-a]pyrazin-8-one dihydrochloride

The title compound was prepared analogously as described in Example DA1, step I from [1-(trans-3-Chloro-phenyl)-4-(8-oxo-3-trifluoromethyl-5,6-dihydro-8H-[1,2,4]triazolo[4,3-a]pyrazin-7-yl)-cyclohexylmethyl]-carbamic acid tert-butyl ester.

MS (ES⁺): 428 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.84 min.

### Example DA3

### 7-[trans-4-Aminomethyl-4-(3-chloro-phenyl)-cyclohexyl]-2-trifluoromethyl-6,7-dihydro-5H-[1,2,4]triazolo[1,5-a]pyrazin-8-one dihydrochloride

The title compound was prepared analogously as described in Example DA1 and DA2 using 2-trifluoromethyl-5,6,7,8-tetrahydro-[1,2,4]triazolo[1,5-a]pyrazine instead of 3-trifluoromethyl-5,6,7,8-tetrahydro-[1,2,4]triazolo[4,3-a]pyrazine (step H).

MS (ES⁺): 428 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.95 min.

### Example DA4

### 7-[cis-4-Aminomethyl-4-(3-chloro-phenyl)-cyclohexyl]-2-trifluoromethyl-6,7-dihydro-5H-[1,2,4]triazolo[1,5-a]pyrazin-8-one dihydrochloride

The title compound was prepared analogously as described in Example DA1 using 2-trifluoromethyl-5,6,7,8-tetrahydro-[1,2,4]triazolo[1,5-a]pyrazine instead of 3-trifluoromethyl-5,6,7,8-tetrahydro-[1,2,4]triazolo[4,3-a]pyrazine (step H).

MS (ES⁺): 428 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.96 min.

### Example DA5

### 7-[cis-4-Aminomethyl-4-(3-chloro-phenyl)-cyclohexyl]-4-cyclopropyl-2-trifluoromethyl-6,7-dihydro-5H-pyrido[3,4-d]pyrimidin-8-one dihydrochloride

The title compound was prepared analogously as described in Example DA1 using 4-cyclopropyl-2-trifluoromethyl-5,6,7,8-tetrahydro-pyrido[3,4-d]pyrimidine instead of 3-trifluoromethyl-5,6,7,8-tetrahydro-[1,2,4]triazolo[4,3-a]pyrazine.

MS (ES⁺): 479 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 4.63 min.

### Example DA6

### 7-(trans-4-Aminomethyl-4-m-tolyl-cyclohexyl)-2-trifluoromethyl-6,7-dihydro-5H-[1,2,4]triazolo[1,5-a]pyrazin-8-one dihydrochloride

The title compound was prepared analogously as described in Example DA1 and DA2 using 3-Methyl-phenylacetonitrile instead of 3-Chlorophenylacetonitrile and using 2-trifluoromethyl-5,6,7,8-tetrahydro-[1,2,4]triazolo[1,5-a]pyrazine instead of 3-trifluoromethyl-5,6,7,8-tetrahydro-[1,2,4]triazolo[4,3-a]pyrazine.

MS (ES⁺): 408 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.04 min.

### Example DA7

### 7-(cis-4-Aminomethyl-4-m-tolyl-cyclohexyl)-2-trifluoromethyl-6,7-dihydro-5H-[1,2,4]triazolo[1,5-a]pyrazin-8-one dihydrochloride

The title compound was prepared analogously as described in Example DA1 using 3-Methyl-phenylacetonitrile instead of 3-Chlorophenylacetonitrile and using 2-trifluoromethyl-5,6,7,8-tetrahydro-[1,2,4]triazolo[1,5-a]pyrazine instead of 3-trifluoromethyl-5,6,7,8-tetrahydro-[1,2,4]triazolo[4,3-a]pyrazine.

MS (ES⁺): 408 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.06 min.

### Example DA8

### 2-[cis-4-Aminomethyl-4-(3-chloro-phenyl)-cyclohexyl]-3,4-dihydro-2H-isoquinolin-1-one

The title compound was prepared analogously as described in Example DA1 using 1,2,3,4-Tetrahydro-isoquinoline instead of 3-trifluoromethyl-5,6,7,8-tetrahydro-[1,2,4]triazolo[4,3-a]pyrazine.

MS (ES⁺): 396, 371 [M+H]⁺.

### Example DA9

### N-{6-[cis-4-Aminomethyl-4-(3-chloro-phenyl)-cyclohexyl]-4-methyl-5-oxo-5,6,7,8-tetrahydro-pyrido[4,3-d]pyrimidin-2-yl}-acetamide

The title compound was prepared analogously as described in Example DA1 using N-(4-Methyl-5,6,7,8-tetrahydro-pyrido[4,3-d]pyrimidin-2-yl)-acetamide instead of 3-trifluoromethyl-5,6,7,8-tetrahydro-[1,2,4]triazolo[4,3-a]pyrazine.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.67 min.

### Example DA10

### 7-(cis-4-Aminomethyl-4-m-tolyl-cyclohexyl)-3-trifluoromethyl-6,7-dihydro-5H-[1,2,4]triazolo[4,3-a]pyrazin-8-one dihydrochloride

The title compound was prepared analogously as described in Example DA1 using 3-Methyl-phenylacetonitrile instead of 3-Chlorophenylacetonitrile.

MS (ES⁺): 408 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.82 min.

### Example DA11

### 2-Amino-6-[cis-4-aminomethyl-4-(3-chloro-phenyl)-cyclohexyl]-4-methyl-7,8-dihydro-6H-pyrido[4,3-d]pyrimidin-5-one

The title compound was prepared analogously as described in Example DA1 using 4-Methyl-5,6,7,8-tetrahydro-pyrido[4,3-d]pyrimidin-2-ylamine instead of 3-trifluoromethyl-5,6,7,8-tetrahydro-[1,2,4]triazolo[4,3-a]pyrazine.

MS (ES⁺): 400 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.43 min.

### Example DA12

### 7-[trans-4-Aminomethyl-4-(3-chloro-phenyl)-cyclohexyl]-6,7-dihydro-5H-[1,2,4]triazolo[1,5-a]pyrazin-8-one dihydrochloride

The title compound was prepared analogously as described in Example DA1 and DA2 using 5,6,7,8-Tetrahydro-[1,2,4]triazolo[1,5-a]pyrazine instead of 3-trifluoromethyl-5,6,7,8-tetrahydro-[1,2,4]triazolo[4,3-a]pyrazine (step H).

MS (ES⁺): 360 [M+H]⁺.

HPLC (Zorbax SB C18, 2 min method (0-0.8 min 10-95% ACN, 0.8-1.5 min 95% ACN, 1.5-1.6 min 95-10% ACN, 1.6-2 min 10% ACN): 0.25 min.

### Example DA13

### 7-(trans-4-Aminomethyl-4-m-tolyl-cyclohexyl)-3-trifluoromethyl-6,7-dihydro-5H-[1,2,4]triazolo[4,3-a]pyrazin-8-one dihydrochloride

The title compound was prepared analogously as described in Example DA1 and DA2 using 3-Methyl-phenylacetonitrile instead of 3-Chlorophenylacetonitrile.

MS (ES⁺): 408 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.92 min.

### Example DA14

### 7-[cis-4-Aminomethyl-4-(3-chloro-phenyl)-cyclohexyl]-6,7-dihydro-5H-[1,2,4]triazolo[1,5-a]pyrazin-8-one dihydrochloride

The title compound was prepared analogously as described in Example DA1 using 5,6,7,8-Tetrahydro-[1,2,4]triazolo[1,5-a]pyrazine instead of 3-trifluoromethyl-5,6,7,8-tetrahydro-[1,2,4]triazolo[4,3-a]pyrazine (step H).

MS (ES⁺): 360 [M+H]⁺.

### Example DA15

### 7-[trans-4-Aminomethyl-4-(5-chloro-2-fluoro-phenyl)-cyclohexyl]-3-trifluoromethyl-6,7-dihydro-5H-[1,2,4]triazolo[4,3-a]pyrazin-8-one dihydrochloride

The title compound was prepared analogously as described in Example DA1 and DA2 using 5-Chloro-2-fluorophenylacetonitrile instead of 3-Chlorophenylacetonitrile.

MS (ES⁺): 446 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.85 min.

### Example DA16

### 7-[cis-4-Aminomethyl-4-(5-chloro-2-fluoro-phenyl)-cyclohexyl]-3-trifluoromethyl-6,7-dihydro-5H-[1,2,4]triazolo[4,3-a]pyrazin-8-one dihydrochloride

The title compound was prepared analogously as described in Example DA1 and DA2 using 5-Chloro-2-fluorophenylacetonitrile instead of 3-Chlorophenylacetonitrile.

MS (ES⁺): 446 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.91 min.

### Example DA17

### 7-[trans-4-Aminomethyl-4-(3-chloro-phenyl)-cyclohexyl]-6,7-dihydro-5H-[1,2,4]triazolo [4,3-a]pyrazin-8-one dihydrochloride

The title compound was prepared analogously as described in Example DA1 and DA2 using 5,6,7,8-Tetrahydro-[1,2,4]triazolo[4,3-a]pyrazine instead of 3-trifluoromethyl-5,6,7,8-tetrahydro-[1,2,4]triazolo[4,3-a]pyrazine.

MS (ES⁺): 360 [M+H]⁺.

HPLC (Nucleosil, 6 min method (0-3 min 5-95% ACN, 3.5-5.5 min 95% ACN, 5.5-5.55 min 95-5% ACN, 5.55-6 min 5% ACN): 3.69 min.

### Example DA18

### 7-[cis-4-Aminomethyl-4-(3-chloro-phenyl)-cyclohexyl]-6,7-dihydro-5H-[1,2,4]triazolo[4,3-a]pyrazin-8-one dihydrochloride

The title compound was prepared analogously as described in Example DA1 using 5,6,7,8-Tetrahydro-[1,2,4]triazolo[4,3-a]pyrazine instead of 3-trifluoromethyl-5,6,7,8-tetrahydro-[1,2,4]triazolo[4,3-a]pyrazine.

MS (ES⁺): 360 [M+H]⁺.

HPLC (Nucleosil, 6 min method (0-3 min 5-95% ACN, 3.5-5.5 min 95% ACN, 5.5-5.55 min 95-5% ACN, 5.55-6 min 5% ACN): 3.85 min.

### Example DA19

### 7-[cis-4-Aminomethyl-4-(3-chloro-phenyl)-cyclohexyl]-4-cyclopropyl-6,7-dihydro-5H-pyrido[3,4-d]pyrimidin-8-one

The title compound was prepared analogously as described in Example DA1 using 4-Cyclopropyl-5,6,7,8-tetrahydro-pyrido[3,4-d]pyrimidine instead of 3-trifluoromethyl-5,6,7,8-tetrahydro-[1,2,4]triazolo[4,3-a]pyrazine.

MS (ES⁺): 411 [M+H]⁺.

HPLC (Nucleosil C-18HD 4×70 mm 3 μm, 8 min method (0-6 min 20-100% ACN, 6.0-7.5 min 100% ACN, 7.5-8.0 min 100-20% ACN): 2.83 min.

### Example DA20

### 7-[cis-4-Aminomethyl-4-(3-chloro-phenyl)-cyclohexyl]-4-(3-methanesulfonyl-phenyl)-6,7-dihydro-5H-pyrido[3,4-d]pyrimidin-8-one

The title compound was prepared analogously as described in Example DA1 using 4-(3-Methanesulfonyl-phenyl)-5,6,7,8-tetrahydro-pyrido[3,4-d]pyrimidine instead of 3-trifluoromethyl-5,6,7,8-tetrahydro-[1,2,4]triazolo[4,3-a]pyrazine.

MS (ES⁺): 525 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.76 min.

### Example DA21

### 3-{6-[cis-4-Aminomethyl-4-(3-chloro-phenyl)-cyclohexyl]-5-oxo-5,6,7,8-tetrahydro-pyrido[4,3-d]pyrimidin-2-yl}-benzoic acid dihydrochloride

The title compound was prepared analogously as described in Example DA1 using 3-(5,6,7,8-Tetrahydro-pyrido[4,3-d]pyrimidin-2-yl)-benzoic acid ethyl ester instead of 3-trifluoromethyl-5,6,7,8-tetrahydro-[1,2,4]triazolo[4,3-a]pyrazine to afford 3-{6-[cis-4-Aminomethyl-4-(3-chloro-phenyl)-cyclohexyl]-5-oxo-5,6,7,8-tetrahydro-pyrido[4,3-d]pyrimidin-2-yl}-benzoic acid ethyl ester followed by step:

### K) 3-{6-[cis-4-Aminomethyl-4-(3-chloro-phenyl)-cyclohexyl]-5-oxo-5,6,7,8-tetrahydro-pyrido[4,3-d]pyrimidin-2-yl}-benzoic acid dihydrochloride

Lithiumhydroxide (41.7 mg, 0.98 mmol) was added to a mixture of 3-{6-[cis-4-Aminomethyl-4-(3-chloro-phenyl)-cyclohexyl]-5-oxo-5,6,7,8-tetrahydro-pyrido[4,3-d]pyrimidin-2-yl}-benzoic acid ethyl ester (56.3 mg, 0.098 mmol) in dioxane (0.8 ml) and water (0.8 ml) and the reaction was stirred at room temperature for 2 h. The reaction mixture was directly purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were lyophilized in vacuo to give the trifluoroacetate of the title compound, which was dissolved in acetonitril and water and treated with an excess of 1M hydrogen chloride in water (150 ul, 0.15 mmol). Removal of the volatiles by lyophilization gave the title compound as a white solid.

MS (ES⁺): 491 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 4.23 min.

### Example DA22

### 3-{7-[cis-4-Aminomethyl-4-(3-chloro-phenyl)-cyclohexyl]-8-oxo-5,6,7,8-tetrahydro-pyrido[3,4-d]pyrimidin-4-yl}-benzoic acid dihydrochloride

The title compound was prepared analogously as described in Example DA1 and DA2 using 3-(5,6,7,8-Tetrahydro-pyrido[3,4-d]pyrimidin-4-yl)-benzoic acid ethyl ester instead of 3-trifluoromethyl-5,6,7,8-tetrahydro-[1,2,4]triazolo[4,3-a]pyrazine.

MS (ES⁺): 491 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.75 min.

### Example DA23

### 7-[cis-4-Aminomethyl-4-(3-chloro-phenyl)-cyclohexyl]-6,7-dihydro-5H-pyrido[3,4-d]pyrimidin-8-one

The title compound was prepared analogously as described in Example DA1 using 5,6,7,8-Tetrahydro-pyrido[3,4-d]pyrimidine instead of 3-trifluoromethyl-5,6,7,8-tetrahydro-[1,2,4]triazolo[4,3-a]pyrazine.

MS (ES⁺): 371 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.42 min.

### Example DA24

### 6-[cis-4-Aminomethyl-4-(3-chloro-phenyl)-cyclohexyl]-2-(3-methanesulfonyl-phenyl)-7,8-dihydro-6H-pyrido[4,3-d]pyrimidin-5-one hydrochloride

The title compound was prepared analogously as described in Example DA1 using 2-(3-Methanesulfonyl-phenyl)-5,6,7,8-tetrahydro-pyrido[4,3-d]pyrimidine instead of 3-trifluoromethyl-5,6,7,8-tetrahydro-[1,2,4]triazolo[4,3-a]pyrazine.

MS (ES⁺): 525 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 4.34 min.

### Example DA25

### 34-{6-[cis-4-Aminomethyl-4-(3-chloro-phenyl)-cyclohexyl]-5-oxo-5,6,7,8-tetrahydro-pyrido[4,3-d]pyrimidin-2-yl}-N-methyl-benzenesulfonamide hydrochloride

The title compound was prepared analogously as described in Example DA1 using N-Methyl-3-(5,6,7,8-tetrahydro-pyrido[4,3-d]pyrimidin-2-yl)-benzenesulfonamide instead of 3-trifluoromethyl-5,6,7,8-tetrahydro-[1,2,4]triazolo[4,3-a]pyrazine.

MS (ES⁺): 540 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 4.37 min.

### Example DA26

### N-(3-{6-[cis-4-Aminomethyl-4-(3-chloro-phenyl)-cyclohexyl]-5-oxo-5,6,7,8-tetrahydro-pyrido[4,3-d]pyrimidin-2-yl}-phenyl)-methanesulfonamide hydrochloride

The title compound was prepared analogously as described in Example DA1 using N-[3-(5,6,7,8-Tetrahydro-pyrido[4,3-d]pyrimidin-2-yl)-phenyl]-methanesulfonamide instead of 3-trifluoromethyl-5,6,7,8-tetrahydro-[1,2,4]triazolo[4,3-a]pyrazine.

MS (ES⁺): 540 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 4.28 min.

### Example DA27

### N-(3-{7-[cis-4-Aminomethyl-4-(3-chloro-phenyl)-cyclohexyl]-8-oxo-5,6,7,8-tetrahydro-pyrido[3,4-d]pyrimidin-4-yl}-phenyl)-methanesulfonamide hydrochloride

The title compound was prepared analogously as described in Example DA1 using N-[3-(5,6,7,8-Tetrahydro-pyrido[3,4-d]pyrimidin-4-yl)-phenyl]-methanesulfonamide instead of 3-trifluoromethyl-5,6,7,8-tetrahydro-[1,2,4]triazolo[4,3-a]pyrazine.

MS (ES⁺): 540 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.82 min.

### Example DA28

### 7-[cis-4-Aminomethyl-4-(3-chloro-phenyl)-cyclohexyl]-4-(5-methyl-[1,2,4]oxadiazol-3-yl)-6,7-dihydro-5H-pyrido[3,4-d]pyrimidin-8-one

The title compound was prepared analogously as described in Example DA1 using 4-(5-methyl-[1,2,4]oxadiazol-3-yl)-5,6,7,8-tetrahydro-pyrido[3,4-d]pyrimidine instead of 3-trifluoromethyl-5,6,7,8-tetrahydro-[1,2,4]triazolo[4,3-a]pyrazine.

MS (ES⁺): 453 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.74 min.

### Example DA29

### 7-[cis-4-Aminomethyl-4-(3-chloro-phenyl)-cyclohexyl]-3,5,6,7-tetrahydro-pyrido[3,4-d]pyrimidine-4,8-dione hydrochloride

The title compound was prepared analogously as described in Example DA1 using 5,6,7,8-Tetrahydro-3H-pyrido[3,4-d]pyrimidin-4-one instead of 3-trifluoromethyl-5,6,7,8-tetrahydro-[1,2,4]triazolo[4,3-a]pyrazine.

MS (ES⁺): 387 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.31 min.

### Example DA30

### 7-[cis-4-Aminomethyl-4-(3-chloro-phenyl)-cyclohexyl]-4-oxo-3,4-dihydro-pyrido[3,4-d]pyrimidin-7-ium chloride

The title compound was prepared analogously as described in Example DA1 using 5,6,7,8-Tetrahydro-3H-pyrido[3,4-d]pyrimidin-4-one instead of 3-trifluoromethyl-5,6,7,8-tetrahydro-[1,2,4]triazolo[4,3-a]pyrazine.

MS (ES⁺): 369 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.03 min.

### Example DA31

### 6-[cis-4-Aminomethyl-4-(3-chloro-phenyl)-cyclohexyl]-5-oxo-5,6,7,8-tetrahydro-pyrido[4,3-d]pyrimidine-2-carboxylic acid amide trifluoroacetate

The title compound was prepared analogously as described in Example DA1 using 5,6,7,8-Tetrahydro-pyrido[4,3-d]pyrimidine-2-carboxylic acid amide instead of 3-trifluoromethyl-5,6,7,8-tetrahydro-[1,2,4]triazolo[4,3-a]pyrazine.

MS (ES⁺): 414 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.40 min.

### Example DB1

### 1-[cis-4-Aminomethyl-4-(3-chloro-phenyl)-cyclohexyl]-pyrrolidin-2-one hydrochloride

The title compound was prepared analogously as described in Example D1 step A to H, using Methyl-4-aminobutyrate hydrochloride instead of 3-trifluoromethyl-5,6,7,8-tetrahydro-[1,2,4]triazolo[4,3-a]pyrazine to afford 4-[4-(tert-Butoxycarbonylamino-methyl)-4-(cis-3-chloro-phenyl)-cyclohexylamino]-butyric acid methyl ester and 4-[4-(tert-Butoxycarbonylamino-methyl)-4-(trans-3-chloro-phenyl)-cyclohexylamino]-butyric acid methyl ester followed by step

### I) 1-[cis-4-Aminomethyl-4-(3-chloro-phenyl)-cyclohexyl]-pyrrolidin-2-one hydrochloride

To a solution of 4-[4-(tert-Butoxycarbonylamino-methyl)-4-(cis-3-chloro-phenyl)-cyclohexylamino]-butyric acid methyl ester (80 mg, 0.182 mmol) in Dimethylformamide (3 ml) was added Cesiumcarbonate (29 mg, 0.912 mmol). The mixture was stirred for 16 hours at 80° C., then treated with microwave at 150° C. for 45 min. The reaction mixture was treated with aqueous Sodium bicarbonate solution (conc.) The product was extracted into dichloromethane. The combined organic extracts were dried over magnesium sulfate. The filtrate was concentrated in vacuo and the residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were lyophilized in vacuo to give the formate salt of the title compound, which was dissolved in methanol and treated with an excess of 2M hydrogen chloride in methanol. Removal of the volatiles gave the title compound as a white solid.

MS (ES⁺): 307 [M+H]⁺.

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 5.02 min.

### Example DB2

### 1-[trans-4-Aminomethyl-4-(3-chloro-phenyl)-cyclohexyl]-tetrahydro-pyrimidin-2-one hydrochloride

The title compound was prepared analogously as described in Example DB1, using (3-Amino-propyl)-carbamic acid benzyl ester instead of Methyl-4-aminobutyrate hydrochloride, step I from {3-[4-(tert-Butoxycarbonylamino-methyl)-4-(trans-3-chloro-phenyl)-cyclohexylamino]-propyl}-carbamic acid benzyl ester.

MS (ES⁺): 322[M+H]⁺.

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 5.23 min.

### Example DB3

### 1-[cis-4-Aminomethyl-4-(3-chloro-phenyl)-cyclohexyl]-tetrahydro-pyrimidin-2-one hydrochloride

The title compound was prepared analogously as described in Example DB1, using (3-Amino-propyl)-carbamic acid benzyl ester instead of Methyl-4-aminobutyrate hydrochloride.

MS (ES⁺): 322[M+H]⁺.

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 5.11 min.

### Example DB4

### 1-[trans-4-Aminomethyl-4-(3-chloro-phenyl)-cyclohexyl]-imidazolidin-2-one hydrochloride

The title compound was prepared analogously as described in Example DB1, using (2-Amino-ethyl)-carbamic acid benzyl ester instead of Methyl-4-aminobutyrate hydrochloride.

MS (ES⁺): 308[M+H]⁺.

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 5.21 min.

### Example DB5

### 1-[cis-4-Aminomethyl-4-(3-chloro-phenyl)-cyclohexyl]-imidazolidin-2-one hydrochloride

The title compound was prepared analogously as described in Example DB1, using (2-Amino-ethyl)-carbamic acid benzyl ester instead of Methyl-4-aminobutyrate hydrochloride.

MS (ES⁺): 308[M+H]⁺.

HPLC (Macherey-Nagel LiChrospher 100-5 RP18 Flow 1.5 mL/min, 12 min method (0-1.5 min 10% ACN. 1.5-6.5 min 10-100% ACN, 6.5-9.0 min 100% ACN, 9.0-12.0 min 100-10% ACN): 5.17 min.

### Example DC1

### 3-[cis-4-Aminomethyl-4-(3-chloro-phenyl)-cyclohexyl]-oxazolidin-2-one hydrochloride

The title compound was prepared analogously as described in Example D1 step A to H, using 1-Amino-2-ethanol instead of 3-trifluoromethyl-5,6,7,8-tetrahydro-[1,2,4]triazolo[4,3-a]pyrazine to afford [1-(cis-3-Chloro-phenyl)-4-(2-hydroxy-ethylamino)-cyclohexylmethyl]-carbamic acid tert-butyl ester and [1-(trans-3-Chloro-phenyl)-4-(2-hydroxy-ethylamino)-cyclohexylmethyl]-carbamic acid tert-butyl ester followed by step

### I) [1-(cis-3-Chloro-phenyl)-4-(2-oxo-oxazolidin-3-yl)-cyclohexylmethyl]-carbamic acid tert-butyl ester

To a solution of [1-(cis-3-Chloro-phenyl)-4-(2-hydroxy-ethylamino)-cyclohexylmethyl]-carbamic acid tert-butyl ester (103 mg, 0.269 mmol) in Dichloromethane (10 ml) was added N—N′-Carbonyldiimidazole (69 mg, 0.404 mmol) and Triethylamine (39 μL, 0.282 mmol). The mixture was stirred for 16 hours at room temperature. The reaction mixture was treated with 1N Hydrochloric acid. The product was extracted into dichloromethane. The combined organic extracts were dried over magnesium sulfate. The filtrate was concentrated in vacuo and the residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were lyophilized in vacuo to give the title compound as a white solid.

### J) 3-[cis-4-Aminomethyl-4-(3-chloro-phenyl)-cyclohexyl]-oxazolidin-2-one hydrochloride

Trifluoroacetic acid (1 mL) was added to a solution of [1-(cis-3-Chloro-phenyl)-4-(2-oxo-oxazolidin-3-yl)-cyclohexylmethyl]-carbamic acid tert-butyl ester (78 mg, 0.191 mmol) in dichloromethane (2 mL) and the reaction stirred at room temperature for 4 h. The reaction mixture was concentrated in vacuo and the residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were lyophilized in vacuo to give the formate salt of the title compound, which was dissolved in methanol and treated with an excess of 2M hydrogen chloride in methanol. Removal of the volatiles gave the title compound as a white solid.

MS (ES⁺): 309 [M+H]⁺.

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 4.98 min.

### Example DC2

### 3-[trans-4-Aminomethyl-4-(3-chloro-phenyl)-cyclohexyl]-[1,3]oxazinan-2-one hydrochloride

The title compound was prepared analogously as described in Example DC1, using 1-Amino-3-propanol instead of 1-Amino-2-ethanol, step I from [1-(trans-3-Chloro-phenyl)-4-(3-hydroxy-propylamino)-cyclohexylmethyl]-carbamic acid tert-butyl ester.

MS (ES⁺): 323 [M+H]⁺.

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 5.06 min.

### Example DC3

### 3-[cis-4-Aminomethyl-4-(3-chloro-phenyl)-cyclohexyl]-[1,3]oxazinan-2-one hydrochloride

The title compound was prepared analogously as described in Example DC1, using 1-Amino-3-propanol instead of 1-Amino-2-ethanol.

MS (ES⁺): 323 [M+H]⁺.

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 4.97 min.

### Example DC4

### (S)-3-[cis-4-Aminomethyl-4-(3-chloro-phenyl)-cyclohexyl]-4-methyl-oxazolidin-2-one hydrochloride

The title compound was prepared analogously as described in Example DC1, using (S)-2-Amino-propan-1-ol instead of 1-Amino-2-ethanol.

MS (ES⁺): 323 [M+H]⁺.

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 3.93 min.

### Example DC5

### (R)-3-[cis-4-Aminomethyl-4-(3-chloro-phenyl)-cyclohexyl]-4-methyl-oxazolidin-2-one hydrochloride

The title compound was prepared analogously as described in Example DC1, using (R)-2-Amino-propan-1-ol instead of 1-Amino-2-ethanol.

MS (ES⁺): 323 [M+H]⁺.

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 3.96 min.

### Example DC6

### (S)-3-[cis-4-Aminomethyl-4-(3-chloro-phenyl)-cyclohexyl]-5-methyl-oxazolidin-2-one hydrochloride

The title compound was prepared analogously as described in Example DC1, using (S)-1-Amino-propan-2-ol instead of 1-Amino-2-ethanol.

MS (ES⁺): 323 [M+H]⁺.

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 3.79 min.

### Example DC7

### (R)-3-[cis-4-Aminomethyl-4-(3-chloro-phenyl)-cyclohexyl]-5-methyl-oxazolidin-2-one hydrochloride

The title compound was prepared analogously as described in Example DC1, using (R)-1-Amino-propan-2-ol instead of 1-Amino-2-ethanol.

MS (ES⁺): 323 [M+H]⁺.

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 3.81 min.

### Example DC8

### (S)-3-[cis-4-Aminomethyl-4-(3-chloro-phenyl)-cyclohexyl]-4-phenyl-oxazolidin-2-one hydrochloride

The title compound was prepared analogously as described in Example DC1, using (S)-2-Amino-2-phenylethanol instead of 1-Amino-2-ethanol.

MS (ES⁺): 385 [M+H]⁺.

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 4.48 min.

### Example DC9

### (R)-3-[cis-4-Aminomethyl-4-(3-chloro-phenyl)-cyclohexyl]-4-phenyl-oxazolidin-2-one hydrochloride

The title compound was prepared analogously as described in Example DC1, using (R)-2-Amino-2-phenylethanol instead of 1-Amino-2-ethanol.

MS (ES⁺): 385 [M+H]⁺.

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 4.47 min.

### Example DC10

### (S)-3-[cis-4-Aminomethyl-4-(3-chloro-phenyl)-cyclohexyl]-5-phenyl-oxazolidin-2-one hydrochloride

The title compound was prepared analogously as described in Example DC1, using (S)-2-Amino-1-phenylethanol instead of 1-Amino-2-ethanol.

MS (ES⁺): 385 [M+H]⁺.

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 4.37 min.

### Example DC11

### (R)-3-[cis-4-Aminomethyl-4-(3-chloro-phenyl)-cyclohexyl]-5-phenyl-oxazolidin-2-one hydrochloride

The title compound was prepared analogously as described in Example DC1, using (R)-2-Amino-1-phenylethanol instead of 1-Amino-2-ethanol.

MS (ES⁺): 385 [M+H]⁺.

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 4.36 min.

### Example DC12

### (S)-3-[cis-4-Aminomethyl-4-(3-chloro-phenyl)-cyclohexyl]-4-isopropyl-oxazolidin-2-one hydrochloride

The title compound was prepared analogously as described in Example DC1, using (S)-2-Amino-3-methyl-butan-1-ol instead of 1-Amino-2-ethanol.

MS (ES⁺): 351 [M+H]⁺.

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 4.34 min.

### Example DC13

### (R)-3-[cis-4-Aminomethyl-4-(3-chloro-phenyl)-cyclohexyl]-4-isopropyl-oxazolidin-2-one hydrochloride

The title compound was prepared analogously as described in Example DC1, using (R)-2-Amino-3-methyl-butan-1-ol instead of 1-Amino-2-ethanol.

MS (ES⁺): 351 [M+H]⁺.

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 4.33 min.

### Example DC14

### (S)-3-[cis-4-Aminomethyl-4-(3-chloro-phenyl)-cyclohexyl]-4-benzyl-oxazolidin-2-one hydrochloride

The title compound was prepared analogously as described in Example DC1, using (S)-2-Amino-3-phenyl-propan-1-ol instead of 1-Amino-2-ethanol.

MS (ES⁺): 399 [M+H]⁺.

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 4.62 min.

### Example DC15

### (R)-3-[cis-4-Aminomethyl-4-(3-chloro-phenyl)-cyclohexyl]-4-benzyl-oxazolidin-2-one hydrochloride

The title compound was prepared analogously as described in Example DC1, using (R)-2-Amino-3-phenyl-propan-1-ol instead of 1-Amino-2-ethanol.

MS (ES⁺): 399 [M+H]⁺.

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 4.65 min.

### Example DC16

### 1-[cis-4-Aminomethyl-4-(3-chloro-phenyl)-cyclohexyl]-3-phenyl-imidazolid in-2-one hydrochloride

The title compound was prepared analogously as described in Example DC1, using N-Phenylethylenediamine instead of 1-Amino-2-ethanol.

MS (ES⁺): 384 [M+H]⁺.

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 4.49 min.

### Example DC17

### 1-[cis-4-Aminomethyl-4-(3-chloro-phenyl)-cyclohexyl]-3-(4-cyclopentylmethoxy-phenyl)-imidazolidin-2-one hydrochloride

The title compound was prepared analogously as described in Example DC1, using N-(4-Cyclopentylmethoxyphenyl)-ethylenediamine instead of 1-Amino-2-ethanol.

MS (ES⁺): 482 [M+H]⁺.

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 5.38 min.

### Example DC18

### 1-[cis-4-Aminomethyl-4-(3-chloro-phenyl)-cyclohexyl]-3-methyl-imidazolidin-2-one hydrochloride

The title compound was prepared analogously as described in Example DC1, using N-Methylethylenediamine instead of 1-Amino-2-ethanol.

MS (ES⁺): 322[M+H]⁺.

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 3.71 min.

### Example DC19

### 1-[cis-4-Aminomethyl-4-(3-chloro-phenyl)-cyclohexyl]-3-butyl-imidazolidin-2-one hydrochloride

The title compound was prepared analogously as described in Example DC1, using N-Butylethylenediamine instead of 1-Amino-2-ethanol.

MS (ES⁺): 364[M+H]⁺.

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 4.32 min.

### Example DC20

### 1-[cis-4-Aminomethyl-4-(3-chloro-phenyl)-cyclohexyl]-3-benzyl-imidazolidin-2-one hydrochloride

The title compound was prepared analogously as described in Example DC1, using N-Benzylethylenediamine instead of 1-Amino-2-ethanol.

MS (ES⁺): 398[M+H]⁺.

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 4.42 min.

### Example DD1

### N-[cis-4-Aminomethyl-4-(3-chloro-phenyl)-cyclohexyl]-N-cyclopropylmethyl-5-phenyl-nicotinamide hydrochloride

The title compound was prepared analogously as described in Example D1 step A to H, using Cyclopropanemethylamine instead of 3-trifluoromethyl-5,6,7,8-tetrahydro-[1,2,4]triazolo[4,3-a]pyrazine to afford [1-(cis-3-Chloro-phenyl)-4-(cyclopropylmethyl-amino)-cyclohexylmethyl]-carbamic acid tert-butyl ester and [1-(trans-3-Chloro-phenyl)-4-(cyclopropylmethyl-amino)-cyclohexylmethyl]-carbamic acid tert-butyl ester followed by step

### I) {1-(cis-3-Chloro-phenyl)-4-[cyclopropylmethyl-(5-phenyl-pyridine-3-carbonyl)-amino]-cyclohexylmethyl}-carbamic acid tert-butyl ester

To a solution of [1-(cis-3-Chloro-phenyl)-4-(cyclopropylmethyl-amino)-cyclohexylmethyl]-carbamic acid tert-butyl ester (40 mg, 0.102 mmol) and 5-Phenylnicotinic acid (28 mg, 0.132 mmol) in Dimethylformamide (1 ml) was added 0-(7-azabenzotriazol-1-yl)-N,N,N′,N′-tetramethyluronium hexafluorophosphate (59 mg, 0.153 mmol) and diisopropylethylamine (71 μL, 0.407 mmol). The mixture stirred at room temperature for one hour. The reaction mixture was directly purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were lyophilized in vacuo to give the title compound as a white solid.

MS (ES⁺): 574 [M+H]⁺.

### J) N-[cis-4-Aminomethyl-4-(3-chloro-phenyl)-cyclohexyl]-N-cyclopropylmethyl-5-phenyl-nicotinamide hydrochloride

To {1-(cis-3-Chloro-phenyl)-4-[cyclopropylmethyl-(5-phenyl-pyridine-3-carbonyl)-amino]-cyclohexylmethyl}-carbamic acid tert-butyl ester (56 mg, 0.098 mmol) was added 4N hydrogen chloride solution in dioxane (10 ml). The reaction mixture stirred at room temperature for one hour, then it was concentrated in vacuo. The residue was treated with diethyl ether in ultrasonic bath. The etheric phase was removed with a pipette. The residue was lyophilized in vacuo to give the title compound as white crystals.

MS (ES⁺): 474 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 4.50 min.

### Example DD2

### N-[cis-4-Aminomethyl-4-(3-chloro-phenyl)-cyclohexyl]-N-cyclopropyl-5-phenyl-nicotinamide hydrochloride

The title compound was prepared analogously as described in Example DD1 using Cyclopropylamine instead of Cyclopropanemethylamine.

MS (ES⁺): 460 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 4.33 min.

### Example DD3

### N-[cis-4-Aminomethyl-4-(3-chloro-phenyl)-cyclohexyl]-N-(2-methoxy-ethyl)-5-phenyl-nicotinamide hydrochloride

The title compound was prepared analogously as described in Example DD1 using 2-Methoxyethylamine instead of Cyclopropanemethylamine.

MS (ES⁺): 478 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 4.24 min.

### Example DD4

### N-[cis-4-Aminomethyl-4-(3-chloro-phenyl)-cyclohexyl]-N-methylcarbamoylmethyl-5-phenyl-nicotinamide hydrochloride

The title compound was prepared analogously as described in Example DD1 using 2-Amino-N-methylacetamide hydrochloride instead of Cyclopropanemethylamine.

MS (ES⁺): 491 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.97 min.

### Example DD5

### 6-Acetylamino-N-[cis-4-aminomethyl-4-(3-chloro-phenyl)-cyclohexyl]-N-cyclopropylmethyl-nicotinamide hydrochloride

The title compound was prepared analogously as described in Example DD1 using 6-Acetylaminonicotinic acid instead of 5-Phenylnicotinic acid.

MS (ES⁺): 455 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.96 min.

### Example DD6

### 6-Acetylamino-N-[cis-4-aminomethyl-4-(3-chloro-phenyl)-cyclohexyl]-N-cyclopropyl-nicotinamide hydrochloride

The title compound was prepared analogously as described in Example DD1 using Cyclopropylamine instead of Cyclopropanemethylamine and 6-Acetylaminonicotinic acid instead of 5-Phenylnicotinic acid.

MS (ES⁺): 441 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.81 min.

### Example DD7

### 6-Acetylamino-N-[cis-4-aminomethyl-4-(3-chloro-phenyl)-cyclohexyl]-N-(2-methoxy-ethyl)-nicotinamide hydrochloride

The title compound was prepared analogously as described in Example DD1 using 2-Methoxyethylamine instead of Cyclopropanemethylamine and 6-Acetylaminonicotinic acid instead of 5-Phenylnicotinic acid.

MS (ES⁺): 459 [M+H]⁺.

### Example DD8

### 6-Acetylamino-N-[cis-4-aminomethyl-4-(3-chloro-phenyl)-cyclohexyl]-N-methylcarbamoylmethyl-nicotinamide hydrochloride

The title compound was prepared analogously as described in Example DD1 using 2-Amino-N-methylacetamide hydrochloride instead of Cyclopropanemethylamine and 6-Acetylaminonicotinic acid instead of 5-Phenylnicotinic acid.

MS (ES⁺): 472 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.34 min.

### Example DD9

### Pyridazine-3-carboxylic acid [cis-4-aminomethyl-4-(3-chloro-phenyl)-cyclohexyl]-cyclopropyl-amide hydrochloride

The title compound was prepared analogously as described in Example DD1 using Cyclopropylamine instead of Cyclopropanemethylamine and Pyridazine-3-carboxylic acid instead of 5-Phenylnicotinic acid.

MS (ES⁺): 385 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.76 min.

### Example DD10

### Pyridazine-3-carboxylic acid [cis-4-aminomethyl-4-(3-chloro-phenyl)-cyclohexyl]-(2-methoxy-ethyl)-amide hydrochloride

The title compound was prepared analogously as described in Example DD1 using 2-Methoxyethylamine instead of Cyclopropanemethylamine and Pyridazine-3-carboxylic acid instead of 5-Phenylnicotinic acid.

MS (ES⁺): 403 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.60 min.

### Example DD11

### Pyridazine-3-carboxylic acid [cis-4-aminomethyl-4-(3-chloro-phenyl)-cyclohexyl]-methylcarbamoylmethyl-amide hydrochloride

The title compound was prepared analogously as described in Example DD1 using 2-Amino-N-methylacetamide hydrochloride instead of Cyclopropanemethylamine and Pyridazine-3-carboxylic acid instead of 5-Phenylnicotinic acid.

MS (ES⁺): 416 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.30 min.

### Example DD12

### 1-Isopropyl-1H-benzotriazole-5-carboxylic acid [cis-4-aminomethyl-4-(3-chloro-phenyl)-cyclohexyl]-cyclopropylmethylamide hydrochloride

The title compound was prepared analogously as described in Example DD1 using 1-Isopropyl-1H-1,2,3-benzotriazole-5-carboxylic acid instead of 5-Phenylnicotinic acid.

MS (ES⁺): 480 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 4.57 min.

### Example DD13

### 1-Isopropyl-1H-benzotriazole-5-carboxylic acid [cis-4-aminomethyl-4-(3-chloro-phenyl)-cyclohexyl]-cyclopropylamide hydrochloride

The title compound was prepared analogously as described in Example DD1 using Cyclopropylamine instead of Cyclopropanemethylamine and 1-Isopropyl-1H-1,2,3-benzotriazole-5-carboxylic acid instead of 5-Phenylnicotinic acid.

MS (ES⁺): 466 [M+H]⁺.

HPLC (Nucleosil C-18HD 4×70 mm 3 μm, 8 min method (0-6 min 20-100% ACN, 6.0-7.5 min 100% ACN, 7.5-8.0 min 100-20% ACN): 2.49 min.

### Example DD14

### 1-Isopropyl-1H-benzotriazole-5-carboxylic acid [cis-4-aminomethyl-4-(3-chloro-phenyl)-cyclohexyl]-(2-methoxy-ethyl)-amide hydrochloride

The title compound was prepared analogously as described in Example DD1 using 2-Methoxyethylamine instead of Cyclopropanemethylamine and 1-Isopropyl-1H-1,2,3-benzotriazole-5-carboxylic acid instead of 5-Phenylnicotinic acid.

MS (ES⁺): 484 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 4.25 min.

### Example DD15

### 1-Isopropyl-1H-pyrazolo[3,4-b]pyridine-5-carboxylic acid [cis-4-aminomethyl-4-(3-chloro-phenyl)-cyclohexyl]-cyclopropylmethyl-amide hydrochloride

The title compound was prepared analogously as described in Example DD1 using 1-isopropyl-1H-pyrazolo[3,4-b]-pyridine-5-carboxylic acid instead of 5-Phenylnicotinic acid.

MS (ES⁺): 480 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 4.65 min.

### Example DD16

### 6-Amino-N-[cis-4-aminomethyl-4-(3-chloro-phenyl)-cyclohexyl]-N-cyclopropylmethyl-nicotinamide hydrochloride

The title compound was prepared analogously as described in Example DD1 using 6-Aminonicotinic acid instead of 5-Phenylnicotinic acid.

MS (ES⁺): 413 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.57 min.

### Example DD17

### N-[cis-4-Aminomethyl-4-(3-chloro-phenyl)-cyclohexyl]-N-cyclopropylmethyl-isonicotinamide hydrochloride

The title compound was prepared analogously as described in Example DD1 using Isonicotinic acid instead of 5-Phenylnicotinic acid.

MS (ES⁺): 398 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.56 min.

### Example DD18

### N-[cis-4-Aminomethyl-4-(3-chloro-phenyl)-cyclohexyl]-N-cyclopropylmethyl-nicotinamide hydrochloride

The title compound was prepared analogously as described in Example DD1 using Nicotinic acid instead of 5-Phenylnicotinic acid.

MS (ES⁺): 398 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.67 min.

### Example DD19

### N-[cis-4-Aminomethyl-4-(3-chloro-phenyl)-cyclohexyl]-N-(2-methanesulfonyl-ethyl)-nicotinamide hydrochloride

The title compound was prepared analogously as described in Example DD1 using 2-Methanesulfonyl-ethylamine instead of Cyclopropanemethylamine and Nicotinic acid instead of 5-Phenylnicotinic acid.

MS (ES⁺): 450 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.17 min.

### Example DD₂O

### [[cis-4-Aminomethyl-4-(3-chloro-phenyl)-cyclohexyl]-(pyridine-3-carbonyl)-amino]-acetic acid hydrochloride

The title compound was prepared analogously as described in Example DD1 using Amino-acetic acid tert-butyl ester hydrochloride instead of Cyclopropanemethylamine and Nicotinic acid instead of 5-Phenylnicotinic acid.

MS (ES⁺): 402 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.11 min.

### Example DD21

### N-[cis-4-Aminomethyl-4-(3-chloro-phenyl)-cyclohexyl]-N-(3H-imidazol-4-ylmethyl)-nicotinamide hydrochloride

The title compound was prepared analogously as described in Example DD1 using C-(3H-Imidazol-4-yl)-methylamine hydrochloride instead of Cyclopropanemethylamine and Nicotinic acid instead of 5-Phenylnicotinic acid.

MS (ES⁺): 424 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.09 min.

### Example DD22

### 3-[[cis-4-Aminomethyl-4-(3-chloro-phenyl)-cyclohexyl]-(pyridine-3-carbonyl)-amino]-propionic acid ethyl ester hydrochloride

The title compound was prepared analogously as described in Example DD1 using Amino-propionic acid ethyl ester hydrochloride instead of Cyclopropanemethylamine and Nicotinic acid instead of 5-Phenylnicotinic acid.

MS (ES+): 444 [M+H]+.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.56 min.

### Example DD23

### N-[cis-4-Aminomethyl-4-(3-chloro-phenyl)-cyclohexyl]-N-(2-hydroxy-ethyl)-nicotinamide hydrochloride

The title compound was prepared analogously as described in Example DD1 using 2-Aminoethanol hydrochloride instead of Cyclopropanemethylamine and Nicotinic acid instead of 5-Phenylnicotinic acid.

MS (ES+): 388 [M+H]+.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.09 min.

### Example DD24

### 3-[[cis-4-Aminomethyl-4-(3-chloro-phenyl)-cyclohexyl]-(pyridine-3-carbonyl)-amino]-propionic acid hydrochloride

The title compound was prepared analogously as described in Example DD1 step A to I using Aminopropionic acid ethyl ester hydrochloride instead of Cyclopropanemethylamine and Nicotinic acid instead of 5-Phenylnicotinic acid followed by step

### J) 3-[[4-(tert-Butoxycarbonylamino-methyl)-4-(cis-3-chlorophenyl)-cyclohexyl]-pyridine-3-carbonyl)-amino]-propionic acid

To a solution of 3-[[4-(tert-Butoxycarbonylamino-methyl)-4-(cis-3-chlorophenyl)-cyclohexyl]-(pyridine-3-carbonyl)-amino]-propionic acid ethyl ester (55 mg, 0.101 mmol) in dioxane (0.5 ml) and water (0.15 ml) was added Lithium hydroxide (8.6 mg, 0.202 mmol). The mixture was stirred at 45° C. for one hour. The reaction mixture was treated with 2N Hydrochloric acid and was directly purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were lyophilized in vacuo to give the title compound as a white solid.

MS (ES⁺): 516 [M+H]⁺.

### K) 3-[[cis-4-Aminomethyl-4-(3-chloro-phenyl)-cyclohexyl]-(pyridine-3-carbonyl)-amino]-propionic acid hydrochloride

To {3-[[4-(tert-Butoxycarbonylamino-methyl)-4-(cis-3-chlorophenyl)-cyclohexyl]-(pyridine-3-carbonyl)-amino]-propionic acid (39 mg, 0.076 mmol) was added 4N hydrogen chloride solution in dioxane (5 ml). The reaction mixture stirred at room temperature for one hour, then it was concentrated in vacuo. The residue was treated with diethyl ether in ultrasonic bath. The etheric phase was removed with a pipette. The residue was lyophilized in vacuo to give the title compound as white crystals.

MS (ES⁺): 416 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.16 min.

### Example DD25

### N-[cis-4-Aminomethyl-4-(3-chloro-phenyl)-cyclohexyl]-N-(2H-pyrazol-3-ylmethyl)-nicotinamide hydrochloride

The title compound was prepared analogously as described in Example DC1 using 2-H-pyrazol-3-ylmethylamine dihydrochloride instead of Cyclopropanemethylamine and Nicotinic acid instead of 5-Phenylnicotinic acid.

MS (ES+): 424 [M+H]+.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.28 min.

### Example DD26

### N-[4-Aminomethyl-4-(3-chloro-phenyl)-cyclohexyl]-N-(1H-imidazol-2-ylmethyl)-nicotinamide hydrochloride

The title compound was prepared analogously as described in Example DC1 using 1-H-imidazol-2-ylmethylamine dihydrochloride instead of Cyclopropanemethylamine and Nicotinic acid instead of 5-Phenylnicotinic acid.

MS (ES+): 424 [M+H]+.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.93 min.

### Example DD27

### N-[cis-4-Aminomethyl-4-(3-chloro-phenyl)-cyclohexyl]-N-[2-(3-methanesulfonyl-phenyl)-ethyl]-nicotinamide hydrochloride

The title compound was prepared analogously as described in Example DC1 using 2-(3-Methanesulfonyl-phenyl)-ethylamine instead of Cyclopropanemethylamine and Nicotinic acid instead of 5-Phenylnicotinic acid.

MS (ES+): 526 [M+H]+.

HPLC (Nucleosil C-18HD 4×70 mm 3 μm, 8 min method (0-6 min 5-100% ACN, 6.0-7.5 min 100% ACN, 7.5-8.0 min 100-5% ACN): 2.50 min.

### Example DD28

### N-[cis-4-Aminomethyl-4-(3-chloro-phenyl)-cyclohexyl]-2,2,2-trifluoro-N-[2-(3-methanesulfonyl-phenyl)-ethyl]-acetamide hydrochloride

The title compound was prepared analogously as described in Example DC1 using 2-(3-Methanesulfonyl-phenyl)-ethylamine instead of Cyclopropanemethylamine and Trifluoroacetic acid instead of 5-Phenylnicotinic acid.

MS (ES+): 517 [M+H]+.

HPLC (Nucleosil C-18HD 4×70 mm 3 μm, 8 min method (0-6 min 5-100% ACN, 6.0-7.5 min 100% ACN, 7.5-8.0 min 100-5% ACN): 2.39 min.

### Example DD29

### Tetrahydro-pyran-4-carboxylic acid [cis-4-aminomethyl-4-(3-chloro-phenyl)-cyclohexyl]-[2-(3-methanesulfonyl-phenyl)-ethyl]-amide hydrochloride

The title compound was prepared analogously as described in Example DC1 using 2-(3-Methanesulfonyl-phenyl)-ethylamine instead of Cyclopropanemethylamine and Tetrahydropyran-4-carboxylic acid instead of 5-Phenylnicotinic acid.

MS (ES+): 533 [M+H]+.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.42 min.

### Example DD30

### N-[cis-4-Aminomethyl-4-(3-chloro-phenyl)-cyclohexyl]-N-[2-(3-methanesulfonyl-phenyl)-ethyl]-3-methoxy-propionamide hydrochloride

The title compound was prepared analogously as described in Example DC1 using 2-(3-Methanesulfonyl-phenyl)-ethylamine instead of Cyclopropanemethylamine and 3-Methoxypropionic acid instead of 5-Phenylnicotinic acid.

MS (ES+): 507 [M+H]+.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.43 min.

### Example DD31

### N-[cis-4-Aminomethyl-4-(3-chloro-phenyl)-cyclohexyl]-N-[2-(3-methanesulfonyl-phenyl)-ethyl]-2-methoxy-acetamide hydrochloride

The title compound was prepared analogously as described in Example DC1 using 2-(3-Methanesulfonyl-phenyl)-ethylamine instead of Cyclopropanemethylamine and Methoxyacetic acid instead of 5-Phenylnicotinic acid.

MS (ES+): 493 [M+H]+.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.30 min.

### Example DD32

### Piperidine-4-carboxylic acid [cis-4-aminomethyl-4-(3-chloro-phenyl)-cyclohexyl]-[2-(3-methanesulfonyl-phenyl)-ethyl]-amide hydrochloride

The title compound was prepared analogously as described in Example DC1 using 2-(3-Methanesulfonyl-phenyl)-ethylamine instead of Cyclopropanemethylamine and Piperidine-1,4-dicarboxylic acid mono-tert-butyl ester instead of 5-Phenylnicotinic acid.

MS (ES+): 532 [M+H]+.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.57 min.

### Example DE1

### 1-[cis-4-Aminomethyl-4-(3-chloro-phenyl)-cyclohexyl]-piperazine-2,5-dione

The title compound was prepared analogously as described in Example D1 step A to H using (2-Amino-acetylamino)-acetic acid ethyl ester hydrochloride instead of 3-trifluoromethyl-5,6,7,8-tetrahydro-[1,2,4]triazolo[4,3-a]pyrazine to afford {2-[4-(tert-Butoxycarbonylamino-methyl)-4-(cis-3-chloro-phenyl)-cyclohexylamino]-acetylamino}-acetic acid ethyl ester and {2-[4-(tert-Butoxycarbonylamino-methyl)-4-(trans-3-chloro-phenyl)-cyclohexylamino]-acetylamino}-acetic acid ethyl ester followed by step

### I) [1-(cis-3-Chloro-phenyl)-4-(2,5-dioxo-piperazin-1-yl)-cyclohexylmethyl]-carbamic acid tert-butyl ester

{2-[4-(tert-Butoxycarbonylamino-methyl)-4-(cis-3-chloro-phenyl)-cyclohexylamino]-acetylamino}-acetic acid ethyl ester (128 mg, 0.252 mmol) was dissolved in a mixture of toluene (5 ml), n-Butanol (5 ml) and acetic acid (1 ml). The solution was heated in microwave at 150° C. for one hour, then the mixture was concentrated in vacuo to give the title compound as a white solid.

MS (ES⁺): 458 [M+Na]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 3.19 min.

### J) 1-[cis-4-Aminomethyl-4-(3-chloro-phenyl)-cyclohexyl]-piperazine-2,5-dione

Trifluoroacetic acid (0.4 mL) was added to a solution of [1-(cis-3-Chloro-phenyl)-4-(2,5-dioxo-piperazin-1-yl)-cyclohexylmethyl]-carbamic acid tert-butyl ester (87 mg, 0.180 mmol) in dichloromethane (4 mL) and the reaction was stirred at room temperature for 5 hours, then it was stirred at 40° C. for 6 hours. The reaction mixture was concentrated in vacuo and the residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were concentrated in vacuo, then partitioned between dichloromethane and saturated aqueous sodium bicarbonate solution. The organic layer was dried over sodium sulfate and concentrated in vacuo to give the title compound as a white solid.

MS (ES⁺): 336 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.58 min.

### Example DE2

### 1-[cis-4-Aminomethyl-4-(3-chloro-phenyl)-cyclohexyl]-4-phenyl-piperazin-2-one hydrochloride

The title compound was prepared analogously as described in Example DE1 using [(2-Amino-ethyl)-phenyl-amino]-acetic acid ethyl ester hydrochloride instead of (2-Amino-acetylamino)-acetic acid ethyl ester hydrochloride.

The reaction mixture of step J was concentrated in vacuo and the residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were lyophilized in vacuo to give the formate salt of the title compound, which was dissolved in methanol and treated with an excess of 2M hydrogen chloride in methanol. Removal of the volatiles gave the title compound as a white solid.

MS (ES⁺): 398 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 3.26 min.

### Example DE3

### (7R,8aS)-2-[trans-4-Aminomethyl-4-(3-chloro-phenyl)-cyclohexyl]-7-hydroxy-hexahydro-pyrrolo[1,2-a]pyrazine-1,4-dione formate

The title compound was prepared analogously as described in Example DE1 using (2S,4R)-1-(2-Amino-acetyl)-4-hydroxy-pyrrolidine-2-carboxylic acid methyl ester hydrochloride instead of (2-Amino-acetylamino)-acetic acid ethyl ester hydrochloride, step I from (2S,4R)-1-{2-[4-(tert-Butoxycarbonylamino-methyl)-4-(trans-3-chloro-phenyl)-cyclohexylamino]-acetyl}-4-hydroxy-pyrrolidine-2-carboxylic acid methyl ester.

The reaction mixture of step J was concentrated in vacuo and the residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were lyophilized in vacuo to give the title compound as a white solid.

MS (ES⁺): 392 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.38 min.

### Example DE4

### 1-[cis-4-Aminomethyl-4-(3-chloro-phenyl)-cyclohexyl]-4-phenyl-piperazine-2,5-dione

The title compound was prepared analogously as described in Example DE1 using [(2-Amino-acetyl)-phenyl-amino]-acetic acid ethyl ester hydrochloride instead of (2-Amino-acetylamino)-acetic acid ethyl ester hydrochloride.

MS (ES⁺): 412 [M+H]⁺.

HPLC (Waters Symmetry C18 3.5 μm 2.1×50 mm, 6 min method (0-3 min 20-95% ACN, 3.5-5.5 min 95% ACN, 5.5-5.55 min 95-5% ACN, 5.55-6 min 20% ACN): 2.41 min.

### Example DE5

### (7R,8aS)-2-[cis-4-Aminomethyl-4-(3-chloro-phenyl)-cyclohexyl]-7-hydroxy-hexahydro-pyrrolo[1,2-a]pyrazine-1,4-dione formate

The title compound was prepared analogously as described in Example DE1 using (2S,4R)-1-(2-Amino-acetyl)-4-hydroxy-pyrrolidine-2-carboxylic acid methyl ester hydrochloride instead of (2-Amino-acetylamino)-acetic acid ethyl ester hydrochloride.

The reaction mixture of step J was concentrated in vacuo and the residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were lyophilized in vacuo to give the title compound as a white solid.

MS (ES⁺): 392 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.56 min.

### Example DE6

### 3-{4-[4-Aminomethyl-4-(3-chloro-phenyl)-cyclohexyl]-2,5-dioxo-piperazin-1-yl}-benzoic acid formate

The title compound was prepared analogously as described in Example DE1 using 3-[(2-Amino-acetyl)-ethoxycarbonylmethyl-amino]-benzoic acid ethyl ester hydrochloride instead of (2-Amino-acetylamino)-acetic acid ethyl ester hydrochloride followed by step

### J) 3-{4-[4-(tert-Butoxycarbonylamino-methyl)-4-(3-chloro-phenyl)-cyclohexyl]-2,5-dioxo-piperazin-1-yl}-benzoic acid and 3-[({[4-(tert-Butoxycarbonylamino-methyl)-4-(3-chloro-phenyl)-cyclohexyl]-carboxymethyl-carbamoyl}-methyl)-amino]-benzoic acid

To a solution of 3-{4-[4-(tert-Butoxycarbonylamino-methyl)-4-(3-chloro-phenyl)-cyclohexyl]-2,5-dioxo-piperazin-1-yl}-benzoic acid ethyl ester (43 mg, 0.074 mmol) in tetrahydrofurane (1 ml) and water (1 ml) was added Lithium hydroxide (16 mg, 0.368 mmol). The mixture was stirred at 60° C. for 4 h. The reaction mixture was treated with 1N Hydrochloric acid and extracted into dichloromethane. The organic layer was dried over sodium sulfate and evaporated in vacuo to give a mixture of the title compounds as a white solid.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 3.60 min.

### H) 3-{4-[4-Aminomethyl-4-(3-chloro-phenyl)-cyclohexyl]-2,5-dioxo-piperazin-1-yl}-benzoic acid formate

To the solution of the mixture of 3-{4-[4-(tert-Butoxycarbonylamino-methyl)-4-(3-chloro-phenyl)-cyclohexyl]-2,5-dioxo-piperazin-1-yl}-benzoic acid and 3-[({[4-(tert-Butoxycarbonyl-amino-methyl)-4-(3-chloro-phenyl)-cyclohexyl]-carboxymethyl-carbamoyl}-methyl)-amino]-benzoic acid (38 mg, 0.068 mmol) in dichloromethane (2.5 ml) was added trifluoroacetic acid (0.4 mL). The reaction mixture stirred at room temperature for 2 h, then it was concentrated in vacuo. The residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the title compound were lyophilized in vacuo to give the title compound as a white solid.

MS (ES⁺): 456 [M+H]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 2.11 min.

### Example DE7

### (S)-1-[trans-4-Aminomethyl-4-(3-chloro-phenyl)-cyclohexyl]-3-benzyl-piperazine-2,5-dione trifluoroacetate

The title compound was prepared analogously as described in Example DE1 using (S)-2-(2-Amino-acetylamino)-3-phenyl-propionic acid methyl ester instead of (2-Amino-acetylamino)-acetic acid ethyl ester hydrochloride, step I from [4-((S)-3-Benzyl-2,5-dioxo-piperazin-1-yl)-1-(trans-3-chloro-phenyl)-cyclohexylmethyl]-carbamic acid tert-butyl ester.

The reaction mixture of step J was concentrated in vacuo and the residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were lyophilized in vacuo to give the title compound as a white solid.

MS (ES⁺): 426 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% AeN): 2.76 min.

### Example DE8

### (S)-1-[cis-4-Aminomethyl-4-(3-chloro-phenyl)-cyclohexyl]-3-benzyl-piperazine-2,5-dione formate

The title compound was prepared analogously as described in Example DE1 using (S)-2-(2-Amino-acetylamino)-3-phenyl-propionic acid methyl ester instead of (2-Amino-acetylamino)-acetic acid ethyl ester hydrochloride.

The reaction mixture of step J was concentrated in vacuo and the residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were lyophilized in vacuo to give the title compound as a white solid.

MS (ES⁺): 426 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.85 min.

### Example DE9

### (R)-2-[cis-4-Aminomethyl-4-(3-chloro-phenyl)-cyclohexyl]-hexahydro-pyrrolo[1,2-a]pyrazine-1,4-dione

The title compound was prepared analogously as described in Example DE1 using (R)-1-(2-Amino-acetyl)-pyrrolidine-2-carboxylic acid methyl ester hydrochloride instead of (2-Amino-acetylamino)-acetic acid ethyl ester hydrochloride.

MS (ES⁺): 376 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.73 min.

### Example DE10

### 3-[({[cis-4-Aminomethyl-4-(3-chloro-phenyl)-cyclohexyl]-carboxymethyl-carbamoyl}-methyl)-amino]-benzoic acid formate

The title compound was prepared analogously as described in Example DE6, isolating the title compound as a white solid during the prep. HPLC purification in step H.

MS (ES⁺): 474, 476 [M+H]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 2.34 min.

### Example DE11

### (S)-2-[4-Aminomethyl-4-(3-chloro-phenyl)-cyclohexyl]-hexahydro-pyrido[1,2-a]pyrazine-1,4-dione

The title compound was prepared analogously as described in Example DE1 using (S)-1-(2-Amino-acetyl)-piperidine-2-carboxylic acid methyl ester trifluoroacetate instead of (2-Amino-acetylamino)-acetic acid ethyl ester hydrochloride.

MS (ES⁺): 390 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.93 min.

### Example DF1

### 7-[cis-4-Aminomethyl-4-(3-chloro-phenyl)-cyclohexyl]-3-methyl-7,8-dihydro-[1,2,4]triazolo[4,3-a]pyrazin-6-one dihydrochloride

The title compound was prepared analogously as described in Example D1 step A to H using (2-Amino-acetylamino)-acetic acid ethyl ester hydrochloride instead of 3-trifluoromethyl-5,6,7,8-tetrahydro[1,2,4]triazolo[4,3-a]pyrazine to afford a mixture of {2-[4-(tert-Butoxycarbonylamino-methyl)-4-(cis-3-chloro-phenyl)-cyclohexylamino]-acetylamino}-acetic acid ethyl ester and {2-[4-(tert-Butoxycarbonylamino-methyl)-4-(trans-3-chloro-phenyl)-cyclohexylamino]-acetylamino}-acetic acid ethyl ester followed by step

### I) [1-(cis-3-Chloro-phenyl)-4-(2,5-dioxo-piperazin-1-yl)-cyclohexylmethyl]-carbamic acid tert-butyl ester and [1-(trans-3-Chloro-phenyl)-4-(2,5-dioxo-piperazin-1-yl)-cyclohexylmethyl]-carbamic acid tert-butyl ester

A mixture of {2-[4-(tert-Butoxycarbonylamino-methyl)-4-(cis-3-chloro-phenyl)-cyclohexylamino]-acetylamino}-acetic acid ethyl ester and {2-[4-(tert-Butoxycarbonylamino-methyl)-4-(trans-3-chloro-phenyl)-cyclohexylamino]-acetylamino}-acetic acid ethyl ester (437 mg, 0.907 mmol) was dissolved in a mixture of toluene (6 ml), n-Butanol (6 ml) and acetic acid (1.2 ml). The solution was stirred in a sealed tube at 170° C. for 2 h. The mixture was quenched with water and the product was extracted 3× into ethyl acetate. The combined organic fractions were dried over sodium sulfate and concentrated in vacuo to give the title compound as a white solid.

MS (ES⁺): 458 [MA-Na]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 3.01 min (trans) and 3.19 min (cis).

### J) [1-(cis-3-Chloro-phenyl)-4-(5-ethoxy-2-oxo-3,6-dihydro-2H-pyrazin-1-yl)-cyclohexylmethyl]-carbamic acid tert-butyl ester and 1-(trans-3-Chloro-phenyl)-4-(5-ethoxy-2-oxo-3,6-dihydro-2H-pyrazin-1-yl)-cyclohexylmethyl]-carbamic acid tert-butyl ester

To a suspension of a mixture of [1-(cis-3-Chloro-phenyl)-4-(2,5-dioxo-piperazin-1-yl)-cyclohexylmethyl]-carbamic acid tert-butyl ester and [1-(trans-3-Chloro-phenyl)-4-(2,5-dioxo-piperazin-1-yl)-cyclohexylmethyl]-carbamic acid tert-butyl ester (100 mg, 0.229 mmol) in dichloromethane (2 ml) were added Triethyloxonium tetrafluoroborate (1N in dichloromethane, 1.15 ml, 1.15 mmol) and anhydrous sodium carbonate (485 mg, 4.58 mmol) The reaction mixture was stirred at room temperature for 16 h. The mixture was quenched with water and the product was extracted 2× into dichloromethane. The combined organic fractions were dried over sodium sulfate and concentrated in vacuo to give the title compound as a yellow oil.

MS (ES⁺): 464 [M+H]⁺.

### K) [1-(cis-3-Chloro-phenyl)-4-(3-methyl-6-oxo-5,6-dihydro-8H-[1,2,4]triazolo[4,3-a]pyrazin-7-4-cyclohexylmethyl]-carbamic acid tert-butyl ester

To a solution of a mixture of [1-(cis-3-Chloro-phenyl)-4-(5-ethoxy-2-oxo-3,6-dihydro-2H-pyrazin-1-yl)-cyclohexylmethyl]-carbamic acid tert-butyl ester and 1-(trans-3-Chloro-phenyl)-4-(5-ethoxy-2-oxo-3,6-dihydro-2H-pyrazin-1-yl)-cyclohexylmethyl]-carbamic acid tert-butyl ester (55 mg, 0.115 mmol) in n-Butanol (1 ml) was added a solution of Acetic acid hydrazide (19 mg, 0.23 mmol) in n-Butanol (1 ml). The reaction mixture was refluxed for 5 h. The mixture was diluted with dichloromethane and washed with water and brine. The organic layer was dried over sodium sulfate and concentrated in vacuo. The residue was purified by prep. HPLC (Waters SunFire Prep C18 0 DB 5 μm 19×50 mm, flow 20 ml/min, 15 min method (0-2.5 min 5% ACN, 2.5-22.5 min 5-100% ACN, 22.5-25.0 min 100% ACN). Fractions containing the product were concentrated in vacuo to give the title compound as a white solid.

MS (ES⁺): 474 [M+H]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 3.09 min.

### L) 7-[cis-4-Aminomethyl-4-(3-chloro-phenyl)-cyclohexyl]-3-methyl-7,8-dihydro-[1,2,4]triazolo[4,3-a]pyrazin-6-one dihydrochloride

Trifluoroacetic acid (0.5 mL) was added to a solution of [1-(cis-3-Chloro-phenyl)-4-(3-methyl-6-oxo-5,6-dihydro-8H-[1,2,4]triazolo[4,3-a]pyrazin-7-yl)-cyclohexylmethyl]-carbamic acid tert-butyl ester (5 mg, 0.011 mmol) in dichloromethane (0.5 mL). The reaction mixture was stirred at room temperature for 10 minutes. The mixture was concentrated in vacuo to give the trifluoro acetate of the title compound, which was dissolved in methanol and treated with an excess of 2M hydrogen chloride in methanol. Removal of the volatiles gave the title compound as a white solid.

MS (ES⁺): 374 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.64 min.

### Example DF2

### 7-[trans-4-Aminomethyl-4-(3-chloro-phenyl)-cyclohexyl]-3-methyl-7,8-dihydro-[1,2,4]triazolo[4,3-a]pyrazin-6-one dihydrochloride

The title compound was prepared analogously as described in Example DF1, step L from [1-(trans-3-Chloro-phenyl)-4-(3-methyl-6-oxo-5,6-dihydro-8H-[1,2,4]triazolo[4,3-a]pyrazin-7-yl)-cyclohexylmethyl]-carbamic acid tert-butyl ester.

MS (ES⁺): 374 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.24 min.

### Example DF3

### 7-[cis-4-Aminomethyl-4-(3-chloro-phenyl)-cyclohexyl]-3-pyridin-4-yl-7,8-dihydro-[1,2,4]triazolo[4,3-a]pyrazin-6-one dihydrochloride

The title compound was prepared analogously as described in Example DF1, using Isonicotinic acid hydrazide instead of Acetic acid hydrazide.

MS (ES⁺): 437 [M+H]⁺.

HPLC (Nucleosil, 6 min method (0-3 min 5-95% ACN, 3.5-5.5 min 95% ACN, 5.5-5.55 min 95-5% ACN, 5.55-6 min 5% ACN): 3.84 min.

### Example DF4

### 7-[cis-4-Aminomethyl-4-(3-chloro-phenyl)-cyclohexyl]-6-oxo-5,6,7,8-tetrahydro-[1,2,4]triazolo[4,3-a]pyrazine-3-carboxylic acid amide dihydrochloride

The title compound was prepared analogously as described in Example DF1, using Oxamic acid hydrazide instead of Acetic acid hydrazide.

MS (ES⁺): 403 [M+H]⁺.

HPLC (Nucleosil, 6 min method (0-3 min 5-95% ACN, 3.5-5.5 min 95% ACN, 5.5-5.55 min 95-5% ACN, 5.55-6 min 5% ACN): 3.86 min.

### Example DF5

### 7-[cis-4-Aminomethyl-4-(3-chloro-phenyl)-cyclohexyl]-3(1H-indol-3-ylmethyl)-7,8-dihydro-[1,2,4]triazolo[4,3-a]pyrazin-6-one dihydrochloride

The title compound was prepared analogously as described in Example DF1, using Indole-3-acetic acid hydrazide instead of Acetic acid hydrazide.

MS (ES⁺): 489 [M+H]⁺.

HPLC (Nucleosil, 6 min method (0-3 min 5-95% ACN, 3.5-5.5 min 95% ACN, 5.5-5.55 min 95-5% ACN, 5.55-6 min 5% ACN): 4.20 min.

### Example DF6

### 7-[trans-4-Aminomethyl-4-(3-chloro-phenyl)-cyclohexyl]-3-(3-methoxy-phenyl)-7,8-dihydro-[1,2,4]triazolo[4,3-a]pyrazin-6-one dihydrochloride

The title compound was prepared analogously as described in Example DF2, using 3-Methoxybenzoic acid hydrazide instead of Acetic acid hydrazide.

MS (ES⁺): 466 [M+H]⁺.

HPLC (Nucleosil, 6 min method (0-3 min 5-95% ACN, 3.5-5.5 min 95% ACN, 5.5-5.55 min 95-5% ACN, 5.55-6 min 5% ACN): 4.05 min.

### Example DF7

### 7-[trans-4-Aminomethyl-4-(3-chloro-phenyl)-cyclohexyl]-3-pyridin-2-yl-7,8-dihydro-[1,2,4]triazolo[4,3-a]pyrazin-6-one dihydrochloride

The title compound was prepared analogously as described in Example DF2, using Pyridine-2-carboxylic acid hydrazide instead of Acetic acid hydrazide.

MS (ES⁺): 437 [M+H]⁺.

HPLC (Nucleosil, 6 min method (0-3 min 5-95% ACN, 3.5-5.5 min 95% ACN, 5.5-5.55 min 95-5% ACN, 5.55-6 min 5% ACN): 3.98 min.

### Example DF8

### 7-[trans-4-Aminomethyl-4-(3-chloro-phenyl)-cyclohexyl]-3-(3,4-dimethoxy-phenyl)-7,8-dihydro-[1,2,4]triazolo[4,3-a]pyrazin-6-one dihydrochloride

The title compound was prepared analogously as described in Example DF2, using 3,4-Dimethoxybenzoic acid hydrazide instead of Acetic acid hydrazide.

MS (ES⁺): 496 [M+H]⁺.

HPLC (Nucleosil, 6 min method (0-3 min 5-95% ACN, 3.5-5.5 min 95% ACN, 5.5-5.55 min 95-5% ACN, 5.55-6 min 5% ACN): 3.98 min.

### Example DF9

### 7-[trans-4-Aminomethyl-4-(3-chloro-phenyl)-cyclohexyl]-3-(1H-indol-3-ylmethyl)-7,8-dihydro-[1,2,4]triazolo[4,3-a]pyrazin-6-one dihydrochloride

The title compound was prepared analogously as described in Example DF2, using Indole-3-acetic acid hydrazide instead of Acetic acid hydrazide.

MS (ES⁺): 489 [M+H]⁺.

HPLC (Nucleosil, 6 min method (0-3 min 5-95% ACN, 3.5-5.5 min 95% ACN, 5.5-5.55 min 95-5% ACN, 5.55-6 min 5% ACN): 4.06 min.

### Example DF10

### 7-[cis-4-Aminomethyl-4-(3-chloro-phenyl)-cyclohexyl]-3-(3-methoxy-phenyl)-7,8-dihydro-[1,2,4]triazolo[4,3-a]pyrazin-6-one dihydrochloride

The title compound was prepared analogously as described in Example DF1, using 3-Methoxybenzoic acid hydrazide instead of Acetic acid hydrazide.

MS (ES⁺): 466 [M+H]⁺.

HPLC (Nucleosil, 6 min method (0-3 min 5-95% ACN, 3.5-5.5 min 95% ACN, 5.5-5.55 min 95-5% ACN, 5.55-6 min 5% ACN): 4.24 min.

### Example DG1

### 3-{7-[trans-4-Aminomethyl-4-(3-chloro-phenyl)-cyclohexyl]-8-oxo-5,6,7,8-tetrahydro-[1,2,4]triazolo[4,3-a]pyrazin-3-yl}-benzoic acid methyl ester dihydrochloride

The title compound was prepared analogously as described in Example D1 step A to H using (2-Amino-ethyl)-carbamic acid benzyl ester hydrochloride instead of 3-trifluoromethyl-5,6,7,8-tetrahydro-[1,2,4]triazolo[4,3-a]pyrazine to afford {2-[4-(tert-Butoxycarbonylamino-methyl)-4-(cis-3-chloro-phenyl)-cyclohexylamino]-ethyl}-carbamic acid benzyl ester and {2-[4-(tert-Butoxycarbonylamino-methyl)-4-(trans-3-chloro-phenyl)-cyclohexylamino]-ethyl}-carbamic acid benzyl ester followed by step

### I) N-(2-Benzyloxycarbonylamino-ethyl)-N-[4-(tert-butoxycarbonylamino-methyl)-4-(trans-3-chloro-phenyl)-cyclohexyl]-oxalamic acid ethyl ester

To a solution of {2-[4-(tert-Butoxycarbonylamino-methyl)-4-(trans-3-chloro-phenyl)-cyclohexylamino]-ethyl}-carbamic acid benzyl ester (451 mg, 0.874 mmol) were added at 0° C. 4-(Dimethylamino)pyridine (11 mg, 0.087 mmol), Triethylamine (608 μl, 4.37 mmol) and Ethyl oxalyl chloride (146 μl, 1.31 mmol). The reaction mixture was stirred at room temperature for 3 days. The mixture was quenched with 1N Hydrochloric acid and the product was extracted 2× into dichloromethane. The combined organic fractions were dried over sodium sulfate and the residue was purified by prep. HPLC (InterChrom C18 ODB 10 μm 28×250 mm, flow 40 ml/min, 45 min method (0-2.5 min 20% ACN, 2.5-42.5 min 20-100% ACN, 42.5-45.0 min 100% ACN). Fractions containing the product were concentrated in vacuo to give the title compound as a white solid.

MS (ES⁺): 616 [M+H]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 4.24 min.

### J) [1-(trans-3-Chloro-phenyl)-4-(2,3-dioxo-piperazin-1-yl)-cyclohexylmethyl]-carbamic acid tert-butyl ester

To a solution of N-(2-Benzyloxycarbonylamino-ethyl)-N-[4-(tert-butoxycarbonylamino-methyl)-4-(trans-3-chloro-phenyl)-cyclohexyl]-oxalamic acid ethyl ester (206 mg, 0.334 mmol) in Ethanol abs. (5 ml) was added Palladium (10% on charcoal) (4 mg, 0.033 mmol) and after purge with nitrogen the reaction mixture was stirred at room temperature under hydrogen atmosphere for 16 h. A further 4 mg of Palladium (10% on charcoal) (0.033 mmol) was added to the reaction mixture under flushed nitrogen atmosphere. Then the reaction mixture was stirred at room temperature under hydrogen atmosphere for 3 h. The black suspension was filtered over Celite and washed with ethanol. The combined filtrates were concentrated in vacuo. The residue was purified by flash chromatography (Silica cartridge) using gradient elution from 100% dichloromethane to dichloromethane:methanol 4:1. Fractions containing the product were concentrated in vacuo to give the title compound as a pale yellow oil.

MS (ES⁺): 438 [M+H]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 3.16 min.

### K) [1-(trans-3-Chloro-phenyl)-4-(5-ethoxy-6-oxo-3,6-dihydro-2H-pyrazin-1-yl)-cyclohexylmethyl]-carbamic acid tert-butyl ester

To a solution of [1-(trans-3-Chloro-phenyl)-4-(2,3-dioxo-piperazin-1-yl)-cyclohexylmethyl]-carbamic acid tert-butyl ester (99 mg, 0.226 mmol) in dichloromethane (8 ml) were added Triethyloxonium tetrafluoroborate (215 mg, 1.13 mmol) and anhydrous sodium carbonate (479 mg, 4.52 mmol). The reaction mixture was stirred at room temperature for 3 h. The mixture was quenched with water and the product was extracted 2× into dichloromethane. The combined organic fractions were dried over sodium sulfate and concentrated in vacuo to give the title compound as a yellow oil.

MS (ES⁺): 464 [M+H]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 3.61 min.

### L) 3-{7-[4-(tert-Butoxycarbonylamino-methyl)-4-(trans-3-chloro-phenyl)-cyclohexyl]-8-oxo-5,6,7,8-tetrahydro-[1,2,4]triazolo[4,3-a]pyrazin-3-yl}-benzoic acid methyl ester

To a solution of [1-(trans-3-Chloro-phenyl)-4-(5-ethoxy-6-oxo-3,6-dihydro-2H-pyrazin-1-yl)-cyclohexylmethyl]-carbamic acid tert-butyl ester (52 mg, 0.113 mmol) in n-Butanol (2 ml) was added 3-Hydrazinocarbonyl-benzoic acid methyl ester (44 mg, 0.23 mmol). The reaction mixture was refluxed for 3 days. The mixture was diluted with dichloromethane and washed with water and brine. The organic layer was dried over sodium sulfate and concentrated in vacuo. The residue was purified by prep. HPLC (Waters SunFire Prep C18 ODB 5 μm 19×50 mm, flow 20 ml/min, 15 min method (0-2.5 min 20% ACN, 2.5-12.5 min 5-100% ACN, 12.5-15.0 min 100% ACN). Fractions containing the product were concentrated in vacuo to give the title compound as a pale yellow oil.

MS (ES⁺): 594, 596 [M+H]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 3.61 min.

### M) 3-{7-[trans-4-Aminomethyl-4-(3-chloro-phenyl)-cyclohexyl]-8-oxo-5,6,7,8-tetrahydro-[1,2,4]triazolo[4,3-a]pyrazin-3-yl}-benzoic acid methyl ester dihydrochloride

Trifluoroacetic acid (0.2 mL) was added to a solution of 3-{7-[4-(tert-Butoxycarbonylamino-methyl)-4-(trans-3-chloro-phenyl)-cyclohexyl]-8-oxo-5,6,7,8-tetrahydro-[1,2,4]triazolo[4,3-a]pyrazin-3-yl}-benzoic acid methyl ester (7 mg, 0.011 mmol) in dichloromethane (1 mL). The reaction mixture was stirred at room temperature for 1 h. The mixture was concentrated in vacuo. The residue was purified by prep. HPLC (Nucleosil C18 HD 5 μm 21×50 mm, flow 20 ml/min, 15 min method (0-2.5 min 5% ACN, 2.5-12.5 min 5-100% ACN, 12.5-15.0 min 100% ACN). Fractions containing the product were concentrated in vacuo to give the formate salt of the title compound, which was dissolved in methanol and treated with an excess of 2M hydrogen chloride in methanol. Removal of the volatiles gave the title compound as a white solid.

MS (ES⁺): 494 [M+H]⁺.

HPLC (Nucleosil, 6 min method (0-3 min 5-95% ACN, 3.5-5.5 min 95% ACN, 5.5-5.55 min 95-5% ACN, 5.55-6 min 5% ACN): 4.22 min.

### Example DG2

### 7-[trans-4-Aminomethyl-4-(3-chloro-phenyl)-cyclohexyl]-3-pyridin-3-yl-6,7-dihydro-5H-[1,2,4]triazolo[4,3-a]pyrazin-8-one dihydrochloride

The title compound was prepared analogously as described in Example DG1, using Nicotinic acid hydrazide instead of 3-Hydrazinocarbonyl-benzoic acid methyl ester.

MS (ES⁺): 437 [M+H]⁺.

HPLC (Nucleosil, 6 min method (0-3 min 5-95% ACN, 3.5-5.5 min 95% ACN, 5.5-5.55 min 95-5% ACN, 5.55-6 min 5% ACN): 3.79 min.

### Example DG3

### 7-[trans-4-Aminomethyl-4-(3-chloro-phenyl)-cyclohexyl]-3-(1H-indol-2-ylmethyl)-6,7-dihydro-5H-[1,2,4]triazolo[4,3-a]pyrazin-8-one dihydrochloride

The title compound was prepared analogously as described in Example DG1, using Indole-3-acetic acid hydrazide instead of 3-Hydrazinocarbonyl-benzoic acid methyl ester.

MS (ES⁺): 487, 489 [M+H]⁺.

HPLC (Waters Symmetry C18 3.5 μm 2.1×50 mm, 6 min method (0-3 min 5-95% ACK 3.5-5.5 min 95% ACN, 5.5-5.55 min 95-5% ACN, 5.55-6 min 5% ACN): 3.03 min.

### Example DG4

### 7-[trans-4-Aminomethyl-4-(3-chloro-phenyl)-cyclohexyl]-3-[3-(4-methoxy-phenyl)-isoxazol-5-yl]-6,7-dihydro-5H-[1,2,4]triazolo[4,3-a]pyrazin-8-one dihydrochloride

The title compound was prepared analogously as described in Example DG1, using 3-(4-Methoxy-phenyl)-isoxazole-5-carboxylic acid hydrazide instead of 3-Hydrazinocarbonyl-benzoic acid methyl ester.

MS (ES⁺): 533 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.29 min.

### Example DG5

### 7-[cis-4-Aminomethyl-4-(3-chloro-phenyl)-cyclohexyl]-3-(1H-indol-2-ylmethyl)-6,7-dihydro-5H-[1,2,4]triazolo[4,3-a]pyrazin-8-one dihydrochloride

The title compound was prepared analogously as described in Example DG1, using Indole-3-acetic acid hydrazide instead of 3-Hydrazinocarbonyl-benzoic acid methyl ester, step I from {2-[4-(tert-Butoxycarbonylamino-methyl)-4-(cis-3-chloro-phenyl)-cyclohexylamino]-ethyl}-carbamic acid benzyl ester.

MS (ES⁺): 488, 489 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.00 min.

### Example DG6

### 7-[cis-4-Aminomethyl-4-(3-chloro-phenyl)-cyclohexyl]-3-cyclopropyl-6,7-dihydro-5H-[1,2,4]triazolo[4,3-a]pyrazin-8-one dihydrochloride

The title compound was prepared analogously as described in Example DG5, using Cyclopropane carboxylic acid hydrazide instead of Indole-3-acetic acid hydrazide.

MS (ES⁺): 400 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.73 min.

### Example DG7

### 7-[cis-4-Aminomethyl-4-(3-chloro-phenyl)-cyclohexyl]-3-(3,4-dimethoxy-phenyl)-6,7-dihydro-5H-[1,2,4]triazolo[4,3-a]pyrazin-8-one dihydrochloride

The title compound was prepared analogously as described in Example DG5, using 3,4-Dimethoxybenzoic acid hydrazide instead of Indole-3-acetic acid hydrazide.

MS (ES⁺): 496 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.91 min.

### Example DG8

### 7-[cis-4-Aminomethyl-4-(3-chloro-phenyl)-cyclohexyl]-3-(4-methoxy-phenyl)-6,7-dihydro-5H-[1,2,4]triazolo[4,3-a]pyrazin-8-one dihydrochloride

The title compound was prepared analogously as described in Example DG5, using 4-Methoxybenzoic acid hydrazide instead of Indole-3-acetic acid hydrazide.

MS (ES⁺): 466 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.96 min.

### Example DG9

### 7-[cis-4-Aminomethyl-4-(3-chloro-phenyl)-cyclohexyl]-3-(3-methanesulfonyl-phenyl)-6,7-dihydro-5H-[1,2,4]triazolo[4,3-a]pyrazin-8-one dihydrochloride

The title compound was prepared analogously as described in Example DG5, using 3-Methanesulfonyl-benzoic acid hydrazide instead of Indole-3-acetic acid hydrazide.

MS (ES⁺): 514 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.85 min.

### Example DG10

### 7-[cis-4-Aminomethyl-4-(3-chloro-phenyl)-cyclohexyl]-3-(4-fluoro-phenyl)-6,7-dihydro-5H-[1,2,4]triazolo[4,3-a]pyrazin-8-one dihydrochloride

The title compound was prepared analogously as described in Example DG5, using 4-Fluorobenzoic acid hydrazide instead of Indole-3-acetic acid hydrazide.

MS (ES⁺): 454 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.10 min.

### Example DG11

### 7-[cis-4-Aminomethyl-4-(3-chloro-phenyl)-cyclohexyl]-3-(3-fluoro-phenyl)-6,7-dihydro-5H-[1,2,4]triazolo[4,3-a]pyrazin-8-one dihydrochloride

The title compound was prepared analogously as described in Example DG5, using 3-Fluorobenzoic acid hydrazide instead of Indole-3-acetic acid hydrazide.

MS (ES⁺): 454 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.05 min.

### Example DG12

### 7-[cis-4-Aminomethyl-4-(3-chloro-phenyl)-cyclohexyl]-8-oxo-5,6,7,8-tetrahydro-[1,2,4]triazolo[4,3-a]pyrazine-3-carboxylic acid amide dihydrochloride

The title compound was prepared analogously as described in Example DG5, using Oxamic acid hydrazide instead of Indole-3-acetic acid hydrazide.

MS (ES⁺): 403 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.86 min.

### Example DG13

### 7-[cis-4-Aminomethyl-4-(3-chloro-phenyl)-cyclohexyl]-3-(4H-[1,2,4]triazol-3-yl)-6,7-dihydro-5H-[1,2,4]triazolo[4,3-a]pyrazin-8-one trihydrochloride

The title compound was prepared analogously as described in Example DG5, using 1H-[1,2,4]triazole-3-carboxylic acid hydrazide instead of Indole-3-acetic acid hydrazide.

MS (ES⁺): 427 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.85 min.

### Example DG14

### 7-[cis-4-Aminomethyl-4-(3-chloro-phenyl)-cyclohexyl]-3-pyridin-4-yl-6,7-dihydro-5H-[1,2,4]triazolo[4,3-a]pyrazin-8-one dihydrochloride

The title compound was prepared analogously as described in Example DG5, using Isonicotinic acid hydrazide instead of Indole-3-acetic acid hydrazide.

MS (ES⁺): 437 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.79 min.

### Example DG15

### 7-[cis-4-Aminomethyl-4-(3-chloro-phenyl)-cyclohexyl]-3-methyl-6,7-dihydro-5H-[1,2,4]triazolo[4,3-a]pyrazin-8-one dihydrochloride

The title compound was prepared analogously as described in Example DG5, using Acetic acid hydrazide instead of Indole-3-acetic acid hydrazide.

MS (ES⁺): 374 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.79 min.

### Example DG16

### 3-{7-[cis-4-Aminomethyl-4-(3-chloro-phenyl)-cyclohexyl]-8-oxo-5,6,7,8-tetrahydro-[1,2,4]triazolo[4,3-a]pyrazin-3-yl}-benzoic acid dihydrochloride

The title compound was prepared analogously as described in Example DG5, using 3-Hydrazinocarbonyl benzoic acid instead of Indole-3-acetic acid hydrazide.

MS (ES⁺): 480 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.11 min.

### Example DG17

### 7-[cis-4-Aminomethyl-4-(3-chloro-phenyl)-cyclohexyl]-3-[3-(4-fluoro-phenyl)-isoxazol-5-yl]-6,7-dihydro-5H-[1,2,4]triazolo[4,3-a]pyrazin-8-one dihydrochloride

The title compound was prepared analogously as described in Example DG5, using 3-(4-Fluoro-phenyl)-isoxazole-5-carboxylic acid hydrazide dihydrochloride instead of Indole-3-acetic acid hydrazide.

MS (ES⁺): 521 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.32 min.

### Example DG18

### 7-[cis-4-Aminomethyl-4-(3-chloro-phenyl)-cyclohexyl]-3-(2-hydroxy-propyl)-6,7-dihydro-5H-[1,2,4]triazolo[4,3-a]pyrazin-8-one dihydrochloride

The title compound was prepared analogously as described in Example DG5, using 3-Hydroxybutanohydrazide instead of Indole-3-acetic acid hydrazide.

MS (ES⁺): 418 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.71 min.

### Example DG19

### 7-[cis-4-Aminomethyl-4-(3-chloro-phenyl)-cyclohexyl]-3-(2-methoxy-ethyl)-6,7-dihydro-5H-[1,2,4]triazolo[4,3-a]pyrazin-8-one dihydrochloride

The title compound was prepared analogously as described in Example DG5, using 3-Methoxypropionic acid hydrazide instead of Indole-3-acetic acid hydrazide.

MS (ES⁺): 418 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.79 min.

### Example DG20

### 4-{7-[cis-4-Aminomethyl-4-(3-chloro-phenyl)-cyclohexyl]-8-oxo-5,6,7,8-tetrahydro-[1,2,4]triazolo[4,3-a]pyrazin-3-yl}-2-methyl-2H-phthalazin-1-one dihydrochloride

The title compound was prepared analogously as described in Example DG5, using 3-Methyl-4-oxo-3,4-dihydro-phthalazine-1-carboxylic acid hydrazide instead of Indole-3-acetic acid hydrazide.

MS (ES⁺): 518 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.03 min.

### Example DG21

### 4-{7-[cis-4-Aminomethyl-4-(3-chloro-phenyl)-cyclohexyl]-8-oxo-5,6,7,8-tetrahydro-[1,2,4]triazolo[4,3-a]pyrazin-3-yl}-benzamide dihydrochloride

The title compound was prepared analogously as described in Example DG5, using 4-(Hydrazinocarbonyl)benzamide instead of Indole-3-acetic acid hydrazide. The product of step L [4-[3-(4-Carbamoyl-phenyl)-8-oxo-5,6-dihydro-8H-[1,2,4]triazolo[4,3-a]pyrazin-7-yl]-1-(3-chloro-phenyl)-cyclohexylmethyl]-carbamic acid tert-butyl ester was partly esterified during boc deprotection step M when it was treated with 2N HCl in methanol. The resulting two compounds 4-{7-[cis-4-Aminomethyl-4-(3-chloro-phenyl)-cyclohexyl]-8-oxo-5,6,7,8-tetrahydro-[1,2,4]triazolo[4,3-a]pyrazin-3-yl}-benzamide and 4-{7-[cis-4-Aminomethyl-4-(3-chloro-phenyl)-cyclohexyl]-8-oxo-5,6,7,8-tetrahydro-[1,2,4]triazolo[4,3-a]pyrazin-3-yl}-benzoic acid methyl ester were separated by prep. HPLC. See also example DG26.

MS (ES⁺): 518 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.03 min.

### Example DG22

### 7-[cis-4-Aminomethyl-4-(3-chloro-phenyl)-cyclohexyl]-8-oxo-5,6,7,8-tetrahydro-[1,2,4]triazolo[4,3-a]pyrazine-3-carboxylic acid isopropylamide dihydrochloride

The title compound was prepared analogously as described in Example DG5, using 2-Hydrazino-N-isopropyl-2-oxoacetamide instead of Indole-3-acetic acid hydrazide.

MS (ES⁺): 518 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.03 min.

### Example DG23

### 3-(2-{7-[cis-4-Aminomethyl-4-(3-chloro-phenyl)-cyclohexyl]-8-oxo-5,6,7,8-tetrahydro-[1,2,4]triazolo[4,3-a]pyrazin-3-yl}-ethyl)-5,5-dimethyl-imidazolidine-2,4-dione trihydrochloride

The title compound was prepared analogously as described in Example DG5, using 3-(4,4-Dimethyl-2,5-dioxo-imidazolidin-1-yl) propionic acid hydrazide instead of Indole-3-acetic acid hydrazide.

MS (ES⁺): 518 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.03 min.

### Example DG24

### 2-{7-[cis-4-Aminomethyl-4-(3-chloro-phenyl)-cyclohexyl]-8-oxo-5,6,7,8-tetrahydro-[1,2,4]triazolo[4,3-a]pyrazin-3-yl}-N-methyl-acetamide dihydrochloride

The title compound was prepared analogously as described in Example DG5, using 3-Hydrazino-N-methyl-3-oxopropanamide instead of Indole-3-acetic acid hydrazide.

MS (ES⁺): 518 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.03 min.

### Example DG25

### 7-[cis-4-Aminomethyl-4-(3-chloro-phenyl)-cyclohexyl]-8-oxo-5,6,7,8-tetrahydro-[1,2,4]triazolo[4,3-a]pyrazine-3-carboxylic acid cyclopropylamide dihydrochloride

The title compound was prepared analogously as described in Example DG5, using N-Cyclopropyl-2-hydrazino-2-oxoacetamide instead of Indole-3-acetic acid hydrazide.

MS (ES⁺): 518 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.03 min.

### Example DG26

### 4-{7-[cis-4-Aminomethyl-4-(3-chloro-phenyl)-cyclohexyl]-8-oxo-5,6,7,8-tetrahydro-[1,2,4]triazolo[4,3-a]pyrazin-3-yl}-benzoic acid methyl ester dihydrochloride

The title compound was prepared analogously as described in Example DG21. The product of step L [4-[3-(4-Carbamoyl-phenyl)-8-oxo-5,6-dihydro-8H-[1,2,4]triazolo[4,3-a]pyrazin-7-yl]-1-(3-chloro-phenyl)-cyclohexylmethyl]-carbamic acid tert-butyl ester was partly esterified during boc deprotection step M when it was treated with 2N HCl in methanol. The resulting two compounds 4-{7-[cis-4-Aminomethyl-4-(3-chloro-phenyl)-cyclohexyl]-8-oxo-5,6,7,8-tetrahydro-[1,2,4]triazolo[4,3-a]pyrazin-3-yl}-benzamide and 4-{7-(cis-4-Aminomethyl-4-(3-chloro-phenyl)-cyclohexyl]-8-oxo-5,6,7,8-tetrahydro-[1,2,4]triazolo[4,3-a]pyrazin-3-yl}-benzoic acid methyl ester were separated by prep. HPLC. See also example DG21.

MS (ES⁺): 518 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.03 min

### Example DG27

### 2-{7-[cis-4-Aminomethyl-4-(3-chloro-phenyl)-cyclohexyl]-8-oxo-5,6,7,8-tetrahydro-[1,2,4]triazolo[4,3-a]pyrazin-3-yl}-acetamide trihydrochloride

The title compound was prepared analogously as described in Example DG5, using 3-Hydrazino-3-oxo propanamide instead of Indole-3-acetic acid hydrazide.

MS (ES⁺): 417 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.69 min.

### Example DG28

### 7-[cis-4-Aminomethyl-4-(3-chloro-phenyl)-cyclohexyl]-3-cyclobutyl-6,7-dihydro-5H-[1,2,4]triazolo[4,3-a]pyrazin-8-one dihydrochloride

The title compound was prepared analogously as described in Example DG5, using Cyclobutanecarboxylic acid hydrazide dihydrochloride instead of Indole-3-acetic acid hydrazide.

MS (ES⁺): 414 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.83 min.

### Example DG29

### 7-[cis-4-Aminomethyl-4-(3-chloro-phenyl)-cyclohexyl]-3-(2-methoxy-pyrimidin-5-yl)-6,7-dihydro-5H-[1,2,4]triazolo[4,3-a]pyrazin-8-one dihydrochloride

The title compound was prepared analogously as described in Example DG5, using 2-Methoxy-pyrimidine-5-carboxylic acid hydrazide dihydrochloride instead of Indole-3-acetic acid hydrazide.

MS (ES⁺): 468 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.79 min.

### Example DG30

### 7-[cis-4-Aminomethyl-4-(3-chloro-phenyl)-cyclohexyl]-3-(3-fluoro-pyridin-4-yl)-6,7-dihydro-5H-[1,2,4]triazolo[4,3-a]pyrazin-8-one dihydrochloride

The title compound was prepared analogously as described in Example DG5, using 3-Fluoro-isonicotinic acid hydrazide dihydrochloride instead of Indole-3-acetic acid hydrazide.

MS (ES⁺): 455 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.79 min.

### Example DG31

### 3-{7-[cis-4-Aminomethyl-4-(3-chloro-phenyl)-cyclohexyl]-8-oxo-5,6,7,8-tetrahydro-[1,2,4]triazolo[4,3-a]pyrazin-3-yl}-N-methyl-benzamide dihydrochloride

The title compound was prepared analogously as described in Example DG5, step A to L using 3-Hydrazinocarbonyl-benzoic acid dihydrochloride instead of Indole-3-acetic acid hydrazide to afford 3-{7-[4-(tert-Butoxycarbonylamino-methyl)-4-(cis-3-chloro-phenyl)-cyclohexyl]-8-oxo-5,6,7,8-tetrahydro-[1,2,4]triazolo[4,3-a]pyrazin-3-yl}-benzoic acid followed by step:

### M) {1-(cis-3-Chloro-phenyl)-4-[3-(3-methylcarbamoyl-phenyl)-8-oxo-5,6-dihydro-8H-[1,2,4]triazolo[4,3-a]pyrazin-7-yl]-cyclohexylmethyl}-carbamic acid tert-butyl ester

To a solution of 3-{7-[4-(tert-Butoxycarbonylamino-methyl)-4-(cis-3-chloro-phenyl)-cyclohexyl]-8-oxo-5,6,7,8-tetrahydro-[1,2,4]triazolo[4,3-a]pyrazin-3-yl}-benzoic acid (33 mg, 0.057 mmol) in dichloromethane (2 ml) was added 0-(Benzotriazol-1-yl)-N,N,N′,N′-tetramethyluronium hexafluorophosphate (43 mg, 0.114 mmol), Diisopropylethylamine (20 μl, 0.114 mmol) and Methylamine hydrochloride (6 mg, 0.086 mmol). The reaction mixture was stirred at room temperature for 16 h. The mixture was diluted with dichloromethane and washed with water, 1N Hydrochloric acid, saturated aqueous sodium bicarbonate solution and brine. The organic layer was dried over sodium sulfate and concentrated in vacuo. The residue was purified over silica gel cartridge by MPLC (ISCO Companion) eluting with dichloromethane to dichloromethane/methanol 9:1. Fractions containing the product were concentrated in vacuo to give the title compound as a yellow solid.

MS (ES⁺): 593 [M+H]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 3.47 min.

### N) 3-{7-[cis-4-Aminomethyl-4-(3-chloro-phenyl)-cyclohexyl]-8-oxo-5,6,7,8-tetrahydro-[1,2,4]triazolo[4,3-a]pyrazin-3-yl}-N-methyl-benzamide dihydrochloride

Trifluoroacetic acid (0.2 mL) was added to a solution of {1-(cis-3-Chloro-phenyl)-4-[3-(3-methylcarbamoyl-phenyl)-8-oxo-5,6-dihydro-8H-[1,2,4]triazolo[4,3-a]pyrazin-7-yl]-cyclohexylmethyl}-carbamic acid tert-butyl ester (15 mg, 0.025 mmol) in dichloromethane (0.5 mL). The reaction mixture was stirred at room temperature for 2 h. The mixture was concentrated in vacuo. The residue was purified by prep. HPLC (Nucleosil C18 HD 5 μm 21×50 mm, flow 20 ml/min, 15 min method (0-2.5 min 5% ACN, 2.5-12.5 min 5-100% ACN, 12.5-15.0 min 100% ACN). Fractions containing the product were concentrated in vacuo to give the formate salt of the title compound, which was dissolved in 2M hydrogen chloride in methanol. Methanol was removed by evaporation. The residue was dissolved in dioxane, frozen and lyophilized to give the title compound as a white solid.

MS (ES⁺): 493 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.81 min.

### Example DG32

### 7-[cis-4-Aminomethyl-4-(3-chloro-phenyl)-cyclohexyl]-3-(3-fluoro-pyridin-4-yl)-6,7-dihydro-5H-[1,2,4]triazolo[4,3-a]pyrazin-8-one dihydrochloride

The title compound was prepared analogously as described in Example DG31, using Morpholine instead of Methylamine hydrochloride.

MS (ES⁺): 549 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.87 min.

### Example DH1

### N-[cis-4-Aminomethyl-4-(3-chloro-phenyl)-cyclohexyl]-N-[2-(3-methanesulfonyl-phenyl)-ethyl]-acetamide hydrochloride

The title compound was prepared analogously as described in Example D1 step A to H, using 2-(3-Methanesulfonyl-phenyl)-ethylamine instead of 3-trifluoromethyl-5,6,7,8-tetrahydro-[1,2,4]triazolo[4,3-a]pyrazine to afford {1-(cis-3-Chloro-phenyl)-4-[2-(3-methanesulfonyl-phenyl)-ethylamino]-cyclohexylmethyl}-carbamic acid tert-butyl ester and {1-(cis-3-Chloro-phenyl)-4-[2-(3-methanesulfonyl-phenyl)-ethylamino]-cyclohexylmethyl}-carbamic acid tert-butyl ester followed by step

### I) [4-{Acetyl-[2-(3-methanesulfonyl-phenyl)-ethyl]-amino}-1-(cis-3-chloro-phenyl)-cyclohexylmethyl]-carbamic acid tert-butyl ester

To a mixture of {1-(cis-3-Chloro-phenyl)-4-[2-(3-methanesulfonyl-phenyl)-ethylamino]-cyclohexylmethyl}-carbamic acid tert-butyl ester (30 mg, 0.058 mmol) and Diisopropylethylamine (22 μL, 0.127 mmol) in dichloromethane (1 ml) was added a solution of Acetylchloride (5 μl, 0.069 mmol) in dichloromethane (1 ml) dropwise at room temperature. The resulting mixture was stirred at room temperature for 5 minutes. The mixture was concentrated in vacuo. The residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were lyophilized in vacuo to give the title compound as a white solid.

MS (ES⁺): 508 [M-tBu+H]⁺.

### J) N-[cis-4-Aminomethyl-4-(3-chloro-phenyl)-cyclohexyl]-N-[2-(3-methanesulfonyl-phenyl)-ethyl]-acetamide hydrochloride

To [4-{Acetyl-[2-(3-methanesulfonyl-phenyl)-ethyl]-amino}-1-(cis-3-chloro-phenyl)-cyclohexylmethyl]-carbamic acid tert-butyl ester (23 mg, 0.041 mmol) was added 4N hydrogen chloride solution in dioxane (5 ml). The reaction mixture stirred at room temperature for 1 h, then it was concentrated in vacuo. The residue was treated with diethyl ether in ultrasonic bath. The etheric phase was removed with a pipette. The residue was lyophilized in vacuo to give the title compound as a white solid.

MS (ES⁺): 463 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 4.08 min.

### Example DH2

### N-[cis-4-Aminomethyl-4-(3-chloro-phenyl)-cyclohexyl]-N-(4-methanesulfonyl-benzyl)-acetamide hydrochloride

The title compound was prepared analogously as described in Example DH1, using 4-Methanesulfonyl benzylamide hydrochloride instead of 2-(3-Methanesulfonyl-phenyl)-ethylamine.

MS (ES⁺): 449 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.80 min.

### Example DH3

### N-[cis-4-Aminomethyl-4-(3-chloro-phenyl)-cyclohexyl]-N-[2-(3-methanesulfonylamino-phenyl)-ethyl]-acetamide

The title compound was prepared analogously as described in Example DH1, using N-[3-(2-Amino-ethyl)-phenyl]-methanesulfonamide instead of 2-(3-Methanesulfonyl-phenyl)-ethylamine.

MS (ES⁺): 478 [M+H]⁺.

### Example DH4

### Cyclopropanecarboxylic acid [cis-4-aminomethyl-4-(3-chloro-phenyl)-cyclohexyl]-[2-(3-methanesulfonyl-phenyl)-ethyl]-amide hydrochloride

The title compound was prepared analogously as described in Example DH1, using Cyclopropanecarbonyl chloride instead of Acetyl chloride.

MS (ES⁺): 489 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 1.61 min.

### Example DH5

### N-[cis-4-Aminomethyl-4-(3-chloro-phenyl)-cyclohexyl]-N-[2-(3-methanesulfonyl-phenyl)-ethyl]-propionamide hydrochloride

The title compound was prepared analogously as described in Example DH1, using Propionyl chloride instead of Acetyl chloride.

MS (ES⁺): 477 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 1.11 min.

### Example DH6

### N-[cis-4-Aminomethyl-4-(3-chloro-phenyl)-cyclohexyl]-N-[2-(3-methanesulfonyl-phenyl)-ethyl]-methanesulfonamide hydrochloride

The title compound was prepared analogously as described in Example DH1, using Methanesulfonyl chloride instead of Acetyl chloride.

MS (ES⁺): 499 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 2.38 min.

### Example DH7

### [cis-4-Aminomethyl-4-(3-chloro-phenyl)-cyclohexyl]-[2-(3-methanesulfonyl-phenyl]-ethyl]-carbamic acid methyl ester hydrochloride

The title compound was prepared analogously as described in Example DH1, using Methyl chloroformate instead of Acetyl chloride.

MS (ES⁺): 479 [M+H]⁺.

### Example DH8

### [cis-4-Aminomethyl-4-(3-chloro-phenyl)-cyclohexyl]-[2-(3-methanesulfonyl-phenyl)-ethyl]-carbamic acid methyl ester hydrochloride

The title compound was prepared analogously as described in Example DH1, using 4-Morpholinecarbonylchloride instead of Acetyl chloride.

MS (ES⁺): 534 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.42 min.

### Example DH9

### 1-[cis-4-Aminomethyl-4-(3-chloro-phenyl)-cyclohexyl]-1-[2-(3-methanesulfonyl-phenyl)-ethyl]-3-methyl-urea hydrochloride

The title compound was prepared according to Scheme D.

The title compound was prepared analogously as described in Example DH1, using Methyl isocyanate instead of Acetyl chloride and Triethylamine instead of Diisopropylethylamine.

MS (ES⁺): 478 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.30 min.

### Example DI1

### 3-{3-[cis-4-Aminomethyl-4-(3-chloro-phenyl)-cyclohexyl]-2-oxo-imidazolidin-1-yl}-benzoic acid methyl ester hydrochloride

The title compound was prepared analogously as described in Example D1 step A to H using (2-Amino-ethyl)-carbamic acid benzyl ester hydrochloride instead of 3-trifluoromethyl-5,6,7,8-tetrahydro-[1,2,4]triazolo[4,3-a]pyrazine to afford {2-[4-(tert-Butoxycarbonylamino-methyl)-4-(cis-3-chloro-phenyl)-cyclohexylamino)-ethyl}-carbamic acid benzyl ester and {2-[4-(tert-Butoxycarbonylamino-methyl)-4-(trans-3-chloro-phenyl)-cyclohexylamino]-ethyl}-carbamic acid benzyl ester followed by step

### I) [1-(cis-3-Chloro-phenyl)-4-(2-oxo-imidazolidin-1-yl)-cyclohexylmethyl]-carbamic acid tert-butyl ester

To a solution of {2-[4-(tert-Butoxycarbonylamino-methyl)-4-(cis-3-chloro-phenyl)-cyclohexylamino]-ethyl}-carbamic acid benzyl ester (720 mg, 1.40 mmol) in Dimethylformamide (15 ml) was added Cesiumcarbonate (2.28 g, 7.00 mmol). The mixture was stirred for 3 h at 90° C. The reaction mixture was treated with aqueous Sodium bicarbonate solution (conc.) The product was extracted 2× into dichloromethane. The combined organic extracts were dried over magnesium sulfate. The filtrate was concentrated in vacuo to afford a mixture of the title compound and 1-[cis-4-Aminomethyl-4-(3-chloro-benzyl)-cyclohexyl]-imidazolidin-2-one, which was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were lyophilized in vacuo to give the title compound as a white solid.

MS (ES⁺): 408 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 4.56 min.

### J) 3-{3-[4-(tert-Butoxycarbonylamino-methyl)-4-(cis-3-chloro-phenyl)-cyclohexyl]-2-oxo-imidazolidin-1-yl}-benzoic acid methyl ester

To a solution of [1-(cis-3-Chloro-phenyl)-4-(2-oxo-imidazolidin-1-yl)-cyclohexylmethyl]-carbamic acid tert-butyl ester (50 mg, 0.123 mmol) in toluene (1 ml) was added 3-Bromo-benzoic acid methyl ester (26 mg, 0.123 mmol), Cesiumcarbonate (56 mg, 0.172 mmol), (±)-2,2′-Bis(diphenylphosphino)-1,1′-binaphthalene (6 mg, 0.01 mmol) and Tris(dibenzylideneacetone)dipalladium(0) (5 mg, 0.005 mmol). The mixture was stirred for 2.5 h at 100° C. The reaction mixture was filtered, then the filtrate was concentrated in vacuo to give the title compound as a white solid.

MS (ES⁺): 559 [M+H2O]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 6.31 min.

### K) 3-{3-[cis-4-Aminomethyl-4-(3-chloro-phenyl)-cyclohexyl]-2-oxo-imidazolidin-1-yl}-benzoic acid methyl ester hydrochloride

To 3-{3-[4-(tert-Butoxycarbonylamino-methyl)-4-(cis-3-chloro-phenyl)-cyclohexyl]-2-oxo-imidazolidin-1-yl}-benzoic acid methyl ester (66 mg, 0.122 mmol) was added 4N hydrogen chloride solution in dioxane (3 ml). The reaction mixture stirred at room temperature for 2 h, then it was concentrated in vacuo. The residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were lyophilized in vacuo to give the formate salt of the title compound, which was dissolved in methanol and treated with an excess of 2M hydrogen chloride in methanol. Removal of the volatiles gave the title compound as a white solid.

MS (ES⁺): 442 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 3.44 min.

### Example DI2

### 4-{3-[cis-4-Aminomethyl-4-(3-chloro-phenyl)-cyclohexyl]-2-oxo-imidazolidin-1-yl}-benzoic acid methyl ester hydrochloride

The title compound was prepared analogously as described in Example DI1, using 4-Bromo-benzoic acid methyl ester instead of 3-Bromo-benzoic acid methyl ester.

MS (ES⁺): 442 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 3.53 min.

### Example DI3

### 1-[cis-4-Aminomethyl-4-(3-chloro-phenyl)-cyclohexyl]-3-(4-methanesulfonyl-phenyl)-imidazolidin-2-one hydrochloride

The title compound was prepared analogously as described in Example DI1, using 1-Bromo-4-methanesulfonyl-benzene instead of 3-Bromo-benzoic acid methyl ester.

MS (ES⁺): 462 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 3.05 min.

### Example DI4

### 1-[cis-4-Aminomethyl-4-(3-chloro-phenyl)-cyclohexyl]-3-(3-methanesulfonyl-phenyl)-imidazolidin-2-one hydrochloride

The title compound was prepared analogously as described in Example DI1, using 1-Bromo-3-methanesulfonyl-benzene instead of 3-Bromo-benzoic acid methyl ester.

MS (ES⁺): 462 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 3.10 min.

### Example DI5

### 3-{3-[4-Aminomethyl-4-(3-chloro-phenyl)-cyclohexyl]-2-oxo-imidazolid in-1-yl}-benzoic acid hydrochloride

The title compound was prepared analogously as described in Example D1 step A to K to afford 3-{3-[cis-4-Aminomethyl-4-(3-chloro-phenyl)-cyclohexyl]-2-oxo-imidazolidin-1-yl}-benzoic acid methyl ester hydrochloride followed by step

### L) 3-{3-[4-Aminomethyl-4-(3-chloro-phenyl)-cyclohexyl]-2-oxo-imidazolidin-1-yl}-benzoic acid hydrochloride

To a solution of 3-{3-[cis-4-Aminomethyl-4-(3-chloro-phenyl)-cyclohexyl]-2-oxo-imidazolidin-1-yl}-benzoic acid methyl ester hydrochloride (15 mg, 0.034 mmol) in dioxane (1 ml) was added 1N aqueous Potassium hydroxide solution (0.5 ml). The reaction mixture was treated with microwave at 120° C. for 5 min, then it was directly purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were lyophilized in vacuo to give the formate salt of the title compound, which was dissolved in methanol and treated with an excess of 2M hydrogen chloride in methanol. Removal of the volatiles gave the title compound as a white solid.

MS (ES⁺): 428 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 3.03 min.

### Example DI6

### 4-{3-[4-Aminomethyl-4-(3-chloro-phenyl)-cyclohexyl]-2-oxo-imidazolidin-1-yl}-benzoic acid hydrochloride

The title compound was prepared analogously as described in Example DI5, using 4-Bromo-benzoic acid methyl ester instead of 3-Bromo-benzoic acid methyl ester.

MS (ES⁺): 442 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 2.93 min.

### Example DI7

### 3-{3-[cis-4-Aminomethyl-4-(3-chloro-phenyl)-cyclohexyl]-2-oxo-imidazolidin-1-yl}-benzenesulfonamide hydrochloride

The title compound was prepared analogously as described in Example DI1, using N-(tert-butoxycarbonyl)-(3-bromophenyl)-sulfonamide instead of 3-Bromo-benzoic acid methyl ester.

MS (ES⁺): 463 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 2.82 min.

### Example DI8

### 4-{3-[cis-4-Aminomethyl-4-(3-chloro-phenyl)-cyclohexyl]-2-oxo-imidazolidin-1-yl}-benzenesulfonamide hydrochloride

The title compound was prepared analogously as described in Example DI1, using N-(tert-butoxycarbonyl)-(4-bromophenyl)-sulfonamide instead of 3-Bromo-benzoic acid methyl ester.

MS (ES⁺): 463 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 2.77 min.

### Example 019

### 1-[cis-4-Aminomethyl-4-(3-chloro-phenyl)-cyclohexyl]-3-(3-amino-phenyl)-imidazolidin-2-one hydrochloride

The title compound was prepared analogously as described in Example DI1, using (3-Bromo-phenyl)-carbamic acid tert-butyl ester instead of 3-Bromo-benzoic acid methyl ester.

MS (ES⁺): 399 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 2.05 min.

### Example DI10

### 5-{3-[cis-4-Aminomethyl-4-(3-chloro-phenyl)-cyclohexyl]-2-oxo-imidazolidin-1-yl}-nicotinic acid methyl ester hydrochloride

The title compound was prepared analogously as described in Example DI1, using 5-Bromo-nicotinic acid methyl ester instead of 3-Bromo-benzoic acid methyl ester.

MS (ES⁺): 443 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 2.68 min.

### Example DI11

### 5-{3-[cis-4-Aminomethyl-4-(3-chloro-phenyl)-cyclohexyl]-2-oxo-imidazolidin-1-yl}-nicotinic acid hydrochloride

The title compound was prepared analogously as described in Example DI5, using 5-Bromo-nicotinic acid methyl ester instead of 3-Bromo-benzoic acid methyl ester.

MS (ES⁺): 429 [M+H].

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 1.95 min.

### Example DI12

### 5-{3-[cis-4-Aminomethyl-4-(3-chloro-phenyl)-cyclohexyl]-2-oxo-imidazolidin-1-yl}-thiophene-2-carboxylic acid methyl ester hydrochloride

The title compound was prepared analogously as described in Example DI1, using 5-Bromo-thiophene-2-carboxylic acid methyl ester instead of 3-Bromo-benzoic acid methyl ester.

MS (ES⁺): 448 [Mi-H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 3.38 min.

### Example DI13

### 1-[cis-4-Aminomethyl-4-(3-chloro-phenyl)-cyclohexyl]-3-pyrimidin-5-yl-imidazolidin-2-one hydrochloride

The title compound was prepared analogously as described in Example DI1, using 5-Bromo-pyrimidine instead of 3-Bromo-benzoic acid methyl ester.

MS (ES⁺): 386 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 2.40 min.

### Example DI14

### 5-{3-[cis-4-Aminomethyl-4-(3-chloro-phenyl)-cyclohexyl]-2-oxo-imidazolidin-1-yl}-thiophene-2-carboxylic acid hydrochloride

The title compound was prepared analogously as described in Example DI5, using 5-Bromo-thiophene-2-carboxylic acid methyl ester instead of 3-Bromo-benzoic acid methyl ester.

MS (ES⁺): 434 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 2.87 min.

### Example DI15

### 4-{3-[cis-4-Aminomethyl-4-(3-chloro-phenyl)-cyclohexyl]-2-oxo-imidazolidin-1-yl}-pyridine-2-carboxylic acid methyl ester hydrochloride

The title compound was prepared analogously as described in Example DI1, using 4-Bromo-pyridine-2-carboxylic acid methyl ester instead of 3-Bromo-benzoic acid methyl ester.

MS (ES⁺): 443 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 2.33 min.

### Example DI16

### 2-{3-[cis-4-Aminomethyl-4-(3-chloro-phenyl)-cyclohexyl]-2-oxo-imidazolidin-1-yl}-isonicotinic acid methyl ester hydrochloride

The title compound was prepared analogously as described in Example DI1, using 2-Bromo-isonicotinic acid methyl ester instead of 3-Bromo-benzoic acid methyl ester.

MS (ES⁺): 443 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 3.28 min.

### Example DI17

### 4-{3-[cis-4-Aminomethyl-4-(3-chloro-phenyl)-cyclohexyl]-2-oxo-imidazolidin-1-yl}-Pyridine-2-carboxylic acid hydrochloride

The title compound was prepared analogously as described in Example DI5, using 4-Bromo-pyridine-2-carboxylic acid methyl ester instead of 3-Bromo-benzoic acid methyl ester.

MS (ES⁺): 429 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 1.68 min.

### Example DI18

### 2-{3-[cis-4-Aminomethyl-4-(3-chloro-phenyl)-cyclohexyl]-2-oxo-imidazolidin-1-yl}-isonicotinic acid hydrochloride

The title compound was prepared analogously as described in Example DI5, using 2-Bromo-isonicotinic acid methyl ester instead of 3-Bromo-benzoic acid methyl ester.

MS (ES⁺): 429 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 2.43 min.

### Example DI19

### 3-{3-[cis-4-Aminomethyl-4-(3-chloro-phenyl)-cyclohexyl]-2-oxo-tetrahydro-pyrimidin-1-yl}-benzoic acid methyl ester hydrochloride

The title compound was prepared analogously as described in Example DI1 using (3-Amino-propyl)-carbamic acid benzyl ester instead of (2-Amino-ethyl)-carbamic acid benzyl ester hydrochloride.

MS (ES⁺): 456 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 3.29 min.

### Example DI20

### 4-{3-[cis-4-Aminomethyl-4-(3-chloro-phenyl)-cyclohexyl]-2-oxo-imidazolidin-1-yl}-benzamide hydrochloride

The title compound was prepared analogously as described in Example DI1, using 4-bromobenzamide instead of 3-Bromo-benzoic acid methyl ester.

MS (ES⁺): 427 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 2.54 min.

### Example DI21

### 2-{3-[cis-4-Aminomethyl-4-(3-chloro-phenyl)-cyclohexyl]-2-oxo-imidazolidin-1-yl}-benzoic acid methyl ester hydrochloride

The title compound was prepared analogously as described in Example DI1, using 2-Bromo-benzoic acid methyl ester instead of 3-Bromo-benzoic acid methyl ester.

MS (ES⁺): 442 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 3.10 min.

### Example DI22

### 6-{3-[cis-4-Aminomethyl-4-(3-chloro-phenyl)-cyclohexyl]-2-oxo-imidazolidin-1-yl}-Pyridine-2-carboxylic acid methyl ester hydrochloride

The title compound was prepared analogously as described in Example DI1, using 6-Bromo-pyridine-2-carboxylic acid methyl ester instead of 3-Bromo-benzoic acid methyl ester.

MS (ES⁺): 442 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 3.33 min.

### Example DI23

### 1-[cis-4-Aminomethyl-4-(3-chloro-phenyl)-cyclohexyl]-3-phenyl-tetrahydro-pyrimidin-2-one hydrochloride

The title compound was prepared analogously as described in Example DI19, using Bromobenzene instead of 3-Bromo-benzoic acid methyl ester.

MS (ES⁺): 398 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 3.08 min.

### Example DI24

### 4-{3-[cis-4-Aminomethyl-4-(3-chloro-phenyl)-cyclohexyl]-2-oxo-tetrahydro-pyrimidin-1-yl}-benzoic acid methyl ester hydrochloride

The title compound was prepared analogously as described in Example DI19, using 4-Bromo-benzoic acid methyl ester instead of 3-Bromo-benzoic acid methyl ester.

MS (ES⁺): 456 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 3.30 min.

### Example DI25

### 4-{3-[cis-4-Aminomethyl-4-(3-chloro-phenyl)-cyclohexyl]-2-oxo-imidazolidin-1-yl}-2-methyl-benzoic acid methyl ester hydrochloride

The title compound was prepared analogously as described in Example DI1, using 4-Bromo-2-methyl-benzoic acid methyl ester instead of 3-Bromo-benzoic acid methyl ester.

MS (ES⁺): 456 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 1.82 min.

### Example DI26

### 6-{3-[cis-4-Aminomethyl-4-(3-chloro-phenyl)-cyclohexyl]-2-oxo-imidazolidin-1-yl}-nicotinic acid methyl ester hydrochloride

The title compound was prepared analogously as described in Example DI1, using 6-Bromo-nicotinic acid methyl ester instead of 3-Bromo-benzoic acid methyl ester.

MS (ES⁺): 443 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 3.33 min.

### Example DI27

### 3-{3-[cis-4-Aminomethyl-4-(3-chloro-phenyl)-cyclohexyl]-2-oxo-imidazolidin-1-yl}-N,N-dimethyl-benzamide hydrochloride

The title compound was prepared analogously as described in Example DI1, using 3-Bromo-N,N-dimethyl-benzamide instead of 3-Bromo-benzoic acid methyl ester.

MS (ES⁺): 455 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 2.94 min.

### Example D128

### 4-{3-[cis-4-Aminomethyl-4-(3-chloro-phenyl)-cyclohexyl]-2-oxo-imidazolidin-1-yl}-benzonitrile hydrochloride

The title compound was prepared analogously as described in Example DI1, using 4-Bromo-benzonitrile instead of 3-Bromo-benzoic acid methyl ester.

MS (ES⁺): 409 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 3.51 min.

### Example DI29

### 3-{3-[cis-4-Aminomethyl-4-(3-chloro-phenyl)-cyclohexyl]-2-oxo-imidazolidin-1-yl}-benzonitrile hydrochloride

The title compound was prepared analogously as described in Example DI1, using 3-Bromo-benzonitrile instead of 3-Bromo-benzoic acid methyl ester.

MS (ES⁺): 409 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 3.56 min.

### Example DI30

### 2-{3-[cis-4-Aminomethyl-4-(3-chloro-phenyl)-cyclohexyl]-2-oxo-imidazolidin-1-yl}-benzoic acid hydrochloride

The title compound was prepared analogously as described in Example DI5, using 2-Bromo-benzoic acid methyl ester instead of 3-Bromo-benzoic acid methyl ester.

MS (ES⁺): 428 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 2.67 min.

### Example DI31

### 6-{3-[cis-4-Aminomethyl-4-(3-chloro-phenyl)-cyclohexyl]-2-oxo-imidazolidin-1-yl}-pyridine-2-carboxylic acid hydrochloride

The title compound was prepared analogously as described in Example DI5, using 6-Bromo-pyridine-2-carboxylic acid methyl ester instead of 3-Bromo-benzoic acid methyl ester.

MS (ES⁺): 429 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 2.35 min.

### Example DI32

### 3-{3-[cis-4-Aminomethyl-4-(3-chloro-phenyl)-cyclohexyl]-2-oxo-tetrahydro-pyrimidin-1-yl}-benzoic acid hydrochloride

The title compound was prepared analogously as described in Example DI5 using (3-Amino-propyl)-carbamic acid benzyl ester instead of (2-Amino-ethyl)-carbamic acid benzyl ester hydrochloride.

MS (ES⁺): 442 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 2.82 min.

### Example DI33

### 4-{3-[cis-4-Aminomethyl-4-(3-chloro-phenyl)-cyclohexyl]-2-oxo-tetrahydro-pyrimidin-1-yl}-benzoic acid hydrochloride

The title compound was prepared analogously as described in Example DI32, using 4-Bromo-benzoic acid methyl ester instead of 3-Bromo-benzoic acid methyl ester.

MS (ES⁺): 442 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 2.80 min.

### Example DI34

### 4-{3-[cis-4-Aminomethyl-4-(3-chloro-phenyl)-cyclohexyl]-2-oxo-imidazolidin-1-yl}-2-methyl-benzoic acid hydrochloride

The title compound was prepared analogously as described in Example DI5, using 4-Bromo-2-methyl-benzoic acid methyl ester instead of 3-Bromo-benzoic acid methyl ester.

MS (ES⁺): 442 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 3.09 min.

### Example DI35

### 6-{3-[cis-4-Aminomethyl-4-(3-chloro-phenyl)-cyclohexyl]-2-oxo-imidazolidin-1-yl}-nicotinic acid hydrochloride

The title compound was prepared analogously as described in Example DI5, using 6-Bromo-nicotinic acid methyl ester instead of 3-Bromo-benzoic acid methyl ester.

MS (ES⁺): 429 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 2.64 min.

### Example DI36

### 1-[cis-4-Aminomethyl-4-(3-chloro-phenyl)-cyclohexyl]-3-[4-(1H-tetrazol-5-yl)-phenyl]-imidazolidin-2-one hydrochloride

The title compound was prepared analogously as described in Example DI1, step A to J using 4-Bromo-benzonitrile instead of 3-Bromo-benzoic acid methyl ester to afford {1-(cis-3-Chloro-benzyl)-4-[3-(4-cyano-phenyl)-2-oxo-imidazolidin-1-yl]-cyclohexylmethyl}-carbamic acid tert-butyl ester followed by step.

### K) (1-(cis-3-Chloro-benzyl)-4-{2-oxo-3-[4-(1H-tetrazol-5-yl)-phenyl]-imidazolidin-1-yl}-cyclohexylmethyl)-carbamic acid tert-butyl ester

To a solution of {1-(cis-3-Chloro-benzyl)-4-[3-(4-cyano-phenyl)-2-oxo-imidazolidin-1-yl]-cyclohexylmethyl}-carbamic acid tert-butyl ester (75 mg, 0.147 mmol) in toluene (5 ml) and dimethylformamide (0.5 ml) were added Trimethylsilyl azide (300 μl, 2.21 mmol) and Tetrabutylammonium fluoride trihydrate (240 mg, 0.738 mmol). The mixture was treated with microwave for 2 h at 120° C. The reaction mixture was concentrated in vacuo. The residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were lyophilized in vacuo to give the title compound as a white solid.

MS (ES⁺): 569 [M+H2O]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 5.21 min.

### L) 1-[cis-4-Anninomethyl-4-(3-chloro-benzyl)-cyclohexyl]-3-[4-(1H-tetrazol-5-yl)-phenyl]-imidazolidin-2-one hydrochloride

To (1-(cis-3-Chloro-benzyl)-4-{2-oxo-3-[4-(1H-tetrazol-5-yl)-phenyl]-imidazolidin-1-yl}-cyclohexylmethyl)-carbamic acid tert-butyl ester (20 mg, 0.036 mmol) was added 4N hydrogen chloride solution in dioxane (5 ml). The reaction mixture stirred at room temperature for 2 h, then it was concentrated in vacuo. The residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were lyophilized in vacuo to give the formate salt of the title compound, which was dissolved in methanol and treated with an excess of 2M hydrogen chloride in methanol. Removal of the volatiles gave the title compound as a white solid.

MS (ES⁺): 452 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 2.86 min.

### Example DI37

### 1-[cis-4-Aminomethyl-4-(3-chloro-phenyl)-cyclohexyl]-3-[3-(1H-tetrazol-5-yl)-phenyl]-imidazolid in-2-one hydrochloride

The title compound was prepared analogously as described in Example DI36, using 3-Bromo-benzonitrile instead of 4-Bromo-benzonitrile.

MS (ES⁺): 452 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 3.30 min.

### Example DI38

### 3-{3-[cis-4-Aminomethyl-4-(3-chloro-phenyl)-cyclohexyl]-2-oxo-imidazolidin-1-yl}-N-methyl-benzamide hydrochloride

The title compound was prepared analogously as described in Example DI1, using 3-Bromo-N-methyl-benzamide instead of 3-Bromo-benzoic acid methyl ester.

MS (ES⁺): 441 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 2.79 min.

### Example DI39

### 4-{3-[cis-4-Aminomethyl-4-(3-chloro-phenyl)-cyclohexyl]-2-oxo-imidazolidin-1-yl}-N-methyl-benzamide hydrochloride

The title compound was prepared analogously as described in Example DI1, using 4-Bromo-N-methyl-benzamide instead of 3-Bromo-benzoic acid methyl ester.

MS (ES⁺): 441 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 2.65 min.

### Example DI40

### 4-{3-[cis-4-Aminomethyl-4-(3-chloro-phenyl)-cyclohexyl]-2-oxo-imidazolidin-1-yl}-N,N-dimethyl-benzamide hydrochloride

The title compound was prepared analogously as described in Example DI1, using 4-Bromo-N,N-dimethyl-benzamide instead of 3-Bromo-benzoic acid methyl ester.

MS (ES⁺): 455 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 2.85 min.

### Example DI41

### 5-{3-[cis-4-Aminomethyl-4-(3-chloro-phenyl)-cyclohexyl]-2-oxo-imidazolidin-1-yl}-pyridine-2-carboxylic acid methyl ester hydrochloride

The title compound was prepared analogously as described in Example DI1, using 5-Bromo-pyridine-2-carboxylic acid methyl ester instead of 3-Bromo-benzoic acid methyl ester.

MS (ES⁺): 443 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 2.81 min.

### Example DI42

### 4-{3-[cis-4-Aminomethyl-4-(3-chloro-phenyl)-cyclohexyl]-2-oxo-imidazolidin-1-yl}-3-methyl-benzoic acid methyl ester hydrochloride

The title compound was prepared analogously as described in Example DI1, using 4-Bromo-3-methyl-benzoic acid methyl ester instead of 3-Bromo-benzoic acid methyl ester.

MS (ES⁺): 456 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 3.32 min.

### Example DI43

### 3-{3-[cis-4-Aminomethyl-4-(3-chloro-phenyl)-cyclohexyl]-2-oxo-imidazolidin-1-yl}-benzamide hydrochloride

The title nornprnind was prapared analogously as described in Example DI1, using 3-Bromo-benzamide instead of 3-Bromo-benzoic acid methyl ester.

MS (ES⁺): 427 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 2.53 min.

### Example DI44

### 5-{3-[cis-4-Aminomethyl-4-(3-chloro-phenyl)-cyclohexyl]-2-oxo-imidazolidin-1-yl}-pyridine-2-carboxylic acid hydrochloride

The title compound was prepared analogously as described in Example DI1, using 5-Bromo-pyridine-2-carboxylic acid methyl ester instead of 3-Bromo-benzoic acid methyl ester.

MS (ES⁺): 429 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 2.04 min.

### Example DI45

### 4-{3-[cis-4-Aminomethyl-4-(3-chloro-phenyl)-cyclohexyl]-2-oxo-imidazolidin-1-yl}-3-methyl-benzoic acid hydrochloride

The title compound was prepared analogously as described in Example DI1, using 4-Bromo-3-methyl-benzoic acid methyl ester instead of 3-Bromo-benzoic acid methyl ester.

MS (ES⁺): 442 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 2.80 min.

### Example DI46

### 4-{(R)-3-[cis-4-Aminomethyl-4-(3-chloro-phenyl)-cyclohexyl]-5-methyl-2-oxo-imidazolidin-1-yl}-benzoic acid hydrochloride

The title compound was prepared analogously as described in Example DI5, using ((R)-2-Amino-1-methyl-ethyl)-carbamic acid benzyl ester instead of 3-Bromo-benzoic acid methyl ester.

MS (ES⁺): 442 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 3.05 min.

### Example DI47

### 4-{(S)-3-[cis-4-Aminomethyl-4-(3-chloro-phenyl)-cyclohexyl]-5-methyl-2-oxo-imidazolidin-1-yl}-benzoic acid hydrochloride

The title compound was prepared analogously as described in Example DI5, using ((S)-2-Amino-1-methyl-ethyl)-carbamic acid benzyl ester instead of 3-Bromo-benzoic acid methyl ester.

MS (ES⁺): 442 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 3.06 min.

### Example DI48

### 4-{(S)-3-[cis-4-Aminomethyl-4-(3-chloro-phenyl)-cyclohexyl]-4-methyl-2-oxo-imidazolidin-1-yl}-benzoic acid hydrochloride

The title compound was prepared analogously as described in Example DI5, using ((S)-2-Amino-propyl)-carbamic acid benzyl ester instead of 3-Bromo-benzoic acid methyl ester.

MS (ES⁺): 442 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 3.37 min.

### Example DI49

### 4-{(R)-3-[cis-4-Aminomethyl-4-(3-chloro-phenyl)-cyclohexyl]-4-methyl-2-oxo-imidazolidin-1-yl}-benzoic acid hydrochloride

The title compound was prepared analogously as described in Example DI5, using ((R)-2-Amino-propyl)-carbamic acid benzyl ester instead of 3-Bromo-benzoic acid methyl ester.

MS (ES⁺): 442 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 3.37 min.

### Example DJ1

### (S)-2-[trans-4-Aminomethyl-4-(3-chloro-phenyl)-cyclohexyl]-hexahydro-pyrrolo[1,2-a]pyrazine-1,4-dione

The title compound was prepared analogously as described in Example D1 step A to H using (S)-1-(2-Amino-acetyl)-pyrrolidine-2-carboxylic acid instead of 3-trifluoromethyl-5,6,7,8-tetrahydro-[1,2,4]triazolo[4,3-a]pyrazine to afford a mixture of (S)-1-{2-[4-(tert-Butoxycarbonylamino-methyl)-4-(cis-3-chloro-phenyl)-cyclohexylamino]-acetyl}-pyrrolidine-2-carboxylic acid and (S)-1-{2-[4-(tert-Butoxycarbonylamino-methyl)-4-(trans-3-chloro-phenyl)-cyclohexylamino]-acetyl}-pyrrolidine-2-carboxylic acid followed by step

### I) [1-(cis-3-Chloro-phenyl)-4-((S)-1,4-dioxo-hexahydro-pyrrolo[1,2-a]ovrazin-2-yl)-cyclohexylmethyl]-carbamic acid tert-butyl ester and [1-(trans-3-Chloro-phenyl)-4-((S)-1,4-dioxo-hexahydro-pyrrolo[1,2-a]ovrazin-2-yl)-cyclohexylmethyl]-carbamic acid tert-butyl ester

To a mixture of (S)-1-{2-[4-(tert-Butoxycarbonylamino-methyl)-4-(cis-3-chloro-phenyl)-cyclohexylamino]-acetyl}-pyrrolidine-2-carboxylic acid and (S)-1-{2-[4-(tert-Butoxycarbonylamino-methyl)-4-(trans-3-chloro-phenyl)-cyclohexylamino]-acetyl}-pyrrolidine-2-carboxylic acid (413 mg, 0.836 mmol) in dichloromethane (400 ml) was added 1-Hydroxybenzotriazole hydrate (452 mg, 3.34 mmol) and N-(3-Dimethylaminopropyl)-N′-ethylcarbodiimide hydrochloride (658 mg, 3.34 mmol). The solution was stirred at 0° C. for 30 minutes, then Triethylamine (1.16 ml, 8.36 mmol) was added dropwise at 0° C. The reaction mixture was stirred at room temperature for 16 h. To the reaction mixture was added some ice and 1M Hydrochloric acid until pH=2, then water was added and the product was extracted into dichloromethane. The organic layer was washed with saturated aqueous sodium bicarbonate solution and brine, then dried over sodium sulfate and concentrated in vacuo. The residue containing both diastereoisomers was purified and separated by prep. HPLC (InterChrom C18 ODB 10 μm 28×250 mm, flow 40 ml/min, 45 min method (0-2.5 min 20% ACN, 2.5-42.5 min 20-100% ACN, 42.5-45.0 min 100% ACN). Fractions containing the products were partitioned between dichloromethane and saturated aqueous sodium bicarbonate solution seperately. The organic layers were dried over sodium sulfate and concentrated in vacuo to give the title compounds as white solids.

MS (ES⁺): 500 [M+Na]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 3.24 min (trans) and 3.42 min (cis).

### J) (S)-2-[trans-4-Aminomethyl-4-(3-chloro-phenyl)-cyclohexyl]-hexahydro-pyrrolo[1,2-a]pyrazine-1,4-dione

Trifluoroacetic acid (640 μL) was added to a solution of [1-(trans-3-Chloro-phenyl)-4-((S)-1,4-dioxo-hexahydro-pyrrolo[1,2-a]pyrazin-2-ylycyclohexylmethyl]-carbamic acid tert-butyl ester (64 mg, 0.121 mmol) in dichloromethane (4 mL) and the reaction was stirred at room temperature for 2 h. The reaction mixture was concentrated in vacuo and the residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were partitioned between dichloromethane and saturated aqueous sodium bicarbonate solution. The organic layer was dried over sodium sulfate and concentrated in vacuo to give the title compound as a white solid.

MS (ES⁺): 376 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.52 min.

### Example DJ2

### (S)-2-[cis-4-Aminomethyl-4-(3-chloro-phenyl)-cyclohexyl]-hexahydro-pyrrolo[1,2-a]pyrazine-1,4-dione

The title compound was prepared analogously as described in Example DJ1 step J from [1-(cis-3-Chloro-phenyl)-4-((S)-1,4-dioxo-hexahydro-pyrrolo[1,2-a]pyrazin-2-yl)-cyclohexylmethyl]-carbamic acid tert-butyl ester.

MS (ES⁺): 376 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.77 min.

### Example DJ3

### (R)-1-[cis-4-Aminomethyl-4-(3-chloro-pherwl)-cyclohexyl]-3-benzyl-piperazine-2,5-dione

The title compound was prepared analogously as described in Example DJ2, using (R)-2-(2-Amino-acetylamino)-3-phenyl-propionic acid instead of (S)-1-(2-Amino-acetyl)-pyrrolidine-2-carboxylic acid.

MS (ES⁺): 426 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.02 min.

### Example DJ4

### (R)-1-[trans-4-Aminomethyl-4-(3-chloro-phenyl)-cyclohexyl]-3-benzyl-piperazine-2,5-dione

The title compound was prepared analogously as described in Example DJ1, using (R)-2-(2-Amino-acetylamino)-3-phenyl-propionic acid instead of (S)-1-(2-Amino-acetyl)-pyrrolidine-2-carboxylic acid.

MS (ES⁺): 426 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.82 min.

### Example E1

### N-[cis-4-(aminomethyl)-4-(3-chlorophenyl)cyclohexyl]pyridazine-3-carboxamide hydrochloride

The title compound was prepared according to Scheme E.

### A) cis-4-Aminomethyl-4-(3-chlorophenyl)-cyclohexanol

Borane tetrahydrofuran adduct (74.6 mL, 74.6 mmol of a 1M solution in THF) was carefully added to a solution of 1-(3-chlorophenyl)-4-oxo-cyclohexanecarbonitrile (4.36 g, 18.6 mmol) in tetrahydrofuran (120 mL) at 40° C. The reaction was then heated at reflux for 3 hours. After cooling, the reaction mixture was carefully quenched by the addition of 6M aqueous hydrochloric acid (200 ml), and was stirred at room temperature for 3 hours. The mixture was basified to pH10 with 1M aqueous sodium hydroxide and extracted with ethyl acetate (3×200 ml). The combined organics were washed with brine, dried (MgSO₄) and concentrated in vacuo to give the title compound as a white solid.

MS (ES⁺): 240, 242 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 1.96 min.

### B) [cis-1-(3-Chlorophenyl)-4-hydroxy-cyclohexylmethyl]-carbamic acid tert-butyl ester

tert-Butyloxycarbonyl anhydride (4.46 g, 20.0 mmol) was added to a solution of cis-4-aminomethyl-4-(3-chlorophenyl)-cyclohexanol (4.46 g, 18.6 mmol) and triethylamine (3.86 mL, 27.9 mmol) in tetrahydrofuran (50 mL) and the mixture stirred at room temperature for 3 hours. The reaction mixture was neutralized by the addition of 1M aqueous hydrochloric acid and the mixture extracted with ethyl acetate. The extracts were washed with water and brine, dried (MgSO₄) and concentrated in vacuo to give a yellow oil. The oil was purified by flash chromatography (NH2 anion exchange cartridge (50 g) using 20% ethyl acetate in cyclohexane as eluent) to give the title compound as a colourless oil.

MS (ES⁺): 340, 342 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 3.49 min.

### C) 2-Fluorobenzoic acid [trans-4-(tert-butoxycarbonylamino-methyl)-4-(3-chlorophenyl)-cyclohexyl]ester

Di-isopropyl-azodicarboxylate (2.55 mL, 12.94 mmol) was added to a solution of [cis-1-(3-chlorophenyl)-4-hydroxy-cyclohexylmethyl]-carbamic acid tert-butyl ester (2.0 g, 5.88 mmol), triphenylphosphine (3.4 g, 12.94 mmol) and 2-fluorobenzoic acid (1.98 g, 14.11 mmol) in tetrahydrofuran (30 mL) and the mixture was stirred at room temperature overnight. The mixture was concentrated in vacuo and the residue was purified by column chromatography (silica, using gradient elution with 0-20% ethyl acetate in cyclohexane) to give the title compound as a colourless oil that solidified on standing.

MS (ES⁺): 484 [M+Na]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 4.57 min.

### D) [trans-1-(3-Chlorophenyl)-4-hydroxy-cyclohexylmethyl]-carbamic acid tert-butyl ester

Sodium methoxide (528 mg, 9.77 mmol) was added to a solution of 2-fluorobenzoic acid [trans-4-(tert-butoxycarbonylamino-methyl)-4-(3-chlorophenyl)-cyclohexyl]ester (1.88 g, 4.07 mmol) in methanol (50 mL) and tetrahydrofuran (50 mL) and the mixture was stirred at room temperature overnight. The reaction mixture was concentrated in vacuo and the residue was dissolved in dichloromethane and water. 1M aqueous hydrochloric acid was added until the pH was 7 and the mixture was extracted with dichloromethane. The combined organic phases were washed with brine, dried (MgSO₄) and concentrated. The residue was purified by column chromatography (silica, using 1:1 cyclohexane:ethyl acetate as eluent) to afford the title compound as an oil.

T_{R} [HPLC, Phenomenex Luna 3 micron C18; 5-95% CH₃CN+0.1% Formic acid/H₂O+0.1% Formic acid for 5 min, flow 2.0 ml/min]: 3.32 min.

¹Hnmr [400 MHz, CDCl₃, tetramethylsilane as internal standard], δ 1.30 (2H, m), 1.39 (9H, s), 1.56 (2H, m), 1.88 (2H, m), 2.27 (2H, br d), 3.17 (2H, d), 3.72 (1H, m), 4.23 (1H, br t), 7.19-7.35 (4H, m).

### E) Methanesulphonic acid [trans-4-(tert-butoxycarbonylamino-methyl)-4-(3-chlorophenyl)-cyclohexyl]ester

Triethylamine (2.86 mL, 20.55 mmol) and methanesulphonyl chloride (0.8 mL, 10.3 mmol) were added to a solution of [trans-1-(3-chlorophenyl)-4-hydroxy-cyclohexylmethyl]-carbamic acid tert-butyl ester (1.4 g, 4.11 mmol) in dichloromethane (60 mL) with cooling to 0° C. The mixture was then stirred at room temperature for 2 hours. The mixture was washed with saturated aqueous ammonium chloride, saturated aqueous sodium bicarbonate and brine. After drying (MgSO₄), the volatiles were evaporated and the residue was purified by chromatography (silica, using 40% ethyl acetate in cyclohexane as eluent) to give the title compound as a colourless oil.

T_{R} [HPLC, Phenomenex Luna 3 micron C18; 5-95% CH₃CN+0.1% Formic acid/H₂O+0.1% Formic acid for 5 min, flow 2.0 ml/min]: 3.80 min.

¹Hnmr [400 MHz, CDCl₃, tetramethylsilane as internal standard], δ 1.39 (9H, s), 1.68 (4H, m), 2.05 (2H, m), 2.25 (2H, m), 2.97 (3H, s), 3.21 (2H, d), 4.24 (1H, br t), 4.77 (1H, m), 7.19-7.35 (4H, m).

### F) [cis-4-Azido-1-(3-chlorophenyl)-cyclohexylmethyl]-carbamic acid tert butyl ester

A mixture of sodium azide (125 mg, 1.91 mmol) and methanesulphonic acid [trans-4-(tert-butoxycarbonylamino-methyl)-4-(3-chlorophenyl)-cyclohexyl]ester (200 mg, 0.478 mmol) in dimethylformamide (10 mL) was stirred at 100° C. for 5 hours. After cooling, the mixture was diluted with ethyl acetate and washed with water and brine. The organic layer was dried (MgSO₄) and concentrated in vacuo to give the title compound as a colourless oil that was used directly in the next step.

T_{R} [HPLC, Phenomenex Luna 3 micron C18; 5-95% CH₃CN+0.1% Formic acid/H₂O+0.1% Formic acid for 5 min, flow 2.0 ml/min]: 4.48 min.

### G) [cis-4-Amino-1-(3-chlorophenyl)-cyclohexylmethyl]-carbamic acid tert butyl ester

Triphenylphosphine (2.2 g, 8.4 mmol) and water (0.8 mL) were added to a solution of [cis-4-azido-1-(3-chlorophenyl)-cyclohexylmethyl]-carbarnic acid tert butyl ester (1.54 g, 4.2 mmol) in toluene (20 mL) and the mixture was heated at 50° C. for 20 hours. The crude reaction mixture was applied to an SCX cartridge and eluted sequentially with dichloromethane, methanol and 2M ammonia in methanol. After combining and concentrating the fractions containing the desired product the residue was purified by column chromatography (silica, using gradient elution with 0-10% 2M ammonia in methanol/dichloromethane) to give the title compound as a colourless oil, which solidified on standing.

MS (ES⁺): 339 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 2.35 min.

### H) {cis-1-(3-Chlorophenyl)-4-[(pyridazine-3-carbonyl)-amino]-cyclohexylmethyl}-carbamic acid tert-butyl ester

[cis-4-Amino-1-(3-chlorophenyl)-cyclohexylmethyl]-carbamic acid tert butyl ester (50 mg, 0.148 mmol) was added to a solution of pyridazine-2-carboxylic acid (27 mg, 0.221 mmol), O-(7-azabenzotriazol-1-yl)-N,N,N′,N′-tetramethyluronium hexafluorophosphate (84 mg, 0.221 mmol) and diisopropylethylamine (78 μL) in dimethylformamide (1 mL) and the mixture stirred at room temperature for 2 days. The reaction was then diluted with ethyl acetate and washed repeatedly with water and brine. The organic layer was dried (MgSO₄) and concentrated in vacuo to give a yellow oil. The oil was purified by flash chromatography (silica, eluting sequentially with 1:1 cyclohexane:ethyl acetate and ethyl acetate) to give the title compound as a white solid.

MS (ES⁺): 467 [M+Na]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 3.65 min.

### I) N-[cis-4-(aminomethyl)-4-(3-chlorophenyl)cyclohexyl]pyridazine-3-carboxamide hydrochloride

Trifluoroacetic acid (0.6 mL) was added to a solution of {cis-1-(3-chlorophenyl)-4-[(pyridazine-3-carbonyl)-amino]-cyclohexylmethyl}-carbamic acid tert-butyl ester (60 mg, 0.135 mmol) in dichloromethane (6 mL) and the mixture was stirred at room temperature for 90 mins. After concentrating the mixture in vacuo the residue was purified by chromatography (SCX cartridge, eluting sequentially with dichloromethane, methanol and 0.5M ammonia in methanol) to give the free base of the title compound. The free base was dissolved in dichloromethane and treated with excess 1M hydrogen chloride in methanol. Evaporation and drying afforded the title compound as a white solid.

MS (ES⁺): 345, 347 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 5.18 min.

### Example E2

### N-[cis-4-(Aminomethyl)-4-(3-chlorophenyl)cyclohexyl]-1-benzofuran-2-carboxamide hydrochloride

The title compounds were prepared analogously as described in Example E1 using benzofuran-2-carboxylic acid instead of pyridazine-2-carboxylic acid.

MS (ES⁺): 383, 385 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 6.88 min.

### Example E3

### N-[cis-4-(Aminomethyl)-4-(3-chlorophenyl)cyclohexyl]-2-morpholin-4-ylacetamide hydrochloride

The title compounds were prepared analogously as described in Example E1 using morpholin-4-yl-acetic acid instead of pyridazine-2-carboxylic acid.

MS (ES⁺): 366, 368 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 3.43 min.

### Example E4

### 1-Acetyl-N-[cis-4-(aminomethyl)-4-(3-chlorophenyl)cyclohexyl]piperidine-4-carboxamide hydrochloride

The title compounds were prepared analogously as described in Example E1 using 1-acetyl-piperidine-4-carboxylic acid instead of pyridazine-2-carboxylic acid.

MS (ES⁺): 392, 394 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 4.72 min.

### Example E5

### N-[cis-4-(Aminomethyl)-4-(3-chlorophenyl)cyclohexyl]-2-pyridin-3-ylacetamide hydrochloride

The title compounds were prepared analogously as described in Example E1 using pyridine-3-yl-acetic acid instead of pyridazine-2-carboxylic acid.

MS (ES⁺): 358, 360 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 3.67 min.

### Example E6

### N-[cis-4-(Aminomethyl)-4-(3-chlorophenyl)cyclohexyl]-3-pyridin-3-ylpropanamide hydrochloride

The title compounds were prepared analogously as described in Example E1 using 3-pyridine-3-yl-propionic acid instead of pyridazine-2-carboxylic acid.

MS (ES⁺): 372, 374 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 3.68 min.

### Example E7

### N-[cis-4-(Aminomethyl)-4-(3-chlorophenyl)cyclohexyl]-3,5-dimethylisoxazole-4-carboxamide hydrochloride

The title compounds were prepared analogously as described in Example E1 using 3,5-dimethyl-isoxazole-4-carboxylic acid instead of pyridazine-2-carboxylic acid.

MS (ES⁺): 362, 364 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 5.38 min.

### Example E8

### N-[cis-4-(Aminomethyl)-4-(3-chlorophenyl)cyclohexyl]-1H-benzimidazole-5-carboxamide hydrochloride

The title compounds were prepared analogously as described in Example E1 using 1H-benzimidazole-5-carboxylic acid instead of pyridazine-2-carboxylic acid.

MS (ES⁺): 383, 385 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 4.16 min.

### Example E9

### N-[cis-4-(Aminomethyl)-4(3-chlorophenyl)cyclohexyl]-2-furamide hydrochloride

The title compounds were prepared analogously as described in Example E1 using 2-furoic acid instead of pyridazine-2-carboxylic acid.

MS (ES⁺): 333, 335 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 5.21 min.

### Example E10

### N-[cis-4-(Aminomethyl)-4-(3-chlorophenyl)cyclohexyl]benzamide hydrochloride

The title compounds were prepared analogously as described in Example E1 using benzoic acid instead of pyridazine-2-carboxylic acid.

MS (ES⁺): 343, 345 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 5.70 min.

### Example E11

### N-[cis-4-(Aminomethyl)-4-(3-chlorophenyl)cyclohexyl]pyrazine-2-carboxamide hydrochloride

The title compounds were prepared analogously as described in Example E1 using pyrazine-2-carboxylic acid instead of pyridazine-2-carboxylic acid.

MS (ES⁺): 345, 347 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 5.03 min.

### Example E12

### N-[cis-4-(Aminomethyl)-4-(3-chlorophenyl)cyclohexyl]-1,2,3-thiadiazole-4-carboxamide hydrochloride

The title compounds were prepared analogously as described in Example E1 using[1,2,3]thiadiazole-4-carboxylic acid instead of pyridazine-2-carboxylic acid.

MS (ES⁺): 351, 353 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 5.10 min.

### Example E13

### N-[cis-4-(Aminomethyl)-4-(3-chlorophenyl)cyclohexyl]-2-(4-methylphenoxy)acetamide hydrochloride

The title compounds were prepared analogously as described in Example E1 using para-tolyloxy-acetic acid instead of pyridazine-2-carboxylic acid.

MS (ES⁺): 387, 389 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 6.36 min.

### Example E14

### N-[cis-4-(Aminomethyl)-4-(3-chlorophenyl)cyclohexyl]-3-(phenylsulfonyl)propanamide hydrochloride

The title compounds were prepared analogously as described in Example E1 using 3-benzenesulfonyl-propionic acid instead of pyridazine-2-carboxylic acid.

MS (ES⁺): 435, 437 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 6.17 min.

### Example E15

### N-(2-{[cis-4-(Aminomethyl)-4-(3-chlorophenyl)cyclohexyl]amino}-2-oxoethyl)benzamide hydrochloride

The title compound was prepared analogously as described in Example E1 using N-benzoyl-glycine instead of pyridazine-3-carboxylic acid.

MS (ES⁺): 400, 402 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 6.00 min.

### Example E16

### N-(2-{[cis-4-(Aminomethyl)-4-(3-chlorophenyl)cyclohexyl]amino}-2-oxoethyncyclopropanecarboxamide hydrochloride

The title compound was prepared analogously as described in Example E1 using (cyclopropanecarbonyl-amino)-acetic acid instead of pyridazine-3-carboxylic acid.

MS (ES⁺): 364, 366 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 5.20 min.

### Example E17

### N-(2-{[cis-4-(Aminomethyl)-4-(3-chlorophenyl)cyclohexyl]amino}-2-oxoethyl)-2-furamide hydrochloride

The title compound was prepared analogously as described in Example E1 using [(furan-2-carbonyl)-amino]-acetic acid instead of pyridazine-3-carboxylic acid.

MS (ES⁺): 390, 392 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 5.55 min.

### Example E18

### N-[cis-4-(Aminomethyl)-4-(3-chlorophenyl)cyclohexyl]-4-morpholin-4-yl-4-oxobutanamide hydrochloride

The title compound was prepared analogously as described in Example E1 using 4-morpholin-4-yl-4-oxo-butyric acid instead of pyridazine-3-carboxylic acid.

MS (ES⁺): 408, 410 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 5.17 min.

### Example E19

### N-[cis-4-(Aminomethyl)-4-(3-chlorophenyl)cyclohexyl]pyridazine-4-carboxamide hydrochloride

The title compound was prepared analogously as described in Example E1 using pyridazine-4-carboxylic acid instead of pyridazine-3-carboxylic acid.

MS (ES⁻): 343, 345 [M−H]⁻.

T_{R} [HPLC, Higgins Clipeus 5 micron C18; 5-95% CH₃CN+0.1% Formic acid/H₂O+0.1% Formic acid for 20 min, flow 2.0 ml/min]: 5.16 min.

### Example E20

### N-[cis-4-(Aminomethyl)-4-(3-chlorophenyl)cyclohexyl]-2-(1-oxo-1,3-dihydro-2H-isoindol-2-yl)acetamide hydrochloride

The title compound was prepared analogously as described in Example E1 using (1-oxo-1,3-dihydro-isoindol-2-yl)-acetic acid instead of pyridazine-3-carboxylic acid.

MS (ES⁺): 412, 414 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 6.13 min.

### Example E21

### N-[cis-4-(Aminomethyl)-4-(3-chlorophenyl)cyclohexyl]-2-(1-oxo-1,3-dihydro-2H-isoindol-2-yl)acetamide hydrochloride

The title compound was prepared analogously as described in Example E1 using acetyl chloride instead of pyridazine-3-carboxylic acid.

MS (ES⁺): 281[M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 4.77 min.

### Example E22

### N-[cis-4-Aminomethyl-4-(3-chloro-phenyl)-cyclohexyl]-5-phenyl-nicotinamide dihydrochloride

The title compound was prepared analogously as described in Example E1 using 5-Phenylnicotinic acid instead of pyridazine-3-carboxylic acid.

MS (ES⁺): 420[M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 4.14 min.

### Example E23

### N-[cis-4-Aminomethyl-4-(3-chloro-phenyl)-cyclohexyl]-5-methyl-nicotinamide dihydrochloride

The title compound was prepared analogously as described in Example E1 using 5-Methylnicotinic acid instead of pyridazine-3-carboxylic acid.

MS (ES+): 358[M+H]+.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.38 min.

### Example E24

### 6-Acetylamino-N-[cis-4-aminomethyl-4-(3-chloro-phenyl)-cyclohexyl]-nicotinamide hydrochloride

The title compound was prepared analogously as described in Example E1 using 6-Acetylamino-nicotinic acid instead of pyridazine-3-carboxylic acid.

MS (ES+): 401 [M+H]+.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.57 min.

### Example E25

### N-[cis-4-Aminomethyl-4-(3-chloro-phenyl)-cyclohexyl]-6-methoxy-nicotinamide hydrochloride

The title compound was prepared analogously as described in Example E1 using 6-Methoxy-nicotinic acid instead of pyridazine-3-carboxylic acid.

MS (ES+): 374[M+H]+.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.87 min.

### Example E26

### N-[cis-4-Aminomethyl-4-(3-chloro-phenyl)-cyclohexyl]-6-morpholin-4-yl-nicotinamide dihydrochloride

The title compound was prepared analogously as described in Example E1 using 6-Morpholin-4-yl-nicotinic acid instead of pyridazine-3-carboxylic acid.

MS (ES⁺): 429[M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.48 min.

### Example E27

### N-[cis-4-Aminomethyl-4-(3-chloro-phenyl)-cyclohexyl]-3-formylamino-4-hydroxy-benzamide trifluoroacetate

The title compound was prepared analogously as described in Example E1 using Benzooxazole-5-carboxylic acid instead of pyridazine-3-carboxylic acid. The oxazole ring opened during purification.

MS (ES+): 402[M+H]+.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.54 min.

### Example E28

### 1-Isopropyl-2-trifluoromethyl-1H-benzoimidazole-5-carboxylic acid [cis-4-aminomethyl-4-(3-chloro-phenyl)-cyclohexyl]-amide hydrochloride

The title compound was prepared analogously as described in Example E1 using 1-Isopropyl-2-(trifluoromethyl)-1H-benzoimidazole-5-carboxylic acid instead of pyridazine-3-carboxylic acid.

MS (ES+): 493[M+H]+.

HPLC (Nucleosil C-18HD 4×70 mm 3 μm, 8 min method (0-6 min 5-100% ACN, 6.0-7.5 min 100% ACN, 7.5-8.0 min 100-5% ACN): 4.58 min.

### Example E29

### 1-Isopropyl-1H-benzotriazole-5-carboxylic acid [cis-4-aminomethyl-4-(3-chloro-phenyl)-cyclohexyl]-amide hydrochloride

The title compound was prepared analogously as described in Example E1 using 1-Isopropyl-1H-benzotriazole-5-carboxylic acid instead of pyridazine-3-carboxylic acid.

MS (ES+): 426[M+H]+.

HPLC (Nucleosil C-18HD 4×70 mm 3 μm, 8 min method (0-6 min 5-100% ACN, 6.0-7.5 min 100% ACN, 7.5-8.0 min 100-5% ACN): 4.15 min.

### Example E30

### 1-Isopropyl-1H-pyrazolo[3,4-b]pyridine-5-carboxylic acid [cis-4-aminomethyl-4-(3-chloro-phenyl)-cyclohexyl]-amide hydrochloride

The title compound was prepared analogously as described in Example E1 using 1-Isopropyl-1H-pyrazolo[3,4-b]pyridine-5-carboxylic acid instead of pyridazine-3-carboxylic acid.

MS (ES+): 426[M+H]+.

HPLC (Nucleosil C-18HD 4×70 mm 3 μm, 8 min method (0-6 min 5-100% ACN, 6.0-7.5 min 100% ACN, 7.5-8.0 min 100-5% ACN): 4.18 min.

### Example E31

### 1-Methyl-1H-indole-5-carboxylic acid [cis-4-aminomethyl-4-(3-chloro-phenyl)-cyclohexyl]-amide

The title compound was prepared analogously as described in Example E1 using 1-Methyl-1H-indole-5-carboxylic acid instead of pyridazine-3-carboxylic acid.

MS (ES⁺): 396[M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 4.20 min.

### Example E32

### N-[cis-4-Aminomethyl-4-(3-chloro-phenyl)-cyclohexyl]-nicotinamide hydrochloride

The title compound was prepared analogously as described in Example E1 using Nicotinic acid instead of pyridazine-3-carboxylic acid.

MS (ES⁺): 344[M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 1.98 min.

### Example E33

### N-[cis-4-Aminomethyl-4-(3-chloro-phenyl)-cyclohexyl]-isonicotinamide hydrochloride

The title compound was prepared analogously as described in Example E1 using Isonicotinic acid instead of pyridazine-3-carboxylic acid.

MS (ES+): 344[M+H]+.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.30 min.

### Example E34

### 2-Acetylamino-N-[cis-4-aminomethyl-4-(3-chloro-phenyl)-cyclohexyl]-isonicotinamide hydrochloride

The title compound was prepared analogously as described in Example E1 using 2-Acetylaminoisonicotinic acid instead of pyridazine-3-carboxylic acid.

MS (ES+): 401 [M+H]+.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.55 min.

### Example E35

### 6-Amino-N-[cis-4-aminomethyl-4-(3-chloro-phenyl)-cyclohexyl]-nicotinamide hydrochloride

The title compound was prepared analogously as described in Example E1 using 6-Aminonicotinic acid instead of pyridazine-3-carboxylic acid.

MS (ES+): 359[M+H]+.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.30 min.

### Example E36

### N-[cis-4-Aminomethyl-4-(3-chloro-phenyl)-cyclohexyl]-6-trifluoromethyl-nicotinamide hydrochloride

The title compound was prepared analogously as described in Example E1 using 6-(Trifluoromethyl)-nicotinic acid instead of pyridazine-3-carboxylic acid.

MS (ES+): 412[M+H]+.

HPLC (Nucleosil C-18HD 4×70 mm 3 μm, 8 min method (0-6 min 5-100% ACN, 6.0-7.5 min 100% ACN, 7.5-8.0 min 100-5% ACN): 4.23 min.

### Example E37

### 3,4,5,6-Tetrahydro-2H-[1,2′]bipyridinyl-4′-carboxylic acid [cis-4-aminomethyl-4-(3-chloro-phenyl)-cyclohexyl]-amide dihydrochloride

The title compound was prepared analogously as described in Example E1 using 3,4,5,6-Tetrahydro-2H-[1,2′]bipyridinyl-4′-carboxylic acid instead of pyridazine-3-carboxylic acid.

MS (ES+): 427[M+H]+.

HPLC (Nucleosil C-18HD 4×70 mm 3 μm, 8 min method (0-6 min 5-100% ACN, 6.0-7.5 min 100% ACN, 7.5-8.0 min 100-5% ACN): 2.03 min.

### Example E38

### N-[cis-4-Aminomethyl-4-(3-chloro-phenyl)-cyclohexyl]-6-methyl-nicotinamide hydrochloride

The title compound was prepared analogously as described in Example E1 using 6-Methylnicotinic acid instead of pyridazine-3-carboxylic acid.

MS (ES+): 358[M+H]+.

HPLC (Nucleosil C-18HD 4×70 mm 3 μm, 8 min method (0-6 min 20-100% ACN, 6.0-7.5 min 100% ACN, 7.5-8.0 min 100-20% ACN): 1.87 min.

### Example E39

### N-[4-Aminomethyl-4-(3-chloro-phenyl)-cyclohexyl]-2-methoxy-isonicotinamide hydrochloride

The title compound was prepared analogously as described in Example E1 using 2-Methoxy-isonicotinic acid instead of pyridazine-3-carboxylic acid.

MS (ES+): 374[M+H]+.

HPLC (Nucleosil C-18HD 4×70 mm 3 μm, 8 min method (0-6 min 5-100% ACN, 6.0-7.5 min 100% ACN, 7.5-8.0 min 100-5% ACN): 2.23 min.

### Example E40

### N-[4-Aminomethyl-4-(3-chloro-phenyl)-cyclohexyl]-6-(4-methyl-piperazin-1-yl)-nicotinamide dihydrochloride

The title compound was prepared analogously as described in Example E1 using 6-(4-methyl-piperazin-1-yl)-nicotinic acid instead of pyridazine-3-carboxylic acid.

MS (ES+): 442[M+H]+.

HPLC (Nucleosil C-18HD 4×70 mm 3 μm, 8 min method (0-6 min 5-100% ACN, 6.0-7.5 min 100% ACN, 7.5-8.0 min 100-5% ACN): 1.84 min.

### Example E41

### 1-Cyclopropyl-1H-benzoimidazole-5-carboxylic acid [4-aminomethyl-4-(3-chloro-phenyl)-cyclohexyl]-amide hydrochloride

The title compound was prepared analogously as described in Example E1 using 1-Cyclopropyl-1H-benzoimidazole-5-carboxylic acid instead of pyridazine-3-carboxylic acid.

MS (ES+): 423[M+H]+.

HPLC (Nucleosil C-18HD 4×70 mm 3 μm, 8 min method (0-6 min 20-100% ACN, 6.0-7.5 min 100% ACN, 7.5-8.0 min 100-20% ACN): 2.09 min.

### Example E42

### 3-Isopropyl-isoxazolo[5,4-b]pyridine-5-carboxylic acid [4-aminomethyl-4-(3-chloro-phenyl)-cyclohexyl]-amide hydrochloride

The title compound was prepared analogously as described in Example E1 using 3-Isopropyl-isoxazolo[5,4-b]pyridine-5-carboxylic acid instead of pyridazine-3-carboxylic acid.

MS (ES+): 427[M+H]+.

HPLC (Nucleosil C-18HD 4×70 mm 3 μm, 8 min method (0-6 min 5-100% ACN, 6.0-7.5 min 100% ACN, 7.5-8.0 min 100-5% ACN): 4.35 min.

### Example E43

### 6-(Acetylamino-methyl)-N-[4-aminomethyl-4-(3-chloro-phenyl)-cyclohexyl]-nicotinamide hydrochloride

The title compound was prepared analogously as described in Example E1 using 6-(Acetylamino-methyl)-nicotinic acid instead of pyridazine-3-carboxylic acid.

MS (ES+): 415[M+H]+.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.37 min.

### Example E44

### N-[4-Aminomethyl-4-(3-chloro-phenyl)-cyclohexyl]-6-[1,2,4]triazol-1-yl-nicotinamide hydrochloride

The title compound was prepared analogously as described in Example E1 using 6-[1,2,4]triazol-1-yl-nicotinic acid instead of pyridazine-3-carboxylic acid.

MS (ES+): 411[M+H]+.

HPLC (Nucleosil C-18HD 4×70 mm 3 μm, 8 min method (0-6 min 5-100% ACN, 6.0-7.5 min 100% ACN, 7.5-8.0 min 100-5% ACN):

### Example E45

### N-[4-Aminomethyl-4-(3-chloro-phenyl)-cyclohexyl]-3-methanesulfonyl-benzamide hydrochloride

The title compound was prepared analogously as described in Example E1 using 3-Methanesulfonyl-benzoic acid instead of pyridazine-3-carboxylic acid.

MS (ES+): 421[M+H]+,

HPLC (Nucleosil C-18HD 4×70 mm 3 μm, 8 min method (0-6 min 5-100% ACN, 6.0-7.5 min 100% ACN, 7.5-8.0 min 100-5% ACN): 3.74 min.

### Example E46

### N-[4-Aminomethyl-4-(3-chloro-phenyl)-cyclohexyl]-4-methanesulfonyl-benzamide hydrochloride

The title compound was prepared analogously as described in Example E1 using 4-Methanesulfonyl-benzoic acid instead of pyridazine-3-carboxylic acid.

MS (ES+): 421 [M+H]+.

HPLC (Nucleosil C-18HD 4×70 mm 3 μm, 8 min method (0-6 min 5-100% ACN, 6.0-7.5 min 100% ACN, 7.5-8.0 min 100-6% ACN): 3.76 min.

### Example E47

### 5-Methanesulfonyl-thiophene-2-carboxylic acid [4-aminomethyl-4-(3-chloro-phenyl)-cyclohexyl]-amide hydrochloride

The title compound was prepared analogously as described in Example E1 using 5-Methanesulfonyl-thiophene-2-carboxylic acid instead of pyridazine-3-carboxylic acid.

MS (ES+): 427[M+H]+.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.92 min.

### Example E48

### 2-(3-Methanesulfonyl-phenyl)-pyrimidine-4-carboxylic acid [4-aminomethyl-4-(3-chloro-phenyl)-cyclohexyl]-amide hydrochloride

The title compound was prepared analogously as described in Example E1 using 2-(3-Methanesulfonyl-phenyl)-pyrimidine-4-carboxylic acid instead of pyridazine-3-carboxylic acid.

MS (ES+): 499[M+H]+.

HPLC (Nucleosil C-18HD 4×70 mm 3 μm, 8 min method (0-6 min 5-100% ACN, 6.0-7.5 min 100% ACN, 7.5-8.0 min 100-5% ACN): 4.32 min.

### Example E49

### N-[4-Aminomethyl-4-(3-chloro-phenyl)-cyclohexyl]-2-(3-methanesulfonylamino-phenyl)-acetamide hydrochloride

The title compound was prepared analogously as described in Example E1 using 2-(3-methanesulfonylamino-phenyl)-acetic acid instead of pyridazine-3-carboxylic acid.

MS (ES+): 450[M+H]+.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.69 min.

### Example E50

### 4-Aminomethyl-4-(3-chloro-phenyl)-cyclohexylamine hydrochloride

The title compound was prepared analogously as described in Example E1, step A to G followed by step

### H) 4-Aminomethyl-4-(3-chloro-phenyl)-cyclohexylamine hydrochloride

Trifluoroacetic acid (271 μl) was added to a solution of [4-Amino-1-(3-chloro-phenyl)-cyclohexylmethyl]-carbamic acid tert-butyl ester (120 mg, 0.354 mmol) in dichloromethane (3 mL). The reaction mixture was stirred at room temperature for 1 h. The mixture was concentrated in vacuo. The residue was dissolved in dioxane and treated with an excess of 4M hydrogen chloride in dioxane. Lyophilization of the mixture gave the title compound as a white solid.

MS (ES⁺): 240 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.28 min.

### Example EA1

### 2-[cis-4-Aminomethyl-4-(3-chloro-phenyl)-cyclohexyl]-isoindole-1,3-dione

The title compound was prepared according to Scheme E.

The title compound was prepared analogously as described in Example E1, step A to G followed by step

### H) N-[4-(tert-Butoxycarbonylamino-methyl)-4-(cis-3-chloro-phenyl)-cyclohexyl]-phthalamic acid

To a solution of [4-Amino-1-(cis-3-chloro-phenyl)-cyclohexylmethyl]-carbamic acid tert-butyl ester (100 mg, 0.274 mmol) in chloroform (2 mL) was added phthalic anhydride (55 mg, 0.37 mmol). The reaction mixture was stirred at 70° C. for 16 h. The mixture was concentrated in vacuo. The residue was purified by flash chromatography (Silica cartridge) using gradient elution from 100% cyclohexane to 100% ethylacetate, then dichloromethane/methanol 8:2. Fractions containing the product were concentrated in vacuo to give the title compound as a white solid.

MS (ES⁺): 432 [M+H-tBu]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 3.50 min.

### I) [1-(cis-3-Chloro-phenyl)-4-(1,3-dioxo-1,3-dihydro-isoindol-2-yl)-cyclohexylmethyl]-carbamic acid tert-butyl ester

To a solution of N-[4-(tert-Butoxycarbonylamino-methyl)-4-(cis-3-chloro-phenyl)-cyclohexyl]-phthalamic acid (100 mg, 0.191 mmol) in acetonitrile (2 mL) were added (Benzotriazol-1-yloxy)tripyrrolidinophosphonium hexafluorophosphate (119 mg, 0.229 mmol) and triethylamine (32 μl, 0.229 mmol). The reaction mixture was stirred at room temperature for 4 h. The mixture was partitioned between dichloromethane and saturated aqueous sodium bicarbonate solution. The organic layer was dried and concentrated in vacuo. The residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were partitioned between dichloromethane and saturated aqueous sodium bicarbonate solution. The organic layer was dried and concentrated in vacuo to give the title compound as a white solid.

MS (ES⁺): 469 [M+H]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 4.39 min.

### J) 2-[cis-4-Aminomethyl-4-(3-chloro-phenyl)-cyclohexyl]-isoindole-1,3-dione

Trifluoroacetic acid (500 μl) was added to a solution of [1-(cis-3-Chloro-phenyl)-4-(1,3-dioxo-1,3-dihydro-isoindol-2-yl)-cyclohexylmethyl]-carbamic acid tert-butyl ester (50 mg, 0.099 mmol) in dichloromethane (1 mL). The reaction mixture was stirred at room temperature for 2 h. The mixture was partitioned between dichloromethane and saturated aqueous sodium bicarbonate solution. The organic layer was dried and concentrated in vacuo. The residue was purified by prep. HPLC (Waters SunFire Prep C18 ODB Spm 19×50 mm, flow 20 ml/min, 15 min method (0-2.5 min 5% ACN, 2.5-12.5 min 5-100% ACN, 12.5-15.0 min 100% ACN). Fractions containing the product were partitioned between dichloromethane and saturated aqueous sodium bicarbonate solution. The organic layer was dried and concentrated in vacuo to give the title compound as a white solid.

MS (ES⁺): 369 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.81 min.

### Example EB1

### 4-[cis-4-Aminomethyl-4-(3-chloro-phenyl)-cyclohexyl]-3-oxo-piperazine-1-carboxylic acid benzyl ester

The title compound was prepared analogously as described in Example E1, step A to G followed by step

### H) (Benzyloxycarbonyl-{2-[4-(tert-butoxycarbonylamino-methyl)-4-(cis-3-chloro-phenyl)-cyclohexylamino]-ethyl}-amino)-acetic acid ethyl ester

To a solution of [4-Amino-1-(cis-3-chloro-phenyl)cyclohexylmethyl]-carbamic acid tert-butyl ester (406 mg, 1.20 mmol) in 1,2-Dichloroethane (3 mL) were added [Benzyloxycarbonyl-(2-oxo-ethyl)-amino]-acetic acid ethyl ester (300 mg, 1.00 mmol) and acetic acid (57 μl, 1.4 mmol). The mixture was stirred at room temperature for 1 h, then Sodium triacetoxyborohydride was added. The reaction mixture was stirred at room temperature for 16 h. The mixture was partitioned between dichloromethane and saturated aqueous sodium bicarbonate solution. The organic layer was dried and concentrated in vacuo. The residue was purified by prep. HPLC (Waters SunFire Prep C18 ODB Spm 30×100 mm, flow 40 ml/min, 45 min method (0-2.5 min 20% ACN, 2.5-42.5 min 20-100% ACN, 42.5-45.0 min 100% ACN). Fractions containing the product were partitioned between dichloromethane and saturated aqueous sodium bicarbonate solution. The organic layer was dried and concentrated in vacuo to give the title compound as a white solid.

MS (ES⁺): 602 [M+H]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 3.49 min.

### I) 4-[4-(tert-Butoxycarbonylamino-methyl)-4-(cis-3-chloro-phenyl)-cyclohexyl]-3-oxo-piperazine-1-carboxylic acid benzyl ester

A solution of (Benzyloxycarbonyl-{2-[4-(tert-butoxycarbonylamino-methyl)-4-(cis-3-chloro-phenyl)-cyclohexylamino]-ethyl}-amino)acetic acid ethyl ester (50 mg, 0.083 mmol) in a mixture of toluene (1 ml), n-Butanol (1 ml) and acetic acid (215 μl) was treated with microwave at 150° C. for 40 minutes. The mixture was concentrated in vacuo. The residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were partitioned between dichloromethane and saturated aqueous sodium bicarbonate solution. The organic layer was dried and concentrated in vacuo to give the title compound as a white solid.

MS (ES⁺): 580 [M+Na]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 4.01 min.

### J) 4-[cis-4-Aminomethyl-4-(3-chloro-phenyl)-cyclohexyl]-3-oxo-piperazine-1-carboxylic acid benzyl ester

Trifluoroacetic acid (177 μl) was added to a solution of 4-[4-(tert-Butoxycarbonylamino-methyl)-4-(cis-3-chloro-phenyl)-cyclohexyl]-3-oxo-piperazine-1-carboxylic acid benzyl ester (21 mg, 0.035 mmol) in dichloromethane (1 mL). The reaction mixture was stirred at room temperature for 2 h. The mixture was concentrated in vacuo. The residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were partitioned between dichloromethane and saturated aqueous sodium bicarbonate solution. The organic layer was dried and concentrated in vacuo. The residue was treated with diethylether. After removal of the etheric phase with a pipette, the residue was dissolved in Methanol and treated with an excess of 2M Hydrochloric acid in methanol. The volatiles were evaporated, then the residue was dissolved in dioxane and lyophilized to give the title compound as a white solid.

MS (ES⁺): 456 [M+H]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 2.70 min.

### Example F1

### N-[cis-4-(aminomethyl)-4-(3-chlorophenyl)cyclohexyl]-3,5-dimethylisoxazole-4-sulfonamide and N-[trans-4-(aminomethyl)-4-(3-chlorophenyl)cyclohexyl]-3,5-dimethylisoxazole-4-sulfonamide

The title compounds were prepared according to Scheme F.

### A) A mixture of [trans-1-(3-chlorophenyl)-4-hydroxy-cyclohexylmethyl]-carbamic acid tert-butyl ester and [cis-1-(3-chlorophenyl)-4-hydroxy-cyclohexylmethyl]-carbamic acid tert-butyl ester

Sodium borohydride (361 mg, 9.6 mmol) was added to a solution of 1-(3-chlorophenyl)-4-oxo-cyclohexanecarbonitrile (1.61 g, 4.78 mmol) in tetrahydrofuran (20 mL) and the mixture was stirred at room temperature for 2 hours. The mixture was diluted with water and extracted with ethyl acetate (2×150 ml). The combined organic extracts were washed with brine, dried (MgSO₄) and concentrated in vacuo. The residue was purified by flash chromatography (silica cartridge (50 g), using a gradient elution from 5% ethyl acetate in cyclohexane to 40% ethyl acetate in cyclohexane) to give a mixture of the title compounds as a colourless oil.

MS (ES⁺): 284 [M+H-tBu]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 3.30 and 3.44 min.

### B) A mixture of methanesulphonic acid [trans-4-(tert-butoxycarbonylamino-methyl)-4-(3-chlorophenyl)-cyclohexyl]ester and methanesulphonic acid [cis-4-(tert-butoxycarbonylamino-methyl)-4-(3-chlorophenyl)-cyclohexyl]ester

Triethylamine (1.15 mL, 8.3 mmol) and methane sulphonyl chloride (0.32 mL, 4.16 mmol) were added to a solution of a mixture of [trans-1-(3-chlorophenyl)-4-hydroxy-cyclohexylmethyl]-carbamic acid tert-butyl ester and [cis-1-(3-chlorophenyl)-4-hydroxy-cyclohexylmethyl]-carbamic acid tert-butyl ester (564 mg, 1.66 mmol) in dichloromethane (10 mL) and the mixture was stirred at room temperature for 2 hours. The mixture was partitioned between aqueous ammonium chloride and dichloromethane (2×150 ml). The combined organics were washed with aqueous sodium hydrogen carbonate and brine, dried (MgSO₄) and concentrated. The residue was purified by flash chromatography (Silica cartridge (50 g), using a gradient elution from 10% ethyl acetate in cyclohexane to 30% ethyl acetate in cyclohexane) to give a mixture of the title compounds as a colourless gum.

T_{R} [HPLC, Phenomenex Luna 3 micron C18; 5-95% CH₃CN+0.1% Formic acid/H₂O+0.1% Formic acid for 5 min, flow 2.0 ml/min]: 3.96 min.

### C) A mixture of [trans-4-azido-1-(3-chlorophenyl)-cyclohexylmethyl]-carbamic acid tert-butyl ester and [cis-4-azido-1-(3-chlorophenyl)-cyclohexylmethyl]-carbamic acid tert-butyl ester

Sodium azide (1.72 g, 26.51 mmol) was added to a solution of a mixture of methanesulphonic acid [trans-4-(tert-butoxycarbonylamino-methyl)-4-(3-chlorophenyl)-cyclohexyl]ester and methanesulphonic acid [cis-4-(tert-butoxycarbonylamino-methyl)-4-(3-chlorophenyl)-cyclohexyl]ester (2.77 g, 66.3 mmol) in dimethylformamide (140 mL) and the reaction mixture was heated at 100° C. for 5 hours. After cooling, the mixture was diluted with water and extracted with ethyl acetate (4×150 ml), the combined extracts were washed with water and brine, and dried (MgSO₄). Concentration in vacuo afforded a mixture of the title compounds as a yellow oil, which was used directly in the next step.

T_{R} [HPLC, Phenomenex Luna 3 micron C18; 5-95% CH₃CN+0.1% Formic acid/H₂O+0.1% Formic acid for 5 min, flow 2.0 ml/min]: 4.40 and 4.46 min.

### D) A mixture of [trans-4-amino-1-(3-chlorophenyl)-cyclohexylmethyl]-carbamic acid tert-butyl ester and [cis-4-amino-1-(3-chlorophenyl)-cyclohexylmethyl]-carbamic acid tert-butyl ester

Triphenylphosphine (3.44 g, 13.1 mmol) and water (1.18 mL) were added to a mixture of [trans-4-azido-1-(3-chlorophenyl)-cyclohexylmethyl]-carbamic acid tert butyl ester and [cis-4-azido-1-(3-chlorophenyl)-cyclohexylmethyl]-carbamic acid tert butyl ester (2.41 g, 6.57 mmol) in toluene (40 mL) and the reaction mixture was heated at 50° C. overnight. After cooling, the reaction mixture was concentrated in vacuo to remove most of the solvent. The residual solution was initially purified by ion exchange chromatography (SCX-2 column (25 g), eluting sequentially with dichloromethane, 1:1 dichloromethane:methanol, methanol and 2M ammonia in methanol). Fractions containing the desired products were further purified by flash chromatography (silica (70 g), eluting with 200:2:0.5 dichloromethane:ethanol:(aq)ammonia to 200:8:1 dichloromethane:ethanol:(aq)ammonia) the mixture of title compounds as a yellow oil.

MS (ES⁺): 285 [M+H-tBu]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 2.28 and 2.38 min.

### E) A mixture of [trans-1-(3-chlorophenyl)-4-(3,5-dimethylisoxazole-4-sulfonylamino)-cyclohexylmethyl]-carbamic acid tert-butyl ester and [cis-1-(3-chlorophenyl)-4-(3,5-dimethylisoxazole-4-sulfonylamino)-cyclohexylmethyl]-carbamic acid tert-butyl ester

N-Methyl morpholine (80 μL, 0.7 mmol) and 3,5-dimethyl-isoxazole-4-sulphonyl chloride (102 mg, 0.52 mmol) were added to a stirred mixture of [trans-4-amino-1-(3-chlorophenyl)-cyclohexylmethyl]-carbamic acid tert-butyl ester and [cis-4-amino-1-(3-chlorophenyl)-cyclohexylmethyl]-carbamic acid tert-butyl ester (118 mg, 0.35 mmol) in dichloromethane (3 mL) and stirring was continued for 3 hours. The mixture was washed with 1M hydrochloric acid (2 mL) and evaporated. The residue was purified by flash chromatography (silica (5 g), eluting with pentane then pentane:diethyl ether 1:1) to give the title compounds as a colourless oil.

MS (ES⁺): 498, 500 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 3.93 and 4.11 min.

### F) A mixture of N-[cis-4-(aminomethyl)-4-(3-chlorophenyl)cyclohexyl]-3,5-dimethylisoxazole-4-sulfonamide and N-[trans-4-(aminomethyl)-4-(3-chlorophenyl)cyclohexyl]-3,5-dimethylisoxazole-4-sulfonamide

A mixture of [trans-1-(3-chlorophenyl)-4-(3,5-dimethylisoxazole-4-sulfonylamino)-cyclohexylmethyl]-carbamic acid tert-butyl ester and [cis-1-(3-chlorophenyl)-4-(3,5-dimethylisoxazole-4-sulfonylamino)-cyclohexylmethyl]-carbamic acid tert-butyl ester (137 mg, 0.28 mmol) trifluoroacetic acid (0.5 mL) and dichloromethane (2 mL) was stirred for 2 h, then blown down to dryness. The residue was chromatographed (SCX cartridge (5 g) eluting sequentially with dichloromethane, dichloromethane:methanol 1:1, and dichloromethane:methanol 1:1 with 5% aq. ammonia) to give a colourless oil. The oil was further purified by chromatography (silica, (5 g) eluting sequentially with dichloromethane:ethanol:ammonia, 400:8:1, 200:8:1 then 100:8:1) to give a mixture of the title compounds in the form of a white solid.

MS (ES⁺): 398, 400 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 6.20 and 6.89 min.

### Example F2

### N-[cis-4-(Aminomethyl)-4-(3-chlorophenyl)cyclohexyl]thiophene-2-sulfonamide hydrochloride and N-[trans-4-(aminomethyl)-4-(3-chlorophenyl)cyclohexyl]thiophene-2-sulfonamide hydrochloride

The title compounds were prepared analogously as described in Example F1 using thiophene-2-sulfonyl chloride instead of 3,5-dimethyl-isoxazole-4-sulphonyl chloride. The hydrochloride salts were prepared by treatment with excess hydrochloric acid in methanol followed by drying. The title compounds were obtained as a mixture.

MS (ES⁺): 385, 387 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 6.82 min.

### Example F3

### N-[cis-4-(Aminomethyl)-4-(3-chlorophenyl)cyclohexyl]pyridine-3-sulfonamide hydrochloride and N-[trans-4-(aminomethyl)-4-(3-chlorophenyl)cyclohexyl]pyridine-3-sulfonamide hydrochloride

The title compounds were prepared analogously as described in Example F1 using pyridine-3-sulfonyl chloride instead of 3,5-dimethyl-isoxazole-4-sulphonyl chloride. The hydrochloride salts were prepared by treatment with excess hydrochloric acid in methanol followed by drying. The title compounds were obtained as a mixture.

MS (ES⁺): 380, 382 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 5.63 min.

### Example F4

### N-[cis-4-(Aminomethyl)-4-(3-chlorophenyl)cyclohexyl]methanesulfonamide hydrochloride and N-[trans-4-(aminomethyl)-4-(3-chlorophenyl)cyclohexyl]methanesulfonamide hydrochloride

The title compounds were prepared analogously as described in Example F1 using methane-sulfonyl chloride instead of 3,5-dimethyl-isoxazole-4-sulphonyl chloride. The hydrochloride salts were prepared by treatment with excess hydrochloric acid in methanol followed by drying. The title compounds were obtained as a mixture.

MS (ES⁺): 317, 319 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 4.30 and 5.00 min.

### Example F5

### N-[cis-4-(Aminomethyl)-4-(3-chlorophenyl)cyclohexyl]-4-(trifluoromethyl)benzenesulfonamide hydrochloride and N-[trans-4-(aminomethyl)-4-(3-chlorophenyl)cyclohexyl]-4-(trifluoromethyl)benzenesulfonamide hydrochloride

The title compounds were prepared analogously as described in Example F1 using 4-(trifluoromethyl)-benzene-sulfonyl chloride instead of 3,5-dimethyl-isoxazole-4-sulphonyl chloride. The diastereomers were separated by mass directed preparative HPLC. The hydrochloride salts were prepared by treatment with excess hydrochloric acid in methanol followed by drying.

Cis diastereisomer

MS (ES⁺): 447, 449 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 7.52 min. Trans diastereoisomer

MS (ES⁺): 447, 449 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 6.94 min.

### Example F6

### N-[cis-4-(Aminomethyl)-4-(3-chlorophenyl)cyclohexyl]-1-methyl-1H-imidazole-4-sulfonamide hydrochloride and N-[trans-4-(aminomethyl)-4-(3-chlorophenyl)cyclohexyl]-1-methyl-1H-imidazole-4-sulfonamide hydrochloride

The title compounds were prepared analogously as described in Example F1 using 1-methyl-1H-imidazole-4-sulfonyl chloride instead of 3,5-dimethyl-isoxazole-4-sulphonyl chloride. The hydrochloride salts were prepared by treatment with excess hydrochloric acid in methanol followed by drying. The title compounds were obtained as a mixture.

MS (ES⁺): 383, 385 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 4.34 and 6.16 min.

### Example F7

### N-[cis-4-(Aminomethyl)-4-(3-chlorophenyl)cyclohexyl]-6-chloroimidazo[2,1-b][1,3]thiazole-5-sulfonamide hydrochloride and N-[trans-4-(aminomethyl)-4-(3-chlorophenyl)cyclohexyl]-6-chloroimidazo[2,1-b][1,3]thiazole-5-sulfonamide hydrochloride

The title compounds were prepared analogously as described in Example F1 using 6-chloro-imidazo[2,1-b]thiazole-5-sulfonyl chloride instead of 3,5-dimethyl-isoxazole-4-sulphonyl chloride. The hydrochloride salts were prepared by treatment with excess hydrochloric acid in methanol followed by drying. The title compounds were obtained as a mixture.

MS (ES⁺): 459, 461,463 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 6.75 and 7.25 min.

### Example F8

### N-[cis-4-(Aminomethyl)-4-(3-chlorophenyl)cyclohexyl]-4-(trifluoromethoxy)benzenesulfonamide hydrochloride and N-[trans-4-(aminomethyl)-4-(3-chlorophenyl)cyclohexyl]-4-(trifluoromethoxy)benzenesulfonamide hydrochloride

The title compounds were prepared analogously as described in Example F1 using 4-trifluoromethoxy-benzenesulfonyl chloride instead of 3,5-dimethyl-isoxazole-4-sulphonyl chloride. The hydrochloride salts were prepared by treatment with excess hydrochloric acid in methanol followed by drying. The title compounds were obtained as a mixture.

MS (ES⁺): 463, 465 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 4.62 min.

### Example F9

### N-[cis-4-(Aminomethyl)-4-(3-chlorophenyl)cyclohexyl]-2-(trifluoromethyl)benzenesulfonamide hydrochloride and N-[trans-4-(aminomethyl)-4-(3-chlorophenyl)cyclohexyl]-2-(trifluoromethyl)benzenesulfonamide hydrochloride

The title compounds were prepared analogously as described in Example F1 using 2-trifluoromethyl-benzenesulfonyl chloride instead of 3,5-dimethyl-isoxazole-4-sulphonyl chloride. The hydrochloride salts were prepared by treatment with excess hydrochloric acid in methanol followed by drying. The title compounds were obtained as a mixture.

MS (ES⁺): 447, 449 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 7.18 and 7.59 min.

### Example F10

### N-[cis-4-(Aminomethyl)-4-(3-chlorophenyl)cyclohexyl]-5-(phenylsulfonyl)thiophene-2-sulfonamide hydrochloride and N-[trans-4-(aminomethyl)-4-(3-chlorophenyl)cyclohexyl]-5-(phenylsulfonyl)thiophene-2-sulfonamide hydrochloride

The title compounds were prepared analogously as described in Example F1 using 5-(phenylsulfonyl)-thiophene-2-sulfonyl chloride instead of 3,5-dimethyl-isoxazole-4-sulphonyl chloride. The hydrochloride salts were prepared by treatment with excess hydrochloric acid in methanol followed by drying. The title compounds were obtained as a mixture.

MS (ES⁺): 525, 527 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 7.55 and 8.00 min.

### Example F11

### N-[cis-4-(Aminomethyl)-4-(3-chlorophenyl)cyclohexyl]-1-phenylmethanesulfonamide hydrochloride and N-[trans-4-(aminomethyl)-4-(3-chlorophenyl)cyclohexyl]-1-phenylmethanesulfonamide hydrochloride

The title compounds were prepared analogously as described in Example F1 using benzylsulfonyl chloride instead of 3,5-dimethyl-isoxazole-4-sulphonyl chloride. The hydrochloride salts were prepared by treatment with excess hydrochloric acid in methanol followed by drying. The title compounds were obtained as a mixture.

MS (ES⁺): 393, 395[M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 6.54 and 7.09 min.

### Example F12

### N-[cis-4-(Aminomethyl)-4-(3-chlorophenyl)cyclohexyl]-2-(1,3-dioxo-1,3-dihydro-2H-isoindol-2-yl)ethanesulfonamide hydrochloride and N-[trans-4-(aminomethyl)-4-(3-chlorophenyl)cyclohexyl]-2-(1,3-dioxo-1,3-dihydro-2H-isoindol-2-yl)ethanesulfonamide hydrochloride

The title compounds were prepared analogously as described in Example F1 using 2-(1,3-dioxo-1,3-dihydro-isoindol-2-yl)-ethanesulfonyl chloride instead of 3,5-dimethyl-isoxazole-4-sulphonyl chloride. The hydrochloride salts were prepared by treatment with excess hydrochloric acid in methanol followed by drying. The title compounds were obtained as a mixture.

MS (ES⁺): 476, 478 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 6.63 and 7.01 min.

### Example G1

### N-[cis-4-(aminomethyl)-4-(3-chlorophenyl)cyclohexyl]benzenesulfonamide hydrochloride and N-[trans-4-(aminomethyl)-4-(3-chlorophenyl)cyclohexyl]benzenesulfonamide hydrochloride

The title compounds were prepared according to Scheme G.

### A) A mixture of N-[cis-4-(3-chlorophenyl)-4-cyano-cyclohexyl]-benzenesulfonamide and N-[trans-4-(3-chlorophenyl)-4-cyano-cyclohexyl]-benzenesulfonamide

Sodium cyanoborohydride (128 mg, 2.03 mmol) was added to a stirred mixture of ammonium chloride (453 mg, 8.47 mmol), 3 A molecular sieves and 1-(3-chlorophenyl)-4-oxo-cyclohexanecarbonitrile (396 mg, 1.69 mmol) in methanol (5 mL) at 0° C. and stirring in an ice bath was continued over night. Triethylamine (0.47 mL, 3.39 mmol) and benzenesulfonyl chloride (0.65 mL, 5.1 mmol) were added and the mixture was stirred for a further 2 hours. The reaction mixture was neutralized with 1M hydrochloric acid and extracted with ethyl acetate. The aqueous phase was basified with aqueous sodium bicarbonate and extracted with ethyl acetate. The organics were combined, washed with water and brine, dried (MgSO4), and concentrated. The residue was purified by flash chromatography (Silica (10 g), eluting with 10% ethyl acetate in cyclohexane) to give a mixture of the title compounds as a pale yellow oil.

MS (ES⁺): 373 [M−H]⁻.

T_{R} [HPLC, Phenomenex Luna 3 micron C18; 5-95% CH₃CN+0.1% Formic acid/H₂O+0.1% Formic acid for 5 min, flow 2.0 ml/min]: 3.93 min.

### B) N-[cis-4-(aminomethyl)-4-(3-chlorophenyl)cyclohexyl]benzenesulfonamide hydrochloride and N-[trans-4-(aminomethyl)-4-(3-chlorophenyl)cyclohexyl]benzenesulfonamide hydrochloride

Borane-tetrahydrofuran complex (600 μL, 0.6 mmol of a 1M solution in tetrahydrofuran) was added to a solution of a mixture of N-[cis-4-(3-chlorophenyl)-4-cyano-cyclohexyl]-benzenesulfonamide and N-Wans-4-(3-chlorophenyl)-4-cyano-cyclohexyli-benzenesulfonamide (50 mg, 0.134 mmol) in tetrahydrofuran (3 mL) under a nitrogen atmosphere. The reaction mixture was refluxed for 4 hours. Carefully, concentrated sulphuric acid (1.5 ml) was added and the mixture was refluxed for a further 2 hours. After cooling to room temperature the mixture was basified with aqueous sodium hydroxide. The mixture was extracted with dichloromethane (3×20 ml), the combined extracts were washed with water and brine, dried (MgSO₄) and concentrated. The residue was purified by chromatography (SCX-2 column (5 g), eluting sequentially with dichloromethane, ethyl acetate, methanol and 2M ammonia in methanol), and then by flash chromatography (Silica (2 g), eluting with 10:4:4:0.5 dichloromethane:ethanol:methanol:aq. ammonia). Finally, purification by reversed phase HPLC ( ) afforded the separated title compounds which were converted to hydrochloride salts (Example F2).

Cis diastereoisomer

MS (ES⁺): 379, 381 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 6.38 min.

Trans diastereoisomer

MS (ES⁺): 379, 381 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 5.60 min.

### Example H1

### cis-4-(Aminomethyl)-4-(3-chlorophenyl)-N-[2-(trifluoromethyl)benzyl]cyclohexanecarboxamide hydrochloride

The title compound was prepared according to Scheme H.

### A) 1-(3-Chlorophenol)-4-methoxymethylene-cyclohexanecarbonitrile

Lithium bis(trimethylsilylamide) (12.8 mL, 12.8 mmol of a 1M solution in tetrahydrofuran) was added dropwise to a suspension of (methoxymethyl)triphenylphosphonium chloride (4.53 g, 12.8 mmol) in tetrahydrofuran (13 mL) under an argon atmosphere at 0° C. After 30 min, the suspension was added to a solution of 1-(3-chlorophenyl)-4-oxo-cyclohexanecarbonitrile (2.0 g, 8.55 mmol) in tetrahydrofuran (19 mL) with cooling to 0° C. After 5 h of stirring at 0° C., water was carefully added and the mixture was extracted with diethyl ether. The combined extracts were washed with water, dried (Na₂SO₄), and concentrated in vacuo. The residue was purified by flash chromatography (silica, gradient elution with cyclohexane to cyclohexane/ethyl acetate 92:8) to give the title compound as a white solid.

¹Hnmr [400 MHz, CDCl₃, tetramethylsilane as internal standard], δ 1.76 (2H, m), 2.18 (4H, m), 2.47 (2H, m), 2.95 (2H, m), 3.58 (3H, s), 5.87 (1H, br. s), 7.25-7.35 (2H, m), 7.38 (1H, m), 7.45 (1H, br. s).

### B) cis-1-(3-Chlorophenol)-4-formyl-cyclohexanecarbonitrile

Hydrochloric acid (1M, 2 mL) was added to a solution of 1-(3-chlorophenyl)-4-methoxymethylene-cyclohexanecarbonitrile (549 mg, 2.09 mmol) in acetonitrile (4.8 mL) and the mixture was stirred at room temperature for 16 hours. The mixture was neutralised by the addition of saturated aqueous sodium bicarbonate and extracted with diethyl ether. The extracts were washed with water (twice), dried (Na₂SO₄), filtered and concentrated in vacuo. The residue was purified by flash chromatography (silica, gradient elution with cyclohexane to cyclohexane/ethyl acetate 99:1 to 82:18) to give trans-1-(3-chlorophenyl)-4-formyl-cyclohexanecarbonitrile as the minor isomer and the title compound, cis-1-(3-chlorophenyl)-4-formyl-cyclohexanecarbonitrile, as the major isomer.

Trans diastereoisomer:

¹Hnmr [400 MHz, CDCl₃, tetramethylsilane as internal standard], δ 1.72-1.92 (2H, m), 2.00-2.25 (4H, m), 2.2-2.93 (2H, m), 2.69 (1H, m), 7.25-7.36 (3H, m), 7.42 (1H, br. s), 9.76 (1H, s).

Cis diastereoisomer

¹Hnmr [400 MHz, CDCl₃, tetramethylsilane as internal-standard], δ 1.75-1.98 (4H, m), 2.03-2.41 (5H, m), 7.27-7.44 (3H, m), 7.48 (1H, br. s), 9.68 (1H, s).

### C) cis-4-(3-Chlorophenyl)-4-cyano-cyclohexanecarboxylic acid

A mixture of sodium chlorite (245 mg, 2.16 mmol) and sodium dihydrogenphosphate monohydrate (381 mg, 2.70 mmol) in water (8 mL) was added to a suspension of cis-1-(3-chlorophenyl)-4-formyl-cyclohexanecarbonitrile (268 mg, 1.08 mmol) in a solution of 2-methyl-2-butene (458 μL, 4.32 mmol) in tert-butanol (6 mL). After stirring for 1 hour, the mixture was acidified with 1M hydrochloric acid and extracted with ethyl acetate. The extracts were dried (Na₂SO₄) and concentrated in vacuo to give the title compound as a white solid.

MS (ES⁻): 262 [M−H]⁻.

T_{R} [HPLC, Phenomenex Luna 3 micron C18; 5-95% CH₃CN+0.1% Formic acid/H₂O+0.1% Formic acid for 5 min, flow 2.0 ml/min]: 3.27 min.

### D) cis-4-(3-Chlorophenyl)-cyano-cyclohexanecarboxylic acid 2-trifluoromethyl-benzylamide

Diisopropylethylamine (146 μL, 0.85 mmol) and then O-(7-azabenzotriazol-1-yl)-N,N,N′,N′-tetramethyluronium hexafluorophosphate (119 mg, 0.31 mmol) were added to a solution of cis-4-(3-chlorophenyl)-4-cyano-cyclohexanecarboxylic acid (75 cmg, 0.28 mmol) and 2-(trifluoromethyl)benzylamine (54.8 mg, 0.31 mmol) in dimethylformamide (2.5 mL). After stirring for 20 h, saturated aqueous sodium bicarbonate was added and the mixture was extracted with dichloromethane. The extracts were washed with water, filtered through a hydrophobic membrane and concentrated in vacuo. The residue was purified by flash chromatography (silica, gradient elution with cyclohexane/ethyl acetate 9:1 to 75:25) to give the title compound as a white solid.

MS (ES⁺): 421 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 3.97 min.

### E) cis-4-(Aminomethyl)-4-(3-chlorophenyl)-N-[2-(trifluoromethyl)benzyl]cyclohexanecarboxamide hydrochloride

cis-4-(3-Chlorophenyl)-cyano-cyclohexanecarboxylic acid 2-trifluoromethyl-benzylamide (92 mg, 0.21 mmol) and cobalt II chloride hexahydrate (104 mg, 2.18 mmol) were dissolved in methanol (7 mL) under a nitrogen atmosphere. The mixture was stirred and sodium borohydride (83 mg, 2.18 mmol) was added portionwise, allowing the effervescence to subside between additions; then the mixture was stirred for 16 hours. The reaction was adjusted to pH=2 by the addition of 1M hydrochloric acid at 0° C. After stirring for 10 min the mixture was basified with saturated aqueous sodium bicarbonate and extracted thoroughly with ethyl acetate. The combined extracts were washed with water, dried (Na₂SO₄), and concentrated in vacuo. The residue was purified by flash chromatography (silica, gradient elution with dichloromethane/2M ammonia in methanol 98.5:1.5 to 96:4) to give the free base of the title compound. The hydrochloride salt was prepared by dissolution of the free base in methanol, treatment with a small excess of hydrochloric acid and evaporation of volatiles. After drying, the title compound was obtained as an off-white solid.

MS (ES⁺): 425, 427 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 7.29 min.

### Example H2

### 1-{[cis-4-(Aminomethyl)-4-(3-chlorophenyl)cyclohexyl]carbonyl}-1,4-diazepan-5-one hydrochloride

The title compound was prepared analogously as described in Example H1 using[1,4]diazepan-5-one instead of 2-(trifluoromethyl)benzylamine.

MS (ES⁺): 364, 366 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 5.02 min.

### Example H3

### 1-(cis-1-(3-Chlorophenyl)-4-{[3-trifluoromethyl)-5,6-dihydro[1,2,4]triazolo[4,3-a]pyrazin-7(8H)-yl]carbonyl}cyclohexyl)methanamine hydrochloride

The title compound was prepared analogously as described in Example H1 using 3-trifluoromethyl-5,6,7,8-tetrahydro-[1,2,4]triazolo[4,3-a]pyrazine instead of 2-(trifluoromethyl)benzylamine.

MS (ES⁺): 442, 444 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 6.14 min.

### Example H4

### 1-(1-{[cis-4-(Aminomethyl)-4-(3-chlorophenyl)cyclohexyl]carbonyl}piperidin-4-yl)-1,3-dihydro-2H-benzimidazol-2-one hydrochloride

The title compound was prepared analogously as described in Example H1 using 1-piperidin-4-yl-1,3-dihydro-benzimidazol-2-one instead of 2-(trifluoromethyl)benzylamine.

MS (ES⁺): 467, 469 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 6.74 min.

### Example H5

### cis-4-(Aminomethyl)-4-(3-chlorophenyl)-N-(pyridin-3-ylmethyl)cyclohexanecarboxamide hydrochloride

The title compound was prepared analogously as described in Example H1 using C-pyridin-3-yl-methylamine instead of 2-(trifluoromethyl)benzylamine.

MS (ES⁺): 358, 360 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 3.75 min.

### Example H6

### cis-4-(Aminomethyl)-4(3-chlorophenyl)-N-(1-ethyl-1H-pyrazol-5-yl)cyclohexanecarboxamide hydrochloride

The title compound was prepared analogously as described in Example H1 using 2-ethyl-2H-pyrazol-3-ylamine instead of 2-(trifluoromethyl)benzylamine.

MS (ES⁺): 361, 363 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 5.48 min.

### Example H7

### 1-[cis-1-(3-chlorophenyl)-4-(3,4,6,7-tetrahydro-5H-imidazo[4,5-c]pyridin-5-ylcarbonyl)cyclohexyl]methanamine dihydrochloride and 1-[trans-1-(3-chlorophenyl)-4-(3,4,6,7-tetrahydro-5H-imidazo[4,5-c]pyridin-5-ylcarbonyl)cyclohexyl]methanamine dihydrochloride

The title compounds were prepared analogously as described in Example H1 using 4,5,6,7-tetrahydro-3H-imidazo[4,5-c]pyridine instead of 2-(trifluoromethyl)benzylamine, and a mixture of cis- and trans-4-(3-chlorophenyl)-4-cyano-cyclohexanecarboxylic acid.

Cis diastereoisomer:

MS (ES⁺): 373, 375 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 1.30 min.

Trans diastereoisomer:

MS (ES⁺): 373, 375 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 4.26 min.

### Example I1

### 1-(cis-1-(3-chlorophenyl)-4-{[3-(trifluoromethyl)-5,6-dihydro[1,2,4]triazolo[4,3-a]pyrazin-7(8H)-yl]methyl}cyclohexyl)methanamine dihydrochloride

Borane-dimethylsulphide complex (236 μL, 2.49 mmol) was added dropwise during 20 min to a solution of 1-(3-chlorophenyl)-4-(3-trifluoromethyl-5,6-dihydro-8H-[1,2,4]triazolo[4,3-a]pyrazine-7-carbonyl)-cyclohexanecarbonitrile (156 mg, 0.35 mmol) in tetrahydrofuran (9 mL) under an argon atmosphere. The mixture was warmed to 60° C. under reflux. After stirring for 18 hours, the reaction was allowed to cool to room temperature and was then cooled to 0° C. Water (7 mL) was added and then the reaction was heated at 60° C. for 3 hours. After cooling, the mixture was extracted with ethyl acetate, the extracts were dried (Na₂SO₄) and concentrated in vacuo. The residue was purified by flash chromatography (silica cartridge eluting with dichloromethane then dichloromehane/2M ammonia in methanol), and then by reversed phase preparative HPLC (15% to 95% CH₃CN in H2O at 1 mL/min, flow 5 mL/min). Appropriate fractions were concentrated in vacuo and treated with hydrogen chloride in methanol. Evaporation of the volatiles in vacuo and final drying under high vacuum afforded the title compound as an amorphous solid.

MS (ES⁺): 428, 430 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 6.02 min.

### Example J1

### 6-(Aminomethyl)-6-(3-chlorophenyl)-2-methyl-5,6,7,8-tetrahydroquinazolin-4(1H)-one

The title compound was prepared according to Scheme J.

### A) 6-(3-Chlorophenyl)-2-methyl-4-oxo-3,4,5,6,7,8-hexahydro-quinazoline-6-carbonitrile

A mixture of 5-(3-chlorophenyl)-5-cyano-2-oxo-cyclohexanecarboxylic acid methyl ester (100 mg, 0.34 mmol) acetamidine hydrochloride (58 mg, 0.60 mmol) and potassium carbonate (96 mg, 0.69 mmol) in methanol (2 mL) was heated at 75° C. for 18 hours. After cooling to room temperature, the mixture was acidified to pH=7 with concentrated hydrochloric acid and extracted with ethyl acetate. The extracts were washed with water and brine, dried (Na₂SO₄), and concentrated in vacuo to give the title compound as an off-white solid.

MS (ES⁺): 300, 302 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 2.67 min.

### B) 6-(Aminomethyl)-6-(3-chlorophenyl)-2-methyl-5,6,7,8-tetrahydroquinazolin-4(1H)-one

6-(3-Chlorophenyl)-2-methyl-4-oxo-3,4,5,6,7,8-hexahydro-quinazoline-6-carbonitrile (55 mg, 0.18 mmol) and cobalt II chloride hexahydrate (88 mg, 0.36 mmol) were dissolved in methanol (2.75 mL) under a nitrogen atmosphere. The mixture was stirred and sodium borohydride (49 mg, 1.27 mmol) was added portionwise, allowing the effervescence to subside between additions; then the mixture was stirred for 20 hours. The reaction mixture was filtered through diatomaceous earth, the pad rinsed with methanol and the washings and filtrate were concentrated in vacuo. The residue was dissolved in ethyl acetate, washed with water and the organic phase dried (Na₂SO₄). After concentration, the residue was purified on an ion exchange cartridge (SCX-2 cartridge, eluting with dichloromethane/methanol 1:1 then 2M ammonia in methanol). The residue was further purified by flash chromatography (silica, gradient elution with dichloromethane/2M ammonia in methanol 98.5:1.5 to 93:7) to give the title compound as a colourless oil.

MS (ES⁺): 304, 306 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 3.72 min.

### Example J2

### 6-(Aminomethyl)-6-(3-chlorophenyl)-2-phenyl-5,6,7,8-tetrahydroquinazolin-4(1H)-one

The title compound was prepared analogously as described in Example J1 using benzamidine hydrochloride instead of acetamidine hydrochloride.

MS (ES⁺): 366, 368 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 6.11 min.

### Example K1

### N-[trans-4-(Aminomethyl)-4-(3-chlorophenyl)cyclohexyl]pyridazine-3-carboxamide Hydrochloride

The title compound was prepared according to Scheme K.

### A) Methanesulfonic acid 4-(tert-butoxycarbonylamino-methyl)-4-(3-chloro-phenyl)-cyclohexyl ester

Triethylamine (2.3 mL, 16.5 mmol) and methanesulphonyl chloride (0.64 mL, 8.24 mmol) were added to a solution of [cis-1-(3-chlorophenyl)-4-hydroxy-cyclohexylmethyl]-carbamic acid tert-butyl ester [Example E1] (1.4 g, 4.12 mmol) in dichloromethane (65 mL) at 0° C. The mixture was stirred at room temperature for 2 hours. The solution was washed with aqueous ammonium chloride, aqueous sodium bicarbonate and brine, then dried and concentrated in vacuo to give a yellow oil. The oil was purified by flash chromatography (silica, eluting with 40% ethyl acetate in cyclohexane) to give the title compound as a colourless oil.

MS (ES⁺): 440 [M+Na]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 3.45 min.

### B) [trans-4-Azido-1-(3-chloro-phenyl)-cyclohexylmethyl]-carbamic acid tert-butyl ester

Sodium azide (809 mg, 12.44 mmol) was added to a solution of methanesulfonic acid 4-(tert-butoxycarbonylamino-methyl)-4-(3-chloro-phenyl)-cyclohexyl ester (1.3 g, 3.11 mmol) in dimethylformamide (80 mL) and the mixture was stirred at 100° C. for 5 hours. After cooling, the mixture was diluted with ethyl acetate and washed with water and brine. The organic layer was dried and concentrated in vacuo to give the title compound as a colourless oil.

MS (ES⁺): 406 [M+H acetonitrile adduct]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 4.38 min.

### C) [trans-4-Amino-1-(3-chlorophenyl)-cyclohexylmethyl]-carbamic acid tert butyl ester

Triphenylphosphine (1.41 g, 5.37 mmol) and water (0.5 mL) were added to a solution of [trans-4-azido-1-(3-chlorophenyl)-cyclohexylmethyl]-carbamic acid tert butyl ester (980 mg, 2.68 mmol) in toluene (20 mL) and the mixture was heated at 50° C. for 20 hours. The crude reaction mixture was purified twice on an ion exchange column (SCX, eluting sequentially with dichloromethane, methanol and 2M ammonia in methanol) to give the title compound as a colourless oil, which solidified on standing.

MS (ES⁺): 339 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 2.14 min.

### D) {trans-1-(3-Chlorophenyl)-4-[(pyridazine-3-carbonyl)-amino]-cyclohexylmethyl}-carbamic acid tert-butyl ester

[trans-4-Amino-1-(3-chlorophenyl)-cyclohexylmethyl]-carbamic acid tert butyl ester (100 mg, 0.295 mmol) was added to a solution of pyridazine-3-carboxylic acid (55 mg, 0.442 mmol), O-(7-azabenzotriazol-1-yl)-N,N,N′,N′-tetramethyluronium hexafluorophosphate (168 mg, 0.442 mmol) and diisopropylethylamine (0.16 mL, 0.885 mmol) in dimethylformamide (2 mL) and the mixture stirred at room temperature for 20 hours. The reaction was concentrated in vacuo and partitioned between ethyl acetate and aqueous sodium bicarbonate. After passing through a phase separator the organic layer was dried, and evaporated to give a yellow oil. The oil was purified by flash chromatography (silica, gradient elution from 50-75% thyl acetate in cyclohexane) to give the title compound as a white solid.

MS (ES⁺): 467 [M+Na]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 3.22 min.

### E) N-[trans-4-(Aminomethyl)-4-(3-chlorophenyl)cyclohexyl]pyridazine-3-carboxamide hydrochloride

Trifluoroacetic acid (1 mL) was added to a solution of {trans-1-(3-chlorophenyl)-4-[(pyridazine-3-carbonyl)-amino]-cyclohexylmethyl}-carbamic acid tert-butyl ester (75 mg, 0.168 mmol) in dichloromethane (10 mL) and the mixture was stirred at room temperature for 90 mins. The reaction mixture was purified by chromatography (SCX cartridge, eluting sequentially with dichloromethane, methanol and 0.5M ammonia in methanol) to give the free base of the title compound. The free base was dissolved in methanol and treated with excess 1M hydrogen chloride in methanol. Evaporation and drying afforded the title compound as a white solid.

MS (ES⁺): 345 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 5.24 min.

### Example K2

### 1-Acetyl-N-[trans-4-(aminomethyl)-4-(3-chlorophenyl)cyclohexyl]piperidine-4-carboxamide hydrochloride

The title compound was prepared analogously as described in Example K1 using 1-acetyl-piperidine-4-carboxylic acid instead pyridazine-3-carboxylic acid.

MS (ES⁺): 392 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 5.11 min.

### Example K3

### N-[trans-4-(aminomethyl)-4-(3-chlorophenyl)cyclohexyl]-2-furamide hydrochloride

The title compound was prepared analogously as described in Example K1 using furan-2-carboxylic acid instead pyridazine-3-carboxylic acid.

MS (ES⁺): 333 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 5.93 min.

### Example L1

### cis-4-(Aminomethyl)-4-(3-chlorophenyl)-N-[(4-phenyl-1H-pyrazol-5-yl)methyl]cyclohexanamine hydrochloride

The title compound was prepared according to Scheme L

### A) {cis-1-(3-Chloro-phenyl)-4-[(4-phenyl-2H-pyrazol-3-ylmethyl)-amino]-cyclohexylmethyl}-carbamic acid tea-butyl ester

A mixture of [cis-4-amino-1-(3-chlorophenyl)-cyclohexylmethyl]-carbamic acid ten butyl ester [Example E1] (130 mg, 0.384 mmol) and 4-phenyl-2H-pyrazole-3-carbaldehyde (68 mg, 0.394 mmol) in acetic acid (0.3 mL) and dichloromethane (2 mL) was stirred in the presence of 4 Å molecular sieves for 30 min. Sodium triacetoxyborohydride (130 mg, 0.613 mmol) was added in one portion and the reaction mixture was stirred for a further 4 hours. The reaction mixture was partitioned between aqueous sodium carbonate (2M, 5 ml) and dichloromethane (2×1 ml) and the combined organic phases were directly applied to a silica cartridge (5 g). sequential elution with dichloromethane, dichloromethane:ethanol:ammonia, 400:8:1 then 200:8:1 then 100:8:1 gave the title product as a colourless oil.

MS (ES⁺): 495 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: split peak 2.2, 2.31 min.

### B) cis-4-(Aminomethyl)-4(3-chlorophenyl)-N-[(4-phenyl-1H-pyrazol-5-yl)methyl]cyclohexanamine hydrochloride

A mixture of the {cis-1-(3-Chloro-phenyl)-4-[(4-phenyl-2H-pyrazol-3-ylmethyl)-amino]-cyclohexylmethyl}-carbamic acid tert-butyl ester (124 mg, 0.25 mmol) trifluoroacetic acid (1 mL) and dichloromethane (1 mL) was stirred for 2 h, then blown down to dryness. The residue was purified by flash chromatography (silica, eluting sequentially with dichloromethane, dichloromethane:ethanol:ammonia, 400:8:1 then 200:8:1 then 100:8:1) to afford the free base of the title compound as a colourless oil. The oil was dissolved in methanol (1 ml) and treated with concentrated hydrochloric acid (3 drops). The mixture was concentrated in vacuo, triturated with diethyl ether and dried to give the title compound as a white solid.

MS (ES⁺): 395, 397 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 4.68 min.

### Example L2

### cis-4-(Aminomethyl)-N-[(2-benzyl-1H-imidazol-5-yl)methyl]-4-(3-chlorophenyl)cyclohexanamine hydrochloride

The title compound was prepared analogously as described in Example L1 using 2-benzyl-3H-imidazole-4-carbaldehyde instead 4-phenyl-2H-pyrazole-3-carbaldehyde.

MS (ES⁺): 409, 411 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 3.64 min.

### Example M1

### N-[cis-4-(Aminomethyl)-4-(3-chlorophenyl)cyclohexyl]-N-benzylpyridazine-3-carboxamide hydrochloride

The title compound was prepared according to Scheme M.

### A) A mixture of [cis-4-Benzylamino-1-(3-chloro-phenyl)-cyclohexylmethyl]-carbamic acid tert-butyl ester and [trans-4-benzylamino-1-(3-chloro-phenyl)-cyclohexylmethyl]-carbamic acid tert-butyl ester

Sodium triacetoxyborohydride (320 mg, 1.5 mmol) was added in one portion to a mixture of benzylamine (90 μL, 0.825 mmol), [1-(3-chlorophenyl)-4-oxo-cyclohexylmethyl]-carbamic acid tert-butyl ester (250 mg, 0.74 mmol) and acetic acid (0.5 mL) in dichloromethane (5 mL) and the reaction mixture was stirred for 18 hours. The reaction mixture was partitioned between aqueous sodium carbonate (2M, 5 ml) and dichloromethane (2×1 ml) and the organic phases were applied directly to a silica cartridge (5 g). Sequential elution with dichloromethane, dichloromethane:ethanol:ammonia, 400:8:1 then 200:8:1 then 100:8:1 gave a mixture of the title compounds in the form of a pale yellow oil.

MS (ES⁺): 429 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 2.31, 3.39 min.

### B) [cis-4-[Benzyl-(pyridazine-3-carbonyl)amino]-1-(3-chloro-phenyl)-cyclohexylmethyl]-carbamic acid tert-butyl ester and [trans-4-[benzyl-(pyridazine-3-carbonyl)amino]-1-(3-chloro-phenyl)-cyclohexylmethyl]-carbamic acid tert-butyl ester

Pyridazine-3-carboxylic acid (36 mg, 0.29 mmol) was added to a solution of the foregoing mixture of [cis-4-benzylamino-1-(3-chloro-phenyl)-cyclohexylmethyl]-carbamic acid tert-butyl ester and [trans-4-benzylamino-1-(3-chloro-phenyl)-cyclohexylmethyl]-carbamic acid tert-butyl ester (83 mg, 0.193 mmol) in dimethylformamide (3 mL) containing diisopropylethylamine (100 μL, 0.58 mmol)) and O-(7-azabenzotriazol-1-yl)-N,N,N′,N′-tetramethyluronium hexafluorophosphate (110 mg, 0.29 mmol) under a nitrogen atmosphere. After stirring at room temperature overnight, the mixture was diluted with water and extracted into ethyl acetate (2×50 ml). The combined organic phases were washed with water and brine, dried (MgSO₄) and concentrated. The residue was purified by automated flash chromatography (Silica cartridge (4 g), using gradient elution from 0%-100% ethyl acetate in cyclohexane over 15 minutes) to give the title compounds as individual diastereoisomers.

Cis diastereoisomer:

MS (ES⁺): 535, 537 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 3.95 min.

Trans diastereoisomer:

MS (ES⁺): 535, 537 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 3.84 min.

### C) N-[cis-4-(Aminomethyl)-4-(3-chlorophenyl)cyclohexyl]-N-benzylpyridazine-3-carboxamide hydrochloride

A solution of [cis-4-[benzyl-(pyridazine-3-carbonyl)amino]-1-(3-chloro-phenyl)-cyclohexylmethyl]-carbamic acid tert-butyl ester (48 mg, 0.090 mmol) in trifluoroacetic acid (0.6 mL) and dichloromethane (3 mL) was stirred at room temperature for 2 hours. The reaction mixture was applied to an SCX-2 ion exchange column and eluted sequentially with dichloromethane, methanol and a 2M solution of ammonia in methanol to the freebase of the product. The free base was further purified by flash chromatography (silica, eluting with 0-20% methanol in dichloromethane). Treatment with excess hydrogen chloride in methanol and freeze drying afforded the title compound as a beige coloured solid.

MS (ES⁺): 435, 437 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 6.14 min.

### Example M2

### N-[trans-4-(Aminomethyl)-4-(3-chlorophenyl)cyclohexyl]-N-benzylpyridazine-3-carboxamide hydrochloride

The title compound was prepared analogously as described in Example M1, step C using [trans-4-[benzyl-(pyridazine-3-carbonyl)amino]-1-(3-chloro-phenyl)-cyclohexylmethyl]-carbamic acid tert-butyl ester instead of [cis-4-[benzyl-(pyridazine-3-carbonyl)amino]-1-(3-chloro-phenyl)-cyclohexylmethyl]-carbamic acid tert-butyl ester.

MS (ES⁺): 435, 437 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 6.03 min.

### Example M3

### N-[cis-4-(Aminomethyl)-4-(3-chlorophenyl)cyclohexyl]-N-(2-phenylethyl)acetamide Hydrochloride

The title compound was prepared according to Scheme M.

### A) A mixture of [cis-1-(3-chloro-phenyl)-4-phenethylamino-cyclohexylmethyl]-carbamic acid tert-butyl ester and [trans-1-(3-chloro-phenyl)-4-phenethylamino-cyclohexylmethyl]-carbamic acid tert-butyl ester

Sodium triacetoxyborohydride (350 mg, 1.65 mmol) was added in one portion to a mixture of 2-phenyl-ethylamine (200 μL, 1.59 mmol), [1-(3-chlorophenyl)-4-oxo-cyclohexylmethyl]-carbamic acid tert-butyl ester (300 mg, 0.89 mmol) and acetic acid (0.5 mL) in dichloromethane (5 mL) and the reaction mixture was stirred for 36 hours. The reaction mixture was partitioned between sodium carbonate (2M, 5 ml) and dichloromethane (2×2 ml) and the organic phases were directly applied to a silica cartridge (10 g). Elution with dichloromethane, then dichloromethane:ethanol:ammonia, 400:8:1 then 200:8:1 then 100:8:1 gave a mixture of the products as a colourless oil.

MS (ES⁺): 443, 445 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 2.46 min.

### B) [cis-4-(Acetyl-phenethyl-amino)-1-(3-chloro-phenyl)-cyclohexylmethyl]-carbamic acid tert-butyl ester and [trans-4-(acetyl-phenethyl-amino)-1-(3-chloro-phenyl)-cyclohexylmethyl]-carbamic acid tert-butyl ester

Triethylamine (160 μL, 1.13 mmol) and acetyl chloride (40 μL, 0.57 mmol) were added to a solution of the foregoing mixture of [cis-1-(3-chlorophenyl)-4-phenethylamino-cyclohexylmethyl]-carbamic acid tert-butyl ester and [trans-1-(3-chloro-phenyl)-4-phenethylamino-cyclohexylmethyl]-carbamic acid tert-butyl ester (167 mg, 0.377 mmol) in dichloromethane (3 mL) and the reaction mixture was stirred at room temperature for 3 hours. The mixture was diluted with water and extracted with dichloromethane (2×30 mL). The combined organic phases were washed with brine, dried (MgSO4) and concentrated in vacuo. The residue was purified by flash chromatography (Silica cartridge (10 g), using gradient elution from 30%-50% ethyl acetate in cyclohexane) to give the title compounds as individual diastereoisomers.

Cis diastereoisomer:

MS (ES⁺): 485, 487 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 4.33 min.

Trans diastereoisomer:

MS (ES⁺): 485, 487 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 4.09 min.

### C) N-[cis-4-(Aminomethyl)-4-(3-chlorophenyl)cyclohexyl]-N-(2-phenylethyl)acetamide Hydrochloride

A solution of [cis-4-(acetyl-phenethyl-amino)-1-(3-chloro-phenyl)-cyclohexylmethyl]-carbamic acid tert-butyl ester (71 mg, 0.146 mmol) in trifluoroacetic acid (0.6 mL) and dichloromethane (3 mL) was stirred at room temperature for 2 hours. The reaction mixture was applied to an SCX-2 ion exchange column and eluted sequentially with dichloromethane, methanol and a 2M solution of ammonia in methanol to the freebase of the product. The free base was further purified by automated flash chromatography (silica, eluting with 0-20% methanol in dichloromethane). Treatment with excess hydrogen chloride in methanol and freeze drying afforded the title compound as a beige coloured solid.

MS (ES⁺): 385, 387 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 6.85 min.

### Example M4

### N-[trans-4-(Aminomethyl)-4-(3-chlorophenyl)cyclohexyl]-N-(2-phenylethyl)acetamide Hydrochloride

The title compound was prepared analogously as described in Example M3, step C using [trans-4-(acetyl-phenethyl-amino)-1-(3-chloro-phenyl)-cyclohexylmethyl]-carbamic acid tert-butyl ester instead of [cis-4-(acetyl-phenethyl-amino)-1-(3-chloro-phenyl)-cyclohexylmethyl]-carbamic acid tert-butyl ester.

MS (ES⁺): 385, 387 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 6.69 min.

### Example M5

### N-[cis-4-(Aminomethyl)-4-(3-chlorophenyl)cyclohexyl]-N-(2-phenylethyl)pyridazine-3-carboxamide hydrochloride

The title compound was prepared analogously as described in Example M1 using 2-phenyl-ethylamine instead of benzylamine.

MS (ES⁺): 435, 437 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 6.14 min.

### Example M6

### N-[trans-4-(Aminomethyl)-4-(3-chlorophenyl)cyclohexyl]-N-(2-phenylethyl)pyridazine-3-carboxamide hydrochloride

The title compound was prepared analogously as described in Examples M1 and M2 using 2-phenyl-ethylamine instead of benzylamine.

MS (ES⁺): 435, 437 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 6.03 min.

### Example M7

### N-[cis-4-(Aminomethyl)-4-(3-chlorophenyl)cyclohexyl]-N-(cyclopropylmethyl)pyridazine-3-carboxamide hydrochloride

The title compound was prepared analogously as described in Example M1 using C-cyclopropyl-methylamine instead of benzylamine.

MS (ES⁺): 399, 401 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 6.32 min.

### Example M8

### N-[trans-4-(Aminomethyl)-4-(3-chlorophenyl)cyclohexyl]-N-(cyclopropylmethyl)pyridazine-3-carboxamide hydrochloride

The title compound was prepared analogously as described in Examples M1 and M2 using C-cyclopropyl-methylamine instead of benzylamine.

MS (ES⁺): 399, 401 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 5.65 min.

### Example M9

### N-[cis-4-(Aminomethyl)-4-(3-chlorophenyl)cyclohexyl]-N-(2-phenoxyethyl)pyridazine-3-carboxamide hydrochloride

The title compound was prepared analogously as described in Example M1 using 2-phenoxyethylamine instead of benzylamine.

MS (ES⁺): 465, 467 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 6.71 min.

### Example M10

### N-[trans-4-(Aminomethyl)-4-(3-chlorophenyl)cyclohexyl]-N-(2-phenoxyethyl)pyridazine-3-carboxamide hydrochloride

The title compound was prepared analogously as described in Examples M1 and M2 using 2-phenoxyethylamine instead of benzylamine.

MS (ES⁺): 465, 467 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 6.49 min.

### Example M11

### N-[cis-4-(Aminomethyl)-4-(3-chlorophenyl)cyclohexyl]-N-benzylacetamide hydrochloride

The title compound was prepared analogously as described in Example M3 using benzylamine instead of 2-phenylethylamine.

MS (ES⁺): 371, 373 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 6.62 min.

### Example M12

### N-[trans-4-(Aminomethyl)-4-(3-chlorophenvncyclohexyl]-N-benzylacetamide hydrochloride

The title compound was prepared analogously as described in Examples M3 and M4 using benzylamine instead of 2-phenylethylamine.

MS (ES⁺): 371, 373 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 6.53 min.

### Example M13

### N-[cis-4-(Aminomethyl)-4-(3-chlorophenyl)cyclohexyl]-N-(cyclopropylmethyl)acetamide hydrochloride

The title compound was prepared analogously as described in Example M3 using C-cyclopropyl-methylamine instead of 2-phenylethylamine.

MS (ES⁺): 335, 337 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 6.32 min.

### Example M14

### N-[trans-4-(Aminomethyl)-4-(3-chlorophenyl)cyclohexyl]-N-(cyclopropylmethyl)acetamide hydrochloride

The title compound was prepared analogously as described in Examples M3 and M4 using C-cyclopropyl-methylamine instead of 2-phenylethylamine.

MS (ES⁺): 335, 337 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 6.14 min.

### Example M15

### N-[cis-4-(Aminomethyl)-4-(3-chlorophenyl)cyclohexyl]-N-benzylmethanesulfonamide hydrochloride and N-[trans 4-(aminomethyl)-4-(3-chlorophenyl)cyclohexyl]-N-benzylmethanesulfonamide hydrochloride

The title compounds were prepared analogously as described in Examples M3 using methane-sulphonyl chloride instead of acetyl chloride and using benzylamine instead of 2-phenylethylamine and were isolated as a mixture of diastereomers.

MS (ES⁺): 407, 409 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 6.83 min.

### Example M16

### N-[cis-4-(Aminomethyl)-4-(3-chlorophenyl)cyclohexyl]-N-(cyclopropylmethyl)methanesulfonamide hydrochloride and N-[trans-4-(aminomethyl)-4-(3-chlorophenyl)cyclohexyl]-N-(cyclopropylmethyl)methanesulfonamide hydrochloride

The title compounds were prepared analogously as described in Examples M3 using methane-sulphonyl chloride instead of acetyl chloride and using C-cyclopropyl-methylamine instead of 2-phenylethylamine and were isolated as a mixture of diastereomers.

MS (ES⁺): 371, 373 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 6.31 min.

### Example M17

### N-[cis-4-(Aminomethyl)-4-(3-chlorophenylicyclohexyl]-N-(2-Dvridyl-2-ylethyl)acetamide

The title compound was prepared analogously as described in Example M1 using 2-pyridin-2-ylethanamine instead of benzylamine.

MS (ES⁺): 386[M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 4.01 min.

### Example M18

### N-[cis-4-(Aminomethyl)-4-(3-chlorophenylicyclohexyl]-N-(3-pyridyl-2-ylethyl)acetamide

The title compound was prepared analogously as described in Example M1 using 3-pyridin-2-ylethanamine instead of benzylamine.

MS (ES⁺): 386[M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 4.51 min.

### Example N1

### N-[cis-4-(Aminomethyl)-4-(3-chlorophenyl)cyclohexyl]-N-(2-phenylethyl)pyridazine-4-carboxamide hydrochloride

The title compound was prepared by the route shown in Scheme N.

### A) N-[8-(3-Chloro-phenyl)-1,4-dioxa-spiro[4.5]dec-8-ylmethy]-2,2,2-trifluoroacetamide

Trifluoroacetic anhydride (5.5 mL, 39.57 mmol) was added at 0° C. to a stirred solution of C-[8-(3-chlorophenyl)-1,4-dioxa-spiro[4.5]dec-8-yl]-methylamine (7.42, 26.33 mmol) and diisopropylethylamine (18.4 mL, 105.64 mmol) in tetrahydrofuran (200 mL) and the resulting mixture stirred overnight, warming to room temperature. The mixture was diluted with ethyl acetate (100 mL) and 0.5N hydrochloric acid (100 mL). The aqueous layer was separated and extracted with EtOAc (100 mL×2). The combined organic phases were washed with saturated aqueous sodium bicarbonate (100 mL), brine (100 mL), dried (MgSO4) and evaporated to give the title compound as an orange coloured gum which was used directly in the next step.

### B) N-[1-(3-Chloro-phenyl)-4-oxo-cyclohexylmethyl]-2,2,2-trifluoroacetamide

The foregoing product, N-[8-(3-chloro-phenyl)-1,4-dioxa-spiro[4.5]dec-8-ylmethyl]-2,2,2-trifluoroacetamide (10.4 g, 26.3 mmol), was dissolved in a mixture of acetic acid (100 mL) and water (20 mL) and the solution stirred at ambient temperature for 68 hours. The mixture was diluted with ethyl acetate (300 mL) and washed with water (3×100 mL), and saturated aqueous sodium bicarbonate (100 mL) and the pH was adjusted to 9 by addition of 10N sodium hydroxide. The organic layer was collected, washed with brine (50 mL), dried (MgSO4) and evaporated to give a dark orange oil that solidified on standing. The gum was purified by automated flash chromatography (Silica (330 g cartridge), gradient elution with 5-40% ethyl acetate in cyclohexane). Appropriate fractions were combined and evaporated to give the title compound as an off-white solid.

MS (ES⁻): 332, 334 [M−H]⁻.

T_{R} [HPLC, Phenomenex Luna 3 micron C18; 5-95% CH₃CN+0.1% Formic acid/H₂O+0.1% Formic acid for 5 min, flow 2.0 ml/min]: 3.13 min.

### C) A mixture of N-[cis-1-(3-chloro-phenyl)-4-phenethylamino-cyclohexylmethyl]-2,2,2-trifluoroacetamide and N-[trans-1-(3-chloro-phenyl)-4-Dhenethylamino-cyclohexylmethyl]-2,2,2-trifluoroacetamide

Sodium triacetoxyborohydride (216 mg, 1.01 mmol) and acetic acid (58 μL, 1.01 mmol) were added to a solution of N-[1-(3-chloro-phenyl)-4-oxo-cyclohexylmethyl]-2,2,2-trifluoroacetamide (170 mg, 0.50 mmol) and 2-phenylethylamine (96 μL, 0.76 mmol) in 1,2-dichloroethane (2.5 mL) and the mixture was stirred at room temperature overnight. The reaction was quenched with saturated aqueous sodium bicarbonate and extracted with dichloromethane. The organic phase was washed with saturated aqueous sodium bicarbonate and water, filtered through a phase separator and concentrated in vacuo. The residue was purified by automated flash chromatography (silica (4 g), gradient elution with dichloromethane to dichloromethane:methanol 93:7) to give a mixture of the title compounds as a yellow oil.

MS (ES⁻): 439, 441 [M−H]⁻.

T_{R} [HPLC, Phenomenex Luna 3 micron C18; 5-95% CH₃CN+0.1% Formic acid/H₂O+0.1% Formic acid for 5 min, flow 2.0 ml/min]: 2.58, 2.68 min.

### D) Pyridazine-4-carboxylic acid {cis-4-(3-chloro-phenyl)-4-[(2,2,2-trifluoro-acetylamino)-methyl]-cyclohexyl}-phenethyl-amide and pyridazine-4-carboxylic acid {trans-4-(3-chloro-phenyl)-4-[(2,2,2-trifluoro-acetylamino)-methyl]-cyclohexyl}-phenethyl-amide

Diisopropylethylamine (134 μL, 0.78 mmol) and O-(7-azabenzotriazol-1-yl)-N,N,N′,N′-tetramethyluronium hexafluorophosphate (182 mg, 0.47 mmol) were added to a solution of pyridazine-4-carboxylic acid (59.4 mg, 0.47 mmol) and a mixture of N-[cis-1-(3-chloro-phenyl)-4-phenethylamino-cyclohexylmethyl]-2,2,2-trifluoroacetamide and N-[trans-1-(3-chloro-phenyl)-4-phenethylamino-cyclohexylmethyl]-2,2,2-trifluoroacetamide (191 mg, 0.43 mmol) in dimethylformamide (3.5 mL). The mixture was stirred at room temperature for 72 hours, and was then quenched by the addition of saturated aqueous sodium bicarbonate and extracted with dichloromethane. The organic phase was washed with water (3 times), filtered through a phase separator and concentrated in vacuo. The residue was purified by automated flash chromatography (Silica (12 g), gradient elution from neat cyclohexane to 100% ethyl acetate). This gave the title compounds as individual diastereoisomers.

Cis diastereoisomer

MS (ES⁺): 545, 547 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 3.77 min.

Trans diastereoisomer:

MS (ES⁺): 545 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 3.73 min.

### E) N-[cis-4-(Aminomethyl)-4-(3-chlorophenyl)cyclohexyl]-N-(2-phenylethyl)pyridazine-4-carboxamide hydrochloride

A solution of potassium carbonate (107.6 mg, 0.779 mmol) in water (1.5 mL) was added to a solution of pyridazine-4-carboxylic acid {cis-4-(3-chloro-phenyl)-4-[(2,2,2-trifluoro-acetylamino)-methyl]-cyclohexyl}-phenethyl-amide (85 mg, 0.155 mmol) in methanol (1.5 mL) and the mixture was stirred for 60° C. for 2 hours. After diluting with ethyl acetate, the organic phase was separated, washed with water, dried (Na₂SO₄) and concentrated in vacuo. The residue was purified by ion exchange chromatography (SCX-2 column, eluting with dichloromethane/methanol 1:1 then 1.25M ammonia in methanol) to afford the freebase of the title compound which was converted to the hydrochloride salt by treatment with 1.25M hydrogen chloride in methanol and drying in vacuo.

MS (ES⁺): 449 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 6.73 min.

### Example N2

### N-[cis-4-(Aminomethyl)-4-(3-chlorophenyl)cyclohexyl]-N-(cyclopropylmethyl)pyridazine-4-carboxamide hydrochloride

The title compound was prepared analogously as described in Examples N1 using C-cyclopropyl-methylamine instead of 2-phenylethylamine.

MS (ES⁺): 399, 401 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 6.05 min.

### Example N3

### N-[cis-4-(Aminomethyl)-4-(3-chlorophenyl)cyclohexyl]-N-methylpyridazine-4-carboxamide hydrochloride

The title compound was prepared analogously as described in Examples N1 using methylamine instead of 2-phenylethylamine.

MS (ES⁺): 359 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 5.05 min.

### Example N4

### N-[cis-4-(Aminomethyl)-4-(3-chlorophenyl)cyclohexyl]-N-methylpyridazine-3-carboxamide hydrochloride

The title compound was prepared analogously as described in Examples N1 using methylamine instead of 2-phenylethylamine and pyridazine-3-carboxylic acid instead of pyridazine-4-carboxylic acid.

MS (ES⁺): 359 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 5.06 min.

### Example N5

### N-[cis-4-(Aminomethyl)-4-(3-chlorophenyl)cyclohexyl]-N-(4-Pyridyl-2-ylethyl)acetamide

The title compound was prepared analogously as described in Example N1 using 4-pyridin-2-ylethanamine instead of 2-phenylethylamine and using acetyl chloride instead of pyridazine-4-carboxylic acid.

MS (ES⁺): 386 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 4.36 min.

### Example O1

### N-[cis-4-(Aminomethyl)-4-(3-chlorophenyl)cyclohexyl]-3-(trifluoromethyl)[1,2,3]triazolo[4,3-b]pyridazine-6-amine hydrochloride

The title compound was prepared according to Scheme O.

### A) tert-Butyl{[cis-1-(3-chlorophenol)-4-{[3-trifluoromethyl)[1,2,4]triazolo[4,3-b]pyridazine-6-yl]amino}-cyclohexyl]methyl}carbamate

A solution of [cis-4-amino-1-(3-chlorophenyl)-cyclohexylmethyl]-carbamic acid tert butyl ester [Example E1] (50 mg, 0.147 mmol), 6-chloro-3-(trifluoromethyl)[1,2,3]triazolo[4,3-b]pyridazine (34.5 mg, 0.15 mmol) and DIPEA in DMA was heated under microwave irradiation in a Smith Synthesiser at 130° C. for 45 min. The reaction mixture was diluted with EtOAc (100 mL) and washed successively with water (2×10 mL) and brine (10 mL), then dried (Na₂SO₄) and concentrated in vacuo. Purification by a silica-gel cartridge, eluting from DCM to DCM/MeOH afforded the title compound.

MS (ES⁺): 525[M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 4.18 min.

### B) N-[cis-4-(Aminomethyl)-4-(3-chlorophenyl)cyclohexyl]-3-(trifluoromethyl)[1,2,3]triazolo[4,3-b]pyridazine-6-amine hydrochloride

A solution of tert-butyl{[cis-1-(3-chlorophenyl)-4-{[3-trifluoromethyl)[1,2,4]triazolo[4,3-b]pyridazine-6-yl]amino}-cyclohexyl]methyl}carbamate (54 mg, 0.102 mmol) in trifluoroacetic acid (2 mL) and dichloromethane (3 mL) was stirred at room temperature for 0.5 hours. The reaction mixture was concentrated in vacuo and the residue was purified by ion exchange chromatography (SCX-2 column, eluting with dichloromethane/methanol 1:1 then 1.25M ammonia in methanol) to afford the freebase of the title compound, which was converted to the hydrochloride salt by treatment with 1.25M hydrogen chloride in methanol and drying in vacuo.

MS (ES⁺): 425 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 6.88 min.

### Example P1

### 1-[cis-4-(Aminomethyl)-4-(3-chlorophenyl)cyclohexyl]piperidine-2-one

The title compound was prepared according to Scheme P.

### A) tert-Butyl({cis-4-[(5-chloropentanoyl)amino]1-1(3-chlorophenyl)cyclohexyl}methyl) carbamate

A rapidly stirred mixture of [cis-4-amino-1-(3-chlorophenyl)-cyclohexylmethyl]-carbamic acid tert butyl ester (300 mg, 0.88 mmol) in chloroform (3 mL) was treated with saturated aqueous sodium carbonate (2.5 mL) then 5-chlorovaleryl chloride (143 mg, 0.88 mmol). After 0.75 hours the reaction mixture was poured onto a hydrophobic phase separater and the aqueous layer further washed with DCM (3×5 mL). The combined organic extracts were concentrated in vacuo to afford the title compound.

MS (ES⁺): 458[M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 3.91 min.

### B) tert-Butyl{[cis-1-(3-chlorophenyl)-4-(2-oxopiperidin-1-yl)cyclohexyl]methyl}carbamate

As solution of tert-butyl({cis-4-[(5-chloropentanoyl)amino]1-1(3-chlorophenyl)cyclohexyl}methyl)carbamate (211 mg, 0.440 mmol) in DMF (1 mL) was added drop-wise to a solution in DMF (0.5 mL) of sodium hydride (1.4 eq, 26 mg, 0.618 mmol). The reaction mixture was stirred for 18 hours then quenched via addition of water (20 mL) and extracted into ethyl acetate (2×20 mL). The combined organic extracts were washed further with water (10 mL) and brine (10 mL) before drying (Na₂SO₄), filtering and concentrating in vacuo. The residue was purified by flash silica-gel cartridge, eluting with ethyl acetate/cyclohexane (2:3) to give the title compound.

MS (ES⁺): 421[M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 3.70 min.

### C) 1-[cis-4-(Aminomethyl)-4-(3-chlorophenyl)cyclohexyl]piperidine-2-one blpyridazine-6-amine hydrochloride

A solution of tert-butyl{[cis-1-(3-chlorophenyl)-4-(2-oxopiperidin-1-yl)cyclohexyl]methyl}carbamate (142 mg, 0.337 mmol) in trifluoroacetic acid (2 mL) and dichloromethane (3 mL) was stirred at room temperature for 1 hour. The reaction mixture was concentrated in vacuo and the residue was purified by ion exchange chromatography (SCX-2 column, eluting with dichloromethane/methanol 1:1 then 1.25M ammonia in methanol) to afford the freebase of the title compound, which was converted to the hydrochloride salt by treatment with 1.25M hydrogen chloride in methanol and drying in vacuo.

MS (ES⁺): 321 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 5.06 min.

### Example P2

### 1-[cis-1-[3-Chlorophenyl)-4-piperidin-1-ylcyclohexyl]methanamine

The title compound was prepared according to Scheme P.

A solution of 1-[cis-4-(aminomethyl)-4-(3-chlorophenyl)cyclohexyl]piperidine-2-one b]pyridazine-6-amine hydrochloride (50 mg, 0.155 mmol) in THF (1.5 mL) was treated drop-wise with borane (1M in THF) and then heated to reflux for 18 hours. A further quantity of borane (3 eq, 0.47 mL, 0.465 mmol) was added and refluxing continued for 24 hours. The crude reaction mixture was concentrated in vacuo, then re-dissolved in MeOH (1 mL) and treated with aqueous 1N HCl (1 mL) prior to refluxing for 7 hours. The reaction mixture was cooled and partitioned between aqueous 3N NaOH (50 mL) and ethyl acetate (3×75 mL). The combined organic extracts were dried (Na₂SO₄), filtered and concentrated in vacuo. The residue was purified by flash silica-gel chromatography, eluting with DCM/MeOH (1:1) to give the free-base of the title compound. The addition of 1.25N HCl in MeOH and concentration in vacuo afforded the title compound.

MS (ES⁺): 307 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 1.08 & 3.63 min.

### Example Q1

### Pyridazine-3-carboxylic acid [4-aminomethyl-4-(3-chloro-phenyl)-1-methyl-cyclohexyl]-amide

### A) [8-(3-Chloro-phenyl)-1,4-dioxa-spiro[4.5]dec-8-ylmethyl]-carbamic acid benzyl ester

A solution of C-[8-(3-chlorophenyl)-1,4-dioxa-spiro[4.5]dec-8-yl]-methylamine (9.9 g, 35.13 mmol), N-(benzyloxycarbonyloxy)succinimide (9.65 g, 38.72 mmol) and DIPEA (12.25 mL, 70.33 mmol) in DMF (25 mL) is stirred at rt during 4 h before evaporation of the solvent. The residue is dissolved with ethyl acetate and an aqueous 1N HCl solution, the aqueous phase is extracted with ethyl acetate, the combined organic phases are washed with water, dried and evaporated to give the title compound.

MS (ES⁺): 416-418[M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 3.94 min.

### B) [1-(3-Chloro-phenyl)-4-oxo-cyclohexylmethyl]-carbamic acid benzyl ester

The title compound was prepared analogously as described in Example N1 step B using 8-(3-Chloro-phenyl)-1,4-dioxa-spiro[4.5]dec-8-ylmethyl]-carbamic acid benzyl ester instead of N-[8-(3-chloro-phenyl)-1,4-dioxa-spiro[4.5]dec-8-ylmethyl]-2,2,2-trifluoroacetamide.

MS (ES⁺): 372-374 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 3.63 min.

### C) [1-(3-Chloro-phenyl)-4-(2-methyl-propane-2-sulfinylimino)-cyclohexylmethyl]-carbamic acid benzyl ester

To 1-(3-Chloro-phenyl)-4-oxo-cyclohexylmethylpcarbamic acid benzyl ester (5.58 g, 15.01 mmol) and 2-methyl-2-propanesulfinamide (2 g, 16.5 mmol) in THF (60 mL) is added titanium tetraethoxide (6.3 mL, 30.05 mmoL) before stirring at 70-75° C. during 40 h. The mixture is poured in Rochelle's salt, filtered and extracted with ethyl acetate. The combined organic phases are washed with brine, dried and evaporated before purification by flash chromatography on silica gel (cyclohexane/Ethyl acetate 8/2 to 4/6) to give the title compound.

MS (ES⁺): 475 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 3.34 min.

### D) [1-(3-Chloro-phenyl)-4-methyl-4-(2-methyl-propane-2-sulfinylamino)-cyclohexylmethyl]-carbamic acid benzyl ester

To [1-(3-Chloro-phenyl)-4-(2-methyl-propane-2-sulfinylimino)-cyclohexylmethyl]-carbamic acid benzyl ester (300 mg, 0.63 mmol) in DCM (6 mL) is added at 0° C. a 3M methylmagnesium bromide solution in ether (0.842 mL, 2.52 mmol) before stirring at rt over night. The reaction is quenched with an aqueous saturated NH4Cl solution, extracted with DCM and the combined organic phases are dried and evaporated before purification by flash chromatography on silica gel (cyclohexane/Ethyl acetate 9/1 to 25/75) to give the title compound.

MS (ES⁺): 491 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 3.56 min.

### E) [4-Amino-1-(3-chloro-phenyl)-4-methyl-cyclohexylmethyl]-carbamic acid benzyl ester

To [1-(3-Chloro-phenyl)-4-methyl-4-(2-methyl-propane-2-sulfinylamino)-cyclohexylmethyl]-carbamic acid benzyl ester (145 mg, 0.295 mmol) in dioxane/methanol (0.472 mL/1 mL) is added a 1.25M HCl solution in methanol (0.472 mL, 0.59 mmol) before stirring at rt during 3 h. The solvent is evaporated before purification onto a SCX-2 cartridge (DCM/MeOH 1/1 and 2N NH3 in MeOH) to give the title compound.

TLC (DCM/2N NH3 in MeOH 94/6): 0.16.

### F) 1-(3-Chloro-phenyl)-4-methyl-4-[(pyridazine-3-carbonyl)-amino]-cyclohexylmethyl}-carbamic acid benzyl ester

The title compound was prepared analogously as described in Example E1 step H using [4-Amino-1-(3-chloro-phenyl)-4-methyl-cyclohexylmethyl]-carbamic acid benzyl ester instead of [cis-4-Amino-1-(3-chlorophenyl)-cyclohexylmethyl]-carbamic acid tert butyl ester.

Isomer 1: MS (ES⁺): 493 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.40 min.

Isomer 2: MS (ES⁺): 493 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.60 min.

### G) Pyridazine-3-carboxylic acid [4-aminomethyl-4-(3-chloro-phenyl)-1-methyl-cyclohexyl]-amide

The title compound was prepared analogously as described in Example D60 using 1-(3-Chloro-phenyl)-4-methyl-4-[(pyridazine-3-carbonyl)-amino]-cyclohexylmethyl)-carbamic acid benzyl ester instead of 4-[4-Aminomethyl-4-(3-chloro-phenyl)-cyclohexyl]-piperazine-1-carboxylic acid benzyl ester.

Isomer 1: MS (ES⁺): 359 μM⁺

T_{R} [HPLC, Higgins Clipeus 5 micron C18; 5-95% CH₃CN+0.1% Formic acid/H₂O+0.1% Formic acid for 20 min, flow 2.0 ml/min]: min.

Isomer 2: MS (ES⁺): 359 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: min.

### Example Q2

### Pyridazine-4-carboxylic acid [4-aminomethyl-4-(3-chloro-phenyl)-1-methyl-cyclohexyl]-amide

The title compound was prepared analogously as described in Example Q1 using pyridazine-3-carboxylic acid instead of pyridazine-2-carboxylic acid.

Isomer 1: MS (ES⁺): 359 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: min.

Isomer 2: MS (ES⁺): 359 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: min.

### Example R1

### C-[1-(3-Chloro-phenyl)-4-phenyl-cyclohex-3-enyl]-methylamine

### A) Trifluoro-methanesulfonic acid 4-aminomethyl-4-(3-chloro-phenyl)-cyclohex-1-enyl ester

To [1-(3-Chlorophenyl)-4-oxo-cyclohexylmethyl]-carbamic acid tert-butyl ester (507 mg, 1.5 mmol) in THF (15 mL) is added at −78° C. a 1.8M LDA solution in THF/hexane (1.77 mL, 3.18 mmol). After stirring at this temperature during 1 h, N-phenyl-bis(trifluoromethanesulfonimide (810 mg, 2.267 mmol) in THF (6 mL) is added before warming slowly at rt and stirring overnight. The mixture is poured into water, extracted with ethyl acetate, the combined organic phases are washed with brine, dried and evaporated to give the title compound.

### B) C-[1-(3-Chloro-phenyl)-4-phenyl-cyclohex-3-enyl]-methylamine

To a mixture of -methanesulfonic acid 4-aminomethyl-4-(3-chloro-phenyl)-cyclohex-1-enyl ester (340 mg, 0.724 mmol), phenylboronic acid (140 mg, 1.15 mmol) and an aqueous 1N Na2CO3 solution (4.4 mL, 4.4 mmol) in DME (10 mL) is added tetrakis(triphenylphosphine)palladium (84 mg, 0.078 mmol) before stirring at 80° C. during 16 h. The reaction is quenched with water, extracted with ethyl acetate, the combined organic phases are washed with an aqueous saturated NaHCO3 solution, dried and evaporated to give a crude compound. The protected amine is dissolved with DCM (5 mL) and TFA (0.5 mL) before stirring at rt during 16 h. The solvent are evaporated before purification by flash chromatography on silica gel (DCM/ethylacetate/methanol 78/20/2) to give the title compound.

MS (ES⁺): 298-300 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 2.46 min.

### Example R2

### C-[1-(3-Chloro-phenyl)-4-pyridin-3-yl-cyclohex-3-enyl]-methylamine

The title compound was prepared analogously as described in Example R1 using 3-pyridineboronic acid instead of phenylboronic acid.

MS (ES⁺): 299 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 4.23 min.

### Example S1

### C-[1-(3-Chloro-benzyl)-4-(3-trifluoromethyl-5,6-dihydro-8H-[1,2,4]triazolo[4,3-a]pyrazin-7-yl)-cyclohexyl]-methylamine

### A) 8-(3-Chloro-benzyl)-1,4-dioxa-spiro[4.5]decane-8-carbonitrile

To a solution of diisopropylamine (1.21 mL, 8.66 mmol) in THF (50 mL) is added at −78° C. a 1.6M BuLi solution in hexane (4.94 mL, 7.91 mmol) be fore stirring at −78° C. during 15 min. A solution of 1,4-Dioxa-spiro[4.5]decane-8-carbonitrile (1.26 g, 7.53 mmol) in THF (25 mL) is added, the mixture is stirred at −78° C. during 1 h before addition of 3-chlorobenzylbromide (1.09 mL, 8.29 mmol) and warming to rt for a stirring during 3 h. The reaction is quenched with water, extracted with Et2O, the combined organic phases are washed with H2O, dried and evaporated before purification by flash chromatography on silica gel (cyclohexane/ethylacetate 1/0 to 85/15) to give the title compound.

MS (ES⁺): 291 [M+H⁺.

T_{R} [HPLC, Phenomenex Luna 3 micron C18; 5-95% CH₃CN+0.1% Formic acid/H₂O+0.1% Formic acid for 5 min, flow 2.0 ml/min]: 3.7 min.

### B) 1-(3-Chloro-benzyl)-4-oxo-cyclohexanecarbonitrile

The title compound was prepared analogously as described in Example D1 step G using 8-(3-Chloro-benzyl)-1,4-dioxa-spiro[4.5]decane-8-carbonitrile instead of [8-(3-chlorophenyl)-1,4-dioxa-spiro[4.5]dec-8-ylmethyl]-carbamic acid tert-butyl ester.

MS (ES⁺): 246 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.33 min.

### C) 1-(3-Chloro-benzyl)-4-(3-trifluoromethyl-5,6-dihydro-8H-[1,2,4]triazolo[4,3-a]pyrazin-7-yl)-cyclohexanecarbonitrile

The title compound was prepared analogously as described in Example C1 step A using 1-(3-Chloro-benzyl)-4-oxo-cyclohexanecarbonitrile instead of 4-oxo-1-phenyl-cyclohexanecarbonitrile.

MS (ES⁺): 465 [M+CH₃CN+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.43 min.

### D) C-[1-(3-Chloro-benzyl)-4-(3-trifluoromethyl-5,6-dihydro-8H-[1,2,4]triazolo[4,3-a]pyrazin-7-yl)-cyclohexyl]-methylamine

The title compound was prepared analogously as described in Example H1 step E using 1-(3-Chloro-benzyl)-4-(3-trifluoromethyl-5,6-dihydro-8H-[1,2,4]triazolo[4,3-a]pyrazin-7-yl)-cyclohexanecarbonitrile instead of cis-4-(3-Chlorophenyl)-cyano-cyclohexanecarboxylic acid 2-trifluoromethyl-benzylamide.

MS (ES⁺): 428 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.52 min.

### Example T1

### C-[(1S,3R)-1-m-Tolyl-3-(3-trifluoromethyl-5,6-dihydro-8H-[1,2,4]triazolo[4,3-a]pyrazin-7-yl)-cyclohexyl]-methylamine

### A) 3-m-Tolyl-cyclohex-2-enone

To a 1M m-toluenemagnesium bromide solution in THF (8.56 mL) is added at 0° C. 3-Ethoxy-cyclohex-2-enone (1 g, 7.13 mmol) in THF (1 mL) before stirring at rt during 1 h. The reaction is quenched with an aqueous saturated NH4Cl solution, extracted with DCM, the organic phase is dried and evaporated before purification by flash chromatography on silica gel (cyclohexane/ethylacetate 9/1 to 2/1) to give the title compound.

MS (ES⁺): 187 μM⁺

HPLC (Zorbax SB C18, 2 min method (0-0.8 min 10-95% ACN, 0.8-1.5 min 95% ACN, 1.5-1.6 min 95-10% ACN, 1.6-2 min 10% ACN): 1.367 min.

### B) 3-Oxo-1-m-tolyl-cyclohexanecarbonitrile

To 3-m-Tolyl-cyclohex-2-enone (550 mg, 2.95 mmol) in DMF/H2O (10 mL, 1.75 mL) are added KCN (385 mg, 5.9 mmol) and trimethylamine hydrochloride (425 mg, 4:42 mmol) before stirring at 95° C. during 6 h. The reaction is quenched with an aqueous saturated NaHCO3 solution, extracted with ethyl acetate, the organic phase is dried and evaporated before purification by flash chromatography on silica gel (cyclohexane/ethylacetate 9/1 to 1/1) to give the title compound.

MS (ES⁺): 214 [M+H]⁺

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.6 min.

### C) C-[(1S,3R)-1-m-Tolyl-3-(3-trifluoromethyl-5,6-dihydro-8H-[1,2,4]triazolo[4,3-a]pyrazin-7-yl)-cyclohexyl]-methylamine

The title compound was prepared analogously as described in Example S1 step C-D using 3-Oxo-1-m-tolyl-cyclohexanecarbonitrile instead of 1-(3-Chloro-benzyl)-4-oxo-cyclohexane carbonitrile.

MS (ES⁺): 394 [Mi-H]⁺

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.608 min.

### Example U1

### 1-[trans-1-Aminomethyl-4-(3-trifluoromethyl-5,6-dihydro-8H-[1,2,4]triazolo[4,3-a]pyrazin-7-yl)-cyclohexyl]-4-methyl-pyrrolidin-2-one dihydrochloride

The title compound was prepared according to Scheme U.

### A) 8-Nitromethyl-1,4-dioxa-spiro[4.5]decan-8-ol

A mixture of Cyclohexanedione monoethylene acetal (1.0 g, 6.4 mmol), 1,4-Diazabicyclo[2.2.2]octane (730 mg, 6.31 mmol), Lithium bromide (270 mg, 3.12 mmol) and Nitromethane (0.86 ml, 14.8 mmol) was immediately melted by heating at 100° C. The resulting solution was stirred at 100° C. for 20 minutes. The reaction mixture was partitioned between water and dichloromethane. The organic phase was washed with brine, dried over Sodium sulfate and concentrated in vacuo. The residue was purified by silica gel chromatography using gradient elution from 100% dichloromethane to dichloromethane/methanol 96:4. Fractions containing the product were concentrated in vacuo. The residue was purified by silica gel chromatography using gradient elution from 100% cyclohexane to cyclohexane/ethylacetate 1:1. Fractions containing the product were concentrated in vacuo to give the title compound as an amorphous white solid.

MS (ES⁺): 218 [M+H]⁺.

### B) 8-Nitromethylene-1,4-dioxa-spiro[4.5]decane

To a solution of 8-Nitromethyl-1,4-dioxa-spiro[4.5]decan-8-ol (7.94 g, 36.6 mmol) in dichloromethane (100 ml) was added Triethylamine (12.7 ml, 91.4 mmol) at −40° C. The resulting mixture was stirred at −40° C. for 5 minutes, then Methane sulfonyl chloride (4.27 ml, 54.8 mmol) was added dropwise. The resulting mixture was stirred at −40° C. for 2 h, then again Triethylamine (12.7 ml, 91.4 mmol) and Methane sulfonyl chloride (4.27 ml, 54.8 mmol) were added dropwise at −40° C. The resulting mixture was stirred at −40° C. for 1 h. The reaction mixture was diluted with dichloromethane then the organic phase was washed sequentially with 1N Hydrochloric acid, water and brine, dried over Sodium sulfate and concentrated in vacuo. The residue was purified by silica gel chromatography using gradient elution from cyclohexane/ethylacetate 4:1 to cyclohexane/ethylacetate 1:1. Fractions containing the product were concentrated in vacuo to give the title compound as a pale yellow oil.

MS (ES⁺): 201 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.35 min.

### C) 4-Methyl-1-(8-nitromethyl-1,4-dioxa-spiro[4.5]dec-8-yl)-pwrolidin-2-one

4-Methyl-pyrrolidin-2-one (268 mg, 2.70 mmol), Potassium tert-butoxide (303 mg, 2.70 mmol) and 18-Crown-6 (715 mg, 2.70 mmol) were dissolved in tetrahydrofurane (22 ml) at 0° C. The resulting solution was stirred at 0° C. for 1 h, then 8-Nitromethylene-1,4-dioxa-spiro[4.5]decane (539 mg, 2.70 mmol) was added at −78° C. The mixture was allowed to warm up to room temperature over 2 h of stirring. The reaction mixture was quenched with saturated aqueous Ammonium chloride solution and extracted 3× into ethyl acetate. The combined organic phases were washed with brine, dried over Sodium sulfate and concentrated in vacuo. The residue was purified by silica gel chromatography using elution with cyclohexane/ethylacetate 1:1. Fractions containing the product were concentrated in vacuo to give the title compound as a colourless oil.

MS (ES⁺): 299 [M+H]⁺.

### D) 4-Methyl-1-(1-nitromethyl-4-oxo-cyclohexv1)-pyrrolidin-2-one

To a solution of 4-Methyl-1-(8-nitromethyl-1,4-dioxa-spiro[4.5]dec-8-yl)-pyrrolidin-2-one (489 mg, 1.51 mmol) in acetic acid (10 ml) was added water (3 ml). The resulting mixture was stirred at room temperature for 60 h, then at 50° C. for 5 h and finally at 60° C. for 4.5 h. The mixture was diluted with ethylacetate and washed 3× with water. The aqueous phase was extracted with ethyl acetate. The combined organic phases were washed sequentially with 1N Sodium hydroxide solution, water and brine. The product remains in aqueous phase. All the combined aqueous phases were neutralized with 1N Hydrochloric acid and concentrated in vacuo. The residue was taken up in Acetonitrile, the suspension was filtered and the filtrate was concentrated in vacuo to give the title compound as crystalline needles.

MS (ES⁺): 255 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.84 min.

### E) 4-Methyl-1-[1-nitromethyl-4-(3-trifluoromethyl-5,6-dihydro-8H-[1,2,4]triazolo[4,3-a]pyrazin-7-yl)-cis-cyclohexyl]-pyrrolidin-2-one formate and 4-Methyl-1-[1-nitromethyl-4-(3-trifluoromethyl-5,6-dihydro-8H-[1,2,4]triazolo[4,3-a]pyrazin-7-yl)-trans-cyclohexyl]-pyrrolidin-2-one formate

To a solution of 4-Methyl-1-(1-nitromethyl-4-oxo-cyclohexyl)-pyrrolidin-2-one (95 mg, 0.374 mmol) in 1,2-Dichloroethane (6 ml) was added 3-Trifluoromethyl-5,6,7,8-tetrahydro-[1,2,4]triazolo[4,3-a]pyrazine (135 mg, 0.591 mmol), N,N-Diisopropylethylamine (0.1 ml, 0.584 mmol) and acetic acid (20 μl, 0.393 mmol). The resulting mixture was stirred at room temperature for 30 minutes, then Sodium triacetoxyborohydride (167 mg, 0.788 mmol) was added. The resulting mixture was stirred at room temperature for 6 h. The mixture was quenched with water, then concentrated in vacuo to give the title compound as a mixture of diastereomeric isomers, which were separated and purified by prep. HPLC (Waters. SunFire Prep C18 ODB 5 μm 19×50 mm, flow 20 ml/min, 15 min method (0-2.5 min 20% ACN, 2.5-17.5 min 20-50% ACN, 17.5-20.0 min 50% ACN). Fractions containing the products were lyophilized individually to give the individual title compounds as white solids as formic acid salts.

MS (ES⁺): 431 [M+H]⁺ (trans) and MS (ES⁺): 431 [M+H]⁺ (cis)

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.00 min (trans) and 3.11 min (cis).

### F) 1-[trans-1-Aminomethyl-4-(3-trifluoromethyl-5,6-dihydro-8H-[1,2,4]triazolo[4,3-a]pyrazin-7-yl)-cyclohexyl]-4-methyl-pyrrolidin-2-one dihydrochloride

4-Methyl-1-[1-nitromethyl-4-(3-trifluoromethyl-5,6-dihydro-8H-[1,2,4]triazolo[4,3-a]pyrazin-7-yl)-trans-cyclohexyl]-pyrrolidin-2-one formate (15 mg, 0.031 mmol) was dissolved in 4N Hydrochloric acid (2 ml) and lyophilized. The obtained 4-Methyl-1-[1-nitromethyl-4-(3-trifluoromethyl-5,6-dihydro-8H-[1,2,4]triazolo[4,3-a]pyrazin-7-yl)-trans-cyclohexyl]-pyrrolidin-2-one hydrochloride was dissolved 1N Hydrochloric acid (4 ml), then 10% Palladium on charcoal (10 mg, 0.009 mmol) was added. The resulting mixture was stirred at room temperature for 16 h under hydrogen atmosphere. The mixture was filtered and the filtrate was lyophilized to give the title compound as a beige solid.

MS (ES⁺): 401 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.62 min.

### Example U2

### 1-[cis-1-Aminomethyl-4-(3-trifluoromethyl-5,6-dihydro-8H-[1,2,4]triazolo[4,3-a]pyrazin-7-yl)-cyclohexyl]-4-methyl-pyrrolidin-2-one dihydrochloride

The title compound was prepared analogously as described in Example U1, step F from 4-Methyl-1-[1-nitromethyl-4-(3-trifluoromethyl-5,6-dihydro-8H-[1,2,4]triazolo[4,3-a]pyrazin-7-yl)-trans-cyclohexyl]-pyrrolidin-2-one formate.

MS (ES⁺): 401 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.49 min.

### Example V1

### C-[1-m-Tolyl-3-(3-trifluoromethyl-5,6-dihydro-8H-[1,2,4]triazolo[4,3-a]pyrazin-7-yl)-cyclopentyl]-methylamine hydrochloride

The title compound was prepared according to Scheme V.

### A) 2-Allyl-2-m-tolyl-pent-4-enenitrile

To a mixture of 3-Methylbenzylcyanide (5 g, 37.4 mmol) and Hexadecyltributylphosphonium bromide (391 mg, 0.747 mmol) in 50% aqueous Sodium hydroxide solution (15 ml) was added slowly retaining temperature below 50° C. Allyl bromide (8.3 ml, 86 mmol). When the addition was complete, the reacti mixture was stirred at room temperature for 24 h. The reaction mixture was extracted into toluene. T combined organic phases were dried and concentrated in vacuo to give the title compound as a whi solid.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 4.20 min.

### B) 1-m-Tolyl-cyclopent-3-enecarbonitrile

2-Allyl-2-m-tolyl-pent-4-enenitrile (3.0 g, 13.9 mmol) was dissolved in dichloromethane (300 ml) under nitrogen atmosphere. The resulting mixture was heated to 40° C., then (1,3-Bis(2,4,6-trimethylphenyl)-2-imidazolidinylidene)dichloro(phenylmethylene)-(tricyclohexylphosphine)ruthenium (Grubbs Catalyst, 2nd Generation) (1.15 g, 1.39 mmol) was added. The resulting mixture was stirred at 40° C. for 16 h. The reaction mixture was concentrated in vacuo. The residue was purified by flash chromatography (Silica cartridge) using gradient elution from 100% cyclohexane to cyclohexane/ethylacetate 1:1. Fractions containing the product were concentrated in vacuo to give the title compound as a black oil.

### C) C-(1-m-Tolyl-cyclopent-3-enyl)-methylamine hydrochloride

A solution of 1-m-Tolyl-cyclopent-3-enecarbonitrile (2.25 g, 11.0 mmol) in tetrahydrofurane (10 ml) was added to a stirred solution of Lithium aluminium hydride (1.3 g, 33.1 mmol) in tetrahydrofurane (10 ml) at 0° C. over a period of 30 minutes. After the addition was complete, the mixture was stirred for 1 h at 0° C., then heated to 40° C. and stirred for 16 h at 40° C. After cooling, the reaction mixture was quenched carefully with a mixture of water and 10% aqueous Sodium hydroxide solution and extracted into ethyl acetate. The organic phase was filtered, then the filtrate was dried and concentrated in vacuo. The residue was dissolved in diethylether (2 ml) and treated with 2M Hydrogen chloride in diethylether (6.1 ml, 12 mmol) at 0° C., The precipitate was filtered and ddried to give the title compound as a white solid.

MS (ES⁺): 188 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.66 min.

### D) (1-m-Tolyl-cyclopent-3-enylmethyl)-carbamic acid tert-butyl ester

To a solution of C-(1-m-Tolyl-cyclopent-3-enyl)-methylamine hydrochloride (1.0 g, 4.47 mmol) and Triethylamine (1.87 ml, 13.4 mmol) in dichloromethane (10 ml) was added a solution of Di-tert-Butyl dicarbonate (2.93 g, 13.4 mmol) in dichloromethane (5 ml). The resulting mixture was stirred at room temperature for 4 h. The mixture was partitioned between dichloromethane and saturated aqueous Sodium bicarbonate solution. The organic phase was dried and concentrated in vacuo. The residue was purified by flash chromatography. Fractions containing the product were concentrated in vacuo to give the title compound as a yellow oil.

MS (ES⁺): 232 [M-tBu+H]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 5.47 min.

### E) (3-Hydroxy-1-m-tolyl-cyclopentylmethyl)-carbamic acid tert-butyl ester

To a solution of Borane dimethyl sulfide complex solution (2.0 M in tetrahydrofurane, 3.7 ml, 7.4 mmol) in tetrahydrofurane (25 ml) was added a solution of (1-m-Tolyl-cyclopent-3-enylmethyl)-carbamic acid tert-butyl ester (1.81 g, 6.17 mmol) in tetrahydrofurane (5 ml) at 0° C. under nitrogen atmosphere. After the addition was complete, the mixture was stirred at room temperature for 16 h. The reaction mixture was cooled to 0° C., then 3N Sodium hydroxide solution (2.5 ml, 7.4 mmol) was added dropwise, followed by addition of 30% solution of hydrogen peroxide in water (3.5 ml, 34.0 mmol). The resulting mixture was stirred at 40° C. for 1 h. After cooling, the mixture was treated with 10% aqueous sodium thiosulfate solution. The separated organic layer was diluted with dichloromethane and washed with 1N Hydrochloric acid. The organic layer was dried and concentrated in vacuo. The residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were partitioned between dichloromethane and saturated aqueous sodium bicarbonate solution. The organic layer was dried and concentrated in vacuo to give the title compound as a colourless oil as a mixture of diastereomers.

MS (ES⁺): 329 [M+Na]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 3.34 min+3.47 min.

### F) (3-Oxo-1-m-tolyl-cyclopentylmethyl)-carbamic acid tert-butyl ester

A solution of (3-Hydroxy-1-m-tolyl-cyclopentylmethyl)-carbamic acid tert-butyl ester (120 mg, 0.393 mmol) in dichloromethane (1 ml) was added to a suspension of Pyridinium chlorochromate (144 mg, 0.668 mmol) and molecular sieves in dichloromethane (1 ml). The resulting mixture was stirred at 40° C. for 2 h. The mixture was partitioned between dichloromethane and saturated aqueous Sodium bicarbonate solution. The organic phase was dried and concentrated in vacuo. The residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were partitioned between dichloromethane and saturated aqueous sodium bicarbonate solution. The organic layer was dried and concentrated in vacuo to give the title compound as a colourless oil.

MS (ES⁺): 326 [M+Na]⁺

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 3.57 min.

### G) [1-m-Tolyl-3-(3-trifluoromethyl-5,6-dihydro-8H-[1,2,4]triazolof-4,3-alovrazin-7-yl)-cyclopentylmethyl]-carbamic acid tert-butyl ester

To a solution of (3-Oxo-1-m-tolyl-cyclopentylmethyl)-carbamic acid tert-butyl ester (55 mg, 0.181 mmol) in dichloromethane (2 ml) was added 3-Trifluoromethyl-5,6,7,8-tetrahydr o-[1,2,4]triazolo[4,3-a]pyrazine (52 mg, 0.272 mmol) and acetic acid (10 μl, 0.181 mmol). The resulting mixture was stirred at room temperature for 1 h, then Sodium triacetoxyborohydride (61 mg, 0.272 mmol) was added. The resulting mixture was stirred at room temperature for 16 h. The mixture concentrated in vacuo. The residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were partitioned between dichloromethane and saturated aqueous sodium bicarbonate solution. The organic layer was dried and concentrated in vacuo to give the title compound as a white solid.

MS (ES⁺): 480 [M+H]⁺

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 3.11 min.

### H) C-[1-m-Tolyl-3-(3-trifluoromethyl-5,6-dihydro-8H-[1,2,4]triazolo[4,3-a]pyrazin-7-yl)-cyclopentyl]-methylamine hydrochloride

Trifluoroacetic acid (468 μl) was added to a solution of [1-m-Tolyl-3-(3-trifluoromethyl-5,6-dihydro-8H-[1,2,4]triazolo[4,3-a]pyrazin-7-yl)-cyclopentylmethyl]carbamic acid tert-butyl ester (63 mg, 0.122 mmol) in dichloromethane (1 mL). The reaction mixture was stirred at room temperature for 2 h. The mixture was partitioned between dichloromethane and saturated aqueous sodium bicarbonate solution. The organic layer was dried and concentrated in vacuo. The residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were partitioned between dichloromethane and saturated aqueous sodium bicarbonate solution. The organic layer was dried and concentrated in vacuo. The residue was dissolved in methanol and treated with an excess of 2M hydrogen chloride in methanol. Removal of the volatiles gave the title compound as a white solid.

MS (ES⁺): 380 [M+H]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 2.71 min.

### Example W1

### 2-[cis-4-Aminomethyl-4-(3-chloro-phenyl)-cyclohexyl]-6-phenyl-2H-pyridazin-3-one hydrochloride

The title compound was prepared according to Scheme W.

### A) [1-(cis-3-Chloro-phenyl)-4-(6-oxo-3-phenyl-6H-pyridazin-1-yl)-cyclohexylmethyl]-carbamic acid tert-butyl ester

To a solution of a mixture of [1-(cis-3-Chloro-phenyl)-4-hydroxy-cyclohexylmethyl]-carbamic acid tert-butyl ester and [1-(trans-3-Chloro-phenyl)-4-hydroxy-cyclohexylmethyl]-carbamic acid tert-butyl ester (200 mg, 1.16 mmol), 6-Phenyl-3-pyridazinone (481 mg, 1.39 mmol) and Triphenylphosphine polymer bound (3 mmol/g, 620 mg, 1.86 mmol) in tetrahydrofurane (5 ml), was added dropwise Diethyl azodicarboxylate (298 μl, 1.86 mmol) at 0° C. under argon atmosphere. The reaction mixture was stirred for 4 h at 0° C. The mixture was filtered, then the filtrate was partitioned between ethyl acetate and 2N aqueous Hydrochloric acid. The organic layer was dried and concentrated in vacuo to give a mixture of [1-(cis-3-Chloro-phenyl)-4-(6-oxo-3-phenyl-6H-pyridazin-1-yl)-cyclohexylmethyl]-carbamic acid tert-butyl ester and [1-(trans-3-Chloro-phenyl)-4-(6-oxo-3-phenyl-6H-pyridazin-1-yl)-cyclohexylmethyl]-carbamic acid tert-butyl ester. The residue was purified by prep. HPLC (Waters SunFire Prep C18 ODB 5 μm 19×50 mm, flow 20 ml/min, 15 min method (0-2.5 min 20% ACN, 2.5-12.5 min 20-100% ACN, 12.5-15.0 min 100% ACN). Fractions containing the product were concentrated in vacuo, then partitioned between dichloromethane and saturated aqueous sodium bicarbonate solution. The organic layer was dried and concentrated in vacuo to give the title compound as a white solid.

MS (ES⁺): 517 [M+Na]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 4.28 min.

### B) 2-[cis-4-Aminomethyl-4-(3-chloro-phenyl)-cyclohexyl]-6-phenyl-2H-pyridazin-3-one hydrochloride

Trifluoroacetic acid (0.9 ml) was added to a solution of [1-(cis-3-Chloro-phenyl)-4-(6-oxo-3-phenyl-6H-pyridazin-1-yl)-cyclohexylmethyl]-carbamic acid tert-butyl ester (90 mg, 0.179 mmol) in dichloromethane (2 mL). The reaction mixture was stirred at room temperature for 1 h. The mixture was partitioned between dichloromethane and saturated aqueous sodium bicarbonate solution. The organic layer was dried and concentrated in vacuo. The residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were partitioned between dichloromethane and saturated aqueous sodium bicarbonate solution. The organic layer was dried and concentrated in vacuo. The residue was dissolved in methanol and treated with an excess of 2M hydrogen chloride in methanol. Removal of the volatiles gave the title compound as a white solid.

MS (ES⁺): 394 [M+H]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 2.72 min.

### Example W2

### 2-[trans-4-Aminomethyl-4-(3-chloro-phenyl)-cyclohexyl]-6-phenyl-2H-pyridazin-3-one hydrochloride

The title compound was prepared analogously as described in Example W1, step B from [1-(trans-3-Chloro-phenyl)-4-(6-oxo-3-phenyl-6H-pyridazin-1-yl)-cyclohexylmethyl]-carbamic acid tert-butyl ester.

MS (ES⁺): 394 [M+H]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 2.78 min.

### Example W3

### 1-[4-(5-Bromo-pyridin-2-yloxy)-1-(3-chloro-phenyl)-cyclohexyl]-methylamine

The title compound was prepared analogously as described in Example W1, using 5-Bromopyridin-2-one instead of 6-Phenyl-3-pyridazinone.

MS (ES⁺): 395 [M+H]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 3.19 min.

### Example W4

### 2-[trans-4-Aminomethyl-4-(3-chloro-phenyl)-cyclohexyl]-2H-pyridazin-3-one hydrochloride

The title compound was prepared analogously as described in Example W2, using 3(2H)-Pyridazinone instead of 6-Phenyl-3-pyridazinone.

MS (ES⁺): 318 [M+H]⁺.

HPLC (Nucleosil, 6 min method (0-3 min 5-95% ACN, 3.5-5.5 min 95% ACN, 5.5-5.55 min 95-5% ACN, 5.55-6 min 5% ACN): 4.01 min.

### Example W5

### 2-[cis-4-Aminomethyl-4-(3-chloro-phenyl)-cyclohexyl]-2H-pyridazin-3-one hydrochloride

The title compound was prepared analogously as described in Example W1, using 3(2H)-Pyridazinone instead of 6-Phenyl-3-pyridazinone.

MS (ES⁺): 318 [M+H]⁺.

HPLC (Nucleosil, 6 min method (0-3 min 5-95% ACN, 3.5-5.5 min 95% ACN, 5.5-5.55 min 95-5% ACN, 5.55-6 min 5% ACN): 3.97 min.

### Example W6

### 2-[cis-4-Aminomethyl-4-(3-chloro-phenyl)-cyclohexyl]-6-methyl-2H-pyridazin-3-one hydrochloride

The title compound was prepared analogously as described in Example W1, using 6-Methyl-3-pyripazinone instead of 6-Phenyl-3-pyridazinone.

MS (ES⁺): 332 [M+H]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 2.87 min.

### Example W7

### 2-[cis-4-Aminomethyl-4-(3-chloro-phenyl)-cyclohexyl]-3-oxo-6-phenyl-2,3-dihydro-pyridazine-4-carboxylic acid amide

The title compound was prepared analogously as described in Example W1, using 3-Oxo-6-phenyl-2,3-dihydro-pyridazine-4-carboxylic acid amide instead of 6-Phenyl-3-pyridazinone.

MS (ES⁺): 438 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.33 min.

### Example W8

### 2-[4-Aminomethyl-4-(3-chloro-phenyl)-cyclohexyl]-3-oxo-6-phenyl-2,3-dihydro-pyridazine-4-carboxylic acid ethyl ester

The title compound was prepared analogously as described in Example W1, using 3-Oxo-6-phenyl-2,3-dihydro-pyridazine-4-carboxylic acid ethyl ester instead of 6-Phenyl-3-pyridazinone.

MS (ES⁺): 466 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.59 min.

### Example W9

### 3-[cis-4-Aminomethyl-4-(3-chloro-phenyl)-cyclohexyloxy]-6-phenyl-pyridazine-4-carboxylic acid ethyl ester

The title compound was prepared analogously as described in Example W1, using 3-Oxo-6-phenyl-2,3-dihydro-pyridazine-4-carboxylic acid ethyl ester instead of 6-Phenyl-3-pyridazinone.

MS (ES⁺): 466 [M+H]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 3.40 min.

### Example W10

### 2-[cis-4-Aminomethyl-4-(3-chloro-phenyl)-cyclohexyl]-3-oxo-6-phenyl-2,3-dihydro-pyridazine-4-carboxylic acid

The title compound was prepared analogously as described in Example W1, step A using 3-Oxo-6-phenyl-2,3-dihydro-pyridazine-4-carboxylic acid ethyl ester instead of 6-Phenyl-3-pyridazinone to afford 2-[4-(tert-Butoxycarbonylamino-methyl)-4-(cis-3-chloro-phenyl)-cyclohexyl]-3-oxo-6-phenyl-2,3-dihydro-pyridazine-4-carboxylic acid ethyl ester followed by step

### B) 2-[4-(tert-Butoxycarbonylamino-methyl)-4-(cis-3-chloro-phenyl)-cyclohexyl]-3-oxo-6-phenyl-2,3-dihydro-pyridazine-4-carboxylic acid

To a solution of 2-[4-(tert-Butoxycarbonylamino-methyl)-4-(cis-3-chloro-phenyl)-cyclohexyl]-3-oxo-6-phenyl-2,3-dihydro-pyridazine-4-carboxylic acid ethyl ester (100 mg, 0.177 mmol) in tetrahydrofurane (1 ml) and water (1 ml) was added Lithium hydroxide hydrate (37 mg, 0.883 mmol). The mixture was stirred at 60° C. for 3 h. The mixture was partitioned between dichloromethane and 1N Hydrochloric acid. The organic layer was dried and concentrated in vacuo to give the title compound as an orange solid.

MS (ES⁺): 560 [MA-Na]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 4.71 min.

### C) 2-[cis-4-Aminomethyl-4-(3-chloro-phenyl)-cyclohexyl]-3-oxo-6-phenyl-2,3-dihydro-pyridazine-4-carboxylic acid

Trifluoroacetic acid (21 μl) was added to a solution of 2-[4-(tert-Butoxycarbonylamino-methyl)-4-(cis-3-chloro-phenyl)-cyclohexyl]-3-oxo-6-phenyl-2,3-dihydro-pyridazine-4-carboxylic acid (15 mg, 0.028 mmol) in dichloromethane (1 mL). The reaction mixture was stirred at room temperature for 1 h. The mixture was concentrated in vacuo. The residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were partitioned between dichloromethane and saturated aqueous sodium bicarbonate solution. The organic layer was dried and concentrated in vacuo to give the title compound as a yellow solid.

MS (ES⁺): 438 [M+H]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 3.03 min.

### Example WA1

### 2-[cis-4-Aminomethyl-4-(3-chloro-phenyl)-cyclohexyl]-6-(3-methanesulfonyl-phenyl)-2H-pyridazin-3-one

The title compound was prepared according to Scheme W.

### A) [4-(3-Bromo-6-oxo-6H-pyridazin-1-yl)-1-(cis-3-chloro-phenyl)-cyclohexylmethyl]-carbamic acid tert-butyl ester

To a solution of [1-(cis-3-Chloro-phenyl)-4-hydroxy-cyclohexylmethyl]-carbamic acid tert-butyl ester (1.11 g, 3.21 mmol), 6-Bromo-2H-pyridazin-3-one (510 mg, 2.91 mmol) and Triphenylphosphine (917 mg, 3.50 mmol) in tetrahydrofurane (40 ml), was added dropwise Diethyl azodicarboxylate (750 μl, 4.66 mmol) at room temperature under nitrogen atmosphere. The reaction mixture was stirred for 16 h at room temperature. The mixture was partitioned between dichloromethane and saturated aqueous sodium bicarbonate solution. The organic layer was dried and concentrated in vacuo to give a mixture of [4-(3-Bromo-6-oxo-6H-pyridazin-1-yl)-1-(cis-3-chloro-phenyl)-cyclohexylmethyl]-carbamic acid tert-butyl ester and [4-(6-Bromo-pyridazin-3-yloxy)-1-(cis-3-chloro-phenyl)-cyclohexylmethyl]-carbamic acid tert-butyl ester which was separated by prep. HPLC (Waters SunFire Prep C18 ODB 5 μm 30×100 mm, flow 40 ml/min, 45 min method (0-2.5 min 20% ACN, 2.5-42.5 min 20-100% ACN, 42.5-45.0 min 100% ACN). Fractions containing the product were partitioned between dichloromethane and saturated aqueous sodium bicarbonate solution. The organic layer was dried and concentrated in vacuo to give the title compound as a white solid.

MS (ES⁺): 519 [M+Na]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 4.06 min.

### B) {1-(cis-3-Chloro-phenyl)-4-[3-(3-methanesulfonyl-phenyl)-6-oxo-6H-pyridazin-1-yl]-cyclohexylmethyl}-carbamic acid tert-butyl ester

To a suspension of [4-(3-Bromo-6-oxo-6H-pyridazin-1-yl)-1-(cis-3-chloro-phenyl)-cyclohexylmethyl]-carbamic acid tert-butyl ester (50 mg, 0.101 mmol) in 1,2-Dimethoxyethane (1 ml) were added 3-Methylsulfonylphenylboronic acid (23 mg, 0.111 mmol), Tetrakis(triphenylphosphine)palladium(0) (6 mg, 0.005 mmol) and 10% aqueous sodium carbonate solution (0.5 ml, 0.19 mmol) under nitrogen atmosphere. The reaction mixture was stirred for 16 h at 80° C. The mixture was partitioned between dichloromethane and saturated aqueous sodium bicarbonate solution. The organic layer was dried and concentrated in vacuo. The residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were partitioned between dichloromethane and saturated aqueous sodium bicarbonate solution. The organic layer was dried and concentrated in vacuo to give the title compound as a white solid.

MS (ES⁺): 595 [M+Na]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 3.91 min.

### C) 2-[cis-4-Aminomethyl-4-(3-chloro-phenyl)-cyclohexyl]-6-(3-methanesulfonyl-phenyl)-2H-pyridazin-3-one

Trifluoroacetic acid (0.5 ml) was added to a solution of {1-(cis-3-Chloro-phenyl)-4-[3-(3-methanesulfonyl-phenyl)-6-oxo-6H-pyridazin-1-yl]-cyclohexylmethyl}-carbamic acid tert-butyl ester (10 mg, 0.017 mmol) in dichloromethane (2 mL). The reaction mixture was stirred at room temperature for 1 h. The mixture concentrated in vacuo. The residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were partitioned between dichloromethane and saturated aqueous sodium bicarbonate solution. The organic layer was dried and concentrated in vacuo to give the title compound as a colourless solid.

MS (ES⁺): 472 [M+H]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 2.58 min.

### Example WA2

### 2-[cis-4-Aminomethyl-4-(3-chloro-phenyl)-cyclohexyl]-6-pyridin-3-yl-2H-pyridazin-3-one hydrochloride

The title compound was prepared analogously as described in Example WA1, step A followed by step

### B) [1-(cis-3-Chloro-phenyl)-4-(6-oxo-3-pyridin-3-v1-6H-pyridazin-1-yl)-cyclohexylmethyl]-carbamic acid tert-butyl ester

A mixture of [4-(3-Bromo-6-oxo-6H-pyridazin-1-yl)-1-(cis-3-chloro-phenyl)-cyclohexylmethyl]-carbamic acid tert-butyl ester (36 mg, 0.072 mmol), 3-Pyridineboronic acid (27 mg, 0.217 mmol), Bis(triphenylphosphine)palladium(II) chloride (5 mg, 0.007 mmol) and Cesium carbonate (47 mg, 0.145 mmol) in Dimethylacetamide/water/ethanol 7:3:2 (1 ml) was treated with microwave at 150° C. for 150 seconds. The mixture was partitioned between dichloromethane and saturated aqueous sodium bicarbonate solution. The organic layer was dried and concentrated in vacuo. The residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were partitioned between dichloromethane and saturated aqueous sodium bicarbonate solution. The organic layer was dried and concentrated in vacuo to give the title compound as a white solid.

MS (ES⁺): 495 [M+H]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 3.16 min.

### C) 2-[cis-4-Aminomethyl-4-(3-chloro-phenyl)-cyclohexyl]-6-pyridin-3-v1-2H-pyridazin-3-one hydrochloride

Trifluoroacetic acid (21 μl) was added to a solution of [1-(cis-3-Chloro-phenyl)-4-(6-oxo-3-pyridin-3-yl-6H-pyridazin-1-yl)-cyclohexylmethyl]-carbamic acid tert-butyl ester (15 mg, 0.027 mmol) in dichloromethane (1 mL). The reaction mixture was stirred at room temperature for 1 h. The mixture was concentrated in vacuo. The residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were partitioned between dichloromethane and saturated aqueous sodium bicarbonate solution. The organic layer was dried and concentrated in vacuo. The residue was treated with an excess of 2M hydrogen chloride in methanol. Removal of the volatiles gave the title compound as a white solid.

MS (ES⁺): 395 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.70 min.

### Example WA3

### 2-[cis-4-Aminomethyl-4-(3-chloro-phenyl)-cyclohexyl]-6-o-tolyl-2H-pyridazin-3-one

The title compound was prepared analogously as described in Example WA2, using o-Tolylboronic acid instead of 3-Pyridineboronic acid.

MS (ES⁺): 408 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.25 min.

### Example WA4

### 2-[cis-4-Aminomethyl-4-(3-chloro-phenyl)-cyclohexyl]-6-pyridin-4-yl-2H-pyridazin-3-one

The title compound was prepared analogously as described in Example WA2, using 4-Pyridineboronic acid instead of 3-Pyridineboronic acid.

MS (ES⁺): 395 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.71 min.

### Example WA5

### 2-[cis-4-Aminomethyl-4-(3-chloro-phenyl)-cyclohexyl]-6-pyrim idin-5-yl-2H-pyridazin-3-one

The title compound was prepared analogously as described in Example WA2, using Pyrimidine-5-boronic acid instead of 3-Pyridineboronic acid.

MS (ES⁺): 396 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.12 min.

### Example WA6

### 2-[cis-4-Aminomethyl-4-(3-chloro-phenyl)-cyclohexyl]-6-(2-dimethylamino-pyrimidin-5-yl)-2H-pyridazin-3-one

The title compound was prepared analogously as described in Example WA2, using 1,2-Dimethyaminopyrimidine-5-boronic acid pinacol ester instead of 3-Pyridineboronic acid.

MS (ES⁺): 438 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.98 min.

### Example WA7

### 2-[cis-4-Aminomethyl-4-(3-chloro-phenyl)-cyclohexyl]-6-m-tolyl-2H-pyridazin-3-one

The title compound was prepared analogously as described in Example WA2, using m-Tolyboronic acid instead of 3-Pyridineboronic acid.

MS (ES⁺): 408 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.85 min.

### Example WA8

### 2-[cis-4-Aminomethyl-4-(3-chloro-phenyl)-cyclohexyl]-6-p-tolyl-2H-pyridazin-3-one

The title compound was prepared analogously as described in Example WA2, using p-Tolyboronic acid instead of 3-Pyridineboronic acid.

MS (ES⁺): 408 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.90 min.

### Example WA9

### 2-[cis-4-Aminomethyl-4-(3-chloro-phenyl)-cyclohexyl]-6-cyclopropyl-2H-pyridazin-3-one

The title compound was prepared analogously as described in Example WA2, using Cyclopropylboronic acid instead of 3-Pyridineboronic acid.

MS (ES⁺): 358 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.03 min.

### Example WA10

### 4-{1-[cis-4-Aminomethyl-4-(3-chloro-phenyl)-cyclohexyl]-6-oxo-1,6-dihydro-pyridazin-3-yl}-benzoic acid hydrochloride

The title compound was prepared analogously as described in Example WA2, step A to B using 4-Ethoxycarbonylphenylboronic acid instead of 3-Pyridineboronic acid followed by step

### C) 4-{1-[4-(tert-Butoxycarbonylamino-methyl)-4-(cis-3-chloro-phenyl)-cyclohexyl]-6-oxo-1,6-dihydro-pyridazin-3-yl}-benzoic acid

To a solution of 4-{1-[4-(tert-Butoxycarbonylamino-methyl)-4-(cis-3-chloro-phenyl)-cyclohexyl]-6-oxo-1,6-dihydro-pyridazin-3-yl}-benzoic acid ethyl ester (30 mg, 0.053 mmol) in tetrahydrofurane (0.5 ml) and water (0.5 ml) was added Lithium hydroxide hydrate (5.6 mg, 0.132 mmol). The mixture was stirred at 60° C. for 3 h. The mixture was partitioned between dichloromethane and 1N Hydrochloric acid. The organic layer was dried and concentrated in vacuo to give the title compound as a white solid.

MS (ES⁺): 561 [M+Na]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 3.82 min.

### D) 4-{1-[cis-4-Aminomethyl-4-(3-chloro-phenyl)-cyclohexyl]-6-oxo-1,6-dihydro-pyridazin-3-yl}-benzoic acid hydrochloride

Trifluoroacetic acid (29 μl) was added to a solution of 4-{1-[4-(tert-eutoxycarbonylamino-methyl)-4-(cis-3-chloro-phenyl)-cyclohexyl]-6-oxo-1,6-dihydro-pyridazin-3-yl}-benzoic acid (20 mg, 0.037 mmol) in dichloromethane (1 mL). The reaction mixture was stirred at room temperature for 1 h. The mixture was concentrated in vacuo. The residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were concentrated in vacuo. The residue was treated with 4M hydrogen chloride in dioxane. Lyophilization of the volatiles gave the title compound as a white solid.

MS (ES⁺): 438 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.54 min.

### Example WA11

### 3-{1-[cis-4-Aminomethyl-4-(3-chloro-phenyl)-cyclohexyl]-6-oxo-1,6-dihydro-pyridazin-3-yl}-benzoic acid hydrochloride

The title compound was prepared analogously as described in Example WA10, using 3-Methoxycarbonylphenylboronic acid instead of 4-Ethoxycarbonylphenylboronic acid.

MS (ES⁺): 438 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.60 min.

### Example WA12

### 5-{1-[cis-4-Aminomethyl-4-(3-chloro-phenyl)-cyclohexyl]-6-oxo-1,6-dihydro-pyridazin-3-yl}-pyridine-2-carboxylic acid hydrochloride

The title compound was prepared analogously as described in Example WA10, using 2-Methylcarboxy-pyridine-5-boronic acid pinacol ester instead of 4-Ethoxycarbonyl-phenylboronic acid.

MS (ES⁺): 439 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.87 min.

### Example WA13

### 2-[cis-4-Aminomethyl-4-(3-chloro-phenyl)-cyclohexyl]-6-(4-methanesulfonyl-phenyl)-2H-pyridazin-3-one

The title compound was prepared analogously as described in Example WA2, using 4-Methanesulfonylphenyl boronic acid instead of 3-Pyridineboronic acid.

MS (ES⁺): 472 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.54 min.

### Example WA14

### 2-[cis-4-Aminomethyl-4-(3-chloro-phenyl)-cyclohexyl]-6-(6-morpholin-4-yl-pyridin-3-yl)-2H-pyridazin-3-one

The title compound was prepared analogously as described in Example WA2, using 6-(Morpholin-4-yl)pyridine-3-boronic acid pinacol ester instead of 3-Pyridineboronic acid.

MS (ES⁺): 481 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.01 min.

### Example WA15

### 2-[cis-4-Aminomethyl-4-(3-chloro-phenyl)-cyclohexyl]-6-quinolin-3-yl-2H-pyridazin-3-one

The title compound was prepared analogously as described in Example WA2, using 3-Quinolineboronic acid pinacol ester instead of 3-Pyridineboronic acid.

MS (ES⁺): 439 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.87 min.

### Example WA16

### 2-[cis-4-Aminomethyl-4-(3-chloro-pherwl)-cyclohexyl]-6-isoquinolin-4-yl-2H-pyridazin-3-one

The title compound was prepared analogously as described in Example WA2, using 4-Isoquinolineboronic acid pinacol ester instead of 3-Pyridineboronic acid.

MS (ES⁺): 439 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.87 min.

### Example WA17

### 2-[cis-4-Aminomethyl-4-(3-chloro-phenyl)-cyclohexyl]-6-(2-amino-pyrimidin-5-yl)-2H-Pyridazin-3-one

The title compound was prepared analogously as described in Example WA2, using 2-Aminopyrimidine-5-boronic acid instead of 3-Pyridineboronic acid.

MS (ES⁺): 411 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.78 min.

### Example WA18

### 2-[cis-4-Aminomethyl-4-(3-chloro-phenyl)-cyclohexyl]-6-(6-methoxy-pyridin-3-yl)-2H-pyridazin-3-one

The title compound was prepared analogously as described in Example WA2, using 2-Methoxy-5-pyridineboronic acid instead of 3-Pyridineboronic acid.

MS (ES⁺): 426 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.04 min.

### Example WA19

### 2-[cis-4-Aminomethyl-4-(3-chloro-phenyl)-cyclohexyl]-6-(6-amino-pyridin-3-yl)-2H-pyridazin-3-one

The title compound was prepared analogously as described in Example WA2, using 2-Aminopyridine-5-boronic acid pinacol ester instead of 3-Pyridineboronic acid.

MS (ES⁺): 410 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.62 min.

### Example WA20

### 3-{1-[cis-4-Aminomethyl-4-(3-chloro-phenyl)-cyclohexyl]-6-oxo-1,6-dihydro-pyridazin-3-yl}-N,N-dimethyl-benzamide

The title compound was prepared analogously as described in Example WA2, using 3-Dimethylcarbamoylphenylboronic acid instead of 3-Pyridineboronic acid.

MS (ES⁺): 465 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.47 min.

### Example WA21

### 2-[cis-4-Aminomethyl-4-(3-chloro-phenyl)-cyclohexyl]-6-(5-methyl-pyridin-3-yl)-2H-pyridazin-3-one

The title compound was prepared analogously as described in Example WA2, using 5-Methylpyridine-3-boronic acid instead of 3-Pyridineboronic acid.

MS (ES⁺): 410 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 1.85 min.

### Example WA22

### 2-[cis-4-Aminomethyl-4-(3-chloro-phenyl)-cyclohexyl]-6-(5-methanesulfonyl-pyridin-3-yl)-2H-pyridazin-3-one

The title compound was prepared analogously as described in Example WA2, using 5-Methanesulfonylpyridine-3-boronic acid instead of 3-Pyridineboronic acid.

MS (ES⁺): 473 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.36 min.

### Example WA23

### 3-{1-[cis-4-Aminomethyl-4-(3-chloro-phenyl)-cyclohexyl]-6-oxo-1,6-dihydro-pyridazin-3-yl}-benzamide

The title compound was prepared analogously as described in Example WA2, using 3-Carbamoylphenylboronic acid instead of 3-Pyridineboronic acid.

MS (ES⁺): 465 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.39 min.

### Example WA24

### 2-[cis-4-Aminomethyl-4-(3-chloro-phenyl)-cyclohexyl]-6-(5-methoxy-pyridin-3-yl)-2H-pyridazin-3-one

The title compound was prepared analogously as described in Example WA2, using 3-Methoxypyridine-5-boronic acid pinacol ester instead of 3-Pyridineboronic acid.

MS (ES⁺): 426 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.84 min.

### Example WA25

### 2-[cis4-Aminomethyl-4-(3-chloro-phenyl)-cyclohexyl]-6-(3-amino-phenyl)-2H-pyridazin-3-one tetrahydrochloride

The title compound was prepared analogously as described in Example WA2, using 3-Aminophenylboronic acid monohydrate instead of 3-Pyridineboronic acid.

MS (ES⁺): 409 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.91 min.

### Example WA26

### 5-{1-[cis-4-Aminomethyl-4-(3-chloro-phenyl)-cyclohexyl]-6-oxo-1,6-dihydro-pyridazin-3-yl}-nicotinic acid dihydrochloride

The title compound was prepared analogously as described in Example WA2, using 5-(Methoxycarbonyl)pyridine-3-boronic acid instead of 3-Pyridineboronic acid.

MS (ES⁺): 439 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.97 min.

### Example WA27

### 4-{1-[cis-4-Aminomethyl-4-(3-chloro-phenyl)-cyclohexyl]-6-oxo-1,6-dihydro-pyridazin-3-yl}-benzenesulfonamide dihydrochloride

The title compound was prepared analogously as described in Example WA2, using 4-Aminosulfonylpyridine-3-boronic acid instead of 3-Pyridineboronic acid.

MS (ES⁺): 473 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.40 min.

### Example WA28

### 2-[cis-4-Aminomethyl-4-(3-chloro-phenyl)-cyclohexyl]-6-(1H-pyrazol-4-yl)-2H-pyridazin-3-one trihydrochloride

The title compound was prepared analogously as described in Example WA2, using 1-Pyrazole-5-boronic acid instead of 3-Pyridineboronic acid.

MS (ES⁺): 384 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.28 min.

### Example WA29

### 2-[cis-4-Aminomethyl-4-(3-chloro-phenyl)-cyclohexyl]-6-(1-benzyl-1H-pyrazol-4-yl)-2H-pyridazin-3-one dihydrochloride

The title compound was prepared analogously as described in Example WA2, using 1-Benzyl-1H-pyrazole-4-boronic acid instead of 3-Pyridineboronic acid.

MS (ES⁺): 474 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.74 min.

### Example WA30

### 2-[cis-4-Aminomethyl-4-(3-chloro-phenyl)-cyclohexyl]-6-(3-morpholin-4-yl-phenyl)-2H-pyridazin-3-one dihydrochloride

The title compound was prepared analogously as described in Example WA2, using 3-Morpholinophenylboronic acid pinacol ester instead of 3-Pyridineboronic acid.

MS (ES⁺): 480 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.64 min.

### Example WA31

### 2-[cis-4-Aminomethyl-4-(3-chloro-phenyl)-cyclohexyl]-6-(1-methyl-1H-pyrazol-4-yl)-2H-pyridazin-3-one

The title compound was prepared analogously as described in Example WA2, using 1-Methylpyrazole-4-boronic acid pinacol ester instead of 3-Pyridineboronic acid.

MS (ES⁺): 398 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.11 min.

### Example WA32

### 2-[cis-4-Aminomethyl-4-(3-chloro-phenyl)-cyclohexyl]-6-(1H-pyrazol-4-yl)-2H-pyridazin-3-one

The title compound was prepared analogously as described in Example WA2, using 4-Pyrazoleboronic acid pinacol ester instead of 3-Pyridineboronic acid.

MS (ES⁺): 384 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.06 min.

### Example WA33

### 2-[cis-4-Aminomethyl-4-(3-chloro-phenyl)-cyclohexyl]-6-(1-oxy-pyridin-3-O-2H-pyridazin-3-one

The title compound was prepared analogously as described in Example WA2, step A to B followed by step

### C) {1-(cis-3-Chloro-phenyl)-4-[6-oxo-3-(1-oxy-pyridin-4-yl)-6H-pyridazin-1-yl]-cyclohexylmethyl}-carbamic acid tert-butyl ester

To a solution of [1-(cis-3-Chloro-phenyl)-4-(6-oxo-3-pyridin-4-yl-6H-pyridazin-1-yl)-cyclohexylmethyl]-carbamic acid tert-butyl ester (50 mg, 0.101 mmol) in dichloromethane (2 ml) was added m-Chloroperbenzoic acid (25 mg, 0.101 mmol). The mixture was stirred at room temperature for 4 h, then concentrated in vacuo. The residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were partitioned between dichloromethane and saturated aqueous sodium bicarbonate solution. The organic layer was dried and concentrated in vacuo to give the title compound as a white solid.

MS (ES⁺): 511 [MA-H]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 3.53 min.

### D) 2-[4-Aminomethyl-4-(3-chloro-phenyl)-cyclohexyl]-6-(1-oxy-pyridin-4-yl)-2H-pyridazin-3-one

Trifluoroacetic acid (35 μl) was added to a solution of {1-(cis-3-Chloro-phenyl)-4-[6-oxo-3-(1-oxy-pyridin-4-yl)-6H-pyridazin-1-yl]-cyclohexylmethyl}-carbamic acid tert-butyl ester (23 mg, 0.045 mmol) in dichloromethane (2 mL). The reaction mixture was stirred at room temperature for 1 h. The mixture was concentrated in vacuo. The residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were partitioned between dichloromethane and saturated aqueous sodium bicarbonate solution. The organic layer was dried and concentrated in vacuo to give the title compound as a yellow solid.

MS (ES⁺): 411 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.94 min.

### Example WA34

### 3-{1-[cis-4-Aminomethyl-4-(3-chloro-phenyl)-cyclohexyl]-6-oxo-1,6-dihydro-pyridazin-3-yl}-benzenesulfonamide

The title compound was prepared analogously as described in Example WA2, using 3-Aminosulfonylbenzeneboronic acid instead of 3-Pyridineboronic acid.

MS (ES⁺): 473 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.16 min.

### Example WA36

### 2-[cis-4-Aminomethyl-4-(3-chloro-phenyl)-cyclohexyl]-6-(3-hydroxy-phenyl)-2H-pyridazin-3-one

The title compound was prepared analogously as described in Example WA2, using 3-Hydroxybenzeneboronic acid instead of 3-Pyridineboronic acid.

MS (ES⁺): 410 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.26 min.

### Example WA36

### 3-{1-[cis-4-Aminomethyl-4-(3-chloro-phenyl)-cyclohexyl]-6-oxo-1,6-dihydro-pyridazin-3-yl}-benzonitrile

The title compound was prepared analogously as described in Example WA2, using 3-Hydroxybenzeneboronic acid instead of 3-Pyridineboronic acid.

MS (ES⁺): 419 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.36 min.

### Example WA37

### 3-{1-[cis-4-Aminomethyl-4-(3-chloro-phenyl)-cyclohexyl]-6-oxo-1,6-dihydro-pyridazin-3-yl}-N-(2-hydroxy-ethyl)-benzamide dihydrochloride

The title compound was prepared analogously as described in Example WA2, using N-[2-hydroxyethyl]benzamide-3-boronic acid pinacol ester instead of 3-Pyridineboronic acid.

MS (ES⁺): 481 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.99 min.

### Example WA38

### 2-[cis-4-Aminomethyl-4-(3-chloro-phenyl)-cyclohexyl]-6-[3-(morpholine-4-carbonyl)-phenyl]-2H-pyridazin-3-one dihydrochloride

The title compound was prepared analogously as described in Example WA2, using 3-(Morpholine-4-carbonyl)phenylboronic acid instead of 3-Pyridineboronic acid.

MS (ES⁺): 507 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.11 min.

### Example WA39

### 2-[cis-4-Aminomethyl-4-(3-chloro-phenyl)-cyclohexyl]-6-[1-(4-methyl-piperazine-1-carbonyl)-phenyl]-2H-pyridazin-3-one dihydrochloride

The title compound was prepared analogously as described in Example WA2, using 3-(4-methylpiperazine-1-carbonyl)phenyl-boronic acid pinacol ester instead of 3-Pyridineboronic acid.

MS (ES⁺): 520 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.83 min.

### Example WA40

### 3-{1-[cis-4-Aminomethyl-4-(3-chloro-phenyl)-cyclohexyl]-6-oxo-1,6-dihydro-pyridazin-3-yl}-N-methyl-benzamide trihydrochloride

The title compound was prepared analogously as described in Example WA2, using 3-(N-Methylaminocarbonyl)phenyl boronic acid instead of 3-Pyridineboronic acid.

MS (ES⁺): 451 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.05 min.

### Example WA41

### 3-{1-[cis-4-Aminomethyl-4-(3-chloro-phenyl)-cyclohexyl]-6-oxo-1,6-dihydro-pyridazin-3-yl}-N-(3-methoxy-propyl)-benzamide trihydrochloride

The title compound was prepared analogously as described in Example WA2, using 3-(3-methoxypropylcarbamoyl)phenylboronic acid instead of 3-Pyridineboronic acid.

MS (ES⁺): 509 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.16 min.

### Example WA42

### 3-{1-[cis-4-Aminomethyl-4-(3-chloro-phenyl)-cyclohexyl]-6-oxo-1,6-dihydro-pyridazin-3-yl}N-(2-methoxy-ethyl)-benzamide trihydrochloride

The title compound was prepared analogously as described in Example WA2, using 3-(2-methoxyetylaminocarbonyl)benzene boronic acid instead of 3-Pyridineboronic acid.

MS (ES⁺): 495 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.11 min.

### Example WA43

### 3-{1-[cis-4-Aminomethyl-4-(3-chloro-phenyl)-cyclohexyl]-6-oxo-1,6-dihydro-pyridazin-3-yl}-N-(2H-tetrazol-5-yl)-benzamide trihydrochloride

The title compound was prepared analogously as described in Example WA2, using 3-(1H-tetrazol-5-yl-carbomoyl)benzene boronic acid instead of 3-Pyridineboronic acid.

MS (ES⁺): 505 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.99 min.

### Example WB1

### 4-Amino-2-[cis-4-aminomethyl-4-(3-chloro-phenyl)-cyclohexyl]-6-Phenyl-2H-pyridazin-3-one

The title compound was prepared analogously as described in Example W10, step A to B to afford 2-[4-(tert-Butoxycarbonylamino-methyl)-4-(cis-3-chloro-phenyl)-cyclohexyl]-3-oxo-6-phenyl-2,3-dihydro-pyridazine-4-carboxylic acid followed by step

### C) [4-(5-Amino-6-oxo-3-phenyl-6H-pyridazin-1-yl)-1-(cis-3-chloro-phenyl)-cyclohexylmethyl]-carbamic acid tert-butyl ester

To a solution of 2-[4-(tert-Butoxycarbonylamino-methyl)-4-(cis-3-chloro-phenyl)-cyclohexyl]-3-oxo-6-phenyl-2,3-dihydro-pyridazine-4-carboxylic acid (30 mg, 0.056 mmol) in toluene (250 μl) was added Diphenyl phosphoryl azide (9 μl, 0.056 mmol) and Triethylamine (8 μl, 0.056 mmol). The mixture was stirred at 80° C. for 2 h, then water (50 μl) was added and the resulting mixture was stirred at 80° C. for 5 h. The mixture was directly purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were partitioned between dichloromethane and saturated aqueous sodium bicarbonate solution. The organic layer was dried and concentrated in vacuo to give the title compound.

MS (ES⁺): 532 [M+Na]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 4.66 min.

### D) 4-Amino-2-[cis-4-aminomethyl-4-(3-chloro-phenyl)-cyclohexyl]-6-phenyl-2H-pyridazin-3-one

Trifluoroacetic acid (9 μl) was added to a solution of [4-(5-Amino-6-oxo-3-phenyl-6H-pyridazin-1-yl)-1-(cis-3-chloro-phenyl)-cyclohexylmethyl]-carbamic acid tert-butyl ester (6 mg, 0.012 mmol) in dichloromethane (1 mL). The reaction mixture was stirred at room temperature for 1 h. The mixture was concentrated in vacuo. The residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were partitioned between dichloromethane and saturated aqueous sodium bicarbonate solution. The organic layer was dried and concentrated in vacuo to give the title compound.

MS (ES⁺): 409 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.46 min.

### Example WC1

### 2-[cis-4-Aminomethyl-4-(3-chloro-phenyl)-cyclohexyl]-3-oxo-6-phenyl-2,3-dihydro-pyridazine-4-carboxylic acid dimethylamide formate

The title compound was prepared analogously as described in Example W10, step A to B to afford 2-[4-(tert-Butoxycarbonylamino-methyl)-4-(cis-3-chloro-phenyl)-cyclohexyl]-3-oxo-6-phenyl-2,3-dihydro-pyridazine-4-carboxylic acid followed by step

### C) [1-(cis-3-Chloro-phenyl)-4-(5-dimethylcarbamoyl-6-oxo-3-phenyl-6H-pyridazin-1-yl)-cyclohexylmethyl]-carbamic acid tert-butyl ester

To a solution of 2-[4-(tert-Butoxycarbonylamino-methyl)-4-(cis-3-chloro-phenyl)-cyclohexyl]-3-oxo-6-phenyl-2,3-dihydro-pyridazine-4-carboxylic acid (20 mg, 0.037 mmol) in Tetrahydrofurane (150 μl) was added N-Methyl morpholine (12 μl, 0.11 mmol) and Isobutylchloroformate (6 μl, 0.044 mmol) at 0° C. The mixture was stirred at 0° C. for 30 minutes, then Dimethylamine hydrochloride (4 mg, 0.044 mmol) was added and the resulting mixture was stirred at 0° C. for 1 h, then at room temperature for 16 h. The mixture was concentrated in vacuo. The residue was purified by prep. HPLC (Waters SunFire Prep C18 ODB 5 μm 19×50 mm, flow 20 ml/min, 15 min method (0-2.5 min 20% ACN, 2.5-12.5 min 20-100% ACN, 12.5-15.0 min 100% ACN). Fractions containing the product were partitioned between dichloromethane and saturated aqueous sodium bicarbonate solution. The organic layer was dried and concentrated in vacuo to give the title compound as a colourless gum.

MS (ES⁺): 588[M+Na]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 4.32 min.

### D) 2-[cis-4-Aminomethyl-4-(3-chloro-phenyl)-cyclohexyl]-3-oxo-6-phenyl-2,3-dihydro-pyridazine-4-carboxylic acid dimethylamide formate

Trifluoroacetic acid (38 μl) was added to a solution of [1-(cis-3-Chloro-phenyl)-4-(5-dimethylcarbamoyl-6-oxo-3-phenyl-6H-pyridazin-1-yl)-cyclohexylmethyl]-carbamic acid tert-butyl ester (4 mg, 0.007 mmol) in dichloromethane (250 μL). The reaction mixture was stirred at room temperature for 2 h. The mixture was concentrated in vacuo. The residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were concentrated in vacuo to give the title compound as a white solid.

MS (ES⁺): 465 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.94 min.

### Example WC2l

### 2-[cis-4-Aminomethyl-4-(3-chloro-phenyl)-cyclohexyl]-4-(morpholine-4-carbonyl)-6-phenyl-2H-pyridazin-3-one formate

The title compound was prepared analogously as described in Example WC1, using Morpholine instead of Dimethylamine hydrochloride.

MS (ES⁺): 507 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.10 min.

### Example WC3

### 2-[cis-4-Aminomethyl-4-(3-chloro-phenyl)-cyclohexyl]-3-oxo-6-phenyl-2,3-dihydro-pyridazine-4-carboxylic acid methylamide formate

The title compound was prepared analogously as described in Example WC1, using Methylamine (2M solution in Tetrahydrofurane) instead of Dimethylamine hydrochloride.

MS (ES⁺): 452 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.44 min.

### Example WC4

### 2-[cis-4-Aminomethyl-4-(3-chloro-phenyl)-cyclohexyl]-3-oxo-6-phenyl-2,3-dihydro-pyridazine-4-carboxylic acid cyclopropylamide formate

The title compound was prepared analogously as described in Example WC1, using Methylamine (2M solution in Tetrahydrofurane) instead of Dimethylamine hydrochloride.

MS (ES⁺): 478 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.65 min.

### Example WC5

### 2-[cis-4-Aminomethyl-4-(3-chloro-phenyl)-cyclohexyl]-3-oxo-6-phenyl-2,3-dihydro-pyridazine-4-carboxylic acid (2-methoxy-ethyl)-amide

The title compound was prepared analogously as described in Example WC1, using 2-Methoxyethylamine instead of Dimethylamine hydrochloride.

MS (ES⁺): 496 [M+H]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 2.99 min.

### Example WC6

### 2-[cis-4-Aminomethyl-4-(3-chloro-phenyl)-cyclohexyl]-3-oxo-6-phenyl-2,3-dihydro-pitridazine-4-carboxylic acid carbamoylmethyl-amide formate

The title compound was prepared analogously as described in Example WC1, using 2-Amino acetamide instead of Dimethylamine hydrochloride.

MS (ES⁺): 495[M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.14 min.

### Example WC7

### 2-[4-Aminomethyl-4-(3-chloro-phenyl)-cyclohexyl]-4-(3-oxo-piperazine-1-carbonyl)-6-phenyl-2H-pyridazin-3-one formate

The title compound was prepared analogously as described in Example WC1, using 2-piperazin-2-one instead of Dimethylamine hydrochloride.

MS (ES⁺): 520[M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 3.07 min.

### Example WC8

### 2-[4-Aminomethyl-4-(3-chloro-phenyl)-cyclohexyl]-6-phenyl-4-(piperazine-1-carbonyl)-2H-pyridazin-3-one diformate

The title compound was prepared analogously as described in Example WC1, using Bocpiperazine instead of Dimethylamine hydrochloride.

MS (ES⁺): 506[M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.91 min.

### Example WD1

### 2-[cis-4-Aminomethyl-4-(3-chloro-phenyl)-cyclohexyl]-3-oxo-6-phenyl-2,3-dihydro-pyridazine-4-carboxylic acid hydrazide

The title compound was prepared analogously as described in Example W8, step A to afford 2-[4-(tert-Butoxycarbonylamino-methyl)-4-(cis-3-chloro-phenyl)-cyclohexyl]-3-oxo-6-phenyl-2,3-dihydro-pyridazine-4-carboxylic acid ethyl ester followed by step

### B) [1-(cis-3-Chloro-phenyl)-4-(5-hydrazinocarbonyl-6-oxo-3-phenyl-6H-pyridazin-1-yl)-cyclohexylmethyl]-carbamic acid tert-butyl ester

To a solution of 2-[4-(tert-Butoxycarbonylamino-methyl)-4-(cis-3-chloro-phenyl)-cyclohexyl]-3-oxo-6-phenyl-2,3-dihydro-pyridazine-4-carboxylic acid ethyl ester (55 mg, 0.097 mmol) in Ethanol (1 ml) was added Hydrazine hydrate (96 μl, 1.94 mmol). The mixture was refluxed for 2 h. The mixture was partitioned between dichloromethane and saturated aqueous sodium bicarbonate solution. The organic layer was dried and concentrated in vacuo to give the title compound as a yellow solid.

MS (ES⁺): 574[M+Na]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 4.37 min.

### C) 2-[cis-4-Aminomethyl-4-(3-chloro-phenyl)-cyclohexyl]-3-oxo-6-phenyl-2,3-dihydro-pyridazine-4-carboxylic acid hydrazide

Trifluoroacetic acid (56 μl) was added to a solution of [1-(cis-3-Chloro-phenyl)-4-(5-hydrazinocarbonyl-6-oxo-3-phenyl-6H-pyridazin-1-yl)-cyclohexylmethyl]-carbamic acid tert-butyl ester (40 mg, 0.072 mmol) in dichloromethane (2 mL). The reaction mixture was stirred at room temperature for 1 h. The mixture was concentrated in vacuo. The residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were partitioned between dichloromethane and saturated aqueous sodium bicarbonate solution. The organic layer was dried and concentrated in vacuo to give the title compound as a yellow solid.

MS (ES⁺): 452 [M+H]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 3.18 min.

### Example WE1

### 2-[cis-4-Aminomethyl-4-(3-chloro-phenyl)-cyclohexyl]-6-(1H-tetrazol-5-yl)-2H-pyridazin-3-one hydrochloride

The title compound was prepared analogously as described in Example WA1, step A to afford [4-(3-Bromo-6-oxo-6H-pyridazin-1-yl)-1-(cis-3-chloro-phenyl)-cyclohexylmethyl]-carbamic acid tert-butyl ester followed by step

### B) [[1-(cis-3-Chloro-phenyl)-4-(3-cyano-6-oxo-6H-pyridazin-1-yl)-cyclohexylmethyl]-carbamic acid tert-butyl ester

To a solution of [4-(3-Bromo-6-oxo-6H-pyridazin-1-yl)-1-(cis-3-chloro-phenyl)-cyclohexylmethyl]-carbamic acid tert-butyl ester (50 mg, 0.101 mmol) in Dimethylformamide (1 ml) was added Tetrakis(triphenylphosphine)palladium(0) (3.5 mg, 0.003 mmol) and Zinc cyanide (12 mg, 0.101 mmol) under argon atmosphere. The mixture was treated with microwave at 120° C. for 120 seconds. The mixture was filtered. The filtrate was concentrated in vacuo. The residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were lyophilized in vacuo to give the title compound.

MS (ES⁺): 443 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 5.60 min.

### C) {1-(cis-3-Chloro-phenyl)-4-[6-oxo-3-(1H-tetrazol-5-yl)-6H-pyridazin-1-yl]-cyclohexylmethyl}-carbamic acid tert-butyl ester

To a solution of [[1-(cis-3-Chloro-phenyl)-4-(3-cyano-6-oxo-6H-pyridazin-1-yl)-cyclohexylmethyl]-carbamic acid tert-butyl ester (27 mg, 0.061 mmol) in Dimethylformamide (1 ml) was added Sodium azide (48 mg, 0.732 mmol) and Ammonium chloride (39 mg, 0.731 mmol) under argon atmosphere. The mixture was treated with microwave at 120° C. for 15 minutes. The mixture was filtered. The filtrate was concentrated in vacuo to give the title compound.

### D) 2-[cis-4-Aminomethyl-4-(3-chloro-phenyl)-cyclohexyl]-6-(1H-tetrazol-5-yl)-2H-pyridazin-3-one hydrochloride

To {1-(cis-3-Chloro-phenyl)-4-[6-oxo-3-(1H-tetrazol-5-yl)-6H-pyridazin-1-yl]-cyclohexylmethyl}-carbamic acid tert-butyl ester (29 mg, 0.060 mmol) was added 4N hydrogen chloride solution in dioxane (5 ml). The reaction mixture stirred at room temperature for 2 h, then it was concentrated in vacuo. The residue was purified by prep. HPLC (Macherey-Nagel Nucleosil 100-10 C18, flow 40 ml/min, 21 min method (0-2.0 min 5% ACN, 2.0-17.5 min 5-100% ACN, 17.5-19.5 min 100% ACN, 19.5-21.0 min 100-5% ACN). Fractions containing the product were lyophilized in vacuo to give the formate salt of the title compound, which was dissolved in methanol and treated with an excess of 2M hydrogen chloride in methanol. Removal of the volatiles gave the title compound as a white solid.

MS (ES⁺): 386 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 2.45 min.

### Example WF1

### 6-Amino-2-[cis-4-aminomethyl-4-(3-chloro-phenyl)-cyclohexyl]-2H-pyridazin-3-one hydrochloride

The title compound was prepared analogously as described in Example WA1, step A to afford [4-(3-Bromo-6-oxo-6H-pyridazin-1-yl)-1-(cis-3-chloro-phenyl)-cyclohexylmethyl]-carbamic acid tert-butyl ester followed by step

### B) [4-(3-Amino-6-oxo-6H-pyridazin-1-yl)-1-(cis-3-chloro-phenyl)-cyclohexylmethyl]-carbamic acid tert-butyl ester trifluoroacetate

To a solution of [4-(3-Bromo-6-oxo-6H-pyridazin-1-yl)-1-(cis-3-chloro-phenyl)-cyclohexylmethyl]-carbamic acid tert-butyl ester (50 mg, 0.101 mmol) in a mixture of Ethanol (1.4 ml) and water (0.6 ml) was added Sodium Azide (13 mg, 0.202 mmol), Copper iodide (2 mg, 0.010 mmol), Sodium ascorbate (1 mg, 0.005 mmol) and N—N-Dimethylethylenediamine (1.6 μl, 0.015 mmol) under argon atmosphere. The mixture was treated with microwave at 100° C. for 30 minutes. The mixture was filtered. The filtrate was concentrated in vacuo. The residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were lyophilized in vacuo to give the title compound.

MS (ES⁺): 433 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 4.50 min.

### C) 6-Amino-2-[cis-4-aminomethyl-4-(3-chloro-phenyl)-cyclohexyl]-2H-pyridazin-3-one hydrochloride

To [4-(3-Amino-6-oxo-6H-pyridazin-1-yl)-1-(cis-3-chloro-phenyl)-cyclohexylmethyl]-carbamic acid tert-butyl ester trifluoroacetate (33 mg, 0.076 mmol) was added 4N hydrogen chloride solution in dioxane (5 ml). The reaction mixture stirred at room temperature for 2 h, then it was concentrated in vacuo. The residue was purified by prep. HPLC (Macherey-Nagel Nucleosil 100-10 C18, flow 40 ml/min, 21 min method (0-2.0 min 5% ACN, 2.0-17.5 min 5-100% ACN, 17.5-19.5 min 100% ACN, 19.5-21.0 min 100-5% ACN). Fractions containing the product were lyophilized in vacuo to give the formate salt of the title compound, which was dissolved in methanol and treated with an excess of 2M hydrogen chloride in methanol. Removal of the volatiles gave the title compound as a white solid.

MS (ES⁺): 333 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 1.76 min.

### Example WF2

### N-{1-[cis-4-Aminomethyl-4-(3-chloro-phenyl)-cyclohexyl]-6-oxo-1,6-dihydro-pyridazin-3-yl}-acetamide hydrochloride

The title compound was prepared analogously as described in Example WF1, step A to B to afford [4-(3-Amino-6-oxo-6H-pyridazin-1-yl)-1-(cis-3-chloro-phenyl)-cyclohexylmethyl]-carbamic acid tert-butyl ester trifluoroacetate followed by step

### C) [4-(3-Acetylamino-6-oxo-6H-pyridazin-1-yl)-1-(cis-3-chloro-phenyl)-cyclohexylmethyl]-carbamic acid tert-butyl ester

To a solution of [4-(3-Amino-6-oxo-6H-pyridazin-1-yl)-1-(cis-3-chloro-phenyl)-cyclohexylmethyl]-carbamic acid tert-butyl ester trifluoroacetate (18 mg, 0.042 mmol) in Dichloromethane (1.5 ml) was added Triethylamine (29 μl, 0.21 mmol) and Acetylchloride (3.5 μl, 0.05 mmol). The mixture was stirred at room temperature for 2 h. The mixture was filtered. The filtrate was concentrated in vacuo to give the title compound.

### D) N-{1-[cis-4-Aminomethyl-4-(3-chloro-phenyl)-cyclohexyl]-6-oxo-1,6-dihydro-pyridazin-3-yl}-acetamide hydrochloride

To [4-(3-Acetylamino-6-oxo-6H-pyridazin-1-yl)-1-(cis-3-chloro-phenyl)-cyclohexylmethyl]-carbamic acid tert-butyl ester (20 mg, 0.042 mmol) was added 4N hydrogen chloride solution in dioxane (5 ml). The reaction mixture stirred at room temperature for 2 h, then it was concentrated in vacuo. The residue was purified by prep. HPLC (Macherey-Nagel Nucleosil 100-10 C18, flow 40 ml/min, 21 min method (0-2.0 min 5% ACN, 2.0-17.5 min 5-100% ACN, 17.5-19.5 min 100% ACN, 19.5-21.0 min 100-5% ACN). Fractions containing the product were lyophilized in vacuo to give the formate salt of the title compound, which was dissolved in methanol and treated with an excess of 2M hydrogen chloride in methanol. Removal of the volatiles gave the title compound as a white solid.

MS (ES⁺): 375 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 2.12 min.

### Example WF3

### 2-[cis-4-Aminomethyl-4-(3-chloro-phenyl)-cyclohexyl]-6-benzoyl-2H-pyridazin-3-one hydrochloride

The title compound was prepared analogously as described in Example WF1, step A to B to afford [4-(3-Amino-6-oxo-6H-pyridazin-1-yl)-1-(3-chloro-phenyl)-cyclohexylmethyl]-carbamic acid tert-butyl ester trifluoroacetate followed by step

### C)[4-(3-Benzoyl-6-oxo-6H-pyridazin-1-yl)-1-(cis-3-chloro-phenyl)-cyclohexylmethyl]-carbamic acid tert-butyl ester

To a solution of [4-(3-Amino-6-oxo-6H-pyridazin-1-yl)-1-(cis-3-chloro-phenyl)-cyclohexylmethyl]-carbamic acid tert-butyl ester trifluoroacetate (61 mg, 0.141 mmol) in Dichloromethane (3 ml) was added Benzoic acid (26 mg, 0.211 mmol), N-(3-Dimethylaminopropyl)-N′-ethylcarbodiimide (51 μL, 0.282 mmol), 1-Hydroxybenzotriazole hydrate (42 mg, 0.310 mmol) and Triethylamine (98 μl, 0.705 mmol). The mixture was stirred at 40° C. for 48 h. The mixture was filtered. The filtrate was concentrated in vacuo. The residue was purified by prep. HPLC (Macherey-Nagel Nucleosil 100-10 C18, flow 40 ml/min, 21 min method (0-2.0 min 5% ACN, 2.0-17.5 min 5-100% ACN, 17.5-19.5 min 100% ACN, 19.5-21.0 min 100-5% ACN). Fractions containing the product were concentrated in vacuo to give title compound.

MS (ES⁺): 537 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 5.68 min.

### D) 2-[4-Aminomethyl-4-(3-chloro-phenyl)-cyclohexyl]-6-benzoyl-2H-pyridazin-3-one hydrochloride

To [4-(3-Benzoyl-6-oxo-6H-pyridazin-1-yl)-1-(3-chloro-phenyl)-cyclohexylmethyl]-carbamic acid tert-butyl ester (6 mg, 0.011 mmol) was added 4N hydrogen chloride solution in dioxane (5 ml). The reaction mixture stirred at room temperature for 2 h, then it was concentrated in vacuo. The residue was purified by prep. HPLC (Macherey-Nagel Nucleosil 100-10 C18, flow 40 ml/min, 21 min method (0-2.0 min 5% ACN, 2.0-17.5 min 5-100% ACN, 17.5-19.5 min 100% ACN, 19.5-21.0 min 100-5% ACN). Fractions containing the product were lyophilized in vacuo to give the formate salt of the title compound, which was dissolved in methanol and treated with an excess of 2M hydrogen chloride in methanol. Removal of the volatiles gave the title compound as a white solid.

MS (ES⁺): 437 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 3.02 min.

### Example WG1

### 2-[cis-4-Aminomethyl-4-(3-chloro-phenyl)-cyclohexyl]-6-{1-[2-(3,5-dimethyl-1H-pyrazol-4-yl)-ethyl]-1H-[1,2,3]triazol-4-yl}-2H-pyridazin-3-one hydrochloride

The title compound was prepared analogously as described in Example WA1, step A to afford [4-(3-Bromo-6-oxo-6H-pyridazin-1-yl)-1-(cis-3-chloro-phenyl)-cyclohexylmethyl]-carbamic acid tert-butyl ester followed by step

### B) [1-(cis-3-Chloro-phenyl)-4-(3-ethynyl-6-oxo-6H-ovridazin-1-yl)-cyclohexylmethyl]-carbamic acid tert-butyl ester

To a solution of [4-(3-Bromo-6-oxo-6H-pyridazin-1-yl)-1-(cis-3-chloro-phenyl)-cyclohexylmethyl]-carbamic acid tert-butyl ester (50 mg, 0.101 mmol) in Dimethylformamide (1 ml) was added Trimethylsilylacetylene (15 μl, 0.111 mmol), Copper iodide (1 mg, 0.005 mmol), trans-Dichlorobis(triphenylphosphine)palladium(II) (3.5 mg, 0.005 mmol), Triphenylphosphine (5.3 mg, 0.02 mmol) and Diethylamine (157 μl, 1.51 mmol) under argon atmosphere. The mixture was treated with microwave at 120° C. for 30 minutes. The mixture was filtered. The filtrate was concentrated in vacuo. The residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were lyophilized in vacuo to give the title compound.

MS (ES⁺): 442 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 5.61 min.

### C) [1-(cis-3-Chloro-phenyl)-4-(3-{1-[2-(3,5-dimethyl-1H-pyrazol-4-yl)-ethyl]-1H-[1,2,3]triazol-4-yl}-6-oxo-6H-pyridazin-1-yl)-cyclohexylmethyl]-carbamic acid tert-butyl ester

To [1-(cis-3-Chloro-phenyl)-4-(3-ethynyl-6-oxo-6H-pyridazin-1-yl)-cyclohexylmethyl]-carbamic acid tert-butyl ester (25 mg, 0.057 mmol) in a mixture of Dichloromethane (1 ml) and water (1 ml) was added 4-(2-Azidoethyl)-3,5-dimethyl-1H-pyrazole (9.3 mg, 0.057 mmol), Copper sulfate (0.5 mg, 0.003 mmol) and Sodium ascorbate (1.7 mg, 0.008 mmol). The mixture was stirred at room temperature for 16 h. The mixture was filtered. The filtrate was concentrated in vacuo to give the title compound.

### D) 2-[cis-4-Aminomethyl-4-(3-chloro-phenyl)-cyclohexyl]-6-{1-[2-(3,5-dimethyl-1H-pyrazol-4-yl)-ethyl]-1H-[1,2,3]triazol-4-yl}-2H-pyridazin-3-one hydrochloride

To [1-(cis-3-Chloro-phenyl)-4-(3-{1-[2-(3,5-dimethyl-1H-pyrazol-4-yl)-ethyl]-1H-[1,2,3]triazol-4-yl}-6-oxo-6H-pyridazin-1-yl)-cyclohexylmethyl]-carbamic acid tert-butyl ester (34 mg, 0.056 mmol) was added 4N hydrogen chloride solution in dioxane (5 ml). The reaction mixture stirred at room temperature for 2 h, then it was concentrated in vacuo. The residue was purified by prep. HPLC (Macherey-Nagel Nucleosil 100-10 C18, flow 40 ml/min, 21 min method (0-2.0 min 5% ACN, 2.0-17.5 min 5-100% ACN, 17.5-19.5 min 100% ACN, 19.5-21.0 min 100-5% ACN). Fractions containing the product were lyophilized in vacuo to give the formate salt of the title compound, which was dissolved in methanol and treated with an excess of 2M hydrogen chloride in methanol. Removal of the volatiles gave the title compound as a white solid.

MS (ES⁺): 507 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 2.30 min.

### Example WG2

### (4-{1-[cis-4-Aminomethyl-4-(3-chloro-phenyl)-cyclohexyl]-6-oxo-1,6-dihydro-pyridazin-3-yl}-[1,2,3]triazol-1-yl)-acetic acid ethyl ester hydrochloride

The title compound was prepared analogously as described in Example WG1, using Ethylazidoacetate instead of 4-(2-Azidoethyl)-3,5-dimethyl-1H-pyrazole.

MS (ES⁺): 471[M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 2.98 min.

### Example WG3

### (4-{1-[cis-4-Aminomethyl-4-(3-chloro-phenyl)-cyclohexyl]-6-oxo-1,6-dihydro-pyridazin-3-yl}-[1,2,3]triazol-1-yl)-acetic acid hydrochloride

The title compound was prepared analogously as described in Example WG2 followed by step:

### E) (4-{1-[cis-4-Aminomethyl-4-(3-chloro-phenyl)-cyclohexyl]-6-oxo-1,6-dihydro-pyridazin-3-yl}-[1,2,3]triazol-1-yl)-acetic acid hydrochloride

To (4-{1-[cis-4-Aminomethyl-4-(3-chloro-phenyl)-cyclohexyl]-6-oxo-1,6-dihydro-pyridazin-3-yl}-[1,2,3]triazol-1-yl)-acetic acid ethyl ester hydrochloride (6 mg, 0.013 mmol) in dioxane (2 ml) was added 1M aqueous potassium hydroxide solution (1 ml). The mixture was treated with microwave at 120° C. for 5 min. The mixture was evaporated. The residue was purified by prep. HPLC(Macherey-Nagel Nucleosil 100-10 C18, flow 40 ml/min, 21 min method (0-2.0 min 5% ACN, 2.0-17.5 min 5-100% ACN, 17.5-19.5 min 100% ACN, 19.5-21.0 min 100-5% ACN). Fractions containing the product were lyophilized in vacuo to give the formate salt of the title compound, which was dissolved in methanol and treated with an excess of 2M hydrogen chloride in methanol. Removal of the volatiles gave the title compound as a white solid.

MS (ES⁺): 443 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 2.44 min.

### Example WG4

### (4-(1-[cis-4-Aminomethyl-4-(3-chloro-phenyl)-cyclohexyl]-6-oxo-1,6-dihydro-pyridazin-3-yl)-[1,2,3]triazol-1-yl)-acetic acid hydrochloride

The title compound was prepared analogously as described in Example WG2 step A to C followed by step:

### D) (4-{1-[4-(tert-Butoxycarbonylamino-methyl)-4-(cis-3-chloro-phenyl)-cyclohexyl]-6-oxo-1,6-dihydro-pyridazin-3-yl}-[1,2,3]triazol-1-yl)-acetic acid

To (4-{1-[4-(tert-Butoxycarbonylamino-methyl)-4-(cis-3-chloro-phenyl)-cyclohexyl]-6-oxo-1,6-dihydro-pyridazin-3-yl}-[1,2,3]triazol-1-yl)-acetic acid ethyl ester (22 mg, 0.039 mmol) in dioxane (2 ml) was added 1M aqueous potassium hydroxide solution (1.5 ml). The mixture was treated with microwave at 120° C. for 5 min. The mixture was evaporated. The residue was purified by prep. HPLC (Macherey-Nagel Nucleosil 100-10 C18, flow 40 ml/min, 21 min method (0-2.0 min 5% ACN, 2.0-17.5 min 5-100% ACN, 17.5-19.5 min 100% ACN, 19.5-21.0 min 100-5% ACN). Fractions containing the product were lyophilized in vacuo to give the title compound.

MS (ES⁺): 543 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 4.54 min.

### E) [4-[3-(1-Carbamoylmethyl-1H-[1,2,3]triazol-4-yl)-6-oxo-6H-pyridazin-1-yl]-1-(cis-3-chloro-phenyl)-cyclohexylmethyl]-carbamic acid tert-butyl ester

To (4-{1-[4-(tert-Butoxycarbonylamino-methyl)-4-(cis-3-chloro-phenyl)-cyclohexyl]-6-oxo-1,6-dihydro-pyridazin-3-yl}-[1,2,3]triazol-1-yl)-acetic acid (15 mg, 0.028 mmol) in acetonitrile (1 ml) was added 0-(Benzotriazol-1-yl)-N,N,N′,N′-tetramethyluronium hexafluorophosphate (16 mg, 0.041 mmol) at 0° C. The mixture was stirred at 0° C. for 5 min, then Ammonium carbonate (4 mg, 0.055 mmol) in Triethylamine (0.25 ml) was added to the mixture. The reaction mixture was stirred at room temperature for 16 h. The reaction mixture was treated with saturated aqueous sodium bicarbonate solution and extracted twice with ethyl acetate. The combined organic layers were dried over magnesium sulfate and concentrated in vacuo to give the title compound.

### F) 2-[cis-4-Aminomethyl-4-(3-chloro-phenyl)-cyclohexyl]-6-{1-[2-(3,5-dimethyl-1H-pyrazol-4-yl)-ethyl]-1H-[1,2,3]triazol-4-yl}-2H-pyridazin-3-one hydrochloride

To [4-[3-(1-Carbamoylmethyl-1H-[1,2,3]triazol-4-yl)-6-oxo-6H-pyridazin-1-yl]-1-(cis-3-chloro-phenyl)-cyclohexylmethyl]-carbamic acid tert-butyl ester (15 mg, 0.028 mmol) was added 4N hydrogen chloride solution in dioxane (5 ml). The reaction mixture stirred at room temperature for 2 h, then it was concentrated in vacuo. The residue was purified by prep. HPLC (Macherey-Nagel Nucleosil 100-10 C18, flow 40 ml/min, 21 min method (0-2.0 min 5% ACN, 2.0-17.5 min 5-100% ACN, 17.5-19.5 min 100% ACN, 19.5-21.0 min 100-5% ACN).

Fractions containing the product were lyophilized in vacuo to give the formate salt of the title compound, which was dissolved in methanol and treated with an excess of 2M hydrogen chloride in methanol. Removal of the volatiles gave the title compound as a white solid.

MS (ES⁺): 443 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 2.28 min.

### Example WG5

### 2-[4-Aminomethyl-4-(3-chloro-phenyl)-cyclohexyl]-6-[1-(2-piperidin-1-yl-ethyl)-1H-[1,2,3]triazol-4-yl]-2H-pyridazin-3-one hydrochloride

The title compound was prepared analogously as described in Example WG1, using 2-Piperidino-ethylazide instead of 4-(2-Azidoethyl)-3,5-dimethyl-1H-pyrazole.

MS (ES⁺): 496 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 1.93 min.

### Example WG6

### 2-[cis-4-Aminomethyl-4-(3-chloro-phenyl)-cyclohexyl]-6-(1H-[1,2,3]triazol-4-yl)-2H-pyridazin-3-one hydrochloride

The title compound was prepared analogously as described in Example WG2 step A to C using 2,2-Dimethyl-propionic acid azidomethyl ester instead of 4-(2-Azidoethyl)-3,5-dimethyl-1H-pyrazole to afford 2,2-Dimethyl-propionic acid 4-{1-[4-(tert-butoxycarbonylamino-methyl)-4-(cis-3-chloro-phenyl)-cyclohexyl]-6-oxo-1,6-dihydro-pyridazin-3-yl}-[1,2,3]triazol-1-ylmethyl ester followed by step:

### D) {1-(cis-3-Chloro-phenyl)-4-[6-oxo-3(1H-[1,2,3]triazol-4-yl)-6H-pyridazin-1-yl]-cyclohexylmethyl}-carbamic acid tert-butyl ester

To 2,2-Dimethyl-propionic acid 4-{1-[4-(tert-butoxycarbonylamino-methyl)-4-(cis-3-chloro-phenyl)-cyclohexyl]-6-oxo-1,6-dihydro-pyridazin-3-yl}-[1,2,3]triazol-1-ylmethyl ester (24 mg, 0.040 mmol) in Methanol (1 ml) was added 1M aqueous sodium hydroxide solution (1 ml). The mixture was stirred at room temperature for 30 min. The mixture was filtered and concentrated in vacuo to give the title compound.

### E) 2-[cis-4-Aminomethyl-4-(3-chloro-phenyl)-cyclohexyl]-6-(1H-[1,2,3]triazol-4-yl)-2H-pyridazin-3-one hydrochloride

To {1-(cis-3-Chloro-phenyl)-4-[6-oxo-3-(1H-[1,2,3]triazol-4-yl)-6H-pyridazin-1-yl]-cyclohexylmethyl}-carbamic acid tert-butyl ester (20 mg, 0.041 mmol) was added 4N hydrogen chloride solution in dioxane (5 ml). The reaction mixture stirred at room temperature for 2 h, then it was concentrated in vacuo. The residue was purified by prep. HPLC (Macherey-Nagel Nucleosil 100-10 C18, flow 40 ml/min, 21 min method (0-2.0 min 5% ACN, 2.0-17.5 min 5-100% ACN, 17.5-19.5 min 100% ACN, 19.5-21.0 min 100-5% ACN). Fractions containing the product were lyophilized in vacuo to give the formate salt of the title compound, which was dissolved in methanol and treated with an excess of 2M hydrogen chloride in methanol. Removal of the volatiles gave the title compound as a white solid.

MS (ES⁺): 385 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 2.48 min.

### Example WH1

### 2-[cis-4-Aminomethyl-4-(3-chloro-phenyl)-cyclohexyl]-6-(4-propyl-[1,2,3]triazol-1-yl)-2H-pyridazin-3-one hydrochloride

The title compound was prepared analogously as described in Example WA1, step A to afford [4-(3-Bromo-6-oxo-6H-pyridazin-1-yl)-1-(cis-3-chloro-phenyl)-cyclohexylmethyl]-carbamic acid tert-butyl ester followed by step

### B) [4-(3-Azido-6-oxo-6H-pyridazin-1-yl)-1-(3-chloro-phenyl)-cyclohexylmethyl]-carbamic acid tert-butyl ester

To a solution of [4-(3-Bromo-6-oxo-6H-pyridazin-1-yl)-1-(cis-3-chloro-phenyl)-cyclohexylmethyl]-carbamic acid tert-butyl ester (40 mg, 0.081 mmol) in a mixture of Ethanol (1.4 ml) and water (0.6 ml) was added Sodium Azide (10.5 mg, 0.161 mmol), Copper iodide (1.5 mg, 0.008 mmol), Sodium ascorbate (1 mg, 0.004 mmol) and N—N-Dimethylethylenediamine (1.3 μl, 0.012 mmol). The mixture was stirred at room temperature for 1 h, then treated with microwave at 60° C. for 15 seconds. The mixture was extracted into dichloromethane. The organic phase was dried and concentrated in vacuo to give the title compound.

MS (ES⁺): 403 [M-t-Bu+H]⁺.

### C) {1-(3-Chloro-phenyl)-4-[6-oxo-3-(4-propyl-2,3-dihydro-[1,2,3]triazol-1-yl)-6H-pyridazin-1-yl]-cyclohexylmethyl}-carbamic acid tert-butyl ester

To [4-(3-Azido-6-oxo-6H-pyridazin-1-yl)-1-(3-chloro-phenyl)-cyclohexylmethyl]-carbamic acid tert-butyl ester (37 mg, 0.081 mmol) in a mixture of Dichloromethane (1 ml) and water (1 ml) was added 1-Pentyne (7.9 μl, 0.081 mmol), Copper sulfate (0.6 mg, 0.004 mmol) and Sodium ascorbate (2.4 mg, 0.012 mmol). The mixture was stirred at room temperature for 16 h. The mixture was filtered. The filtrate was concentrated in vacuo to give the title compound.

### D) 2-[cis-4-Aminomethyl-4-(3-chloro-phenyl)-cyclohexyl]-6-(4-propyl-[1,2,3]triazol-1-yl)-2H-pyridazin-3-one hydrochloride

To (1-(3-Chloro-phenyl)-4-[6-oxo-3-(4-propyl-2,3-dihydro-[1,2,3]triazol-1-yl)-6H-pyridazin-1-yl]-cyclohexylmethylycarbamic acid tert-butyl ester (42 mg, 0.08 mmol) was added 4N hydrogen chloride solution in dioxane (5 ml). The reaction mixture stirred at room temperature for 2 h, then it was concentrated in vacuo. The residue was purified by prep. HPLC (Macherey-Nagel Nucleosil 100-10 C18, flow 40 ml/min, 21 min method (0-2.0 min 5% ACN, 2.0-17.5 min 5-100% ACN, 17.5-19.5 min 100% ACN, 19.5-21.0 min 100-5% ACN). Fractions containing the product were lyophilized in vacuo to give the formate salt of the title compound, which was dissolved in methanol and treated with an excess of 2M hydrogen chloride in methanol. Removal of the volatiles gave the title compound as a white solid.

MS (ES⁺): 427 [M+H]⁺.

HPLC (Agilent Eclipse XDB-C18 4.6*50 mm, 1.8 μm, flow 1.5 mL/min, 8 min method (0-6.0 min 20-100% ACN, 6.0-7.5 min 100% ACN, 7.5-8.0 min 100-20% ACN): 3.23 min.

### Example WI1

### 2-[cis-4-Aminomethyl-4-(3-chloro-phenyl)-cyclohexyl]-3-oxo-6-pwidin-3-yl-2,3-dihydro-pyridazine-4-carboxylic acid amide

The title compound was prepared according to Scheme W.

### A) 2-[4-(tert-Butoxycarbonylamino-methyl)-4-(cis-3-chloro-phenyl)-cyclohexyl]-3-oxo-6-pyridin-3-yl-2,3-dihydro-pyridazine-4-carboxylic acid ethyl ester

To a solution of [1-(cis-3-Chloro-phenyl)-4-hydroxy-cyclohexylmethyl]-carbamic acid tert-butyl ester (35 mg, 0.101 mmol), 3-Oxo-6-pyridin-3-yl-2,3-dihydro-pyridazine-4-carboxylic acid ethyl ester (37 mg, 0.151 mmol) and Triphenylphosphine (32 mg, 0.121 mmol) in tetrahydrofurane (40 ml), was added Diethyl azodicarboxylate (26 μl, 0.161 mmol) at room temperature. The reaction mixture was stirred for 3 days at room temperature. The mixture was partitioned between dichloromethane and saturated aqueous sodium bicarbonate solution. The organic layer was dried and concentrated in vacuo. The residue was purified by prep. HPLC (Waters SunFire Prep C18 ODB 5 μm 19×50 mm, flow 20 mL/min, 15 min method (0-2.5 min 5% ACN, 2.5-12.5 min 5-100% ACN, 12.5-15.0 min 100% ACN). Fractions containing the product were partitioned between dichloromethane and saturated aqueous sodium bicarbonate solution. The organic layer was dried and concentrated in vacuo to give the title compound as a yellow solid.

MS (ES⁺): 567 [M+H]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 3.45 min.

### B) [4-(5-Carbamoyl-6-oxo-3-pyridin-3-yl-6H-pyridazin-1-yl)-1-(cis-3-chloro-phenyl)-cyclohexylmethyl]-carbamic acid tert-butyl ester

2-[4-(tert-Butoxycarbonylamino-methyl)-4-(cis-3-chloro-phenyl)-cyclohexyl]-3-oxo-6-pyridin-3-yl-2,3-dihydro-pyridazine-4-carboxylic acid ethyl ester (20 mg, 0.035 mmol) was dissolved in 2M ammonia in Methanol (350 μL, 0.69 mmol). The mixture was stirred at room temperature for 12 h. The mixture was partitioned between dichloromethane and saturated aqueous sodium bicarbonate solution. The organic layer was dried and concentrated in vacuo to give the title compound as a yellow oil.

MS (ES⁺): 538[M+H]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 2070ACN): 3.26 min.

### C) 2-[cis-4-Aminomethyl-4-(3-chloro-phenyl)-cyclohexyl]-3-oxo-6-pyridin-3-yl-2,3-dihydro-pyridazine-4-carboxylic acid amide

Trifluoroacetic acid (56 μl) was added to a solution of [4-(5-Carbamoyl-6-oxo-3-pyridin-3-yl-6H-pyridazin-1-yl)-1-(cis-3-chloro-phenyl)-cyclohexylmethyl]-carbamic acid tert-butyl ester (19 mg, 0.034 mmol) in dichloromethane (2 mL). The reaction mixture was stirred at room temperature for 1 h. The mixture was concentrated in vacuo. The residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were partitioned between dichloromethane and saturated aqueous sodium bicarbonate solution. The organic layer was dried and concentrated in vacuo to give the title compound.

MS (ES⁺): 439 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.81 min.

### Example WI2

### 2-[cis-4-Aminomethyl-4-(3-chloro-phenyl)-cyclohexyl]-3-oxo-6-(1-oxy-pyridin-3-yl)-2,3-dihydro-pyridazine-4-carboxylic acid amide

The title compound was prepared analogously as described in Example WI1, step A to B followed by step

### C) [4-[5-Carbamoyl-6-oxo-3-(1-oxy-pyridin-3-yl)-6H-pyridazin-1-yl]-1-(cis-3-chloro-phenyl)-cyclohexylmethyl]-carbamic acid tert-butyl ester

To a solution of [4-(5-Carbamoyl-6-oxo-3-pyridin-3-yl-6H-pyridazin-1-yl)-1-(cis-3-chloro-phenyl)-cyclohexylmethyl]-carbamic acid tert-butyl ester (50 mg, 0.093 mmol) in dichloromethane (2 ml) was added m-Chloroperbenzoic acid (23 mg, 0.093 mmol). The mixture was stirred at room temperature for 16 h. The mixture was partitioned between dichloromethane and saturated aqueous sodium bicarbonate solution. The organic layer was dried and concentrated in vacuo to give the title compound as a white solid.

MS (ES⁺): 554 [M+H]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 3.42 min.

### D) 2-[cis-4-Aminomethyl-4-(3-chloro-phenyl)-cyclohexyl]-3-oxo-6-(1-oxy-pyridin-3-yl)-2,3-dihydro-pyridazine-4-carboxylic acid amide

Trifluoroacetic acid (35 μl) was added to a solution of [4-[5-Carbamoyl-6-oxo-3-(1-oxy-pyridin-3-yl)-6H-pyridazin-1-yl]-1-(cis-3-chloro-phenyl)-cyclohexylmethyl]-carbamic acid tert-butyl ester (23 mg, 0.045 mmol) in dichloromethane (2 mL). The reaction mixture was stirred at room temperature for 1 h. The mixture was concentrated in vacuo. The residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were partitioned between dichloromethane and saturated aqueous sodium bicarbonate solution. The organic layer was dried and concentrated in vacuo to give the title compound as a yellow solid.

MS (ES⁺): 454 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.87 min.

### Example WJ1

### 4-Amino-2-[cis-4-aminomethyl-4-(3-chloro-phemf1)-cyclohexyl]-6-pyridin-3-yl-2H-pyridazin-3-one

The title compound was prepared analogously as described in Example WI1, step A followed by step

### B) 2-[1-(tert-Butoxycarbonylamino-methyl)-4-(cis-3-chloro-phenyl)-cyclohexyl]-3-oxo-6-pyridin-3-yl-2,3-dihydro-pyridazine-4-carboxylic acid

To 2-[4-(tert-Butoxycarbonylamino-methyl)-4-(cis-3-chloro-phenyl)-cyclohexyl]-3-oxo-6-pyridin-3-yl-2,3-dihydro-pyridazine-4-carboxylic acid ethyl ester (250 mg, 0.442 mmol) in Tetrahydrofurane (2 ml) and water (2 ml) was added Lithium hydroxide hydrate (93 mg, 2.2 mmol). The mixture was stirred at 60° C. for 3 h. The mixture was partitioned between dichloromethane and 1N Hydrochloric acid. The organic layer was dried and concentrated in vacuo to give the title compound as an orange solid.

MS (ES⁺): 540[M+Na]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 3.55 min.

### C) [4-(5-Amino-6-oxo-3-pyridin-3-yl)-6H-pyridazin-1-yl)-1-(cis-3-chloro-phenyl)-cyclohexylmethyl]-carbamic acid tert-butyl ester

To a solution of 2-[4-(tert-Butoxycarbonylamino-methyl)-4-(cis-3-chloro-phenyl)-cyclohexyl]-3-oxo-6-pyridin-3-yl-2,3-dihydro-pyridazine-4-carboxylic acid (200 mg, 0.372 mmol) in Tetrahydrofurane (10 ml) was added at 0° C. N-Methylmorpholine (49 μL, 0.445 mmol) and Isobutylchloroformate (58 μL, 0.445 mmol). The mixture was stirred at 0° C. for 0.5 h, then Sodium azide (36 mg, 0.557 mmol) was added. The mixture was stirred at ° C. for 1 h, then at room temperature for 16 h. The mixture was partitioned between dichloromethane and saturated aqueous sodium bicarbonate solution. The organic layer was dried and concentrated in vacuo to give [4-(5-Azidocarbonyl-6-oxo-3-pyridin-3-yl-6H-pyridazin-1-yl)-1-(cis-3-chloro-phenyl)-cyclohexylmethyl]-carbamic acid tert-butyl ester which was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the azide were kept at room temperature for 16 h to form the amine. Then they were partitioned between dichloromethane and saturated aqueous sodium bicarbonate solution. The organic layer was dried and concentrated in vacuo to give the title compound as a yellow solid.

MS (ES⁺): 510 [M+H]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 3.21 min.

### D) 4-Amino-2-[cis-4-aminomethyl-4-(3-chloro-phenyl)-cyclohexyl]-6-pyridin-3-yl-2H-pyridazin-3-one

Trifluoroacetic acid (28 μl) was added to a solution of [4-(5-Amino-6-oxo-3-pyridin-3-yl-6H-pyridazin-1-yl)-1-(cis-3-chloro-phenyl)-cyclohexylmethyl]-carbamic acid tert-butyl ester (20 mg, 0.036 mmol) in dichloromethane (2 mL). The reaction mixture was stirred at room temperature for 1 h. The mixture was concentrated in vacuo. The residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were partitioned between dichloromethane and saturated aqueous sodium bicarbonate solution. The organic layer was dried and concentrated in vacuo to give the title compound as a yellow solid.

MS (ES⁺): 410 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.79 min.

### Example WJ2

### 4-Amino-2-[4-aminomethyl-4-(3-chloro-phenyl)-cyclohexyl]-6-(1-oxy-mfridin-3-yl)-2H-pyridazin-3-one

The title compound was prepared analogously as described in Example WJ1, step A to C followed by step

### D) [4-[5-Amino-6-oxo-3-(1-oxy-pyridin-3-yl)-6H-pyridazin-1-yl]-1-(cis-3-chloro-phenyl)-cyclohexylmethyl]-carbamic acid tert-butyl ester

To a solution of [4-(5-Amino-6-oxo-3-pyridin-3-yl-6H-pyridazin-1-yl)-1-(cis-3-chloro-phenyl)-cyclohexylmethyl]-carbamic acid tert-butyl ester (50 mg, 0.090 mmol) in dichloromethane (2 ml) was added m-Chloroperbenzoic acid (22 mg, 0.090 mmol). The mixture was stirred at room temperature for 16 h. The mixture was partitioned between dichloromethane and saturated aqueous sodium bicarbonate solution. The organic layer was dried and concentrated in vacuo. The residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were partitioned between dichloromethane and saturated aqueous sodium bicarbonate solution. The organic layer was dried and concentrated in vacuo to give the title compound as a white solid.

MS (ES⁺): 527 [M+H]⁺.

HPLC (Waters Symmetry C18 3.5 μm 2.1×50 mm, 6 min method (0-3 min 20-95% ACN, 3.5-5.5 min 95% ACN, 5.5-5.55 min 95-20% ACN, 5.55-6 min 20% ACN): 3.47 min.

### E) 4-Amino-2-[4-aminomethyl-4-(3-chloro-phenyl)-cyclohexyl]-6-(1-oxy-pyridin-3-yl)-2H-pyridazin-3-one

Trifluoroacetic acid (34 μl) was added to a solution of [4-[5-Amino-6-oxo-3-(1-oxy-pyridin-3-yl)-6H-pyridazin-1-yl]-1-(3-chloro-phenyl)-cyclohexylmethyl]-carbamic acid tert-butyl ester (25 mg, 0.044 mmol) in dichloromethane (2 mL). The reaction mixture was stirred at room temperature for 1 h. The mixture was concentrated in vacuo. The residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were partitioned between dichloromethane and saturated aqueous sodium bicarbonate solution. The organic layer was dried and concentrated in vacuo to give the title compound as a yellow solid.

MS (ES⁺): 427 [M+H]⁺.

HPLC (Waters Symmetry C18 3.5 μm 2.1×50 mm, 6 min method (0-3 min 5-95% ACN, 3.5-5.5 min 95% ACN, 5.5-5.55 min 95-5% ACN, 5.55-6 min 5% ACN): 2.88 min.

### Example WK1

### 2-[cis-4-Aminomethyl-4-(3-chloro-phenyl)-cyclohexyl]-6-morpholin-4-yl-2H-pyridazin-3-one hydrochloride

The title compound was prepared analogously as described in Example WA1, step A to afford [4-(3-Bromo-6-oxo-6H-pyridazin-1-yl)-1-(cis-3-chloro-phenyl)-cyclohexylmethyl]-carbamic acid tert-butyl ester followed by step

### B) [1-(cis-3-Chloro-phenyl)-4-(3-morpholin-4-yl-6-oxo-6H-pyridazin-1-yl)-cyclohexylmethyl]-carbamic acid tert-butyl ester

To a solution of [4-(3-Bromo-6-oxo-6H-pyridazin-1-yl)-1-(cis-3-chloro-phenyl)-cyclohexylmethyl]-carbamic acid tert-butyl ester (30 mg, 0.06 mmol) in toluene (0.9 ml) was added Morpholine (32 μl, 0.362 mmol), (±)-2,2′-Bis(diphenylphosphino)-1,1′-binaphthalene (1 mg, 0.0018 mmol), Tris(dibenzylideneacetone)dipalladium(0) (1 mg, 0.0012 mmol) and Sodium tert.butoxide (8 mg, 0.085 mmol). The mixture was stirred at 120° C. for 20 min. To the mixture was added Morpholine (16 μl, 0.181 mmol), (±)-2,2′-Bis(diphenylphosphino)-1,1′-binaphthalene (0.5 mg, 0.0009 mmol), Tris(dibenzylideneacetone)dipaiiadium(0) (0.5 mg, 0.0006 mmol). The mixture was treated with microwave at 120° C. for 10 minutes. The mixture was filtered over a ChemElut Extraction column (VARIAN), eluting with Ethyl acetate. The filtrate was concentrated in vacuo. The residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were lyophilized in vacuo to give the title compound.

MS (ES⁺): 503 [M+H]⁺.

### C) 2-[cis-4-Aminomethyl-4-(3-chloro-phenyl)-cyclohexyl]-6-morpholin-4-yl-2H-pyridazin-3-one hydrochloride

To [1-(cis-3-Chloro-phenyl)-4-(3-morpholin-4-yl-6-oxo-6H-pyridazin-1-yl)-cyclohexylmethyl]-carbamic acid tert-butyl ester (33 mg, 0.076 mmol) was added 4N hydrogen chloride solution in dioxane (5 ml). The reaction mixture stirred at room temperature for 2 h, then it was concentrated in vacuo. The residue was treated with diethyl ether in ultrasonic bath. The etheric phase was removed with a pipette. The residue was lyophilized in vacuo to give the title compound as a white solid.

MS (ES⁺): 403 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.82 min.

### Example WK2

### 6-(4-Acetyl-piperazin-1-yl)-2-[cis-4-aminomethyl-4-(3-chloro-phenyl)-cyclohexyl]-2H-pyridazin-3-one hydrochloride

The title compound was prepared analogously as described in Example WK1, using 1-Acetylpiperazine instead of Morpholine.

MS (ES⁺): 444[M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.71 min.

### Example WK3

### 4-{1-[4-Aminomethyl-4-(3-chloro-phenyl)-cyclohexyl]-6-oxo-1,6-dihydro-pyridazin-3-yl}-morpholine-2-carboxylic acid methylamide hydrochloride

The title compound was prepared analogously as described in Example WK1, using Morpholine-2-carboxylic acid methylamide instead of Morpholine.

MS (ES⁺): 460[M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.79 min.

### Example WK4

### 2-[4-Aminomethyl-4-(3-chloro-phenyl)-cyclohexyl]-6-piperidin-1-yl-2H-pyridazin-3-one hydrochloride

The title compound was prepared analogously as described in Example WK1, using Piperidine instead of Morpholine.

MS (ES⁺): 401[M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 4.35 min.

### Example WL1

### 2-[cis-4-Aminomethyl-4-(3-chloro-phenyl)-cyclohexyr]-6-(3-oxo-piperazin-1-yl)-2H-pyridazin-3-one hydrochloride

The title compound was prepared analogously as described in Example WA1, step A to afford [4-(3-Bromo-6-oxo-6H-pyridazin-1-yl)-1-(cis-3-chloro-phenyl)-cyclohexylmethyl]-carbamic acid tert-butyl ester followed by step

### B) {1-(cis-3-Chloro-phenyl)-4-[6-oxo-3-(3-oxo-piperazin-1-yl)-6H-pyridazin-1-yl]-cyclohexylmethyl}-carbamic acid tert-butyl ester

To a solution of [4-(3-Bromo-6-oxo-6H-pyridazin-1-yl)-1-(cis-3-chloro-phenyl)-cyclohexylmethyl]-carbamic acid tert-butyl ester (30 mg, 0.06 mmol) in Dimethylsulfoxide (0.72 ml) was added piperazin-2-one (18 mg, 0.181 mmol), Copper(1)iodide (2.3 mg, 0.012 mmol), L-Proline (2.8 mg, 0.024 mmol) and Potassium carbonate (17 mg, 0.121 mmol). The mixture was stirred at 90° C. for 16 h. The mixture was directly purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were lyophilized in vacuo to give the title compound.

MS (ES⁺): 516 [M+H]⁺.

### C) 2-[cis-4-Aminomethyl-4-(3-chloro-phenyl)-cyclohexyl]-6-(3-oxo-piperazin-1-yl)-2H-pyridazin-3-one

To {1-(cis-3-Chloro-phenyl)-4-[6-oxo-3-(3-oxo-piperazin-1-yl)-6H-pyridazin-1-yl]-cyclohexylmethyl}-carbamic acid tert-butyl ester (9 mg, 0.017 mmol) was added 4N hydrogen chloride solution in dioxane (4 ml). The reaction mixture stirred at room temperature for 2 h, then it was concentrated in vacuo. The residue was treated with diethyl ether in ultrasonic bath. The etheric phase was removed with a pipette. The residue was lyophilized in vacuo to give the title compound as a white solid.

MS (ES⁺): 416 [M+H]⁺.

HPLC (Nucleosil C-18HD 4×70 mm 3 μm, 8 min method (0-6 min 5-100% ACN, 6.0-7.5 min 100% ACN, 7.5-8.0 min 100-5% ACN): 3.56 min.

### Example. Y1

### C-[cis-4-(5,6,7,8-Tetrahydro-naphthalen-1-yl)-1-m-tolyl-cyclohexyl]-methylamine hydrochloride

The title compound was prepared according to Scheme Y.

### A) 4-Cyano-4-m-tolyl-heptanedioic acid dimethyl ester

To a solution of Triton B (25.5 mL, 61 mmol of a 40% solution in methanol) in t-butanol (30 mL) was added a solution of 3-Methylbenzylcyanide (25 ml, 185 mmol) and Methyl acrylate (47.2 mL, 519 mmo t-butanol (70 ml). When the addition was complete, the reaction mixture was stirred at 80° C. for 16 h. After cooling, the reaction mixture was treated with 4N Hydrochloric acid to pH2, then concentrated vacuo. The residue aqueous phase was extracted 2× with ethyl acetate. The combined organic pha were dried over Magnesium sulfate and concentrated in vacuo. The residue was recrystallised from diethyl ether:pentane 1:1 to give the title compound as a white solid.

MS (ES⁺): 321 [M+H2O]⁺. HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 6.29 min.

### B) 5-Cyano-2-oxo-5-m-tolyl-cyclohexanecarboxylic acid methyl ester

To a solution of 4-Cyano-4-m-tolyl-heptanedioic acid dimethyl ester (10.4 g, 34.3 mmol) in tetrahydrofurane (100 ml) was added Potassium tert-butoxide (9.4 g, 78.7 mmol). The resulting mixture was stirred at 70° C. for 2 h. The reaction mixture was cooled (0° C.) and treated with a solution of acetic acid (12 mL) in water (60 mL). The mixture was extracted with diethyl ether and the organic phase was washed with 2N aqueous sodium bicarbonate solution and water, then dried over Magnesium sulfate and concentrated in vacuo to give the title compound as a white solid.

MS (ES⁺): 289 [M+H2O]⁺.

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 6.66 min.

### C) 4-Oxo-1-m-tolyl-cyclohexanecarbonitrile

A mixture of 5-Cyano-2-oxo-5-m-tolyl-cyclohexanecarboxylic acid methyl ester (7.4 g, 27.3 mmol) in 10% aqueous sulphuric acid (40 mL) and acetic acid (80 mL) was stirred for 16 h at 110° C. After cooling to room temperature, the reaction mixture was diluted with water and extracted into ethyl acetate. The organic phase was washed with 2N aqueous sodium bicarbonate solution and water, then dried over Magnesium sulfate and concentrated in vacuo to give the title compound as an orange oil.

MS (ES⁺): 426 [2×M+H]⁺.

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 6.02 min.

### D) Trifluoro-methanesulfonic acid 4-cyano-4-m-tolyl-cyclohex-1-enol ester

To a solution of Lithium diisopropylamide solution (2.8 ml, 5.6 mmol of a 2.0 M solution in tetrahydrofuran/heptane/ethylbenzene) in tetrahydrofurane (10 ml) was added dropwise a solution of 4-Oxo-1-m-tolyl-cyclohexanecarbonitrile (1.0 g, 4.64 mmol) in tetrahydrofurane (5 ml) at −78° C. The resulting mixture was stirred at −78° C. for 30 minutes, then a solution of N-Phenyl-bis(trifluoromethansulfonimide) (1.99 g, 5.57 mmol) in tetrahydrofurane (5 ml) was added. The reaction mixture was stirred at 0° C. for 5 h. The mixture was concentrated in vacuo. The residue was partitioned between dichloromethane and 1N Hydrochloric acid. The organic phase was dried over Magnesium sulfate and concentrated in vacuo to give the title compound.

MS (ES⁺): 289 [M+H2O]⁺.

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 6.66 min.

### E) 4-Naphthalen-1-yl-1-m-tolyl-cyclohex-3-enecarbonitrile

To a solution of Trifluoro-methanesulfonic acid 4-cyano-4-m-tolyl-cyclohex-1-enyl ester (1.25 g, 2.32 mmol) in 1,2-Dimethoxyethane (10 ml) was added 1-Naphthaleneboronic acid (558 mg, 3.24 mmol), Lithium chloride (295 mg, 6.96 mmol), Tetrakis(triphenylphosphine)palladium(0) (135 mg, 0.116 mmol) and 2N aqueous sodium carbonate solution (3 ml). The reaction mixture was stirred for 3 h at 90° C. After cooling, the mixture was partitioned between dichloromethane and saturated aqueous sodium bicarbonate solution. The organic layer was dried over Magnesium sulfate and concentrated in vacuo. The residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were concentrated in vacuo to give the title compound.

MS (ES⁺): 341 [M+H2O]⁺.

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 7.97 min.

### F) C-[cis-4-(5,6,7,8-Tetrahydro-naphthalen-1-yl)-1-m-tolyl-cyclohexyl]-methylamine hydrochloride and C-[trans-4-(5,6,7,8-Tetrahydro-naphthalen-1-yl)-1-m-tolyl-cyclohexyl]-methylamine hydrochloride

To a solution of 4-Naphthalen-1-yl-1-m-tolyl-cyclohex-3-enecarbonitrile (260 mg, 0.804 mmol) in Ethanol (25 ml) and conc. Hydrochloric acid (5 ml, 37%) was added Platinum(IV)oxide hydrate (18.3 mg, 0.081 mmol). The reaction mixture was stirred at room temperature for 3 h under hydrogen atmosphere. The mixture was filtered, then the filtrate was concentrated in vacuo. The residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were lyophilized in vacuo to give the formate salts of the individual title compounds, which were dissolved in methanol and treated with an excess of 2M hydrogen chloride in methanol. Removal of the volatiles gave the individual title compounds as white solids.

MS (ES⁺): 334 [M+H]⁺ (cis) and MS (ES⁺): 334 [M+H]⁺ (trans)

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 6.55 min (cis) and 6.44 min (trans).

### Example Y2

### C-[cis-4-(4,5,6,7,8-Tetrahydro-naphthalen-2-yl)-1-m-tolyl-cyclohexyl]-methylamine hydrochloride

The title compound was prepared analogously as described in Example Y1, using 2-Naphthaleneboronic acid instead of 1-Naphthaleneboronic acid.

MS (ES⁺): 334 [M+H]⁺

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 6.66 min

### Example Y3

### C-(cis-4-Naphthalen-1-yl-1-m-tolyl-cyclohexyl)-methylamine hydrochloride

The title compound was prepared analogously as described in Example Y1, step A to E followed by step

### F) C-(4-Naphthalen-1-yl-1-m-tolyl-cyclohex-3-enyl)-methylamine

To a solution of 4-Naphthalen-1-yl-1-m-tolyl-cyclohex-3-enecarbonitrile (280 mg, 0.866 mmol) in Diethylether (10 ml), was added Lithiumaluminium hydride (85 mg, 2.16 mmol). The resulting mixture was stirred at room temperature for 2 h. The mixture was treated with aqueous Potassium sodium tartrate solution and extracted 2× into ethyl acetate. The combined organic phases were dried over Magnesium sulfate and concentrated in vacuo to give the title compound.

MS (ES⁺): 328 [M+H]⁺

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 8.32 min.

### G) C-(cis-4-Naphthalen-1-yl-1-m-tolyl-cyclohexyl)-methylamine hydrochloride and C-(trans-4-Naphthalen-1-yl-1-m-tolyl-cyclohexyl)-methylamine hydrochloride

To a solution of C-(4-Naphthalen-1-yl-1-m-tolyl-cyclohex-3-enyl)-methylamine (250 mg, 0.687 mmol) in Ethanol (5 ml) was added 10% Palladium on charcoal (73 mg, 0.069 mmol). The reaction mixture was stirred at room temperature for 16 h under hydrogen atmosphere. The mixture was filtered, then the filtrate was concentrated in vacuo. The residue was purified by prep. HPLC (Waters SunFire Prep C18 ODB 5 μm 19×50 mm, flow 20 ml/min, 15 min method (0-2.5 min 5% ACN, 2.5-12.5 min 5-100% ACN, 12.5-15.0 min 100% ACN). Fractions containing the product were lyophilized in vacuo to give the formate salts of the individual title compounds, which were dissolved in methanol and treated with an excess of 2M hydrogen chloride in methanol. Removal of the volatiles gave the individual title compounds as white solids.

MS (ES⁺): 330 [M+H]⁺ (cis) and MS (ES⁺): 330 [M+H]⁺ (trans)

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 5.30 min (cis) and 5.18 min (trans).

### Example Y4

### C-(cis-4-Naphthalen-2-yl-1-m-tolyl-cyclohexyl)-methylamine hydrochloride

The title compound was prepared analogously as described in Example Y3, using 2-Naphthaleneboronic acid instead of 1-Naphthaleneboronic acid.

MS (ES⁺): 330 [M+H]⁺

HPLC (Macherey-Nagel LiChrospher 100-5 RP18 Flow 1.5 mL/min, 12 min method (0-1.5 min 10% ACN, 1.5-6.5 min 10-100% ACN, 6.5-9.0 min 100% ACN, 9.0-12.0 min 100-10% ACN): 5.38 min

### Example AA

### Activity Assay

Various Example compounds were tested for their inhibitory activity to human DPP-IV.

### Materials

Human DPP-IV consisting of amino acids 39 to 766 followed by a C-terminal Streptavidin-tag was expressed using the baculovirus system and purified to >80% purity. The enzyme was stored in 25 mM Tris buffer, pH 9.0, containing 300 mM NaCl at −80° C.

The fluorogenic substrates H-Gly-Pro-AMC was purchased from Bachem AG (Bubendorf, Switzerland). The substrate was kept as a 5 mM stock solution in DMSO at −20° C. All other chemicals were purchased from Sigma (Buchs, Switzerland).

The assay buffer for the DPP-IV reaction was 25 mM Tris/HCl, pH 7.5, containing 140 mM NaCl, 10 mM KCl and 0.05% (w/v) CHAPS.

### Compound and Liquid Handling

The test compounds were dissolved in 90% DMSO/10% H2O (v/v). Serial dilutions of the compounds from 3 mM to 0.03 μM in 90% DMSO/10% H2O (v/v) followed by a 1:33.3 dilution in assay buffer was done in 96-well polypropylene plates using a CyBio Dilus 8-channel pipettor (CyBio AG, Jena, Germany) with tip change after each pipetting step. The compound solutions as well as the substrate and the enzyme solutions were transferred to the assay plates (384-well black Cliniplate; cat. no. 95040020 Labsystems Oy, Finland) by means of a CyBi-Well 96-channel pipettor (CyBio AG, Jena, Germany).

### Kinetic Measurements

Enzyme kinetics were measured by mixing 10 μl of a 3-fold concentrated substrate solution in assay buffer (final substrate concentration was 10 μM) with 10 μl of the corresponding compound solution. The reactions were initiated by addition of 10 μl of a 3-fold concentrated solution of the enzyme in assay buffer. Final enzyme (active site) concentrations in the assay was 10 μM for DPP-IV. Fluorescence product (AMC) formation was monitored for 1 hour at room temperature at 35 second intervals by measuring the fluorescence emission at 500 nm using an exitation wavelength of 350 nm in a TECAN Ultra fluorescence reader (TECAN, Maennedorf, Switzerland). The fluorescence in each well was excited by one flash per measurement. The Origin software package (Origin 7.5 Mircocal, Northampton, Mass., USA) was used to generate all graphs and to perform the IC50 calculations.

### Results

The inhibitory activities (IC₅₀ values) of the compounds to human DPP-IV were found to be 50 μM or less and in many cases 10 μM or less. The activity data of selected compounds are shown in the table below.

| | IC₅₀ |
|---|---|
| Compound | (μM) |
| 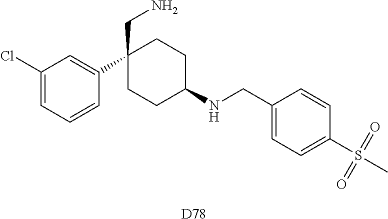 [SMILES: CS(=O)(=O)c1ccc(CN[C@H]2CC[C@](CN)(c3cccc(Cl)c3)CC2)cc1 ; "D78"] | 0.4 |
| 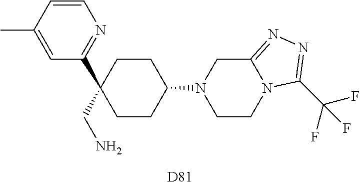 [SMILES: Cc1ccnc([C@]2(CN)CC[C@@H](N3CCn4c(nnc4C(F)(F)F)C3)CC2)c1 ; "D81"] | 0.25 |
| 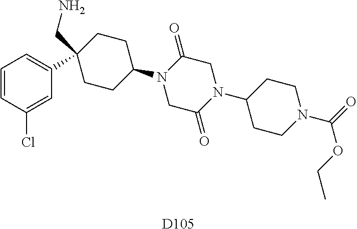 [SMILES: CCOC(=O)N1CCC(N2CC(=O)N([C@H]3CC[C@](CN)(c4cccc(Cl)c4)CC3)CC2=O)CC1 ; "D105"] | 0.1 |
| 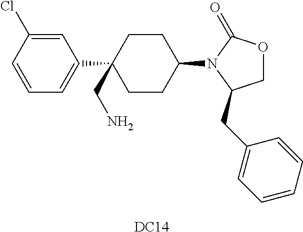 [SMILES: NC[C@]1(c2cccc(Cl)c2)CC[C@H](N2C(=O)OC[C@H]2Cc2ccccc2)CC1 ; "DC14"] | 0.2 |
| 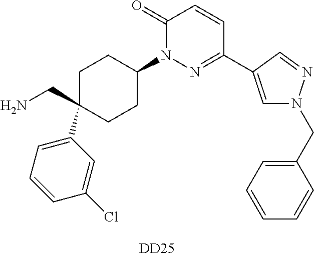 | 0.5 |
| 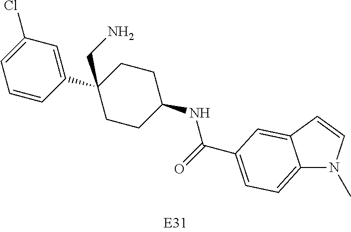 [SMILES: Cn1ccc2cc(C(=O)N[C@H]3CC[C@](CN)(c4cccc(Cl)c4)CC3)ccc21] | 0.05 |
| 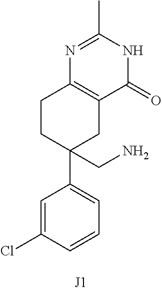 [SMILES: Cc1nc2c(c(=O)[nH]1)CC(CN)(c1cccc(Cl)c1)CC2] | 11.65 |
| 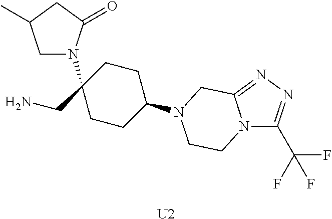 [SMILES: CC1CC(=O)N([C@]2(CN)CC[C@@H](N3CCn4c(nnc4C(F)(F)F)C3)CC2)C1 ; "U2"] | 0.85 |
| 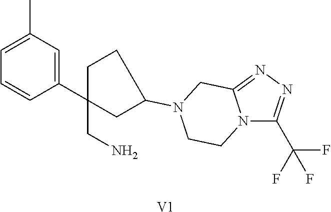 [SMILES: Cc1cccc(C2(CN)CCC(N3CCn4c(nnc4C(F)(F)F)C3)C2)c1 ; "V1"] | 5.05 |
| 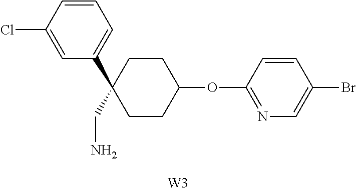 | 0.2 |
| 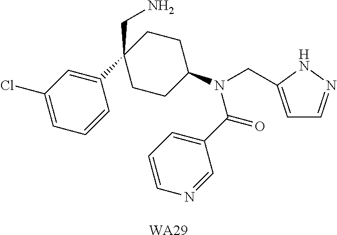 | 0.45 |
| 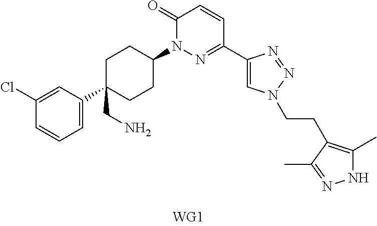 [SMILES: Cc1n[nH]c(C)c1CCn1cc(-c2ccc(=O)n([C@H]3CC[C@](CN)(c4cccc(Cl)c4)CC3)n2)nn1] | 0.85 |

1. A compound of Formula (I): 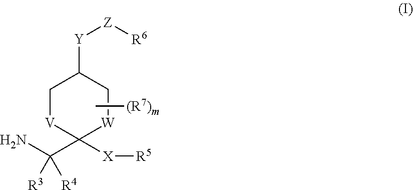
wherein
one of V and W is selected from a bond, —(CH₂)ₙ—, —O—, —NH— and —N(R⁸)—; and the other is selected from —(CH₂)ₙ— and —O—;
X is a bond or a linker having 1 to 5 in-chain atoms and comprising one or more linkages selected from —O—, —C(O)—, —S(O)ₗ—, —N(R⁸)— and hydrocarbylene optionally substituted with 1, 2, 3, 4 or 5 R¹⁰; with the proviso that, when at least one of V and W is —O—, —NH— or —N(R⁸)—, X is a bond;
Y is a bond; or Y and an R⁷ moiety taken together with the atom(s) to which they are attached form a carbocycle or a heterocycle, either of which is optionally substituted with 1, 2, 3, 4 or 5 R¹⁰;
Z is a bond or a linker having 1 to 12 in-chain atoms and comprising one or more linkages selected from —O—, —C(O)—, —S(O)ₗ, —N(R⁸)—, hydrocarbylene optionally substituted with 1, 2, 3, 4 or 5 R¹⁰, and heterocyclylene optionally substituted with 1, 2, 3, 4 or 5 R¹⁰;
R³ and R⁴ are each independently hydrogen or R¹⁰; or R³ and R⁴ taken together with the carbon atom to which they are attached form carbocyclyl or heterocyclyl, either of which is optionally substituted with 1, 2, 3, 4 or 5 R¹⁰;
R⁵ is selected from hydrogen, except when X is a bond; hydrocarbyl optionally substituted with 1, 2, 3, 4 or 5 R¹⁰;
R⁶ is selected from hydrogen, except when Y and Z are each a bond; hydrocarbyl optionally substituted with 1, 2, 3, 4 or 5 R¹⁰; and —(CH₂)ₖ-heterocyclyl optionally substituted with 1, 2, 3, 4 or 5 R¹⁰;
R⁷ is independently selected from R¹⁰;
or two R⁷ moieties taken together may form a bridge between the atoms to which they are attached, wherein the bridge is a hydrocarbylene or —(CH₂)ᵢ—O—(CH₂)ⱼ— bridge, wherein i and j are each independently 0, 1 or 2;
R⁸ is selected from R⁹, —OR⁹, —C(O)R⁹, —C(O)OR⁹ and —S(O)ₗR⁹;
R⁹ is selected from hydrogen; hydrocarbyl optionally substituted with 1, 2, 3, 4 or 5 R¹⁰; and —(CH₂)ₖ-heterocyclyl optionally substituted with 1, 2, 3, 4 or 5 R¹⁰;
each R¹⁰ is independently selected from halogen, trifluoromethyl, cyano, nitro, oxo, ═NR¹¹, —OR¹¹, —C(O)R¹¹, —C(O)OR¹¹, —OC(O)R¹¹, —S(O)ₗR¹¹, —N(R¹¹)R¹², —C(O)N(R¹¹)R¹², —S(O)ₗN(R¹¹)R¹² and R¹³;
R¹¹ and R¹² are each independently hydrogen or R¹³;
R¹³ is selected from hydrocarbyl and —(CH₂)ₖ-heterocyclyl, either of which is optionally substituted with 1, 2, 3, 4 or 5 substituents independently selected from halogen, cyano, amino, hydroxy, C₁₋₆ alkyl and C₁₋₆ alkoxy;
k is 0, 1, 2, 3, 4, 5 or 6;
l is 0, 1 or 2;
m is 0, 1, 2, 3, 4, 5 or 6; and
n is 1 or 2;
or a pharmaceutically acceptable salt or prodrug thereof.

2. The compound according to claim 1, wherein the compound is of the Formula (VII): 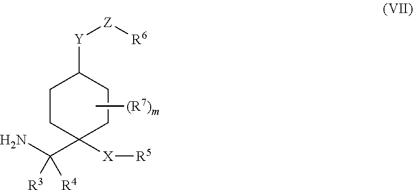
or a pharmaceutically acceptable salt or prodrug thereof.

3. The compound according to any preceding claim, X is a bond, —CH₂— or —CH₂O—; and R⁵ is phenyl optionally substituted with 1, 2, 3, 4 or 5 R¹⁰.

4. The compound according to claim 3, wherein the compound is of the formula (XVIII): 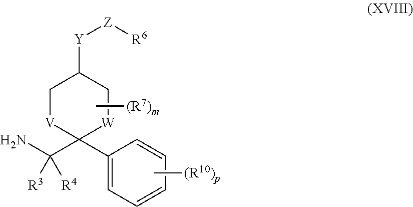
wherein p is 0, 1, 2, 3, 4 or 5;
or a pharmaceutically acceptable salt or prodrug thereof.

5. The compound according to claim 4, wherein, when p is 1, 2, 3, 4 or 5, at least one R¹⁰ is halogen or C₁₋₆ alkyl.

6. The compound according to claim 5, wherein, when p is 1, 2, 3, 4 or 5, at least one R¹⁰ is halogen.

7. The compound according to claim 6, wherein, when p is 1, 2, 3, 4 or 5, at least one R¹⁰ is fluorine or chlorine.

8. The compound according to any preceding claim, wherein R³ and R⁴ are each hydrogen.

9. The compound according to claim 8, wherein the compound is of the formula (XXXVI): 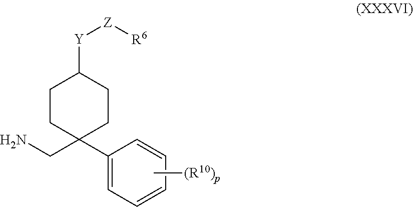
or a pharmaceutically acceptable salt or prodrug thereof.

10. The compound according to claim 9, wherein, when p is 1, 2, 3, 4 or 5, at least one R¹⁰ is halogen or alkyl.

11. The compound according to claim 10, wherein, when p is 1, 2, 3, 4 or 5, at least one R¹⁰ is halogen.

12. The compound according to claim 11, wherein, when p is 1, 2, 3, 4 or 5, at least one R¹⁰ is fluorine or chlorine.

13. The compound according to any preceding claim, m is 0 or 1.

14. The compound according to any preceding claim, wherein Y is a bond.

15. The compound according to any of claims 1 to 13, wherein Y and an R⁷ moiety taken together with the atom(s) to which they are attached form a carbocycle or a heterocycle, either of which is optionally substituted with 1, 2, 3, 4 or 5 R¹⁰.

16-41. (canceled)

42. A method of treating a disease or condition in a patient selected from non-insulin-dependent diabetes mellitus, arthritis, obesity, allograft transplantation, calcitonin-osteoporosis; heart failure, impaired glucose metabolism or impaired glucose tolerance, neurodegenerative diseases, renal diseases, neurodegenerative or cognitive disorders, hyperglycemia, insulin resistance, dyslipidemia, hypertriglyceridemia, hypercholesterolemia, vascular restenosis, irritable bowel syndrome, inflammatory bowel disease, pancreatitis, retinopathy, nephropathy, neuropathy, syndrome X, ovarian hyperandrogenism (polycystic ovarian syndrome), type 2 diabetes, growth hormone deficiency, neutropenia, neuronal disorders, tumor metastasis, benign prostatic hypertrophy, gingivitis, hypertension and osteoporosis; or for producing a sedative or anxiolytic effect, attenuating post-surgical catabolic changes or hormonal responses to stress, reducing mortality and morbidity after myocardial infarction, comprising:
administering a therapeutically effective amount of the compound according to claim 1.

43-53. (canceled)

54. A pharmaceutical formulation, comprising:
the compound of claim 1, and
a pharmaceutically acceptable excipient or carrier.

55. (canceled)

56. A formulation according to claim 54, which further comprises a therapeutic agent selected from anti-diabetic agents, hypolipidemic agents, anti-obesity or appetite-regulating agents, anti-hypertensive agents, HDL-increasing agents, cholesterol absorption modulators, Apo-A1 analogues and mimetics, thrombin inhibitors, aldosterone inhibitors, inhibitors of platelet aggregation, estrogen, testosterone, selective estrogen receptor modulators, selective androgen receptor modulators, chemotherapeutic agents, and 5-HT₃ or 5-HT₄ receptor modulators; or pharmaceutically acceptable salts or prodrugs thereof.

57-62. (canceled)
